(19) United States
(12) Patent Application Publication
(10) Pub. No.: US 2009/0143279 A1
(43) Pub. Date: Jun. 4, 2009

(54) Methods and compositions for treating metabolic disorders

(76) Inventors: Vamsi Krishna Mootha, Cambridge, MA (US); Bridget Wagner, Medford, MA (US); Toshimori Kitami, Cambridge, MA (US)

Correspondence Address: ROPES & GRAY LLP, PATENT DOCKETING 39/41, ONE INTERNATIONAL PLACE, BOSTON, MA, 02110-2624, US

(21) Appl. No.: 12/157,824

(22) Filed: Jun. 13, 2008

Related U.S. Application Data
(60) Provisional application No. 60/934,678, filed on Jun. 15, 2007.
(60) Provisional application No. 61/066,884, filed on Feb. 22, 2008.

(51) Int. Cl. A61K 38/28; A61K 31/4184; A61K 31/407; A61K 31/352; A61K 31/12; A61K 31/337; A61K 31/357; C12Q 1/68; C07H 21/00; C12Q 1/34; A61K 31/4748; A61K 31/475; A61P 3/04; A61P 3/10; A61P 25/00; A61P 9/00

(52) U.S. Cl. 514/4; 514/395; 514/411; 514/456; 514/685; 514/449; 514/463; 514/281; 514/285; 435/6; 435/18; 536/24.33

(57) ABSTRACT

The present invention provides methods of treating of disorders characterized by defective mitochondrial activity. In particular compounds of the present invention can be used in the treatment metabolic diseases and neurodegenerative diseases. The methods are also useful to increase oxidative phosphorylation or to decrease reactive oxygen species (ROS) production in a subject in need thereof.

### RELATED APPLICATIONS

This application claims the benefit of U.S. Provisional Patent Application Nos. 60/934,678 filed Jun. 15, 2007 and 61/066,884 filed Feb. 22, 2008, which applications are hereby incorporated by reference in their entirety.

### FIELD OF THE INVENTION

The present invention provides methods and compositions for treating and preventing metabolic disorders and neurodegenerative disorders, including glucose intolerance and diabetes.

### INTRODUCTION

Mitochondria are cellular structures that represent the center-state for energy homeostasis, programmed cell death, and intermediary metabolism. Inherited or acquired defects in mitochondria can give rise to disease pathogenesis. For example, mutations in genes encoding mitochondrial proteins collectively constitute the largest class of inborn errors of metabolism. We have previously shown that dysfunction in this organelle can give rise to degenerative diseases, such as type 2 diabetes. Dysfunction in this organelle can accompany neurodegeneration and the aging process itself.

A variety of different pathologic phenotypes can emerge out of a particular point mutation in mitochondrial DNA. Clinical symptoms in congenital mitochondrial diseases often manifest in postmitotic tissues with high energy demands like brain, muscle, optic nerve, and myocardium, but other tissues including endocrine glands, liver, gastrointestinal tract, kidney, and hematopoietic tissue are also involved, again depending in part on the segregation of mitochondria during development, and on the dynamics of mitochondrial turnover over time.

In addition to congenital disorders involving inherited defective mitochondria, acquired mitochondrial dysfunction contributes to diseases, particularly neurodegenerative disorders associated with aging like Parkinson's, Alzheimer's, Huntington's Diseases. The incidence of somatic mutations in mitochondrial DNA rises exponentially with age; diminished respiratory chain activity is found universally in aging people. Mitochondrial dysfunction is also implicated in excitotoxic neuronal injury, such as that associated with seizures or ischemia.

Treatment of diseases involving mitochondrial dysfunction has involved administration of vitamins and cofactors used by particular elements of the mitochondrial respiratory chain. Coenzyme Q (ubiquinone), nicotinamide, riboflavin, carnitine, biotin, and lipoic acid are used in patients with mitochondrial disease, with occasional benefit, especially in disorders directly stemming from primary deficiencies of one of these cofactors. However, while useful in isolated cases, no such metabolic cofactors or vitamins have been shown to have general utility in clinical practice in treating mitochondrial diseases. Similarly, dichloracetic acid (DCA) has been used to treat mitochondrial cytopathies such as MELAS; DCA inhibits lactate formation and is primarily useful in cases of mitochondrial diseases where excessive lactate accumulation itself is contributing to symptoms. However, DCA does not address symptoms related to mitochondrial insufficiency per se and can be toxic to some patients, depending on the underlying molecular defects.

A need remains for compositions and methods for treating disorders or pathophysiology associated with mitochondrial dysfunction or mitochondrial respiratory chain dysfunction in a mammal, including humans. The invention provides such methods and compositions.

### BRIEF DESCRIPTION OF THE DRAWINGS

FIGS. 1A-B show C2C12 myotubes in a 384-well format. FIG. 1A: myotubes were differentiated in 384-well format with 4 day starvation (2% horse serum). Tube-like structures are shown using anti-myosin heavy-chain and multinucleus with Hoechst stain. FIG. 1B: Distribution of nuclei for myotubules in a single 384-well. Automated cell counting shows consistent seeding density of 5313+/−384 nuclei per well.

FIG. 2 illustrates the schematic overview of gene expression-based high-throughput screening (GE-HTS) technology. mRNA from cell lysates is captured by 384-well plates coated with oligo-dT, and reverse transcribed to synthesize cDNA. Each target gene is assayed by primer pairs, with gene-specific target sequences that bind adjacently on the corresponding cDNA. Primer pairs are ligated only if they are bound to cDNA, such that the number of ligated products is equal to the copy number of the corresponding cDNA. The ligated products are PCR-amplified using universal primer pairs, and captured with an anti-tag sequence selected for each gene. Each anti-tag sequence is attached to colored beads, and the PCR products are stained with streptavidin-phycoerythrin (SAPE). Dual-color flow cytometry detects bead color in order to identify each gene, and quantifies the amount of SAPE fluorescence to quantify transcript levels.

FIG. 3 shows a schematic used for complementary profiles of viability, mitochondrial physiology and gene expression across 2,490 chemical perturbations. The calcein assay (1) measures cell viability and filters out overtly toxic compounds, such as staurosporine. The MTT assay (2) measures cellular dehydrogenase activity, which is inhibited by the complex I inhibitor rotenone. The JC-1 assay (3) measures the mitochondrial membrane potential (ΔΨm) and drops acutely after the addition of the mitochondrial uncoupler carbonyl cyanide m-chlorophenylhydrazone (CCCP). A luciferase-based assay measures ATP (4), which is reduced by staurosporine. CM-H2DCFDA is a fluorescent probe of cellular ROS (5), which can be stimulated by the addition of H2O2. The expression of both nuOXPHOS and mtOXPHOS transcripts is measured by a multiplex PCR technique, GE-HTS (6). Each column of the heat map represents one sample replicate; expression levels for each gene are row-normalized. Treatment with PGC-1α, an inducer of OXPHOS gene expression, is used as a positive control. All assays were performed in biological duplicate in 384-well format after 48 h of treatment in differentiated murine C2C12 myotubes. Data from 2,490 distinct compounds are incorporated into the screening compendium.

FIG. 4 shows two complementary strategies to identify small molecules that boost OXPHOS gene expression and decrease ROS levels. (a) Mining the compendium for sets of structurally related compounds that achieve the desired activity. All compounds were organized into 624 clusters based on the chemical descriptors molecular weight, log P, number of hydrogen bond donors and acceptors, and number of rotatable bonds. The Mann-Whitney rank-sum statistic for each cluster and each assay was then calculated. The significance of each cluster in each assay is shown, with points above zero indicating positive composite scores and points below zero showing negative composite scores. A nominal P=0.01 is delimited by the dashed lines. The black data points spotlight a single cluster that is significant for the desired activity, with the shared chemical scaffold shown. (b) Mining the compendium for individual compounds that achieve the desired activity. The distributions of ROS scores are shown for all compounds (gray) and for compounds associated with the highest OXPHOS gene expression (black). The latter follow a bimodal distribution, and the smaller mode (bracketed) contains six compounds that elevate OXPHOS expression and decrease ROS levels, with chemical structures shown.

FIG. 5 shows how cell-based assays provide complementary information. a, Pairwise correlation coefficients between assays using composite Z-scores for all 2490 compounds tested. b, Pairwise correlation coefficients between all assays using composite Z-scores after filtering for low-signal outliers (p<0.05) in the viability assay.

FIG. 6 shows the secondary analyses of the effects of microtubule inhibitors on OXPHOS gene expression and physiology. (a) Compounds indicated in FIG. 4 were retested at 20 nM, 200 nM, 2 μM and 20 μM. Gene expression levels are represented as a row-normalized heat map, with negative controls (DMSO treatment) and positive controls (PGC-1α treatment) shown. Dose-response curves for ROS levels and viability are also provided, where the y-axis is the composite Z-score. Shaded area indicates the noise envelope (P<0.05). Data shown are the results of four biological replicates per concentration. (b) Analysis of mtDNA/nuDNA copy number ratio after treatment with four of the compounds (deo, deoxysappanone B; meb, mebendazole; noc, nocodazole; pac, paclitaxel), using three biological replicates, normalized to DMSO treatment alone. (c) Quantitative PCR measurement of Ppargc1a gene expression, in response to either DMSO alone (Con), 5 μM deoxysappanone B (deo) or 1 μM mebendazole (meb). (d) Quantitative PCR measurement of the nuclear OXPHOS gene Atp5a1. Cells were either treated with compound alone (black bars) or in combination with 5 mM of the ERRa inverse agonist XCT790 (gray bars). (e) Quantitative PCR measurement of Sod2, which encodes the ROS scavenger MnSOD, as in (d). Means and s.d. of expression data are the result of four biological replicates.

FIG. 7 shows tubulin immunofluorescence after treatment with deoxysappanone B and paclitaxel. C2C12 myotubes were treated with compounds for 48 hours and stained for microtubules using an anti-α-tubulin antibody (green) and nuclei using Hoechst 33342 (blue). Deoxysappanone B treatments: a, none, b, 10 nM, c, 100 nM, d, 1 μM, e, 10 μM. Paclitaxel treatments: f, none, g, 10 nM, h, 100 nM, i, 1 μM, j, 10 μM. Scale bar=50 μm.

FIG. 8 show measurements of the coupling between nuclear and mitochondrial OXPHOS gene expression. (a) A two-dimensional plot of the composite Z-scores for nuOXPHOS and mtOXPHOS expression is shown. (b) Row-normalized heat map displaying the top 15 compounds in each quadrant (I-IV). Heat map of nuOXPHOS and mtOXPHOS expression is shown along with ATP levels. (c) Real-time PCR validation of select compounds at the indicated doses, using Atp5a1 (nuOXPHOS) and mt-Co1 (mtOXPHOS) normalized to Hprt1 (internal control). Values indicate average fold change from mock-treated (DMSO) wells ±s.d. in four biological replicates.

FIG. 9 shows statin-induced mitochondrial toxicity. (a) Six of the HMG-CoA reductase inhibitors (statins) in clinical use are in the chemical screening collection. Composite Z-scores for cell viability, ATP generation, MTT activity, ΔΨm, ROS levels and gene expression are shown, where negative scores indicate a decrease in signal compared to mock-treated (DMSO) wells. The gray shading indicates scores that fall within the noise envelope. (b) A centroid statin score was generated by calculating the arithmetic means of the composite Z-scores for fluvastatin, lovastatin and simvastatin. The ten nearest neighbor clinically used drugs (amoxapine, cyclobenzaprine, propranolol, griseofulvin, pentamidine, paclitaxel, propafenone, ethaverine, trimeprazine and amitriptyline) were identified by calculating the root-mean-square distance of each performance vector to the profile of interest. (c) All six statins were tested in combination with three clinically used b-adrenergic blockers (propranolol, atenolol and metoprolol) for their effects on cellular ATP levels. Compound concentrations are indicated on each axis, and the grayscale intensity indicates the change in ATP levels (ranging from black, for no change, to medium gray, for a 50% decrease). Data represent the average of six independent replicates; coefficients of variation were all below 15%.

FIG. 10 shows the dose-response curves for statins and beta blockers for cellular ATP levels. a, The six statins in our collection were tested in doses as high as 40 μM for 48 hours before ATP levels were measured. The three mitochondrially active statins in the screening compendium are in gray (top to bottom: simvastatin, lovastatin, fluvastatin), while the other three are in black (pravastatin, rosuvastatin, atorvastatin). b, Three beta adrenergic antagonists (one nonselective and two beta₁-selective) were tested in doses as high as 40 μM for 48 hours and then ATP levels were measured. Black line, atenolol; light gray line, metoprolol, both selective antagonists; dark gray line, propranolol, a nonselective antagonist.

### SUMMARY OF THE INVENTION

The invention has been comtemplated such that all embodiments described herein, including those embodiments described under different aspects of the invention, can be combined with one another, where appropriate.

One aspect of the invention provides a method of treating or preventing a disorder characterized by mitochondrial dysfunction in a subject, the method comprising administering to the subject a therapeutically effective amount of a cytoskeleton modulator. In some embodiments, the cytoskeleton modulator is a microtubule modulator. In some embodiments, the microtubule modulator is a microtubule inhibitor. In some embodiments, the cytoskeleton modulator is a compound of Formula (I):

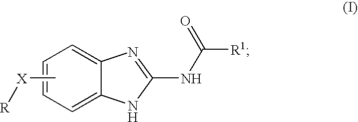

wherein R is selected from (C₁-C₄)alkyl, cycloalkyl having 3 to 6 carbon atoms, phenyl, halo-substituted phenyl in which halo in each occurrence is selected from Br, Cl, or F, (lower alkyl)-substituted phenyl, ((C₁-C₄)alkoxy)-substituted phenyl, and 2-thienyl; R¹ is selected from methyl and ethyl, X is selected from —S—, —C(O)—, —O—, —CH₂— and —S(O)— and the R—X— substituent is located at the 5(6)-position, or a salt thereof.

In some embodiments, the compound is mebendazole, a derivative, metabolite, or analog thereof. In some embodiments, the compound is mebendazole or a metabolite or analog thereof. In some embodiments, the subject is not afflicted with a worm infection. In some embodiments, the worm infection is a hookworm infection, a roundworm infection, a pinworm infection or a whipworm infection. In some embodiments, wherein the subject is not afflicted with diabetes. In some embodiments, the compound is nocodazole, a derivative, metabolite, or analog thereof.

In some embodiments, the compound is one of the following: albendazole, fenbendazole, oxfendazole, oxibendazole, methiazole, parbendazole, and any derivatives, metabolites, or analogs of the compounds listed.

In some embodiments, the cytoskeleton modulator is cytochalasin, a derivative, metabolite, or analog thereof. In some embodiments, the cytochalasin is selected from cytochalasin A, cytochalasin B, cytochalasin C, cytochalasin D, cytochalasin E, cytochalasin F, cytochalasin H, cytochalasin J, cytochalasin K, cytochalasin Q, cytochalasin R, epoxycytochalasin H and epoxycytochalasin J. In some embodiments, the cytochalasin is selected from cytochalasin E.

In some embodiments, the cytoskeleton modulator is a compound of Formula (II):

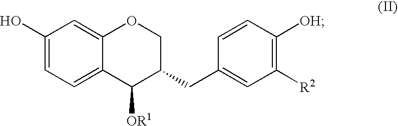

wherein R¹ is selected from H or methyl and R² is selected from H or hydroxy. In some embodiments, the cytoskeleton modulator is a compound selected from Formulas (III)-(VI):

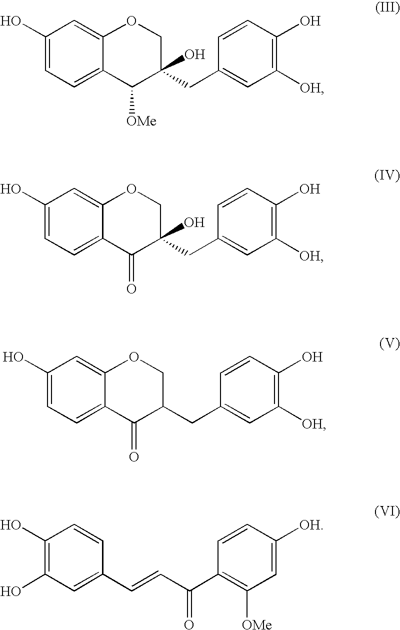

In some embodiments, the compound is deoxysappanone B, or a metabolite, or an analog thereof.

In some embodiments, the cytoskeleton modulator is a compound of Formula (VII):

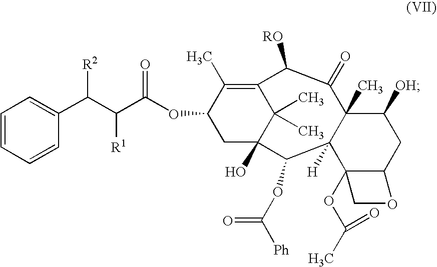

wherein, R is nitrogen or acetyl and one of R¹ and R² is hydroxy and the other is selected from t-butylcarbonylamino or benzoylamino.

In some embodiments, the compound is paclitaxel or a metabolite or analog thereof. In some embodiments, the compound is podofilox, a metabolite, analog, or salt thereof. In some embodiments, the compound is podophyllotoxin acetate.

In some embodiments, the cytoskeleton modulator is a compound of Formula (VIII):

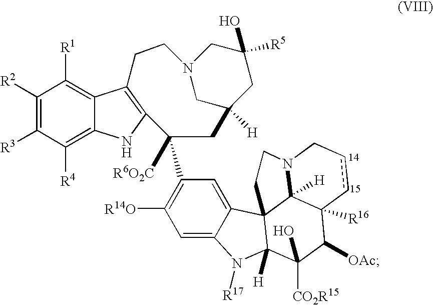

wherein R¹, R², R³ and R⁴ are independently selected from H, lower alkyl group, lower alkoxy group, halogen, lower perfluoroalkyl group, lower alkylthio group, hydroxy group, amino group, mono- or di-alkyl or acylamino group, lower alkyl or arylsulfonyloxy group, R⁵ is H, or a lower alkyl group or a substituted or non-substituted aryl group, R⁶ is an alkyl group of carbon number 4 or less, R¹⁴, R¹⁵ and R¹⁶ are an alkyl group of carbon number 4 or less, R¹⁷ is H or an alkyl group of carbon number 4 or less, and in between carbon 14 and carbon 15 is an unsaturated double bond or saturated bond.

In some embodiments, the compound is vinblastine or a metabolite or analog thereof.

In some embodiments, the compounds described herein can be used to increase glucose uptake in a cell.

In some embodiments, the mitochondrial dysfunction is characterized by reduced oxidative phosphorylation or increased generation of reactive oxygen species or both. In some embodiments, the disorder is diabetes or glucose intolerance. In some embodiments, the disorder is, obesity, cardiac myopathy, premature aging, coronary atherosclerotic heart disease, diabetes mellitus, Alzheimer's Disease, Parkinson's Disease, Huntington's disease, dystonia, Leber's hereditary optic neuropathy (LHON), schizophrenia, myodegenerative disorders such as “mitochondrial encephalopathy, lactic acidosis, and stroke” (MELAS). and “myoclonic epilepsy ragged red fiber syndrome” (MERRF), NARP (Neuropathy; Ataxia; Retinitis Pigmentosa), MNGIE (Myopathy and external opthalmoplegia, neuropathy; gastro-intestinal encephalopathy, Kearns-Sayre disease, Pearson's Syndrome, PEO (Progressive External Opthalmoplegia), congenital muscular dystrophy with mitochondrial structural abnormalities, Wolfram syndrome, Diabetes Insipidus, Diabetes Mellitus, Optic Atrophy Deafness, Leigh's Syndrome, fatal infantile myopathy with severe mitochondrial DNA (mtDNA) depletion, benign “later-onset” myopathy with moderate reduction in mtDNA, dystonia, medium chain acyl-CoA dehydrogenase deficiency, arthritis, and mitochondrial diabetes and deafness (MIDD), mitochondrial DNA depletion syndrome.

In some embodiments, the subject is not afflicted with cancer.

In some embodiments, the disorder is obesity. In some embodiments, the disorder is diabetes. In some embodiments, the diabetes is type 2 diabetes mellitus. In some embodiments, the disorder is glucose intolerance. In some embodiments, the subject has elevated gluconeogenesis. In some embodiments, the disorder is premature aging. In some embodiments, the disorder is a neurodegenerative disorder. In some embodiments, the neurodegenerative disorder is characterized by neuronal cell death. In some embodiments, the neurodegenerative disorder is Parkinson disease, amyotrophic lateral sclerosis (ALS), Alzheimer's disease, Huntington's disease or Freidreich's ataxia.

In some embodiments, the disorder is selected from Familial British Dimentia, Finnish-type Familial Amyloidoses, Frontotemporal Dementia, Senile Systemic Amyloidosis, Familial Amyloid Polyneuropathy, Transmissible Spongiform Encephalopathie, Gertsmann-Strausseler-Scheinker Syndrome, Fatal Familial Insomnia, Huntington's Chorea, Kuru, Familial amyloid polyneuropathy, Creutzfeldt Jakob, Scrapie, and Bovine Spongiform Encephalopathy.

In some embodiments, the disorder is an mtDNA-associated disease. In some embodiments, the mt-DNA associated disease is MERRF, MELAS, LHON, MILASA, MILS, PEO or KSS.

In some embodiments, the disorder is a mitochondrial encephalomyopathy due to nuclear gene mutations. In some embodiments, the encephalomyopathy is Leigh syndrome French Canadian variety, mtDNA depletion syndromes, Barth syndrome and Wilson's disease. In some embodiments, the disorder is a congenital mitochondrial disorder.

In some embodiments, the compound is cytochalasin E or a metabolite or analog thereof. In some embodiments, the compound is deoxysappanone or a metabolite, analog or derivative thereof.

In some embodiments, the deoxysappanone is selected from deoxysappanone (B) 7,3′-dimethyl ether, sappanone (A) trimethyl ether, or 3-deshydroxysappanol trimethyl ether. In some embodiments, the subject is not afflicted with diabetes. In some embodiments, the compound is nocodazole or a metabolite or analog thereof. In some embodiments, the compound is paclitaxel or a metabolite or analog thereof. In some embodiments, the compound is podofilox or a metabolite or analog thereof. In some embodiments, the compound is podophyllotoxin acetate or a metabolite or analog thereof. In some embodiments, the compound is vinblastine or a metabolite or analog thereof.

In some embodiments, the disorder is cardiovascular disease. In some embodiments, the disorder is cardiomyopathy.

In some embodiments, the method of treating or preventing a disorder characterized by mitochondrial dysfunction in a subject further comprises administering to the subject one or more agents selected from sulfonylureas, non-sulfonylurea secretagogues, insulin, insulin analogs, glucagon-like peptides, exendin-4 polypeptides, beta 3 adrenoceptor agonists, PPAR agonists, dipeptidyl peptidase IV inhibitors, biguanides, alpha-glucosidase inhibitors, immunomodulators, statins and statin-containing combinations, angiotensin converting enzyme inhibitors, adeno sine A1 receptor agonists, adenosine A2 receptor agonists, aldosterone antagonists, alpha 1 adrenoceptor antagonists, alpha 2 adrenoceptor agonists, alpha 2 adrenoceptor agonists, angiotensin receptor antagonists, antioxidants, ATPase inhibitors, atrial peptide agonists, beta adrenoceptor antagonists, calcium channel agonists, calcium channel antagonists, diuretics, dopamine D1 receptor agonists, endopeptidase inhibitors, endothelin receptor antagonists, guanylate cyclase stimulants, phosphodiesterase V inhibitors, protein kinase inhibitors, Cdc2 kinase inhibitors, renin inhibitors, thromboxane synthase inhibitors, vasopeptidase inhibitors, vasopressin I antagonists, vasopressin 2 antagonists, angiogenesis inhibitors, advanced glycation end product inhibitors, bile acid binding agents, bile acid transport inhibitors, bone formation stimulants, apolipoprotein A1 agonists, DNA topoisomerase inhibitors, cholesterol absorption inhibitors, cholesterol antagonists, cholesteryl ester transfer protein antagonists, cytokine synthesis inhibitors, DNA polymerase inhibitors, dopamine D2 receptor agonists, endothelin receptor antagonists, growth hormone antagonists, insulin sensitizers, lipase inhibitors, lipid peroxidation inhibitors, lipoprotein A antagonists, microsomal transport protein inhibitors, microsomal triglyceride transfer protein inhibitors, nitric oxide synthase inhibitors, oxidizing agents, phospholipase A2 inhibitors, radical formation agonists, platelet aggregation antagonists, prostaglandin synthase stimulants, reverse cholesterol transport activators, rho kinase inhibitors, selective estrogen receptor modulators, squalene epoxidase inhibitors, squalene synthase inhibitors, thromboxane A2 antagonists, amylin agonists, cannabinoid receptor antagonists, cholecystokinin A agonists, corticotropin-releasing factor agonists, dopamine uptake inhibitors, G protein-coupled receptor modulators, glutamate antagonists, glucagon-like peptide-1 agonists, insulin sensitizers, lipase inhibitors, melanin-concentrating hormone receptor antagonists, nerve growth factor agonists, neuropeptide Y agonists, neuropeptide Y antagonists, SNRIs, protein tyrosine phosphatase inhibitors, serotonin 2C receptor agonists, bezafibrate, diflunisal, or cinnamic acid.

In some embodiments, said sulfonylurea is selected from the group consisting of acetohexamide, chlorpropamide, tolazamide, tolbutamide, glimepiride, glipizide, and glyburide. In some embodiments, said non-sulfonylurea secretagogue is nateglinide or repaglinide. In some embodiments, said insulin analog is selected from the group consisting of insulin lispro, insulin aspart, insulin glarginine, NPH, lente insulin, ultralente insulin, humulin, and novolin. In some embodiments, said PPAR agonist is selected from the group consisting of balaglitazone, troglitazone, pioglitazone, ciglitazone, englitazone, rosiglitazone, darglitazone, englitazone, netoglitazone, KRP-297, JTT-501, NC-2100, NIP-223, MCC-555, L-764486, CS-011, G1262570, GW347845, and FK614. In some embodiments, said biguanide is metformin or metformin/glyburide. In some embodiments, said alpha-glucosidase inhibitor is acarbose or miglitol. In some embodiments, said immunomodulator is a corticosteroid, cyclophosphamide, or NsIDI. In some embodiments, said angiotensin converting enzyme (ACE) inhibitor is selected from the group consisting of benazepril, captopril, cilazapril, enalapril, enalaprilat, fosinopril, lisinopril, moexipril, perindopril, quinapril, ramipril, and trandolapril. In some embodiments, said angiotensin II receptor blocker is selected from the group consisting of candesartan, eprosartan, irbesarten, losartin, telmisartan, and valsartan. In some embodiments, said antioxidant is selected from the group consisting of nicotinamide, vitamin E, probucol, MDL29311, and U78518F. In some embodiments, said exendin 4 is AC2993. In some embodiments, said glucagon-like peptide is GLP-1.

In another aspect of the invention, methods are provided for identifying compounds that enhance mitochondrial function, comprising (i) assaying for the effect of one or more compounds on (a) OXPHOS gene expression and (b) mitochondrial function; and (ii) correlating the effect with a compound's enhancement of mitochondrial function, wherein an increase in OXPHOS gene expression and an increase in mitochondrial function is indicative of a compound that enhances mitochondrial function. In some embodiments, the assay is performed on murine myotubes. In some embodiments, mitochondrial function is assayed by measuring reactive oxygen species (ROS). In some embodiments, an increase in OXPHOS gene expression and a decrease in ROS is indicative of a compound that enhances mitochondrial function. In some embodiments, the method further comprises assaying for the effect of one or more compounds on (c) cell viability, and wherein the lack of a decrease on cell viability is indicative of a compound that enhances mitochondrial function. In some embodiments, cell viability is measured using calcein dye. In some embodiments, comprises assaying for the effect of one or more compounds on one or more of the following: cellular dehydrogenase activity; mitochondrial membrane potential; cellular ATP; and cytochrome c protein.

In some embodiments, OXPHOS gene expression is measured using a gene expression-based high-throughput screening (GE-HTS) assay. In some embodiments, OXPHOS gene expression comprises the expression of the following genes: (a) Mt-Atp6 (Entrez GeneID numbers 17705 or 4508), (b) Mt-Atp8 (Entrez GeneID numbers 17706 or 4509), (c) Mt-Co1 (Entrez GeneID numbers 17708 or 4512), (d) Mt-Co2 (Entrez GeneID numbers 17709 or 4513), (e) Mt-Co3 (Entrez GeneID numbers 17710 or 4514), (f) Mt-Cytb (Entrez GeneID number 17711 or 4519), (g) Mt-Nd1 (Entrez GeneID numbers 17716 or 4535), (h) Mt-Nd2 (Entrez GeneID numbers 17717 or 4536), (i) Mt-Nd3 (Entrez GeneID numbers 17718 or 4537), (j) Mt-Nd4 (Entrez GeneID numbers 17719 or 4538), (k) Mt-Nd41 (Entrez GeneID numbers 17720 or 4539), (l) Mt-Nd5 (Entrez GeneID numbers 17721 or 4540), (m) Mt-Nd6 (Entrez GeneID numbers 17722 or 4541), (n) Atp5a1 (Entrez GeneID numbers 11946 or 498), (o) Atp5c1 (Entrez GeneID numbers 11949 or 509), (p) Atp5o (Entrez GeneID numbers 28080 or 539), (q) Cox5b (Entrez GeneID numbers 12859 or 1329), (r) Cox7a2 (Entrez GeneID numbers 12866 or 1347), (s) Cyc1 (Entrez GeneID numbers 66445 or 1537), (t) Hspc051 (Entrez GeneID number 66152 or 29796), (u) Ndufa5 (Entrez GeneID numbers 68202 or 4698), (v) Ndufb5 (Entrez GeneID numbers 66046 or 4711), (w) Sdhd (Entrez GeneID numbers 66925 or 6392), (x) Uqcrb (Entrez GeneID numbers 67530 or 7381), and (y) Uqcrc1 (Entrez GeneID numbers 22273 or 7384)

In some embodiments, the assays are performed in a multi-well plate format. In some embodiments, the one or more compounds comprise a library of compounds.

In another aspect of the invention, methods are provided for identifying compounds for treating a disorder characterized by mitochondrial dysfunction in a subject comprising (i) assaying for the effect of one or more compounds on (a) OXPHOS gene expression and (b) mitochondrial function; and (ii) correlating the effect with a compound's ability to treat said disorder, wherein an increase in OXPHOS gene expression and an increase in mitochondrial function is indicative of a compound useful for treating said disorder. In some embodiments, mitochondrial function is assayed by measuring reactive oxygen species (ROS). In some embodiments, an increase in OXPHOS gene expression and a decrease in ROS is indicative of a compound that enhances mitochondrial function.

In some embodiments, the method further comprises assaying for the effect of one or more compounds on cell viability, and wherein the lack of a decrease on cell viability is indicative of a compound that enhances mitochondrial function. In some embodiments, cell viability is measured using calcein dye. In some embodiments, the mitochondrial function is assayed by measuring reactive oxygen species (ROS) and further comprises assaying for the effect of one or more compounds on one or more of the following: cellular dehydrogenase activity; mitochondrial membrane potential; cellular ATP; and cytochrome c protein, wherein an increase in cellular dehydrogenase activity, an increase in mitochondrial membrane potential; an increase cellular ATP; and an increase in cytochrome c protein is indicative of a compound that enhances mitochondrial function.

In some embodiments, OXPHOS gene expression is measured using a gene expression-based high-throughput screening (GE-HTS) assay. In some embodiments, OXPHOS gene expression comprises the expression of the following genes: (a) Mt-Atp6 (Entrez GeneID numbers 17705 or 4508), (b) Mt-Atp8 (Entrez GeneID numbers 17706 or 4509), (c) Mt-Co1 (Entrez GeneID numbers 17708 or 4512), (d) Mt-Co2 (Entrez GeneID numbers 17709 or 4513), (e) Mt-Co3 (Entrez GeneID numbers 17710 or 4514), (f) Mt-Cytb (Entrez GeneID number 17711 or 4519), (g) Mt-Nd1 (Entrez GeneID numbers 17716 or 4535), (h) Mt-Nd2 (Entrez GeneID numbers 17717 or 4536), (i) Mt-Nd3 (Entrez GeneID numbers 17718 or 4537), (j) Mt-Nd4 (Entrez GeneID numbers 17719 or 4538), (k) Mt-Nd41 (Entrez GeneID numbers 17720 or 4539), (l) Mt-Nd5 (Entrez GeneID numbers 17721 or 4540), (In) Mt-Nd6 (Entrez GeneID numbers 17722 or 4541), (n) Atp5a1 (Entrez GeneID numbers 11946 or 498), (o) Atp5c1 (Entrez GeneID numbers 11949 or 509), (p) Atp5o (Entrez GeneID numbers 28080 or 539), (q) Cox5b (Entrez GeneID numbers 12859 or 1329), (r) Cox7a2 (Entrez GeneID numbers 12866 or 1347), (s) Cyc1 (Entrez GeneID numbers 66445 or 1537), (t) Hspc051 (Entrez GeneID number 66152 or 29796), (u) Ndufa5 (Entrez GeneID numbers 68202 or 4698), (v) Ndufb5 (Entrez GeneID numbers 66046 or 4711), (w) Sdhd (Entrez GeneID numbers 66925 or 6392), (x) Uqcrb (Entrez GeneID numbers 67530 or 7381), and (y) Uqcrc1 (Entrez GeneID numbers 22273 or 7384)

In some embodiments, the assays are performed in a multi-well plate format. In some embodiments, the one or more compounds comprise a library of compounds.

In some embodiments, the mitochondrial dysfunction is characterized by reduced oxidative phosphorylation or increased generation of reactive oxygen species or both. In some embodiments, the disorder is type II diabetes. In some embodiments, the disorder is a neurodegenerative disease selected from Parkinson's or Huntington's disease. In some embodiments, the disorder is cardiovascular disease. In some embodiments, the disorder is cardiomyopathy.

In another aspect of the invention, methods are provided for determining compounds that are contraindicated in a subject, comprising (i) assaying for the effect of one or more compounds on (a) cellular dehydrogenase activity and (b) cell viability; and (ii) correlating the effect with contraindication of a compound, wherein a decrease in cellular dehydrogenase activity absent a decrease in cell viability indicates that the compound is contraindicated for said subjects.

In some embodiments, said subject is afflicted with a disorder characterized by mitochondrial dysfunction. In some embodiments, the method for determining compounds that are contraindicated in a subject further comprises assaying for the effect of one or more compounds on one or more of the following: OXPHOS gene expression; mitochondrial membrane potential; cellular ATP; reactive oxygen species (ROS), and cytochrome c protein, wherein an increase in OXPHOS gene expression, an increase in mitochondrial membrane potential; an increase in cellular ATP; an increase in ROS, and an increase in cytochrome c protein is indicative of a compound that enhances mitochondrial function. In some embodiments, mitochondrial function is assayed by measuring reactive oxygen species (ROS).

In some embodiments, an increase in OXPHOS gene expression and a decrease in ROS is indicative of a compound that enhances mitochondrial function. In some embodiments, cell viability is measured using calcein dye. In some embodiments, OXPHOS gene expression is measured using a gene expression-based high-throughput screening (GE-HTS) assay. In some embodiments, OXPHOS gene expression comprises the expression of the following genes: (a) Mt-Atp6 (Entrez GeneID numbers 17705 or 4508), (b) Mt-Atp8 (Entrez GeneID numbers 17706 or 4509), (c) Mt-Co1 (Entrez GeneID numbers 17708 or 4512), (d) Mt-Co2 (Entrez GeneID numbers 17709 or 4513), (e) Mt-Co3 (Entrez GeneID numbers 17710 or 4514), (f) Mt-Cytb (Entrez GeneID number 17711 or 4519), (g) Mt-Nd1 (Entrez GeneID numbers 17716 or 4535), (h) Mt-Nd2 (Entrez GeneID numbers 17717 or 4536), (i) Mt-Nd3 (Entrez GeneID numbers 17718 or 4537), (j) Mt-Nd4 (Entrez GeneID numbers 17719 or 4538), (k) Mt-Nd41 (Entrez GeneID numbers 17720 or 4539), (l) Mt-Nd5 (Entrez GeneID numbers 17721 or 4540), (m) Mt-Nd6 (Entrez GeneID numbers 17722 or 4541), (n) Atp5a1 (Entrez GeneID numbers 11946 or 498), (o) Atp5c1 (Entrez GeneID numbers 11949 or 509), (p) Atp5o (Entrez GeneID numbers 28080 or 539), (q) Cox5b (Entrez GeneID numbers 12859 or 1329), (r) Cox7a2 (Entrez GeneID numbers 12866 or 1347), (s) Cyc1 (Entrez GeneID numbers 66445 or 1537), (t) Hspc051 (Entrez GeneID number 66152 or 29796), (u) Ndufa5 (Entrez GeneID numbers 68202 or 4698), (v) Ndufb5 (Entrez GeneID numbers 66046 or 4711), (w) Sdhd (Entrez GeneID numbers 66925 or 6392), (x) Uqcrb (Entrez GeneID numbers 67530 or 7381), and (y) Uqcrc1 (Entrez GeneID numbers 22273 or 7384)

In some embodiments, the assays are performed in a multi-well plate format. In some embodiments, the one or more compounds comprise a library of compounds.

In some embodiments, the mitochondrial dysfunction is characterized by reduced oxidative phosphorylation or increased generation of reactive oxygen species or both. In some embodiments, the disorder is type II diabetes. In some embodiments, the disorder is a neurodegenerative disease selected from Parkinson's or Huntington's disease. In some embodiments, the disorder is cardiovascular disease. In some embodiments, the disorder is cardiomyopathy.

In another aspect of the invention, methods are provided for determining two or more compounds that are contraindicated for joint administration to a subject comprising (i) assaying for the effect of two or more compounds on (a) cellular dehydrogenase activity and (b) cell viability; and (ii) correlating the effect with contraindication of joint administration, wherein two or more compounds that each decrease cellular dehydrogenase activity absent a decrease in cell viability indicates that the two or more compounds are contraindicated when jointly administered to a subject. In some embodiments, the subject is afflicted with a disorder characterized by mitochondrial dysfunction. In some embodiments, the methods of determining two or more compounds that are contraindicated for joint administration to a subject further comprises assaying for the effect of one or more compounds on one or more of the following: OXPHOS gene expression; mitochondrial membrane potential; cellular ATP; reactive oxygen species (ROS), and cytochrome c protein, wherein an increase in OXPHOS gene expression, an increase in mitochondrial membrane potential; an increase in cellular ATP; an increase in ROS, and an increase in cytochrome c protein is indicative of a compound that enhances mitochondrial function. In some embodiments, mitochondrial function is assayed by measuring reactive oxygen species (ROS).

In some embodiments, an increase in OXPHOS gene expression and a decrease in ROS is indicative of a compound that enhances mitochondrial function. In some embodiments, cell viability is measured using calcein dye. In some embodiments, OXPHOS gene expression is measured using a gene expression-based high-throughput screening (GE-HTS) assay. In some embodiments, OXPHOS gene expression comprises the expression of the following genes: (a) Mt-Atp6 (Entrez GeneID numbers 17705 or 4508), (b) Mt-Atp8 (Entrez GeneID numbers 17706 or 4509), (c) Mt-Co1 (Entrez GeneID numbers 17708 or 4512), (d) Mt-Co2 (Entrez GeneID numbers 17709 or 4513), (e) Mt-Co3 (Entrez GeneID numbers 17710 or 4514), (f) Mt-Cytb (Entrez GeneID number 17711 or 4519), (g) Mt-Nd1 (Entrez GeneID numbers 17716 or 4535), (h) Mt-Nd2 (Entrez GeneID numbers 17717 or 4536), (i) Mt-Nd3 (Entrez GeneID numbers 17718 or 4537), (j) Mt-Nd4 (Entrez GeneID numbers 17719 or 4538), (k) Mt-Nd41 (Entrez GeneID numbers 17720 or 4539), (l) Mt-Nd5 (Entrez GeneID numbers 17721 or 4540), (m) Mt-Nd6 (Entrez GeneID numbers 17722 or 4541), (n) Atp5a1 (Entrez GeneID numbers 11946 or 498), (o) Atp5c1 (Entrez GeneID numbers 11949 or 509), (p) Atp5o (Entrez GeneID numbers 28080 or 539), (q) Cox5b (Entrez GeneID numbers 12859 or 1329), (r) Cox7a2 (Entrez GeneID numbers 12866 or 1347), (s) Cyc1 (Entrez GeneID numbers 66445 or 1537), (t) Hspc051 (Entrez GeneID number 66152 or 29796), (u) Ndufa5 (Entrez GeneID numbers 68202 or 4698), (v) Ndufb5 (Entrez GeneID numbers 66046 or 4711), (w) Sdhd (Entrez GeneID numbers 66925 or 6392), (x) Uqcrb (Entrez GeneID numbers 67530 or 7381), and (y) Uqcrc1 (Entrez GeneID numbers 22273 or 7384)

In some embodiments, the assays are performed in a multi-well plate format. In some embodiments, the one or more compounds comprise a library of compounds. In some embodiments, the mitochondrial dysfunction is characterized by reduced oxidative phosphorylation or increased generation of reactive oxygen species or both. In some embodiments, the disorder is type II diabetes. In some embodiments, the disorder is a neurodegenerative disease selected from Parkinson's or Huntington's disease. In some embodiments, wherein the disorder is cardiovascular disease. In some embodiments, the disorder is cardiomyopathy.

In another aspect of the invention, a kit for determining OXPHOS gene expression is provided, comprising a set of primer pairs, each pair amplifying an OXPHOS gene selected from a group consisting of the following: (a) Mt-Atp6 (Entrez GeneID numbers 17705 or 4508), (b) Mt-Atp8 (Entrez GeneID numbers 17706 or 4509), (c) Mt-Co1 (Entrez GeneID numbers 17708 or 4512), (d) Mt-Co2 (Entrez GeneID numbers 17709 or 4513), (e) Mt-Co3 (Entrez GeneID numbers 17710 or 4514), (f) Mt-Cytb (Entrez GeneID number 17711 or 4519), (g) Mt-Nd1 (Entrez GeneID numbers 17716 or 4535), (h) Mt-Nd2 (Entrez GeneID numbers 17717 or 4536), (i) Mt-Nd3 (Entrez GeneID numbers 17718 or 4537), (o) Mt-Nd4 (Entrez GeneID numbers 17719 or 4538), (k) Mt-Nd41 (Entrez GeneID numbers 17720 or 4539), (l) Mt-Nd5 (Entrez GeneID numbers 17721 or 4540), (m) Mt-Nd6 (Entrez GeneID numbers 17722 or 4541), (n) Atp5a1 (Entrez GeneID numbers 11946 or 498), (o) Atp5c1 (Entrez GeneID numbers 11949 or 509), (p) Atp5o (Entrez GeneID numbers 28080 or 539), (q) Cox5b (Entrez GeneID numbers 12859 or 1329), (r) Cox7a2 (Entrez GeneID numbers 12866 or 1347), (s) Cyc1 (Entrez GeneID numbers 66445 or 1537), (t) Hspc051 (Entrez GeneID number 66152 or 29796), (u) Ndufa5 (Entrez GeneID numbers 68202 or 4698), (v) Ndufb5 (Entrez GeneID numbers 66046 or 4711), (w) Sdhd (Entrez GeneID numbers 66925 or 6392), (x) Uqcrb (Entrez GeneID numbers 67530 or 7381), and (y) Uqcrc1 (Entrez GeneID numbers 22273 or 7384).

In some embodiments, the first primer pair comprises a first primer comprising the nucleotide sequence of SEQ ID NO: 1 and a second primer comprising the nucleotide sequence of SEQ ID NO: 2; the second primer pair comprises a first primer comprising the nucleotide sequence of SEQ ID NO: 3 and a second primer comprising the nucleotide sequence of SEQ ID NO: 4; the third primer pair comprises a first primer comprising the nucleotide sequence of SEQ ID NO: 5 and a second primer comprising the nucleotide sequence of SEQ ID NO: 6; the fourth primer pair comprises a first primer comprising the nucleotide sequence of SEQ ID NO: 7 and a second primer comprising the nucleotide sequence of SEQ ID NO: 8; the fifth primer pair comprises a first primer comprising the nucleotide sequence of SEQ ID NO: 9 and a second primer comprising the nucleotide sequence of SEQ ID NO: 10, the sixth primer pair comprises a first primer comprising the nucleotide sequence of SEQ ID NO: 11 and a second primer comprising the nucleotide sequence of SEQ ID NO: 12, the seventh primer pair comprises a first primer comprising the nucleotide sequence of SEQ ID NO: 13 and a second primer comprising the nucleotide sequence of SEQ ID NO: 14, the eighth primer pair comprises a first primer comprising the nucleotide sequence of SEQ ID NO: 15 and a second primer comprising the nucleotide sequence of SEQ ID NO: 16, the ninth primer pair comprises a first primer comprising the nucleotide sequence of SEQ ID NO: 17 and a second primer comprising the nucleotide sequence of SEQ ID NO: 18, the tenth primer pair comprises a first primer comprising the nucleotide sequence of SEQ ID NO: 19 and a second primer comprising the nucleotide sequence of SEQ ID NO: 20, the eleventh primer pair comprises a first primer comprising the nucleotide sequence of SEQ ID NO: 21 and a second primer comprising the nucleotide sequence of SEQ ID NO: 22, the twelfth primer pair comprises a first primer comprising the nucleotide sequence of SEQ ID NO: 23 and a second primer comprising the nucleotide sequence of SEQ ID NO: 24, the thirteenth primer pair comprises a first primer comprising the nucleotide sequence of SEQ ID NO: 25 and a second primer comprising the nucleotide sequence of SEQ ID NO: 26, the fourteenth primer pair comprises a first primer comprising the nucleotide sequence of SEQ ID NO: 27 and a second primer comprising the nucleotide sequence of SEQ ID NO: 28, the fifteenth primer pair comprises a first primer comprising the nucleotide sequence of SEQ ID NO: 29 and a second primer comprising the nucleotide sequence of SEQ ID NO: 30, the sixteenth primer pair comprises a first primer comprising the nucleotide sequence of SEQ ID NO: 31 and a second primer comprising the nucleotide sequence of SEQ ID NO: 32, the seventeenth primer pair comprises a first primer comprising the nucleotide sequence of SEQ ID NO: 33 and a second primer comprising the nucleotide sequence of SEQ ID NO: 34, the eighteenth primer pair comprises a first primer comprising the nucleotide sequence of SEQ ID NO: 35 and a second primer comprising the nucleotide sequence of SEQ ID NO: 36, the nineteenth primer pair comprises a first primer comprising the nucleotide sequence of SEQ ID NO: 37 and a second primer comprising the nucleotide sequence of SEQ ID NO: 38, the twentieth primer pair comprises a first primer comprising the nucleotide sequence of SEQ ID NO: 39 and a second primer comprising the nucleotide sequence of SEQ ID NO: 40, the twenty-first primer pair comprises a first primer comprising the nucleotide sequence of SEQ ID NO: 41 and a second primer comprising the nucleotide sequence of SEQ ID NO: 42, the twenty-second primer pair comprises a first primer comprising the nucleotide sequence of SEQ ID NO: 43 and a second primer comprising the nucleotide sequence of SEQ ID NO: 44, the twenty-third primer pair comprises a first primer comprising the nucleotide sequence of SEQ ID NO: 45 and a second primer comprising the nucleotide sequence of SEQ ID NO: 46, the twenty-fourth primer pair comprises a first primer comprising the nucleotide sequence of SEQ ID NO: 47 and a second primer comprising the nucleotide sequence of SEQ ID NO: 48, the twenty-fifth primer pair comprises a first primer comprising the nucleotide sequence of SEQ ID NO: 49 and a second primer comprising the nucleotide sequence of SEQ ID NO: 50.

In some embodiments, the kit comprises at least one primer pair that amplifies a gene showing little or no upregulation by PGC-1a. In some embodiments, at least one primer pair amplifies a gene selected from (a) Actb (Entrez GeneID 11461), (b) Aamp (Entrez GeneID 227290), (c) Cenpb (Entrez GeneID 12616), (d) Eefla1 (Entrez GeneID 13627), (e) Jund (Entrez GeneID 16478), (f) Lsp1 (Entrez GeneID 16985), (g) Rps2 (Entrez GeneID 16898), and (h) Rps27a (Entrez GeneID 78294). In some embodiments, the first primer pair comprises a first primer comprising the nucleotide sequence of SEQ ID NO: 51 and a second primer comprising the nucleotide sequence of SEQ ID NO: 52; the second primer pair comprises a first primer comprising the nucleotide sequence of SEQ ID NO: 53 and a second primer comprising the nucleotide sequence of SEQ ID NO: 54; the third primer pair comprises a first primer comprising the nucleotide sequence of SEQ ID NO: 55 and a second primer comprising the nucleotide sequence of SEQ ID NO: 56; the fourth primer pair comprises a first primer comprising the nucleotide sequence of SEQ ID NO: 57 and a second primer comprising the nucleotide sequence of SEQ ID NO: 58; the fifth primer pair comprises a first primer comprising the nucleotide sequence of SEQ ID NO: 59 and a second primer comprising the nucleotide sequence of SEQ ID NO: 60, the sixth primer pair comprises a first primer comprising the nucleotide sequence of SEQ ID NO: 61 and a second primer comprising the nucleotide sequence of SEQ ID NO: 62, the seventh primer pair comprises a first primer comprising the nucleotide sequence of SEQ ID NO: 63 and a second primer comprising the nucleotide sequence of SEQ ID NO: 64, the eighth primer pair comprises a first primer comprising the nucleotide sequence of SEQ ID NO: 65 and a second primer 66.

In some embodiments, the kit further comprises at least one primer pair that amplifies a genes that is down-regulated by PGC-1α. In some embodiments, at least one primer pair amplifies a gene selected from (a) Cyb5r3 (Entrez Gene ID 109754), and (b) Fh11 (Entrez Gene ID 14199).

In some embodiments, the first primer pair comprises a first primer comprising the nucleotide sequence of SEQ ID NO: 67 and a second primer comprising the nucleotide sequence of SEQ ID NO: 68; the second primer pair comprises a first primer comprising the nucleotide sequence of SEQ ID NO: 69 and a second primer comprising the nucleotide sequence of SEQ ID NO: 70.

In some embodiments, the kit further comprises reagents for amplifying DNA, wherein the reagents include a DNA polymerase.

In other embodiments, the kit comprises a plurality of primer pairs wherein each primer pair comprises a first nucleic acid sequence and a second nucleic acid sequence, which first nucleic acid sequence hybridizes under stringent conditions to a first strand of a target sequence, and which second nucleic acid sequence hybridizes under stringent conditions to a second strand of a target sequence, wherein the target sequence is selected from a group consisting of the following: (a) Mt-Atp6, (b) Mt-Atp8, (c) Mt-Co1, (d) Mt-Co2, (e) Mt-Co3, (f) Mt-Cytb, (g) Mt-Nd1, (h) Mt-Nd2, (i) Mt-Nd3, (j) Mt-Nd4, (k) Mt-Nd41, (l) Mt-Nd5, (m) Mt-Nd61, (n) Atp5a1, (o) Atp5c1, (p) Atp5o, (q) Cox5b, (r) Cox7a2, (s) Cyc1, (t) Hspc051, (u) Ndufa5, (v) Ndufb5, (w) Sdhd, (x) Uqcrb, and (y) Uqcrc1.

In some embodiments, primers in the primer pair hybridize under stringent conditions to the 3′ ends of the strands of the target sequence.

In some embodiments, the target sequence may be the entire gene or any appropriate region thereof.

In some embodiments, the kit comprises a first nucleic acid and/or the second nucleic acid further comprises a tag sequence. In some embodiments, the tag sequence is covalently linked to the 5′ end of the first and/or the second nucleic acid.

In further embodiments, the kit comprises a tag sequence that does not hybridize to the target sequence.

In additional embodiments, the kit comprises tag sequences, wherein said tag sequences are selected from the following: (a) SEQ ID NO:71, (b) SEQ ID NO:72, (c) SEQ ID NO:73, (d) SEQ ID NO:74, (e) SEQ ID NO:75, (f) SEQ ID NO:76, (g) SEQ ID NO:77, (h) SEQ ID NO:78, (i) SEQ ID NO:79, (j) SEQ ID NO:80, (k) SEQ ID NO:81, (l) SEQ ID NO:82, (m) SEQ ID NO:83, (n) SEQ ID NO:84, (o) SEQ ID NO:85, (p) SEQ ID NO:86, (q) SEQ ID NO:87, (r) SEQ ID NO:88, (s) SEQ ID NO:89, (t) SEQ ID NO:90, (u) SEQ ID NO:91, (v) SEQ ID NO:92, (w) SEQ ID NO:93, (x) SEQ ID NO:94, (y) SEQ ID NO:95, (z) SEQ ID NO:96, (aa) SEQ ID NO:97, (bb) SEQ ID NO:98, (cc) SEQ ID NO:99, (dd) SEQ ID NO:100, (ee) SEQ ID NO:101, (ff) SEQ ID NO:102, (gg) SEQ ID NO:103, (hh) SEQ ID NO:104, (ii) SEQ ID NO:105.

In other embodiments, the kit comprises a plurality of primer pairs, wherein each nucleic acid in the primer pair comprises a nucleic acid sequence that hybridizes under stringent conditions to the target sequence, is covalently linked to a tag sequence and/or an additional nucleic acid sequence. In some embodiments, primers in said primer pair hybridize under stringent conditions to the 3′ ends of the strands of the target sequence. In some embodiments, the additional nucleic acid sequence is not represented in either the target sequence or the tag sequence. In additional embodiments, the additional nucleic acid sequence comprises the binding site for a universal primer such as T3 or T7.

In some embodiments, the tag sequences comprise any one of SEQ ID NOs 71-105, listed in Table 9. In some embodiments, the additional nucleic acid sequence comprises the binding site for a universal primer, such as, but not limited to, T3 or T7. In some embodiments, the universal primers comprise either one of SEQ ID NOs 106-107, listed in Table 9. The primer sequences set forth herein may be combined with any one of the tag sequences provided herein or known in the art. For example, SEQ ID 108 is a primer sequence comprising the tag of SEQ ID NO: 76 linked to the universal primer of SEQ ID NO: 106 and further linked to the target specific primer of SEQ ID NO: 1. Other exemplary combinations are listed in Table 10 (SEQ ID NO: 108-176), and represent a subset of possible combinations.

In one aspect of the invention, methods are provided for detecting levels of at least 2 OXPHOS genes, comprising: (1) providing one or more target sequences selected from the following: (a) Mt-Atp6, (b) Mt-Atp8, (c) Mt-Co1, (d) Mt-Co2, (e) Mt-Co3, (f) Mt-Cytb, (g) Mt-Nd1, (h) Mt-Nd2, (i) Mt-Nd3, (j) Mt-Nd4, (k) Mt-Nd41, (l) Mt-Nd5, (m) Mt-Nd61, (n) Atp5a1, (o) Atp5c1, (p) Atp5o, (q) Cox5b, (r) Cox7a2, (s) Cyc1, (t) Hspc051, (u) Ndufa5, (v) Ndufb5, (w) Sdhd, (x) Uqcrb, and (y) Uqcrc1, (2) providing the plurality of primers that hybridize under stringent conditions to a target sequence from step (1), (3) amplifying target sequences using primers, (4) amplifying the sequences of step (3) using 2 nucleic acid sequences that are complementary to at least 1 portion of the primers of step (2), wherein one nucleic acid sequence is linked to a binding moiety, and one nucleic acid sequence is phosphorylated, and (5) identifying the amplification products of step (4) by hybridization to a nucleic acid sequence that is complementary to a portion of the amplification product, wherein nucleic acid sequence is covalently linked to a detectable moiety.

In some embodiments, amplification products are quantified by binding a second detectable moiety to said binding moiety.

In other embodiments, the binding moiety is biotin and said second binding moiety is avidin or streptavidin.

In further embodiments, the detectable moiety is a microsphere.

In other embodiments, steps (1)-(4) of the method are performed in a microtiter plate.

One aspect of the invention provides methods of treating or preventing a disorder characterized by mitochondrial dysfunction in a subject, the method comprising administering to the subject a therapeutically effective amount of a compound selected from mebendazole, cytochalasin E, deoxysappanone (deoxysappanone b 7,3′-dimethyl ether), nocodazole, paclitaxel podofilox, podophyllotoxin acetate or vinblastine, or a metabolite or analog thereof.

In some embodiments the mitochondrial dysfunction is characterized by reduced oxidative phosphorylation or increased generation of reactive oxygen species or both. In some embodiments, the disorder is diabetes, glucose intolerance, obesity, cardiac myopathy, premature aging, coronary atherosclerotic heart disease, diabetes mellitus, Alzheimer's Disease, Parkinson's Disease, Huntington's disease, dystonia, Leber's hereditary optic neuropathy (LHON), schizophrenia, myodegenerative disorders such as “mitochondrial encephalopathy, lactic acidosis, and stroke” (MELAS). and “myoclonic epilepsy ragged red fiber syndrome” (MERRF), NARP (Neuropathy; Ataxia; Retinitis Pigmentosa), MNGIE (Myopathy and external opthalmoplegia, neuropathy; gastro-intestinal encephalopathy), Keams-Sayre disease, Pearson's Syndrome, PEO (Progressive External Opthalmoplegia), congenital muscular dystrophy with mitochondrial structural abnormalities, Wolfram syndrome, Diabetes Insipidus, Diabetes Mellitus, Optic Atrophy Deafness, Leigh's Syndrome, fatal infantile myopathy with severe mitochondrial DNA (mtDNA) depletion, benign “later-onset” myopathy with moderate reduction in mtDNA, dystonia, medium chain acyl-CoA dehydrogenase deficiency, arthritis, mitochondrial diabetes and deafness (MIDD), or mitochondrial DNA depletion syndrome.

In exemplary embodiments the disorder is obesity and/or diabetes. In some embodiments, the disorder is glucose intolerance. In some embodiments, the disorder is premature aging. In some embodiments, the subject has elevated gluconeogenesis. In some embodiments, the subject is afflicted with cancer.

In some embodiments, methods for treating diabetes comprise administering a therapeutic dosage of paclitaxel or a metabolite or analog thereof.

In some embodiments, the disorder is a neurodegenerative disorder. In some embodiments, the neurodegenerative disorder is characterized by neuronal cell death. In some embodiments, the neurodegenerative disorder is Parkinson disease, amyotrophic lateral sclerosis (ALS), Alzheimer's disease, Huntington's disease, Freidreich's ataxia, Familial British Dementia, Finnish-type Familial Amyloidoses, Frontotemporal Dementia, Senile Systemic Amyloidosis, Familial Amyloid Polyneuropathy, Transmissible Spongiform Encephalopathie, Gertsmann-Strausseler-Scheinker Syndrome, Fatal Familial Insomnia, Huntington's Chorea, Kuru, Familial amyloid polyneuropathy, Creutzfeldt Jakob, Scrapie, and Bovine Spongiform Encephalopathy.

In some embodiments, the disorder is an mtDNA-associated disease. In some embodiments, the mt-DNA associated disease is MERRF, MELAS, LHON, MILASA, MILS, PEO or KSS.

In some embodiments, the disorder is a mitochondrial encephalomyopathy due to nuclear gene mutations. In some embodiments, the encephalomyopathy is Leigh syndrome French Canadian variety, mtDNA depletion syndromes, Barth syndrome and Wilson's disease.

One aspect of the invention also provides for compositions and combinations of compositions useful in treating or preventing a disorder characterized by mitochondrial dysfunction in a subject. In one embodiment, the composition comprises one or more of mebendazole, cytochalasin E, deoxysappanone, nocodazole, paclitaxel podofilox, podophyllotoxin acetate or vinblastine, or a metabolite or analog thereof.

In some embodiments, mebendazole or a metabolite or analog thereof is administered or formulated in a composition. In some embodiments, the subject is not afflicted with a worm infection.

In some embodiments, cytochalasin E or a metabolite or analog thereof is administered or formulated in a composition. In some embodiments of the methods, deoxysappanone or a metabolite or analog thereof is administered or formulated in a composition. In some embodiments, nocodazole or a metabolite or analog thereof is administered or formulated in a composition. In some embodiments, paclitaxel or a metabolite or analog thereof is administered or formulated in a composition. In some embodiments, podofilox or a metabolite or analog thereof is administered or formulated in a composition. In some embodiments, podophyllotoxin acetate or a metabolite or analog thereof is administered or formulated in a composition. In some embodiments, vinblastine or a metabolite or analog thereof is administered or formulated in a composition.

In some embodiments, one or more agents selected from sulfonylureas, non-sulfonylurea secretagogues, insulin, insulin analogs, glucagon-like peptides, exendin-4 polypeptides, beta 3 adrenoceptor agonists, PPAR agonists, dipeptidyl peptidase IV inhibitors, biguanides, alpha-glucosidase inhibitors, immunomodulators, statins and statin-containing combinations, angiotensin converting enzyme inhibitors, adenosine A1 receptor agonists, adenosine A2 receptor agonists, aldosterone antagonists, alpha 1 adrenoceptor antagonists, alpha 2 adrenoceptor agonists, alpha 2 adrenoceptor agonists, angiotensin receptor antagonists, antioxidants, ATPase inhibitors, atrial peptide agonists, beta adrenoceptor antagonists, calcium channel agonists, calcium channel antagonists, diuretics, dopamine D1 receptor agonists, endopeptidase inhibitors, endothelin receptor antagonists, guanylate cyclase stimulants, phosphodiesterase V inhibitors, protein kinase inhibitors, Cdc2 kinase inhibitors, renin inhibitors, thromboxane synthase inhibitors, vasopeptidase inhibitors, vasopressin I antagonists, vasopressin 2 antagonists, angiogenesis inhibitors, advanced glycation end product inhibitors, bile acid binding agents, bile acid transport inhibitors, bone formation stimulants, apolipoprotein A1 agonists, DNA topoisomerase inhibitors, cholesterol absorption inhibitors, cholesterol antagonists, cholesteryl ester transfer protein antagonists, cytokine synthesis inhibitors, DNA polymerase inhibitors, dopamine D2 receptor agonists, endothelin receptor antagonists, growth hormone antagonists, insulin sensitizers, lipase inhibitors, lipid peroxidation inhibitors, lipoprotein A antagonists, microsomal transport protein inhibitors, microsomal triglyceride transfer protein inhibitors, nitric oxide synthase inhibitors, oxidizing agents, phospholipase A2 inhibitors, radical formation agonists, platelet aggregation antagonists, prostaglandin synthase stimulants, reverse cholesterol transport activators, rho kinase inhibitors, selective estrogen receptor modulators, squalene epoxidase inhibitors, squalene synthase inhibitors, thromboxane A2 antagonists, amylin agonists, cannabinoid receptor antagonists, cholecystokinin A agonists, corticotropin-releasing factor agonists, dopamine uptake inhibitors, G protein-coupled receptor modulators, glutamate antagonists, glucagon-like peptide-1 agonists, insulin sensitizers, lipase inhibitors, melanin-concentrating hormone receptor antagonists, nerve growth factor agonists, neuropeptide Y agonists, neuropeptide Y antagonists, SNRIs, protein tyrosine phosphatase inhibitors, serotonin 2C receptor agonists, bezafibrate, diflunisal, or cinnamic acid may also be administered or formulated in a composition.

In some embodiments, sulfonylurea is selected from the group consisting of acetohexamide, chlorpropamide, tolazamide, tolbutamide, glimepiride, glipizide, and glyburide. In some embodiments, non-sulfonylurea secretagogue is nateglinide or repaglinide. In some embodiments, insulin analog is selected from the group consisting of insulin lispro, insulin aspart, insulin glarginine, NPH, lente insulin, ultralente insulin, humulin, and novolin. In some embodiments, PPAR.gamma. agonist is selected from the group consisting of balaglitazone, troglitazone, pioglitazone, ciglitazone, englitazone, rosiglitazone, darglitazone, englitazone, netoglitazone, KRP-297, JTT-501, NC-2100, NIP-223, MCC-555, L-764486, CS-011, G1262570, GW347845, and FK614. In some embodiments, biguanide is metformin or metformin/glyburide. In some embodiments, alpha-glucosidase inhibitor is acarbose or miglitol. In some embodiments, immunomodulator is a corticosteroid, cyclophosphamide, or NsIDI. In some embodiments, angiotensin converting enzyme (ACE) inhibitor is selected from the group consisting of benazepril, captopril, cilazapril, enalapril, enalaprilat, fosinopril, lisinopril, moexipril, perindopril, quinapril, ramipril, and trandolapril. In some embodiments, angiotensin II receptor blocker is selected from the group consisting of candesartan, eprosartan, irbesarten, losartin, telmisartan, and valsartan. In some embodiments, antioxidant is selected from the group consisting of nicotinamide, vitamin E, probucol, MDL29311, and U78518F. In some embodiments, exendin 4 is AC2993. In some embodiments, glucagon-like peptide is GLP-1.

### DETAILED DESCRIPTION OF THE INVENTION

### I. Overview

One aspect of the invention provides novel methods of treating disorders characterized by mitochondrial dysfunction. In one aspect, the disorders are characterized by reduced oxidative phosphorylation and/or increased production of reactive oxygen species (ROS). The disorders characterized by mitochondrial dysfunction may be treated by the administration of compounds disclosed herein. In some embodiments, the subject may be treated by the administration of mebendazole, cytochalasin E, deoxysappanone, nocodazole, paclitaxel podofilox, podophyllotoxin acetate or vinblastine, or a metabolite or analog thereof. In some embodiments, the disorders may be treated by the administration of a derivative of deoxysappone. These compounds may be administered in combination with other therapeutic agents. In addition, their pharmaceutically acceptable forms, including isomers such as diastereomers and enantiomers, salts, esters, solvates, and polymorphs thereof, as well as racemic mixtures and pure isomers of the compounds described herein, may be used in the treatments. In some embodiments, the methods of the invention comprise the administration of microtubule modulators which inhibit or promote tubulin polymerization.

One aspect of the invention provides methods of treating congenital mitochondrial diseases. These diseases are those related to hereditary mutations, deletions, or other defects in mitochondrial DNA or in nuclear genes regulating mitochondrial DNA integrity, or in nuclear genes encoding proteins that are critical for mitochondrial respiratory chain function. One aspect of the invention provides methods of treating acquired mitochondrial defects.

These comprise primarily 1) damage to mitochondrial DNA due to oxidative processes or aging; 2) mitochondrial dysfunction due to excessive intracellular and intramitochondrial calcium accumulation; 3) inhibition of respiratory chain complexes with endogenous or exogenous respiratory chain inhibitors; 4) acute or chronic oxygen deficiency; and 5) impaired nuclear-mitochondrial interactions, e.g. impaired shuttling of mitochondria in long axons due to microtubule defects.

In some embodiments, the mitochondrial disorders been treated by the compounds disclosed herein are characterized by excessive calcium accumulation. A fundamental mechanism of cell injury, especially in excitable tissues, involves excessive calcium entry into cells, as a result of either leakage through the plasma membrane or defects in intracellular calcium handling mechanisms. Mitochondria are major sites of calcium sequestration, and preferentially utilize energy from the respiratory chain for taking up calcium rather than for ATP synthesis, which results in a downward spiral of mitochondrial failure, since calcium uptake into mitochondria results in diminished capabilities for energy transduction.

In some embodiments, the mitochondrial disorders treatable by the compounds disclosed herein are characterized by excitotoxicity. Excessive stimulation of neurons with excitatory amino acids is a common mechanism of cell death or injury in the central nervous system. Activation of glutamate receptors, especially of the subtype designated NMDA receptors, results in mitochondrial dysfunction, in part through elevation of intracellular calcium during excitotoxic stimulation. Conversely, deficits in mitochondrial respiration and oxidative phosphorylation sensitize cells to excitotoxic stimuli, resulting in cell death or injury during exposure to levels of excitotoxic neurotransmitters or toxins that would be innocuous to normal cells.

In some embodiments, the mitochondrial disorders treatable by the compounds disclosed herein are characterized by nitric oxide exposure. Nitric oxide (1 micromolar) inhibits cytochrome oxidase (Complex IV) and thereby inhibits mitochondrial respiration. Moreover, prolonged exposure to NO irreversibly reduces Complex I activity. Physiological or pathophysiological concentrations of NO thereby inhibit pyrimidine biosynthesis. Nitric oxide is implicated in a variety of neurodegenerative disorders and is involved in mediation of excitotoxic and post-hypoxic damage to neurons.

In some embodiments, the mitochondrial disorders treatable by the compounds disclosed herein are characterized by hypoxia. Oxygen is the terminal electron acceptor in the respiratory chain. Oxygen deficiency impairs electron transport chain activity, resulting in diminished pyrimidine synthesis as well as diminished ATP synthesis via oxidative phosphorylation. Human cells proliferate and retain viability under virtually anaerobic conditions if provided with uridine and pyruvate (or a similarly effective agent for oxidizing NADH to optimize glycolytic ATP production).

In some embodiments, the mitochondrial disorders treatable by the compounds disclosed herein are characterized by nuclear-mitochondrial interactions. Transcription of mitochondrial DNA encoding respiratory chain components requires nuclear factors. In neuronal axons, mitochondria must shuttle back and forth to the nucleus in order to maintain respiratory chain activity. If axonal transport is impaired by hypoxia or by drugs like taxol that affect microtubule stability, mitochondria distant from the nucleus undergo loss of cytochrome oxidase activity.

The compounds and compositions of the invention are useful for treatment of a very broad spectrum of signs and symptoms in mitochondrial diseases with different underlying molecular pathologies, including those characterized by reduced oxidative phosphorylation and by generation of ROS. The broad applicability of the methods of the invention are unexpected. The set of compounds disclosed differ from other therapies of mitochondrial disease that have been attempted. For example, Coenzyme Q, B vitamins, carnitine, and lipoic acid, generally address very specific reactions and cofactors involved in mitochondrial function and which are therefore useful only in isolated cases. However, such metabolic interventions with antioxidants and cofactors of respiratory chain complexes are compatible with concurrent treatment with compounds and compositions of the invention and, in fact, are used to their best advantage in combination with compounds and compositions of the invention.

Treatment includes the application or administration of a therapeutic agent to a patient or application or administration of a therapeutic agent to an isolated tissue or cell line from a patient whom has a disease, a symptom of disease, or a predisposition toward a disease, with the purpose to cure, heal, alleviate, relieve, alter, remedy, ameliorate, improve or affect the disease, the symptoms of disease or the predisposition toward disease. The present invention also provides methods for screening compounds that enhance mitochondrial function, that are useful for treating disorders characterized by mitochondrial dysfunction, or that are contraindicated for patient use. As such, these methods can be used to prioritize large numbers of new compounds for further drug development. The adaptability of these in vitro methods for high-throughput analysis makes them an economical and cost-effective addition to a drug discovery program.

### II. Definitions

For convenience, certain terms employed in the specification, examples, and appended claims, are collected here. Unless defined otherwise, all technical and scientific terms used herein have the same meaning as commonly understood by one of ordinary skill in the art to which this invention belongs.

The articles “a” and “an” are used herein to refer to one or to more than one (i.e., to at least one) of the grammatical object of the article. By way of example, “an element” means one element or more than one element.

The term “including” is used herein to mean, and is used interchangeably with, the phrase “including but not limited” to.

The term “or” is used herein to mean, and is used interchangeably with, the term “and/or,” unless context clearly indicates otherwise.

The term “such as” is used herein to mean, and is used interchangeably, with the phrase “such as but not limited to”.

The term “nucleic acid” refers to polynucleotides such as deoxyribonucleic acid (DNA), and, where appropriate, ribonucleic acid (RNA). The term should also be understood to include, as equivalents, analogs of either RNA or DNA made from nucleotide analogs, and, as applicable to the embodiment being described, single (sense or antisense) and double-stranded polynucleotides.

The term “preventing” is art-recognized and when used in relation to a condition, such as a local recurrence (e.g., pain), a disease such as cancer, a syndrome complex such as heart failure or any other medical condition, is well understood in the art and includes administering prior to onset of the condition a composition that reduces the frequency of, reduces the severity of, or delays the onset of symptoms of a medical condition in a subject relative to a subject which does not receive the composition. Thus, prevention of cancer includes, for example, reducing the number of detectable cancerous growths in a population of patients receiving a prophylactic treatment relative to an untreated control population, and/or delaying the appearance of detectable cancerous growths in a treated population versus an untreated control population, e.g., by a statistically and/or clinically significant amount. Prevention of an infection includes, for example, reducing the number of diagnoses of the infection in a treated population versus an untreated control population, and/or delaying the onset of symptoms of the infection in a treated population versus an untreated control population.

The term “effective amount” as used herein is defined as an amount effective, at dosages and for periods of time necessary to achieve the desired result. The effective amount of a compound of the invention may vary according to factors such as the disease state, age, sex, and weight of the animal. Dosage regimens may be adjusted to provide the optimum therapeutic response. For example, several divided doses may be administered daily or the dose may be proportionally reduced as indicated by the exigencies of the therapeutic situation.

A “subject” as used herein refers to any vertebrate animal, preferably a primate or mammal, and more preferably a human. Examples of subjects include humans, non-human primates, rodents, guinea pigs, rabbits, sheep, pigs, goats, cows, horses, dogs, cats, birds, and fish.

By “treating, reducing, or preventing a metabolic disorder” it is meant ameliorating such a condition before or after it has occurred. As compared with an equivalent untreated control, such reduction or degree of prevention is at least 5%, 10%, 20%, 40%, 50%, 60%, 80%, 90%, 95%, or 100% as measured by any standard technique.

By “a metabolic disorder” is meant any pathological condition resulting from an alteration in a patient's metabolism. Such disorders include those resulting from an alteration in glucose homeostasis resulting, for example, in hyperglycemia. According to this invention, an alteration in glucose levels is typically an increase in glucose levels by at least 5%, 10%, 20%, 30%, 40%, 50%, 60%, 70%, 80%, 90%, or even 100% relative to such levels in a healthy individual. Metabolic disorders include obesity and diabetes (e.g., diabetes type I, diabetes type II, MODY, and gestational diabetes).

An “indicator of mitochondrial function” is any parameter that is indicative of mitochondrial function that can be measured by one skilled in the art. In certain embodiments, the indicator of mitochondrial function is a mitochondrial electron transport chain enzyme, a Krebs cycle enzyme, a mitochondrial matrix component, a mitochondrial membrane component or an ATP biosynthesis factor. In other embodiments, the indicator of mitochondrial function is mitochondrial number per cell or mitochondrial mass per cell. In other embodiments, the indicator of mitochondrial function is an ATP biosynthesis factor. In other embodiments, the indicator of mitochondrial function is the amount of ATP per mitochondrion, the amount of ATP per unit mitochondrial mass, the amount of ATP per unit protein or the amount of ATP per unit mitochondrial protein. In other embodiments, the indicator of mitochondrial function comprises free radical production. In other embodiments, the indicator of mitochondrial function comprises a cellular response to elevated intracellular calcium. In other embodiments, the indicator of mitochondrial function is the activity of a mitochondrial enzyme such as, by way of non-limiting example, citrate synthase, hexokinase II, cytochrome c oxidase, phosphofructokinase, glyceraldehyde phosphate dehydrogenase, glycogen phosphorylase, creatine kinase, NADH dehydrogenase, glycerol 3-phosphate dehydrogenase, triose phosphate dehydrogenase or malate dehydrogenase. In other embodiments, the indicator of mitochondrial function is the relative or absolute amount of mitochondrial DNA per cell in the patient.

“Improving, increasing, or enhancing mitochondrial function” or “altering mitochondrial function” may refer to (a) substantially (e.g., in a statistically significant manner, and preferably in a manner that promotes a statistically significant improvement of a clinical parameter such as prognosis, clinical score or outcome) restoring to a normal level at least one indicator of glucose responsiveness in cells having reduced glucose responsiveness and reduced mitochondrial mass and/or impaired mitochondrial function; or (b) substantially (e.g., in a statistically significant manner, and preferably in a manner that promotes a statistically significant improvement of a clinical parameter such as prognosis, clinical score or outcome) restoring to a normal level, or increasing to a level above and beyond normal levels, at least one indicator of mitochondrial function in cells having impaired mitochondrial function, or in cells having normal mitochondrial function, respectively. Improved or altered mitochondrial function may result from changes in extramitochondrial structures or events, as well as from mitochondrial structures or events, in direct interactions between mitochondrial and extramitochondrial genes and/or their gene products, or in structural or functional changes that occur as the result of interactions between intermediates that may be formed as the result of such interactions, including metabolites, catabolites, substrates, precursors, cofactors and the like.

“Impaired mitochondrial function” may include a full or partial decrease, inhibition, diminution, loss or other impairment in the level and/or rate of any respiratory, metabolic or other biochemical or biophysical activity in some or all cells of a biological source. As non-limiting examples, markedly impaired electron transport chain (ETC) activity may be related to impaired mitochondrial function, as may be generation of increased reactive oxygen species (ROS) or defective oxidative phosphorylation. As further examples, altered mitochondrial membrane potential, induction of apoptotic pathways and formation of atypical chemical and biochemical crosslinked species within a cell, whether by enzymatic or non-enzymatic mechanisms, may all be regarded as indicative of mitochondrial function. These and other non-limiting examples of impaired mitochondrial function are described in greater detail below.

A mitochondrial enzyme that may be an indicator of mitochondrial function

### III. Methods of Treatment

One aspect of the invention provides methods of treating, aiding in the treatment, preventing, or reducing the symptoms of a disorder characterized by mitochondrial dysfunction. Mitochondrial dysfunction may be diagnosed by a clinician. Symptoms of mitochondrial dysfunction may include idiopathic neuromuscular and/or multisystem disease or biochemical signs of energy depletion. Mitochondrial disorders are most commonly displayed as neuromuscular disorders, including developmental delay, seizure disorders, hypotonia, skeletal muscle weakness and cardiomyopathy. One method of identifying subjects having mitochondrial dysfunction is disclosed in U.S. Pat. No. 6,759,196. “Mitochondrial dysfunction” also refers to disorders to which deficits in mitochondrial respiratory chain activity contribute in the development of pathophysiology of such disorders in a mammal. This category includes 1) congenital genetic deficiencies in activity of one or more components of the mitochondrial respiratory chain; 2) acquired deficiencies in the activity of one or more components of the mitochondrial respiratory chain, wherein such deficiencies are caused by, inter alia, a) oxidative damage during aging; b) elevated intracellular calcium; c) exposure of affected cells to nitric oxide; d) hypoxia or ischemia; or e) microtubule-associated deficits in axonal transport of mitochondria.

One aspect of the invention provides methods of treating congenital mitochondrial cytopathies, the method comprising administering to the subject a therapeutically effective amount of one or more compounds described herein. In one embodiment, the method comprises administering to the subject a microtubule modulator. In one embodiment, the microtubule modulator is podofilox, vinblastine sulfate, mebendazole, pocodazole, podophyllotoxin, paclitaxela, albendazole, picropodophyllotoxin, griseofulvin, paclitaxel, coichicine, mebendazole, trifluralin, or griseofulvin

Congenital mitochondrial cytopathies include those characterized by mitochondrial DNA defects. A number of clinical syndromes have been linked to mutations or deletions in mitochondrial DNA. Mitochondrial DNA is inherited maternally with virtually all of the mitochondria in the body derived from those provided by the oocyte. If there is a mixture of defective and normal mitochondria in an oocyte, the distribution and segregation of mitochondria is a stochastic process. Thus, mitochondrial diseases are often multisystem disorders, and a particular point mutation in mitochondrial DNA, for example, can result in dissimilar sets of signs and symptoms in different patients. Conversely, mutations in two different genes in mitochondrial DNA can result in similar symptom complexes. Nonetheless, some consistent symptom patterns have emerged in conjunction with identified mitochondrial DNA defects, and these comprise the classic “mitochondrial diseases.” An important aspect of the subject invention is the recognition that the concept of mitochondrial disease and its treatment with compounds and compositions of the invention extends to many other disease conditions which are also disclosed herein.

Some of the major mitochondrial diseases associated with mutations or deletions of mitochondrial DNA include: MELAS (Mitochondrial Encephalomyopathy Lactic Acidemia and Stroke-like episodes), MERRF (Myoclonic Epilepsy with “Ragged Red” (muscle) Fibers), NARP (Neurogenic muscle weakness, Ataxia, and Retinitis Pigmentosa), LHON (Leber's Hereditary Optic Neuropathy), Leigh's Syndrome (Subacute Necrotizing Encephalomyopathy), PEO (Progressive External Opthalmoplegia), and Kearns-Sayres Syndrome (PEO, pigmentary retinopathy, ataxia, and heart-block). Other common symptoms of mitochondrial diseases that may be present alone or in conjunction with these syndromes include cardiomyopathy, muscle weakness and atrophy, developmental delays (involving motor, language, cognitive or executive function), ataxia, epilepsy, renal tubular acidosis, peripheral neuropathy, optic neuropathy, autonomic neuropathy, neurogenic bowel dysfunction, sensorineural deafness, neurogenic bladder dysfunction, dilating cardiomyopathy, migraine, hepatic failure, lactic acidemia, and diabetes mellitus.

In addition to the gene products and tRNA encoded by mitochondrial DNA, many proteins involved in or affecting mitochondrial respiration and oxidative phosphorylation are encoded by nuclear DNA. In fact, approximately 3000 proteins, or 20% of all proteins encoded by the nuclear genome, are physically incorporated into, or associated with, mitochondria and mitochondrial functions, although only about 100 are directly involved as structural components of the respiratory chain. Therefore, mitochondrial diseases involve not only gene products of mitochondrial DNA, but also nuclear encoded proteins affecting respiratory chain function.

Metabolic stressors, such as infection, can unmask mitochondrial defects that do not necessarily yield symptoms under normal conditions. Neuromuscular or neurological setbacks during infection are a hallmark of mitochondrial disease. Conversely, mitochondrial respiratory chain dysfunction can render cells vulnerable to stressors that would otherwise be innocuous.

One aspect of the invention provides methods of treating neuromuscular degenerative disorders, the method comprising administering to the subject a therapeutically effective amount of a compound described herein. In some embodiments, the compound is selected from mebendazole, cytochalasin E, deoxysappanone, nocodazole, paclitaxel podofilox, podophyllotoxin acetate or vinblastine, or a metabolite or analog thereof. In one embodiment, the method comprises administering to the subject a microtubule modulator.

In one embodiment, the neuromuscular degenerative disorder is Friedreich's Ataxia (FA). A gene defect underlying Friedreich's Ataxia (FA), the most common hereditary ataxia, was recently identified and is designated “frataxin”. In FA, after a period of normal development, deficits in coordination develop which progress to paralysis and death, typically between the ages of 30 and 40. The tissues affected most severely are the spinal cord, peripheral nerves, myocardium, and pancreas. Patients typically lose motor control and are confined to wheelchairs and are commonly afflicted with heart failure and diabetes. The genetic basis for FA involves GAA trinucleotide repeats in an intron region of the gene encoding frataxin. The presence of these repeats results in reduced transcription and expression of the gene. Frataxin is involved in regulation of mitochondrial iron content. When cellular frataxin content is subnormal, excess iron accumulates in mitochondria, promoting oxidative damage and consequent mitochondrial degeneration and dysfunction.

Compounds and compositions of the invention are useful for treating patients with disorders related to deficiencies or defects in frataxin, including Friedreich's Ataxia, myocardial dysfunction, diabetes mellitus and complications of diabetes like peripheral neuropathy. Conversely, diagnostic tests for presumed frataxin deficiencies involving PCR tests for GAA intron repeats are useful for identifying patients who will benefit from treatment with compounds and compositions of the invention.

In one embodiment, the neuromuscular degenerative disorder is muscular dystrophy (MD). MD refers to a family of diseases involving deterioration of neuromuscular structure and function, often resulting in atrophy of skeletal muscle and myocardial dysfunction. In the case of Duchenne muscular dystrophy, mutations or deficits in a specific protein, dystrophin, are implicated in its etiology. Mice with their dystrophin genes inactivated display some characteristics of muscular dystrophy, and have an approximately 50% deficit in mitochondrial respiratory chain activity. A final common pathway for neuromuscular degeneration in most cases is calcium-mediated impairment of mitochondrial function. Compounds and compositions of the invention are useful for reducing the rate of decline in muscular functional capacities and for improving muscular functional status in patients with muscular dystrophy.

In one embodiment, the neuromuscular degenerative disorder is multiple sclerosis (MS). MS (MS) is a neuromuscular disease characterized by focal inflammatory and autoimmune degeneration of cerebral white matter. Periodic exacerbations or attacks are significantly correlated with upper respiratory tract and other infections, both bacterial and viral, indicating that mitochondrial dysfunction plays a role in MS. Nitric oxide Depression of neuronal mitochondrial respiratory chain activity caused by Nitric Oxide (produced by astrocytes) is implicated as a molecular mechanism contributing to MS. Compounds and compositions of the invention are useful for treatment of patients with multiple sclerosis, both prophylactically and during episodes of disease exacerbation.

One aspect of the invention provides methods of treating seizure disorders, the method comprising administering to the subject a therapeutically effective amount of a compound described herein. In some embodiments, the compound is selected from mebendazole, cytochalasin E, deoxysappanone, nocodazole, paclitaxel podofilox, podophyllotoxin acetate or vinblastine, or a metabolite or analog thereof. In one embodiment, the method comprises administering to the subject a microtubule modulator. In one embodiment, the seizure disorder is epilepsy. The term “epilepsy” refers to any neurological condition that makes people susceptible to seizures. A seizure is a change in sensation, awareness, or behavior brought about by a brief electrical disturbance in the brain. Seizures vary from a momentary disruption of the senses, to short periods of unconsciousness or staring spells, to convulsions. Some people have just one type of seizure. Others have more than one type. Although they look different, all seizures are caused by the same thing: a sudden change in how the cells of the brain send electrical signals to each other. Epilepsy is often present in patients with mitochondrial cytopathies, involving a range of seizure severity and frequency, e.g. absence, tonic, atonic, myoclonic, and status epilepticus, occurring in isolated episodes or many times daily. In patients with seizures secondary to mitochondrial dysfunction, compounds and methods of the invention are useful for reducing frequency and severity of seizure activity.

The compounds of the invention may also be used to treat and prevent migraines. Metabolic studies on patients with recurrent migraine headaches indicate that deficits in mitochondrial activity are commonly associated with this disorder, manifesting as impaired oxidative phosphorylation and excess lactate production. Such deficits are not necessarily due to genetic defects in mitochondrial DNA. Migraine sufferers are hypersensitive to nitric oxide, an endogenous inhibitor of Cytochrome c Oxidase. In addition, patients with mitochondrial cytopathies, e.g. MELAS, often have recurrent migraines. In patients with recurrent migraine headaches, compounds, compositions, and methods of the invention are useful for prevention and treatment, especially in the case of headaches refractory to ergot compounds or serotonin receptor antagonists.

One aspect of the invention provides methods of treating mitochondrial-associated developmental delays, the method comprising administering to the subject a therapeutically effective amount of a compound described herein. In some embodiments, the compound is selected from mebendazole, cytochalasin E, deoxysappanone, nocodazole, paclitaxel podofilox, podophyllotoxin acetate or vinblastine, or a metabolite or analog thereof. In one embodiment, the method comprises administering to the subject a microtubule modulator.

Delays in neurological or neuropsychological development are often found in children with mitochondrial diseases. Development and remodeling of neural connections requires intensive biosynthetic activity, particularly involving synthesis of neuronal membranes and myelin, both of which require pyrimidine nucleotides as cofactors. Uridine nucleotides are involved in activation and transfer of sugars to glycolipids and glycoproteins. Cytidine nucleotides are derived from uridine nucleotides, and are crucial for synthesis of major membrane phospholipid constituents like phosphatidylcholine, which receives its choline moiety from cytidine diphosphocholine. In the case of mitochondrial dysfunction (due to either mitochondrial DNA defects or any of the acquired or conditional deficits like exicitoxic or nitric oxide-mediated mitochondrial dysfunction described above) or other conditions resulting in impaired pyrimidine synthesis, cell proliferation and axonal extension is impaired at crucial stages in development of neuronal interconnections and circuits, resulting in delayed or arrested development of neuropsychological functions like language, motor, social, executive function, and cognitive skills. In autism for example, magnetic resonance spectroscopy measurements of cerebral phosphate compounds indicates that there is global undersynthesis of membranes and membrane precursors indicated by reduced levels of uridine diphospho-sugars, and cytidine nucleotide derivatives involved in membrane synthesis (Minshew et al., Biological Psychiatry 33:762-773, 1993).

Disorders characterized by developmental delay include Rett's Syndrome, pervasive developmental delay (or PDD-NOS: “pervasive developmental delay—not otherwise specified” to distinguish it from specific subcategories like autism), autism, Asperger's Syndrome, and Attention Deficit/Hyperactivity Disorder (ADHD), which is becoming recognized as a delay or lag in development of neural circuitry underlying executive functions.

The compounds and compositions of the invention are useful for treating patients with neurodevelopmental delays involving motor, language, executive function, and cognitive skills. Current treatments for such conditions, e.g. ADHD, involve amphetamine-like stimulants that enhance neurotransmission in some affected underdeveloped circuits, but such agents, which may improve control of disruptive behaviors, do not improve cognitive function, as they do not address underlying deficits in the structure and interconnectedness of the implicated neural circuits. Compounds and compositions of the invention are also useful in the case of other delays or arrests of neurological and neuropsychological development in the nervous system and somatic development in non-neural tissues like muscle and endocrine glands.

One aspect of the invention provides methods of treating neurodegenerative disorders, the method comprising administering to the subject a therapeutically effective amount of a compound described herein. In some embodiments, the compound is selected from mebendazole, cytochalasin E, deoxysappanone, nocodazole, paclitaxel podofilox, podophyllotoxin acetate or vinblastine, or a metabolite or analog thereof. In one embodiment, the method comprises administering to the subject a microtubule modulator.

The two most significant severe neurodegenerative diseases associated with aging, Alzheimer's Disease (AD) and Parkinson's Disease (PD), both involve mitochondrial dysfunction in their pathogenesis. Complex I deficiencies in particular are frequently found not only in the nigrostriatal neurons that degenerate in Parkinson's disease, but also in peripheral tissues and cells like muscle and platelets of Parkinson's Disease patients.

In Alzheimer's Disease, mitochondrial respiratory chain activity is often depressed, especially Complex IV (Cytochrome c Oxidase). Moreover, mitochondrial respiratory function altogether is depressed as a consequence of aging, further amplifying the deleterious consequences of additional molecular lesions affecting respiratory chain function.

Other factors in addition to primary mitochondrial dysfunction underlie neurodegeneration in AD, PD, and related disorders. Excitotoxic stimulation and nitric oxide are implicated in both diseases, factors which both exacerbate mitochondrial respiratory chain deficits and whose deleterious actions are exaggerated on a background of respiratory chain dysfunction. Compounds and compositions of the invention are useful for attenuating progression of age-related neurodegenerative disease including AD and PD.

Huntington's Disease also involves mitochondrial dysfunction in affected brain regions, with cooperative interactions of excitotoxic stimulation and mitochondrial dysfunction contributing to neuronal degeneration.

In one embodiment, the neurodegenerative disease is Amyotrophic Lateral Sclerosis (ALS; Lou Gehrig's Disease) characterized by progressive degeneration of motor neurons, skeletal muscle atrophy, and inevitably leading to paralysis and death. ALS is caused by a mutation or deficiency in Copper-Zinc Superoxide Dismutase (SOD1), an antioxidant enzyme. Mitochondria both produce and are primary targets for reactive oxygen species. Inefficient transfer of electrons to oxygen in mitochondria is the most significant physiological source of free radicals in mammalian systems. Deficiencies in antioxidants or antioxidant enzymes can result in or exacerbate mitochondrial degeneration. Mice transgenic for mutated SOD1 develop symptoms and pathology similar to those in human ALS. The development of the disease in these animals has been shown to involve oxidative destruction of mitochondria followed by functional decline of motor neurons and onset of clinical symptoms (Kong and Xu, J. Neurosci. 18:3241-3250, 1998). Skeletal muscle from ALS patients has low mitochondrial Complex I activity (Wiedemann et al., J. Neurol. Sci 156:65-72, 1998). Compounds, compositions, and methods of the invention are useful for treatment of ALS, for reversing or slowing the progression of clinical symptoms.

One aspect of the invention provides methods of protecting against ischemia and hypoxia, the method comprising administering to the subject a therapeutically effective amount of a compound described herein. In some embodiments, the compound is selected from mebendazole, cytochalasin E, deoxysappanone, nocodazole, paclitaxel podofilox, podophyllotoxin acetate or vinblastine, or a metabolite or analog thereof. In one embodiment, the method comprises administering to the subject a microtubule modulator.

Oxygen deficiency results in both direct inhibition of mitochondrial respiratory chain activity by depriving cells of a terminal electron acceptor for Cytochrome c reoxidation at Complex IV, and indirectly, especially in the nervous system, via secondary post-anoxic excitotoxicity and nitric oxide formation. In conditions like cerebral anoxia, angina or sickle cell anemia crises, tissues are relatively hypoxic. In such cases, compounds of the invention provide protection of affected tissues from deleterious effects of hypoxia, attenuate secondary delayed cell death, and accelerate recovery from hypoxic tissue stress and injury.

Another condition where the compounds described here may be useful to protect against ischemia is renal tubular acidosis. Acidosis due to renal dysfunction is often observed in patients with mitochondrial disease, whether the underlying respiratory chain dysfunction is congenital or induced by ischemia or cytotoxic agents like cisplatin. Renal tubular acidosis often requires administration of exogenous sodium bicarbonate to maintain blood and tissue pH.

One aspect of the invention provides methods of treating diabetes, including Type II diabetes, the method comprising administering to the subject a therapeutically effective amount of a compound described herein. In some embodiments, the compound is selected from mebendazole, cytochalasin E, deoxysappanone, nocodazole, paclitaxel podofilox, podophyllotoxin acetate or vinblastine, or a metabolite or analog thereof. In one embodiment, the method comprises administering to the subject a microtubule modulator. Diabetes mellitus is a high prevalence illness characterized by high blood glucose levels. The chronic hyperglycemia (high glucose level) of diabetes is associated with long-term damage, dysfunction, and failure of various organs, especially the eyes, kidneys, nerves, heart, and blood vessels. The vast majority of cases of diabetes fall into two broad etiopathogenetic categories. The first category, type I or insulin-dependent diabetes mellitus (IDDM), results from an absolute deficiency of insulin due to autoimmunological destruction of the insulin-producing pancreatic β-cells. Another category, type 2 or non-insulin-dependent diabetes mellitus (NIDDM), which accounts for about 90% of all diabetes cases, is caused by a combination of resistance of insulin action and an inadequate compensatory insulin secretory response.

In one embodiment, the compound is administered in conjunction with other anti-diabetic treatments. Commonly used oral therapeutics for type 2 diabetes include thiazolidinediones (TZDs), sulfonylureas, metformin, and more recently, dipeptidyl peptidase IV (DPP-IV) inhibitors. Thiazolidinediones enhance insulin sensitivity by activating PPARγ receptors in adipose tissue and altering adipose metabolism and distribution (Spiegelman, 1998). Sulfonylureas promote insulin secretion by closing pancreatic cell potassium channels. Metformin decreases hepatocyte glucose production via an as yet unidentified mechanism of action. DPP-IV inhibitors are a new class of antidiabetic agent that prevents DPP-IV from degrading glucagon-like peptide-1 (GLP-1), a hormone that stimulates insulin secretion and reduces glucagon secretion from pancreas.

In one embodiment, administration of the compounds of the invention are useful for reducing glucose levels in a subject. By “reducing glucose levels” is meant reducing the level of glucose by at least 10%, 20%, 30%, 40%, 50%, 60%, 70%, 80%, 90%, 95%, or 100% relative to an untreated control. Desirably, glucose levels are reduced to normoglycemic levels, i.e., between 150 to 60 mg/dL, between 140 to 70 mg/dL, between 130 to 70 mg/dL, between 125 to 80 mg/dL, and preferably between 120 to 80 mg/dL. Such reduction in glucose levels may be obtained by increasing any one of the biological activities associated with the clearance of glucose from the blood. Accordingly, an agent having the ability to reduce glucose levels may increase insulin production, secretion, or action. Insulin action may be increased, for example, by increasing glucose uptake by peripheral tissues and/or by reducing hepatic glucose production.

Diagnosis of metabolic disorders, such as diabetes and glucose intolerance, may be performed using any standard method known in the art. Methods for diagnosing diabetes are described, for example, in U.S. Pat. No. 6,537,806, hereby incorporated by reference. Diabetes may be diagnosed and monitored using, for example, urine tests (urinalysis) that measure glucose and ketone levels (products of the breakdown of fat); tests that measure the levels of glucose in blood; glucose tolerance tests; and assays that detect molecular markers characteristic of a metabolic disorder in a biological sample (e.g., blood, serum, or urine) collected from the mammal (e.g., measurements of Hemoglobin Alc (HbAlc) levels in the case of diabetes).

Patients may be diagnosed as being at risk or as having diabetes if a random plasma glucose test (taken at any time of the day) indicates a value of 200 mg/dL or more, if a fasting plasma glucose test indicates a value of 126 mg/dL or more (after 8 hours), or if an oral glucose tolerance test (OGTT) indicates a plasma glucose value of 200 mg/dL or more in a blood sample taken two hours after a person has consumed a drink containing 75 grams of glucose dissolved in water. The OGTT measures plasma glucose at timed intervals over a 3-hour period. Desirably, the level of plasma glucose in a diabetic patient that has been treated according to the invention ranges between 160 to 60 mg/dL, between 150 to 70 mg/dL, between 140 to 70 mg/dL, between 135 to 80 mg/dL, and preferably between 120 to 80.

One skilled in the art will understand that patients treated by the methods of the invention may have been subjected to standard tests or may have been identified, without examination, as one at high risk due to the presence of one or more risk factors, such as family history, obesity, particular ethnicity (e.g., African Americans and Hispanic Americans), gestational diabetes or delivering a baby that weighs more than nine pounds, hypertension, having a pathological condition predisposing to obesity or diabetes, high blood levels of triglycerides, high blood levels of cholesterol, presence of molecular markers (e.g., presence of autoantibodies), and age (over 45 years of age). An individual is considered obese when their weight is 20% (25% in women) or more over the maximum weight desirable for their height. An adult who is more than 100 pounds overweight, is considered to be morbidly obese. Obesity is also defined as a body mass index (BMI) over 30 kg/m².

As indicated above, the methods of this invention may also be used prophylactically, i.e., in patients who are an increased risk of developing diabetes or a condition associated with diabetes. Risk factors include for example, family history of diabetes or obesity conditions, quality of nutrition, level of physical activity, presence of molecular markers of diabetes, age, race, or sex. Patients affected with other non-related disorders may also be predisposed to secondary diabetes.

One aspect of the invention provides methods of treating obesity, the method comprising administering to the subject a therapeutically effective amount of a compound described herein. In some embodiments, the compound is selected from mebendazole, cytochalasin E, deoxysappanone, nocodazole, paclitaxel, podofilox, podophyllotoxin acetate or vinblastine, or a metabolite or analog thereof. In one embodiment, the method comprises administering to the subject a microtubule modulator. Obesity is defined as a body mass index (BMI) of 30 kg/m² or more (National Institute of Health, Clinical Guidelines on the Identification, Evaluation, and Treatment of Overweight and Obesity in Adults (1998)). However, the invention is also intended to include a disease, disorder, or condition that is characterized by a body mass index (BMI) of 25 kg/m² or more, 26 kg/m² or more, 27 kg/m² or more, 28 kg/m² or more, 29 kg/m² or more, 29.5 kg/m² or more, or 29.9 kg/m² or more, all of which are typically referred to as overweight (National Institute of Health, Clinical Guidelines on the Identification, Evaluation, and Treatment of Overweight and Obesity in Adults (1998)).

One aspect of the invention provides methods of treating cardiovascular disease, the method comprising administering to the subject a therapeutically effective amount of a compound described herein. In some embodiments, the compound is selected from mebendazole, cytochalasin E, deoxysappanone, nocodazole, paclitaxel podofilox, podophyllotoxin acetate or vinblastine, or a metabolite or analog thereof. In one embodiment, the method comprises administering to the subject a microtubule modulator.

Cardiovascular disease includes hypertension, heart failure such as congestive heat failure or heart failure following myocardial infarction, arrhythmia, diastolic dysfunction such as left ventricular diastolic dysfunction, diastolic heart failure, or impaired diastolic filling, systolic dysfunction, ischemia such as myocardial ischemia, cardiomyopathy such as hypertrophic cardiomyopathy and dilated cardiomyopathy, sudden cardiac death, myocardial fibrosis, vascular fibrosis, impaired arterial compliance, myocardial necrotic lesions, vascular damage in the heart, vascular inflammation in the heart, myocardial infarction including both acute post-myocardial infarction and chronic post-myocardial infarction conditions, coronary angioplasty, left ventricular hypertrophy, decreased ejection fraction, coronary thrombosis, cardiac lesions, vascular wall hypertrophy in the heart, endothelial thickening, myocarditis, and coronary artery disease such as fibrinoid necrosis or coronary arteries.

In some embodiments, the heart disease is cardiomyopathy. Mitochondrial defects have been demonstrated to affect the heart, in particular leading to cardiomyopathy. (See Wallace D C, Am Heart J. 139(2 Pt 3):S70-85 (2000) and Fan, W. et al., Science 319:958-962 (2008)).

### IV. Compositions

### Cytoskeleton Modulators

In some embodiments of the methods described herein, the therapeutic compound that is administered to the subject is a cytoskeleton modulator. In some embodiments, the compound may modulate microfilaments, for example by promoting the polymerization or depolymerization of actin. In some embodiments, the compound may modulate microtubules, for example by promoting the polymerization or depolymerization of tubulin.

### Microfilament Modulators

In some embodiments of the methods described herein, the therapeutic compound administered to the subject is a microfilament modulator. Microfilaments are polymers of actin subunits.

In one embodiment of the methods described herein, the microfilament modulator administered to the subject is a cytochalasin derivative or a metabolite or analog thereof. “Cytochalasins” include fungal metabolites exhibiting an inhibitory effect on target cellular metabolism, including prevention of contraction or migration of vascular smooth muscle cells. Preferably, cytochalasins inhibit the polymerization of monomeric actin (G-actin) to polymeric form (F-actin). Cytochalasins typically are derived from phenylalanine (cytochalasins), tryptophan (chaetoglobosins), or leucine (aspochalasins), resulting in a benzyl, indol-3-yl methyl or isobutyl group, respectively, at position C-3 of a substituted perhydroisoindole-1-one moiety (Formula V or VI). The perhydroisoindole moiety in turn contains an 11-, 13- or 14-atom carbocyclic- or oxygen-containing ring linked to positions C-8 and C-9. All naturally occurring cytochalasins contain a methyl group at C-5; a methyl or methylene group at C-12; and a methyl group at C-14 or C-16. Exemplary molecules include cytochalasin A, cytochalasin B, cytochalasin C, cytochalasin D, cytochalasin E, cytochalasin F, cytochalasin G, cytochalasin H, cytochalasin J, cytochalasin K, cytochalasin L, cytochalasin M, cytochalasin N, cytochalasin O, cytochalasin P, cytochalasin Q, cytochalasin R, cytochalasin S, chaetoglobosin A, chaetoglobosin B, chaetoglobosin C, chaetoglobosin D, chaetoglobosin E, chaetoglobosin F, chaetoglobosin G, chaetoglobosin J, chaetoglobosin K, deoxaphomin, proxiphomin, protophomin, zygosporin D, zygosporin E, zygosporin F, zygosporin G, aspochalasin B, aspochalasin C, aspochalasin D and the like, as well as functional equivalents and derivatives thereof. In certain embodiments, the cytochalasin derivative is selected from cytochalasin A, cytochalasin B, cytochalasin C, cytochalasin D, cytochalasin E, cytochalasin F, cytochalasin H, cytochalasin J, cytochalasin K, cytochalasin Q, cytochalasin R, epoxycytochalasin H and epoxycytochalasin J.

In certain embodiments, the cytochalasin derivative administered to patients is cytochalasin E or a metabolite or analogue thereof. Cytochalasin E was first discovered as a toxic metabolite of Aspergillus clavatus (Buchi et al., J Am Chem Soc. 1973; 95(16):5423-5; Demain et al. Appl Environ Microbiol. 1976; 31(1):138-40). Cytochalasin E may be obtained by isolating and purifying from the culture medium of fungi capable of producing the compound in a manner similar to that described in J. Chem. Soc. Perkin Trans. 1, p. 541 (1982), and in Agric. Biol. Chem., Vol. 53, p. 1699 (1989). Cytochalasin E depolymerizes of actin filaments by binding to high affinity sites associated with F-actin. J Biol. Chem. 1980 Feb. 10; 255(3):835-8.

### Microtubule Modulators

In one embodiment of the methods described herein, the therapeutic compound that is administered to the subject is a microtubule modulator. Several compounds which affect microtubule assembly, disassembly, or function, for example through binding to or the stabilizing of microtubules, or through polymerization of tubulins to form microtubules, and the like, are known and include coumarin and dicoumarol (Jacobs, R. S. et al. U.S. Pub No. 2002/151560 A1), dictyostatin (Curran, D. P. et al., U52004186165 A1), eleutherobin (Lindel, T. et al., J. Am. Chem. Soc. 1997, 119(37), 8744-45), sarcodictyin Nicolaou, K. C., et al., WO9921862), epothilones (Goodin, S., et al., J. Gun Oncology, 2004, 22(10), 2015-25), FR182877 (Sato, B. et al., WO9632402), laulimalide and isolaulimalide (Mooberry, S. L., et al., Cancer Research, 1999, 59(3), 653-60), peloruside (Gaitanos, T. N., et al., Gancer Research, 2004, 64(15), 5063-67; and De Brabander, J. and Liao, X., US2004235939 A1), taccalonolides (Hemscheidt, T. K. and Mooberry, S. L., WO0071563), tubercidin (Mooberry, S. L., et al., Gancer Letters (Shaimon, Ireland), 1995, 96(2), 26 1-6), taxol and its analogs (Trojanowski, J. Q. and Lee, V. U.S. Pat. No. 5,580,898, 1996), discodermolide (Hung, D. T., et al., Chemistry and Biology, 1996, 3(4), 287-93; Haar, B., et al. Biochemistry, 1996, 35(1), 243-50; Kowaiski, R. L., et al., Molec. Pharm., 1997, 52, 6 13-22), and its analogs (Smith, et al., U.S. Pub No. 2002/0103387 A1 and PCT U502/24932), and the like, the reference each of which is hereby incorporated herein by reference, in its entirety. PCT Pub No. WO06/091728A2 discloses microtubule stabilizing compounds.

In one embodiment, the microtubule modulator is a microtubule stabilizing compound selected from coumarin, dicoumarol, dictyostatin, discodermolide, eleutherobin, sarcodictyin A or B, epothilone, FR182877, laulimalide, isolaulirnalide, peloruside, taccalonolide, or tubercidin, or any analog, or any combination, or both, thereof. In one embodiment, the anti-microtubule agent is selected from taxanes, discodermolide, colchicine, vinca alkaloids, and analogues or derivatives of any of these.

In one embodiment, the microtubule stabilizing agent effectively stabilizes microtubules at a physiologically compatible concentration. Microtubule stabilization typically is measured using a dose-response assay in which a sensitive assay system is contacted with a compound of interest over a range of concentrations at which no or minimal effect is observed, through higher concentrations at which partial effect is observed, to saturating concentrations at which a maximum effect is observed. Theoretically, such assays of the dose-response effect of stabilizer compounds can be expressed as a curve, expressing a degree of stabilization as a function of concentration. The curve also theoretically passes through a point at which the concentration is sufficient to stabilize microtubules to a level that is 50% that of the difference between minimal and maximal activity in the assay. This concentration is defined as the Inhibitory Concentration (50%) or IC₅₀ Comparisons between the efficacy of stabilizers often are provided with reference to comparative IC₅₀ concentrations, wherein a higher IC₅₀ indicates that the test compound is less potent, and a lower IC₅₀ indicates that the compound is more potent, than a reference compound. Similarly, the potency of stabilizer compounds can be related in terms of the Effective Concentration (50%) or EC₅₀, which is a measure of dose-response activity in a cell-based or animal-based model. EC₅₀ measurements are useful to relate properties of the compound that can influence its clinical utility, such as compound solubility, ability to penetrate cell membranes, partition coefficient, bioavailability, and the like. Two compounds can exhibit a divergence in comparative IC₅₀ and EC₅₀ values, i.e., one compound can be more potent in a biochemical assay and the second compound more potent in a cell-based assay simply due to different properties of the compounds.

In certain embodiments of the methods described herein, the microtubule modulator is represented by the structure of Formula (I):

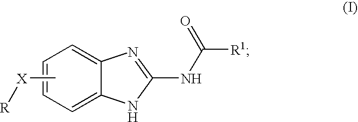

wherein R is selected from (C₁-C₄)alkyl, cycloalkyl having 3 to 6 carbon atoms, phenyl, halo-substituted phenyl in which halo in each occurrence is selected from Br, Cl, or F, (lower alkyl)-substituted phenyl, ((C₁-C₄)alkoxy)-substituted phenyl, and 2-thienyl; R¹ is selected from methyl and ethyl, X is selected from —S—, —C(O)—, —O—, —CH₂— and —S(O)— and the R—X— substituent is located at the 5(6)-position.

In one embodiment of the methods described herein, the therapeutic compound that is administered to the subject is methyl[5-benzoyl-benzimidazol-2-carbamate] (mebendazole) or a metabolite or analog thereof. In one embodiment, mebendazole is administered to a subject not afflicted with, or at risk of being afflicted with, a worm infection, including hookworm infection, a roundworm infection, a pinworm infection or a whipworm infection. In one embodiment, mebendazole is administered to a subject not afflicted with diabetes. Commercially-available compositions that may be used in the methods of the invention include Ovex®, Vermox®, Antiox® or Pripsen®. In one embodiment, the mebendazole is administered as oral tablets, such as 100 mg chewable tablets. U.S. Patent Pub No. 2005/0038096 discloses mebendazole containing compositions that may be used in the methods described herein. Mebendazole is also described in Campell, W. C. et al. J. Parasitol. 61:844-852 (1975); Heath, D. D. et al. Parasitology 70:273-285 (1975). Mebendazole is a tubulin inhibitor.

In one embodiment of the methods described herein, the therapeutic compound that is administered to the subject is methyl[5-(2-thienylcarbonyl)-1H-benzimidazol-2-yl]carbamate (nocodazole) or a metabolite or analog thereof. Nocodazole is a microtubule inhibitor that prevents the addition of tubulin molecules to microtubules, thereby disturbing the equilibrium and leading to microtubule depolymerization and destruction of the spindle. Nocodazole may be obtained from Sigma-Aldrich.

In one embodiment of the methods described herein, the therapeutic compound that is administered to the subject is selected from albendazole, fenbendazole, oxfendazole, oxibendazole, methiazole, and parbendazole.

In certain aspects of the methods described herein, the therapeutic compound administered to the subject is represented by the structure of Formula (II):

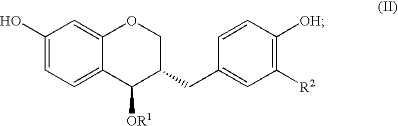

wherein R¹ is selected from H or methyl and R² is selected from H or hydroxy. In certain embodiments, the therapeutic compound administered to the subject is selected from a compound represented by a structure of Formulas (III)-(VI):

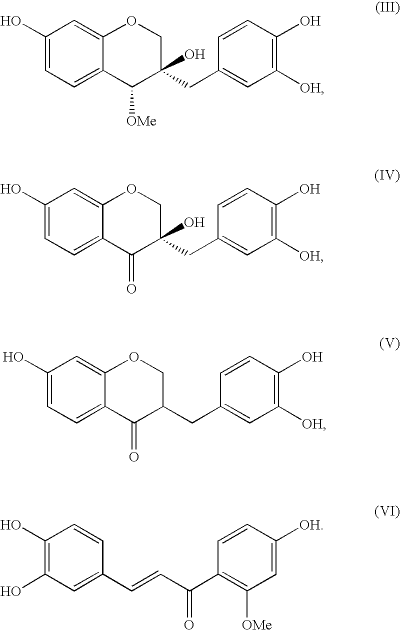

In certain embodiments, the therapeutic compound administered to the subject is the compound of Formula (V), deoxysappanone B, or a metabolite, analog or derivative thereof. In one embodiment, deoxysappanone (B) is selected from deoxysappanone (B) 7,3′-dimethyl ether; deoxysappanone (B) 7,3′-trimethyl ether; sappanone (A) trimethyl ether; 3-deshydroxysappanol trimethyl ether; sappanone (A) 7-methyl ether; tetrahydrosappanone (A) trimethyl ether; sappanone (A) dimethyl ether; and deoxysappanone (B) 7,3′-dimethyl ether acetate. In one embodiment, the therapeutic compound administered to the subject is deoxysappanone (B) 7,3′-dimethyl ether, sappanone (A) trimethyl ether, or 3-deshydroxysappanol trimethyl ether. In one embodiment, deoxysappanone B, or a metabolite, analog or derivative thereof is administered to a subject not afflicted with diabetes.

In certain embodiments, the therapeutic compound administered to the subject is represented by the structure of Formula (VII):

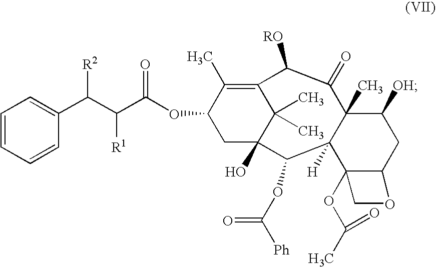

wherein, R is nitrogen or acetyl and one of R¹ and R² is hydroxy and the other is selected from t-butylcarbonylamino or benzoylamino. In one embodiment of the methods described herein, the therapeutic compound that is administered to the subject is paclitaxel (Taxol) or a metabolite or analog thereof. Paclitaxel is an anti-microtubule agent extracted from the needles and bark of the Pacific yew tree. U.S. Patent Pub No. 2006/0281933 provides a method of synthesizing paclitaxel. Paclitaxel may be formulated as a concentrated solution containing paclitaxel, 6 mg per milliliter of Cremophor EL (polyoxyethylated castor oil) and dehydrated alcohol (50% v/v) and must be further diluted before administration (Goldspiel, “Taxol pharmaceutical issues: preparation, administration, stability, and compatibility with other medications,”]Ann. Pharmacotherapy, 28:S23-26, 1994.).

In one embodiment, a soluble paclitaxel form of paclitaxel is administered that includes solubilizing moieties such as succinate, sulfonic acid, amino acids; and phosphate derivatives at the 2′-hydroxyl group or at the 7-hydroxyl position (Deutsch et al., “Synthesis of congeners and prodrugs. Water-soluble prodrugs of paclitaxel with potent antitumor activity,” J. Med. Chem., 32:788-792, 1989; Mathew et al., “Synthesis and evaluation of some water-soluble prodrugs and derivatives of taxol with antitumor activity,” J. Med. Chem., 35:145-151, 1992; Nicolaou, Riemer, Kerr, Rideout, Wrasidio, “Design, synthesis and biological activity of protaxols,” Nature, 364:464-466, 1993; Vyas et al., “Phosphatase-activated prodrugs of paclitaxel,” In: Taxane Anticancer Agents: Basic Science and Current Status, Georg, Chen, Ojima, Vyas. eds., American Chemical Society, Washington, D.C., 124-137, 1995; Rose, et al., “Preclinical antitumor activity of water-soluble paclitaxel derivatives,” Cancer Chemother. Pharmacol., 39:486-492, 1997).

Additional derivatives and analogs of paclitaxel, as well as formulations, that may be used in methods of the invention are described in U.S. Patent Pub Nos: 2006/0135404, 2006/0052312, 2004/0198638, 2003/0176320, 2003/0166507, 2003/0147807, 2003/0134793, 2003/0130341, 2003/0130178, 2003/0130170, 2003/0124055, 2003/0114518, 2003/0114397, 2003/0114363, 2003/0113335, 2005/0191323, 2005/0016926, 2002/0103254. Paclitaxel is commercially available as Onxol® and Taxol®.

In one embodiment of the methods described herein, the therapeutic compound that is administered to the subject is podofilox or a metabolite or analog thereof. Podofilox, also called podophyllotoxin, is a purer and more stable form of podophyllin in which only the biologically active portion of the compound is present. Like podophyllin, it is used to treat genital warts. It has several advantages of podophyllin, however. Podofilox is commercially available as Condylox®, and it is manufactured by Oclassen Pharmaceuticals.

In one embodiment of the methods described herein, the therapeutic compound that is administered to the subject is podophyllotoxin acetate or a metabolite or analog thereof. Podophyllotoxin is a well-known lignan which has been isolated from plant extracts, particularly from so-called Podophyllum resins obtained by solvent extraction of various parts—notably the roots and rhizomes—of plants of the genus Podophyllum, e.g. the North American species Podophyllum peltatum and the Indian species Podophyllum emodi. Podophyllotoxin has been reported to occur in a variety of polymorphic forms having different melting points, and in the form of various solvates [see, e.g., A. W. Schrecker et al., J. Org. Chem. 21 (1956) 288]. Schrecker et al. recognized at least four crystalline modifications of podophyllotoxin: (A), with water (m.p. 161° C.-162° C.); (B), unsolvated (m.p. 183° C.-184° C.); (C), with water and benzene of crystallization (m.p. 114° C.-118° C. “foaming”); and (D), unsolvated (m.p. 188° C.-189° C.). U.S. Patent Pub. 2006/0293254 describes a podophyllotoxin that may be used in the treatments described herein. U.S. Pat. No. 5,315,016 discloses a process for preparing pure podophyllotoxin. U.S. Pat. No. 4,680,399: discloses a process for the isolation and purification of podophyllotoxin. PCT Pub. No. WO01/52826A2 discloses podophyllotoxin compositions. U.S. Pat. No. 5,336,605 discloses the production of podophyllotoxins using podophyllum.

In certain embodiments, the therapeutic compound administered to the subject is represented by the structure of Formula (VIII):

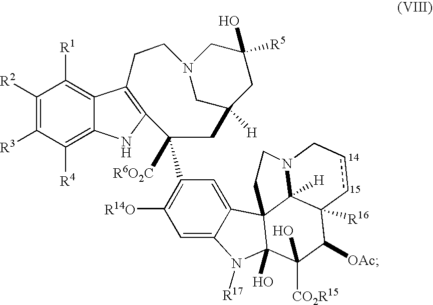

wherein R¹, R², R³ and R⁴ are independently selected from H, lower alkyl group, lower alkoxy group, halogen, lower perfluoroalkyl group, lower alkylthio group, hydroxy group, amino group, mono- or di-alkyl or acylamino group, lower alkyl or arylsulfonyloxy group, R⁵ is H, or a lower alkyl group or a substituted or non-substituted aryl group, R⁶ is an alkyl group of carbon number 4 or less, R¹⁴, R¹⁵ and R¹⁶ are an alkyl group of carbon number 4 or less, R¹⁷ is H or an alkyl group of carbon number 4 or less, and in between carbon 14 and carbon 15 is an unsaturated double bond or saturated bond.

In one embodiment of the methods described herein, the therapeutic compound that is administered to the subject is vinblastine or a metabolite or analog thereof. Vinblastine inhibits palmitoylation of tubulin and is therefore a microtubule inhibitor. PCT Pub. No. WO88/03135 discloses a method of isolating vinblastine. U.S. Pat. No. 4,749,787 discloses a process for isolating vinblastine from the plant catharanthis roseus. U.S. Pub No. 2006/0293357 discloses intermediates for synthesis of vinblastine, a process for preparation of the intermediates and a process for synthesis of vinblastines. U.S. Pat. No. 5,397,784 discloses stable parenteral compositions of vinblastine or vincristine. U.S. Pat. No. 4,870,162 discloses conjugates of vinblastine, a process for their preparation and their use in therapy. U.S. Pat. No. 4,910,138 discloses the use of an organ culture of Catharanthus roseus to produce vincristine and vinblastine. U.S. Pat. No. 4,639,456 discloses vinblastin-23-oyl amino acid derivatives. U.S. Pat. No. 4,362,664 discloses vinblastine oxazolidinedione disulfides and related compounds. U.S. Pat. No. 4,305,875 discloses a process for the synthesis of vinblastine and leurosidine. U.S. Pat. No. 4,188,394 discloses ophthalmic compositions of vinblastine. In certain embodiments, the therapeutic compound that is administered to the subject is vincristine.

### V. Screening Methods

One aspect of the invention provides for methods for identifying compounds that enhance mitochondrial function. Mitochondrial function can be evaluated based on a number of criteria. These include mitochondrial respiratory activity, which may decrease when mitochondrial function is impaired, and mitochondrial membrane potential, which may decrease when mitochondrial function is impaired.

The methods disclosed herein provide assaying for the effect of one or more compounds on OXPHOS gene expression and mitochondrial function and correlating the effect determined from those assays on mitochondrial function. An increase in OXPHOS gene expression and an increase in mitochondrial function are indicative of compounds that enhance mitochondrial function.

In some embodiments, the mitochondrial function is assayed by measuring reactive oxygen species (ROS), and an increase in OXPHOS gene expression and a decrease in ROS is indicative of a compound that enhances mitochondrial function. In some embodiments, the method further comprises assaying for the effect of one or more compounds on cell viability. In some embodiments, the method further comprises assaying for the effect of one or more compounds on dehydrogenase activity, mitochondrial membrane potential, cellular ATP, and cytochrome c protein.

Examples 1 and 2 provide exemplary embodiments of methods for identifying compounds than enhance mitochondrial function.

One aspect of the invention provides for methods for identifying compounds useful in treating a disorder characterized by mitochondrial dysfunction in a subject. The methods comprise assaying for the effect of one or more compounds on OXPHOS gene expression and mitochondrial function and correlating the effect determined from those assays on mitochondrial function. An increase in OXPHOS gene expression and an increase of mitochondrial function are indicative of compounds useful in treating a disorder.

In some embodiments, the mitochondrial function is assayed by measuring reactive oxygen species (ROS) and an increase in OXPHOS gene expression and a decrease in ROS is indicative of a compound that enhances mitochondrial function. In some embodiments, the method further comprises assaying for the effect of one or more compounds on cell viability. In some embodiments, the method further comprises assaying for the effect of one or more compounds on dehydrogenase activity, mitochondrial membrane potential, cellular ATP, and cytochrome c protein.

Examples 1 and 2 provide exemplary embodiments of methods for identifying compounds that enhance mitochondrial function.

In some embodiments of the screening methods, the disorder characterized by mitochondrial dysfunction is MELAS (Mitochondrial Encephalomyopathy Lactic Acidemia and Stroke-like episodes), MERRF (Myoclonic Epilepsy with “Ragged Red” (muscle) Fibers), NARP (Neurogenic muscle weakness, Ataxia, and Retinitis Pigmentosa), LHON (Leber's Hereditary Optic Neuropathy), Leigh's Syndrome (Subacute Necrotizing Encephalomyopathy), PEO (Progressive External Opthalmoplegia), and Keams-Sayres Syndrome (PEO, pigmentary retinopathy, ataxia, and heart-block). In some embodiments, the disorder characterized by mitochondrial dysfunction is diabetes. In some embodiments, the disorder characterized by mitochondrial dysfunction is type II diabetes mellitus. In some embodiments, the disorder characterized by mitochondrial dysfunction is cardiomyopathy. In some embodiments, the disorder characterized by mitochondrial dysfunction is Parkinson's disease. In some embodiments, the disorder characterized by mitochondrial dysfunction is Huntington's disease. In some embodiments, the disorder characterized by mitochondrial dysfunction is premature aging.

One aspect of the invention provides for methods for determining compounds that are contraindicated in a subject. A compound is contraindicated when administration increases the risk in a subject of suffering negative consequences. A contraindication may be absolute, i.e. the compound should never be administered to a subject, or relative, i.e., the risks involved must be balanced against each other. It is within the purview of one skilled in the art to examine the risk of administering compounds identified in this screen and determine on an individual patient basis whether the risk is acceptable or not.

The methods comprise assaying for the effect of one or more compounds on dehydrogenase activity and cell viability and correlating the effect determined from those assays to a contraindication of a compound. A decrease in cellular dehydrogenase activity absent a decrease in cell viability indicates that the compound is contraindicated. In some embodiments, the effect of one or more compounds on cellular ATP is also determined and a decrease in ATP levels indicates that the compound is contraindicated.

In some embodiments, the method further comprises assaying for the effect of one or more compounds on mitochondrial membrane potential, OXPHOS gene expression, reactive oxygen species and cytochrome c protein. A decrease in membrane potential, an decrease in OXPHOS gene expression, an increase in ROS, and a decrease in cytochrome c levels are all indicators that suggest the compound is contraindicated.

In some embodiments, the subject is afflicted with a disorder characterized by mitochondrial dysfunction.

One aspect of the invention provides for determining two or more compounds that are contraindicated for joint administration to a subject. As demonstrated in Example 4, propranolol has an additive effect on statin-induced decrease in ATP levels. The screening methods described herein, provide for determining compounds that when jointly administered impair mitochondrial function.

The methods comprise assaying for the effect of two or more compounds on dehydrogenase activity and cell viability and correlating the effect determined from those assays to a contraindication of a combination of compounds. A decrease in cellular dehydrogenase activity absent a decrease in cell viability in two or more compounds indicates that administration of the two or more compounds are contraindicated. In some embodiments, the effect of two or more compounds on cellular ATP is also determined and a decrease in ATP levels indicates that the administration of the combination of compounds is contraindicated.

In some embodiments, the method further comprises assaying for the effect of two or more compounds on mitochondrial membrane potential, OXPHOS gene expression, reactive oxygen species and cytochrome c protein. A decrease in membrane potential, an decrease in OXPHOS gene expression, an increase in ROS, and a decrease in cytochrome c levels are all indicators that suggest the combination of compounds is contraindicated.

In some embodiments, the subject is afflicted with a disorder characterized by mitochondrial dysfunction.

In some embodiments of the methods, the subject is afflicted with MELAS (Mitochondrial Encephalomyopathy Lactic Acidemia and Stroke-like episodes), MERRF (Myoclonic Epilepsy with “Ragged Red” (muscle) Fibers), NARP (Neurogenic muscle weakness, Ataxia, and Retinitis Pigmentosa), LHON (Leber's Hereditary Optic Neuropathy), Leigh's Syndrome (Subacute Necrotizing Encephalomyopathy), PEO (Progressive External Opthalmoplegia), and Keams-Sayres Syndrome (PEO, pigmentary retinopathy, ataxia, and heart-block). In some embodiments, the subject is afflicted with diabetes. In some embodiments, the subject is afflicted with type II diabetes mellitus. In some embodiments, the subject is afflicted with cardiomyopathy. In some embodiments, the subject is afflicted with Parkinson's disease. In some embodiments, the subject is afflicted with Huntington's disease. In some embodiments, the subject is afflicted with premature aging.

The methods described herein utilize a variety of cell-based assays. Such a cell may be a primary cell in culture or it may be a cell line. In some embodiments, the cells are murine myotubes. In some embodiments, the cells are seeded in multiwell plates and allowed to reach log phase growth.

Once the cell cultures are thus established, various concentrations of the compound being tested are added to the media and the cells are allowed to grow exposed to the various concentrations for 6, 12, 24, 36, 48 or more hours. It should be noted that testing the specific compounds for longer or shorter periods of time is contemplated to be within the scope of the invention. Increased culture times may sometimes reveal additional cytotoxicity information at the cost of slowing down the screening process.

Furthermore, the cells may be exposed to the test compound at any given phase in the growth cycle. For example, in some embodiments, it may be desirable to contact the cells with the compound at the same time as a new cell culture is initiated. Alternatively, it may be desirable to add the compound when the cells have reached confluent growth or arc in log growth phase. Determining the particular growth phase cells are in is achieved through methods well known to those of skill in the art.

In an exemplary set of assays, the test compound concentration range comprises dosing solutions which yield final growth media concentration of 0.05 micromolar, 0.1 micromolar, 1.0 micromolar, 5.0 micromolar, 10.0 micromolar, 20.0 micromolar, 50.0 micromolar, 100 micromolar, and 300 micromolar of the compound in culture media. As mentioned, these are exemplary ranges, and it is envisioned that any given assay will be run in at least two different concentrations, and the concentration dosing may comprise, for example, 4, 5, 6, 7, 8, 9, 10, 11, 12, 13, 14, 15 or more concentrations of the compound being tested. Such concentrations may yield, for example, a media concentration of 0.05 micromolar, 0.1 micromolar, 0.5 micromolar, 1.0 micromolar, 2.0 micromolar, 3.0 micromolar, 4.0 micromolar, 5.0 micromolar, 10.0 micromolar, 15.0 micromolar, 20.0 micromolar, 25.0 micromolar, 30.0 micromolar, 35.0 micromolar, 40.0 micromolar, 45.0 micromolar, 50.0 micromolar, 55.0 micromolar, 60.0 micromolar, 65.0 micromolar, 70.0 micromolar, 75.0 micromolar, 80.0 micromolar, 85.0 micromolar, 90.0 micromolar, 95.0 micromolar, 80.0 micromolar, 110.0 micromolar, 120.0 micromolar, 130.0 micromolar, 140.0 micromolar, 150.0 micromolar, 160.0 micromolar, 170.0 micromolar, 180.0 micromolar, 190.0 micromolar, 200.0 micromolar, 210.0 micromolar, 220.0 micromolar, 230.0 micromolar, 240.0 micromolar, 250.0 micromolar, 260.0 micromolar, 270.0 micromolar, 280.0 micromolar, 290.0 micromolar, and 300 micromolar in culture media. It will be apparent that a cost-benefit balancing exists in which the testing of more concentrations over the desired range provides additional information, but at additional cost, due to the increased number of cell cultures, assay reagents, and time required. In one embodiment, ten different concentrations over the range of 0 micromolar to 300 micromolar are screened.

Assays that measure mitochondrial physiology are indicators of mitochondrial function. Compounds that alter mitochondrial function may either up- or down regulating oxidative respiration. It should be noted that the screening methods provided herein allow for compounds to be screened using a number of different assays. This permits a more accurate prediction of the compound's in vivo effects. It should be noted that for some compounds the assays may provide conflicting results. It is within the purview of one skilled in the art to analyze the results of the assays in their entirety and reach a conclusion as to the compound's overall effects.

One assay provided by the invention measures changes in OXPHOS gene expression. The assay to measure changes in OXPHOS gene expression may measure the changes of any number of OXPHOS genes, as described in Mootha, V. K., et al., Nat. Genet. 34: 267-273 (2003). In some embodiments, the assay measures the changes in expression of the following genes (a) Mt-Atp6 (Entrez GeneID numbers 17705 or 4508), (b) Mt-Atp8 (Entrez GeneID numbers 17706 or 4509), (c) Mt-Co1 (Entrez GeneID numbers 17708 or 4512), (d) Mt-Co2 (Entrez GeneID numbers 17709 or 4513), (e) Mt-Co3 (Entrez GeneID numbers 17710 or 4514), (f) Mt-Cytb (Entrez GeneID number 17711 or 4519), (g) Mt-Nd1 (Entrez GeneID numbers 17716 or 4535), (h) Mt-Nd2 (Entrez GeneID numbers 17717 or 4536), (i) Mt-Nd3 (Entrez GeneID numbers 17718 or 4537), (j) Mt-Nd4 (Entrez GeneID numbers 17719 or 4538), (k) Mt-Nd41 (Entrez GeneID numbers 17720 or 4539), (l) Mt-Nd5 (Entrez GeneID numbers 17721 or 4540), (m) Mt-Nd6 (Entrez GeneID numbers 17722 or 4541), (n) Atp5a1 (Entrez GeneID numbers 11946 or 498), (o) Atp5c1 (Entrez GeneID numbers 11949 or 509), (p) Atp5o (Entrez GeneID numbers 28080 or 539), (q) Cox5b (Entrez GeneID numbers 12859 or 1329), (r) Cox7a2 (Entrez GeneID numbers 12866 or 1347), (s) Cyc1 (Entrez GeneID numbers 66445 or 1537), (t) Hspc051 (Entrez GeneID number 66152 or 29796), (u) Ndufa5 (Entrez GeneID numbers 68202 or 4698), (v) Ndufb5 (Entrez GeneID numbers 66046 or 4711), (w) Sdhd (Entrez GeneID numbers 66925 or 6392), (x) Uqcrb (Entrez GeneID numbers 67530 or 7381), and (y) Uqcrc1 (Entrez GeneID numbers 22273 or 7384).

In some embodiments, expression of OXPHOS genes is measured using a system designed to assess the presence and/or the quantity of any given transcript. In some embodiments, the system can be used for thousands of samples. In some embodiments, primer pairs are used to amplify a target sequence on an OXPHOS gene. The target sequence may be the entire gene or any appropriate region thereof. In some embodiments, the primer pairs may comprise nucleic acids that bind under stringent conditions to the target sequences. In other embodiments, the primer pairs may be linked to tag sequences. In some embodiments, tag sequences may be any nucleic acid sequence that does not hybridize to the target sequence. In certain embodiments, tag sequences may be selected from a set of over 100 sequences that are known in the art. In some embodiments, the primer pairs may also be linked to an additional nucleic acid sequence. In some embodiments, the primer pairs will be linked to tag sequences and tag sequences will be further linked to additional nucleic acid sequences. In some embodiments, the additional nucleic acid sequence will not hybridize to either the target sequence or the tag sequences. In some embodiments, the tag sequence will be linked to the 5′ end of the primer in the primer pair. In some embodiments, the additional nucleic acid sequence will be linked to the 5′ end of the tag sequence. In certain embodiments, the additional nucleic acid sequences will comprise binding sites for universal primers. In some embodiments, universal primers are sequences that may be used to amplify simultaneously all desired targets in a reaction mix. In some embodiments, universal primers may be selected from nucleic acid sequences that are found in humans, non-human mammals, plants, fungi, bacteria, or viruses. In some embodiments, universal primers are derived from the DNA sequence of a bacteriophage, such as the promoter for the RNA polymerases T7, SP6, or T3. Any nucleic acid sequences in all embodiments may also be further modified by addition or removal of groups such as phosphates, methyl groups, or labels known in the art.

In some embodiments, the tag sequences comprise any one of SEQ ID NOs 71-105, listed in Table 9. In some embodiments, the additional nucleic acid sequence comprises the binding site for a universal primer, such as, but not limited to, T3 or T7. In some embodiments, the universal primers comprise either one of SEQ ID NOs 106-107, listed in Table 9. The primer sequences set forth herein may be combined with any one of the tag sequences provided herein or known in the art. For example, SEQ ID 108 is a primer sequence comprising the tag of SEQ ID NO: 76 linked to the universal primer of SEQ ID NO: 106 and further linked to the target specific primer of SEQ ID NO: 1. Other exemplary combinations are listed in Table 10 (SEQ ID NO: 108-176), and represent a subset of possible combinations.

In some embodiments, target sequences are identified in a pool of transcripts isolated from a sample. In some embodiments, the transcripts may be captured by binding to immobilized poly-dT. In other embodiments, a plurality of primers that hybridizes under stringent conditions to the target sequences is added. Copies of the target sequences are produced from the primers, using reverse transcriptase and ligase. In some embodiments, each primer further comprises a tag sequence linked to the primer, such that the resultant copy of the target sequence contains at least one copy of a tag sequence. In some embodiments, the tag sequence is linked to the 5′ end of the primer. In other embodiments, each primer is linked to a tag sequence plus an additional nucleic acid sequence, such as a site complementary to a universal primer, and the resultant copy of the target sequence contain at least one copy of a tag sequence and is flanked by sites for universal primers. In some embodiments, a pair of universal primers can then be used to amplify the copies of the target sequences. In some embodiments, one of the universal primers is phosphorylated, and the other is linked to a binding moiety. Thus, a final amplification product is produced in these embodiments, wherein the amplification product contains the following nucleic acid sequences: (1) at least one portion of the target sequence, (2) a tag sequence, (3) universal primer sites, and (4) a binding moeity. In some embodiments, detection of the final amplification product requires the binding of the tag sequence to a complementary nucleic acid sequence that has been conjugated to a detectable moiety. In some embodiments, the detectable moiety is a microsphere. In further embodiments, the microsphere is colored, such that a reaction mix containing more than one colored microsphere can be distinguished from others by flow cytometry.

In other embodiments, the levels of OXPHOS gene expression are quantified by measuring the quantity of the amplification products. In some embodiments, the binding moieties on the amplification products are measured. Examples of binding moieties include but are not limited to proteins, epitope tags, small molecules, aptamers, nucleic acid sequences, proteins and antibodies to any of the preceding. In some embodiments, the binding moieties are biotin, avidin, or streptavidin. In other embodiments, the quantity of the binding moiety is determined indirectly, for example, by quantifying a second binding moiety that attaches to the binding moiety. In some embodiments, the second binding moiety is conjugated to a label such as a fluorescent, enzymatic, chemilumiscent, or calorimetric label, which can then be detected by a laser scanner, or CCD camera, or X-ray film, depending on the label, or other appropriate means of detecting a particular label, and quantified. Examples of labels include but are not limited to molecules such as fluorescein, Eosin Y, Rhodamine, Rose Bengal, Sulforhodamine, acridine yellow, proflavin, DDAO, cresyl violet, nile blue, oxazine, Cy2, Cy3, Cy5, Cy7, Alexa Fluors, coumarin, chlorophyll; fluorescent proteins such as DsRed, GFP and variations of GFP such as EGFP, YFP, CFP, RFP; phycocyanin, phycoerythrin; molecules such as luciferase, digoxygenin, alkaline phosphatase, and HRP.

In some embodiments, the expression level of genes is weighted to determine a Composite Z-score. Each gene is weighted by its ability to distinguish DMSO control wells from PGC-1α-treated wells. The signal-to-noise ratio of each gene is calculated using a PGC-α-treated positive control and DMSO negative control. The expression value of each gene per well is multiplied by this signal-to-noise ratio. The weighted scores are summed over nuclear-encoded or mitochondrial-encoded OXPHOS genes to derive one score each for expression within each genome. The Composite Z-score is exemplified in the tables as GE-HTS. In some embodiments, an increase in OXPHOS gene expression is a GE-HTS value greater than 0.5, 1.0, 1.5, 1.8, 2.0, 2.2, 2.4, 2.6, 2.8, 3.0, 3.2, 3.4, or 3.6. In some embodiments, a decrease in OXPHOS gene expression is a GE-HTS value less than 1.0, 0.5, 0.3, 0.0, −0.1, −0.2, −0.5, −0.8, −1.0, −1.2, −1.5, −2.0, −2.5, or −3.0.

One assay useful in the methods described herein is an assay to measure reactive oxygen species. Biologically reactive oxygen species include, but are not limited to: i) superoxide (O₂); ii) peroxides (ROOH) such as, but not limited to, hydrogen peroxide (H₂O₂) or hypochlorite (OCl⁻); and iii) hydroxide radical (OH). Biologically reactive nitrogen species include, but are not limited to, nitric oxide (NO), nitrogen dioxide (NO₂), or peroxynitrate (ONOO⁻). In the candidate screening assays H₂O₂/free radical measurement may be measured using kits (kit available from Molecular Probes-Invitrogen) or reporter molecule undergoing conformational change in the presence of free radical/H₂O₂ (quantitative fluorescent output). A Composite Z-score is determined as described above (see also on the World Wide Web at chembank.broad.harvard.edu/details.htm?tag=Help#screeningData). A Composite Z-score is exemplified in the tables as ROS. In some embodiments, an increase in ROS is a score greater than 0.5, 1.0, 1.5, 1.8, 2.0, 2.2, 2.4, 2.6, 2.8, 3.0, 3.2, 3.4, or 3.6. In some embodiments, a decrease in ROS is a score less than 1.0, 0.5, 0.3, 0.0, −0.1, −0.2, −0.5, −0.8, −1.0, −1.2, −1.5, −2.0, −2.5, or −3.0.

Another example of an assay that measures mitochondrial physiology is an assay for mitochondrial membrane potential. Typically, mitochondrial membrane potential may be determined according to methods with which those skilled in the art will be readily familiar, including but not limited to detection and/or measurement of detectable compounds such as fluorescent indicators, optical probes and/or sensitive pH and ion-selective electrodes (See, e.g., Ernster et al., 1981 J. Cell Biol. 91:227s and references cited; see also Haugland, 1996 Handbook of Fluorescent Probes and Research Chemicals, Sixth Ed., Molecular Probes, Eugene, Oreg., pp. 266-274 and 589-594.). For example, by way of illustration and not limitation, the fluorescent probes 2-,4-dimethylaminostyryl-N-methylpyridinium (DASPMI) and tetramethylrhodamine esters (e.g., tetramethylrhodamine methyl ester, TMRM; tetramethylrhodamine ethyl ester, TMRE) or related compounds (see, e.g., Haugland, 1996, supra) may be quantified following accumulation in mitochondria, a process that is dependent on, and proportional to, mitochondrial membrane potential (see, e.g., Murphy et al., 1998 in Mitochondria & Free Radicals in Neurodegenerative Diseases, Beal, Howell and Bodis-Wollner, Eds., Wiley-Liss, New York, pp. 159-186 and references cited therein; and Molecular Probes On-line Handbook of Fluorescent Probes and Research Chemicals, on the world wide web at probes.com/handbook/toc.html). Other fluorescent detectable compounds that may be used include but are not limited to rhodamine 123, rhodamine B hexyl ester, DiOC.sub.6(3), JC-1 [5,5′,6,6′-Tetrachloro-1,1′,3,3′-Tetraethylbezimidazolcarbocyanine Iodide] (see Cossarizza, et al., 1993 Biochem. Biophys. Res. Comm. 197:40; Reers et al., 1995 Meth. Enzymol. 260:406), rhod-2 (see U.S. Pat. No. 5,049,673; all of the preceding compounds are available from Molecular Probes, Eugene, Oreg.) and rhodamine 800 (Lambda Physik, GmbH, Gottingen, Germany; see Sakanoue et al., 1997 J. Biochem. 121:29). Methods for monitoring mitochondrial membrane potential are also disclosed in U.S. patent application Ser. No. 09/161,172. A Composite Z-score is determined as described above (see also on the World wide Web at chembank.broad.harvard.edu/details.htm?tag=Help#screeningData). A Composite Z-score for mitochondrial membrane potential measured using the JC-1 assay is exemplified in the tables as ΔΨₘ. In some embodiments, an increase in mitochondrial membrane potential is a score greater than 0.5, 1.0, 1.5, 1.8, 2.0, 2.2, 2.4, 2.6, 2.8, 3.0, 3.2, 3.4, or 3.6. In some embodiments, a decrease in membrane potential is a score less than 1.0, 0.5, 0.3, 0.0, −0.1, −0.2, −0.5, −0.8, −1.0, −1.2, −1.5, −2.0, −2.5, or −3.0.

Another example of an assay that measures mitochondrial physiology is an assay for cellular ATP levels. ATP can provide information on the energy status of the cell and provides a marker to assess early changes in mitochondrial function. Assays that allow a determination of ADP/ATP energy balance are well known in the art (Kangas et al., Med Biol, 62, 338-343, 1984). A Composite Z-score is determined as described above (see also on the World Wide Web at chembank.broad.harvard.edu/details.htm?tag=Help#screeningData). A Composite Z-score for the cellular ATP levels is exemplified in the tables as ATP. In some embodiments, an increase in cellular ATP levels is a score greater than 0.5, 1.0, 1.5, 1.8, 2.0, 2.2, 2.4, 2.6, 2.8, 3.0, 3.2, 3.4, or 3.6. In some embodiments, a decrease in cellular ATP levels is a score less than 1.0, 0.5, 0.3, 0.0, −0.1, −0.2, −0.5, −0.8, −1.0, −1.2, −1.5, −2.0, −2.5, or −3.0.

Mitochondria physiology and function can also be evaluated by measuring mitochondrial dehydrogenase activity. In one embodiment, mitochondrial dehydrogenase activity is measured using the MTT assay. Mitochondria catalyze the reduction of 3-(4,5-dimethylthiazol-2-yl)-2,5-diphenyltetrazolium bromide (MTT) to a blue or purple formazan compound. The relatively insoluble formazan blue is extracted into isopropanol and the absorbance of the extract measured. A high absorbance value indicates viable cells and functional mitochondria. Conversely, a decrease in the intensity of color suggests either a loss of cells, or direct toxic effects on the mitochondria. The MTT assay is well known to those of skill in the art and has been described in for example, the MTT mitochondrial dye assay is described in Mosmann, J. Immunol. Methods 65, 55-63, 1983 and in Denizot et al., J. Immunol. Methods. 89, 271-277, 1986. A Composite Z-score is determined as described above (see also World Wide Web at chembank.broad.harvard.edu/details.htm?tag=Help#screeningData). A Composite Z-score for the dehydrogenase assay is exemplified in the tables as MTT. In some embodiments, an increase in dehydrogenase activity is a score greater than 0.5, 1.0, 1.5, 1.8, 2.0, 2.2, 2.4, 2.6, 2.8, 3.0, 3.2, 3.4, or 3.6. In some embodiments, a decrease in dehydrogenase activity is a score less than 1.0, 0.5, 0.3, 0.0, −0.1, −0.2, −0.5, −0.8, −1.0, −1.2, −1.5, −2.0, −2.5, or −3.0.

A further exemplary assay measures cytochrome c protein levels. A Composite Z-score is determined as described above (see also on the World Wide Web at chembank.broad.harvard.edu/details.htm?tag=Help#screeningData). A Composite Z-score for the cytochrome c assay is exemplified in the tables as cyt c. In some embodiments, an increase in cytochrome c levels is a score greater than 0.5, 1.0, 1.5, 1.8, 2.0, 2.2, 2.4, 2.6, 2.8, 3.0, 3.2, 3.4, or 3.6. In some embodiments, a decrease in cytochrome c levels is a score less than 1.0, 0.5, 0.3, 0.0, −0.1, −0.2, −0.5, −0.8, −1.0, −1.2, −1.5, −2.0, −2.5, or −3.0.

An additional assay useful in the screening methods described herein is a cell viability assay. This assay distinguishing between compounds that are generally toxic to a cell versus those with a more specific effect on mitochondrial function. Cell viability assays are widely known to one skilled in the art. In one embodiment, the assay utilizes calcein dye. A Composite Z-score is determined as described above (see also on the World Wide Web at chembank.broad.harvard.edu/details.htm?tag=Help#screeningData). A Composite Z-score for the cell viability assay is exemplified in the tables as Viability. In some embodiments a lack of a decrease on cell viability is a score greater than −0.5, 0.0, 0.5, 1.0, 1.5, 1.8, 2.0, 2.2, 2.4, 2.6, 2.8, 3.0

High throughput assays for screening numerous compounds are specifically contemplated. In certain embodiments, the high throughput screens may be automated. In high throughput screening assays, groups of compounds are exposed to a biological target. These groups may be assembled from collections of compounds previously individually prepared and since stored in a compound bank, the assembly being random or guided by the use of similarity programs from which similar structures are formed. The assays provided herein are optimized to be used in a high throughput format. In some embodiments the assays are performed in a multi-well plate. In some embodiments, the assays are performed in a 384-well plate.

In certain aspects of the present invention, all the necessary components for conducting the assays may be packaged into a kit. Specifically, the present invention provides a kit for use in an assay, the kit comprising a packaged set of reagents for conducting two or more assays selected from the group consisting of a OXPHOS gene expression assay, cell viability assay, mitochondrail membrane potential assay, cellular ATP assay, dehydrogenase assay, ROS assay, and cytochrome C detection assay. In addition to the reagents, the kit may also include instructions packaged with the reagents for performing one or more variations of the assays of the invention using the reagents. The instructions may be fixed in any tangible medium, such as printed paper, or a computer-readable magnetic or optical medium, or instructions to reference a remote computer data source such as a worldwide web page accessible via the internet.

In some embodiments, a kit is provided for determining OXPHOS gene expression, comprising a set of primer pairs, each pair amplifying an OXPHOS gene selected from a group consisting of the following: (a) Mt-Atp6 (Entrez GeneID numbers 17705 or 4508), (b) Mt-Atp8 (Entrez GeneID numbers 17706 or 4509), (c) Mt-Co1 (Entrez GeneID numbers 17708 or 4512), (d) Mt-Co2 (Entrez GeneID numbers 17709 or 4513), (e) Mt-Co3 (Entrez GeneID numbers 17710 or 4514), (f) Mt-Cytb (Entrez GeneID number 17711 or 4519), (g) Mt-Nd1 (Entrez GeneID numbers 17716 or 4535), (h) Mt-Nd2 (Entrez GeneID numbers 17717 or 4536), (i) Mt-Nd3 (Entrez GeneID numbers 17718 or 4537), (j) Mt-Nd4 (Entrez GeneID numbers 17719 or 4538), (k) Mt-Nd41 (Entrez GeneID numbers 17720 or 4539), (l) Mt-Nd5 (Entrez GeneID numbers 17721 or 4540), (m) Mt-Nd6 (Entrez GeneID numbers 17722 or 4541), (n) Atp5a1 (Entrez GeneID numbers 11946 or 498), (o) Atp5c1 (Entrez GeneID numbers 11949 or 509), (p) Atp5o (Entrez GeneID numbers 28080 or 539), (q) Cox5b (Entrez GeneID numbers 12859 or 1329), (r) Cox7a2 (Entrez GeneID numbers 12866 or 1347), (s) Cyc1 (Entrez GeneID numbers 66445 or 1537), (t) Hspc051 (Entrez GeneID number 66152 or 29796), (u) Ndufa5 (Entrez GeneID numbers 68202 or 4698), (v) Ndufb5 (Entrez GeneID numbers 66046 or 4711), (w) Sdhd (Entrez GeneID numbers 66925 or 6392), (x) Uqcrb (Entrez GeneID numbers 67530 or 7381), and (y) Uqcrc1 (Entrez GeneID numbers 22273 or 7384).

In some embodiments, the kit comprises primer pairs that hybridize under stringent conditions to a target sequence, which may be the entire gene or any appropriate region thereof.

In some embodiments, the kit comprises a first primer comprising the nucleotide sequence of SEQ ID NO: 1 and a second primer comprising the nucleotide sequence of SEQ ID NO: 2; the second primer pair comprises a first primer comprising the nucleotide sequence of SEQ ID NO: 3 and a second primer comprising the nucleotide sequence of SEQ ID NO: 4; the third primer pair comprises a first primer comprising the nucleotide sequence of SEQ ID NO: 5 and a second primer comprising the nucleotide sequence of SEQ ID NO: 6; the fourth primer pair comprises a first primer comprising the nucleotide sequence of SEQ ID NO: 7 and a second primer comprising the nucleotide sequence of SEQ ID NO: 8; the fifth primer pair comprises a first primer comprising the nucleotide sequence of SEQ ID NO: 9 and a second primer comprising the nucleotide sequence of SEQ ID NO: 10, the sixth primer pair comprises a first primer comprising the nucleotide sequence of SEQ ID NO: 11 and a second primer comprising the nucleotide sequence of SEQ ID NO: 12, the seventh primer pair comprises a first primer comprising the nucleotide sequence of SEQ ID NO: 13 and a second primer comprising the nucleotide sequence of SEQ ID NO: 14, the eighth primer pair comprises a first primer comprising the nucleotide sequence of SEQ ID NO: 15 and a second primer comprising the nucleotide sequence of SEQ ID NO: 16, the ninth primer pair comprises a first primer comprising the nucleotide sequence of SEQ ID NO: 17 and a second primer comprising the nucleotide sequence of SEQ ID NO: 18, the tenth primer pair comprises a first primer comprising the nucleotide sequence of SEQ ID NO: 19 and a second primer comprising the nucleotide sequence of SEQ ID NO: 20, the eleventh primer pair comprises a first primer comprising the nucleotide sequence of SEQ ID NO: 21 and a second primer comprising the nucleotide sequence of SEQ ID NO: 22, the twelfth primer pair comprises a first primer comprising the nucleotide sequence of SEQ ID NO: 23 and a second primer comprising the nucleotide sequence of SEQ ID NO: 24, the thirteenth primer pair comprises a first primer comprising the nucleotide sequence of SEQ ID NO: 25 and a second primer comprising the nucleotide sequence of SEQ ID NO: 26, the fourteenth primer pair comprises a first primer comprising the nucleotide sequence of SEQ ID NO: 27 and a second primer comprising the nucleotide sequence of SEQ ID NO: 28, the fifteenth primer pair comprises a first primer comprising the nucleotide sequence of SEQ ID NO: 29 and a second primer comprising the nucleotide sequence of SEQ ID NO: 30, the sixteenth primer pair comprises a first primer comprising the nucleotide sequence of SEQ ID NO: 31 and a second primer comprising the nucleotide sequence of SEQ ID NO: 32, the seventeenth primer pair comprises a first primer comprising the nucleotide sequence of SEQ ID NO: 33 and a second primer comprising the nucleotide sequence of SEQ ID NO: 34, the eighteenth primer pair comprises a first primer comprising the nucleotide sequence of SEQ ID NO: 35 and a second primer comprising the nucleotide sequence of SEQ ID NO: 36, the nineteenth primer pair comprises a first primer comprising the nucleotide sequence of SEQ ID NO: 37 and a second primer comprising the nucleotide sequence of SEQ ID NO: 38, the twentieth primer pair comprises a first primer comprising the nucleotide sequence of SEQ ID NO: 39 and a second primer comprising the nucleotide sequence of SEQ ID NO: 40, the twenty-first primer pair comprises a first primer comprising the nucleotide sequence of SEQ ID NO: 41 and a second primer comprising the nucleotide sequence of SEQ ID NO: 42, the twenty-second primer pair comprises a first primer comprising the nucleotide sequence of SEQ ID NO: 43 and a second primer comprising the nucleotide sequence of SEQ ID NO: 44, the twenty-third primer pair comprises a first primer comprising the nucleotide sequence of SEQ ID NO: 45 and a second primer comprising the nucleotide sequence of SEQ ID NO: 46, the twenty-fourth primer pair comprises a first primer comprising the nucleotide sequence of SEQ ID NO: 47 and a second primer comprising the nucleotide sequence of SEQ ID NO: 48, the twenty-fifth primer pair comprises a first primer comprising the nucleotide sequence of SEQ ID NO: 49 and a second primer comprising the nucleotide sequence of SEQ ID NO: 50.

In some embodiments, the kit further comprises at least one primer pair that amplifies a gene showing little or no upregulation by PGC-1a. In some embodiments, at least one primer pair amplifies a gene selected from (a) Actb (Entrez GeneID 11461), (b) Aamp (Entrez GeneID 227290), (c) Cenpb (Entrez GeneID 12616), (d) Eefla1 (Entrez GeneID 13627), (e) Jund (Entrez GeneID 16478), (f) Lsp1 (Entrez GeneID 16985), (g) Rps2 (Entrez GeneID 16898), and (h) Rps27a (Entrez GeneID 78294). In some embodiments, the first primer pair comprises a first primer comprising the nucleotide sequence of SEQ ID NO: 51 and a second primer comprising the nucleotide sequence of SEQ ID NO: 52; the second primer pair comprises a first primer comprising the nucleotide sequence of SEQ ID NO: 53 and a second primer comprising the nucleotide sequence of SEQ ID NO: 54; the third primer pair comprises a first primer comprising the nucleotide sequence of SEQ ID NO: 55 and a second primer comprising the nucleotide sequence of SEQ ID NO: 56; the fourth primer pair comprises a first primer comprising the nucleotide sequence of SEQ ID NO: 57 and a second primer comprising the nucleotide sequence of SEQ ID NO: 58; the fifth primer pair comprises a first primer comprising the nucleotide sequence of SEQ ID NO: 59 and a second primer comprising the nucleotide sequence of SEQ ID NO: 60, the sixth primer pair comprises a first primer comprising the nucleotide sequence of SEQ ID NO: 61 and a second primer comprising the nucleotide sequence of SEQ ID NO: 62, the seventh primer pair comprises a first primer comprising the nucleotide sequence of SEQ ID NO: 63 and a second primer comprising the nucleotide sequence of SEQ ID NO: 64, the eighth primer pair comprises a first primer comprising the nucleotide sequence of SEQ ID NO: 65 and a second primer 66.

In some embodiments, the kit further comprises at least one primer pair that amplifies a gene that is down-regulated by PGC-1α. In some embodiments, the primer pair amplifies a gene selected from (a) Cyb5r3 (Entrez Gene ID 109754), and (b) Fhl1 (Entrez Gene ID 14199). In some embodiments, the first primer pair comprises a first primer comprising the nucleotide sequence of SEQ ID NO: 67 and a second primer comprising the nucleotide sequence of SEQ ID NO: 68; the second primer pair comprises a first primer comprising the nucleotide sequence of SEQ ID NO: 69 and a second primer comprising the nucleotide sequence of SEQ ID NO: 70. In some embodiments, the kit further comprises reagents for amplifying DNA, wherein the reagents include a DNA polymerase.

### VI. Formulations

Any of the compounds employed according to the present invention may be contained in any appropriate amount in any suitable carrier substance, and is generally present in an amount of 1-95% by weight of the total weight of the composition. The composition may be provided in a dosage form that is suitable for the oral, parenteral (e.g., intravenously, intramuscularly), rectal, cutaneous, nasal, vaginal, inhalant, skin (patch), or ocular administration route. Thus, the composition may be in the form of, e.g., tablets, capsules, pills, powders, granulates, suspensions, emulsions, solutions, gels including hydrogels, pastes, ointments, creams, plasters, drenches, osmotic delivery devices, suppositories, enemas, injectables, implants, sprays, or aerosols. The pharmaceutical compositions may be formulated according to conventional pharmaceutical practice (see, e.g., Remington: The Science and Practice of Pharmacy, 20th edition, 2000, ed. A. R. Gennaro, Lippincott Williams & Wilkins, Philadelphia, and Encyclopedia of Pharmaceutical Technology, eds. J. Swarbrick and J. C. Boylan, 1988-1999, Marcel Dekker, New York).

If more than one agent is employed, each agent may be formulated in a variety of ways that are known in the art. In one embodiment, the agents are formulated together for the simultaneous or near simultaneous administration of the agents. Such co-formulated compositions can include the two agents formulated together in the same pill, capsule, liquid, etc. It is to be understood that, when referring to the formulation of such combinations, the formulation technology employed is also useful for the formulation of the individual agents of the combination, as well as other combinations of the invention. By using different formulation strategies for different agents, the pharmacokinetic profiles for each agent can be suitably matched.

The individually or separately formulated agents can be packaged together as a kit. Non-limiting examples include kits that contain, e.g., two pills, a pill and a powder, a suppository and a liquid in a vial, two topical creams, etc. The kit can include optional components that aid in the administration of the unit dose to patients, such as vials for reconstituting powder forms, syringes for injection, customized IV delivery systems, inhalers, etc. Additionally, the unit dose kit can contain instructions for preparation and administration of the compositions. The kit may be manufactured as a single use unit dose for one patient, multiple uses for a particular patient (at a constant dose or in which the individual compounds may vary in potency as therapy progresses); or the kit may contain multiple doses suitable for administration to multiple patients (“bulk packaging”). The kit components may be assembled in cartons, blister packs, bottles, tubes, and the like.

In one embodiment, the therapeutic agent is formulated with a pharmaceutically acceptable carrier. Examples of materials which can serve as pharmaceutically acceptable carriers include sugars, such as lactose, glucose and sucrose; starches, such as corn starch and potato starch; cellulose, and its derivatives, such as sodium carboxymethyl cellulose, ethyl cellulose and cellulose acetate; powdered tragacanth; malt; gelatin; talc; excipients, such as cocoa butter and suppository waxes; oils, such as peanut oil, cottonseed oil, safflower oil, sesame oil, olive oil, corn oil and soybean oil; glycols, such as propylene glycol; polyols, such as glycerin, sorbitol, mannitol and polyethylene glycol; esters, such as ethyl oleate and ethyl laurate; agar; buffering agents, such as magnesium hydroxide and aluminum hydroxide; alginic acid; pyrogen-free water; isotonic saline; Ringer's solution; ethyl alcohol; pH buffered solutions; polyesters, polycarbonates and/or polyanhydrides; and other non-toxic compatible substances employed in pharmaceutical formulations. Wetting agents, emulsifiers and lubricants, such as sodium lauryl sulfate and magnesium stearate, as well as coloring agents, release agents, coating agents, sweetening, flavoring and perfuming agents, preservatives and other antioxidants can also be present in the compositions.

The compounds may be formulated with pharmaceutically acceptable salts. The term “pharmaceutically acceptable salt” refers to salts which retain the biological effectiveness and properties of the compounds of this invention and which are not biologically or otherwise undesirable. In many cases, the compounds of this invention are capable of forming acid and/or base salts by virtue of the presence of amino and/or carboxyl groups or groups similar thereto. Pharmaceutically acceptable base addition salts can be prepared from inorganic and organic bases. Salts derived from inorganic bases, include by way of example only, sodium, potassium, lithium, ammonium, calcium and magnesium salts. Salts derived from organic bases include, but are not limited to, salts of primary, secondary and tertiary amines, such as alkyl amines, dialkyl amines, trialkyl amines, substituted alkyl amines, di(substituted alkyl) amines, tri(substituted alkyl) amines, alkenyl amines, dialkenyl amines, trialkenyl amines, substituted alkenyl amines, di(substituted alkenyl) amines, tri(substituted alkenyl) amines, cycloalkyl amines, di(cycloalkyl) amines, tri(cycloalkyl) amines, substituted cycloalkyl amines, disubstituted cycloalkyl amine, trisubstituted cycloalkyl amines, cycloalkenyl amines, di(cycloalkenyl) amines, tri(cycloalkenyl) amines, substituted cycloalkenyl amines, disubstituted cycloalkenyl amine, trisubstituted cycloalkenyl amines, aryl amines, diaryl amines, triaryl amines, heteroaryl amines, diheteroaryl amines, triheteroaryl amines, heterocyclic amines, diheterocyclic amines, triheterocyclic amines, mixed di- and tri-amines where at least two of the substituents on the amine are different and are selected from the group consisting of alkyl, substituted alkyl, alkenyl, substituted alkenyl, cycloalkyl, substituted cycloalkyl, cycloalkenyl, substituted cycloalkenyl, aryl, heteroaryl, heterocyclic, and the like. Also included are amines where the two or three substituents, together with the amino nitrogen, form a heterocyclic or heteroaryl group.

### VII. Administration of Compositions

The preferred amount of the compounds of the invention is a therapeutically effective amount thereof which is also medically acceptable. Actual dosage levels of in the pharmaceutical compositions of the present invention may be varied so as to obtain an amount which is effective to achieve the desired therapeutic response for a particular patient, pharmaceutical composition, and mode of administration, without being toxic to the patient. The selected dosage level and frequency of administration will depend upon a variety of factors including the route of administration, the time of administration, the duration of the treatment, other drugs, compounds and/or materials used in combination with the compounds of the invention, the age, sex, weight, condition, general health and prior medical history of the patient being treated, and like factors well known in the medical arts. A physician having ordinary skill in the art can readily determine and prescribe the therapeutically effective amount of the pharmaceutical composition required.

Effective amounts can be determined, for example, by measuring increases in the immune response, for example, by the presence of higher titers of antibody, the presence of higher affinity antibodies, the presence of a desired population of immune cells such as memory cells to a particular antigen, or the presence of particular antigen specific cytotoxic T cells. Effective amounts also can be measured by a reduction in microbial load in the case of an infection or in the size or progression of a tumor in the case of cancer. An effective amount also may be reflected in a reduction in the symptoms experienced by a particular subject being treated.

Dosage may be adjusted appropriately to achieve desired drug levels, locally or systemically. Generally, daily doses of compounds will be from about 0.001 mg/kg per day to 1000 mg/kg per day. It is expected that doses in the range of about 0.1 to 50 mg/kg per day will be effective. In the event that the response in a subject is insufficient at such doses, even higher doses (or effective higher doses by a different, more localized delivery route) may be employed to the extent that patient tolerance permits. In one embodiment, each drug is administered one to four times daily for at least one day, at least 1-4 weeks, at least 1-11 months, or at least 1-10 years, and may even be for the life of the patient. Chronic, long-term administration will be indicated in many cases.

A variety of administration routes are available. The particular mode selected will depend of course, upon the particular drug selected, the severity of the disease state being treated and the dosage required for therapeutic efficacy. The methods of this invention, generally speaking, may be practiced using any mode of administration that is medically acceptable, meaning any mode that produces effective levels of the active compounds without causing clinically unacceptable adverse effects. Such modes of administration include oral, rectal, sublingual, topical, nasal, transdermal or parenteral routes. The term “parenteral” includes subcutaneous, intravenous, intramuscular, or infusion. Oral and intravenous routes are preferred. For administration by injection, conventional carriers well known to those of ordinary skill in the art can be used.

One preferred manner of administration for the conditions detailed above is oral, using a convenient daily dosage regimen which can be adjusted according to the degree of affliction. For such oral administration, a pharmaceutically acceptable, non-toxic composition is formed by the incorporation of any of the normally employed excipients, such as, for example, mannitol, lactose, starch, magnesium stearate, sodium saccharine, talcum, cellulose, sodium cross-carmellose, glucose, gelatin, sucrose, magnesium carbonate, and the like. Such compositions take the form of solutions, suspensions, tablets, dispersible tablets, pills, capsules, powders, sustained release formulations and the like.

Other delivery systems can include time-release, delayed release or sustained release delivery systems. Such systems can avoid repeated administrations of the conjugates of the invention, increasing convenience to the subject and the physician. Many types of release delivery systems are available and known to those of ordinary skill in the art. They include polymer based systems such as polytactic and polyglycolic acid, polyanhidrides and polycaprolactone; wax coatings, compressed tablets using conventional binders and excipients, and the like. Bioadhesive polymer systems to enhance delivery of a material to the intestinal epithelium are known and described in published PCT application WO 93/21906. Capsules for delivering agents to the intestinal epithelium also are described in published PCT application WO 93/19660.

A physician or veterinarian having ordinary skill in the art can readily determine and prescribe the effective amount of the pharmaceutical composition of mebendazole, cytochalasin E, deoxysappanone, nocodazole, paclitaxel, podofilox, podophyllotoxin acetate or vinblastine that is required to treat the condition. For example, the physician or veterinarian could start doses of the drug and increase or decrease the levels as required in order to achieve the desired therapeutic effect. One skilled on the art may rely on dosages used to treat other conditions. The effective amount of the compound may be one sufficient to reduce, inhibit, ameliorate, or delay at least one sign or symptom of the disease or condition (e.g., cell necrosis and apoptosis or organ failure). The amount of compound administered can be dependent upon the disease to be treated, the particular compound being employed, and the pharmacokinetics and pharmacodynamics of the drug in the subject being treated.

### EXEMPLIFICATION

The invention now being generally described, it will be more readily understood by reference to the following examples, which are included merely for purposes of illustration of certain aspects and embodiments of the present invention, and are not intended to limit the invention, as one skilled in the art would recognize from the teachings hereinabove and the following examples, that other DNA microarrays, cell types, agents, constructs, or data analysis methods, all without limitation, can be employed, without departing from the scope of the invention as claimed.

The contents of any patents, patent invention, patent publications, or scientific articles referenced anywhere in this invention are herein incorporated in their entirety.

### Example 1

We performed gene expression-based screening for mitochondrial biogenesis and cellular assays of mitochondrial function in mouse skeletal muscle cells. Approximately ˜2500 compounds were screened.

### Culture and Differentiation of Myoblasts in 384-Well Format

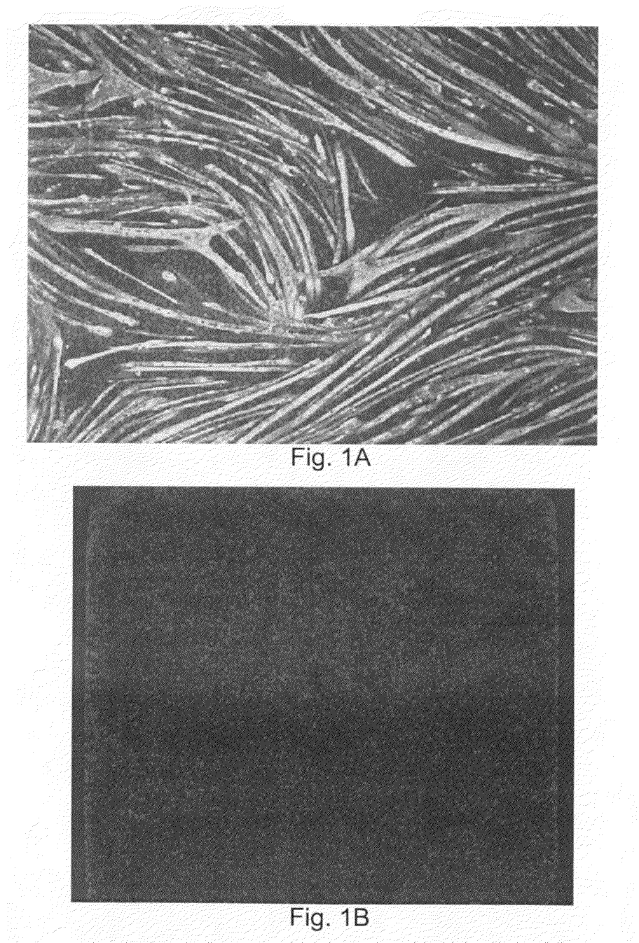

We have optimized protocols for growing and differentiating murine C2C12 myoblasts. These cells are simple to culture, can be differentiated into myotubes, and have been investigated in the context of mitochondrial biogenesis following electrical stimulation (Wu et al. 1999) and PGC-1α transduction (Connor et al. 2001). FIG. 1 shows myotubes in 384-well plate wells stained for nuclei with Hoechst (FIG. 1B) and for myotube morphology with anti-myosin heavy chain (FIG. 1A). The nuclei were counted using Axon ImageXpress automated imaging analysis. We detected 5313+/−384 nuclei per well, corresponding to a coefficient of variation (CV) of 7%.

### Cellular Assays of Mitochondrial Biogenesis and Function

Mitochondria are complex organelles that serve as the home for oxidative phosphorylation (OXPHOS), key steps of apoptosis, ROS homeostasis, and other key cellular pathways. Owing to this complexity, multiple measurements are necessary to characterize the state of mitochondrial function. We have developed several cell-based readouts of mitochondrial function and have adapted them to 384-well format. Here, we describe each assay and its reproducibility:

### Assay 1: Calcein Quantitation of Apoptosis

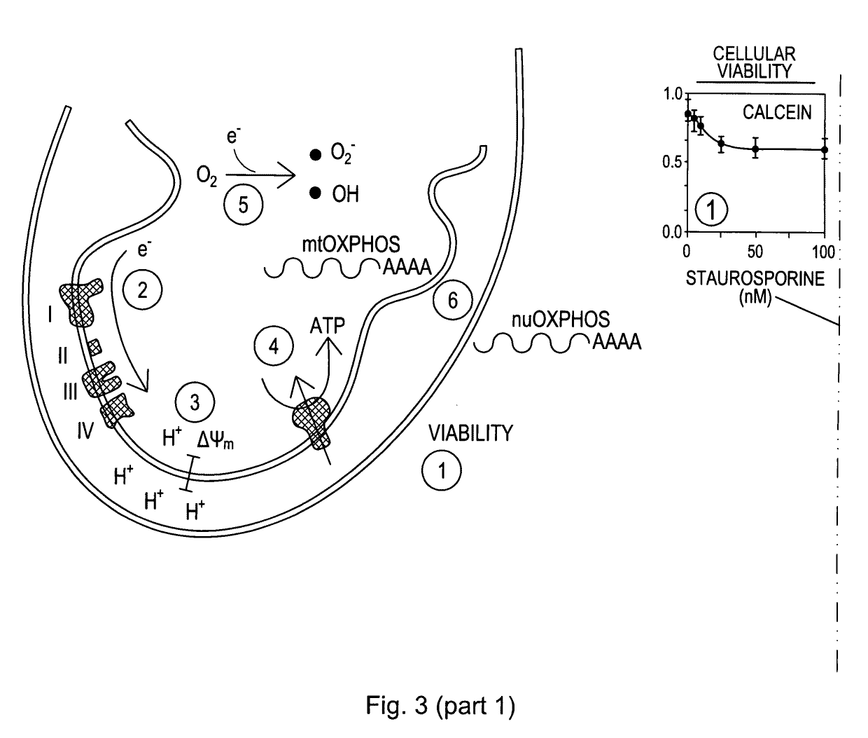

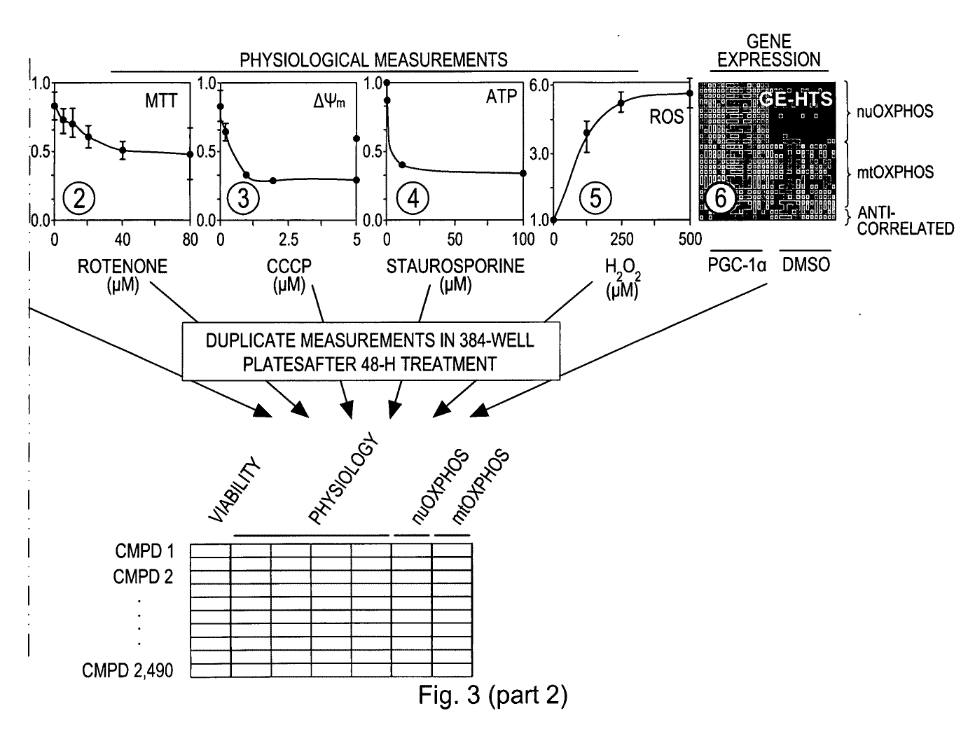

Mitochondria are often referred to as the gatekeepers of apoptosis (Wei et al. 2001) and we expect many compounds will induce apoptosis. Calcein stains are commercially available and provide fluorescent readouts of apoptosis. This assay is a simple add and read assay and we have adapted it to C2C12 myotubes with a CV of 8-13%. We can quantitate staurosporine-induced cell death in a dose dependent manner (FIG. 3-1).

### Assay 2: MTT Assay for Cellular Dehydrogenase Activity

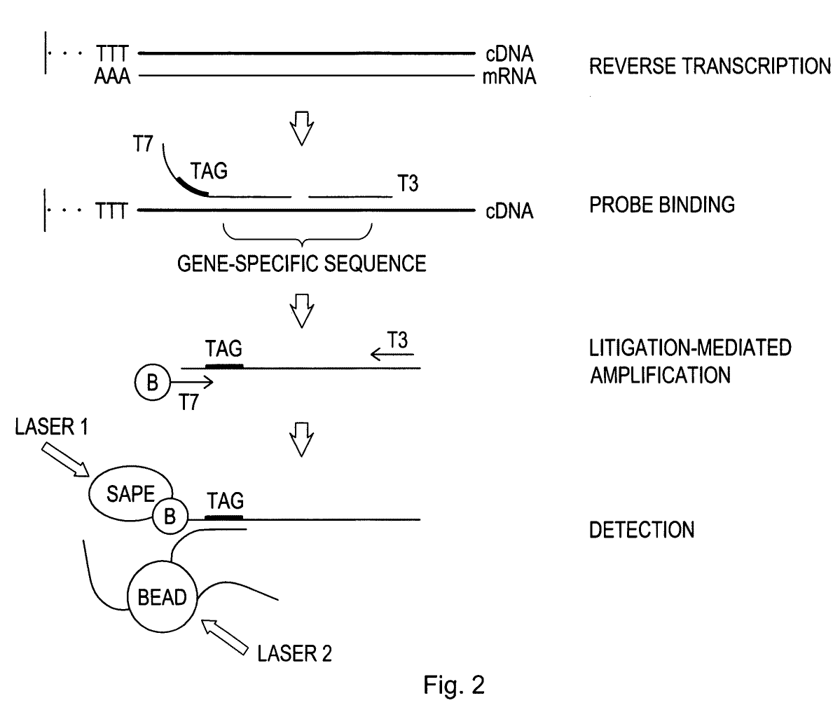

The cellular reduction of 3-(4,5-dimethylthiazol-2-yl)-2,5-diphenyltetrazolium Bromide (MTT), is a good indicator of cell viability and proliferation, as well as mitochondrial enzyme activity. Mitochondria are a likely site a site for MTT reduction, where MTT is converted to a colored formazan byproduct via a group of mitochondrial dehydrogenases, including NADH dehydrogenase, malate dehydrogenase, and succinic dehydrogenase. We incubated cells for 2 hours in medium to which MTT was added, and measured MTT reduction as a change in absorbance at 540 nm. Measurement of MTT activity is inhibited by the complex I inhibitor rotenone (FIG. 3-2).

### Assay 3: JC-1 Detection of Mitochondrial Membrane Potential

One of the mitochondrion's key bioenergetic parameters is its membrane potential (Ψₘ). We measured Ψₘ using JC-1, a lipophilic cation. JC-1 (5,5′,6,6′-tetrachloro-1,1′,3,3′-tetraethylbenzimidazolylcarbocyanine iodide) is a membrane-permeable probe that binds to mitochondrial membranes within cells and fluoresces green as an individual molecule (ex. 485/em. 530), but is converted to a red fluorescent form (ex. 530/em. 585) when it is internalized in a voltage-dependent manner across the mitochondrial inner membrane, forming so-called “J-aggregates”. The ratio of red to green signal is thus an indicator of Ψₘ. As shown in FIG. 3-3, the method readily detects depolarization induced by carbonyl cyanide m-chlorophenylhydrazone (CCCP), a mitochondrial uncoupler, with a CV of 7-13%.

### Assay 4: Fluorescent Detection of ATP

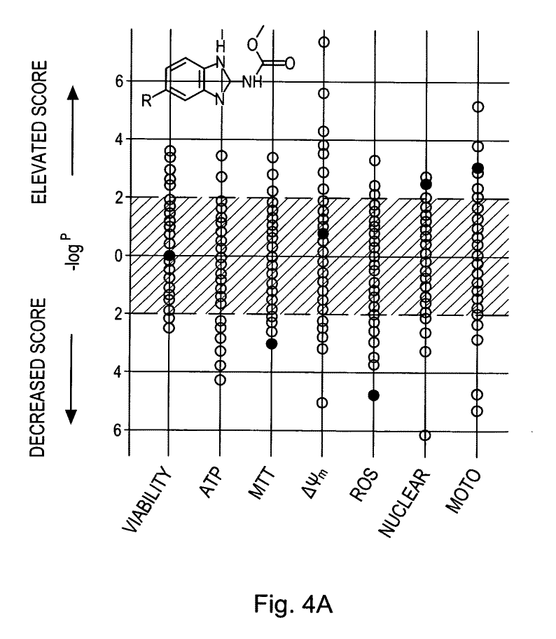

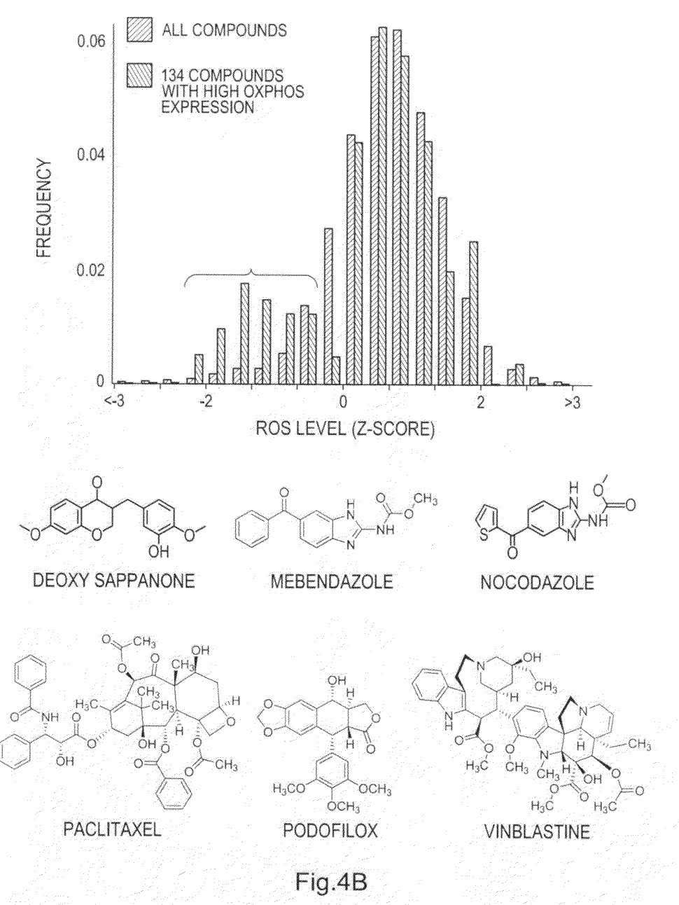

Over 90% of cellular ATP is generated by mitochondrial OXPHOS. Using a commercially available reagent called Cell-Titer Glo, we have been able to quantitate cellular ATP levels in 384-well format. This reagent allows quantitation in an “add-and-read” format; the lysis buffer is supplemented with recombinant luciferase and substrate, with cellular ATP providing the necessary energy for luminescence, which is read in 10 minutes on a plate reader. We estimated our CV to be 7-12%. (FIG. 3-4).

### Assay 5. Fluorescent Detection of Reactive Oxygen Species

Mitochondria are one of the primary sources of reactive oxygen species (ROS) and are elevated during injury to the electron transport chain. ROS are of outstanding relevance to diabetes since recent work from Houstis et al. has suggested they play a causal role in the development of insulin resistance (Houstis et al. 2006). We have adapted a commercially available ROS assay called 5-(and-6)-chloromethyl-2′,7′-dichlorodihydrofluorescein diacetate, acetyl ester (CM-H₂DCFDA) to 384-well format. The dye freely enters the cell and is retained intracellularly upon cleavage by cellular esterases. Once the dye is oxidized, it is converted to green fluorescent form. FIG. 3-5 depicts the results of the assay in response to increasing doses of hydrogen peroxide. Replicate measurements indicate that our CV is 6-8%.

### Assay 6. Gene Expression-Based High-Throughput Screening (GE-HTS) for Mitochondrial Biogenesis

To complement these physiological assays, we also performed gene expression-based high-throughput screening (GE-HTS) to profile transcripts associated with nuclear and mitochondrial DNA (mtDNA) expression of genes related to oxidative phosphorylation (OXPHOS). GE-HTS is a technique that uses a gene expression signature itself as the “readout” in high-throughput screening. It has already been applied to cancer gene expression for the discovery of novel lead compounds (Stegmaier et al. 2004; Hieronymus et al. 2006; Peck et al. 2006). We have developed a GE-HTS assay corresponding to the OXPHOS gene expression signature that we and others have reported in human diabetes (Mootha et al. 2003; Patti et al. 2003).

GE-HTS is a facile, high-throughput method that quantifies dozens of transcripts simultaneously. It is a multiplexed PCR strategy that combines ligation-mediated amplification with multicolored bead detection to identify and quantify transcripts of interest. We adapted GE-HTS to profile simultaneously all 13 mtDNA-encoded OXPHOS (mtOXPHOS) transcripts as well as 12 nuclear-encoded OXPHOS (nuOXPHOS) transcripts. These 12 nuOXPHOS transcripts include representatives from all five OXPHOS protein complexes and were selected because they capture virtually all of the variation in gene expression shown by the entire OXPHOS repertoire, as assessed by analysis of over 5,000 genome-wide microarrays. (Table 1) Of note, our GE-HTS assay also monitored transcripts that tend to be anticorrelated to OXPHOS expression or are invariant across many conditions as assessed by microarray assays, and thereby assist in data analysis. Together, our GE-HTS assay faithfully ‘tags’ the expression of the entire OXPHOS system. FIG. 3-6 illustrates the induction of OXPHOS genes by treatment with PGC-1a. Because the expression of OXPHOS genes is so highly correlated, measuring multiple transcripts increases the signal-to-noise ratio with which we can detect subtle effects of individual compounds.

Finally, the GE-HTS assay also provides a means to focus on the relationship between nuclear OXPHOS (nuOXPHOS) and mtDNA OXPHOS (mtOXPHOS) transcription. Chemical compounds that influence the two sets of genes in a coordinated manner can be identified, as can those which decouple the coordination between the two genomes.

To perform GE-HTS, transcripts of genes isolated from a sample are bound to poly-dT. Two nucleic acid primers to each of 13 mitochondrial-DNA-encoded OXPHOS (mtOXPHOS) transcripts and each of 12 nuclear-encoded OXPHOS (nuOXPHOS) transcripts are designed. One primer, the upstream primer, binds to the 5′ end of the target sequence. The upstream primer contains nucleotides that complement the target sequence, linked to nucleotides of a tag sequence, which are in turn linked to nucleotides that complement the universal primer (T7) site. A second primer, the downstream primer, binds to the 3′ end of the target sequence. The downstream primer contains nucleotides that complement the target sequence, linked to nucleotides that complement the universal primer (T3) site, and is phosphoryated. The SEQ ID numbers and sequences for the upstream and downstream primers used in the examples of this invention are listed in Table 10. After a pair of primers has bound to the target sequences, the pair is elongated and annealed to produce a copy of the target. The copy now contains the complement of the target sequence, the tag sequence, and both universal primer sites. An additional round of amplification is performed on the annealed copy, using a T3 primer and a T7 primer that has been biotinylated, to produce amplification products that contain the target sequence, a tag sequence, and are biotinylated. The amplification products are hybridized against a pool of colored beads, each of which has a nucleic acid that is complementary to one of the tag sequences. The amplification products are further incubated with streptavidin-phycoerythrin, which confers a fluorescent label on the biotin. The colored beads bound to the amplification products are subjected to flow cytometry, which serves to identify which tag sequences—and corresponding target genes—have been amplified. Fluorescently labeled amplification products are further quantified to determine the levels of target gene produced.

For these experiments, Applicants selected as tags nucleic acid sequences from a set of 35 (Table 9), but Applicants note that tags known in the art, or other nucleic acid sequences not present in the target sequences, could be used. In addition, the universal primers T3 and T7 were used, but any other universal primer or any other nucleic acid sequence not present in either the target sequence or the tag sequence could be used. In addition, biotin and streptavidin-phycoerythrin were used as binding moieties and phycoerythrin was used to confer a fluorescent label on the biotin. Any other binding moiety and fluorescent label known in the art could be substituted.

### Assay 7. Immunofluorescent Detection of Cytochrome c Protein Content.

Cytochrome c is a water-soluble mitochondrial protein found in the inner mitochondrial membrane. Cytochrome c acts as an electron carrier in oxidative phosphorylation, and also plays a crucial role in apoptosis, through activation of caspase 9 and downstream caspases. We developed an immunofluorescence-based method for detecting cytochrome c. Data from our screen for cytochrome c protein expression was included in a compendium of all of our results from the 7 assays, although we excluded it from subsequent analyses owing to the high coefficient of variation.

### Chemical Screening of 2490 Compounds and Bioactives

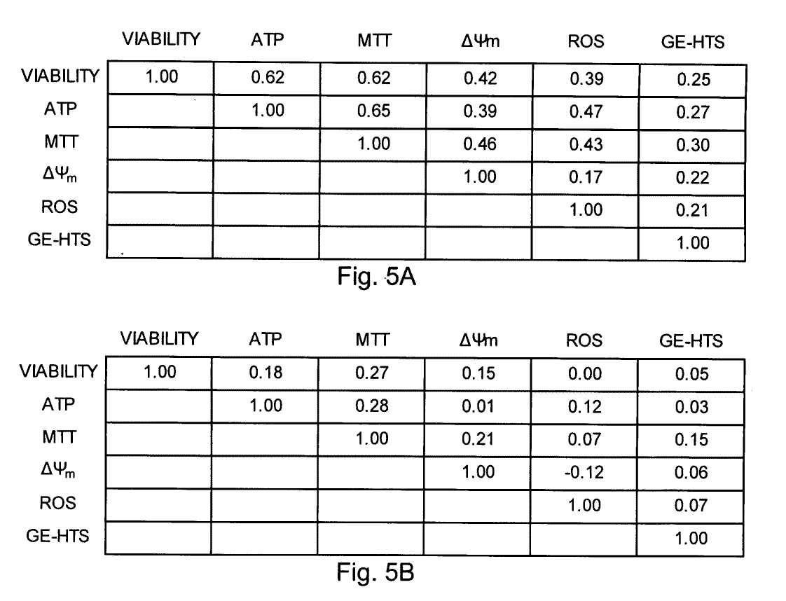

We have obtained a collection of 2490 compounds from the Spectrum Collection and the Prestwick Chemical Library, including ˜40% of all FDA approved drugs. We performed the viability, physiology and gene-expression assays in duplicate in differentiated C2C12 myotubes following 48-hour treatment with each of 2,490 compounds. Our chemical library consists of known bioactives, two-thirds of which are marketed drugs. Using a scoring algorithm dependent upon the distribution of mock-treated (DMSO) wells, we arrived at a normalized score for each assay in each well (Table 2). A compendium of our results includes data from our screen for cytochrome c protein expression, though we excluded it from subsequent analyses owing to the high coefficient of variation. Correlation analysis indicated that our remaining readouts (one for viability, four for OXPHOS physiology and one for OXPHOS gene expression) provide complementary information (FIG. 5).

Unlike traditional approaches for studying mitochondrial function, our improved screening method enables us to track systematically how changes in nuclear and mitochondrial OXPHOS gene expression are coupled to mitochondrial physiology over thousands of perturbations. We used this approach to explore three problems focused on mitochondrial biology, drug toxicity and the identification of novel therapeutics.

### Example 2

### Identification of Lead Compounds for Treating Mitochondrial Disorders

The GE-HTS assay is of particular interest to us since it is specifically assaying for the gene expression signature of human diabetes (Mootha Nat. Genet. 2003). We queried our compendium to identify compounds that might be capable of elevating OXPHOS expression while reducing ROS accumulation, as we and others have recently shown that a decline in OXPHOS gene expression and an elevation in ROS generation are associated with type 2 diabetes (Mootha Nat. Genet. 2003), neurodegeneration and aging.

We selected the top 22 compounds (˜1% of tail distribution) that promote the OXPHOS gene expression signature and re-tested these compounds in quadruplicate at four decreasing doses (10 μl, 0.1, 0.01 μM). Sixteen of 22 compounds reproduced the increase in expression signature at p<0.05 significance level (Kruskal-Wallis test, Dunn's multiple comparison post-test) at screening dose and 8 of these showed significance at multiple doses. Table 3 lists the top compounds identified in the screen.

In addition, we used two computational strategies to spotlight compounds that elevate OXPHOS expression while reducing ROS accumulation. First, we developed a simple analytical strategy to determine whether any structurally related set of compounds might boost OXPHOS expression while also suppressing ROS accumulation. This strategy involves organizing all compounds based on structural similarity and then asking whether members of a cluster had concordant scores in a given assay (Table 4). In FIG. 4a, the gray data points spotlight a single cluster of compounds, who share the chemical scaffold shown at the top. This cluster is significant for the desired activity, as measured by six separate assays. The advantage of this strategy is that individual compounds might show a subtle response not detectable in a primary screen with duplicate measurements, whereas the grouped analysis provides added statistical power.

Second, in a complementary approach, we sought to identify individual compounds that promote OXPHOS gene expression while reducing ROS levels. The advantage of this method is that it can reveal structurally unrelated compounds that individually exert large effects in the two assays of interest. We focused on the compounds that showed an elevation of OXPHOS expression and a decrease in ROS levels (bracketed in histogram in FIG. 4b). The structure of the compounds is also shown in FIG. 4b.

Notably, both analytical strategies spotlighted microtubule modulators, including both a microtubule stabilizer (paclitaxel) and several destabilizers (mebendazole, nocodazole, podophyllotoxin and vinblastine) (see Table 5), as agents that boost OXPHOS expression while suppressing ROS levels. The second strategy also yielded deoxysappanone B, a natural product found in sappan wood, whose molecular mode of action is unknown and has not been previously linked to microtubule biology (see Table 6). The other microtubule inhibitors within the compound collection (colchicine and griseofulvin) did not display the same decrease in ROS levels, but did show a modest increase in OXPHOS expression.

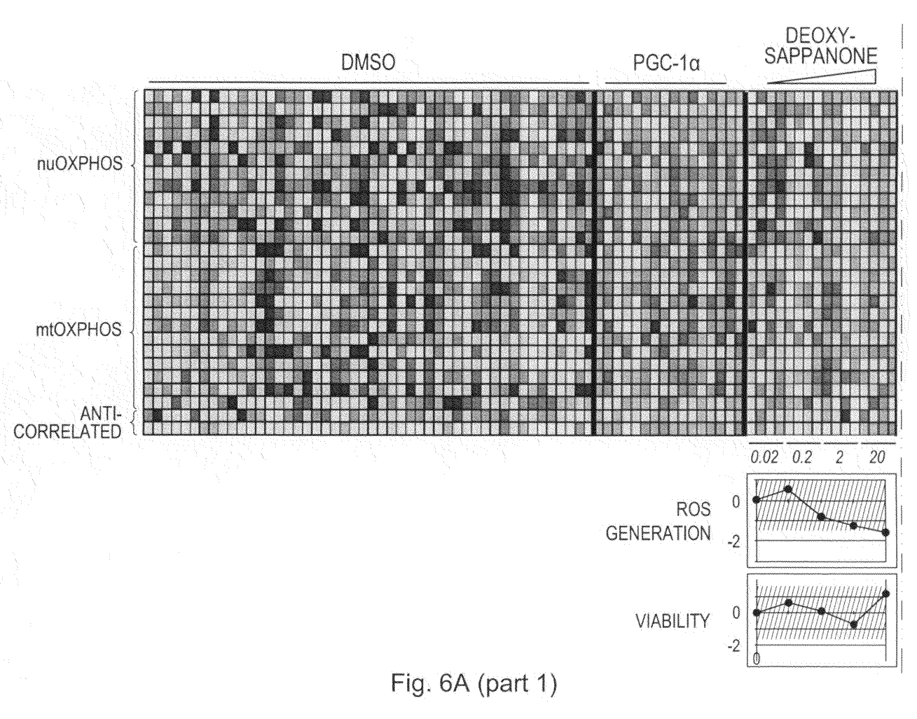

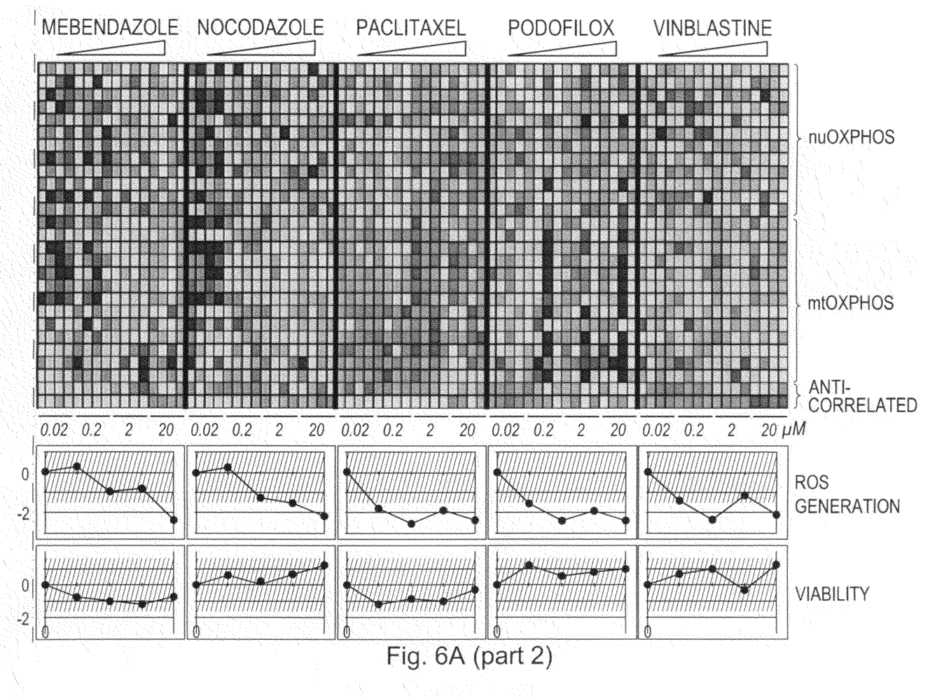

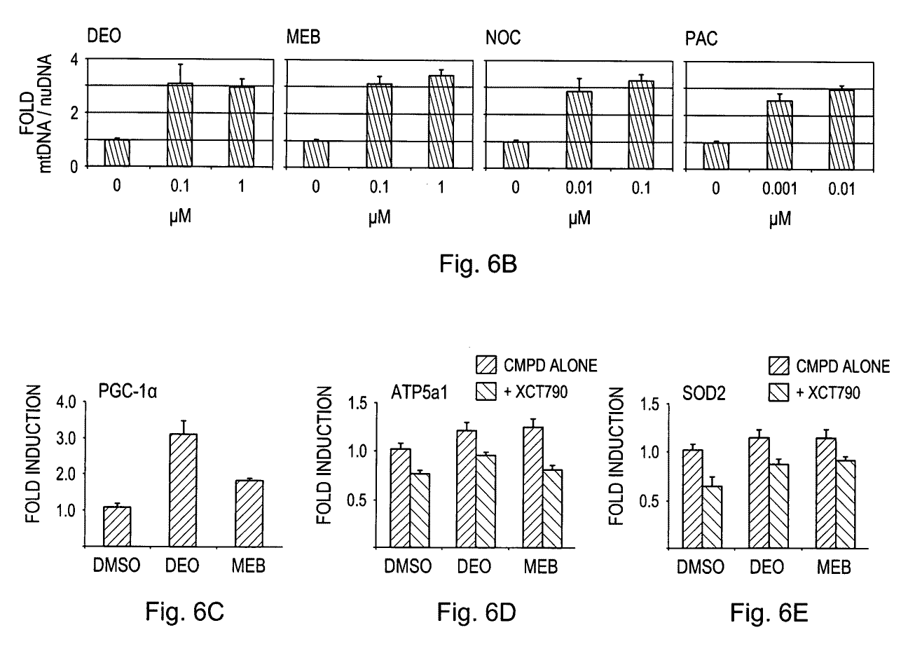

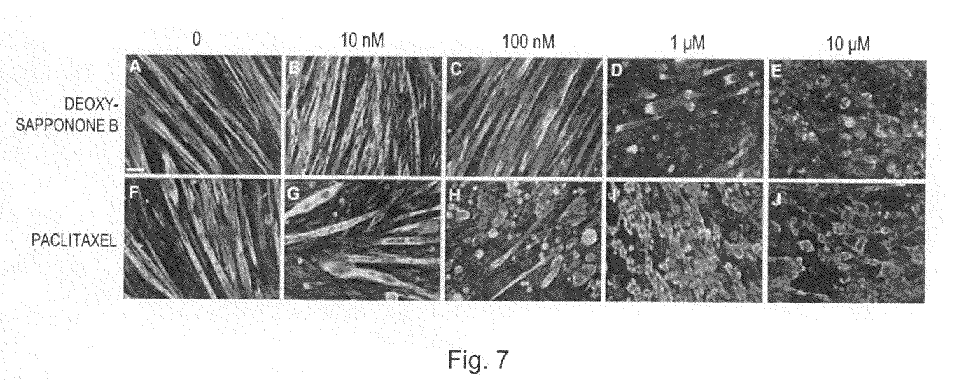

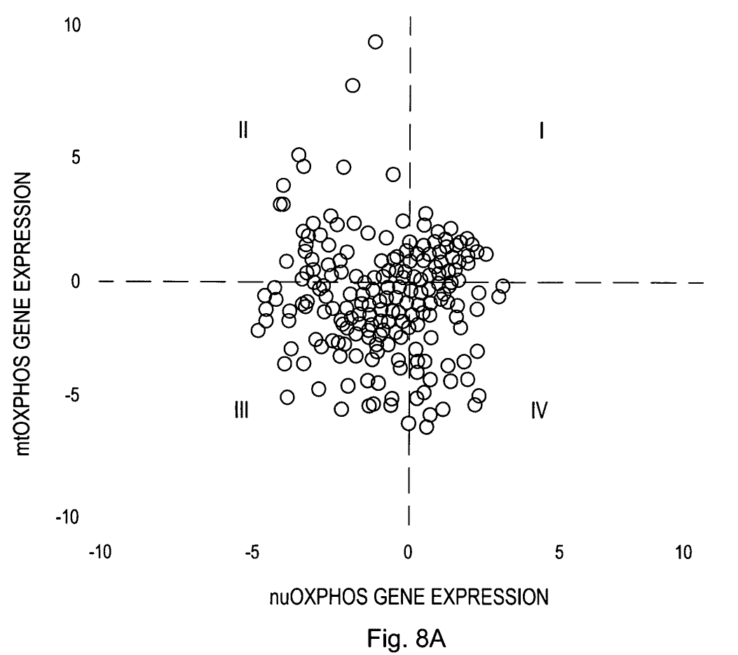

Next, we were interested in confirming these primary screening results and determining whether the effects on OXPHOS expression and ROS levels occur via shared or distinct mechanisms, and whether these were on-target or off-target effects of microtubule disruption. We therefore retested the microtubule modulators at a range of 20 nM to 20 μM (FIG. 6a). Treatment with either deoxysappanone B, mebendazole, nocodazole, podophyllotoxin or vinblastine increased OXPHOS expression and decreased ROS levels at the same dose of 2 μM. In contrast, paclitaxel showed effects in the two assays at 20 nM, suggesting a shared mechanism for OXPHOS expression and ROS level. Notably, at these doses, these compounds did not decrease cell viability (FIG. 6a), indicating that the decline in ROS is not simply a reflection of overt cytotoxicity. We also imaged tubulin immunofluorescence after treatment with deoxysappanone B and paclitaxel, two compounds that showed distinct potencies. For both compounds, the potency required for microtubule disruption was the same as that required to affect OXPHOS expression and ROS level (FIG. 7). To our knowledge, deoxysappanone B has not previously been linked to microtubule inhibition, but it now has been predicted to do so and the prediction validated by this study. Given that structurally and mechanistically diverse microtubule modulators increased OXPHOS gene expression, decreased cellular ROS and disrupted microtubules with equivalent potencies, it is likely that these effects are directly related to inhibition of microtubules, and not due to an off-target effect.

Because mtDNA replication and transcription are often coupled, we sought to determine whether any of these compounds promoted mtDNA replication. At the concentrations tested, several of these microtubule modulators—but not podophyllotoxin or vinblastine—increased mtDNA copy number approximately threefold (FIG. 6b).

We sought to determine the transcriptional mechanism by which microtubule inhibition might promote OXPHOS expression and mtDNA replication while suppressing ROS. We hypothesized that these changes might be occurring via PGC-1α, a transcriptional coactivator that regulates mitochondrial biogenesis in muscle and whose transcriptional program is diminished in type 2 diabetes. Consistent with this hypothesis, both mebendazole and deoxysappanone B induced the expression of Ppargc1α (which encodes PGC-1α) by approximately threefold (FIG. 6c). We have previously shown that the transcription factor ERRa serves as a key transcriptional partner of PGC-1α to drive OXPHOS expression in muscle, and that disruption of ERRa with the selective inverse agonist XCT790 suppresses PGC-1α-induced OXPHOS expression. Therefore, we tested whether XCT790 is capable of inhibiting compound-induced transcription. We observed that both mebendazole and deoxysappanone B increased the expression of a nuclear OXPHOS gene, Atp5a1, by 20%, and that this increase was completely inhibited by XCT790 (FIG. 6d), further suggesting a PGC-1α-dependent mechanism of compound activity. The mitochondrial ROS scavenger MnSOD is downstream of the same PGC-1α-ERRα pathway and we observed decreased cellular ROS levels after treatment with these small molecules. We also tested the effects of the compounds on MnSOD. A similar increase in MnSOD levels, which was suppressible by XCT790, was observed with these compounds (FIG. 6e). These results suggest that microtubule modulators both activate OXPHOS transcription and reduce cellular ROS levels in a manner dependent on PGC-1α and ERRα.

At a molecular level, we have uncovered an unexpected link between microtubule disruption and an increase in PGC-1α/ERRα-mediated OXPHOS gene expression. Although changes in mitochondrial staining and morphology have been associated with microtubule inhibitors, no studies have specifically documented their effects on OXPHOS expression and ROS levels. It is possible that interactions between the cytoskeleton and the mitochondrion are important in integrating cellular homeostasis throughout the cell cycle. As many of these microtubule modulators are used for treating cancer, our results may enhance understanding of the metabolic basis of chemotherapeutic action. Our studies also raise the possibility that manipulation of the microtubule pathway may reverse the gene-expression and ROS signatures associated with common degenerative diseases and that these may represent therapeutic targets.

### Example 3

### Exploring Cross-Talk Between Nuclear and Mitochondrial Genomes

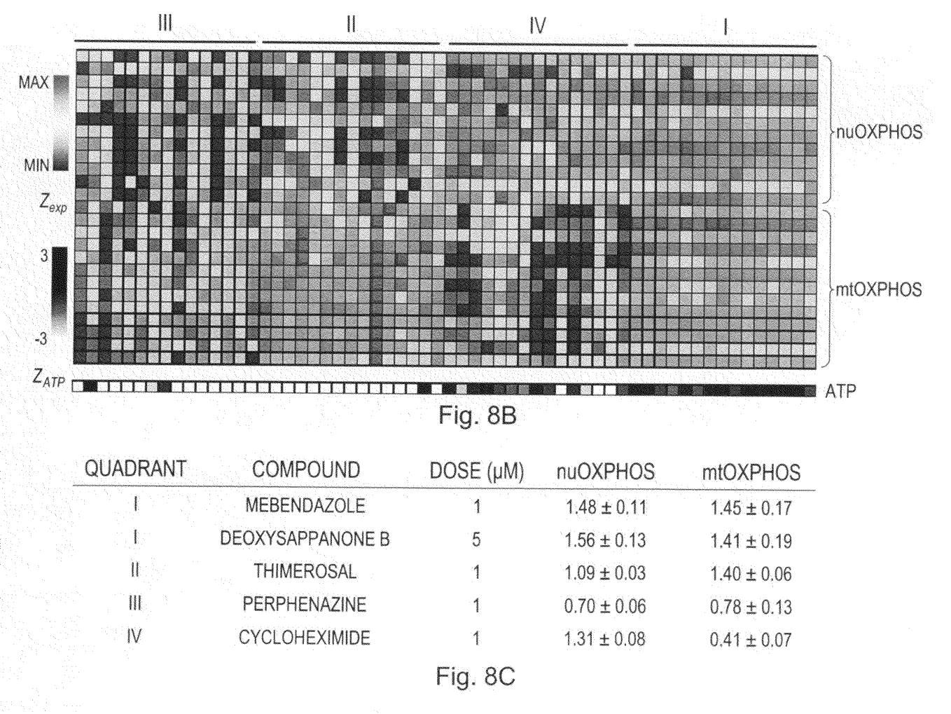

We used the compendium of assay results to identify the cellular signals involved in coordinating nuclear OXPHOS (nuOXPHOS) and mtDNA OXPHOS (mtOXPHOS) transcription. Expression of OXPHOS genes from the two genomes must be tightly coupled to maintain energy homeostasis in the mitochondrion. Moreover, although OXPHOS expression can change in human diseases, it is often unclear whether the changes are primary or reactive and how these changes relate to cellular physiology. We therefore focused on the relationship between nuOXPHOS and mtOXPHOS transcripts across the chemical perturbations. As expected, the majority of compounds influence the two sets of genes in a coordinated manner (FIG. 8a). However, we identified some compounds that decouple the coordination between these two genomes (FIG. 8b and Table 7), a subset of which we confirmed with follow-up dose response curves and RT-PCR analysis (FIG. 8c). Specifically, we discovered that the eukaryotic protein synthesis inhibitors emetine, anisomycin and cycloheximide preferentially increase nuOXPHOS expression, implying that translational control might be important in coordinating the two genomes. Follow-up studies revealed that 1 μM cycloheximide elevated nuOXPHOS 1.3-fold but decreased mtOXPHOS 2.4-fold (FIG. 8c) Notably, we found that nuOXPHOS expression, but not mtOXPHOS expression, correlated strongly with cellular ATP levels (FIG. 8b) To determine whether nuOXPHOS expression drives the changes in ATP levels, or reacts to changes in ATP levels, we performed follow-up time-course analyses with 20 μM perphenazine, a compound that decreased nuOXPHOS expression. Whereas nuOXPHOS expression declined significantly (21%, t-test, P=0.004) within the first hour of treatment, cellular ATP levels remained unchanged (0.6%, t-test, P=0.84) at these early time points. At later time points, however, ATP levels dropped significantly (8 h: 11% decrease, t-test, P=1.4×10⁻⁵, 24 h: 27% decrease, t-test, P=6.3×10²²), suggesting that the decline in nuOXPHOS expression precedes and drives the decline in cellular ATP levels.

Our compendium is the first to interrogate the expression of both the nuclear genome and mtDNA. Although we show that the bulk of compounds coordinately regulate expression from both genomes, we found that eukaryotic protein synthesis inhibitors disrupt cross-talk between these two genomes. Similar to the demonstration that the calcium ionophore A-23187 can elevate nuOXPHOS while decreasing mtOXPHOS, we now have identified an array of chemical tools to investigate whether protein synthesis inhibitors also disrupt the nuclear-to-mitochondrial genome cross-talk via known pathways or through one or more novel mechanisms.

### Example 4

### Exploring the Mitochondrial Basis for Drug Toxicity

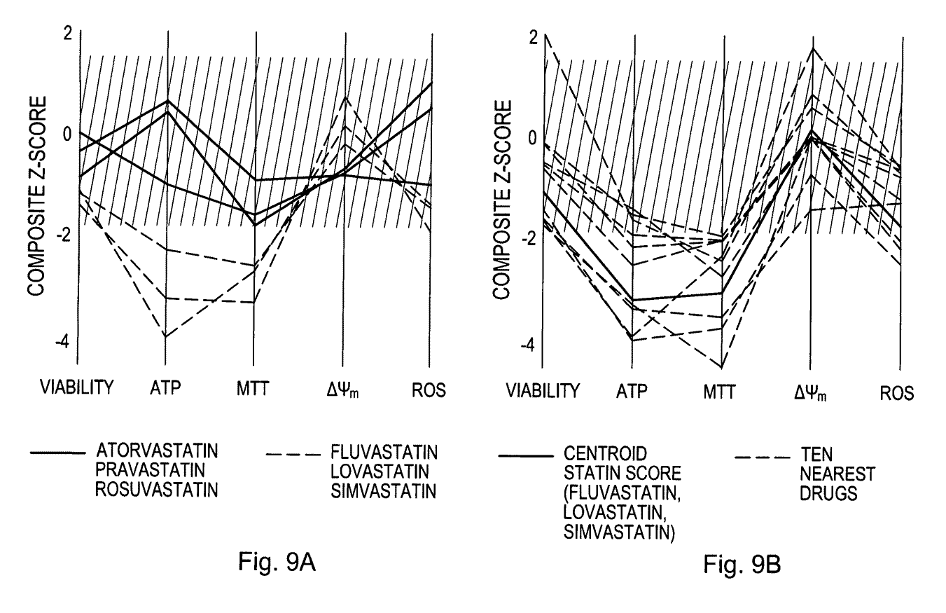

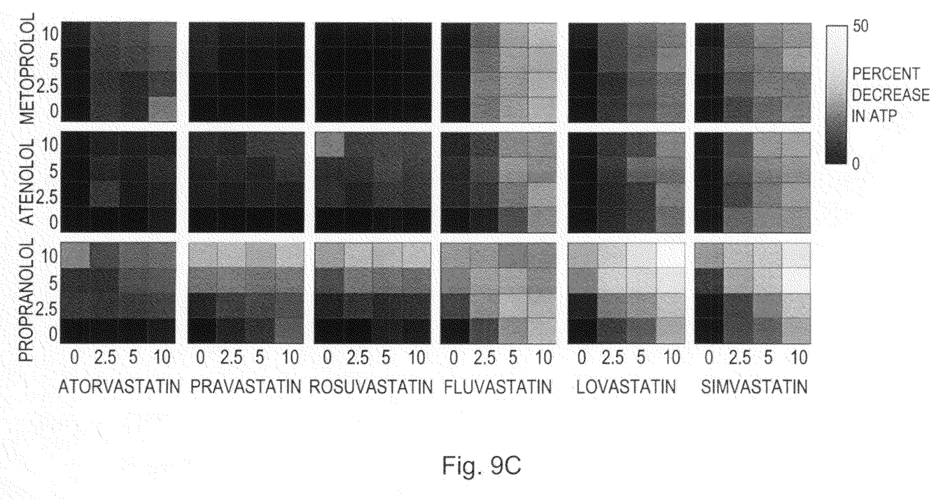

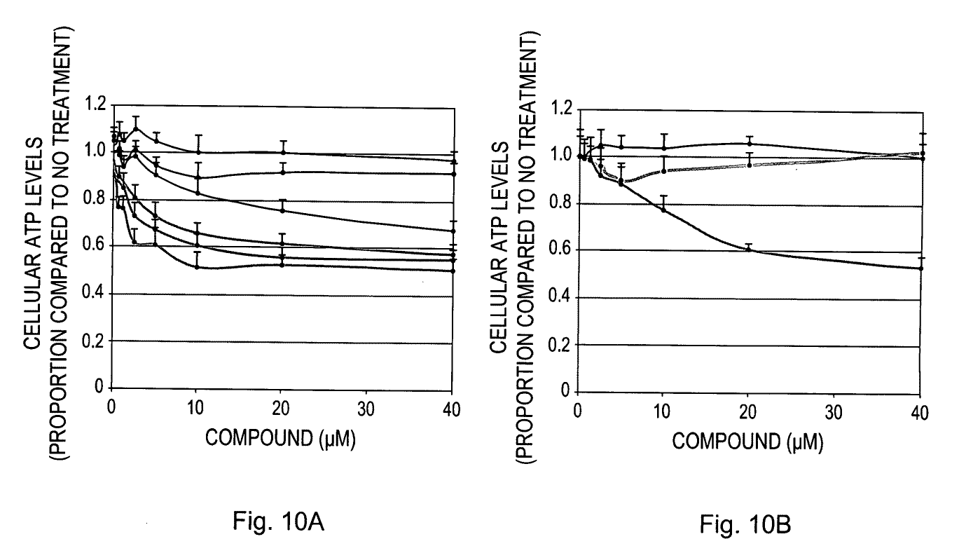

To probe the role of mitochondria in human drug toxicity, we focused on the statins-HMG-CoA reductase inhibitors taken by nearly 100 million patients worldwide. Statins are associated with a 0.1-0.5% incidence of myopathy, believed to be caused by ubiquinone depletion, which can block electron transport. However, clinical and epidemiological studies of the association between statins and myopathy have produced conflicting results. Of the six statins present in our screening collection, three (fluvastatin, lovastatin, simvastatin) produced strong decreases in cellular ATP levels and MTT activity (FIG. 9a). Previous studies showed that lovastatin and simvastatin reduce MTT activity and ATP levels, consistent with our high-throughput screening results. To eliminate the possibility that we uncovered two classes based merely on potency, we measured cellular ATP levels over doses ranging up to 40 μM. We observed the same segregation of effects, with atorvastatin, pravastatin and rosuvastatin showing little to no effect on cellular or mitochondrial ATP levels (FIG. 10).

To determine whether this profile might represent a signature of drug-induced myopathy, we established a centroid profile for the three mitochondria-active statins (fluvastatin, lovastatin and simvastatin) and sought to identify other clinically used drugs with a similar assay profile. The ten nearest-neighbor drugs to the centroid statin profile (FIG. 9b) were amoxapine, cyclobenzaprine, propranolol, griseofulvin, pentamidine, paclitaxel, propafenone, ethaverine, trimeprazine and amitriptyline. Notably, five of these compounds (amoxapine, propranolol, griseofulvin, pentamidine and paclitaxel) have also been associated with skeletal muscle myopathy or myalgia, a strikingly high proportion in comparison to the small fraction of all FDA-approved drugs believed to be associated with this side effect. This suggests that the drug profile might be indicative of myopathy or myalgia. Further examination of the screening data revealed that two electron transport chain inhibitors—β-dihydrorotenone (a complex I inhibitor) and antimycin A (a complex III inhibitor)—were among the 16 nearest-neighbor compounds to this assay profile, which provides mechanistic insight into this profile. Together, the data support the idea that myopathy induced by these five other drugs could be mitochondrial in origin.

Notably, one of these nearest-neighbor drugs is propranolol, a widely used antihypertensive agent. Follow-up experiments confirmed that propranolol, but not other selective β-1 blockers, decreases cellular ATP levels in a dose-dependent manner (FIG. 10). Because many patients take both a statin and a β-blocker for cardioprotection, we tested whether the two drugs might interact to cause toxicity. We thus assessed cellular ATP levels after treatment with all possible combinations of the six statins in our collection and three β-blockers (atenolol, metoprolol and propranolol), with all concentrations falling between 2.5 and 10 μM (FIG. 9c). Although neither atenolol nor metoprolol showed an effect either alone or in combination with any statin, propranolol had an additive effect on statin-induced decrease in ATP levels, as determined using the Bliss independence model (FIG. 9c). Our screening compendium and follow-up experiments (FIG. 9c) thus raise the potentially important hypothesis that patients on a combination of propranolol and one of the three statins (fluvastatin, lovastatin, simvastatin) might be at a higher risk for developing myopathy or myalgia. The additive interaction we reveal between the statins and propranolol suggests that patients taking both statins and propranolol might be at increased risk for developing skeletal muscle myopathy or myalgia. Because many patients with heart disease are likely to be on this drug combination, our hypothesis can be tested easily and may help to account for the conflicting reports on skeletal muscle myopathy associated with statins.

### Example 5

### Measurement of Glucose Uptake After Paclitaxel Treatment

For 3 hour paclitaxel treatment, differentiated myotubes were pre-incubated in serum-free DMEM for 1.5 hours followed by 2.5 hour treatment with 1 nM or 1 μM paclitaxel in serum-free DMEM. For 30 minute paclitaxel treatment, differentiated myotubes were pre-incubated in serum-free DMEM for 4 hours. Cells in 12 well dishes were then washed twice with KRH (140 mM NaCl, 5 mM KCl, 2.5 mM MgSO₄, 1 mM CaCl₂, 20 mM HEPES) and incubated with pre-warmed KRH (690 ul) containing 1 nM or 1 μM paclitaxel at 37° C. for 30 min. After this period, tritiated 2-deoxyglucose (2DG) and unlabeled 2DG (total vol. 50 μl) were dispensed into each well for a final concentration of 0.5 μCi/ml and 0.1 mM respectively. Cells were incubated for an additional 5 min. at 37° C. and the reaction was stopped by placing the dish immediately on ice followed by addition of ice-cold 500 μl phloretin-PBS (0.08 mg/ml) solution per well. Cells in each well were then washed twice with ice-cold phloretin-PBS (0.08 mg/ml) solution. The plate was then dried, and 740 ul of digitonin release buffer (100 mg/ml Mannitol, 1 mg/ml digitonin) was applied to each well. After 10 min. at room temperature, 670 ul from each well was counted in a scintillation counter. Results of the glucose uptake measurements are presented in Table 8.

### Materials and Methods:

Cell culture. C2C12 myoblasts (ATCC) were grown in Dulbecco's Modified Eagle's Medium (DMEM, Mediatech) supplemented with 10% (vol/vol) FBS and antibiotics (100 μg/ml penicillin/streptomycin mix) in a humidified atmosphere at 37° C. with 5% CO₂. Differentiation into myotubes was induced at 80% density on day 0 by changing the medium to DMEM supplemented with 2% (vol/vol) horse serum.

Cell-based high-throughput screening. For all screening, 4,000 C2C12 myoblasts per well were seeded into either black or white 384-well optical-bottom plates (Nunc) at 50 μl per well. On day 4 of differentiation, 100 nl of each compound was pin-transferred in duplicate into fresh medium with a steel pin array, using the CyBi-Well robot (CyBio). To increase the number of mock-treated wells included in the control distribution, we added an additional plate containing DMSO alone. Compound-treated plates were incubated at 37° C. for 48 h. All cell-based assay measurements were performed using the EnVision plate reader (PerkinElmer). The coefficient of variation for each of these assays was estimated to be less than 15%. All data has been deposited in ChemBank: see the World Wide Web at chembank.broad.harvard.edu/assays/view-project.htm?id=1000453.

Calcein viability assay. Medium was aspirated from plates, and 30 μl per well 1 μM calcein-AM (Molecular Probes) in phenol red-free medium was added. Plates were incubated for 1 h at 37° C. and washed three times with 50 μl per well PBS. Fluorescence was measured at excitation and emission wavelengths (ex/em) of 485 nm/530 nm.

JC-1 mitochondrial membrane potential assay. Upon depolarization, the JC-1 dye is converted from a diffuse green form to red fluorescent J-aggregates. The ratio of red to green fluorescence serves as a readout of the mitochondrial membrane potential. Medium was aspirated from plates, and 20 μl per well 3.25 μM JC-1 (Molecular Probes) in phenol red-free medium was added. Plates were incubated for 2 h at 37° C. and washed three times with 50 μl per well PBS. Fluorescence was measured first at ex/em 530 nm/580 nm (‘red’) and then at ex/em 485 nm/530 nm (‘green’).

Assay for cellular ATP levels. 20 μl per well CellTiterGlo reagent (Promega) was added to 20 μl per well of cell culture medium. Plates were agitated for 2 min and incubated for 10 min at room temperature (22-24° C.) before luminescence was measured.

MTT assay. Medium was aspirated from plates, and 50 μl per well 0.5 mg/ml MTT in phenol red-free medium was added. Plates were incubated for 2 h at 37° C., and this was followed by aspiration of MTT solution, addition of 50 μl per well DMSO to dissolve formazan crystals, and incubation at 37° C. for 30 min. After incubation, plates were equilibrated to room temperature for an additional 20-30 min. Absorbance was measured at 540 nm.

Reactive oxygen species assay. Medium was aspirated from plates, and 20 μl per well 10 μM CM-H₂DCFDA (Molecular Probes) in phenol red-free medium was added. Plates were incubated for 1 h at 37° C. and washed three times with 50 μl per well PBS. Fluorescence was measured at ex/em 485 nm/530 nm.

Cytochrome c protein detection. Cells were fixed with 3.7% (vol/vol) formaldehyde in PBS for 30 min and then washed with TBS containing 0.1% (vol/vol) Tween-20 (TBST) and blocked with TBST+3% (wt/vol) BSA for 1 h at room temperature. Cytochrome c was detected by incubating the cells with primary antibody (Cell Signaling Technology; 1:100) overnight at 4° C., washing three times with TBST, and incubating with secondary antibody (Alexa Fluor 488-conjugated anti-mouse IgG, Invitrogen; 1:250) for 1 h at room temperature. Plates were washed three times with TBST and fluorescence measured at ex/em 485 nm/530 nm.

Gene expression-based high-throughput screening. We adapted the GE-HTS assay to monitor both nuclear and mtDNA OXPHOS transcripts. To narrow down the list of potential genes from nearly 80 nuclear OXPHOS genes, we used a list of highly co-regulated OXPHOS genes that are coordinately expressed across tissues and are downstream of the PGC-1α transcriptional coactivator. From this list, we selected genes that showed the highest signal-to-noise ratio in the microarray analysis of PGC-1α overexpression in C2C12 myotubes representing all five OXPHOS complexes. We also selected two genes that are downregulated by PGC-1α with the best signal-to-noise ratio. As controls, we selected genes that showed the lowest signal (no treatment effect) and lowest noise (biological variation) in the PGC-1α overexpression data, as well as genes previously found to be invariant from the analysis of multiple microarray datasets. We selected control genes that span a wide range of expression levels to prevent biasing for abundant transcripts. The selected OXPHOS transcripts capture the bulk of the variation exhibited by the OXPHOS transcripts represented on over 5,000 publicly available mouse microarrays on the Affymetrix platform (data not shown).

From the list of OXPHOS genes and control genes for GE-HTS, we designed primer pairs with T7 and T3 universal primer sites, 40-bp target sequence split into two 20-bp sequences for each primer, and gene-specific barcode sequence attached to the 5′ primer according to the published assay specification. We selected 40-bp gene-specific target sequences that are not alternatively spliced using oligonucleotide sequences found in the Mouse Exonic Evidence-Based Oligonucleotide Chip (MEEBO, see the World Wide Web at alizadehlab.stanford.edu/). Full primer sequences are included in Tables 1 and 10.

The GE-HTS assay was performed as previously described. Because this assay measures the final amount of PCR products rather than providing a real-time measurement of gene expression, we adjusted the parameters in the original protocol so that the abundance of PCR products were within the linear range of the assay. We removed 20 μl of medium and added 25 μl of lysis buffer per well of a 384-well plate, and used 24 PCR cycles instead of the 29 cycles described. We used 32 DMSO-treated and 32 PGC-1α adenovirus-treated wells per 384-well compound plate, with one additional control plate containing 192 DMSO-treated wells, 32 GFP adenovirus-treated wells and 160 PGC-1α adenovirus-treated wells. The PGC-1α adenovirus-treated cells serve as a positive control for increased OXPHOS gene expression, as previously reported.

Tubulin immunofluorescence. On day 4 of differentiation, C2C12 myotubes were treated with each compound for 48 h and then fixed for 5 min in ice-cold 100% methanol. Cells were washed once in 50 μl PBSTB2 (PBS with 0.1% (vol/vol) Tween-20 and 2% (wt/vol) BSA) and blocked in PBSTB2 for 1 h at room temperature or overnight at 4° C. Cells were incubated with an anti-α-tubulin (Sigma-Aldrich) antibody, 1:1,000 in PBSTB2, for 1 h at room temperature, and then washed three times with PBSTB2. Cells were incubated with secondary antibody (Alexa 488-conjugated anti-mouse antibody, 1:500 in PBSTB2) (Molecular Probes) and Hoechst 33342 for 1 h at room temperature and then washed three times in PBSTB2. Cells were visualized using an automated microscope (IX-Micro, Molecular Devices).

Quantitative PCR of mtDNA and transcripts: mtDNA quantification. Mitochondrial DNA copy number was assessed by quantifying the abundance of the mitochondrial gene mt-Co1 (encoding Cox1) relative to the nuclear geneActb (encoding β-actin). DNA from cells were extracted using DNeasy (Qiagen) and quantified for mt-Co1 and Actb copy number using quantitative PCR (Applied Biosystems). The change in the mt-Co1/Actb ratio between the compound-treated and DMSO control cells represents the fold change in mtDNA copy number.

Gene expression. We extracted RNA using an RNeasy kit (Qiagen) and synthesized cDNA using a high-capacity cDNA reverse transcription kit (Applied Biosystems) with random hexamers, as described by the manufacturer. The cDNA was then used for real-time PCR quantification of products for mouse Atp5a1 (Mm00431960_ml), Sod2 (MnSOD; Mm01313000_m1) and Ppargc1a (Mm00447183_m1), with Hprt1 (Mm03024075_m1) serving as an internal control, using TaqMan gene-expression assays (Applied Biosystems).

Statistics: cell-based screening. Composite Z-scores reflecting compound performance as compared to a mock-treated (DMSO) distribution were calculated as described. (see also the World Wide Web at chembank.broad.harvard.edu/details.htm?tag=Help#screeningData).

GE-HTS. We first eliminated wells that failed the assay reaction by filtering out wells in which the raw expression value of Rps2 (a control gene) was 2 s.d. below the median DMSO control value for each plate. We normalized for plate-to-plate variation by scaling the per-well expression level of each gene to the median expression level of that gene in PGC-1α control wells on each plate. We set the median PGC-1α-treated expression value for each gene to 1, and then normalized for well-to-well variation by dividing the expression level of each OXPHOS gene by the average value of eight control genes for each well. This number represents the processed data value.

To score the expression levels of 12 nuclear- and 13 mitochondrial-encoded OXPHOS genes, we first weighted each gene by its ability to distinguish DMSO control wells from PGC-1α-treated wells. We calculated the signal-to-noise ratio of each gene using our PGC-1α-treated positive control and DMSO negative control, and multiplied the expression value of each gene per well by this signal-to-noise ratio. We then summed these weighted scores over nuclear-encoded or mitochondrial-encoded OXPHOS genes to derive one score each for expression within each genome. Composite Z-scores were calculated as described above.

Similarity between assay profiles. We used the cell-based composite Z-scores from the ATP, MTT, JC-1 and ROS assays to calculate the root-mean-square distance between performance vectors, as this statistic gives greater weight to values far from zero. We obtained centroid statin scores by taking the arithmetic mean of the composite Z-scores from these four assays.

Identifying structurally related small molecules. We used Pipeline Pilot (Scitegic) to perform K-means clustering of the molecules based on common and biologically intuitive chemical features (molecular weight, octanol-water partition coefficient, number of hydrogen bond donors and acceptors, and number of rotatable bonds). We set K to 624 to result in an average of 5 compounds per cluster. To detect enrichment for assay performance within each compound cluster, we performed the Mann-Whitney rank-sum test on each cluster in each assay.

TABLE 1

OXPHOS genes profiled by GE-HTS and 40-base
pair target sequences used for GE-HTS probes.

| | Gene Name Entrez | | |
|---|---|---|---|
| Type | GeneID number | Upstream (5′-3′) | Downstream (5′-3′) |
| mtOXPHOS | Mt-Atp6 | TTCAAGCCTACGTATTCACC | CTCCTACTAACCCTATATCT |
| | 17705 or 4508 | SEQ ID NO: 1 | SEQ ID NO: 2 |
| mtOXPHOS | Mt-Atp8 | TCACCAAAATCACTAACAAC | CATAAAAGTAAAAACCCCTT |
| | 17706 or 4509 | SEQ ID NO: 3 | SEQ ID NO: 4 |
| mtOXPHOS | Mt-Co1 | CACGACGCTACTCAGACTAC | CCAGATGCTTACACCACATG |
| | 17708 or 4512 | SEQ ID NO: 5 | SEQ ID NO: 6 |
| mtOXPHOS | Mt-Co2 | AACAAACGACCTAAAACCTG | GTGAACTACGACTGCTAGAA |
| | 17709 or 4513 | SEQ ID NO: 7 | SEQ ID NO: 8 |
| mtOXPHOS | Mt-Co3 | TAGGACTTTACTTCACCATC | CTCCAAGCTTCAGAATACTT |
| | 17710 or 4514 | SEQ ID NO: 9 | SEQ ID NO: 10 |
| mtOXPHOS | Mt-Cytb | CTAATACCTTTCCTTCATAC | CTCAAAGCAACGAAGCCTAA |
| | 17711 or 4519 | SEQ ID NO: 11 | SEQ ID NO: 12 |
| mtOXPHOS | Mt-Nd1 | TACTACTATCATCAACATTC | CTATGGATCCGAGCATCTTA |
| | 17716 or 4535 | SEQ ID NO: 13 | SEQ ID NO: 14 |
| mtOXPHOS | MtNd2 | TTCTTCCTTACAACCCATCC | CTCACTCTACTCAACCTCAT |
| | 17717 or 4536 | SEQ ID NO: 15 | SEQ ID NO: 16 |
| mtOXPHOS | Mt-Nd3 | TTACATTTCTATTATTTGAC | CTAGAAATTGCTCTTCTACT |
| | 17718 or 4537 | SEQ ID NO: 17 | SEQ ID NO: 18 |
| mtOXPHOS | Mt-Nd4 | ACTACGAACGGATCCACAGC | CGTACTATAATCATCGCCCG |
| | 17719 or 4538 | SEQ ID NO: 19 | SEQ ID NO: 20 |
| mtOXPHOS | Mt-Nd41 | ATTATAACTTCAGTAACTTC | CCTAAACTCCAACTCCATAA |
| | 17720 or 4539 | SEQ ID NO: 21 | SEQ ID NO: 22 |
| mtOXPHOS | Mt-Nd5 | CCTACTAATTACACTAATCG | CCACTTCTATAACAGCTATG |
| | 17721 or 4540 | SEQ ID NO: 23 | SEQ ID NO: 24 |
| mtOXPHOS | Mt-Nd6 | GAGATTCGTTGATGTATCAG | GTTGATGATGTTGGAGTTAT |
| | 17722 or 4541 | SEQ ID NO: 25 | SEQ ID NO: 26 |
| nuOXPHOS | Atp5a1 | AAAGGGTTACTCTTGTATTC | CTGATGTACAGAAATCACAT |
| | 11946 or 498 | SEQ ID NO: 27 | SEQ ID NO: 28 |
| nuOXPHOS | Atp5c1 | CTTGACTTTCAACCGCACCC GCCAGGCTGTCATCACAAAG | |
| | 11949 or 509 | SEQ ID NO: 29 | SEQ ID NO: 30 |
| nuOXPHOS | Atp5o | GCTGAAGAGCTTCCTGAGTC | CAAACCAAATACTCAAACTG |
| | 28080 or 539 | SEQ ID NO: 31 | SEQ ID NO: 32 |
| nuOXPHOS | Cox5b | CCAAAGGCAGCTTCACCCAC | CAAGGAAGACCCTAATCTAG |
| | 12859 or 1329 | SEQ ID NO: 33 | SEQ ID NO: 34 |
| nuOXPHOS | Cox7a2 | CCAATAAAGCAATCCTTAAC | CATTTTGTGTCTCCCTTTTC |
| | 12866 or 1347 | SEQ ID NO: 35 | SEQ ID NO: 36 |
| nuOXPHOS | Cyc1 | TTTCCCGGCCAGGCCATTCC | CATGGCTCCTCCCATCTACA |
| | 66445 or 1537 | SEQ ID NO: 37 | SEQ ID NO: 38 |
| nuOXPHOS | Hspc051 | TAAGGATGAGTTTCAAGTTG | CCGTTCACCGACCGCCAGTG |
| | 66152 or 29796 | SEQ ID NO: 39 | SEQ ID NO: 40 |
| nuOXPHOS | Ndufa5 | TCATATTCTGAAGCACTTTC | CTAAACATGCAGCCTATAGA |
| | 68202 or 4698 | SEQ ID NO: 41 | SEQ ID NO: 42 |
| nuOXPHOS | Ndufb5 | CTGTCCAAGAACAGTGTCTC | CCTCTAGTGGCAAGAAATGA |
| | 66046 or 4711 | SEQ ID NO: 43 | SEQ ID NO: 44 |
| nuOXPHOS | Sdhd | TTTAGACAAGTTCAATTTAG | GGAGTTCTCCTTCTTTCTGG |
| | 66925 or 6392 | SEQ ID NO: 45 | SEQ ID NO: 46 |
| nuOXPHOS | Uqcrb | CTGGATGGTTTTCGAAAGTG | GTATTATAATGCTGCAGGAT |
| | 67530 or 7381 | SEQ ID NO: 47 | SEQ ID NO: 48 |
| nuOXPHOS | Uqcrc1 | TCCCACACTACAACCGGATC | CGCACTGGCATGTTCTGGCT |
| | 22273 or 7384 | SEQ ID NO: 49 | SEQ ID NO: 50 |
| Control | Actb | TAAGTGGTTACAGGAAGTCC | CTCACCCTCCCAAAAGCCAC |
| | 11461 | SEQ ID NO: 51 | SEQ ID NO: 52 |
| Control | Aamp | GGGTGCGTCTTTCTATGTTG | GCGTTAGGTCTTTGAGGTTC |
| | 227290 | SEQ ID NO: 53 | SEQ ID NO: 54 |
| Control | Cenpb | GTCCAGCCACCCACGTGCTC | CTTTCCCAGCTTGAATTCAA |
| | 12616 | SEQ ID NO: 55 | SEQ ID NO: 56 |
| Control | Eefla1 | ATAACAATGCATCGTAAAAC | CTTCAGAAGGAAAGAATGTT |
| | 13627 | SEQ ID NO: 57 | SEQ ID NO: 58 |
| Control | Jund | CCGCCTCTCTACCCCCAGTC | CTGCCCGTGGCTGCCCCTTT |
| | 16478 | SEQ ID NO: 59 | SEQ ID NO: 60 |
| Control | Lsp1 | TGACCAACCCTCCAACTCTC | CTTCTCACCATCAGCTAAAG |
| | 16985 | SEQ ID NO: 61 | SEQ ID NO: 62 |
| Control | Rps2 | ACGGATCATCTTGTGAAAAC | CCACACCAGAGTCTCTGTTC |
| | 16898 | SEQ ID NO: 63 | SEQ ID NO: 64 |
| Control | Rps27a | TCGTAAGCACCTGGAAGATG | CCCGGACTTTGTCTGACTAC |
| | 78294 | SEQ ID NO: 65 | SEQ ID NO: 66 |
| PGC | Cyb5r3 | ACTCCATGCAGTCTTGAGTG | CCCTAAGTTGTCAGCCCAAC |
| 1α downreg. | 109754 | SEQ ID NO: 67 | SEQ ID NO: 68 |
| PGC- | Fh11 | TTCTCTGAAACGCAGGATTG | CCTCCTTAACTGTACTCTCC |
| 1α downreg. | 14199 | SEQ ID NO: 69 | SEQ ID NO: 70 |

TABLE 2
Chemical Screening of 2490 Compounds and Bioactives
3120 compound instances, 2490 unique compounds, ND refers to lack of sufficient mRNA in well

| Compound Name | Conc (μM) | Viability | ATP | MTT | ΔΨₘ | ROS | cyt c | GE-HTS | nucOX | mitoOX | ChemBank_ID | PubChem_SID |
|---|---|---|---|---|---|---|---|---|---|---|---|---|
| amiodarone | 6.2 | 1.332 | 1.212 | −0.418 | −0.531 | −0.119 | 0.079 | 0.040 | 0.158 | −0.216 | 26 | 11467557 |
| amiodarone | 20 | 0.499 | −0.282 | −0.339 | −0.092 | 0.119 | 0.336 | −1.187 | −0.895 | −1.513 | 26 | 11489629 |
| diazoxide | 17.34 | 0.608 | 1.679 | 0.109 | −0.769 | −0.439 | 1.122 | −0.115 | 0.050 | −0.474 | 35 | 11467235 |
| flufenamic acid | 14.22 | −0.666 | 0.487 | −0.731 | −0.780 | 1.304 | 0.763 | −1.130 | −1.270 | −0.673 | 41 | 11467351 |
| flufenamic acid | 20 | −0.975 | −0.535 | −0.390 | −0.626 | 1.219 | −0.184 | 0.770 | 0.487 | 1.187 | 41 | 11488605 |
| flunarizine | 9.88 | 0.920 | 1.573 | −0.053 | −0.212 | 0.803 | −1.689 | 0.996 | 0.933 | 0.933 | 43 | 11467460 |
| flunarizine | 20 | 0.130 | 0.610 | −1.669 | 0.243 | 1.721 | −0.063 | −0.270 | −0.010 | −0.740 | 43 | 11489198 |
| glipizide | 8.98 | 1.340 | 0.139 | −1.320 | −0.409 | 0.177 | 0.287 | −0.192 | −0.053 | −0.480 | 72 | 11467279 |
| glibenclamide | 8.1 | 0.655 | 0.370 | −0.746 | −0.168 | 0.944 | −0.277 | 0.624 | 0.444 | 0.885 | 74 | 11467464 |
| glyburide | 20 | 0.036 | −0.140 | −0.865 | −0.667 | −0.202 | 0.034 | −0.452 | −0.294 | −0.648 | 74 | 11489632 |
| loperamide | 8.38 | 0.655 | −0.924 | −0.753 | −0.252 | −0.237 | 0.014 | −1.005 | −0.954 | −0.954 | 80 | 11467292 |
| loperamide | 20 | −0.495 | −1.042 | −2.178 | −0.977 | 0.514 | −0.157 | 0.035 | 0.085 | −0.036 | 80 | 11489554 |
| minoxidil | 19.12 | 0.377 | −0.208 | −0.316 | −0.108 | 0.557 | 0.324 | 2.205 | 2.377 | 1.374 | 82 | 11467168 |
| minoxidil | 20 | 0.011 | 0.333 | −1.328 | −0.209 | 0.599 | 0.224 | −0.177 | −0.186 | −0.060 | 82 | 11488869 |
| nicardipine | 8.34 | −0.025 | −0.278 | −0.660 | −0.112 | 2.177 | −0.164 | −0.406 | −0.487 | −0.145 | 86 | 11467531 |
| nicardipine | 20 | −0.049 | −1.601 | −1.826 | 0.396 | 0.528 | 0.154 | −0.241 | −0.238 | −0.193 | 86 | 11489231 |
| retinoic acid | 13.32 | −0.077 | 0.727 | −1.296 | 0.099 | −0.331 | −0.866 | 0.807 | 0.763 | 0.727 | 104 | 11467405 |
| tretinon | 20 | −0.349 | −1.203 | −1.643 | 0.517 | −1.184 | −0.225 | −0.660 | −0.694 | −0.506 | 104 | 11489799 |
| nifedipine | 11.54 | −0.782 | −0.138 | −1.933 | 0.698 | 0.731 | 0.434 | −0.290 | −0.422 | −0.012 | 110 | 11467211 |
| nifedipine | 20 | 0.731 | −0.073 | −1.714 | 0.580 | 0.467 | 0.202 | 0.053 | 0.084 | 0.065 | 110 | 11488874 |
| niflumic acid | 14.18 | −0.133 | 0.819 | −1.178 | −0.488 | 1.521 | 0.459 | 0.215 | 0.305 | −0.015 | 112 | 11467403 |
| niflumic acid | 20 | −0.997 | 0.006 | −0.709 | −0.522 | 1.253 | 0.836 | −0.628 | −0.814 | −0.143 | 112 | 11488610 |
| nimodipine | 9.56 | −1.133 | 0.613 | 0.071 | 0.075 | 0.329 | 0.124 | −1.405 | −1.473 | −1.010 | 115 | 11468066 |
| nimodipine | 20 | 0.852 | 0.363 | −1.205 | 0.105 | 0.608 | −0.646 | −1.093 | −0.908 | −1.258 | 115 | 11489378 |
| nitrendipine | 11.1 | −0.538 | 0.016 | −0.400 | −0.513 | 0.085 | −0.558 | 0.626 | 0.562 | 0.625 | 117 | 11468064 |
| nitrendipine | 20 | −0.219 | −0.492 | −2.075 | −0.565 | 0.233 | −0.341 | −0.229 | −0.214 | −0.215 | 117 | 11489381 |
| 5-nitro-2-phenylpropylaminobenzoic acid | 20 | −0.939 | 0.261 | −0.786 | −0.937 | 0.872 | 0.412 | 0.038 | 0.089 | −0.075 | 121 | 11489293 |
| 3,3′-diindolylmethane | 20 | 1.350 | −0.064 | −0.873 | −0.513 | 1.667 | 0.644 | −0.544 | −0.389 | −0.711 | 122 | 11489527 |
| clofibrate | 20 | 0.797 | 0.355 | −0.842 | 0.089 | 0.576 | 0.336 | 0.000 | 0.030 | −0.080 | 142 | 11489025 |
| tetrandrine | 6.42 | −0.176 | −1.161 | −1.052 | 0.143 | 0.344 | −3.953 | 0.454 | 0.464 | 0.345 | 193 | 11467818 |
| tetrandrine | 20 | −2.453 | −5.953 | −4.728 | −3.304 | −1.379 | −0.378 | −0.806 | −0.814 | −0.676 | 193 | 11487841 |
| tolazamide | 12.84 | −0.354 | −1.359 | −0.851 | −0.617 | 0.262 | −0.182 | −0.146 | −0.071 | −0.266 | 196 | 11467702 |
| tolazamide | 20 | 1.405 | 0.628 | −0.644 | −0.095 | −0.036 | 0.193 | 0.033 | 0.035 | 0.025 | 196 | 11489265 |
| tolbutamide | 14.8 | −0.048 | −0.401 | −0.343 | −0.734 | 1.202 | −1.275 | −0.382 | −0.449 | −0.210 | 198 | 11467338 |
| tolbutamide | 20 | 0.711 | 0.224 | −1.174 | 1.011 | −1.033 | 0.113 | 1.206 | 1.186 | 1.075 | 198 | 11489026 |
| alprostadil | 11.28 | −0.155 | −0.499 | −0.185 | −0.436 | 0.420 | −0.260 | 1.028 | 0.889 | 1.101 | 220 | 11468166 |
| propidium iodide | 9.64 | 0.395 | 0.834 | −0.491 | −1.215 | 0.001 | 0.460 | −0.233 | 0.355 | −1.402 | 244 | 11467940 |
| phorbol myristate acetate | 20 | 0.649 | 0.248 | 1.936 | −0.464 | 0.933 | 0.530 | 0.816 | 1.109 | 0.086 | 290 | 11489727 |
| anisomycin | 20 | −3.560 | −1.993 | −4.207 | −0.220 | −2.145 | −2.269 | 1.482 | 2.276 | −0.371 | 336 | 11488448 |
| aminopyridine | 20 | −0.911 | 0.747 | −1.395 | −0.568 | 0.860 | 0.235 | −0.074 | 0.082 | −0.380 | 338 | 11489229 |
| piroxicam | 12.08 | 0.909 | −0.108 | −1.336 | −0.791 | 0.629 | 0.348 | −0.643 | −0.715 | −0.429 | 347 | 11467359 |
| piroxicam | 20 | −0.690 | 1.203 | −0.477 | −0.457 | 0.602 | 0.154 | −0.280 | −0.357 | −0.079 | 347 | 11489103 |
| terazosin | 10.32 | −0.367 | −0.241 | −0.654 | −0.753 | 0.330 | −0.258 | −0.111 | −0.106 | −0.115 | 349 | 11467899 |
| prazosin | 10.44 | −0.278 | −0.086 | −0.811 | 0.111 | 0.999 | 0.767 | −0.027 | −0.202 | 0.322 | 349 | 11468095 |
| prazosin | 20 | 0.400 | 0.937 | −0.518 | 0.465 | 0.443 | −0.394 | −0.014 | 0.021 | −0.084 | 349 | 11489105 |
| propranolol | 15.42 | 0.518 | 0.232 | −0.027 | 0.082 | 0.251 | 1.155 | 1.437 | 1.181 | 1.677 | 351 | 11468100 |
| propranolol | 20 | 0.359 | 0.830 | −0.670 | −0.344 | 0.547 | −1.186 | −0.434 | −0.473 | −0.270 | 351 | 11489117 |
| propranolol | 20 | −0.524 | −2.413 | −1.919 | 0.120 | −0.597 | 0.401 | 0.862 | 0.874 | 0.708 | 351 | 11489515 |
| quercetin | 13.24 | 0.816 | 0.716 | −1.595 | −1.691 | 0.374 | −0.214 | −0.340 | −0.086 | −0.785 | 353 | 11467655 |
| quercetin | 20 | 0.686 | 0.361 | −0.928 | −1.240 | 0.615 | 0.377 | 0.140 | −0.102 | 0.546 | 353 | 11487875 |
| diltiazem | 9.64 | −0.495 | 0.024 | −2.102 | −1.238 | 0.103 | −0.201 | −1.187 | −1.266 | −0.849 | 355 | 11467282 |
| flecainide | 9.66 | −0.112 | 1.210 | −1.170 | −1.026 | 0.987 | 0.763 | −0.167 | 0.006 | −0.486 | 359 | 11467883 |
| apigenin | 14.8 | −0.523 | −0.268 | −1.068 | −1.417 | 0.883 | −1.047 | −0.396 | −0.501 | −0.117 | 360 | 11467562 |
| naringenin | 14.7 | 0.350 | 0.892 | −0.475 | −0.620 | 0.735 | 1.489 | 0.085 | 0.046 | 0.159 | 360 | 11467614 |
| apigenin | 20 | 0.387 | 1.876 | −0.297 | −1.203 | 1.595 | −0.380 | 0.485 | 0.396 | 0.612 | 360 | 11488244 |
| lidocaine | 17.06 | −1.121 | 0.033 | −0.512 | −0.982 | 0.948 | 0.357 | −0.087 | −0.302 | 0.323 | 362 | 11467198 |
| lidocaine | 20 | −0.795 | 0.646 | −0.546 | −0.204 | −0.007 | 0.074 | −0.274 | −0.371 | −0.016 | 362 | 11489159 |
| statil | 20 | −0.878 | 0.377 | −1.350 | −0.494 | 0.409 | −0.105 | −0.398 | −0.272 | −0.576 | 366 | 11489283 |
| tamoxifen | 10.76 | 0.732 | −0.361 | 0.087 | −0.378 | 0.428 | 0.293 | 0.383 | 0.170 | 0.702 | 368 | 11467294 |
| tamoxifen | 20 | 0.201 | 0.489 | −0.180 | 0.462 | 0.951 | −0.227 | −0.628 | −0.461 | −0.869 | 368 | 11488705 |
| thalidomide | 15.5 | −0.577 | 1.142 | −1.400 | −0.697 | 0.788 | −0.213 | −1.574 | −1.692 | −1.073 | 370 | 11467340 |
| thalidomide | 20 | −1.042 | 0.264 | −0.660 | −0.645 | 0.263 | 0.020 | −0.520 | −0.608 | −0.249 | 370 | 11488523 |
| N-aminohexyl-5-chloro-1- | 20 | −1.182 | −0.809 | −1.143 | 0.009 | 0.879 | −0.695 | −0.390 | −0.463 | −0.157 | 382 | 11489385 |
| napthalenesulfonamide | | | | | | | | | | | | |
| camptothecin | 11.48 | −1.842 | −1.251 | −3.190 | 1.630 | −0.859 | −0.840 | −0.365 | −0.580 | 0.103 | 383 | 11467348 |
| camptothecin | 20 | −2.098 | −0.243 | −2.979 | 1.140 | −1.628 | −2.069 | −1.184 | −1.166 | −1.027 | 383 | 11488719 |
| estradiol-17 beta | 14.68 | 0.327 | −0.194 | 0.103 | −0.308 | −0.074 | 0.327 | 0.056 | −0.032 | 0.221 | 386 | 11467589 |
| riluzole | 17.08 | 1.079 | 1.652 | −1.861 | −0.392 | 0.476 | −0.694 | 0.523 | 0.648 | 0.127 | 399 | 11467315 |
| riluzole | 20 | 0.036 | −0.088 | −1.114 | 0.041 | 0.461 | 1.599 | 0.506 | 0.430 | 0.607 | 399 | 11488366 |
| aristolochic acid | 20 | −0.635 | 0.691 | −1.200 | −0.216 | 0.460 | 0.053 | −0.519 | −0.268 | −0.943 | 401 | 11488638 |
| bumetanide | 10.98 | −0.653 | 0.214 | −1.517 | −0.822 | 0.659 | 0.074 | −0.294 | −0.142 | −0.557 | 404 | 11467424 |
| bumetanide | 20 | −0.231 | 1.035 | −0.217 | 1.098 | 1.190 | 0.148 | 0.205 | 0.245 | 0.153 | 404 | 11488866 |
| clozapine | 12.24 | 1.080 | −0.716 | 0.190 | −0.197 | 0.948 | −0.153 | 0.781 | 0.861 | 0.463 | 417 | 11467498 |
| clozapine | 20 | −0.701 | 0.585 | −0.595 | −0.309 | 0.824 | 0.242 | 0.523 | 0.744 | −0.046 | 417 | 11488735 |
| adenosine | 20 | −0.902 | 0.787 | −1.853 | −0.736 | 1.310 | −0.339 | −1.258 | −1.131 | −1.202 | 418 | 11489073 |
| 3-methyl-1-phenyl-2-pyrazolin-5-one | 20 | −0.103 | 0.165 | −1.969 | −1.040 | 0.249 | 0.353 | −0.331 | −0.202 | −0.528 | 419 | 11489390 |
| juglone | 20 | −1.067 | −0.610 | −1.295 | −1.636 | 1.141 | 0.266 | 0.050 | 0.116 | −0.102 | 422 | 11488594 |
| genistein | 20 | −0.013 | 0.514 | −0.560 | −0.400 | 0.761 | 0.295 | 0.211 | 0.129 | 0.389 | 425 | 11488454 |
| serotonin | 22.7 | 1.124 | 1.590 | −0.263 | −0.305 | −1.319 | 1.163 | −1.359 | −0.945 | −1.934 | 429 | 11467629 |
| hydroxyurea | 20 | 0.084 | −0.221 | −0.728 | −0.952 | 0.158 | 0.147 | −0.895 | −1.022 | −0.523 | 430 | 11487880 |
| 3-isobutyl-1-methylxanthine | 20 | −0.534 | 0.030 | −1.643 | −0.967 | 0.472 | −0.063 | 0.176 | 0.125 | 0.288 | 435 | 11489521 |
| chlorpromazine | 12.54 | −0.895 | −1.056 | −1.081 | −0.711 | 0.894 | −0.093 | −0.308 | −0.273 | −0.370 | 436 | 11467212 |
| chlorpromazine | 20 | −0.936 | −0.174 | −1.529 | −1.057 | 0.542 | −0.615 | 0.070 | −0.022 | 0.308 | 436 | 11488972 |
| trifluoperazine | 9.82 | −0.169 | 0.516 | −0.246 | 0.745 | −0.087 | −0.965 | −0.760 | −0.656 | −0.825 | 437 | 11467461 |
| trifluoperazine | 20 | 0.645 | 0.525 | −0.537 | 1.071 | 1.056 | 0.186 | −0.557 | −0.825 | 0.074 | 437 | 11488644 |
| nocodazole | 13.28 | −0.069 | −0.969 | −0.751 | 0.358 | −1.099 | −2.032 | 1.312 | 1.429 | 0.763 | 440 | 11467248 |
| 3-aminobenzamide | 20 | −0.783 | 0.672 | −0.977 | −0.836 | 1.118 | 0.211 | −0.293 | −0.261 | −0.299 | 445 | 11489393 |
| capsaicin | 13.1 | −0.028 | −0.974 | −0.454 | −0.045 | 0.809 | 0.064 | −0.724 | −0.737 | −0.543 | 446 | 11468027 |
| E-capsaicin | 20 | −0.622 | 0.472 | −0.633 | −0.489 | 1.016 | 0.316 | 0.497 | 0.422 | 0.523 | 446 | 11488586 |
| clonidine | 17.38 | −0.791 | 2.923 | −0.250 | −0.056 | 0.584 | 0.018 | 0.188 | 0.256 | 0.018 | 448 | 11467396 |
| clonidine | 20 | −0.836 | 0.357 | −0.503 | −0.557 | 0.855 | 1.038 | −0.315 | −0.218 | −0.364 | 448 | 11489003 |
| menadione | 23.24 | −5.459 | −8.388 | −6.085 | −3.741 | −3.391 | −5.555 | −1.702 | −3.398 | 2.126 | 449 | 11467607 |
| menadione | 20 | −5.205 | −8.345 | −6.037 | −3.654 | −3.319 | −5.638 | −3.620 | −4.120 | −1.870 | 449 | 11489010 |
| corynanthine | 11.28 | −0.187 | 0.393 | −1.425 | −1.048 | 0.909 | 0.842 | 0.320 | 0.300 | 0.280 | 450 | 11467726 |
| caffeine | 20 | −0.404 | −0.397 | −0.358 | −0.718 | 0.914 | 0.292 | −0.120 | 0.084 | −0.451 | 451 | 11489077 |
| methotrexate | 8.8 | −0.299 | 0.543 | −1.961 | −0.905 | 0.231 | −0.098 | −0.928 | −1.019 | −0.608 | 464 | 11467283 |
| methotrexate | 20 | −1.003 | 1.034 | −2.307 | −1.098 | 0.599 | −0.453 | 0.749 | 0.780 | 0.616 | 464 | 11488893 |
| histamine | 20 | −0.723 | −0.369 | −1.327 | −0.900 | 0.225 | 0.016 | 0.399 | 0.518 | 0.074 | 465 | 11488481 |
| phenylbutyric acid | 20 | −1.328 | 0.127 | −0.797 | 0.217 | 0.153 | −0.651 | −0.799 | −0.643 | −0.913 | 470 | 11489614 |
| valproate | 20 | −0.340 | 0.535 | −0.680 | −0.784 | 0.809 | 0.530 | 0.517 | 0.657 | 0.117 | 471 | 11488762 |
| daidzein | 20 | −0.071 | −0.900 | −1.113 | −0.893 | 0.224 | 0.361 | −0.561 | −0.558 | −0.507 | 592 | 11487869 |
| ellagic acid | 20 | −0.435 | 0.994 | −1.503 | −2.499 | 0.347 | 0.534 | −0.651 | −0.623 | −0.605 | 598 | 11488721 |
| emodin | 20 | 0.003 | −0.079 | −1.274 | −3.895 | 1.558 | 0.021 | −0.763 | −0.781 | −0.592 | 599 | 11488711 |
| phloretin | 20 | 0.520 | 0.713 | −0.645 | −1.675 | 0.245 | 0.383 | −0.559 | −0.541 | −0.500 | 647 | 11488497 |
| purpurogallin | 20 | 0.034 | 1.972 | −1.771 | −1.670 | −0.374 | 0.635 | −0.482 | −0.534 | −0.237 | 653 | 11488398 |
| baclofen | 18.72 | −0.598 | 0.492 | −0.615 | −0.413 | 0.411 | 0.247 | −0.427 | −0.363 | −0.517 | 678 | 11467233 |
| baclofen | 20 | 0.433 | 0.099 | −0.221 | −0.022 | 0.824 | 1.066 | −0.272 | −0.435 | 0.055 | 678 | 11487908 |
| acetarsol | 20 | 0.084 | −0.893 | −1.621 | −0.436 | 0.582 | 0.117 | −0.156 | −0.262 | 0.036 | 679 | 11487920 |
| promethazine | 14.06 | 0.035 | 1.538 | 0.239 | 0.684 | 0.803 | −0.991 | 0.327 | 0.225 | 0.475 | 681 | 11468036 |
| promethazine | 20 | −0.409 | −0.487 | −0.777 | 0.115 | −0.006 | 0.128 | −0.894 | −0.905 | −0.712 | 681 | 11488656 |
| cortisone | 20 | 0.351 | 0.230 | −1.039 | −0.669 | 0.568 | −0.256 | −0.564 | −0.594 | −0.318 | 682 | 11488952 |
| metronidazole | 23.38 | 0.496 | 1.379 | −1.048 | −0.070 | −0.940 | 0.700 | 0.888 | 1.173 | 0.084 | 683 | 11467229 |
| metronidazole | 20 | 1.069 | 2.214 | −0.592 | 0.062 | −0.176 | 0.529 | −0.512 | −0.512 | −0.427 | 683 | 11488699 |
| erythromycin estolate | 20 | −0.315 | 0.361 | −0.837 | −0.707 | 0.889 | 0.022 | −0.138 | −0.261 | 0.144 | 684 | 11489251 |
| kinetin | 20 | 0.213 | 1.796 | −0.520 | 0.906 | 0.764 | 0.013 | −0.124 | −0.047 | −0.250 | 686 | 11489180 |
| reserpine | 6.58 | 0.678 | 0.494 | −0.706 | −0.616 | 1.962 | 0.257 | 0.355 | 0.230 | 0.549 | 687 | 11468023 |
| cefazolin | 8.8 | 0.064 | 0.611 | −1.945 | −0.676 | 1.124 | −0.902 | −1.152 | −1.260 | −0.711 | 689 | 11467884 |
| cefazolin | 20 | 0.362 | 0.428 | −1.291 | −0.045 | 0.543 | 0.674 | 0.036 | 0.110 | −0.043 | 689 | 11488956 |
| alprenolol | 16.04 | 0.933 | 1.466 | 0.118 | 0.360 | 0.664 | 0.748 | 0.137 | 0.236 | −0.107 | 690 | 11467398 |
| alprenolol | 20 | 0.025 | 0.158 | −1.125 | −0.727 | 0.473 | −0.043 | −1.425 | −1.198 | −1.561 | 690 | 11489630 |
| azlocillin | 8.66 | 0.709 | 0.830 | −1.241 | −0.741 | 1.825 | 0.178 | −0.349 | −0.030 | −0.927 | 691 | 11467969 |
| azlocillin | 20 | 1.605 | 1.488 | −0.900 | 0.242 | −0.785 | 1.060 | −1.102 | −1.013 | −1.072 | 691 | 11489338 |
| acetazolamide | 18 | −0.481 | 3.862 | −0.192 | 0.513 | 0.338 | 0.698 | −0.621 | −0.495 | −0.801 | 692 | 11467151 |
| acetazolamide | 20 | −0.196 | −0.106 | −0.676 | 1.204 | −0.322 | 0.613 | 0.025 | −0.283 | 0.588 | 692 | 11487898 |
| tilorone | 20 | −3.332 | −3.413 | 0.202 | 0.082 | −1.170 | −1.071 | −0.585 | −0.515 | −0.574 | 693 | 11489558 |
| fluorometholone | 20 | −1.056 | 0.060 | −1.367 | −0.308 | 0.548 | −0.466 | 0.146 | 0.294 | −0.097 | 694 | 11489082 |
| semustine | 20 | −0.152 | 0.546 | −1.124 | −0.874 | 0.493 | 0.374 | −1.153 | −0.849 | −1.568 | 695 | 11488727 |
| anthralin | 20 | 0.064 | −1.114 | −1.260 | −2.161 | 0.943 | 0.348 | −0.848 | −0.627 | −1.187 | 696 | 11487927 |
| diprophylline | 15.74 | −0.984 | −0.329 | −1.657 | −0.686 | 1.868 | 0.118 | −0.188 | −0.208 | −0.160 | 697 | 11467181 |
| dyphylline | 20 | 0.622 | 0.530 | 0.201 | 1.225 | 0.735 | 0.010 | 0.844 | 0.615 | 1.085 | 697 | 11487906 |
| fenbufen | 15.74 | −0.558 | −0.004 | −1.609 | −0.707 | 0.460 | 0.051 | 0.578 | 0.553 | 0.481 | 699 | 11467366 |
| fenbufen | 20 | −0.148 | −0.649 | −0.708 | −0.574 | 0.895 | −0.284 | 0.001 | −0.028 | 0.059 | 699 | 11489205 |
| homatropine | 20 | −0.393 | 0.315 | −0.970 | −0.642 | 0.643 | −0.164 | −1.198 | −0.968 | −1.364 | 700 | 11488795 |
| ambroxol | 10.58 | −0.619 | 0.728 | −0.752 | −1.193 | 2.504 | −0.108 | 0.145 | 0.128 | 0.151 | 701 | 11467514 |
| ambroxol | 20 | 1.395 | 0.085 | 0.917 | 0.349 | 0.584 | −0.149 | −0.060 | −0.041 | −0.086 | 701 | 11489334 |
| hydroxyprogesterone | 20 | −0.409 | −1.768 | −0.554 | 1.288 | −1.178 | −2.065 | 0.042 | 0.308 | −0.466 | 702 | 11488346 |
| salicin | 20 | −1.626 | −0.052 | −2.227 | −0.766 | 1.012 | 0.336 | 0.676 | 0.712 | 0.461 | 703 | 11488572 |
| gentian violet | 20 | −3.137 | −5.276 | −5.314 | −3.944 | −2.488 | −3.653 | −2.735 | −1.196 | −5.260 | 704 | 11488904 |
| benfluorex | 11.38 | −0.271 | 0.873 | −0.780 | −0.807 | 2.384 | 0.309 | −1.547 | −1.082 | −2.217 | 705 | 11467515 |
| benfluorex | 20 | 0.509 | 0.548 | −1.221 | −0.621 | 0.943 | −0.162 | −0.641 | −0.466 | −0.790 | 705 | 11489033 |
| sulfaquinoxaline | 13.32 | 0.879 | 0.512 | −0.938 | −0.564 | −0.447 | −0.233 | −0.502 | −0.435 | −0.553 | 706 | 11467879 |
| sulfaquinoxaline | 20 | −0.211 | 0.118 | −1.764 | −0.469 | 0.698 | 0.649 | −0.182 | −0.272 | 0.104 | 706 | 11488802 |
| digitoxin | 20 | −0.052 | 0.694 | −1.108 | 0.910 | 0.919 | −0.679 | −0.125 | −0.362 | 0.313 | 707 | 11487886 |
| astemizole | 8.72 | −3.664 | −4.284 | −5.349 | −0.384 | −1.650 | −4.866 | 0.336 | 0.208 | 0.494 | 709 | 11467284 |
| astemizole | 20 | −5.634 | −8.294 | −6.684 | −4.127 | −3.597 | −3.496 | −3.310 | −3.860 | −1.480 | 709 | 11489548 |
| cephalosporin C | 20 | −1.140 | 0.930 | −2.039 | −0.512 | −0.483 | 0.357 | −0.894 | −0.988 | −0.484 | 710 | 11488331 |
| resorcinol | 20 | −0.117 | −0.372 | −0.009 | −0.154 | 1.313 | −0.863 | −0.102 | −0.004 | −0.276 | 711 | 11489126 |
| cephapirin | 9.44 | −0.570 | −0.536 | −2.017 | −0.864 | 0.544 | −0.120 | −0.168 | −0.135 | −0.202 | 712 | 11467999 |
| cephapirin | 20 | −0.201 | −0.089 | −1.765 | −0.933 | 0.767 | 0.445 | 0.471 | 0.365 | 0.541 | 712 | 11487919 |
| mebeverine | 9.32 | −1.262 | −0.521 | −0.344 | 0.316 | 1.404 | −0.073 | 0.173 | 0.048 | 0.393 | 714 | 11467458 |
| mebeverine | 20 | −1.152 | 0.368 | −1.358 | −0.783 | 0.761 | −1.401 | −0.917 | −0.749 | −1.071 | 714 | 11489220 |
| khellin | 15.38 | −0.206 | −0.004 | −1.317 | −0.176 | −0.017 | −0.002 | −0.889 | −0.890 | −0.748 | 715 | 11467239 |
| khellin | 20 | −0.967 | 0.407 | −2.053 | −0.473 | 1.119 | 0.584 | −1.451 | −1.491 | −1.058 | 715 | 11488409 |
| cyclobenzaprine | 14.52 | −0.881 | −0.183 | −1.981 | −0.736 | 0.031 | −2.311 | −1.238 | −1.087 | −1.303 | 716 | 11467593 |
| cyclobenzaprine | 20 | −1.284 | −3.850 | −3.640 | −0.570 | −2.292 | −0.625 | −0.371 | −0.554 | 0.075 | 716 | 11489350 |
| fosfosal | 18.34 | 0.253 | 0.528 | −1.655 | −0.997 | 1.703 | 0.254 | −0.831 | −0.750 | −0.842 | 717 | 11467963 |
| fosfosal | 20 | −0.460 | 1.564 | −1.240 | −0.757 | 0.469 | 0.647 | −0.488 | −0.394 | −0.585 | 717 | 11489274 |
| etofylline | 17.84 | 0.254 | 1.247 | −1.301 | −0.616 | 0.320 | −0.750 | 0.681 | 0.706 | 0.438 | 718 | 11467320 |
| 7-hydroxyethyltheophylline | 20 | −0.541 | −0.071 | −0.478 | −0.294 | 0.055 | 0.496 | 0.285 | 0.311 | 0.205 | 718 | 11489635 |
| pargyline | 25.12 | −0.056 | 0.850 | −1.666 | −0.243 | 1.283 | −0.646 | −0.600 | −0.523 | −0.689 | 719 | 11467331 |
| pargyline | 20 | −0.007 | 0.058 | −0.197 | −0.450 | 1.772 | 0.252 | 0.282 | 0.238 | 0.386 | 719 | 11488855 |
| fluorouracil | 20 | 0.778 | 1.481 | −1.998 | −0.089 | 0.169 | −0.531 | 1.643 | 1.502 | 1.554 | 720 | 11487892 |
| oleandomycin | 20 | −0.560 | 0.560 | −0.960 | −0.735 | 0.410 | −0.672 | −0.480 | −0.571 | −0.203 | 721 | 11488663 |
| probenecid | 14.02 | 0.196 | −0.134 | −1.052 | −0.526 | 0.706 | 0.495 | −0.414 | −0.312 | −0.534 | 722 | 11467690 |
| probenecid | 20 | −1.102 | 0.967 | −1.257 | −1.339 | −0.269 | 0.059 | −0.937 | −0.928 | −0.787 | 722 | 11489110 |
| atenolol | 20 | 0.708 | 1.023 | −0.547 | −0.798 | 0.508 | 0.403 | −0.609 | −0.304 | −1.106 | 723 | 11489227 |
| nalidixic acid | 17.22 | −0.474 | 0.071 | −1.669 | −0.686 | 0.845 | −0.710 | 0.547 | 0.728 | 0.034 | 724 | 11467335 |
| nalidixic acid | 20 | −0.157 | 0.957 | 0.019 | 0.278 | 1.064 | 0.412 | 0.797 | 0.692 | 0.861 | 724 | 11489176 |
| perillic acid | 20 | −1.102 | 0.038 | −1.117 | −0.470 | 0.937 | 0.117 | −0.366 | −0.329 | −0.376 | 725 | 11488744 |
| urethane | 20 | −0.741 | 0.192 | −0.621 | 0.019 | 1.356 | 0.472 | 0.359 | 0.370 | 0.242 | 726 | 11488725 |
| ethopropazine | 12.8 | −1.343 | −0.482 | −0.890 | −0.143 | 0.153 | 0.284 | −0.757 | −0.332 | −1.452 | 727 | 11467988 |
| ethopropazine | 20 | −1.421 | 0.713 | −0.393 | −0.956 | −0.292 | 0.259 | −0.392 | −0.269 | −0.493 | 727 | 11488800 |
| minaprine | 13.4 | −1.866 | 0.148 | −1.670 | −1.010 | 1.093 | −0.119 | 0.087 | 0.170 | −0.138 | 728 | 11467214 |
| minaprine | 20 | −0.227 | 0.186 | −2.047 | −1.072 | 1.120 | 0.920 | −0.719 | −0.790 | −0.438 | 728 | 11489223 |
| lactulose | 20 | −0.225 | 0.319 | −0.725 | −0.022 | 0.900 | −0.806 | −0.650 | −0.903 | 0.097 | 729 | 11488975 |
| thioridazine | 10.8 | −1.056 | 0.008 | −1.004 | −0.460 | 1.385 | −1.505 | 0.513 | 0.164 | 1.081 | 731 | 11467226 |
| thioridazine | 20 | −0.071 | −0.362 | −1.520 | −0.366 | −1.412 | −0.143 | −0.717 | −0.462 | −1.098 | 731 | 11489148 |
| 3,5-dinitrocatechol | 20 | −0.814 | −0.504 | −1.388 | −0.705 | 1.411 | 0.671 | −0.293 | −0.357 | −0.020 | 732 | 11488925 |
| memantine | 22.3 | 0.886 | 0.276 | 0.208 | 0.236 | 0.881 | −0.342 | −0.672 | −0.724 | −0.446 | 733 | 11468126 |
| memantine | 20 | −0.548 | 0.065 | −1.104 | −0.663 | 1.706 | 0.243 | −0.312 | 0.128 | −1.117 | 733 | 11489224 |
| metoclopramide | 13.34 | −1.023 | −0.701 | −1.155 | −0.701 | 1.422 | 0.037 | −0.296 | −0.541 | 0.222 | 734 | 11467357 |
| metoclopramide | 20 | −0.332 | 0.721 | −0.457 | 0.047 | 1.034 | 0.000 | −0.588 | −0.784 | −0.039 | 734 | 11489536 |
| isoniazid | 29.16 | 1.107 | 1.676 | −0.222 | 0.320 | −0.562 | 0.260 | −1.181 | −1.256 | −0.836 | 735 | 11467309 |
| isoniazid | 20 | −0.513 | −0.061 | −1.832 | −0.697 | 0.957 | 1.056 | 0.418 | 0.499 | 0.120 | 735 | 11487923 |
| mecysteine | 20 | −0.261 | 0.371 | −1.306 | −0.509 | 0.833 | 0.502 | 0.036 | 0.092 | −0.134 | 736 | 11487830 |
| tiabendazole | 19.88 | −0.786 | −0.171 | −1.873 | −1.093 | 0.578 | 0.746 | −0.840 | −0.902 | −0.549 | 737 | 11467672 |
| thiabendazole | 20 | 0.175 | −0.132 | −0.902 | −0.592 | −0.278 | −0.449 | −0.525 | −0.413 | −0.665 | 737 | 11489147 |
| acetanilide | 20 | −1.150 | 0.621 | −1.213 | −1.104 | 1.145 | 0.532 | 0.043 | 0.206 | −0.295 | 738 | 11489250 |
| glutathione | 20 | −0.137 | 0.643 | −0.267 | −0.067 | 0.732 | −0.913 | 0.406 | 0.459 | 0.215 | 740 | 11489316 |
| mephenesin | 21.96 | −0.082 | 0.246 | −0.134 | −0.680 | 1.161 | −0.031 | 0.222 | 0.394 | −0.219 | 741 | 11467326 |
| mephenesin | 20 | −1.140 | −0.555 | −1.677 | −0.506 | 0.945 | 0.922 | 0.154 | 0.349 | −0.273 | 741 | 11489234 |
| fusidic acid | 20 | −0.547 | 0.538 | −1.074 | −1.339 | 0.932 | 0.256 | 0.206 | 0.355 | −0.066 | 742 | 11489083 |
| terbutaline | 17.76 | −0.275 | 0.292 | −0.796 | −0.919 | 1.006 | −0.434 | −0.369 | −0.276 | −0.496 | 743 | 11467539 |
| terbutaline | 20 | −0.410 | 0.371 | −1.064 | −1.297 | 0.995 | 0.201 | 0.431 | 0.402 | 0.407 | 743 | 11489143 |
| paraxanthine | 20 | −0.737 | 0.570 | −1.990 | −0.216 | 0.456 | −0.027 | −0.685 | −0.604 | −0.680 | 744 | 11489549 |
| deferoxamine | 7.14 | 0.141 | 1.051 | −1.645 | −0.392 | −0.336 | 0.082 | 0.199 | 0.487 | −0.434 | 745 | 11467873 |
| deferoxamine | 20 | −1.272 | 0.653 | −2.241 | −0.456 | 0.098 | 1.697 | −0.915 | −0.825 | −0.848 | 745 | 11488971 |
| antazoline | 15.08 | 0.036 | 0.531 | −1.337 | −1.331 | 1.711 | −0.218 | 0.470 | 0.430 | 0.480 | 746 | 11467406 |
| antazoline | 20 | −0.129 | 0.474 | −0.981 | −0.920 | 0.992 | 0.721 | −0.264 | −0.238 | −0.188 | 746 | 11489075 |
| norfloxacin | 12.52 | 0.040 | −0.597 | −1.145 | −0.604 | 1.115 | −0.150 | −0.594 | −0.507 | −0.691 | 747 | 11467369 |
| norfloxacin | 20 | −0.669 | 0.116 | −1.055 | −0.901 | 0.285 | −0.477 | −0.259 | −0.300 | −0.047 | 747 | 11488833 |
| urea | 20 | 0.812 | 0.263 | 0.234 | 0.286 | −0.938 | 0.081 | 0.652 | 0.647 | 0.604 | 749 | 11489008 |
| streptomycin | 20 | −1.244 | 0.584 | −1.969 | −1.109 | 1.559 | −0.113 | 0.548 | 0.440 | 0.707 | 750 | 11488263 |
| sulfadimethoxine | 12.88 | 1.489 | 0.715 | 0.009 | −0.727 | −0.656 | 0.332 | 0.076 | 0.171 | −0.135 | 751 | 11467876 |
| sulfadimethoxine | 20 | −0.799 | −0.336 | −0.514 | −0.452 | 0.599 | 0.735 | −0.606 | −0.713 | −0.267 | 751 | 11489235 |
| flumequine | 15.32 | −1.610 | −0.507 | −1.735 | −0.579 | 0.909 | −0.675 | −0.652 | −0.927 | −0.009 | 752 | 11467352 |
| flumequine | 20 | −0.500 | 0.141 | 0.487 | 0.220 | −0.232 | −0.301 | −0.020 | 0.033 | −0.064 | 752 | 11489016 |
| sulfinpyrazone | 9.88 | −1.107 | 0.358 | −0.836 | −0.131 | 1.006 | 0.173 | 0.125 | 0.071 | 0.214 | 753 | 11467438 |
| sulfinpyrazone | 20 | −1.098 | −0.276 | −0.970 | −1.021 | 0.255 | −0.838 | −0.269 | −0.253 | −0.249 | 753 | 11489140 |
| trimipramine | 13.58 | 1.504 | 1.329 | −1.526 | −0.697 | 0.811 | −3.317 | −0.380 | −0.309 | −0.459 | 755 | 11467954 |
| trimipramine | 20 | −1.573 | −6.070 | −4.012 | 2.895 | −1.136 | 0.200 | −0.909 | −1.153 | −0.246 | 755 | 11489346 |
| hexylresorcinol | 20 | −0.106 | 0.408 | −0.510 | −0.732 | 0.175 | −0.245 | 0.194 | 0.077 | 0.483 | 756 | 11488805 |
| ciprofloxacin | 12.08 | −0.988 | 0.321 | −1.677 | −0.935 | 0.368 | −0.078 | −1.391 | −1.441 | −1.060 | 757 | 11467261 |
| ciprofloxacin | 20 | −1.222 | −0.033 | −1.635 | −0.738 | 0.564 | 0.186 | −0.635 | −0.648 | −0.483 | 757 | 11489383 |
| oxibendazole | 20 | −1.899 | −0.104 | −3.046 | −0.975 | −1.178 | −1.471 | 0.274 | 0.144 | 0.483 | 758 | 11489372 |
| cephalothin | 10.08 | 0.099 | −0.628 | −1.062 | −0.504 | 1.329 | 0.328 | 0.171 | 0.032 | 0.424 | 759 | 11467867 |
| cephalothin | 20 | 0.824 | −0.562 | −0.695 | −0.718 | 0.189 | 0.343 | 0.110 | 0.191 | −0.137 | 759 | 11487937 |
| (S)-(−)-cycloserine | 39.18 | −0.457 | −0.139 | 0.269 | 1.036 | −0.520 | 0.563 | −0.556 | −0.736 | −0.095 | 760 | 11468237 |
| cycloserine | 20 | −0.112 | 0.623 | −1.390 | 0.467 | 0.696 | −0.776 | 0.847 | 0.863 | 0.605 | 760 | 11487900 |
| methicillin | 20 | −0.246 | 0.669 | −0.759 | −0.497 | 0.706 | −0.111 | −0.080 | 0.024 | −0.309 | 762 | 11489781 |
| quinacrine | 10 | −0.579 | 0.313 | −1.080 | −1.194 | 2.788 | −3.708 | 1.007 | 0.703 | 1.432 | 763 | 11467466 |
| quinacrine | 20 | −6.002 | −8.309 | −1.910 | 0.428 | 0.865 | −0.684 | −2.890 | −2.650 | −2.810 | 763 | 11488704 |
| droperidol | 10.54 | −0.013 | −0.602 | −0.645 | 0.107 | 1.371 | 0.270 | 0.469 | 0.438 | 0.440 | 764 | 11467508 |
| droperidol | 20 | −0.670 | −0.627 | −1.157 | −0.131 | 0.927 | 0.161 | 0.524 | 0.622 | 0.222 | 764 | 11489202 |
| ethisterone | 20 | 0.086 | 0.439 | −1.214 | −1.355 | 0.240 | −0.080 | 0.590 | −0.570 | −0.500 | 766 | 11489353 |
| amygdalin | 20 | −0.928 | 0.720 | −1.739 | −0.290 | 1.161 | 0.613 | −0.243 | 0.013 | −0.747 | 767 | 11488720 |
| choline | 20 | −0.669 | 0.740 | −0.905 | −0.897 | 0.684 | 0.713 | −1.448 | −1.141 | −1.813 | 768 | 11489754 |
| bufexamac | 17.92 | −0.223 | 2.320 | −0.617 | −0.527 | −0.303 | 0.419 | −0.801 | −0.599 | −1.057 | 769 | 11467391 |
| bufexamac | 20 | 0.105 | 1.620 | −0.950 | −0.498 | 0.105 | 0.851 | 0.143 | 0.331 | −0.273 | 769 | 11489273 |
| nylidrin | 20 | −0.879 | 1.387 | 0.167 | −0.231 | 0.507 | 0.498 | −0.030 | −0.010 | −0.060 | 770 | 11488783 |
| ketotifen | 12.92 | 0.306 | −0.077 | −1.173 | −1.085 | 0.844 | −0.546 | −0.548 | −0.423 | −0.679 | 771 | 11467519 |
| ketotifen | 20 | −0.013 | 0.332 | 0.415 | −0.358 | 0.491 | 0.292 | 0.569 | 0.377 | 0.908 | 771 | 11489014 |
| piperidolate | 12.36 | −0.159 | 0.376 | −0.541 | −0.313 | 0.060 | −0.186 | −0.419 | −0.421 | −0.343 | 772 | 11468203 |
| piperidolate | 20 | −0.575 | −0.957 | −1.693 | −0.688 | 0.734 | 0.436 | −0.438 | −0.310 | −0.543 | 772 | 11488889 |
| econazole | 10.48 | 0.273 | −0.830 | −1.573 | −0.237 | 0.970 | −0.215 | 0.196 | 0.102 | 0.356 | 773 | 11467452 |
| econazole | 20 | 0.137 | −1.886 | −1.114 | 0.948 | 1.668 | 0.068 | 0.496 | 0.341 | 0.725 | 773 | 11489255 |
| aminohydroxybutyric acid | 20 | 0.634 | 0.467 | −0.889 | −0.073 | 0.067 | 0.084 | −0.127 | −0.072 | −0.138 | 775 | 11488945 |
| hydralazine | 24.98 | 0.572 | 0.791 | −0.465 | −0.591 | 0.325 | 0.204 | 0.181 | 0.315 | −0.168 | 776 | 11467317 |
| hydralazine | 20 | 0.882 | 0.692 | −0.830 | 0.069 | 0.162 | 0.687 | −0.497 | −0.379 | −0.579 | 776 | 11488785 |
| naringenin | 20 | −1.071 | 1.514 | −0.626 | 0.510 | 0.248 | 1.008 | −0.538 | −0.248 | −0.986 | 777 | 11488141 |
| iodoquinol | 20 | 0.242 | −0.055 | −0.120 | 2.621 | −0.493 | 0.912 | 0.116 | 0.033 | 0.331 | 778 | 11488857 |
| procaine | 16.92 | 0.539 | −0.246 | −1.237 | −0.685 | 0.875 | 0.260 | 0.441 | 0.516 | 0.150 | 779 | 11467189 |
| procaine | 20 | −0.848 | 0.642 | −0.685 | −0.169 | 0.116 | 0.215 | −0.322 | −0.407 | −0.090 | 779 | 11489112 |
| iproniazid | 22.32 | −0.416 | 0.691 | −0.696 | −0.958 | 0.753 | 0.472 | 0.206 | 0.355 | −0.179 | 780 | 11467324 |
| iproniazid | 20 | −1.496 | 0.708 | −0.643 | −0.831 | 1.440 | 0.597 | 0.755 | 0.484 | 1.212 | 780 | 11488284 |
| flunisolide | 20 | −0.717 | 0.018 | 0.186 | 0.076 | 0.363 | −0.257 | −0.472 | −0.790 | 0.272 | 782 | 11489256 |
| nicergoline | 8.26 | −0.633 | 0.977 | −1.186 | −0.869 | 0.630 | −0.858 | −0.438 | −0.438 | −0.385 | 783 | 11467295 |
| nicergoline | 20 | −0.876 | 0.405 | −1.563 | −0.832 | 0.491 | −0.486 | −0.498 | −0.405 | −0.582 | 783 | 11489230 |
| 5-azacytidine | 20 | −2.288 | 0.499 | −1.818 | −1.175 | 0.540 | −0.923 | 0.484 | 0.340 | 0.678 | 784 | 11488602 |
| pirenzepine | 11.38 | −0.696 | −0.699 | −0.776 | −0.242 | 0.363 | −0.017 | −2.019 | −2.150 | −1.402 | 786 | 11467277 |
| pirenzepine | 20 | −1.170 | 0.649 | −0.875 | −0.784 | 1.438 | −0.596 | −0.188 | −0.029 | −0.476 | 786 | 11489233 |
| homatropine | 20 | −0.068 | 1.047 | −0.917 | −0.524 | 0.072 | 0.399 | −0.397 | −0.320 | −0.425 | 789 | 11488360 |
| 1r,9s-hydrastine | 20 | −0.738 | 0.964 | −1.927 | −0.956 | 0.256 | 0.103 | −0.069 | 0.047 | −0.209 | 790 | 11488812 |
| quinine | 20 | 0.079 | 0.789 | −0.450 | 0.156 | 0.836 | −1.375 | −0.473 | −0.528 | −0.260 | 792 | 11489124 |
| amrinone | 21.36 | −0.418 | −0.009 | 0.106 | −0.180 | 0.107 | −0.100 | −0.124 | −0.147 | −0.028 | 794 | 11467948 |
| amrinone | 20 | −0.109 | 0.092 | −0.071 | −0.557 | 0.982 | −0.579 | −0.286 | −0.409 | −0.016 | 794 | 11489796 |
| spectinomycin | 12.04 | 0.964 | 0.661 | −1.288 | −0.344 | 0.096 | 0.125 | −0.503 | −0.293 | −0.829 | 795 | 11467952 |
| spectinomycin | 20 | −1.189 | 1.254 | −1.506 | −0.674 | 0.083 | 1.197 | −0.353 | −0.397 | −0.195 | 795 | 11489131 |
| gemfibrozil | 15.98 | −0.530 | 0.316 | −1.293 | −0.383 | 0.448 | 0.459 | −0.917 | −0.816 | −0.984 | 796 | 11467362 |
| gemfibrozil | 20 | −0.985 | 0.063 | −1.395 | −0.658 | 0.661 | 0.164 | −0.225 | −0.284 | 0.008 | 796 | 11488913 |
| monensin | 20 | −0.176 | −3.394 | −2.104 | 3.603 | −2.989 | −1.704 | −0.983 | 0.293 | −3.366 | 797 | 11489325 |
| exalamide | 20 | −0.103 | −0.629 | −0.465 | −0.028 | 0.385 | −0.362 | −0.229 | −0.152 | −0.369 | 798 | 11488685 |
| sulfamethizole | 14.8 | −0.521 | 0.497 | −1.733 | −0.450 | 0.468 | 0.322 | −0.326 | −0.154 | −0.628 | 799 | 11467890 |
| sulfamethizole | 20 | −0.244 | 0.866 | −0.483 | −0.745 | 0.025 | 0.644 | 0.062 | 0.096 | −0.029 | 799 | 11489138 |
| methyldopa | 18.94 | 0.286 | 0.504 | −1.421 | −1.719 | 0.203 | −1.204 | 0.836 | 0.962 | 0.404 | 800 | 11467474 |
| methyldopa | 20 | −0.286 | 1.115 | −0.784 | −0.842 | −0.145 | 0.755 | −0.020 | −0.084 | 0.203 | 800 | 11488884 |
| chlorprothixene | 20 | −0.476 | 0.273 | −0.824 | −0.650 | 0.495 | −0.305 | −0.605 | −0.636 | −0.439 | 801 | 11488673 |
| quinalizarin | 20 | 0.583 | 1.276 | −0.668 | −2.511 | 0.674 | 0.935 | −0.559 | −0.517 | −0.543 | 802 | 11489178 |
| ethionamide | 24.06 | −1.076 | −0.800 | −1.292 | −1.055 | 1.108 | 0.211 | −0.465 | −0.236 | −0.839 | 803 | 11467674 |
| ethionamide | 20 | −1.333 | −0.464 | −1.757 | −0.677 | 0.067 | −0.472 | −0.192 | −0.112 | −0.241 | 803 | 11488810 |
| mycophenolic acid | 12.48 | 0.030 | −1.446 | −1.697 | 0.026 | −0.251 | 0.220 | 0.100 | 0.062 | 0.155 | 804 | 11467704 |
| mycophenolic acid | 20 | −0.335 | −1.173 | −1.793 | 0.210 | −0.854 | −0.263 | −0.215 | −0.297 | −0.019 | 804 | 11488708 |
| etodolac | 13.92 | −0.733 | −0.841 | −0.715 | 0.319 | 0.379 | 0.172 | −0.440 | −0.496 | −0.297 | 805 | 11467379 |
| etodolac | 20 | −0.369 | −0.632 | −1.397 | −0.661 | 1.194 | 0.212 | 0.014 | 0.063 | −0.094 | 805 | 11489203 |
| niacin | 32.5 | −1.128 | 2.434 | 0.087 | 0.742 | −0.544 | 0.736 | −0.220 | 0.000 | −0.660 | 806 | 11468029 |
| nipecotic acid | 30.96 | −0.273 | 0.241 | 0.512 | 0.606 | 0.591 | −0.082 | 1.014 | 0.734 | 1.386 | 806 | 11468098 |
| niacin | 20 | 0.028 | −0.274 | −1.746 | 0.757 | 0.593 | 1.132 | −0.404 | −0.363 | −0.467 | 806 | 11487822 |
| nipecotic acid | 20 | −0.551 | 0.735 | −0.516 | −0.580 | 0.362 | −1.276 | −0.704 | −0.455 | −0.995 | 806 | 11489000 |
| amprolium | 16.44 | −0.583 | 1.227 | −0.304 | 0.135 | 1.134 | 0.126 | 0.136 | 0.195 | −0.055 | 807 | 11467156 |
| amprolium | 20 | 0.292 | 0.035 | −1.124 | −1.025 | 0.560 | 1.268 | 0.243 | 0.205 | 0.210 | 807 | 11487938 |
| nortriptyline | 15.18 | −0.668 | 0.209 | −2.020 | −0.399 | −0.159 | 0.157 | −0.292 | −0.233 | −0.354 | 809 | 11467402 |
| nortriptyline | 20 | −1.564 | 1.097 | −1.905 | −1.277 | 0.461 | 0.136 | 0.542 | 0.545 | 0.506 | 809 | 11488813 |
| antimycin A | 20 | −0.971 | −0.604 | −1.390 | 0.520 | 0.400 | −0.791 | −1.380 | −1.319 | −1.168 | 810 | 11488903 |
| pregnenolone | 20 | −0.360 | 0.445 | 1.358 | 0.286 | 0.338 | 0.971 | −1.061 | −0.586 | −1.854 | 811 | 11488758 |
| griseofulvin | 20 | 0.008 | −2.024 | −1.919 | 0.065 | −1.037 | −1.343 | 0.341 | 0.068 | 0.782 | 812 | 11488029 |
| estradiol diacetate | 20 | −0.748 | 0.784 | −0.624 | −0.924 | 1.404 | 0.396 | 0.379 | 0.457 | 0.148 | 813 | 11489253 |
| miconazole | 9.62 | −1.008 | −0.397 | −1.491 | −0.401 | 1.628 | 0.802 | −0.955 | −1.052 | −0.619 | 814 | 11467215 |
| miconazole | 20 | −0.134 | 0.051 | −0.885 | 0.765 | 1.933 | −0.524 | 0.078 | 0.136 | 0.013 | 814 | 11488864 |
| DEET | 20 | −0.046 | −0.064 | −1.052 | −0.574 | 0.608 | 0.065 | −0.830 | −0.631 | −1.009 | 815 | 11488888 |
| xylometazoline | 16.36 | −0.348 | −0.122 | −1.163 | −0.584 | 0.378 | 1.079 | −0.694 | −0.650 | −0.696 | 816 | 11467371 |
| xylometazoline | 20 | −0.870 | 0.050 | −0.710 | −1.094 | 0.720 | −0.231 | −0.478 | −0.223 | −0.915 | 816 | 11488761 |
| pyrithyldione | 23.92 | 0.815 | 2.084 | −0.650 | −0.254 | 0.035 | −0.828 | −0.724 | −0.504 | −1.035 | 818 | 11467951 |
| pyrithyldione | 20 | −0.767 | −0.173 | 0.262 | 0.351 | −0.612 | 0.514 | −0.245 | −0.531 | 0.394 | 818 | 11489336 |
| dicyclomine | 12.92 | −0.420 | −0.599 | −0.626 | −1.142 | 2.249 | −0.146 | 0.074 | −0.111 | 0.391 | 819 | 11467196 |
| dicyclomine | 20 | 0.195 | 1.104 | −0.650 | 0.053 | 1.345 | −0.506 | −0.098 | −0.166 | 0.106 | 819 | 11488406 |
| cloxyquin | 20 | 0.366 | −0.163 | −0.651 | 0.049 | −0.294 | 0.920 | −1.054 | −1.128 | −0.618 | 820 | 11488947 |
| saccharin | 20 | −0.178 | 0.908 | −0.357 | −0.895 | 0.429 | 0.336 | 0.192 | 0.283 | −0.035 | 821 | 11489248 |
| neostigmine | 17.92 | 0.205 | −0.066 | 0.641 | −0.516 | 0.904 | 0.368 | −0.244 | −0.340 | 0.007 | 822 | 11467616 |
| neostigmine | 20 | −0.604 | 0.055 | 1.344 | −0.614 | 1.299 | −0.193 | −3.767 | −3.541 | −3.446 | 822 | 11489094 |
| vincamine | 11.28 | −0.087 | 0.840 | −0.748 | −0.828 | 0.777 | −0.176 | 0.283 | 0.204 | 0.410 | 824 | 11467784 |
| vincamine | 20 | −0.819 | −0.619 | −0.445 | −0.717 | 0.244 | 0.118 | 0.003 | 0.039 | −0.073 | 824 | 11489154 |
| carbidopa | 20 | −2.898 | −0.867 | −2.142 | −2.029 | −0.183 | −1.954 | −1.088 | −0.788 | −1.420 | 825 | 11488931 |
| flurandrenolide | 20 | −0.657 | 0.660 | −1.532 | −0.468 | −0.175 | −0.391 | 0.147 | −0.012 | 0.506 | 826 | 11488792 |
| suxibuzone | 9.12 | 0.782 | 0.650 | −1.407 | −0.303 | 1.080 | 0.217 | 0.047 | 0.066 | 0.009 | 827 | 11467806 |
| suxibuzone | 20 | 0.036 | 0.114 | −1.112 | 0.149 | 0.446 | 0.786 | −0.032 | 0.342 | −0.704 | 827 | 11488782 |
| gossypol | 7.72 | −1.515 | 0.664 | −1.899 | −1.136 | 0.616 | 0.050 | 0.116 | 0.403 | −0.474 | 829 | 11467825 |
| gossypol-acetic acid complex | 20 | −1.769 | 0.687 | −0.514 | −0.562 | 0.036 | −0.783 | −0.993 | −0.832 | −1.124 | 829 | 11489288 |
| gossypol | 20 | −1.858 | −0.945 | −1.094 | −1.739 | −0.831 | −0.344 | 0.481 | 0.719 | −0.055 | 829 | 11489440 |
| pyrilamine | 14.02 | 0.003 | 0.003 | −0.365 | −0.643 | 0.610 | 0.189 | −0.112 | −0.200 | 0.097 | 830 | 11467437 |
| pyrilamine | 20 | −0.394 | −0.177 | −0.681 | −1.143 | 0.082 | −0.520 | −0.339 | −0.270 | −0.421 | 830 | 11489122 |
| aminothiazole | 20 | 0.434 | −0.453 | 0.282 | −0.203 | −0.192 | −0.795 | 0.910 | 0.981 | 0.567 | 831 | 11488695 |
| 1,3-dipropyl-8-cyclopentylxanthine | 20 | −0.311 | −0.355 | −1.261 | −0.485 | 0.379 | −0.922 | −1.351 | −1.137 | −1.470 | 832 | 11489624 |
| timolol | 20 | −1.197 | 0.603 | −1.677 | −0.712 | 0.291 | 0.183 | −0.502 | −0.341 | −0.725 | 833 | 11489150 |
| bethanechol | 24.82 | −0.527 | 0.785 | −0.769 | −0.302 | 0.403 | 0.427 | −0.146 | −0.255 | 0.087 | 834 | 11468221 |
| bethanechol | 20 | −0.241 | −0.837 | −0.432 | −0.617 | 0.600 | 0.461 | −0.002 | −0.234 | 0.421 | 834 | 11487948 |
| aceclidine | 20 | −0.803 | −0.537 | −1.930 | −0.779 | 0.704 | 0.088 | −1.118 | −1.208 | −0.638 | 835 | 11489051 |
| racephedrine | 20 | −0.482 | 0.052 | −0.260 | −1.006 | 0.267 | −0.266 | −0.174 | −0.169 | −0.151 | 836 | 11489125 |
| ethoxyquin | 18.4 | −0.683 | −0.391 | −1.947 | −0.140 | −1.587 | 0.143 | −0.796 | −0.799 | −0.640 | 837 | 11467913 |
| ethoxyquin | 20 | −0.755 | 0.497 | −1.530 | −0.482 | −0.693 | −0.284 | −0.318 | −0.536 | 0.190 | 837 | 11489200 |
| oxybenzone | 17.52 | −0.183 | 2.061 | 0.413 | 0.031 | −0.335 | −0.524 | −0.344 | −0.303 | −0.371 | 838 | 11468035 |
| oxybenzone | 20 | 0.101 | −0.222 | −1.049 | −1.001 | 0.569 | 0.440 | −0.803 | −0.675 | −0.836 | 838 | 11488824 |
| acyclovir | 17.76 | 0.037 | 0.945 | −0.982 | −0.750 | −0.531 | −0.016 | −0.140 | −0.071 | −0.299 | 839 | 11467234 |
| acyclovir | 20 | −1.461 | 0.290 | −1.615 | −0.907 | 0.478 | 1.195 | −0.497 | −0.363 | −0.667 | 839 | 11489379 |
| nafcillin | 20 | −0.907 | 0.089 | −2.111 | −0.682 | 0.769 | 0.010 | 0.655 | 0.580 | 0.727 | 840 | 11488253 |
| benfotiamine | 20 | 0.558 | 0.946 | −1.712 | −0.514 | −0.288 | −0.291 | −0.052 | 0.056 | −0.268 | 841 | 11489341 |
| methimazole | 20 | −0.250 | −0.309 | −0.088 | −0.276 | 0.484 | 1.745 | −0.212 | −0.013 | −0.500 | 842 | 11489089 |
| desipramine | 15.02 | 0.006 | −0.720 | −1.416 | −0.586 | 1.201 | −1.172 | −1.845 | −1.825 | −1.511 | 844 | 11467491 |
| desipramine | 20 | −0.166 | −1.007 | −1.581 | 0.147 | −0.521 | −0.230 | 0.029 | 0.087 | −0.154 | 844 | 11487907 |
| ritanserin | 20 | 0.584 | −1.308 | −0.230 | 1.734 | 0.481 | −0.198 | −0.452 | −0.391 | −0.478 | 846 | 11489376 |
| nerol | 20 | −1.107 | −0.034 | −0.445 | −0.485 | 0.605 | 0.242 | −0.601 | −0.604 | −0.484 | 847 | 11488600 |
| hydrocortisone acetate | 20 | −0.916 | 0.132 | −0.972 | 0.474 | 0.426 | −1.461 | 0.558 | 0.403 | 0.836 | 848 | 11488846 |
| trazodone | 10.76 | −0.035 | 0.143 | −1.152 | −0.093 | 0.052 | 0.028 | −0.711 | −0.542 | −0.913 | 850 | 11467440 |
| trazodone | 20 | −0.555 | −0.050 | −0.933 | −0.470 | 0.256 | 0.038 | −1.084 | −0.904 | −1.252 | 850 | 11488670 |
| ethaverine | 10.12 | 2.811 | −0.212 | −1.204 | −0.070 | −0.384 | −1.076 | −0.177 | −0.160 | −0.179 | 852 | 11467978 |
| ethaverine | 20 | 2.154 | −1.478 | −2.323 | 0.996 | −0.501 | −0.474 | −0.388 | −0.389 | −0.304 | 852 | 11489201 |
| aminophylline | 22.2 | −0.239 | 0.608 | −0.274 | 0.309 | 0.982 | 0.177 | −0.347 | −0.287 | −0.406 | 856 | 11467968 |
| theophylline | 22.2 | −1.365 | −0.560 | −1.045 | −0.775 | 0.571 | −0.061 | −0.063 | −0.107 | 0.042 | 856 | 11468021 |
| theophylline | 20 | 0.374 | −0.122 | −0.436 | 0.250 | 0.062 | 0.379 | 0.260 | 0.295 | 0.114 | 856 | 11488658 |
| benzyl benzoate | 20 | 0.328 | −0.081 | −0.729 | −0.596 | −0.086 | 0.394 | −0.734 | −0.765 | −0.489 | 857 | 11488348 |
| dropropizine | 16.92 | −0.965 | 1.439 | −0.480 | −0.580 | 0.111 | 0.335 | 0.283 | 0.417 | −0.044 | 858 | 11467393 |
| dropropizine | 20 | −1.041 | 0.541 | −1.015 | 0.418 | 0.422 | 1.315 | −0.985 | −0.814 | −1.068 | 858 | 11488781 |
| cyproterone acetate | 20 | 0.325 | 0.041 | 0.289 | −0.603 | 0.917 | −0.625 | 0.003 | −0.497 | 1.096 | 859 | 11489086 |
| pyridostigmine | 20 | −0.051 | 1.178 | −0.077 | −0.994 | −0.162 | 0.591 | −0.631 | −0.421 | −0.947 | 860 | 11488677 |
| captopril | 20 | 0.422 | −0.540 | −0.989 | −0.275 | 0.598 | 0.819 | −0.605 | −0.262 | −1.112 | 861 | 11489027 |
| cetrimonium | 20 | −4.937 | −8.079 | −5.775 | −3.527 | −2.664 | −4.872 | −1.380 | −2.697 | 1.657 | 862 | 11488246 |
| 1-[(4-chlorophenyl)phenyl-methyl]-4-methylpiperazine | 13.3 | −1.138 | −0.108 | −1.566 | −0.780 | 1.886 | 0.675 | −0.214 | −0.260 | −0.085 | 863 | 11467854 |
| THIP | 28.54 | 0.156 | 0.846 | −0.213 | 0.063 | 0.109 | −0.673 | −0.432 | −0.377 | −0.473 | 864 | 11468120 |
| gaboxadol | 20 | −0.610 | −0.252 | 0.416 | −0.077 | 0.376 | 0.752 | 0.326 | 0.228 | 0.472 | 864 | 11489406 |
| tolmetin | 15.54 | −1.012 | −0.410 | −1.908 | −0.662 | 0.814 | 0.088 | 0.367 | 0.391 | 0.234 | 865 | 11468004 |
| tolmetin | 20 | 0.563 | 0.424 | −1.321 | 0.135 | 0.316 | 0.182 | −0.091 | −0.029 | −0.133 | 865 | 11489021 |
| dinitolmide | 20 | −1.655 | 0.397 | −0.993 | −1.043 | 0.711 | 0.218 | 0.063 | 0.097 | −0.028 | 866 | 11488493 |
| sulfapyridine | 16.04 | −0.448 | −0.546 | −1.846 | −0.611 | 0.402 | 0.167 | −0.242 | −0.216 | −0.258 | 867 | 11467910 |
| sulfapyridine | 20 | −0.646 | −0.315 | −0.450 | −0.890 | −0.698 | −0.028 | −0.603 | −0.736 | −0.211 | 867 | 11489139 |
| ethosuximide | 28.34 | 0.606 | 1.098 | −0.713 | −0.159 | 0.812 | 0.241 | −0.154 | −0.111 | −0.257 | 869 | 11467313 |
| ethosuximide | 20 | −0.439 | 0.423 | −1.120 | −0.581 | −0.295 | 0.955 | −0.096 | −0.032 | −0.199 | 869 | 11489299 |
| alpha-cyano-4-hydroxycinnamic acid | 20 | −0.715 | 0.044 | −0.415 | −0.897 | 1.528 | 0.428 | 0.977 | 1.207 | 0.273 | 870 | 11489763 |
| sulconazole | 10.06 | 2.207 | 0.795 | −0.878 | 0.548 | −0.780 | −1.787 | 0.114 | 0.143 | 0.024 | 871 | 11467958 |
| sulconazole | 20 | −0.615 | −0.181 | −1.687 | 0.306 | 0.623 | 0.252 | 0.111 | −0.068 | 0.453 | 871 | 11489238 |
| adiphenine | 12.84 | −0.958 | 0.475 | −1.815 | −0.767 | 1.848 | 0.005 | −0.347 | −0.337 | −0.337 | 872 | 11467223 |
| drofenine | 12.6 | −0.398 | −0.672 | −0.071 | −0.725 | 1.212 | −0.555 | −0.686 | −0.839 | −0.237 | 872 | 11467937 |
| drofenine | 20 | −0.776 | 1.146 | −1.273 | −0.478 | 0.766 | −0.459 | −0.215 | −0.080 | −0.378 | 872 | 11488791 |
| adiphenine | 20 | 0.748 | 0.187 | −0.046 | −0.414 | 0.462 | −0.052 | 0.746 | 0.994 | 0.100 | 872 | 11489333 |
| folinic acid | 8.44 | 0.225 | 0.385 | −1.631 | −0.787 | 0.626 | 0.217 | −0.201 | 0.073 | −0.715 | 873 | 11467886 |
| leucovorin | 20 | −0.829 | −0.537 | −1.686 | −0.840 | 1.147 | 0.710 | 0.546 | 0.533 | 0.404 | 873 | 11487932 |
| alanyl-DL-leucine | 20 | −0.659 | 1.172 | −0.857 | −0.474 | 0.234 | 0.062 | −0.233 | −0.145 | −0.365 | 874 | 11489170 |
| oxytetracycline | 8.68 | 0.029 | −0.781 | −1.129 | −1.245 | 0.484 | −1.500 | 0.442 | 0.110 | 1.021 | 876 | 11467455 |
| oxytetracycline | 20 | 0.398 | 1.384 | −1.134 | 0.039 | 0.702 | −0.052 | −0.090 | −0.272 | 0.372 | 876 | 11488804 |
| clofibric acid | 18.64 | 0.273 | −0.471 | −0.958 | −0.372 | −0.088 | 0.840 | −0.231 | 0.097 | −0.837 | 877 | 11467931 |
| clofibric acid | 20 | −0.594 | 0.332 | −0.645 | −0.446 | 1.577 | 0.364 | 0.503 | 0.640 | 0.066 | 877 | 11487973 |
| sulfacetamide | 18.68 | −0.387 | 0.792 | −0.835 | −0.530 | 0.957 | −0.793 | −1.595 | −1.634 | −1.255 | 878 | 11467162 |
| sulfacetamide | 20 | −0.436 | 0.539 | −1.601 | −0.433 | 0.916 | 0.168 | −0.496 | −0.528 | −0.331 | 878 | 11489134 |
| norepinephrine | 20 | −0.497 | 0.896 | −1.680 | −1.218 | 0.595 | −0.296 | 0.129 | 0.343 | −0.251 | 879 | 11488880 |
| hydrocortisone sodium phosphate | 20 | −0.433 | 0.163 | −0.338 | 0.227 | 1.136 | −0.963 | −1.063 | −1.143 | −0.605 | 881 | 11488836 |
| azithromycin | 20 | 0.362 | −0.058 | −1.400 | 0.138 | 0.213 | −0.272 | 0.145 | 0.281 | −0.150 | 882 | 11489398 |
| phenethicillin | 10.98 | 0.583 | 1.325 | −0.792 | −0.639 | −0.167 | −0.388 | −0.070 | −0.044 | −0.116 | 883 | 11467871 |
| phenethicillin | 20 | −1.294 | −0.099 | −0.749 | −0.681 | −0.056 | 1.599 | 0.385 | 0.572 | −0.074 | 883 | 11489153 |
| pheniramine | 16.64 | −1.261 | 0.159 | −1.080 | −0.624 | 0.807 | 0.142 | −0.600 | −0.558 | −0.606 | 884 | 11467207 |
| pheniramine | 20 | −0.297 | −0.332 | −0.677 | −1.027 | 1.461 | 0.030 | 0.015 | 0.052 | 0.005 | 884 | 11489093 |
| amoxepine | 12.74 | −0.392 | 0.116 | −1.674 | 0.000 | 0.687 | −3.399 | −0.163 | −0.119 | −0.270 | 885 | 11467250 |
| amoxepine | 20 | −1.525 | −3.135 | −4.378 | 0.225 | −1.877 | −0.073 | −0.415 | −0.391 | −0.305 | 885 | 11489061 |
| cinchonine | 20 | −1.015 | 0.396 | −2.294 | −0.760 | 0.846 | −0.198 | −0.505 | −0.508 | −0.354 | 886 | 11488410 |
| sulfamethoxypyridazine | 14.28 | −0.260 | 2.118 | −1.443 | −0.906 | −0.551 | 0.465 | −0.244 | −0.137 | −0.423 | 887 | 11467872 |
| sulfamethoxypyridazine | 20 | −1.373 | −0.276 | −1.140 | −0.525 | 1.086 | 1.355 | 0.031 | −0.178 | 0.457 | 887 | 11489245 |
| isopropamide | 11.32 | −0.781 | −0.113 | −1.046 | 0.074 | 0.128 | 0.760 | −0.445 | −0.399 | −0.453 | 888 | 11467918 |
| isopropamide | 20 | 0.500 | 0.121 | −0.876 | −0.072 | 1.374 | −0.890 | 0.557 | 0.643 | 0.333 | 888 | 11488867 |
| pyrazinamide | 32.5 | −1.021 | 0.394 | −1.589 | −0.899 | 1.562 | 0.315 | −0.920 | −0.707 | −1.178 | 889 | 11467662 |
| pyrazinamide | 20 | −1.060 | −0.059 | −0.218 | −0.672 | −0.041 | −0.043 | −0.560 | −0.564 | −0.444 | 889 | 11489121 |
| (R)-naproxen sodium salt | 17.38 | 0.632 | 0.405 | 0.325 | −0.253 | −0.736 | 0.444 | −0.254 | 0.135 | −0.984 | 890 | 11467939 |
| naproxen | 20 | 0.029 | 1.749 | 0.134 | 0.903 | 0.583 | 0.709 | −0.468 | −0.192 | −0.871 | 890 | 11488859 |
| desoxycorticosterone acetate | 20 | 0.272 | 0.757 | −0.903 | −0.625 | 1.339 | 0.070 | −0.054 | −0.073 | 0.039 | 891 | 11488232 |
| acriflavinium hydrochloride | 20 | −0.949 | −4.061 | −4.725 | −4.924 | 5.023 | 6.421 | −0.846 | 0.240 | −2.906 | 892 | 11487882 |
| octopamine | 26.12 | −1.084 | −0.385 | −0.201 | 0.058 | 0.440 | 0.632 | 0.462 | 0.113 | 1.082 | 893 | 11468097 |
| octopamine | 20 | 0.482 | 0.006 | −0.664 | −0.763 | −0.323 | −0.649 | −0.237 | −0.111 | −0.504 | 893 | 11488038 |
| cyclophosphamide | 20 | 0.038 | 1.271 | −0.818 | −0.026 | 1.115 | −0.088 | −0.794 | −0.800 | −0.548 | 894 | 11488962 |
| naringin | 6.9 | −0.074 | −0.461 | −1.163 | −0.555 | 0.688 | −0.005 | −0.196 | −0.153 | −0.241 | 895 | 11467615 |
| guaifenesin | 20.18 | −0.012 | −0.442 | −1.923 | −0.888 | −0.163 | 0.480 | −0.687 | −0.558 | −0.811 | 896 | 11467924 |
| guaifenesin | 20 | −1.514 | 0.515 | −1.306 | −1.012 | 0.746 | −0.165 | 0.089 | 0.196 | −0.069 | 896 | 11488920 |
| retinyl palmitate | 20 | −0.943 | 0.219 | −1.690 | −0.892 | 1.074 | −0.170 | −0.601 | −0.509 | −0.672 | 897 | 11489380 |
| acetyl tyrosine ethyl ester | 20 | −0.951 | −0.040 | −1.391 | −0.587 | 0.279 | 0.144 | −0.191 | −0.294 | 0.068 | 898 | 11489161 |
| apomorphine | 14.96 | 0.289 | −0.520 | −0.641 | −1.080 | −0.448 | 0.262 | −0.467 | −0.342 | −0.685 | 899 | 11467249 |
| tenoxicam | 11.86 | −0.940 | −0.251 | −1.037 | −0.912 | 1.391 | 0.017 | −0.797 | −0.761 | −0.724 | 900 | 11467675 |
| tenoxicam | 20 | −0.463 | −0.188 | −0.698 | −0.118 | 1.260 | −0.263 | −0.518 | −0.572 | −0.236 | 900 | 11488896 |
| chlortetracycline | 8.36 | −0.009 | −0.150 | −0.740 | −0.228 | 0.234 | 1.470 | 0.221 | 0.150 | 0.275 | 901 | 11467293 |
| chlortetracycline | 20 | 0.391 | 0.875 | 0.026 | 0.339 | −0.390 | 0.005 | −0.076 | 0.198 | −0.636 | 901 | 11488618 |
| furegrelate | 20 | −0.903 | 1.494 | −1.018 | −0.500 | −0.898 | 0.520 | 0.166 | 0.267 | −0.085 | 902 | 11489260 |
| fenbendazole | 20 | −0.398 | −1.895 | −3.769 | −0.360 | −0.797 | −1.535 | 0.339 | 0.350 | 0.186 | 903 | 11487856 |
| piracetam | 28.14 | −0.740 | 0.674 | −1.080 | −0.509 | 0.945 | 0.118 | −0.513 | −0.413 | −0.612 | 904 | 11467685 |
| piracetam | 20 | −1.349 | −0.321 | −1.691 | −0.835 | 0.359 | −0.008 | −0.486 | −0.162 | −0.974 | 904 | 11488890 |
| novobiocin | 20 | −0.074 | 1.360 | −1.637 | −0.409 | 0.912 | 0.178 | −0.266 | −0.148 | −0.371 | 905 | 11488793 |
| glucosamine | 20 | −0.796 | −0.122 | −0.765 | 0.282 | 0.670 | −0.446 | −0.597 | −0.792 | −0.019 | 906 | 11488335 |
| xanthurenic acid | 20 | 0.001 | 0.312 | −0.448 | −0.328 | 2.263 | 0.322 | 1.482 | 0.929 | 2.255 | 907 | 11487974 |
| berberine | 11.9 | −1.320 | −3.912 | −3.349 | −1.161 | −1.789 | −1.873 | −0.836 | 0.473 | −3.343 | 909 | 11467734 |
| berberine | 20 | −1.268 | −4.055 | −2.731 | −0.022 | −2.291 | −1.103 | −2.301 | −0.630 | −5.281 | 909 | 11488710 |
| metergoline | 20 | 2.185 | 1.154 | −0.394 | 0.542 | −0.322 | 0.464 | −0.831 | −0.817 | −0.720 | 910 | 11488698 |
| tuaminoheptane | 20 | −0.503 | 0.784 | −0.807 | −0.599 | 0.062 | 0.317 | −0.440 | −0.446 | −0.277 | 911 | 11488363 |
| propylthiouracil | 23.5 | 0.265 | 0.419 | −1.364 | −0.885 | 0.550 | 0.762 | −0.336 | −0.037 | −0.879 | 912 | 11467642 |
| propylthiouracil | 20 | −1.184 | 0.081 | −0.209 | −0.455 | 0.730 | 0.717 | −0.187 | −0.166 | −0.205 | 912 | 11489118 |
| uridine triphosphate | 20 | −1.493 | 0.343 | −1.167 | −0.931 | 0.710 | 0.008 | −0.698 | −0.758 | −0.392 | 913 | 11488341 |
| aloin | 20 | −0.202 | 0.866 | −0.001 | −1.874 | 1.317 | 0.723 | 0.325 | 0.339 | 0.195 | 914 | 11489753 |
| diclofenac | 13.5 | −0.577 | −0.773 | −1.841 | −0.575 | 0.550 | 0.770 | −0.040 | −0.136 | 0.163 | 915 | 11467742 |
| diclofenac | 20 | −0.153 | −0.098 | −1.012 | −0.809 | −0.726 | 0.353 | 0.156 | 0.356 | −0.208 | 915 | 11488807 |
| bendroflumethiazide | 9.5 | −0.260 | −0.339 | −0.886 | −0.479 | 0.375 | 0.532 | −0.350 | −0.149 | −0.693 | 917 | 11467932 |
| bendrofumethiazide | 20 | 0.545 | 0.789 | −0.808 | −0.223 | 0.027 | 0.025 | −0.175 | −0.224 | −0.052 | 917 | 11489340 |
| metolazone | 10.94 | −1.024 | 0.295 | −1.290 | −0.645 | −0.232 | 0.416 | −0.834 | −0.884 | −0.613 | 918 | 11467260 |
| metolazone | 20 | −0.785 | 0.519 | −0.787 | −0.295 | 1.525 | 0.250 | −1.310 | −1.006 | −1.631 | 918 | 11489557 |
| sulpiride | 11.72 | −1.374 | 0.210 | −1.069 | −1.106 | 1.072 | −0.307 | −0.479 | −0.274 | −0.848 | 920 | 11467204 |
| hexetidine | 11.78 | 0.466 | 0.041 | −0.445 | −0.819 | 0.506 | 1.554 | −0.199 | −0.254 | −0.053 | 922 | 11467699 |
| hexetidine | 20 | −0.103 | 0.307 | −0.144 | −0.178 | 0.543 | 0.428 | −0.359 | −0.132 | −0.761 | 922 | 11488769 |
| allantoin | 25.3 | 0.126 | 1.392 | −0.125 | 0.291 | 0.670 | −0.389 | −1.230 | −1.136 | −1.233 | 923 | 11467150 |
| allantoin | 20 | 0.212 | 0.038 | −0.707 | −0.178 | 0.360 | 0.829 | 0.901 | 0.865 | 0.732 | 923 | 11488035 |
| 1-phenylbiguanide | 20 | −0.283 | 0.801 | −0.680 | 0.254 | 0.605 | −1.113 | 0.136 | 0.032 | 0.397 | 924 | 11489006 |
| N-methyl (−)ephedrine | 20 | 0.576 | 0.721 | 0.526 | −0.068 | 1.190 | 0.065 | 0.292 | 0.437 | 0.018 | 925 | 11489012 |
| dantron | 20 | −1.019 | 0.712 | −1.565 | −1.152 | 1.067 | 0.404 | −0.515 | −0.457 | −0.492 | 926 | 11488419 |
| clemastine | 11.64 | −0.611 | −0.127 | −0.699 | 0.029 | 0.597 | −0.693 | 0.134 | 0.024 | 0.344 | 927 | 11467454 |
| clemastine | 20 | −1.946 | −1.286 | −0.522 | −0.630 | −0.038 | −0.437 | −0.961 | −0.954 | −0.813 | 927 | 11488505 |
| phenylmercuric acetate | 20 | −5.759 | −7.229 | −6.035 | −3.739 | −3.333 | −3.937 | ND | ND | ND | 928 | 11488759 |
| naloxone | 12.22 | −0.717 | 0.222 | −0.356 | −0.181 | 0.124 | 0.610 | −0.445 | −0.271 | −0.753 | 929 | 11467259 |
| tolperisone | 20 | 0.208 | 0.166 | 0.121 | −0.742 | 0.169 | 0.231 | 0.266 | 0.282 | 0.170 | 930 | 11488667 |
| hydrochlorothiazide | 13.44 | −0.128 | 2.087 | 0.208 | 0.055 | 0.150 | −0.784 | 0.006 | 0.079 | −0.180 | 931 | 11467157 |
| hydrochlorothiazide | 20 | −0.515 | 1.005 | 0.142 | 0.081 | 1.061 | 0.678 | 0.121 | −0.077 | 0.566 | 931 | 11488856 |
| lysyl-tyrosyl-lysine acetate | 20 | 0.623 | 1.667 | −0.071 | 0.025 | −0.231 | 0.305 | 0.044 | 0.265 | −0.362 | 932 | 11488370 |
| scopolamine | 20 | −0.457 | 0.222 | −1.061 | −0.720 | −0.305 | 0.547 | −0.130 | −0.060 | −0.236 | 933 | 11489129 |
| sulfamethazine | 14.38 | −0.189 | 0.662 | −2.022 | −0.527 | 0.284 | 0.504 | −0.263 | −0.192 | −0.359 | 934 | 11467923 |
| sulfamethazine | 20 | 0.400 | 0.399 | 0.144 | −0.945 | 0.049 | 0.045 | −0.040 | −0.110 | 0.105 | 934 | 11489137 |
| erythromycin | 20 | −1.364 | 0.625 | −0.660 | −0.336 | 1.091 | 0.042 | −0.155 | −0.183 | −0.086 | 935 | 11488575 |
| erythromycin stearate | 20 | −0.693 | 1.313 | −0.735 | −0.415 | 0.248 | 0.377 | −0.765 | −0.506 | −1.068 | 935 | 11489079 |
| glafenine | 10.72 | 0.145 | 0.341 | −1.287 | −0.299 | 0.895 | 0.046 | −0.938 | −0.772 | −1.096 | 936 | 11467441 |
| glafenine | 20 | 0.436 | 0.057 | −1.536 | −0.282 | 0.248 | 0.289 | 0.070 | 0.100 | 0.000 | 936 | 11489199 |
| propiomazine | 20 | −0.631 | −0.190 | −0.200 | −0.457 | 1.024 | −0.368 | −0.901 | −0.922 | −0.695 | 937 | 11488746 |
| triprolidine | 14.36 | −0.362 | −0.343 | −1.007 | −0.687 | 0.468 | −0.487 | 0.398 | 0.457 | 0.219 | 938 | 11467410 |
| triprolidine | 20 | −1.177 | −0.079 | −0.841 | −0.733 | 0.106 | 0.464 | −1.449 | −1.407 | −1.272 | 938 | 11488661 |
| mefenamic acid | 16.58 | −0.200 | −0.654 | −1.115 | −0.874 | 1.232 | 0.645 | −0.921 | −0.866 | −0.885 | 939 | 11467202 |
| mefenamic acid | 20 | 0.265 | 0.808 | −1.055 | 0.343 | 0.812 | −0.377 | −0.010 | 0.038 | −0.148 | 939 | 11489757 |
| oxyphenbutazone | 12.34 | 1.015 | 1.839 | 1.101 | −0.345 | −1.738 | 0.009 | −1.275 | −1.009 | −1.587 | 943 | 11468197 |
| oxyphenbutazone | 20 | −0.624 | 0.112 | −0.740 | −0.455 | −1.341 | −0.261 | 0.468 | 0.632 | −0.022 | 943 | 11487969 |
| sulfaphenazole | 12.72 | 0.008 | 0.615 | −1.364 | −0.150 | 0.517 | 0.259 | −0.733 | −0.478 | −1.156 | 944 | 11467169 |
| sulfaphenazole | 20 | −0.557 | −0.222 | −1.608 | −0.517 | 0.457 | 0.385 | −0.702 | −0.808 | −0.398 | 944 | 11489759 |
| flumethasone | 20 | −1.119 | 0.128 | −1.641 | −0.276 | 0.552 | 0.021 | −0.255 | −0.332 | 0.042 | 945 | 11489081 |
| etanidazole | 18.68 | 0.304 | 1.433 | −0.755 | −0.106 | 0.456 | −0.352 | −0.518 | −0.176 | −1.102 | 946 | 11467797 |
| etanidazole | 20 | 0.378 | 0.399 | −0.963 | 0.547 | 0.710 | 0.585 | 0.484 | 0.559 | 0.207 | 946 | 11488726 |
| phenindione | 18 | −0.467 | −0.314 | −1.060 | −0.884 | 0.415 | −0.504 | 0.268 | 0.384 | −0.018 | 948 | 11467686 |
| phenindione | 20 | −0.732 | 0.166 | −0.880 | −0.494 | 0.375 | −0.091 | 0.457 | 0.146 | 1.082 | 948 | 11488815 |
| kynurenic acid | 20 | −0.228 | 0.451 | −0.914 | −0.362 | −0.143 | 0.757 | −0.292 | −0.194 | −0.436 | 949 | 11489158 |
| parachlorophenol | 20 | 0.247 | 2.451 | −0.668 | 0.782 | 1.436 | 0.036 | −0.158 | −0.090 | −0.190 | 950 | 11488784 |
| biotin | 16.38 | −0.264 | 1.174 | −1.048 | −0.435 | 0.639 | 0.776 | 0.069 | 0.422 | −0.659 | 951 | 11467566 |
| penicillamine | 20 | −0.469 | −0.743 | −0.735 | −0.399 | 0.702 | −0.400 | −0.544 | −0.497 | −0.454 | 952 | 11488845 |
| levonordefrin | 20 | −0.402 | 0.477 | −1.614 | −0.842 | 1.422 | −0.288 | 0.626 | 0.780 | 0.260 | 953 | 11488871 |
| benzylpenicillin | 11.96 | −1.082 | −0.475 | −0.542 | 0.043 | −0.022 | −0.688 | −0.223 | −0.276 | −0.080 | 954 | 11468226 |
| benzyl penicillin | 20 | −1.323 | −0.169 | −1.364 | −0.496 | 0.551 | −0.771 | 0.290 | 0.219 | 0.420 | 954 | 11488334 |
| bromopride | 11.62 | −0.964 | −0.735 | −1.318 | −0.682 | 0.648 | 0.556 | 0.137 | 0.141 | 0.113 | 955 | 11467852 |
| bromopride | 20 | 1.305 | 1.442 | −0.793 | −0.703 | 0.412 | 0.066 | 0.183 | 0.272 | −0.040 | 955 | 11489343 |
| cinoxacin | 15.26 | 0.349 | 0.158 | −0.562 | −0.752 | 0.598 | 0.136 | −0.196 | −0.293 | 0.036 | 956 | 11467928 |
| cinoxacin | 20 | 0.595 | −0.056 | −0.785 | 1.067 | 0.254 | −0.430 | 0.536 | 0.498 | 0.544 | 956 | 11488386 |
| azaserine | 20 | 0.962 | 0.847 | −0.958 | −0.570 | 0.951 | 1.220 | 0.010 | 0.142 | −0.205 | 957 | 11489037 |
| phenacemide | 20 | −0.803 | −0.405 | −0.661 | −0.270 | 0.506 | −0.638 | −0.628 | −0.528 | −0.628 | 958 | 11488835 |
| papaverine | 11.78 | −0.036 | −0.710 | −1.936 | −1.024 | −0.653 | 0.213 | 0.042 | 0.201 | −0.293 | 959 | 11467731 |
| papaverine | 20 | 0.805 | 0.164 | −1.548 | −0.508 | 0.251 | 0.498 | 0.164 | 0.431 | −0.332 | 959 | 11488794 |
| methenamine | 20 | −0.695 | 0.559 | −1.407 | −0.763 | 0.752 | −0.288 | −0.224 | −0.092 | −0.465 | 960 | 11488643 |
| noscapine | 9.68 | −0.264 | 1.733 | −0.527 | −0.196 | 0.408 | 1.142 | 0.193 | 0.305 | −0.082 | 961 | 11467711 |
| primidone | 18.32 | 0.205 | 0.167 | −0.306 | −0.234 | −0.056 | 0.296 | −0.503 | −0.305 | −0.815 | 962 | 11468081 |
| primidone | 20 | −1.078 | 1.784 | −1.188 | −0.357 | −0.132 | 0.193 | −0.784 | −0.715 | −0.768 | 962 | 11489109 |
| piperacillin | 7.72 | −1.163 | −0.185 | −1.733 | −0.785 | 0.366 | 0.392 | −0.575 | −0.714 | −0.189 | 963 | 11467903 |
| dacarbazine | 21.96 | −1.008 | 0.529 | −1.457 | −0.988 | 0.573 | −1.444 | −0.588 | −0.551 | −0.559 | 964 | 11467722 |
| dacarbazine | 20 | −0.158 | 2.171 | −1.023 | 0.461 | 0.614 | 1.072 | −0.186 | −0.180 | −0.084 | 964 | 11488964 |
| tolazoline | 24.96 | −1.209 | −0.447 | −1.058 | −0.335 | 1.649 | 0.518 | 0.179 | −0.117 | 0.702 | 965 | 11467208 |
| tolazoline | 20 | 1.163 | 0.855 | −1.583 | −0.106 | −0.280 | 0.507 | −0.540 | −0.543 | −0.357 | 965 | 11489020 |
| gluconolactone | 20 | −0.071 | 2.069 | −0.946 | −0.537 | −0.031 | 0.618 | −0.720 | −0.830 | −0.390 | 966 | 11489749 |
| beta-carotene | 20 | −1.148 | 1.200 | −1.352 | −0.423 | 1.388 | 0.027 | −0.186 | −0.130 | −0.192 | 967 | 11489072 |
| phenylbutazone | 20 | −0.411 | 1.825 | −0.468 | 0.867 | 0.633 | 0.755 | −0.952 | −0.826 | −1.026 | 968 | 11489098 |
| dibucaine | 11.64 | −0.456 | 0.524 | −0.789 | −0.398 | 0.810 | 0.922 | −0.769 | −0.761 | −0.681 | 969 | 11467224 |
| dibucaine | 20 | 0.902 | 0.183 | −0.032 | −0.701 | 0.770 | 0.026 | −0.230 | 0.091 | −0.771 | 969 | 11488817 |
| cineole | 20 | 0.454 | 0.218 | 0.033 | −0.396 | −0.452 | −0.082 | 0.552 | 1.081 | −0.689 | 970 | 11488037 |
| tolnaftate | 13.02 | −0.302 | −0.585 | −0.402 | 0.900 | 1.104 | −0.676 | −1.563 | −1.816 | −0.782 | 971 | 11467218 |
| tolnaftate | 20 | −0.237 | −0.027 | 0.442 | 1.065 | 0.949 | 0.429 | −0.311 | −0.306 | −0.284 | 971 | 11488766 |
| thiothixene | 20 | 0.036 | 0.795 | −1.708 | −0.227 | −0.324 | 0.307 | −0.215 | 0.004 | −0.624 | 972 | 11489149 |
| anisindione | 20 | −0.786 | −0.936 | −1.956 | −0.059 | −0.202 | 0.552 | −0.453 | −0.598 | −0.018 | 973 | 11488243 |
| nafronyl | 10.42 | −0.813 | −0.179 | −0.493 | −0.845 | 1.725 | −0.907 | 0.515 | 0.429 | 0.595 | 975 | 11467525 |
| nafronyl | 20 | −0.519 | 1.103 | −0.544 | −0.316 | 0.888 | −0.255 | −0.090 | −0.083 | −0.117 | 975 | 11488684 |
| eserine | 14.52 | −0.546 | 0.823 | −1.277 | −0.295 | 0.483 | −0.432 | −0.100 | 0.136 | −0.569 | 976 | 11467714 |
| eserine | 20 | 0.536 | 1.565 | 0.550 | 1.371 | 0.454 | 0.260 | 0.275 | 0.092 | 0.630 | 976 | 11488146 |
| physostigmine | 20 | −1.141 | 0.478 | −1.244 | −0.544 | 1.002 | 0.767 | 0.331 | 0.298 | 0.310 | 976 | 11488573 |
| physostigmine | 20 | −0.530 | 1.275 | −0.353 | 0.254 | 0.383 | 1.523 | −0.337 | −0.286 | −0.381 | 976 | 11489099 |
| triamcinolone | 20 | −1.408 | −0.080 | −1.074 | −0.458 | 0.484 | 0.087 | 0.217 | 0.162 | 0.261 | 977 | 11488765 |
| methacholine | 20 | −0.187 | 0.243 | −1.628 | −0.396 | 0.495 | 0.373 | −0.001 | −0.093 | 0.140 | 978 | 11487831 |
| pyrithione zinc | 20 | −5.339 | −8.322 | −6.284 | −3.529 | −2.405 | −4.773 | −3.250 | −4.140 | −0.770 | 979 | 11488778 |
| doxycycline | 20 | −0.322 | −0.568 | −1.407 | −1.355 | 0.943 | 0.435 | 0.215 | −0.344 | 1.238 | 980 | 11487959 |
| cetylpyridinium | 20 | −4.516 | −6.696 | −4.298 | −2.898 | −2.465 | −3.857 | −1.684 | −2.717 | 0.819 | 981 | 11488276 |
| bisacodyl | 11.06 | 0.534 | 0.591 | −0.647 | −0.429 | 0.834 | 0.628 | −0.168 | 0.044 | −0.566 | 982 | 11467567 |
| bisacodyl | 20 | −0.420 | −0.678 | −1.312 | −0.091 | 1.930 | −0.324 | 0.567 | 0.230 | 1.072 | 982 | 11487965 |
| 3-aminopropanesulphonic acid | 20 | −0.590 | 0.270 | −1.038 | −0.639 | 0.548 | 0.103 | −0.431 | −0.579 | −0.047 | 983 | 11489225 |
| medrysone | 20 | −0.119 | −0.757 | −0.168 | −0.229 | 0.617 | −0.214 | −0.345 | −0.260 | −0.501 | 984 | 11487957 |
| sodium p-aminosalicylate | 20 | −0.471 | 0.038 | 0.357 | 1.365 | −0.427 | −0.286 | −0.174 | −0.196 | −0.153 | 985 | 11487817 |
| creatinine | 20 | −0.905 | 0.482 | −1.890 | −0.752 | 0.502 | 0.775 | −0.847 | −0.777 | −0.833 | 986 | 11488580 |
| acetylglucosamine | 20 | 0.500 | 1.126 | −0.603 | −0.599 | −0.424 | 0.235 | −0.386 | −0.268 | −0.548 | 987 | 11489169 |
| melatonin | 17.22 | 0.105 | −0.726 | −1.330 | −0.631 | 0.057 | 0.187 | 0.017 | 0.046 | −0.047 | 988 | 11467606 |
| melatonin | 20 | −1.302 | 0.362 | −0.916 | −0.176 | 0.689 | −0.540 | −0.344 | −0.445 | −0.066 | 988 | 11489160 |
| arcaine | 23.22 | −0.483 | −1.017 | −0.864 | −0.118 | 0.458 | 0.289 | 0.548 | 0.657 | 0.223 | 989 | 11468024 |
| arcaine | 20 | −0.781 | 0.634 | −0.583 | −0.696 | 0.485 | 0.438 | −0.874 | −0.949 | −0.517 | 989 | 11488417 |
| carbetapentane | 12 | −1.812 | −0.069 | −0.968 | −1.071 | 1.499 | 0.181 | −0.201 | −0.168 | −0.223 | 990 | 11467535 |
| carbetapentane | 20 | −0.529 | 0.411 | −0.699 | −0.737 | 0.751 | 0.439 | −0.661 | −0.663 | −0.535 | 990 | 11489228 |
| methylergonovine | 20 | −0.522 | 0.484 | −1.322 | −1.113 | 0.608 | 0.135 | 0.134 | 0.157 | 0.107 | 991 | 11488429 |
| pilocarpine | 19.2 | −1.894 | −0.189 | −0.231 | −0.246 | 0.442 | 0.267 | −0.704 | −0.683 | −0.612 | 992 | 11467597 |
| pilocarpine | 20 | −0.982 | 0.951 | −0.296 | −0.022 | 0.553 | −0.617 | −1.869 | −1.707 | −1.840 | 992 | 11489100 |
| acetyltryptophanamide | 20 | −1.131 | 0.508 | −0.574 | −0.421 | 0.686 | −0.369 | 0.556 | 0.622 | 0.314 | 993 | 11489162 |
| canavanine | 20 | −0.464 | 0.587 | 0.856 | −0.257 | 1.121 | 0.405 | −0.073 | −0.267 | 0.320 | 994 | 11488616 |
| lincomycin | 20 | −0.335 | −0.348 | −1.812 | −0.433 | 0.555 | 0.226 | −0.263 | −0.365 | −0.048 | 995 | 11487921 |
| oxidopamine | 20 | −0.607 | 0.079 | −0.911 | −0.781 | 0.702 | −0.462 | −0.311 | −0.081 | −0.635 | 996 | 11488834 |
| mafenide | 21.48 | 0.579 | 0.875 | −1.132 | −0.541 | 0.901 | 0.193 | 0.457 | 0.570 | 0.096 | 997 | 11467314 |
| mafenide | 20 | 0.172 | −0.765 | −1.529 | −1.186 | 1.778 | 0.225 | 0.537 | 0.517 | 0.423 | 997 | 11487911 |
| suloctidil | 11.84 | 1.234 | −3.733 | −1.031 | −0.073 | −1.363 | −4.302 | −0.794 | −0.535 | −1.169 | 998 | 11467569 |
| suloctidil | 20 | −5.538 | −6.841 | −3.516 | 0.897 | −2.580 | −0.200 | −0.351 | −0.082 | −0.819 | 998 | 11489243 |
| lomefloxacin | 11.38 | −0.420 | −0.718 | −0.692 | −0.896 | 0.618 | −0.062 | −0.538 | −0.642 | −0.262 | 999 | 11467386 |
| lomefloxacin | 20 | −0.549 | 0.815 | −0.966 | −0.974 | 0.732 | −0.201 | −0.884 | −0.989 | −0.502 | 999 | 11488512 |
| trichlormethiazide | 10.5 | 0.337 | 0.313 | −1.914 | −0.867 | 1.134 | 0.012 | −0.132 | −0.198 | 0.022 | 1000 | 11467973 |
| trichlormethiazide | 20 | −0.452 | −0.172 | −0.230 | −0.474 | 1.427 | 0.482 | −0.656 | −0.725 | −0.393 | 1000 | 11488764 |
| meclofenoxate | 15.52 | −0.665 | 0.254 | −2.937 | −0.681 | 0.244 | 0.233 | −0.284 | −0.265 | −0.279 | 1001 | 11467911 |
| meclofenoxate | 20 | −1.088 | −0.199 | −2.037 | −0.865 | 1.685 | 0.206 | 0.669 | 0.577 | 0.777 | 1001 | 11488274 |
| diphenhydramine | 15.66 | −1.330 | −0.459 | −1.616 | −1.065 | 1.420 | 0.902 | −0.485 | −0.763 | 0.139 | 1002 | 11467213 |
| diphenhydramine | 20 | −0.734 | 0.678 | −0.657 | 0.951 | 0.696 | 0.018 | −0.620 | −0.450 | −0.860 | 1002 | 11488777 |
| 7,8-dihydroxyflavone | 20 | −0.560 | 0.069 | −0.633 | −0.861 | −0.473 | 0.609 | 1.304 | 1.580 | 0.448 | 1004 | 11488768 |
| trihexyphenidyl | 13.26 | −0.848 | 0.293 | −0.699 | −0.642 | 1.169 | −0.131 | −0.397 | −0.346 | −0.423 | 1005 | 11467849 |
| pridinol | 13.54 | 0.737 | 0.046 | 0.600 | −0.254 | 0.436 | 0.272 | 0.180 | 0.073 | 0.369 | 1005 | 11467947 |
| trihexyphenidyl | 20 | −0.546 | −0.482 | −0.323 | −0.583 | 0.673 | 0.359 | −0.224 | −0.192 | −0.269 | 1005 | 11488645 |
| pridinol | 20 | −1.349 | −0.694 | −1.080 | −1.043 | 0.304 | −0.767 | 0.892 | 1.147 | 0.188 | 1005 | 11489801 |
| cytarabine | 20 | 0.335 | 0.225 | −1.460 | −0.640 | 2.243 | 0.321 | 1.548 | 1.214 | 1.864 | 1006 | 11487975 |
| L(−)-vesamicol | 15.42 | −1.137 | 0.104 | 0.241 | 0.219 | −0.265 | −0.597 | −0.847 | −0.941 | −0.491 | 1008 | 11468068 |
| methscopolamine | 20 | −0.033 | 0.066 | −1.244 | −0.165 | 0.226 | −0.203 | 0.220 | 0.140 | 0.430 | 1009 | 11488878 |
| trioxsalen | 17.52 | −0.830 | −0.717 | −1.142 | 0.059 | 0.920 | 0.088 | 0.087 | 0.279 | −0.302 | 1012 | 11467857 |
| trioxsalen | 20 | 0.083 | 0.598 | −1.440 | −0.460 | 0.909 | 0.322 | −0.828 | −0.854 | −0.541 | 1012 | 11488899 |
| cresol | 20 | −0.895 | 0.322 | −1.469 | −1.127 | 0.649 | 0.808 | −0.382 | −0.326 | −0.433 | 1013 | 11488581 |
| nefopam | 15.78 | −0.091 | −0.874 | −1.042 | −0.688 | 0.686 | 0.225 | 0.106 | −0.140 | 0.543 | 1016 | 11467377 |
| nefopam | 20 | −0.789 | 0.061 | −1.349 | −1.142 | 1.277 | 0.349 | −0.857 | −0.672 | −1.059 | 1016 | 11489232 |
| acetyltryptophan | 20 | −0.798 | −0.284 | −0.813 | −0.592 | 0.503 | −0.382 | −0.550 | −0.550 | −0.440 | 1017 | 11489164 |
| dextromethorphan | 20 | 0.075 | −0.432 | −0.746 | −0.515 | 0.358 | 0.395 | 0.241 | 0.373 | −0.003 | 1018 | 11488837 |
| carbamazepine | 16.92 | 1.020 | 0.385 | −1.902 | −0.344 | 0.805 | 0.360 | −1.277 | −1.012 | −1.608 | 1019 | 11467200 |
| carbamazepine | 20 | 1.887 | −1.047 | −2.228 | −0.453 | 1.009 | −0.634 | 0.007 | 0.176 | −0.391 | 1019 | 11487941 |
| pentamidine | 11.76 | −2.143 | −4.070 | −3.893 | −0.932 | −1.397 | −1.865 | −2.038 | −1.261 | −3.248 | 1020 | 11467701 |
| pentamidine | 20 | −1.636 | −3.221 | −3.397 | −1.306 | −1.151 | −2.196 | −0.869 | −0.621 | −1.273 | 1020 | 11487971 |
| neriifolin | 20 | −0.034 | 0.392 | −0.435 | −0.351 | −0.062 | 0.711 | −0.798 | −0.822 | −0.562 | 1021 | 11488349 |
| citropten | 20 | −1.279 | 0.097 | −1.290 | −0.080 | 0.342 | 0.140 | −0.358 | −0.533 | 0.064 | 1022 | 11488630 |
| N-methyl-D-aspartic acid | 20 | −0.482 | −0.100 | −0.376 | −0.637 | 0.408 | −0.275 | 0.240 | 0.210 | 0.260 | 1023 | 11489396 |
| dibenzothiophene | 20 | −0.010 | 0.007 | −1.120 | −0.421 | 0.284 | 0.679 | −0.451 | −0.101 | −1.006 | 1024 | 11488827 |
| acetylphenylalanine | 20 | 0.715 | 1.525 | −0.051 | −0.384 | 0.676 | 0.571 | 0.669 | 0.760 | 0.355 | 1026 | 11489168 |
| nalbuphine | 11.2 | −1.178 | −0.542 | −1.290 | −0.208 | 0.441 | −0.613 | 0.285 | 0.696 | −0.637 | 1027 | 11467266 |
| rosolic acid | 20 | −0.618 | 0.479 | −0.312 | −1.145 | −0.025 | 0.187 | −1.477 | −0.286 | −3.643 | 1028 | 11488578 |
| indoprofen | 14.22 | −1.226 | −0.927 | −2.111 | −0.752 | 0.446 | 1.566 | −0.251 | −0.188 | −0.321 | 1029 | 11467984 |
| indoprofen | 20 | −1.164 | −0.920 | −0.622 | −0.662 | 0.768 | 0.319 | 0.056 | 0.035 | 0.068 | 1029 | 11488601 |
| fenoterol | 13.18 | 0.195 | 0.115 | −1.586 | −1.093 | 0.259 | −1.597 | −0.265 | −0.101 | −0.549 | 1033 | 11467430 |
| fenoterol | 20 | 0.062 | 0.922 | −0.379 | −0.300 | 1.191 | −0.262 | 0.071 | −0.044 | 0.293 | 1033 | 11489204 |
| acetylglutamic acid | 20 | −0.950 | −0.372 | −0.807 | 0.004 | 0.942 | −0.444 | 0.310 | 0.166 | 0.560 | 1034 | 11489165 |
| meclozine | 10.24 | 0.016 | 0.594 | −0.273 | −0.139 | 1.378 | −0.039 | −0.711 | −0.322 | −1.358 | 1035 | 11467605 |
| meclizine | 20 | 0.628 | −0.312 | 0.635 | 0.500 | 2.646 | 0.156 | 1.309 | 1.497 | 0.610 | 1035 | 11487926 |
| enalapril | 20 | −0.352 | 0.288 | −0.660 | −0.050 | 0.099 | −0.112 | −0.678 | −0.745 | −0.406 | 1036 | 11489271 |
| cefadroxil | 11 | −0.655 | 0.236 | −1.404 | −0.662 | −0.125 | 0.223 | −0.580 | −0.400 | −0.824 | 1037 | 11467582 |
| cefadroxil | 20 | 0.195 | 1.578 | −1.221 | 0.414 | 0.399 | 0.289 | −0.900 | −0.914 | −0.757 | 1037 | 11487903 |
| oxotremorine | 20 | −1.489 | −1.076 | −0.215 | −0.476 | 1.202 | −0.261 | −0.412 | −0.366 | −0.420 | 1038 | 11489384 |
| eburnamonine | 13.58 | −0.759 | 0.053 | −1.487 | −0.487 | 0.351 | 0.126 | 0.126 | 0.007 | 0.349 | 1039 | 11467755 |
| eburnamonine | 20 | −0.505 | −0.483 | −0.739 | −0.267 | 0.275 | 0.287 | −0.169 | −0.061 | −0.345 | 1039 | 11489320 |
| prochlorperazine | 10.7 | 0.496 | 0.031 | −0.411 | 0.183 | 1.779 | −0.482 | 1.093 | 0.911 | 1.254 | 1041 | 11467547 |
| prochlorperazine | 20 | −0.374 | −0.550 | −1.926 | 1.205 | 1.894 | 0.405 | −0.283 | −0.426 | 0.062 | 1041 | 11489113 |
| merbromin | 5.66 | 1.019 | 1.139 | −1.209 | −2.986 | 4.234 | 21.904 | −0.859 | −0.695 | −1.023 | 1043 | 11467935 |
| merbromin | 20 | 1.957 | 2.538 | 0.388 | −3.495 | 8.568 | 18.194 | −1.088 | −0.783 | −1.454 | 1043 | 11488449 |
| ursodiol | 20 | −0.795 | −0.134 | −2.521 | −0.320 | 1.682 | 0.756 | −0.037 | 0.288 | −0.702 | 1044 | 11488763 |
| flumethasone | 20 | −0.733 | −0.167 | −0.816 | −0.061 | −0.544 | −0.073 | −0.133 | −0.097 | −0.178 | 1045 | 11489261 |
| hecogenin | 20 | 0.954 | 3.010 | −0.948 | 0.106 | 0.155 | 0.715 | −1.032 | −1.077 | −0.713 | 1046 | 11488379 |
| promazine | 14.06 | −0.582 | −0.563 | −2.407 | −0.926 | 1.386 | −1.034 | −0.079 | 0.052 | −0.333 | 1047 | 11467841 |
| promazine | 20 | −1.189 | −0.312 | −0.741 | −0.318 | 0.777 | −0.352 | 0.023 | −0.041 | 0.124 | 1047 | 11488655 |
| enoxacin | 12.48 | −1.019 | 0.405 | −1.038 | −1.177 | 1.209 | 0.386 | −0.315 | −0.250 | −0.377 | 1048 | 11467501 |
| enoxacin | 20 | 0.085 | 0.467 | −0.478 | −0.685 | 0.722 | 0.243 | −0.766 | −0.582 | −0.966 | 1048 | 11489547 |
| chloroacetoxyquinoline | 20 | 0.077 | −0.386 | −0.873 | −0.690 | 0.479 | −0.249 | 0.310 | 0.633 | −0.444 | 1051 | 11488683 |
| hydroquinidine | 20 | −0.694 | −0.213 | 0.138 | 0.079 | 0.248 | −0.272 | 0.120 | 0.020 | 0.300 | 1052 | 11489155 |
| trimethobenzamide | 10.3 | −0.159 | −0.172 | −0.614 | −0.232 | 0.500 | −0.117 | −0.319 | −0.636 | 0.346 | 1053 | 11467228 |
| trimethobenzamide | 20 | −1.465 | 0.148 | −1.238 | −0.636 | −0.028 | −0.050 | −1.401 | −1.261 | −1.429 | 1053 | 11488660 |
| clofoctol | 20 | −0.833 | 1.721 | −1.149 | −2.157 | −1.502 | −0.531 | −0.681 | −0.186 | −1.558 | 1054 | 11489349 |
| nadolol | 12.92 | 0.347 | −0.290 | −0.846 | −0.338 | 1.238 | 0.338 | −0.387 | −0.091 | −0.915 | 1055 | 11467966 |
| nadolol | 20 | −0.201 | 0.540 | −1.549 | −0.518 | 0.763 | 0.868 | −0.452 | −0.280 | −0.714 | 1055 | 11489362 |
| thioguanine | 20 | −0.940 | 0.920 | −2.346 | −0.835 | −0.712 | −1.454 | 0.683 | 0.967 | −0.044 | 1056 | 11488511 |
| procyclidine | 13.92 | −1.239 | −0.211 | −2.028 | −1.028 | 0.256 | 0.610 | −0.716 | −0.771 | −0.473 | 1057 | 11467992 |
| procyclidine | 20 | −0.102 | −0.064 | −0.530 | −1.291 | 1.339 | −0.060 | −0.545 | −0.339 | −0.848 | 1057 | 11489114 |
| danazol | 20 | 0.440 | 0.314 | −1.138 | 0.168 | −0.552 | 1.060 | −0.252 | −0.208 | −0.221 | 1058 | 11488939 |
| doxepin | 20 | −0.220 | −0.287 | −0.903 | −0.646 | 1.002 | −0.095 | 0.233 | 0.237 | 0.142 | 1059 | 11487949 |
| pimethixene | 13.64 | −0.272 | −0.127 | −1.211 | −0.885 | 0.869 | 0.397 | −0.622 | −0.499 | −0.749 | 1060 | 11467442 |
| triamterene | 15.8 | −0.864 | −0.018 | −1.059 | −0.908 | 0.719 | 0.111 | 0.074 | 0.162 | −0.156 | 1061 | 11467182 |
| triamterene | 20 | −0.509 | 0.250 | −1.578 | −0.930 | 0.638 | 0.183 | 0.734 | 0.936 | 0.166 | 1061 | 11488754 |
| methocarbamol | 16.58 | −0.807 | 1.160 | −0.963 | −0.530 | 1.555 | 0.308 | −0.480 | −0.731 | 0.084 | 1062 | 11467332 |
| methocarbamol | 20 | 0.036 | 2.654 | 0.511 | −0.474 | 0.607 | 1.906 | 0.800 | 0.938 | 0.424 | 1062 | 11489088 |
| dobutamine | 20 | −0.481 | 0.409 | −1.565 | −1.494 | −0.972 | −0.220 | −0.633 | −0.642 | −0.487 | 1063 | 11489351 |
| isosorbide | 16.94 | −0.812 | −0.782 | −1.334 | −0.859 | 0.861 | 0.382 | 0.056 | 0.242 | −0.336 | 1064 | 11467862 |
| isosorbide | 20 | 0.058 | −0.265 | −0.920 | −0.657 | 1.447 | 0.448 | 0.109 | 0.156 | 0.066 | 1064 | 11488885 |
| lobeline | 20 | 0.485 | 0.734 | −0.228 | 1.276 | 0.889 | 1.198 | −0.945 | −0.921 | −0.810 | 1065 | 11489177 |
| cefmetazole | 20 | −0.305 | 0.589 | −0.867 | −0.616 | 0.207 | 0.609 | −0.291 | −0.223 | −0.376 | 1067 | 11489278 |
| ranitidine | 12.72 | 0.191 | 0.319 | −1.831 | −0.407 | 0.553 | 0.312 | −1.078 | −0.905 | −1.263 | 1068 | 11467349 |
| ranitidine | 20 | −1.609 | 0.291 | −1.945 | −0.794 | 0.816 | −0.271 | 0.316 | 0.307 | 0.272 | 1068 | 11489241 |
| pergolide | 12.72 | 0.162 | 0.542 | −0.962 | −1.002 | 1.385 | −1.011 | −0.129 | 0.027 | −0.425 | 1069 | 11467443 |
| pergolide | 20 | 0.335 | −0.143 | 0.059 | −0.706 | 0.930 | 0.336 | 0.188 | 0.085 | 0.321 | 1069 | 11489786 |
| hexestrol | 14.8 | −0.712 | −0.564 | −1.841 | −0.476 | 1.177 | −1.006 | −0.246 | −0.198 | −0.291 | 1070 | 11467847 |
| hexestrol | 20 | 1.429 | −0.753 | −2.041 | 0.418 | −0.899 | 0.353 | −0.952 | −0.594 | −1.518 | 1070 | 11488707 |
| progesterone | 12.72 | 0.392 | −0.655 | 0.732 | 0.006 | −0.413 | −0.187 | 0.442 | 0.313 | 0.619 | 1072 | 11467625 |
| alanyl-DL-phenylalanine | 20 | −0.406 | 0.328 | −0.694 | −0.366 | 0.348 | 0.241 | −0.069 | 0.011 | −0.218 | 1073 | 11489163 |
| tropicamide | 14.06 | −0.740 | −0.343 | −0.819 | −0.706 | 1.365 | 1.285 | −0.349 | −0.547 | 0.084 | 1075 | 11467376 |
| tropicamide | 20 | −1.161 | −0.307 | −1.601 | −0.954 | 1.207 | −0.064 | 0.126 | 0.141 | 0.057 | 1075 | 11488752 |
| xylazine | 18.16 | −1.082 | −0.227 | −1.261 | −0.874 | 1.126 | 0.333 | −0.296 | −0.252 | −0.315 | 1076 | 11467746 |
| xylazine | 20 | 0.639 | 0.226 | 0.174 | −0.403 | −0.036 | −0.288 | −0.292 | −0.230 | −0.361 | 1076 | 11489264 |
| minocycline | 8.74 | −0.150 | 1.286 | −0.919 | −0.905 | 1.108 | 0.612 | 0.421 | 0.175 | 0.848 | 1077 | 11467463 |
| minocycline | 20 | −0.640 | 1.387 | −1.103 | 0.117 | 1.514 | 0.075 | 0.074 | 0.097 | 0.089 | 1077 | 11488863 |
| levodopa | 20.28 | −0.654 | 1.146 | −0.708 | −1.811 | 0.543 | 0.483 | 0.265 | 0.400 | −0.096 | 1079 | 11467165 |
| levodopa | 20 | 0.500 | 0.407 | −1.040 | −1.168 | −0.031 | 1.093 | −1.010 | −1.107 | −0.552 | 1079 | 11488312 |
| D-phenylalanine | 20 | −0.947 | −0.684 | −1.086 | −0.184 | 0.641 | −0.427 | −0.024 | 0.155 | −0.371 | 1080 | 11489374 |
| 4-aminoantipyrine | 19.68 | 0.567 | 0.926 | −1.339 | −0.304 | −0.270 | 1.252 | −1.099 | −1.079 | −0.975 | 1081 | 11467329 |
| aminophenazone | 20 | −0.257 | 1.068 | −1.344 | −0.666 | 0.612 | 1.148 | −0.513 | −0.410 | −0.634 | 1081 | 11488750 |
| bromhexine | 20 | 1.674 | 0.710 | −1.434 | 0.026 | −0.198 | 0.816 | −0.463 | −0.160 | −0.994 | 1082 | 11489342 |
| naphazoline | 19.02 | −0.526 | 0.689 | −0.849 | −1.091 | 1.495 | −0.269 | −0.127 | −0.195 | −0.009 | 1083 | 11467194 |
| naphazoline | 20 | −0.110 | 0.869 | −1.192 | −0.530 | 0.644 | −0.476 | −0.316 | −0.175 | −0.480 | 1083 | 11488870 |
| flutamide | 14.48 | −0.432 | −0.194 | 0.389 | 0.034 | 1.704 | 1.332 | −0.645 | −0.689 | −0.471 | 1086 | 11467328 |
| flutamide | 20 | 1.461 | −0.360 | −0.814 | 0.792 | −0.018 | 0.121 | −0.210 | 0.035 | −0.594 | 1086 | 11489017 |
| dichlorophene | 20 | 0.136 | −0.164 | −0.524 | −1.353 | 0.317 | 0.390 | 0.017 | −0.193 | 0.503 | 1087 | 11489030 |
| clomiphene | 20 | 0.533 | 1.376 | 0.169 | −0.381 | 1.174 | 0.025 | −0.767 | −0.538 | −1.009 | 1088 | 11488955 |
| clindamycin | 20 | 0.050 | 0.714 | −0.864 | 0.284 | 0.845 | 0.525 | −0.601 | −0.505 | −0.697 | 1090 | 11488701 |
| edoxudine | 20 | −1.208 | 0.396 | −0.856 | −0.637 | 0.867 | −0.799 | −0.747 | −0.718 | −0.687 | 1091 | 11489776 |
| ampicillin | 11.44 | −1.225 | −0.766 | −1.864 | −0.560 | 0.652 | −0.307 | −1.078 | −1.037 | −0.988 | 1092 | 11467262 |
| ampicillin | 20 | −0.846 | −0.258 | −1.457 | −0.776 | 1.396 | 0.060 | −0.210 | −0.043 | −0.529 | 1092 | 11488585 |
| sulfameter | 14.28 | −0.944 | −0.666 | −1.124 | −0.109 | 0.220 | 0.087 | −0.791 | −0.572 | −1.069 | 1094 | 11467917 |
| sulfameter | 20 | −1.360 | −0.049 | −1.116 | −0.655 | 1.210 | −0.560 | 0.114 | 0.104 | 0.123 | 1094 | 11489244 |
| benserazide | 15.54 | −0.141 | −1.054 | −0.598 | −0.348 | −0.106 | −0.938 | −0.065 | −0.187 | 0.187 | 1095 | 11468086 |
| benserazide | 20 | −2.722 | −3.927 | −2.336 | 0.303 | 1.417 | −0.129 | −1.291 | −1.371 | −0.930 | 1095 | 11487956 |
| carnitine | 20 | 0.421 | 1.410 | 0.245 | 0.847 | 0.548 | 0.664 | −0.017 | −0.007 | −0.094 | 1096 | 11487825 |
| hydrocortisone | 20 | 0.064 | 0.783 | −0.167 | −0.395 | 0.001 | 0.320 | 1.153 | 0.961 | 1.241 | 1098 | 11488128 |
| acexamic acid | 20 | −0.238 | 0.407 | −0.698 | −0.919 | −0.149 | −0.025 | −0.926 | −0.815 | −0.995 | 1099 | 11488669 |
| labetalol | 12.18 | −1.600 | 0.291 | −0.721 | −1.086 | 0.867 | −0.075 | 0.078 | −0.031 | 0.291 | 1100 | 11467425 |
| labetalol | 20 | −0.153 | 0.411 | −0.707 | −1.038 | 0.010 | 0.137 | 0.453 | 0.564 | 0.126 | 1100 | 11488679 |
| budesonide | 20 | −0.107 | −0.165 | −0.867 | −0.542 | 0.279 | −0.004 | −0.318 | −0.285 | −0.277 | 1101 | 11488439 |
| suprofen | 15.36 | −0.084 | 0.426 | −1.379 | −0.949 | 1.315 | −0.859 | −0.408 | −0.147 | −0.861 | 1102 | 11467964 |
| suprofen | 20 | −0.963 | −0.612 | −0.667 | −0.294 | 0.585 | 0.879 | −0.202 | −0.368 | 0.192 | 1102 | 11489246 |
| sodium dehydrocholate | 20 | −0.058 | −0.236 | −0.875 | −0.220 | 0.251 | 0.701 | −0.139 | −0.104 | −0.124 | 1104 | 11488967 |
| hycanthone | 11.22 | −0.738 | −0.114 | −1.626 | −0.151 | 0.610 | −1.292 | 0.860 | 0.837 | 0.741 | 1105 | 11467503 |
| hycanthone | 20 | −1.418 | −0.146 | −1.485 | −0.090 | 0.221 | −0.009 | −0.383 | −0.138 | −0.828 | 1105 | 11488494 |
| flopropione | 20 | −0.260 | 0.869 | −0.475 | −0.473 | 0.270 | 1.173 | −1.405 | −1.255 | −1.449 | 1106 | 11488770 |
| cyclocreatine | 20 | −1.237 | −0.225 | −1.813 | −1.176 | 0.957 | 0.848 | 0.152 | 0.262 | −0.121 | 1107 | 11488741 |
| antipyrine | 21.26 | −0.606 | −0.249 | −0.829 | −0.523 | 1.394 | 0.898 | 0.573 | 0.636 | 0.287 | 1108 | 11467177 |
| antipyrine | 20 | 0.566 | 0.338 | −0.565 | 0.962 | 1.384 | 0.053 | 1.453 | 1.345 | 1.324 | 1108 | 11487904 |
| medroxyprogesterone | 20 | −0.629 | −1.575 | −1.060 | −0.347 | 2.109 | 0.254 | −0.968 | −0.898 | −0.981 | 1110 | 11487963 |
| colistimethate | 20 | −0.769 | 0.698 | −1.244 | −0.613 | 0.817 | −0.163 | 0.945 | 0.860 | 1.011 | 1111 | 11488891 |
| disopyramide | 11.78 | 0.158 | −0.247 | −1.356 | −0.948 | 0.362 | −0.068 | −0.112 | −0.010 | −0.304 | 1112 | 11467414 |
| disopyramide | 20 | 0.221 | 1.221 | −0.037 | −0.536 | −0.395 | 0.407 | −0.403 | −0.197 | −0.679 | 1112 | 11488849 |
| acemetacin | 9.62 | 0.251 | −0.358 | −1.188 | −0.769 | 1.639 | 0.313 | −0.321 | −0.494 | 0.093 | 1113 | 11467444 |
| acemetacin | 20 | 0.535 | −0.630 | −0.427 | −0.722 | 1.088 | −0.409 | −0.016 | 0.038 | −0.059 | 1113 | 11488978 |
| benzthiazide | 9.26 | 0.101 | 0.052 | −1.733 | −0.960 | 1.797 | 1.057 | −0.581 | −0.604 | −0.422 | 1114 | 11467972 |
| benzthiazide | 20 | 0.498 | −0.053 | −0.755 | −0.375 | −0.253 | −0.067 | −0.438 | −0.203 | −0.768 | 1114 | 11488957 |
| norethynodrel | 20 | −0.126 | 0.366 | −0.861 | −0.641 | 0.264 | −0.058 | 0.093 | 0.116 | 0.109 | 1115 | 11488843 |
| mercaptopurine | 20 | 0.125 | −2.053 | −1.336 | 0.609 | 1.142 | −0.431 | −0.061 | −0.159 | 0.098 | 1116 | 11487876 |
| folic acid | 20 | −0.031 | −0.200 | −0.778 | −0.291 | 0.228 | −0.542 | −0.368 | −0.317 | −0.401 | 1117 | 11489275 |
| N-formylmethionylphenylalanine | 20 | 0.205 | 0.383 | 0.154 | −0.371 | −0.333 | 0.092 | 0.487 | 0.607 | 0.225 | 1119 | 11489009 |
| roxarsone | 15.2 | 1.100 | 0.878 | 1.210 | 1.005 | −0.366 | 0.274 | 0.333 | 0.320 | 0.288 | 1120 | 11468118 |
| roxarsone | 20 | −0.711 | 0.274 | −0.453 | −0.478 | 2.262 | 0.874 | 0.835 | 0.763 | 0.865 | 1120 | 11488295 |
| azobenzene | 20 | −0.872 | 1.236 | −0.857 | −1.081 | 0.275 | 0.149 | 0.717 | 0.787 | 0.439 | 1122 | 11489249 |
| meclofenamic acid | 13.5 | −1.379 | −0.490 | −1.303 | −0.965 | 1.148 | 0.208 | −1.220 | −1.337 | −0.776 | 1123 | 11467354 |
| sodium meclofenamate | 20 | 0.317 | −0.210 | −1.445 | 1.124 | 1.308 | −0.596 | 0.128 | 0.230 | −0.033 | 1123 | 11488861 |
| hyoscyamine | 20 | −0.646 | −1.042 | −0.732 | −0.953 | 0.418 | 0.235 | −1.165 | −1.169 | −0.988 | 1124 | 11487928 |
| todralazine | 17.22 | −0.140 | 0.376 | −1.412 | −0.289 | 0.663 | 0.074 | 0.259 | 0.316 | 0.046 | 1125 | 11467219 |
| todralazine | 20 | −0.520 | 1.082 | −1.365 | −0.503 | −0.108 | 0.438 | 0.097 | 0.119 | 0.117 | 1125 | 11488963 |
| phenytoin sodium | 20 | −0.113 | 1.189 | −0.121 | 0.597 | 0.631 | 0.674 | −0.810 | −0.760 | −0.760 | 1126 | 11489097 |
| indapamide | 20 | 0.701 | 0.658 | −0.971 | 0.188 | 0.616 | −0.659 | 0.235 | 0.152 | 0.433 | 1127 | 11488796 |
| piromidic acid | 13.88 | 0.960 | 0.501 | −1.520 | −0.452 | 1.051 | −0.210 | −0.254 | −0.053 | −0.618 | 1129 | 11467953 |
| piromidic acid | 20 | −0.829 | 0.578 | −1.525 | −0.432 | 0.880 | 0.980 | −0.243 | −0.233 | −0.219 | 1129 | 11489281 |
| fluphenazine | 9.14 | 0.095 | −0.168 | 0.102 | 1.160 | 1.059 | −0.074 | 0.802 | 0.639 | 0.989 | 1130 | 11467468 |
| flufenazine | 20 | 1.020 | 0.118 | 1.290 | 0.563 | 0.736 | −0.485 | 0.487 | 0.386 | 0.675 | 1130 | 11489015 |
| hydroflumethiazide | 12.08 | −0.321 | 1.235 | −0.769 | −0.610 | 0.790 | −0.610 | −1.026 | −1.097 | −0.731 | 1131 | 11467161 |
| hydroflumethiazide | 20 | −0.676 | 0.157 | −1.183 | −0.378 | 0.618 | 0.369 | −0.231 | −0.177 | −0.214 | 1131 | 11488826 |
| chlorpropamide | 14.46 | −0.326 | 2.633 | −0.291 | 0.368 | 0.287 | 0.928 | −0.085 | −0.101 | −0.046 | 1132 | 11467471 |
| chlorpropamide | 20 | −0.004 | 0.756 | −0.045 | 0.986 | 1.009 | 1.558 | 0.969 | 0.769 | 1.127 | 1132 | 11487905 |
| lysergol | 15.72 | −0.510 | 0.207 | −1.006 | −1.681 | 0.791 | 0.344 | −0.745 | −0.399 | −1.318 | 1134 | 11467602 |
| mitoxanthrone | 9 | −2.511 | −1.146 | −3.096 | −0.273 | −1.616 | −6.280 | 0.039 | 0.013 | 0.084 | 1135 | 11467533 |
| mitoxanthrone | 20 | −6.150 | −5.230 | −4.730 | −2.820 | −3.148 | −1.242 | 0.121 | −0.779 | 1.953 | 1135 | 11488724 |
| hydrocortisone hemisuccinate | 20 | −0.936 | −0.993 | −1.102 | 0.140 | 0.566 | −0.370 | −0.432 | −0.510 | −0.106 | 1136 | 11489085 |
| diethylstilbestrol | 14.9 | −0.705 | −0.971 | −2.051 | −0.256 | 0.178 | 0.228 | 0.068 | 0.362 | −0.525 | 1138 | 11467904 |
| diethylstilbestrol | 20 | −0.300 | −0.525 | −1.931 | −0.006 | 1.254 | −0.250 | 0.673 | 0.698 | 0.437 | 1138 | 11487964 |
| ethinyl estradiol | 20 | 1.138 | 0.575 | −1.972 | −1.024 | 0.416 | 0.376 | −0.421 | −0.146 | −0.832 | 1139 | 11488820 |
| retinyl acetate | 20 | 1.151 | 0.777 | −0.672 | −0.031 | −0.007 | 0.772 | −1.220 | −1.077 | −1.239 | 1140 | 11488317 |
| benztropine | 20 | −0.529 | −1.577 | −2.046 | 0.055 | 1.455 | −1.170 | −0.332 | −0.183 | −0.624 | 1141 | 11487922 |
| zidovudine | 20 | −1.328 | −0.159 | −1.105 | −0.815 | 0.567 | 0.637 | −0.345 | −0.166 | −0.667 | 1142 | 11488743 |
| fenspiride | 15.36 | −0.076 | 0.370 | −1.797 | −0.502 | 0.830 | −0.076 | −0.481 | −0.472 | −0.448 | 1144 | 11467361 |
| fenspiride | 20 | −1.248 | 0.927 | −1.260 | −0.708 | 0.880 | −0.127 | −0.323 | −0.227 | −0.452 | 1144 | 11489212 |
| sulfanilamide | 23.22 | 1.066 | 1.330 | −0.944 | −0.640 | −0.379 | −0.647 | 0.029 | 0.022 | 0.032 | 1146 | 11467877 |
| sulfanilamide | 20 | −0.872 | 0.421 | −0.772 | −0.667 | 0.416 | 0.532 | −0.397 | −0.187 | −0.773 | 1146 | 11488662 |
| bergapten | 20 | −0.501 | 0.188 | −0.679 | −0.740 | 0.234 | 0.328 | −0.412 | −0.448 | −0.211 | 1147 | 11488350 |
| streptozosin | 20 | 0.049 | −0.382 | −0.932 | −0.257 | 0.042 | 0.717 | 0.137 | 0.024 | 0.271 | 1149 | 11487878 |
| azelaic acid | 20 | −1.214 | −0.024 | −1.754 | −0.890 | 0.433 | −0.125 | −0.501 | −0.394 | −0.554 | 1150 | 11488811 |
| alpha-tochopherol | 20 | 0.471 | 0.587 | −0.595 | 0.447 | 0.323 | 0.544 | −1.489 | −1.206 | −1.789 | 1151 | 11488538 |
| tripelennamine | 20 | −0.871 | 0.257 | −0.283 | −1.094 | 0.920 | 1.663 | −0.992 | −0.897 | −1.015 | 1152 | 11488773 |
| strychnine | 20 | 0.311 | 0.458 | −0.684 | 0.051 | 0.490 | 0.031 | 0.699 | 0.729 | 0.448 | 1154 | 11487893 |
| sulfabenzamide | 14.48 | −0.547 | 0.121 | −0.357 | −0.804 | 1.590 | −0.031 | −0.151 | −0.115 | −0.197 | 1155 | 11467859 |
| sulfabenzamide | 20 | −2.139 | 0.688 | −1.959 | −0.602 | 0.078 | 0.410 | 0.120 | 0.187 | −0.036 | 1155 | 11489133 |
| gentisic acid | 20 | −0.811 | 0.666 | −1.242 | −2.086 | 0.540 | 0.210 | −0.514 | −0.406 | −0.654 | 1156 | 11488589 |
| ketoconazole | 20 | 0.144 | −0.607 | −0.254 | −0.327 | 1.704 | 0.109 | −0.113 | −0.313 | 0.253 | 1157 | 11487946 |
| perhexiline | 14.42 | −0.143 | −0.146 | −1.391 | −0.963 | 1.080 | −4.917 | 0.239 | 0.350 | −0.028 | 1158 | 11467434 |
| perhexiline | 20 | −5.923 | −8.143 | −6.395 | −3.988 | −1.486 | −0.729 | −3.483 | −4.037 | −1.656 | 1158 | 11489355 |
| sulfadiazine | 15.98 | −0.436 | 0.216 | −1.326 | −0.599 | 1.154 | −0.501 | −1.619 | −1.536 | −1.513 | 1159 | 11467171 |
| sulfadiazine | 20 | −1.032 | −0.063 | −0.723 | −0.299 | 1.058 | 0.301 | −0.144 | −0.263 | 0.133 | 1159 | 11489135 |
| nifenazone | 12.98 | −0.606 | −0.220 | −2.019 | −0.474 | 0.633 | −0.977 | −0.862 | −0.909 | −0.626 | 1162 | 11467373 |
| nifenazone | 20 | 0.123 | 0.371 | −0.430 | −0.210 | 1.445 | −0.156 | 0.389 | 0.087 | 0.911 | 1162 | 11488676 |
| naltrexone | 20 | 0.167 | 0.046 | −1.996 | −0.374 | 0.817 | 0.312 | 0.200 | 0.396 | −0.235 | 1163 | 11489363 |
| diethylcarbamazine | 20.08 | −0.682 | 0.321 | −1.825 | −0.747 | 0.102 | 0.200 | −0.219 | −0.196 | −0.230 | 1164 | 11467432 |
| diethylcarbamazine | 20 | −0.556 | 0.154 | −1.272 | −0.352 | 0.059 | 0.308 | 0.586 | 0.706 | 0.297 | 1164 | 11488838 |
| aminocaproic acid | 30.5 | −0.552 | 0.200 | 0.544 | 0.257 | 1.822 | 0.280 | 1.061 | 0.869 | 1.249 | 1167 | 11468108 |
| 6-aminocaproic acid | 20 | −0.572 | −0.949 | −0.672 | −0.732 | 0.474 | −0.680 | −0.523 | −0.316 | −0.897 | 1167 | 11487947 |
| estriol benzyl ether | 20 | 0.551 | 2.168 | 2.975 | 3.459 | −0.504 | 1.667 | −0.114 | 0.122 | −0.574 | 1168 | 11489258 |
| norethindrone | 20 | −0.977 | 0.144 | −1.543 | −0.681 | 1.394 | 0.056 | −0.684 | −0.694 | −0.459 | 1169 | 11488882 |
| aspirin | 20 | −0.717 | −0.668 | −1.128 | −0.523 | 0.336 | −0.047 | −0.390 | −0.241 | −0.579 | 1171 | 11489715 |
| fenofibrate | 11.08 | −1.195 | 0.944 | −1.105 | −0.813 | 0.626 | −0.542 | 0.389 | 0.325 | 0.442 | 1172 | 11467423 |
| fenofibrate | 20 | −0.791 | −0.278 | −1.298 | −0.064 | 0.401 | 0.279 | 0.279 | −0.005 | 0.803 | 1172 | 11489206 |
| imipramine | 14.26 | −0.842 | 0.714 | −1.040 | −0.215 | 0.993 | −0.901 | −0.442 | −0.503 | −0.282 | 1174 | 11467220 |
| imipramine | 20 | −0.222 | 0.162 | −1.230 | −0.603 | 0.879 | −0.672 | 0.134 | −0.025 | 0.505 | 1174 | 11488806 |
| sulfathiazole | 15.66 | −1.131 | 1.534 | −0.404 | −0.815 | 1.828 | 0.611 | 0.172 | 0.419 | −0.398 | 1175 | 11467164 |
| sulfathiazole | 20 | −1.556 | 0.653 | −0.273 | −0.504 | −0.433 | 0.353 | 0.782 | 1.028 | 0.117 | 1175 | 11488657 |
| ethambutol | 20 | −0.578 | 0.056 | −1.099 | −0.971 | 0.914 | −0.012 | −0.623 | −0.462 | −0.753 | 1176 | 11488830 |
| sulfamerazine | 15.14 | −0.911 | −0.008 | −1.697 | −0.719 | 0.577 | −0.754 | 0.024 | 0.182 | −0.301 | 1177 | 11467842 |
| sulfamerazine | 20 | −0.611 | −0.004 | −0.919 | −0.003 | 0.927 | 0.288 | −0.480 | −0.501 | −0.339 | 1177 | 11489136 |
| spiperone | 10.12 | −1.242 | 0.116 | −3.128 | 0.096 | −0.540 | −0.992 | −0.131 | −0.030 | −0.314 | 1180 | 11467436 |
| spiperone | 20 | −1.435 | −0.830 | −2.328 | −0.510 | −1.311 | −1.822 | 0.482 | 0.681 | −0.020 | 1180 | 11489242 |
| oxymetazoline | 15.36 | −0.605 | −0.680 | −1.062 | −0.450 | 0.473 | −0.717 | −1.412 | −1.673 | −0.662 | 1181 | 11467372 |
| oxymetazoline | 20 | −0.861 | 0.087 | −1.251 | −0.378 | 0.564 | −0.757 | −0.552 | −0.540 | −0.382 | 1181 | 11488814 |
| adenosine phosphate | 20 | 1.152 | 0.702 | −1.145 | 0.139 | −0.364 | 0.271 | −0.531 | −0.437 | −0.552 | 1184 | 11489018 |
| dapsone | 16.1 | −1.186 | 0.786 | −0.540 | −0.573 | 0.993 | 0.168 | 0.114 | −0.004 | 0.282 | 1186 | 11467183 |
| dapsone | 20 | 0.099 | 0.024 | −1.085 | −0.470 | 0.366 | 0.139 | −0.832 | −0.698 | −0.877 | 1186 | 11488949 |
| estradiol-3-sulfate | 20 | −0.258 | 0.896 | −0.606 | −0.317 | 0.444 | −0.697 | 0.718 | 0.735 | 0.589 | 1187 | 11488355 |
| furosemide | 12.1 | 0.801 | 0.877 | −0.455 | −0.374 | 1.307 | 0.234 | −0.355 | −0.292 | −0.405 | 1188 | 11467489 |
| furosemide | 20 | −1.396 | 0.768 | −1.510 | −0.787 | 0.313 | 0.342 | 0.198 | 0.309 | 0.011 | 1188 | 11488821 |
| cefoxitin | 9.36 | −0.397 | −0.321 | −2.375 | −0.659 | 0.739 | 0.000 | −0.650 | −0.732 | −0.369 | 1189 | 11467980 |
| cefoxitin | 20 | −1.166 | 0.444 | −1.230 | −1.202 | 0.253 | −0.219 | −0.195 | −0.232 | −0.092 | 1189 | 11488492 |
| hydroxytacrine | 20 | −0.081 | −0.060 | −1.660 | −0.541 | 1.330 | 0.143 | −0.729 | −0.736 | −0.500 | 1190 | 11489031 |
| chloroxine | 20 | −1.392 | −1.831 | −2.014 | 1.120 | −2.934 | −0.800 | −1.089 | −0.461 | −2.181 | 1191 | 11488503 |
| sulfisoxazole | 14.96 | −0.535 | 0.366 | −1.140 | −0.765 | 0.970 | 0.001 | 0.293 | 0.304 | 0.217 | 1192 | 11467482 |
| sulfisoxazole | 20 | −1.895 | 0.384 | −0.835 | −0.543 | −0.095 | 0.649 | 0.103 | 0.239 | −0.187 | 1192 | 11489141 |
| phenacetin | 22.32 | 0.588 | 0.157 | −1.856 | −1.039 | 1.110 | −0.611 | −0.311 | −0.148 | −0.584 | 1193 | 11467681 |
| phenacetin | 20 | 0.665 | 1.310 | 0.760 | −0.255 | 1.491 | 0.005 | 0.811 | 0.833 | 0.564 | 1193 | 11489756 |
| strophanthidin | 20 | 0.028 | 0.227 | −0.828 | −1.126 | 1.582 | 1.100 | −0.232 | −0.222 | −0.231 | 1195 | 11488603 |
| zaprinast | 20 | 0.602 | 1.768 | −1.175 | −0.347 | −0.482 | 0.226 | 0.076 | 0.220 | −0.234 | 1196 | 11489263 |
| azathioprine | 20 | −0.519 | −0.888 | −1.658 | 0.139 | 0.426 | −0.533 | −1.254 | −1.242 | −1.093 | 1198 | 11487884 |
| N-formylmethionyl-leucylphenylalanine | 20 | 0.354 | 2.059 | −1.240 | 0.476 | 1.017 | 0.374 | −0.370 | −0.517 | −0.051 | 1199 | 11487824 |
| tranylcypromine | 20 | −0.673 | −0.231 | 0.085 | 0.032 | 0.093 | −0.318 | 0.008 | −0.018 | 0.015 | 1200 | 11488776 |
| trimethoprim | 13.78 | −1.021 | −0.569 | −0.979 | −0.713 | 1.094 | 0.667 | 0.033 | 0.110 | −0.166 | 1201 | 11467356 |
| trimethoprim | 20 | −0.102 | −0.450 | −0.944 | −0.733 | 1.375 | −0.368 | −0.189 | −0.194 | −0.149 | 1201 | 11488753 |
| galanthamine | 13.92 | −0.383 | 1.321 | −0.377 | −0.543 | 1.301 | −1.112 | −0.338 | −0.310 | −0.312 | 1202 | 11467736 |
| methacycline | 9.04 | 0.323 | 2.538 | −0.031 | −0.517 | 0.261 | 0.020 | 0.913 | 0.781 | 0.984 | 1203 | 11468112 |
| methacycline | 20 | −1.645 | −0.365 | −1.744 | −1.616 | 1.083 | 0.723 | −0.340 | −0.600 | 0.258 | 1203 | 11489214 |
| dihydroergotamine | 20 | 0.293 | 0.585 | −0.604 | 0.372 | 1.157 | −0.494 | 0.008 | −0.021 | 0.143 | 1204 | 11489004 |
| lapachol | 20 | −0.409 | −0.409 | −1.211 | 0.035 | −0.544 | 0.325 | −1.316 | −1.308 | −1.077 | 1205 | 11489267 |
| picrotoxinin | 20 | −1.116 | 0.141 | −1.611 | −1.064 | 1.094 | 0.172 | −0.512 | −0.408 | −0.551 | 1206 | 11488901 |
| chlorpheniramine | 14.56 | −1.458 | 0.060 | −1.540 | −0.945 | 0.521 | 0.080 | 0.504 | 0.533 | 0.308 | 1207 | 11467265 |
| phenylephrine | 20 | −0.349 | −0.455 | 1.105 | −0.460 | 0.474 | −0.187 | 0.254 | 0.120 | 0.536 | 1208 | 11489096 |
| ketoprofen | 15.74 | −0.408 | −0.207 | −0.502 | −0.432 | 1.026 | 1.538 | 0.103 | −0.084 | 0.427 | 1209 | 11467367 |
| ketoprofen | 20 | 0.048 | 0.194 | 0.268 | −0.241 | 0.545 | 0.119 | −0.471 | −0.428 | −0.486 | 1209 | 11488772 |
| probucol | 7.74 | −1.116 | −0.447 | −1.514 | −0.459 | 0.627 | −0.232 | 0.311 | 0.396 | 0.076 | 1210 | 11467532 |
| probucol | 20 | −0.586 | −0.915 | −1.114 | −0.142 | 0.464 | −0.180 | −0.053 | −0.002 | −0.136 | 1210 | 11489216 |
| methoxyamine | 20 | −1.064 | 0.789 | −1.833 | −0.754 | 0.751 | 0.749 | −1.274 | −1.049 | −1.494 | 1211 | 11488730 |
| sulindac | 20 | 0.777 | 0.793 | −0.974 | −0.591 | 0.107 | −0.574 | −0.304 | −0.224 | −0.401 | 1212 | 11489142 |
| betahistine | 29.38 | −0.129 | 0.297 | −0.984 | −0.896 | 0.557 | 0.296 | −0.175 | −0.111 | −0.280 | 1213 | 11467691 |
| betahistine | 20 | 1.068 | 1.977 | 0.861 | 0.125 | −0.176 | 0.386 | −0.284 | −0.008 | −0.718 | 1213 | 11489007 |
| molsidomine | 16.44 | −0.281 | −1.227 | −1.312 | −0.968 | 1.246 | −0.533 | −0.253 | −0.168 | −0.366 | 1214 | 11467695 |
| molsidomine | 20 | −0.665 | −0.269 | −0.651 | −0.537 | −0.398 | 0.339 | 0.250 | 0.331 | 0.106 | 1214 | 11488994 |
| fendiline | 12.68 | 0.636 | −0.301 | 0.307 | 0.421 | 1.303 | −0.405 | 0.614 | 0.636 | 0.448 | 1215 | 11467418 |
| fendiline | 20 | −0.080 | 0.842 | −1.007 | −1.174 | 0.912 | −0.752 | −1.005 | −0.896 | −0.951 | 1215 | 11488822 |
| estriol | 20 | −0.020 | 1.939 | −0.528 | 1.334 | 0.104 | 0.814 | −0.307 | −0.108 | −0.583 | 1216 | 11488779 |
| tetracaine | 15.14 | −0.370 | 0.665 | −0.809 | −0.119 | −0.042 | −0.536 | −0.246 | −0.058 | −0.584 | 1218 | 11467719 |
| tetracaine | 20 | −0.539 | −0.220 | −0.048 | −0.492 | 0.320 | 0.383 | −0.280 | −0.376 | −0.034 | 1218 | 11489144 |
| norgestrel | 20 | −0.966 | 0.593 | −0.761 | −0.965 | 0.831 | −0.027 | −0.225 | −0.183 | −0.187 | 1219 | 11488823 |
| (+)-bicuculline | 10.88 | −0.190 | −0.677 | −1.330 | −0.852 | 0.256 | 0.300 | 0.304 | 0.163 | 0.533 | 1220 | 11467737 |
| cyclopentolate | 13.72 | −0.114 | −0.678 | −0.400 | −0.335 | 0.345 | 0.859 | 0.035 | 0.091 | −0.093 | 1221 | 11468243 |
| cyclopentolate | 20 | −0.300 | 0.293 | −0.756 | −0.072 | −0.406 | −0.283 | 0.556 | 0.830 | −0.037 | 1221 | 11488938 |
| theobromine | 22.2 | −1.067 | −0.710 | −0.948 | −0.513 | 0.512 | 0.007 | −0.141 | −0.294 | 0.207 | 1222 | 11468022 |
| theobromine | 20 | −0.264 | 0.392 | −0.982 | −0.847 | 0.179 | −0.026 | −0.561 | −0.449 | −0.609 | 1222 | 11488801 |
| acebutolol | 11.88 | −0.383 | −0.543 | −0.837 | −1.006 | 1.266 | −0.884 | −0.311 | −0.431 | −0.037 | 1223 | 11467217 |
| acebutolol | 20 | −0.271 | 0.310 | −0.548 | 0.016 | 1.061 | 0.214 | −0.511 | −0.591 | −0.254 | 1223 | 11489156 |
| estradiol cypionate | 20 | 0.219 | 0.452 | −2.428 | −0.489 | −0.010 | 0.408 | −0.124 | 0.072 | −0.418 | 1224 | 11488819 |
| chrysin | 15.74 | 1.243 | 1.238 | 0.370 | 0.765 | −0.602 | 0.920 | 0.205 | 0.280 | 0.003 | 1225 | 11468037 |
| chrysin | 20 | −0.802 | 0.097 | −1.566 | −0.941 | 0.949 | 0.655 | −0.137 | 0.071 | −0.552 | 1225 | 11488569 |
| gamma-aminobutyric acid | 20 | 1.043 | −0.314 | −0.777 | 0.550 | 0.209 | −0.038 | −0.119 | 0.212 | −0.693 | 1227 | 11489024 |
| thimerosal | 20 | −5.590 | −8.416 | −6.206 | −3.315 | −3.880 | −5.732 | 2.733 | −1.197 | 10.413 | 1228 | 11488368 |
| N-acetylneuramic acid | 20 | 1.009 | 0.869 | −0.118 | 0.053 | 0.063 | −0.254 | 1.061 | 1.111 | 0.813 | 1229 | 11488375 |
| N-acetyl-L-leucine | 23.1 | −0.252 | 1.398 | −0.698 | −0.581 | 0.537 | 0.648 | −0.316 | −0.410 | −0.067 | 1231 | 11468044 |
| acetyl-L-leucine | 20 | −0.465 | 0.771 | −0.804 | −0.575 | 1.139 | 0.267 | 0.328 | 0.438 | 0.088 | 1231 | 11488291 |
| tetrahydroxy-1,4-quinone | 23.24 | −1.006 | −0.157 | −1.965 | −0.511 | 0.667 | −0.785 | −0.907 | −0.979 | −0.592 | 1232 | 11467983 |
| tetroquinone | 20 | 0.088 | 0.130 | −0.574 | −0.424 | 0.638 | 0.386 | 0.067 | 0.028 | 0.112 | 1232 | 11488682 |
| peruvoside | 20 | 0.426 | 0.065 | −1.190 | −0.906 | 0.997 | 0.075 | 0.328 | 0.297 | 0.324 | 1233 | 11489218 |
| methylprednisolone | 20 | −0.631 | −0.352 | −1.014 | −0.375 | 0.133 | −0.102 | −0.220 | −0.138 | −0.270 | 1234 | 11489090 |
| chaulmoogric acid, ethyl ester | 20 | −0.015 | −0.123 | −0.612 | −0.536 | 0.513 | 0.617 | −0.730 | −0.876 | −0.265 | 1235 | 11488327 |
| acetaminosalol | 20 | 0.144 | 1.128 | −0.142 | −0.707 | 0.308 | 0.465 | 0.083 | 0.145 | −0.060 | 1237 | 11489247 |
| hexachlorophene | 20 | −3.480 | −5.584 | −1.203 | −2.559 | −3.378 | −2.828 | −2.471 | −1.371 | −4.169 | 1238 | 11488921 |
| dyclonine | 13.82 | −0.872 | 0.108 | −0.221 | −0.746 | 0.775 | 0.597 | −0.237 | −0.137 | −0.405 | 1240 | 11467412 |
| dyclonine | 20 | −0.640 | 0.157 | −0.882 | −1.085 | 0.379 | 1.243 | −0.690 | −0.377 | −1.117 | 1240 | 11488829 |
| sulfaguanidine | 20 | −0.691 | −0.667 | −0.421 | 0.217 | 0.902 | 0.147 | −0.571 | −0.522 | −0.556 | 1241 | 11489236 |
| dipyrone | 12.84 | −0.607 | 0.325 | −1.243 | −0.738 | −0.683 | 0.356 | 0.172 | 0.100 | 0.296 | 1242 | 11467861 |
| dipyrone | 20 | −0.809 | 0.582 | −1.908 | −0.760 | −1.148 | 0.337 | −0.955 | −0.930 | −0.820 | 1242 | 11489369 |
| floxuridine | 20 | −0.712 | 0.511 | −1.643 | −1.275 | 0.063 | −0.314 | 0.909 | 0.993 | 0.539 | 1243 | 11488483 |
| mepenzolate | 11.74 | −0.261 | 1.157 | −0.744 | −0.524 | 1.206 | 0.666 | −0.318 | −0.454 | 0.003 | 1244 | 11467801 |
| mepenzolate | 20 | 0.333 | 0.633 | −0.894 | 1.198 | 0.368 | 0.855 | −0.025 | 0.062 | −0.129 | 1244 | 11488858 |
| pipenzolate | 11.28 | −1.074 | 0.785 | −0.654 | −0.281 | 0.627 | 0.192 | −0.473 | −0.553 | −0.217 | 1246 | 11467908 |
| pipenzolate | 20 | 0.001 | 1.043 | 0.060 | −0.205 | 0.166 | −0.954 | −0.495 | −0.411 | −0.569 | 1246 | 11489330 |
| bithionol | 20 | −2.247 | −4.693 | 0.593 | −2.723 | 1.449 | −2.613 | −1.531 | −1.261 | −1.838 | 1247 | 11487929 |
| estrone hemisuccinate | 20 | −0.076 | 0.242 | −0.686 | −0.573 | 0.834 | 0.250 | 0.261 | 0.370 | −0.003 | 1248 | 11489394 |
| betaine | 20 | 0.548 | −0.215 | 1.036 | 0.345 | −0.348 | −0.998 | 0.478 | 0.354 | 0.604 | 1249 | 11488696 |
| methoxyvone | 20 | 0.596 | −1.793 | −0.620 | 0.104 | 0.512 | 0.419 | −0.499 | −0.706 | −0.030 | 1250 | 11487872 |
| metaproterenol | 18.94 | 0.116 | 0.450 | −1.595 | −1.153 | 0.539 | 0.339 | −0.021 | 0.165 | −0.393 | 1252 | 11467653 |
| metaproterenol | 20 | 0.206 | 0.018 | −0.832 | −1.005 | 2.154 | 0.212 | 0.086 | 0.138 | −0.094 | 1252 | 11487912 |
| citrinin | 20 | −0.040 | 0.637 | −0.980 | −0.590 | 0.122 | 0.012 | 0.608 | 0.730 | 0.218 | 1253 | 11488550 |
| epicatechin | 13.78 | 0.077 | 2.069 | −1.037 | −1.966 | −0.231 | 0.446 | −0.384 | −0.419 | −0.233 | 1254 | 11467790 |
| catechin hydrate | 13.78 | 0.165 | 1.848 | −1.301 | −1.926 | 0.880 | 1.085 | −0.716 | −0.596 | −0.835 | 1254 | 11467965 |
| cianidanol | 20 | 0.630 | 1.283 | −1.471 | −1.746 | −0.371 | 0.639 | −0.113 | −0.357 | 0.347 | 1254 | 11487983 |
| aklomide | 20 | 0.408 | 1.316 | 1.426 | −0.447 | 0.061 | 0.510 | 0.378 | 0.594 | −0.136 | 1255 | 11489327 |
| sulfamethoxazole | 15.8 | 0.057 | 1.529 | −1.373 | −0.894 | 1.190 | 0.332 | −2.872 | −3.540 | −0.968 | 1256 | 11467325 |
| sulfamethoxazole | 20 | −1.184 | 0.305 | −1.108 | −0.666 | 1.114 | 0.962 | −0.491 | −0.629 | −0.140 | 1256 | 11488604 |
| gallamine | 7.84 | 0.266 | −0.114 | −0.712 | −0.287 | 0.302 | 0.036 | 0.626 | 0.747 | 0.207 | 1257 | 11467305 |
| gallamine | 20 | −1.064 | −0.341 | −1.683 | −0.882 | 1.259 | −0.492 | 0.057 | −0.292 | 0.843 | 1257 | 11488894 |
| pipemidic acid | 13.18 | −0.296 | 0.903 | 0.331 | −0.613 | 0.743 | 0.101 | 0.006 | −0.001 | 0.009 | 1258 | 11468045 |
| pipemidic acid | 20 | −0.716 | 0.849 | −0.367 | −0.736 | 0.010 | 0.140 | 0.045 | 0.264 | −0.333 | 1258 | 11489001 |
| pyrimethamine | 16.08 | −0.974 | −0.284 | −1.597 | −1.370 | 3.264 | 1.048 | 0.534 | 0.622 | 0.202 | 1259 | 11467185 |
| pyrimethamine | 20 | 0.994 | 0.449 | −1.810 | −0.696 | 0.329 | −0.306 | −0.657 | −0.526 | −0.825 | 1259 | 11488702 |
| melphalan | 20 | −0.360 | 1.590 | −1.387 | 0.151 | −0.265 | −0.954 | 1.449 | 1.282 | 1.453 | 1260 | 11487890 |
| haloperidol | 10.64 | −0.724 | 0.613 | −1.574 | −0.350 | 0.415 | −0.273 | −0.735 | −0.724 | −0.641 | 1261 | 11467263 |
| haloperidol | 20 | −0.814 | −0.045 | −0.795 | 0.733 | 0.915 | 0.465 | −0.138 | −0.072 | −0.154 | 1261 | 11489084 |
| tranexamic acid | 25.44 | 0.823 | 0.745 | −0.832 | −0.479 | 0.254 | −0.023 | −0.165 | 0.003 | −0.511 | 1262 | 11467319 |
| tranexamic acid | 20 | −0.812 | 0.791 | −0.710 | −0.427 | 0.053 | 0.797 | 0.439 | 0.336 | 0.548 | 1262 | 11488471 |
| artemisinin | 14.16 | 0.529 | −0.511 | −0.505 | −1.117 | 1.177 | 0.522 | 0.282 | 0.354 | 0.087 | 1263 | 11467646 |
| artemisinin | 20 | 0.336 | 0.737 | 0.616 | −0.832 | 0.123 | 1.104 | 0.704 | 0.537 | 0.903 | 1263 | 11489328 |
| salicyl alcohol | 20 | 0.377 | 0.113 | −0.178 | −0.590 | 0.479 | 0.424 | −0.486 | −0.433 | −0.498 | 1264 | 11489127 |
| dicloxacillin sodium salt | 8.5 | −0.036 | 0.270 | −0.883 | −0.055 | 0.390 | 0.836 | −0.206 | 0.009 | −0.612 | 1265 | 11467598 |
| dicloxacillin sodium | 20 | −0.214 | −0.299 | −0.587 | 0.196 | −0.070 | −1.277 | 0.661 | 0.533 | 0.870 | 1265 | 11488797 |
| oxolinic acid | 15.32 | −0.516 | 0.455 | −2.028 | −0.445 | 1.339 | 0.227 | −0.933 | −1.010 | −0.639 | 1266 | 11467341 |
| oxolinic acid | 20 | −0.913 | 0.276 | −1.598 | −0.640 | 0.681 | 0.083 | 0.217 | 0.211 | 0.171 | 1266 | 11488749 |
| acetaminophen | 26.46 | −0.635 | 0.239 | −0.948 | −0.670 | 0.917 | 0.803 | −0.036 | −0.177 | 0.259 | 1267 | 11468016 |
| acetaminophen | 20 | −0.550 | −0.283 | −1.264 | 0.682 | 0.527 | 0.248 | 0.104 | −0.038 | 0.317 | 1267 | 11487901 |
| isoxicam | 11.92 | −0.403 | −0.088 | −1.505 | −0.548 | 1.145 | 0.177 | −0.744 | −0.885 | −0.352 | 1268 | 11467192 |
| isoxicam | 20 | 0.010 | 0.708 | −0.152 | −1.091 | 0.030 | −0.169 | −0.423 | −0.221 | −0.770 | 1268 | 11488678 |
| spaglumic acid | 13.14 | 0.790 | 1.145 | −0.089 | 0.160 | −0.046 | 0.600 | 1.074 | 0.963 | 1.087 | 1269 | 11468239 |
| spaglumic acid | 20 | −0.009 | 0.357 | −0.779 | −0.685 | 0.615 | 0.191 | 0.120 | 0.307 | −0.284 | 1269 | 11489387 |
| hexamethonium bromide | 19.76 | −0.994 | −0.586 | −1.279 | −1.259 | 3.128 | −0.284 | 0.227 | 0.294 | 0.025 | 1270 | 11467186 |
| hexamethonium bromide | 20 | −0.378 | 0.695 | −1.179 | −0.602 | 0.176 | 0.087 | −0.735 | −0.806 | −0.442 | 1270 | 11489368 |
| acetylcarnitine | 20 | −0.980 | 0.681 | −1.275 | −0.930 | 0.476 | −0.192 | −1.051 | −1.015 | −0.943 | 1271 | 11488742 |
| clotrimazole | 11.6 | 0.128 | 0.247 | −0.610 | −1.216 | 2.043 | 0.262 | −0.278 | −0.386 | −0.006 | 1272 | 11467415 |
| clotrimazole | 20 | 0.099 | −1.954 | −2.182 | −1.661 | 2.521 | 0.531 | 0.449 | −0.106 | 1.434 | 1272 | 11487944 |
| prednisone | 20 | −0.474 | 0.376 | −1.054 | 0.258 | 0.253 | −0.034 | −0.290 | −0.177 | −0.473 | 1273 | 11489108 |
| levamisole | 19.58 | 0.266 | 1.515 | −1.556 | −0.266 | 0.798 | −0.129 | −0.076 | −0.060 | −0.144 | 1274 | 11467330 |
| levamisole | 20 | −0.266 | 0.100 | −0.340 | −0.603 | 0.391 | 0.166 | 1.521 | 1.641 | 0.961 | 1274 | 11488681 |
| carbenoxolone | 7 | −0.737 | −0.425 | −1.173 | −0.878 | 1.614 | 0.145 | 0.074 | 0.071 | 0.063 | 1276 | 11467985 |
| lanatoside C | 20 | −0.704 | 0.349 | −0.887 | −1.105 | −0.033 | 0.664 | −0.931 | −0.844 | −0.935 | 1277 | 11489268 |
| diosmin | 20 | −0.712 | 0.452 | −0.708 | −0.501 | 0.321 | −0.433 | −1.223 | −1.150 | −1.147 | 1278 | 11488674 |
| N-(2-aminoethyl)-4-chlorobenzamide | 20 | −1.090 | 0.830 | −0.669 | −1.051 | 1.036 | −0.423 | 0.065 | 0.175 | −0.205 | 1280 | 11489784 |
| chlorothiazide | 13.52 | −0.647 | 1.522 | −0.649 | −0.130 | 0.110 | 0.431 | −0.059 | 0.057 | −0.267 | 1282 | 11467399 |
| chlorothiazide | 20 | 0.116 | 0.445 | −0.711 | −0.831 | 1.202 | 0.566 | −0.331 | −0.608 | 0.221 | 1282 | 11487970 |
| calcein | 20 | 5.567 | 2.057 | −0.243 | −3.437 | 11.668 | 0.465 | −0.625 | −0.468 | −0.829 | 1284 | 11489179 |
| methyl benzethonium chloride | 9.38 | −2.690 | −3.432 | −4.304 | 0.480 | −1.501 | −4.134 | −0.165 | −0.411 | 0.369 | 1286 | 11467853 |
| methylbenzethonium | 20 | −4.580 | −4.939 | −5.614 | −4.202 | −3.108 | −1.750 | −1.868 | −2.103 | −1.043 | 1286 | 11489360 |
| aklavine | 20 | −2.971 | −8.498 | −6.213 | −3.626 | −2.347 | −5.658 | 0.706 | 0.430 | 1.063 | 1288 | 11487895 |
| prednisolone | 20 | 1.018 | 0.134 | 0.031 | 1.121 | 0.248 | −1.111 | −0.042 | −0.155 | 0.191 | 1289 | 11489106 |
| halazone | 20 | −0.242 | −0.116 | −0.445 | −0.334 | 0.779 | −0.942 | 0.565 | 0.593 | 0.385 | 1290 | 11488486 |
| proglumide | 11.96 | 0.138 | −0.867 | −0.599 | −0.312 | 0.068 | −0.001 | −0.513 | −0.787 | 0.094 | 1291 | 11467388 |
| proglumide | 20 | −0.750 | 1.059 | −1.254 | −0.910 | 2.083 | −0.033 | −0.541 | −0.394 | −0.730 | 1291 | 11489222 |
| allopurinol | 20 | 0.376 | 1.218 | −1.611 | −0.204 | 1.448 | −0.105 | −0.869 | −0.849 | −0.798 | 1292 | 11487914 |
| acetylcholine | 20 | −0.299 | −0.013 | −0.712 | −0.520 | 0.622 | −0.038 | 0.358 | 0.533 | 0.014 | 1293 | 11489074 |
| amodiaquine | 20 | −0.547 | −0.270 | −1.008 | −1.178 | 0.877 | −0.008 | 0.398 | 0.317 | 0.481 | 1294 | 11488584 |
| chlorambucil | 13.14 | −0.507 | −0.010 | −0.069 | 0.199 | 0.056 | 0.035 | 0.249 | 0.147 | 0.398 | 1295 | 11468227 |
| chlorambucil | 20 | −0.007 | 0.189 | −1.519 | −0.826 | 0.165 | −0.955 | 0.725 | 0.360 | 1.273 | 1295 | 11487881 |
| eugenol | 20 | −0.943 | 0.081 | −0.243 | −0.816 | 1.068 | 0.307 | −0.254 | −0.166 | −0.320 | 1297 | 11489080 |
| nimesulide | 12.98 | −0.822 | −0.018 | −1.583 | −0.754 | 1.468 | 0.400 | −1.217 | −1.237 | −0.978 | 1298 | 11467342 |
| nimesulide | 20 | 0.614 | 0.567 | −0.534 | −0.731 | 1.174 | 0.089 | −0.661 | −0.775 | −0.313 | 1298 | 11489357 |
| aminohippuric acid | 20.6 | −0.266 | 0.957 | −0.126 | −0.343 | 0.426 | 0.320 | 0.568 | 0.976 | −0.395 | 1299 | 11468043 |
| aminohippuric acid | 20 | 0.597 | 0.226 | −0.983 | −0.606 | −0.310 | 0.243 | 0.220 | 0.160 | 0.304 | 1299 | 11489331 |
| dipyridamole | 7.92 | 2.069 | 1.296 | −0.034 | 0.298 | −0.282 | 0.377 | −0.352 | −0.128 | −0.777 | 1301 | 11467290 |
| dipyridamole | 20 | 1.042 | 0.127 | −0.606 | 0.474 | 0.044 | 0.276 | −0.245 | −0.314 | 0.010 | 1301 | 11488788 |
| bromocriptine | 20 | 1.142 | 0.331 | −1.667 | 0.037 | 0.176 | −0.064 | 0.006 | 0.199 | −0.322 | 1302 | 11488940 |
| clidinium | 11.34 | 0.172 | 0.127 | −2.250 | −0.715 | 1.870 | 0.176 | −0.462 | −0.202 | −0.903 | 1303 | 11467970 |
| clidinium | 20 | −0.084 | −0.723 | −1.393 | −0.772 | 1.625 | −0.062 | −0.556 | −0.503 | −0.602 | 1303 | 11487940 |
| endrin | 20 | 0.522 | 0.305 | −1.710 | 0.129 | 0.770 | 0.726 | −0.680 | −0.738 | −0.385 | 1304 | 11489682 |
| quinine ethyl carbonate | 20 | −0.167 | 0.317 | −0.071 | −0.661 | 0.401 | 0.822 | −1.190 | −1.003 | −1.297 | 1305 | 11489729 |
| mitotane | 20 | −1.631 | 0.341 | −1.708 | −0.542 | 0.460 | −0.216 | −0.030 | 0.072 | −0.244 | 1307 | 11488732 |
| ciclopirox ethanolamine | 19.3 | 0.101 | −1.563 | −2.239 | 2.994 | −1.968 | −0.351 | −0.888 | −0.577 | −1.343 | 1309 | 11467689 |
| ciclopirox olamine | 20 | −1.409 | −1.900 | −3.390 | 2.355 | −1.545 | −0.381 | −0.965 | −1.098 | −0.569 | 1309 | 11487962 |
| sodium beta-nicotinamide adenine dinucleotide phosphate | 20 | −0.562 | 0.550 | −1.286 | −0.582 | −0.038 | 0.101 | −0.424 | −0.307 | −0.615 | 1310 | 11487839 |
| cacodylic acid | 20 | −1.224 | 0.283 | −0.720 | 0.227 | 0.527 | −0.903 | −0.597 | −0.504 | −0.587 | 1312 | 11488986 |
| niclosamide | 12.22 | −1.410 | −6.135 | −3.816 | −3.700 | −3.069 | −3.177 | −0.976 | −1.007 | −0.755 | 1313 | 11467188 |
| niclosamide | 20 | −0.944 | −6.076 | −0.296 | −4.256 | −3.862 | −2.947 | −1.042 | −0.288 | −2.299 | 1313 | 11488999 |
| quinapril | 20 | 0.034 | 0.434 | −1.111 | −0.276 | 1.175 | 0.237 | −0.132 | −0.127 | −0.130 | 1314 | 11488641 |
| hesperidin | 20 | 0.694 | 0.769 | −0.992 | −0.251 | −0.281 | 0.621 | −0.618 | −0.106 | −1.561 | 1315 | 11488619 |
| tulobuterol | 20 | −0.239 | −0.653 | −0.706 | −1.186 | 0.569 | 0.326 | −0.101 | −0.257 | 0.258 | 1316 | 11489432 |
| flutrimazole | 20 | 0.858 | 0.176 | −0.356 | −0.429 | 0.781 | 0.904 | −0.763 | −0.592 | −0.915 | 1317 | 11489422 |
| oxethazaine | 8.56 | −0.497 | −1.284 | −1.374 | −0.742 | 1.092 | −0.550 | 0.473 | 0.335 | 0.609 | 1318 | 11467206 |
| oxethazaine | 20 | −0.294 | −0.217 | −1.451 | 0.173 | −0.055 | −0.278 | −0.739 | −0.812 | −0.486 | 1318 | 11489803 |
| putrescine | 20 | −0.586 | 1.023 | −0.806 | −0.888 | 0.279 | 0.897 | −0.341 | −0.160 | −0.684 | 1319 | 11489751 |
| methylthiouracil | 20 | −0.235 | 0.145 | −0.633 | −0.553 | 0.815 | 0.846 | −0.029 | 0.146 | −0.302 | 1321 | 11489092 |
| scopoletin | 20.82 | 0.413 | 1.951 | 0.261 | 0.146 | −0.017 | 0.501 | −0.462 | −0.513 | −0.291 | 1322 | 11468110 |
| scopoletin | 20 | 0.675 | 2.444 | −0.957 | −0.056 | 0.727 | 0.928 | 0.543 | 0.746 | 0.101 | 1322 | 11489023 |
| ofloxacin | 11.06 | −0.425 | −0.350 | −0.967 | −0.891 | 0.908 | 0.492 | 0.182 | 0.059 | 0.353 | 1323 | 11467385 |
| ofloxacin | 20 | −1.148 | 1.339 | −1.248 | −0.281 | −0.182 | 0.309 | −0.458 | −0.622 | −0.041 | 1323 | 11489280 |
| alexidine | 7.86 | −4.805 | −5.106 | −5.432 | −3.726 | −4.270 | −3.201 | −1.696 | −1.387 | −2.011 | 1324 | 11467925 |
| alexidine | 20 | −2.974 | −1.715 | −2.693 | −1.139 | −0.298 | −3.981 | −0.288 | −0.289 | −0.150 | 1324 | 11488915 |
| cycloleucine | 20 | −1.300 | 0.045 | −1.310 | −0.765 | 0.024 | 0.822 | −0.839 | −0.495 | −1.397 | 1325 | 11488747 |
| 1r-camphor | 20 | −0.744 | 0.270 | −0.402 | −0.794 | 0.042 | 0.372 | 0.073 | 0.107 | 0.023 | 1326 | 11488199 |
| carbachol | 27.18 | −0.239 | −1.509 | 0.905 | −0.296 | 1.223 | −0.202 | −0.060 | −0.176 | 0.191 | 1327 | 11468028 |
| carbachol | 20 | −0.502 | −0.772 | −1.002 | −0.965 | 1.304 | −0.154 | 0.355 | 0.273 | 0.391 | 1327 | 11487930 |
| trichlormethine | 20 | −0.518 | −0.063 | −0.632 | 0.122 | −0.166 | −0.075 | 0.575 | 0.436 | 0.723 | 1330 | 11488596 |
| pentoxifylline | 14.38 | −1.056 | 0.512 | −2.051 | −0.753 | 1.324 | 0.568 | −0.828 | −0.817 | −0.725 | 1331 | 11467344 |
| pentoxifylline | 20 | 0.375 | −0.027 | −0.600 | −0.696 | 0.182 | −0.798 | 0.062 | 0.292 | −0.344 | 1331 | 11488997 |
| chlorthalidone | 11.8 | 0.081 | 0.147 | −1.736 | −0.750 | 1.047 | 0.531 | −0.236 | −0.009 | −0.641 | 1332 | 11467499 |
| chlorthalidone | 20 | 0.308 | 0.323 | −1.134 | −0.257 | 1.655 | 0.066 | 0.793 | 0.646 | 0.877 | 1332 | 11487915 |
| polymyxin b sulfate | 20 | 0.819 | 1.157 | −0.522 | 0.738 | 0.342 | −0.323 | −0.382 | −0.349 | −0.341 | 1333 | 11488306 |
| dexamethasone | 20 | −0.614 | 0.457 | −1.038 | 0.273 | 0.021 | 0.032 | −0.901 | −0.915 | −0.718 | 1334 | 11488640 |
| carbenicillin | 20 | −0.524 | 0.037 | −0.565 | 0.298 | 2.042 | −0.074 | −0.869 | −0.835 | −0.823 | 1336 | 11487936 |
| cloxacillin | 9.18 | −0.639 | −0.258 | −1.268 | −0.547 | 1.697 | 0.381 | −0.901 | −0.799 | −0.974 | 1337 | 11467334 |
| cloxacillin | 20 | −0.395 | 0.231 | −1.769 | −0.339 | 0.259 | 0.459 | −0.568 | −0.506 | −0.508 | 1337 | 11488959 |
| proadifen | 11.32 | −0.388 | −0.746 | −1.220 | −0.557 | 0.616 | 0.223 | −0.364 | −0.414 | −0.201 | 1338 | 11467926 |
| proadifen | 20 | −1.715 | −0.702 | −1.808 | −0.654 | 0.678 | −0.077 | −0.377 | −0.243 | −0.595 | 1338 | 11488740 |
| tiapride | 12.18 | −1.454 | 0.231 | −1.563 | −0.800 | 0.653 | 0.917 | 0.341 | 0.416 | 0.086 | 1339 | 11467364 |
| tiapride | 20 | 1.174 | 0.797 | −0.646 | 0.171 | −0.466 | −0.107 | −0.283 | −0.220 | −0.355 | 1339 | 11489337 |
| triacetin | 20 | −0.842 | −0.008 | −0.717 | −0.571 | 0.431 | −0.264 | −0.327 | −0.187 | −0.569 | 1341 | 11488755 |
| thiram | 20 | −5.812 | −8.121 | −6.222 | −4.011 | −3.915 | −4.853 | −2.420 | −2.770 | −1.220 | 1342 | 11489370 |
| quassin | 20 | −0.688 | 0.866 | −0.400 | 0.033 | 0.925 | −0.233 | 0.095 | 0.205 | −0.168 | 1343 | 11488532 |
| hydrocortisone butyrate | 20 | −1.654 | −0.353 | −0.932 | −0.808 | 1.234 | −0.064 | 0.666 | 0.514 | 0.916 | 1344 | 11488922 |
| mefexamide | 14.26 | −0.541 | −0.271 | −1.363 | −0.719 | 1.172 | 0.129 | −0.340 | −0.498 | 0.009 | 1346 | 11467363 |
| mefexamide | 20 | −2.120 | −0.606 | −1.270 | −0.643 | 0.837 | −0.257 | −0.215 | −0.253 | −0.101 | 1346 | 11489215 |
| fipexide | 10.28 | −0.617 | −0.148 | −0.924 | −0.624 | 0.307 | 0.573 | 0.169 | 0.163 | 0.155 | 1348 | 11467446 |
| fipexide | 20 | 0.721 | −0.397 | −0.186 | −0.354 | 0.873 | −0.071 | −0.241 | −0.010 | −0.657 | 1348 | 11489354 |
| mebendazole | 13.54 | 0.064 | 0.344 | −2.797 | −0.054 | −0.791 | −0.685 | 0.013 | −0.200 | 0.398 | 1350 | 11467365 |
| mebendazole | 20 | 0.512 | −0.483 | −2.060 | −0.434 | −1.003 | −1.473 | 1.160 | 1.212 | 0.824 | 1350 | 11489217 |
| dequalinium | 8.76 | −3.696 | −5.078 | −5.151 | −3.242 | −3.041 | −4.108 | −1.322 | −0.852 | −2.024 | 1351 | 11467536 |
| dequalinium | 20 | −3.176 | −5.120 | −4.446 | −2.506 | −3.055 | −3.241 | −1.733 | −1.087 | −2.705 | 1351 | 11488466 |
| colchicine | 10.02 | −0.197 | −0.451 | −3.253 | −1.055 | −0.345 | −0.642 | 0.551 | 0.807 | −0.072 | 1352 | 11467511 |
| colchicine | 20 | 0.571 | 0.011 | −2.566 | −0.237 | −0.377 | −1.079 | 1.293 | 1.549 | 0.472 | 1352 | 11487891 |
| vulpinic acid | 20 | −1.120 | 0.685 | 0.403 | −3.484 | 1.407 | 1.037 | −0.152 | 0.168 | −0.792 | 1353 | 11488613 |
| picrotin | 20 | −0.278 | 0.205 | −1.155 | −0.167 | 0.917 | 0.184 | 0.253 | 0.404 | −0.167 | 1355 | 11488095 |
| oxyquinoline | 20 | −1.140 | 0.574 | −0.815 | −0.867 | 0.684 | −0.121 | 0.534 | 0.526 | 0.435 | 1356 | 11488513 |
| bupivacaine | 13.86 | −0.663 | −0.329 | −1.440 | −0.938 | 0.545 | −0.058 | −0.227 | −0.316 | 0.002 | 1357 | 11467453 |
| bupivacaine | 20 | −1.202 | −1.058 | −0.841 | 0.084 | 0.801 | 0.190 | 0.392 | 0.548 | 0.009 | 1357 | 11489405 |
| mechlorethamine | 20 | −4.522 | −7.662 | −5.914 | −3.054 | −1.607 | −4.774 | 0.791 | −0.193 | 2.727 | 1358 | 11488264 |
| chlorhexidine | 7.92 | −1.859 | −2.238 | −4.138 | 0.348 | −2.490 | 0.090 | 0.775 | 0.990 | 0.140 | 1360 | 11467291 |
| chlorhexidine | 20 | −0.976 | −1.078 | −2.340 | 0.412 | 1.660 | −1.683 | −0.455 | −0.592 | −0.141 | 1360 | 11487951 |
| methoxy-8-psoralen | 18.5 | 1.463 | −0.070 | −0.371 | 0.605 | 0.103 | 0.227 | 0.803 | 0.952 | 0.340 | 1362 | 11467627 |
| methoxsalen | 20 | 0.416 | 0.776 | −0.976 | −0.034 | 0.310 | −0.889 | −0.562 | −0.513 | −0.489 | 1362 | 11488868 |
| erythromycin ethylsuccinate | 20 | −0.945 | 0.825 | −1.100 | −0.488 | 0.555 | 0.220 | −0.390 | −0.433 | −0.163 | 1363 | 11488799 |
| alpha-cyano-3-hydroxycinnamic acid | 20 | −0.861 | −0.225 | −0.323 | −0.165 | 0.452 | 0.349 | −0.747 | −0.903 | −0.285 | 1364 | 11489287 |
| amitriptyline | 14.42 | −1.086 | −1.116 | −1.566 | −0.712 | 1.177 | −1.338 | −0.898 | −0.914 | −0.743 | 1365 | 11467222 |
| amitriptyline | 20 | −0.040 | −1.393 | −1.785 | 0.136 | −0.471 | 0.013 | −0.903 | −0.890 | −0.807 | 1365 | 11487917 |
| chlorocresol | 20 | −0.414 | −0.105 | −0.607 | 0.528 | 0.102 | 0.747 | 0.206 | 0.611 | −0.728 | 1367 | 11487818 |
| bacitracin | 20 | −0.312 | 0.131 | −0.826 | −0.113 | 1.177 | 0.305 | 1.271 | 1.374 | 0.798 | 1368 | 11488555 |
| dienestrol | 15.02 | 0.829 | −0.794 | −0.652 | −0.205 | −0.210 | 0.894 | 0.033 | −0.191 | 0.481 | 1370 | 11467946 |
| dienestrol | 20 | 0.290 | 0.336 | −0.326 | 0.129 | −0.260 | −0.653 | −0.280 | −0.251 | −0.222 | 1370 | 11488787 |
| altretamine | 19.02 | −0.533 | −0.294 | −0.442 | −0.440 | 0.462 | 0.040 | 0.884 | 0.750 | 0.972 | 1371 | 11468094 |
| altretamine | 20 | −0.315 | 0.427 | −1.416 | −0.322 | 0.103 | −0.631 | 0.379 | 0.488 | 0.069 | 1371 | 11488723 |
| quinolinic acid | 20 | −0.516 | 0.107 | −1.118 | −1.023 | 0.849 | −0.222 | −0.586 | −0.575 | −0.519 | 1372 | 11488745 |
| benzethonium | 9.7 | −3.952 | −4.769 | −4.836 | −0.956 | −1.431 | −3.642 | −0.549 | −0.892 | 0.252 | 1373 | 11467856 |
| benzethonium | 20 | −3.709 | −5.713 | −5.088 | −3.825 | −1.610 | −2.507 | −2.587 | −2.402 | −2.525 | 1373 | 11487950 |
| broxyquinoline | 20 | −2.006 | −1.237 | −1.180 | 1.701 | −1.081 | 0.410 | −0.830 | −1.095 | −0.157 | 1374 | 11488760 |
| penicillin V | 20 | 0.062 | 0.818 | −1.319 | −0.564 | 0.748 | 0.258 | −0.849 | −0.844 | −0.654 | 1377 | 11488311 |
| dopamine | 20 | −0.964 | 0.276 | −0.754 | −0.849 | −0.046 | 0.007 | −0.337 | −0.112 | −0.655 | 1378 | 11488839 |
| potassium p-aminobenzoate | 20 | −0.277 | −0.299 | −1.445 | −0.826 | 0.818 | 0.519 | −1.001 | −1.085 | −0.673 | 1381 | 11487939 |
| salinomycin | 20 | −0.247 | −3.542 | −2.918 | 2.976 | −3.111 | −0.566 | −1.400 | −0.345 | −3.359 | 1383 | 11487889 |
| clopidogrel | 20 | −1.134 | 1.367 | −1.447 | −0.698 | −0.196 | 0.410 | −1.470 | −1.328 | −1.441 | 1384 | 11488328 |
| cinnarazine | 20 | 0.752 | 0.694 | −1.003 | 0.544 | 0.777 | 0.758 | −0.648 | −0.541 | −0.742 | 1385 | 11489347 |
| nomifensin | 16.78 | 0.121 | 1.748 | −0.534 | −0.329 | 3.133 | −1.298 | −0.331 | −0.571 | 0.181 | 1386 | 11467256 |
| nomifensin | 20 | 0.525 | 1.604 | −0.885 | −0.059 | 3.112 | −1.233 | −0.131 | −0.169 | −0.026 | 1386 | 11489364 |
| mefloquine | 10.58 | −0.183 | −0.789 | −1.368 | −0.537 | −0.069 | 0.498 | −0.036 | −0.073 | 0.007 | 1387 | 11467274 |
| mefloquine | 20 | 1.135 | 0.104 | 0.374 | −0.505 | −0.983 | −1.496 | 0.362 | 0.552 | −0.093 | 1387 | 11489332 |
| loratadine | 20 | 0.552 | 0.033 | −1.609 | 0.191 | 0.764 | −0.317 | −0.306 | −0.182 | −0.499 | 1389 | 11489400 |
| clenbuterol | 14.44 | 0.134 | −0.416 | −1.330 | −1.139 | 0.914 | −0.261 | 0.054 | 0.063 | 0.011 | 1390 | 11467493 |
| clomipramine | 12.7 | 0.325 | 0.119 | −0.916 | −0.664 | 0.309 | −1.110 | 0.513 | 0.663 | 0.109 | 1393 | 11467417 |
| clomipramine | 20 | −0.924 | −1.580 | −0.508 | 0.121 | 0.466 | −0.084 | −0.808 | −0.776 | −0.679 | 1393 | 11489545 |
| pipobroman | 20 | −0.792 | −0.161 | −1.907 | −0.580 | 0.342 | 0.441 | −1.074 | −0.918 | −1.219 | 1394 | 11488728 |
| phenoxybenzamine | 13.16 | −0.751 | 0.878 | −0.159 | −0.422 | 0.444 | −0.470 | −0.155 | −0.306 | 0.167 | 1395 | 11468092 |
| phenoxybenzamine | 20 | −0.906 | 0.340 | −1.049 | 0.021 | 0.256 | −0.188 | −1.302 | −1.077 | −1.462 | 1395 | 11489631 |
| chloroxylenol | 20 | −0.639 | −1.134 | −2.046 | −0.917 | 2.165 | 0.499 | −0.811 | −0.857 | −0.614 | 1396 | 11487952 |
| propantheline | 10.86 | −0.361 | 0.808 | −1.670 | −0.893 | 0.589 | −0.615 | −0.301 | 0.104 | −1.055 | 1397 | 11467975 |
| propantheline | 20 | 0.142 | 0.070 | −1.026 | −0.082 | 2.062 | 0.379 | 0.458 | 0.254 | 0.783 | 1397 | 11489116 |
| alpha-tochopheryl acetate | 20 | −0.957 | 0.293 | −1.398 | −0.734 | 0.364 | 0.261 | 0.212 | 0.339 | −0.094 | 1398 | 11488559 |
| ergocalciferol | 20 | 0.372 | −0.913 | −1.658 | −0.449 | 1.541 | −0.118 | 0.661 | 0.723 | 0.357 | 1400 | 11487942 |
| triflupromazine | 11.36 | −0.423 | −0.564 | −1.896 | −0.417 | 1.208 | −0.802 | 0.225 | 0.404 | −0.230 | 1401 | 11467201 |
| edrophonium | 24.06 | 0.250 | 1.406 | 0.901 | −0.161 | 0.343 | 0.177 | −0.803 | −0.611 | −1.083 | 1402 | 11467231 |
| edrophonium | 20 | −0.291 | 0.134 | −0.015 | 0.391 | 1.006 | 1.045 | −0.806 | −1.001 | −0.183 | 1402 | 11488926 |
| arecoline | 25.78 | 0.481 | 1.774 | −0.607 | −0.281 | −1.084 | 0.503 | 0.098 | 0.385 | −0.513 | 1403 | 11467550 |
| arecoline | 20 | 0.018 | −0.361 | −0.452 | −0.451 | −1.032 | 0.839 | −0.545 | −0.261 | −1.072 | 1403 | 11487867 |
| phenazopyridine | 18.76 | −0.413 | −0.676 | −2.094 | 0.126 | 0.587 | 0.678 | −0.561 | −0.323 | −0.930 | 1404 | 11467900 |
| phenazopyridine | 20 | 0.627 | 0.077 | −0.692 | 1.263 | 1.911 | 0.335 | 0.362 | 0.207 | 0.544 | 1404 | 11487833 |
| equilin | 14.9 | −0.882 | −0.045 | −2.270 | −0.408 | 0.259 | −0.069 | −0.626 | −0.538 | −0.676 | 1405 | 11467998 |
| nitromide | 20 | −0.259 | 0.548 | −1.071 | 1.648 | 1.299 | −0.346 | −0.128 | −0.100 | −0.086 | 1406 | 11488860 |
| O-benzyl-L-serine | 20 | −0.140 | 1.374 | 0.176 | −0.374 | 0.228 | 0.513 | 0.750 | 0.854 | 0.378 | 1407 | 11489167 |
| adamantamine | 26.44 | 0.544 | 2.278 | −0.637 | −1.007 | −0.537 | 0.293 | −0.051 | 0.110 | −0.374 | 1408 | 11467555 |
| amantadine | 20 | −0.755 | 0.261 | −0.267 | 1.115 | −0.239 | 0.758 | −0.415 | −0.421 | −0.386 | 1408 | 11487897 |
| carisoprodol | 15.36 | 0.291 | 0.357 | −1.027 | −0.276 | 0.321 | 0.553 | −0.293 | 0.040 | −0.920 | 1409 | 11467571 |
| carisoprodol | 20 | −0.444 | 0.373 | −1.366 | −0.597 | 0.185 | 0.256 | −0.099 | −0.072 | −0.066 | 1409 | 11488969 |
| thiotepa | 20 | −0.029 | 0.586 | −1.879 | 0.010 | 0.678 | −0.181 | −0.262 | −0.289 | −0.156 | 1410 | 11489373 |
| carbinoxamine | 13.76 | 2.471 | 1.444 | −0.685 | −0.435 | −0.603 | 0.034 | −0.720 | −0.795 | −0.435 | 1412 | 11467949 |
| carbinoxamine | 20 | 0.299 | 1.215 | −1.163 | −0.287 | −0.216 | 1.098 | 0.126 | 0.257 | −0.095 | 1412 | 11488943 |
| menthol | 20 | −0.489 | −0.065 | −0.673 | −0.872 | 1.316 | −0.074 | −1.207 | −1.028 | −1.345 | 1413 | 11488671 |
| acetohydroxamic acid | 20 | −0.102 | −0.596 | −1.547 | −0.353 | 1.489 | 0.637 | 0.353 | 0.480 | −0.031 | 1414 | 11487925 |
| N-(3-trifluoromethylphenyl)piperazine | 20 | 0.030 | −0.069 | −1.081 | −0.280 | 0.211 | −0.808 | −0.244 | −0.076 | −0.538 | 1415 | 11489389 |
| 8-cyclopentyltheophylline | 20 | −0.704 | −0.448 | −1.776 | −0.715 | 1.385 | 0.425 | −1.051 | −0.934 | −1.039 | 1416 | 11489550 |
| benzalkonium | 20 | −5.597 | −8.103 | −5.999 | −4.220 | −2.894 | −5.374 | −2.209 | −2.811 | −0.558 | 1417 | 11489382 |
| tetracycline | 9 | −0.461 | 0.284 | 0.314 | −1.031 | 0.987 | −0.461 | 0.538 | −0.066 | 1.642 | 1418 | 11467288 |
| tetracycline | 20 | −0.405 | −0.099 | 0.144 | −0.854 | 0.275 | −0.453 | −0.307 | −0.903 | 0.970 | 1418 | 11489145 |
| cystamine | 20 | 0.077 | 1.203 | 0.470 | −0.542 | 0.154 | 0.575 | −0.241 | −0.229 | −0.232 | 1419 | 11489407 |
| bucladesine | 20 | 0.404 | 0.685 | 0.236 | 0.081 | −0.704 | 0.232 | −0.193 | −0.127 | −0.293 | 1424 | 11489329 |
| mexiletine | 22.32 | −1.055 | 0.883 | −0.564 | 0.161 | 0.003 | 1.114 | −0.622 | −0.495 | −0.746 | 1425 | 11467389 |
| pindolol | 16.1 | 1.151 | 0.582 | 0.527 | −0.282 | 0.171 | 0.446 | −0.179 | −0.067 | −0.414 | 1428 | 11467238 |
| pindolol | 20 | −1.160 | 1.183 | −1.010 | 0.284 | 0.584 | −0.510 | 0.580 | 0.750 | 0.120 | 1428 | 11489101 |
| butamben | 20.7 | 0.465 | −0.329 | −1.413 | −0.405 | 0.458 | −1.345 | 0.249 | 0.462 | −0.220 | 1429 | 11467909 |
| butamben | 20 | −0.599 | −0.280 | −1.083 | 0.155 | 0.587 | 0.499 | −0.168 | −0.299 | 0.111 | 1429 | 11488666 |
| beclomethasone | 20 | −1.221 | 0.138 | −1.585 | −0.124 | −0.235 | 0.742 | −0.363 | −0.121 | −0.717 | 1430 | 11488968 |
| cloperastine | 12.12 | 1.312 | −0.362 | −0.617 | 0.444 | −0.058 | −2.035 | 0.163 | −0.055 | 0.565 | 1431 | 11467941 |
| cloperastine | 20 | −1.911 | −1.783 | −2.310 | 1.329 | −0.584 | 0.236 | 0.098 | 0.023 | 0.244 | 1431 | 11489412 |
| doxylamine | 14.8 | −0.205 | −0.615 | −0.786 | −0.987 | 1.374 | 0.376 | 0.608 | 0.733 | 0.189 | 1432 | 11467175 |
| doxylamine | 20 | −0.540 | −0.432 | −1.702 | −0.607 | 0.824 | 0.403 | 1.206 | 1.249 | 0.832 | 1432 | 11487931 |
| thiamphenicol | 11.22 | −1.067 | 0.539 | −1.142 | −1.450 | 1.069 | −0.244 | 0.379 | −0.128 | 1.288 | 1433 | 11467173 |
| thiamphenicol | 20 | −0.141 | 1.214 | −0.592 | −1.738 | 0.259 | 0.684 | −0.184 | −0.555 | 0.576 | 1433 | 11488672 |
| mianserine | 15.14 | 0.046 | −0.121 | 0.311 | −1.027 | 0.821 | 0.473 | 0.177 | 0.196 | 0.064 | 1434 | 11467247 |
| mianserin | 20 | 1.265 | 2.340 | −0.571 | 0.032 | −0.195 | 0.027 | −2.484 | −2.139 | −2.627 | 1434 | 11489019 |
| prilocaine | 18.16 | −0.849 | 0.292 | −0.942 | −1.028 | 1.264 | 0.451 | −0.114 | −0.250 | 0.147 | 1436 | 11467347 |
| prilocaine | 20 | −0.199 | 0.692 | −1.488 | −0.236 | 1.108 | −0.128 | −0.510 | −0.380 | −0.660 | 1436 | 11489365 |
| busulfan | 20 | −0.872 | −0.512 | −1.724 | −0.640 | 0.750 | −0.091 | −0.521 | −0.589 | −0.351 | 1437 | 11487883 |
| fenoprofen | 16.52 | −1.127 | −0.273 | −2.117 | −0.883 | 0.643 | 0.630 | −0.069 | 0.089 | −0.378 | 1438 | 11467902 |
| fenoprofen | 20 | 0.208 | 0.310 | −0.901 | −0.574 | 0.466 | 0.615 | −0.299 | −0.193 | −0.458 | 1438 | 11489207 |
| methionyl-leucylphenylalanine | 20 | 0.092 | 0.113 | −0.588 | −0.520 | −0.238 | 0.307 | −0.566 | −0.494 | −0.548 | 1439 | 11488338 |
| nabumetone | 17.52 | 1.588 | −0.738 | 0.192 | −0.258 | −0.321 | −0.220 | 0.861 | 0.735 | 0.950 | 1440 | 11468057 |
| nabumetone | 20 | 0.467 | −0.618 | −0.708 | −0.551 | 0.637 | 0.002 | −0.574 | −0.655 | −0.331 | 1440 | 11489785 |
| diphenylpyraline | 14.22 | −0.989 | −0.041 | −1.315 | −0.876 | 1.563 | 0.321 | −0.149 | −0.125 | −0.157 | 1441 | 11467855 |
| diphenylpyraline | 20 | −0.669 | −0.242 | −1.169 | −0.754 | 0.224 | 0.558 | 0.019 | 0.157 | −0.196 | 1441 | 11488798 |
| citiolone | 20 | 0.835 | 1.581 | −0.973 | −0.561 | 0.172 | 0.781 | 0.272 | 0.477 | −0.203 | 1442 | 11489348 |
| orphenadrine | 14.84 | −0.069 | −0.863 | 0.082 | −0.402 | 1.010 | −0.764 | −0.143 | −0.491 | 0.548 | 1443 | 11467387 |
| orphenadrine | 20 | 0.128 | −0.073 | −0.675 | −0.712 | 1.018 | 0.090 | 0.605 | 0.655 | 0.453 | 1443 | 11488854 |
| tetrahydrozoline | 19.98 | −0.895 | −1.172 | −1.638 | −0.912 | 1.093 | −0.635 | −0.209 | −0.275 | −0.038 | 1444 | 11467846 |
| tetrahydrozoline | 20 | −0.613 | −0.208 | −0.804 | −0.172 | 0.334 | 0.250 | 0.144 | 0.168 | 0.073 | 1444 | 11489146 |
| veratrine | 20 | −0.110 | −0.519 | −1.983 | −0.605 | 1.771 | 0.166 | 0.710 | 0.440 | 1.068 | 1445 | 11487954 |
| cevadine | 20 | −0.774 | 1.682 | 0.154 | 0.976 | 0.811 | 0.751 | −0.061 | −0.197 | 0.270 | 1445 | 11488225 |
| cromolyn | 8.54 | 0.234 | 0.485 | −1.485 | −0.558 | 1.343 | −0.227 | −0.015 | 0.163 | −0.379 | 1446 | 11467960 |
| cromolyn | 20 | −0.052 | 0.255 | −0.892 | −0.444 | 0.231 | 0.869 | −0.251 | −0.309 | −0.008 | 1446 | 11488953 |
| salicylamide | 20 | −1.007 | −0.150 | −1.355 | −0.463 | 0.005 | 0.508 | −0.736 | −0.669 | −0.742 | 1447 | 11489128 |
| sulfasalazine | 10.04 | −1.107 | −0.490 | −0.392 | −0.688 | 0.782 | 0.073 | −0.230 | −0.080 | −0.487 | 1448 | 11467668 |
| sulfasalazine | 20 | −0.153 | 1.270 | −1.245 | −0.834 | 1.165 | −0.230 | −0.495 | −0.477 | −0.365 | 1448 | 11489032 |
| (−)-cotinine | 22.7 | 0.390 | 2.001 | −0.864 | −0.172 | −0.622 | 0.628 | 0.395 | 0.540 | −0.026 | 1449 | 11467230 |
| tryptamine | 20 | 0.186 | 1.142 | 0.144 | −0.190 | −0.902 | 0.265 | −0.253 | −0.133 | −0.479 | 1450 | 11488689 |
| demeclocycline | 8.6 | −0.733 | −0.208 | −2.363 | −1.613 | 0.110 | 0.583 | −0.419 | −0.596 | 0.029 | 1451 | 11467901 |
| demeclocycline | 20 | 0.335 | −0.475 | −0.417 | −0.818 | 0.335 | 0.288 | 0.257 | 0.051 | 0.687 | 1451 | 11488948 |
| butacaine | 13.06 | 0.068 | 0.171 | −1.779 | −0.803 | 1.431 | 1.442 | −0.782 | −0.628 | −0.942 | 1452 | 11467979 |
| butacaine | 20 | −0.314 | 1.044 | 1.041 | −0.643 | 0.851 | 0.853 | −0.035 | 0.096 | −0.289 | 1452 | 11489409 |
| morantel | 20 | −0.771 | −0.407 | 0.409 | −0.462 | 0.854 | 0.336 | −0.298 | −0.433 | 0.043 | 1453 | 11489415 |
| digoxin | 20 | −0.496 | 0.763 | −0.712 | −0.649 | 0.811 | 0.445 | 0.055 | 0.352 | −0.478 | 1454 | 11489078 |
| prednisolone acetate | 20 | −0.718 | 0.779 | −0.849 | 0.408 | −0.427 | 0.542 | −0.663 | −0.647 | −0.577 | 1455 | 11489107 |
| amcinonide | 20 | −1.374 | −0.809 | −0.832 | 0.024 | −0.029 | −0.388 | 0.347 | 0.218 | 0.553 | 1457 | 11489404 |
| p-chlorophenylalanine | 20 | −0.693 | −0.481 | −0.682 | 0.132 | 0.459 | −0.836 | −0.912 | −0.932 | −0.693 | 1458 | 11489295 |
| periciazine | 20 | −0.660 | 1.176 | −0.926 | 0.103 | 0.438 | 0.380 | −0.073 | 0.127 | −0.431 | 1459 | 11489420 |
| oxyphencyclimine | 20 | −0.350 | −0.530 | 0.758 | −0.078 | 0.273 | −0.467 | −0.445 | −0.471 | −0.298 | 1461 | 11489416 |
| eucatropine | 20 | −0.370 | 0.786 | −1.051 | −0.460 | 0.779 | −0.178 | −0.784 | −0.829 | −0.468 | 1462 | 11488840 |
| acacetin | 14.08 | −0.644 | −0.392 | −1.686 | −0.249 | 0.407 | 0.284 | −0.125 | −0.153 | −0.031 | 1463 | 11467843 |
| perphenazine | 9.9 | −0.669 | −0.649 | −1.350 | 0.363 | 0.220 | −5.391 | −3.078 | −3.105 | −2.462 | 1465 | 11467273 |
| perphenazine | 20 | −5.616 | −8.464 | −6.728 | −3.919 | −2.181 | −1.097 | −1.879 | −4.321 | 3.415 | 1465 | 11489418 |
| pramoxine | 13.64 | −0.472 | 0.272 | −0.996 | −0.786 | 1.252 | 0.281 | −0.417 | −0.312 | −0.553 | 1467 | 11467864 |
| pramoxine | 20 | 0.281 | −0.351 | 0.408 | 0.784 | 1.645 | 1.272 | −0.384 | −0.464 | −0.213 | 1467 | 11487846 |
| estradiol valerate | 20 | −0.004 | 1.049 | −1.328 | −0.438 | 0.024 | 0.511 | −0.247 | −0.319 | 0.017 | 1468 | 11488789 |
| para-aminoglutethimide | 17.22 | −1.894 | 2.546 | −0.986 | −0.416 | 0.462 | 0.101 | −0.273 | −0.068 | −0.631 | 1469 | 11467392 |
| aminoglutethimide | 20 | −0.567 | 0.702 | −1.626 | −0.414 | 0.643 | 0.832 | −0.932 | −1.079 | −0.505 | 1469 | 11487909 |
| d[-arg-2]kyotorphan acetate | 20 | −0.130 | −0.165 | −0.797 | −0.763 | 0.435 | −0.278 | −0.327 | −0.304 | −0.262 | 1470 | 11488365 |
| chlormezanone | 14.62 | 0.263 | 0.644 | −1.097 | −0.954 | 1.905 | −0.393 | 0.013 | 0.271 | −0.501 | 1471 | 11467484 |
| chlormezanone | 20 | −0.982 | 1.028 | −0.816 | −0.656 | 0.376 | 0.366 | −0.904 | −0.934 | −0.621 | 1471 | 11489623 |
| S-(+)-ibuprofen | 19.4 | −0.372 | 0.087 | 0.383 | −0.423 | 0.394 | 0.056 | −0.090 | −0.188 | 0.114 | 1472 | 11468055 |
| enoxolone | 20 | −2.187 | −0.994 | −1.993 | −1.187 | 0.093 | −1.053 | −0.061 | −0.265 | 0.420 | 1473 | 11488280 |
| cisplatin | 20 | 0.096 | 1.546 | −0.436 | −0.211 | 1.080 | −0.018 | −1.012 | −0.963 | −0.932 | 1475 | 11488715 |
| maprotiline | 14.42 | −0.127 | −0.733 | −2.004 | −0.689 | 0.689 | −3.786 | 0.189 | 0.168 | 0.192 | 1476 | 11467494 |
| maprotiline | 20 | −3.819 | −6.642 | −3.986 | 2.189 | −1.099 | −0.559 | −0.506 | −0.665 | −0.146 | 1476 | 11487955 |
| carboplatin | 20 | 0.649 | 0.654 | −1.486 | 0.094 | 1.451 | 0.594 | 0.053 | 0.165 | −0.217 | 1477 | 11488714 |
| celecoxib | 20 | 0.351 | 0.274 | −1.656 | −0.672 | 0.658 | −0.042 | −0.202 | −0.178 | −0.212 | 1478 | 11489392 |
| (−)-isoproterenol | 18.94 | 0.087 | 0.181 | −0.723 | −1.028 | −0.331 | 0.363 | −0.200 | −0.128 | −0.316 | 1479 | 11468245 |
| isoproterenol | 20 | 0.730 | 0.168 | −0.882 | −0.779 | 0.605 | −0.801 | 0.262 | 0.386 | 0.017 | 1479 | 11488877 |
| chlorzoxazone | 23.58 | 0.774 | 2.443 | −0.611 | −0.272 | −0.121 | −0.145 | −1.678 | −1.598 | −1.554 | 1480 | 11467311 |
| chlorzoxazone | 20 | −0.501 | 0.017 | −0.785 | 0.156 | 0.101 | 0.869 | −0.150 | −0.008 | −0.327 | 1480 | 11488965 |
| dicumarol | 11.9 | 0.172 | −0.207 | 1.467 | −1.176 | 0.459 | 0.667 | 0.083 | 0.097 | 0.040 | 1481 | 11467933 |
| dicumarol | 20 | −0.920 | −0.578 | 0.942 | −1.580 | 1.278 | −0.303 | −0.134 | −0.240 | 0.043 | 1481 | 11487960 |
| hydrastinine | 19.3 | −1.121 | 0.204 | −1.069 | −0.613 | 1.251 | 0.936 | −0.846 | −0.941 | −0.521 | 1482 | 11467343 |
| hydrastinine | 20 | −0.659 | −0.066 | −0.492 | −0.480 | 1.192 | 0.115 | 0.058 | 0.044 | 0.052 | 1482 | 11488558 |
| ethacrynic acid | 13.2 | −0.069 | 1.674 | −0.744 | −0.682 | 0.089 | −0.033 | −0.036 | 0.149 | −0.403 | 1485 | 11467407 |
| ethacrynic acid | 20 | 0.284 | 1.296 | −0.997 | −0.505 | 0.061 | 0.129 | 0.505 | 0.998 | −0.638 | 1485 | 11487913 |
| practolol | 15.02 | 0.251 | 1.735 | −0.117 | −0.473 | 0.732 | 0.038 | −0.600 | −0.494 | −0.698 | 1486 | 11467480 |
| practolol | 20 | −0.031 | 0.132 | −1.098 | −1.018 | 0.234 | 0.424 | 0.087 | 0.203 | −0.164 | 1486 | 11489388 |
| iopanoic acid | 7 | 0.265 | 0.322 | −0.551 | −0.198 | 0.019 | 0.294 | −1.601 | −1.415 | −1.687 | 1487 | 11468200 |
| iopanic acid | 20 | 0.374 | 1.094 | −0.784 | −0.025 | 0.025 | 0.503 | 0.027 | 0.023 | 0.113 | 1487 | 11489022 |
| propafenone | 11.72 | 0.606 | 0.478 | −1.005 | −0.247 | 0.684 | −0.486 | 0.062 | 0.170 | −0.167 | 1489 | 11467647 |
| propafenone | 20 | −0.412 | −1.777 | −1.869 | 0.754 | −0.397 | 0.385 | −0.333 | −0.158 | −0.584 | 1489 | 11489419 |
| clobetasol | 20 | −0.819 | −0.048 | 0.167 | 0.247 | 0.083 | 0.087 | −0.276 | −0.209 | −0.346 | 1493 | 11489410 |
| quipazine | 18.76 | −0.544 | −0.866 | −1.353 | −0.877 | 0.634 | 0.445 | −0.271 | −0.186 | −0.385 | 1494 | 11467765 |
| quipazine | 20 | 0.646 | −1.077 | −0.264 | −0.143 | −0.670 | −0.072 | −0.334 | −0.227 | −0.423 | 1494 | 11488998 |
| thioctic acid | 20 | 0.440 | 1.725 | 0.035 | −0.534 | 0.002 | 0.758 | 0.258 | 0.423 | −0.104 | 1495 | 11489421 |
| methiothepin | 11.22 | −0.990 | −0.955 | −1.614 | −0.409 | 1.188 | −3.974 | −0.262 | −0.159 | −0.427 | 1496 | 11467523 |
| methiothepin | 20 | −2.027 | −5.584 | −3.763 | 1.341 | −0.285 | −1.216 | −0.086 | −0.375 | 0.475 | 1496 | 11489783 |
| foscarnet | 20 | −0.207 | 1.127 | −0.681 | −0.158 | 0.437 | −2.013 | −1.257 | −1.184 | −1.178 | 1498 | 11488484 |
| leflunomide | 14.8 | −0.145 | −0.678 | −0.758 | −0.317 | 0.744 | 0.293 | −0.697 | −0.329 | −1.303 | 1499 | 11467920 |
| tyramine | 20 | −0.048 | 0.109 | −0.760 | −0.170 | 1.977 | 0.316 | −0.493 | −0.679 | −0.038 | 1501 | 11488554 |
| lansoprazole | 10.82 | −0.710 | 0.403 | −0.974 | −0.117 | 0.243 | 0.264 | −1.144 | −1.071 | −1.089 | 1503 | 11468220 |
| lansoprazole | 20 | −0.829 | 0.866 | −1.210 | −0.539 | 0.862 | 0.372 | −0.954 | −0.942 | −0.755 | 1503 | 11488260 |
| buspirone | 10.38 | −0.642 | 0.084 | −0.306 | −0.555 | 1.384 | 0.228 | −0.237 | −0.130 | −0.411 | 1504 | 11467517 |
| isobutylmethylxanthine | 20 | −1.003 | −0.512 | −1.388 | −1.195 | 1.347 | −0.320 | 0.023 | 0.064 | −0.024 | 1505 | 11489551 |
| kojic acid | 20 | 0.022 | −0.307 | −0.583 | −0.698 | 0.452 | −0.170 | −1.176 | −1.166 | −0.914 | 1506 | 11489644 |
| heptaminol | 27.54 | −0.287 | 0.919 | −1.086 | −0.701 | 1.049 | 0.515 | 0.814 | 0.951 | 0.334 | 1507 | 11467163 |
| heptaminol | 20 | −0.057 | 0.764 | −1.067 | −0.723 | 0.571 | −0.049 | −0.838 | −0.743 | −0.880 | 1507 | 11489352 |
| N-formylmethionylalanine | 20 | 0.189 | 0.518 | −1.235 | 0.069 | 1.037 | 0.200 | 0.600 | 0.647 | 0.462 | 1508 | 11488876 |
| ronidazole | 19.98 | −0.232 | −0.358 | −0.118 | 0.050 | −0.309 | −0.232 | 0.352 | 0.460 | 0.051 | 1509 | 11468263 |
| ronidazole | 20 | −0.379 | 1.734 | −0.413 | −0.452 | 1.160 | −0.017 | −0.220 | −0.159 | −0.229 | 1509 | 11488853 |
| methapyrilene | 15.3 | 0.186 | −0.116 | −1.192 | −0.775 | 0.798 | −0.022 | 0.110 | 0.100 | 0.101 | 1510 | 11467490 |
| methapyrilene | 20 | −0.409 | −0.039 | −1.213 | −0.638 | 1.114 | 0.413 | 0.426 | 0.400 | 0.356 | 1510 | 11489802 |
| phenolphthalein | 20 | −0.278 | −1.699 | −2.400 | −1.090 | −0.942 | −2.351 | 0.932 | 0.671 | 1.341 | 1511 | 11489095 |
| pronethalol | 17.44 | −0.005 | 1.102 | −0.566 | −0.600 | 0.951 | −0.239 | −0.038 | 0.090 | −0.309 | 1512 | 11468122 |
| pronetalol | 20 | 0.173 | −0.128 | 0.091 | −0.325 | 0.758 | 0.559 | −0.573 | −0.592 | −0.427 | 1512 | 11489386 |
| benzocaine | 24.22 | −1.335 | −0.085 | −1.492 | −0.788 | 0.998 | 0.490 | 0.227 | 0.141 | 0.358 | 1513 | 11467860 |
| benzocaine | 20 | −0.252 | −0.632 | −2.225 | −0.534 | 1.361 | 0.099 | 0.299 | 0.215 | 0.345 | 1513 | 11487933 |
| fosfomycin | 20 | −0.499 | 0.611 | −1.058 | −0.679 | 0.789 | −0.407 | −0.210 | −0.185 | −0.245 | 1514 | 11488502 |
| tacrine | 20.18 | −0.621 | 1.500 | −0.730 | −0.639 | 0.880 | −0.250 | −0.039 | −0.020 | −0.079 | 1516 | 11467477 |
| aminacrine | 20 | −1.222 | 1.719 | −1.273 | −0.517 | 0.686 | 0.340 | 0.883 | 1.309 | −0.097 | 1516 | 11488928 |
| 9-amino-1,2,3,4-tetrahydroacridine | 20 | 0.348 | −0.633 | −1.045 | −1.032 | 0.240 | 0.967 | −0.946 | −0.736 | −1.154 | 1516 | 11489628 |
| mephenytoin | 18.32 | 0.032 | −0.688 | −0.090 | −0.115 | −0.081 | −0.461 | 0.471 | 0.285 | 0.757 | 1517 | 11468256 |
| diflunisal | 15.98 | −2.269 | −0.477 | −0.998 | −0.194 | 1.159 | 0.255 | −0.541 | −0.378 | −0.797 | 1518 | 11467187 |
| diflunisal | 20 | −0.137 | −0.283 | −1.398 | −0.690 | −0.211 | 0.174 | −0.195 | 0.056 | −0.599 | 1518 | 11488828 |
| dimethadione | 30.98 | −0.300 | 0.012 | −1.204 | −0.494 | 1.532 | −1.458 | −0.735 | −0.571 | −0.929 | 1519 | 11467977 |
| dimethadione | 20 | 0.829 | 0.249 | −0.537 | 0.206 | 0.933 | 0.572 | −0.276 | −0.186 | −0.459 | 1519 | 11487924 |
| hamidium | 20 | −3.449 | −6.380 | −4.647 | −2.831 | −1.903 | −2.061 | −2.145 | −0.555 | −4.967 | 1520 | 11489403 |
| hydroxychloroquine | 20 | −0.578 | −0.057 | −1.295 | −0.692 | 1.276 | −0.214 | 0.033 | −0.008 | 0.194 | 1522 | 11489054 |
| salbutamol | 16.72 | −1.273 | −0.806 | −1.380 | −0.648 | 1.008 | −1.464 | 0.581 | 0.539 | 0.508 | 1523 | 11467346 |
| albuterol | 20 | −0.495 | 0.505 | −0.573 | −0.319 | 0.938 | −0.191 | 0.783 | 0.558 | 1.116 | 1523 | 11489166 |
| isopyrin | 16.3 | 0.412 | 1.307 | −0.704 | −0.743 | −1.749 | 0.254 | 0.093 | 0.153 | −0.053 | 1524 | 11467870 |
| ramifenazone | 20 | −0.444 | 0.456 | −0.091 | −0.668 | −0.730 | 1.006 | 0.468 | 0.538 | 0.230 | 1524 | 11489408 |
| clopamide | 11.56 | −2.152 | 0.299 | −1.129 | −0.726 | 0.878 | −0.184 | −0.075 | 0.110 | −0.440 | 1526 | 11467502 |
| clopamide | 20 | −0.562 | 0.929 | −0.901 | −0.617 | 0.554 | 0.162 | −0.144 | −0.129 | −0.144 | 1526 | 11489411 |
| rotenone | 20 | −2.235 | −4.842 | −4.233 | −2.300 | −0.346 | −2.362 | −1.812 | −1.341 | −2.365 | 1527 | 11488273 |
| mizoribine | 20 | −0.681 | 0.264 | −1.321 | −0.362 | 0.646 | −0.205 | −0.173 | −0.106 | −0.267 | 1528 | 11489375 |
| sulfamonomethoxine | 14.28 | 0.300 | 0.906 | −2.190 | −0.680 | 1.104 | 0.387 | −0.373 | −0.427 | −0.203 | 1529 | 11467971 |
| sulfamonomethoxine | 20 | −1.144 | 0.386 | −0.526 | −0.478 | 0.665 | 0.452 | −0.320 | −0.219 | −0.461 | 1529 | 11489237 |
| harmaline | 18.66 | −0.434 | −0.629 | −0.217 | −0.760 | 1.154 | 1.579 | −0.226 | −0.214 | −0.203 | 1530 | 11467758 |
| harmaline | 20 | 0.790 | 0.791 | −0.838 | 0.032 | 0.388 | −0.615 | −0.533 | −0.659 | −0.125 | 1530 | 11488227 |
| ebselen | 14.58 | −1.470 | 0.244 | −0.380 | −0.959 | −0.844 | −0.439 | −0.938 | −0.999 | −0.627 | 1531 | 11467888 |
| ebselen | 20 | −0.617 | 1.192 | −0.905 | −0.192 | −4.078 | −1.821 | −2.264 | −3.031 | −0.239 | 1531 | 11489257 |
| zomepirac | 13.72 | −0.137 | 0.596 | −1.027 | −0.815 | 0.497 | 0.878 | −0.181 | −0.162 | −0.186 | 1533 | 11467927 |
| zomepirac | 20 | −0.791 | 0.928 | −0.589 | −0.794 | 0.402 | −0.581 | 0.342 | 0.552 | −0.179 | 1533 | 11488771 |
| piperine | 14.02 | 1.753 | −0.553 | −0.741 | −0.378 | −0.318 | −0.301 | 0.020 | −0.005 | 0.060 | 1534 | 11467622 |
| piperine | 20 | 0.565 | −0.406 | −1.093 | −0.009 | 0.688 | 0.269 | −0.944 | −1.088 | −0.526 | 1534 | 11487865 |
| midodrine | 15.74 | −0.603 | 0.608 | −1.667 | −0.627 | 0.636 | 0.698 | −0.629 | −0.303 | −1.198 | 1535 | 11467339 |
| midodrine | 20 | −0.509 | 0.297 | −0.795 | −0.346 | 0.666 | 0.184 | −0.628 | −0.512 | −0.732 | 1535 | 11489361 |
| p-fluorophenylalanine | 20 | 0.072 | 1.220 | −1.136 | −0.627 | 0.637 | 0.280 | −0.118 | 0.011 | −0.390 | 1537 | 11488718 |
| morin | 20 | −0.962 | 1.309 | −0.293 | −2.534 | 0.880 | −0.180 | −0.557 | −0.445 | −0.685 | 1538 | 11488531 |
| monocrotaline | 12.3 | −0.074 | 0.717 | −2.328 | −1.116 | 0.234 | 0.515 | −0.758 | −0.506 | −1.109 | 1539 | 11467751 |
| monocrotaline | 20 | −0.637 | 0.277 | −1.519 | −0.263 | 1.636 | 0.283 | −1.124 | −1.043 | −1.085 | 1539 | 11488722 |
| thiamylal | 20 | 0.131 | 0.166 | −0.754 | −0.672 | 0.362 | 0.190 | −0.453 | −0.487 | −0.252 | 1570 | 11488200 |
| pentobarbital | 20 | 0.149 | 1.143 | −0.468 | −0.137 | −0.496 | 0.095 | 0.061 | 0.008 | 0.101 | 1572 | 11489807 |
| thiopental | 20 | −0.167 | −0.081 | −1.043 | −0.426 | 0.356 | −0.299 | 0.646 | 0.672 | 0.427 | 1573 | 11489814 |
| chlordiazepoxide | 20 | −1.397 | 0.117 | −1.872 | −0.837 | 0.465 | 0.132 | −0.015 | 0.215 | −0.409 | 1575 | 11488973 |
| pomiferin | 20 | −2.196 | −3.179 | −3.847 | −1.730 | −0.899 | −2.458 | −0.984 | −0.931 | −0.914 | 1576 | 11488615 |
| dimercaptopropanol | 20 | 0.558 | 0.652 | −2.089 | 0.155 | 0.556 | 0.282 | −0.488 | −0.408 | −0.474 | 1577 | 11489034 |
| harmalol | 19.98 | 0.225 | −0.390 | −1.308 | −0.761 | −0.130 | 8.863 | −0.142 | −0.065 | −0.282 | 1578 | 11467759 |
| harmalol | 20 | 0.188 | 0.143 | −0.505 | −0.800 | −0.419 | 6.615 | 0.839 | 0.778 | 0.846 | 1578 | 11488372 |
| Ng-methyl-L-arginine acetate | 20 | −0.533 | 0.021 | −0.692 | −0.870 | −0.128 | 0.182 | −0.355 | −0.422 | −0.156 | 1581 | 11489298 |
| beta-propiolactone | 20 | 0.044 | 0.942 | −0.834 | −0.403 | 0.386 | 0.121 | −0.590 | −0.642 | −0.415 | 1582 | 11489798 |
| rhapontin | 20 | −0.359 | 0.348 | −1.009 | −0.981 | 0.073 | 0.179 | 0.675 | 0.675 | 0.559 | 1583 | 11489321 |
| guaiazulene | 20 | −0.274 | 0.237 | −1.147 | −0.886 | 1.360 | −0.055 | −0.067 | −0.071 | 0.029 | 1585 | 11488905 |
| spermidine | 20 | 0.047 | 0.821 | 0.897 | −0.323 | −0.154 | 0.029 | −0.507 | −0.455 | −0.539 | 1586 | 11488690 |
| lividomycin | 20 | −1.398 | −0.063 | −1.482 | −0.929 | 0.368 | 0.116 | −0.417 | −0.533 | −0.026 | 1587 | 11488970 |
| usnic acid | 20 | −0.677 | 0.145 | −2.122 | −0.863 | 0.866 | 0.479 | −0.671 | −0.371 | −1.189 | 1588 | 11488560 |
| leucine enkephalin | 20 | 0.081 | 1.105 | −0.846 | −0.601 | −0.107 | −0.167 | −0.451 | −0.320 | −0.547 | 1589 | 11488993 |
| terfenadine | 8.48 | −0.199 | −0.604 | −1.333 | −0.174 | 0.266 | −0.611 | −0.299 | −0.358 | −0.157 | 1590 | 11467286 |
| N-(9-fluorenylmethoxycarbonyl)-L-leucine | 20 | 0.170 | 1.110 | −0.823 | 0.228 | 1.863 | −1.594 | −0.510 | −0.624 | −0.172 | 1591 | 11489284 |
| N-(g)-nitro-L-arginine | 20 | −1.111 | −0.073 | −0.921 | −0.297 | 0.542 | −0.969 | −0.300 | −0.203 | −0.429 | 1594 | 11489294 |
| gambogic acid | 20 | −5.274 | −8.275 | −6.255 | −3.614 | −3.708 | −5.807 | ND | ND | ND | 1597 | 11488204 |
| safrole | 20 | −1.569 | 0.106 | −1.653 | −0.743 | 1.151 | 0.308 | 0.319 | 0.366 | 0.150 | 1599 | 11488591 |
| actinonin | 20 | −0.831 | −0.270 | −1.219 | −0.829 | 1.177 | 0.354 | 0.189 | −0.113 | 0.830 | 1600 | 11488444 |
| pimpinellin | 20 | −0.369 | 1.040 | −0.698 | −0.031 | 1.874 | −0.988 | 0.229 | 0.264 | 0.086 | 1601 | 11488536 |
| biochanin A | 20 | −0.454 | −0.069 | −1.052 | −0.316 | 0.948 | −0.017 | 0.715 | 0.513 | 0.928 | 1602 | 11487863 |
| succinylsulfathiazole | 11.26 | −0.619 | −0.325 | −1.459 | −0.741 | 0.734 | 0.482 | 0.114 | 0.018 | 0.279 | 1603 | 11467850 |
| succinylsulfathiazole | 20 | −0.607 | 0.111 | −0.718 | −0.673 | 0.617 | 0.367 | −0.361 | −0.409 | −0.239 | 1603 | 11489762 |
| phthalylsulfathiazole | 9.92 | −0.354 | −0.359 | −1.114 | −0.293 | 0.995 | 0.373 | −0.443 | −0.394 | −0.451 | 1604 | 11468017 |
| fluconazole | 20 | 0.018 | 1.381 | 0.188 | −0.480 | 0.167 | 0.741 | −0.329 | −0.543 | 0.216 | 1605 | 11489423 |
| althiazide | 10.42 | 0.474 | 1.673 | −0.458 | −0.163 | −0.976 | 0.491 | −0.293 | −0.138 | −0.554 | 1606 | 11467869 |
| althiazide | 20 | 0.218 | 2.149 | −0.724 | 0.272 | 1.286 | 1.305 | −0.320 | −0.202 | −0.491 | 1606 | 11489183 |
| lovastatin | 9.88 | −1.137 | −2.217 | −2.530 | −0.122 | −1.374 | −1.628 | 0.334 | 0.280 | 0.382 | 1607 | 11467664 |
| lisinopril | 9.86 | 0.378 | 0.387 | −0.230 | −0.202 | 0.395 | 0.265 | −1.562 | −1.629 | −1.109 | 1608 | 11467449 |
| lisinopril | 20 | −0.611 | 0.030 | −1.223 | −0.397 | 0.590 | 0.412 | −0.763 | −0.837 | −0.472 | 1608 | 11489272 |
| gedunin | 20 | 1.017 | −0.064 | 0.444 | 1.552 | −1.448 | 0.455 | −0.592 | −0.374 | −0.990 | 1609 | 11488050 |
| hesperetin | 13.24 | −0.860 | −0.368 | −1.684 | −0.633 | 0.674 | 0.367 | −1.018 | −1.000 | −0.900 | 1610 | 11467272 |
| hesperetin | 20 | −0.764 | 0.123 | −1.157 | −0.650 | 0.147 | −0.348 | −0.680 | −0.463 | −0.959 | 1610 | 11489609 |
| glimepiride | 8.16 | 0.927 | 0.600 | −1.071 | −0.088 | 0.764 | 1.009 | 0.180 | 0.350 | −0.210 | 1624 | 11467799 |
| irbesartan | 20 | 1.023 | 0.581 | −1.038 | −0.454 | 1.092 | −0.331 | −0.652 | −0.729 | −0.328 | 1635 | 11489491 |
| milrinone | 18.94 | 0.265 | 1.049 | −0.224 | −0.030 | 0.704 | 0.140 | −0.026 | −0.046 | 0.005 | 1666 | 11468213 |
| ganciclovir | 15.68 | −0.790 | 0.414 | −1.421 | −0.148 | 0.593 | 0.005 | −0.452 | −0.231 | −0.809 | 1670 | 11467987 |
| oxaprozin | 13.64 | 0.551 | 0.385 | 0.442 | 0.325 | −0.224 | −0.071 | −0.486 | −0.537 | −0.301 | 1672 | 11468208 |
| oxaprozin | 20 | −0.176 | 0.847 | −0.958 | −0.993 | 1.017 | −0.994 | 0.297 | 0.381 | 0.102 | 1672 | 11489512 |
| propofol | 22.44 | 0.480 | −0.249 | 0.018 | 0.039 | −0.419 | 0.242 | 0.262 | 0.387 | −0.047 | 1677 | 11468079 |
| raloxifene | 8.44 | 2.037 | −0.803 | −0.189 | 0.287 | 1.547 | 0.232 | −0.639 | −0.610 | −0.575 | 1694 | 11468010 |
| famciclovir | 20 | −1.094 | −0.720 | −0.988 | −0.488 | 0.599 | 0.779 | −0.135 | 0.125 | −0.573 | 1696 | 11488917 |
| letrozole | 14.02 | −1.150 | −0.488 | −1.505 | −0.128 | 0.554 | −0.105 | 0.806 | 0.656 | 0.942 | 1698 | 11468173 |
| metformin | 30.96 | −0.388 | 1.857 | −0.276 | 0.175 | 0.413 | 0.597 | 0.297 | 0.516 | −0.263 | 1714 | 11467152 |
| fluvastatin | 9.72 | −1.351 | −3.178 | −3.244 | 0.811 | −1.812 | −2.180 | −0.209 | −0.217 | −0.148 | 1736 | 11468007 |
| gabapentin | 23.36 | 0.021 | 0.087 | −0.449 | −1.028 | 0.336 | −0.111 | −0.193 | 0.105 | −0.747 | 1764 | 11468009 |
| nilutamide | 12.6 | −0.653 | −0.364 | 0.031 | −0.324 | 0.641 | −0.135 | −0.933 | −0.970 | −0.685 | 1765 | 11468076 |
| nilutamide | 20 | −0.193 | 0.303 | −1.291 | −0.670 | 0.649 | −0.409 | −1.586 | −1.507 | −1.482 | 1765 | 11489789 |
| mesalamine | 26.12 | −0.213 | 0.208 | −0.560 | −0.257 | −0.577 | −0.325 | −0.451 | −0.499 | −0.270 | 1778 | 11468217 |
| moxonidine | 16.56 | −1.463 | −0.492 | −0.691 | −0.724 | 2.064 | 0.163 | −0.167 | −0.013 | −0.459 | 1779 | 11468164 |
| omeprazole | 11.58 | 0.321 | 0.461 | −1.418 | −0.768 | 0.355 | 0.828 | −0.681 | −0.500 | −0.914 | 1782 | 11467641 |
| modafinil | 20 | −1.484 | −0.436 | −0.859 | −0.387 | 0.644 | 0.141 | −0.907 | −0.764 | −0.985 | 1788 | 11489528 |
| risperidone | 9.74 | −1.004 | −0.813 | −0.267 | 0.478 | 1.159 | 0.243 | 0.887 | 0.615 | 1.266 | 1795 | 11468177 |
| ticlopidine | 15.16 | −0.687 | 0.326 | −1.403 | −0.863 | 1.252 | −0.316 | 0.200 | 0.254 | 0.017 | 1821 | 11467195 |
| dorzolamide | 12.32 | −0.030 | −0.808 | −0.323 | 0.180 | −0.044 | −0.076 | 0.990 | 0.975 | 0.829 | 1829 | 11468264 |
| sildenafil | 20 | −0.402 | −0.645 | −0.519 | 0.312 | 1.028 | −0.864 | −0.496 | −0.551 | −0.254 | 1835 | 11489464 |
| rofecoxib | 20 | −0.930 | 0.935 | −2.253 | −1.000 | 1.755 | 0.131 | 0.209 | 0.138 | 0.363 | 1837 | 11488262 |
| epigallocatechin-3-monogallate | 20 | 1.161 | 1.521 | −0.164 | −0.586 | 0.177 | 0.290 | −0.083 | −0.109 | −0.079 | 1859 | 11487984 |
| MY-5445 | 20 | −0.255 | 0.348 | −0.285 | −0.571 | 1.358 | −1.229 | 0.164 | 0.193 | 0.113 | 1865 | 11489636 |
| bovinocidin | 20 | 0.168 | 0.970 | 0.453 | −0.385 | 0.357 | −0.045 | −0.611 | −0.436 | −0.867 | 1898 | 11488692 |
| flucytosine | 30.98 | −0.729 | 0.283 | −0.714 | −0.503 | 0.520 | 0.107 | −0.687 | −0.685 | −0.567 | 1910 | 11468082 |
| 7-nitroindazole | 20 | −0.990 | 0.589 | −1.157 | −0.933 | 0.565 | 0.618 | −0.238 | −0.178 | −0.276 | 1912 | 11489531 |
| aminocyclopropanecarboxylic acid | 20 | −0.938 | 0.420 | −1.980 | −0.372 | −0.055 | 0.213 | −0.566 | −0.573 | −0.433 | 1923 | 11489291 |
| baicalein | 20 | −0.456 | 1.556 | −1.632 | −2.552 | 0.851 | 1.049 | 0.699 | 0.444 | 1.140 | 1950 | 11488282 |
| betulinic acid | 8.76 | 0.910 | 1.086 | −2.668 | −1.366 | −0.038 | 0.751 | −0.037 | 0.128 | −0.370 | 1960 | 11467565 |
| caffeic acid | 22.2 | −0.291 | 0.443 | −0.963 | −1.448 | −1.104 | 0.512 | −0.136 | −0.192 | −0.004 | 1978 | 11468050 |
| caffeic acid | 20 | −1.020 | −0.153 | −1.817 | −4.427 | −2.045 | −0.003 | −0.914 | −0.783 | −0.966 | 1978 | 11489428 |
| clioquinol | 13.1 | −1.660 | −1.663 | −1.672 | 2.483 | −1.879 | 1.216 | −0.761 | −0.811 | −0.536 | 1999 | 11468034 |
| pentetic acid | 10.16 | 0.590 | −0.276 | 0.586 | 0.181 | −0.104 | 0.444 | 0.509 | 0.395 | 0.633 | 2030 | 11468089 |
| disulfiram | 13.48 | −3.107 | 0.167 | −1.891 | −2.228 | −0.898 | −5.825 | −1.156 | −1.020 | −1.252 | 2038 | 11467245 |
| disulfiram | 20 | −5.476 | −6.037 | −5.086 | −3.244 | −3.188 | −1.545 | −2.620 | −4.097 | 0.927 | 2038 | 11488992 |
| thiorphan | 15.8 | 0.169 | 0.495 | −1.618 | −0.273 | −0.185 | 0.083 | −0.070 | −0.004 | −0.186 | 2041 | 11467781 |
| ellipticine | 16.24 | −4.355 | −6.206 | −3.881 | −1.483 | 1.606 | −5.622 | −1.053 | −0.425 | −2.134 | 2057 | 11467762 |
| formononetin | 20 | 0.658 | 0.203 | 0.503 | 0.495 | 0.115 | −0.178 | 0.656 | 0.471 | 0.947 | 2070 | 11488376 |
| fusaric acid | 22.32 | −0.549 | −0.122 | −1.387 | −0.832 | 0.347 | −0.004 | −0.077 | −0.052 | −0.117 | 2078 | 11467590 |
| gabexate | 12.44 | −0.955 | −0.212 | −0.701 | −0.488 | 1.154 | −0.161 | 0.540 | 0.319 | 0.884 | 2080 | 11468156 |
| miltefosine | 20 | −0.727 | 0.150 | −1.088 | −0.429 | 0.493 | −0.928 | 0.221 | 0.221 | 0.164 | 2097 | 11488495 |
| hydroquinone | 20 | −5.481 | −8.318 | −6.520 | −4.469 | −4.148 | −4.999 | −3.360 | −3.940 | −1.450 | 2101 | 11489488 |
| indole-3-carbinol | 20 | −0.133 | 0.851 | −0.881 | −1.071 | 1.260 | 0.096 | 0.188 | 0.243 | 0.074 | 2109 | 11489526 |
| kaempferol | 13.98 | −0.013 | 0.060 | −0.352 | −2.485 | −1.322 | −0.523 | −0.278 | −0.322 | −0.141 | 2121 | 11468246 |
| luteolin | 13.98 | −0.627 | −0.637 | −0.209 | −1.473 | 0.247 | −0.440 | −0.240 | −0.379 | 0.091 | 2137 | 11468018 |
| myricetin | 12.56 | −0.099 | −0.306 | −1.209 | −1.577 | 0.077 | 0.084 | −0.006 | −0.018 | 0.019 | 2181 | 11467613 |
| clorgyline | 14.7 | −0.489 | 0.494 | −0.460 | −0.631 | 1.023 | 0.228 | 0.051 | 0.338 | −0.541 | 2203 | 11467492 |
| picotamide | 10.62 | −1.774 | −0.210 | −0.155 | −0.285 | 0.828 | −0.504 | −1.237 | −1.260 | −0.986 | 2241 | 11467267 |
| piribedil | 13.4 | −0.355 | 0.175 | 0.905 | 0.249 | 0.639 | 0.264 | 0.451 | 0.552 | 0.153 | 2245 | 11468128 |
| resveratrol | 17.52 | 0.069 | 1.364 | −0.765 | −0.462 | 1.992 | −0.119 | 0.063 | 0.159 | −0.147 | 2269 | 11467656 |
| resveratrol | 20 | −0.963 | 0.437 | −1.562 | −3.524 | −0.339 | −1.039 | −1.233 | −1.262 | −0.925 | 2269 | 11489313 |
| selegiline | 21.36 | −0.251 | 0.099 | −0.278 | −0.594 | −0.032 | 0.224 | 0.124 | 0.155 | 0.035 | 2284 | 11467700 |
| S-nitroso-N-acetylpenicillamine | 20 | −0.254 | 0.568 | −0.843 | −0.321 | 0.213 | 0.155 | −0.292 | −0.237 | −0.342 | 2294 | 11489282 |
| tetrahydropalmatine | 20 | 0.104 | 0.597 | −0.758 | −0.961 | 1.124 | 0.035 | 0.687 | 0.644 | 0.625 | 2321 | 11488552 |
| D,L-threo-3-hydroxyaspartic acid | 20 | −0.438 | −0.580 | −0.811 | −0.716 | 0.366 | 0.934 | −0.297 | −0.299 | −0.195 | 2325 | 11489730 |
| tranilast | 20 | 0.160 | −0.384 | −0.710 | −0.037 | 0.080 | 0.755 | −0.955 | −0.825 | −1.088 | 2335 | 11487858 |
| vinpocetine | 11.42 | 1.168 | 1.960 | 0.886 | 0.174 | 1.353 | 0.297 | 0.092 | 0.142 | −0.024 | 2359 | 11467416 |
| vinpocetine | 20 | 0.669 | 0.260 | −1.030 | 0.242 | 1.318 | −1.089 | −0.370 | −0.400 | −0.250 | 2359 | 11489345 |
| zardaverine | 14.92 | 0.491 | 1.567 | −0.675 | −0.382 | 1.406 | 0.166 | 0.022 | 0.060 | −0.077 | 2372 | 11468125 |
| meloxicam | 20 | −0.324 | 0.573 | −0.329 | −0.929 | 0.224 | 0.207 | −1.255 | −0.850 | −1.857 | 2407 | 11488757 |
| procainamide | 17 | 0.503 | 1.195 | −0.573 | −1.152 | 0.773 | 0.492 | −0.328 | −0.116 | −0.696 | 2431 | 11467485 |
| procainamide | 20 | −1.102 | 1.117 | −1.144 | −0.366 | 0.351 | 1.015 | −0.044 | 0.057 | −0.247 | 2431 | 11489111 |
| chrysanthemic acid | 20 | −0.009 | 0.472 | −0.908 | 0.060 | 0.472 | 0.611 | −0.693 | −0.657 | −0.598 | 2475 | 11489498 |
| diazinon | 20 | −0.010 | 0.074 | −1.251 | −0.558 | 0.998 | 0.298 | −1.047 | −0.762 | −1.349 | 2476 | 11489042 |
| ethion | 20 | 1.318 | 0.899 | −1.264 | −0.987 | 0.841 | 0.432 | 0.404 | 0.487 | 0.229 | 2477 | 11489041 |
| methyl parathione | 20 | 1.967 | 0.145 | −0.341 | −0.353 | 0.218 | 0.414 | 0.500 | 0.500 | 0.450 | 2478 | 11489665 |
| coumophos | 20 | 3.041 | −0.106 | −0.752 | 0.860 | −0.085 | 0.083 | 0.029 | −0.186 | 0.479 | 2479 | 11489661 |
| azinphos methyl | 20 | 1.987 | 0.997 | −0.974 | 0.280 | 0.702 | 0.900 | 0.452 | 0.405 | 0.482 | 2480 | 11489662 |
| disulfoton | 20 | 0.122 | 0.226 | −1.501 | −0.989 | 0.929 | 0.572 | 0.002 | 0.022 | −0.005 | 2481 | 11489672 |
| mevinphos | 20 | 0.130 | 0.496 | −1.230 | −0.859 | 1.071 | 0.643 | −0.949 | −1.016 | −0.592 | 2482 | 11489673 |
| naled | 20 | −0.669 | −0.307 | −1.559 | −0.313 | 0.120 | 0.966 | 0.273 | 0.432 | −0.065 | 2483 | 11489674 |
| dichlorvos | 20 | −0.805 | 0.791 | −2.000 | −0.589 | 0.668 | 0.165 | 0.118 | 0.375 | −0.339 | 2484 | 11489043 |
| oxdemetonmethyl | 20 | −0.511 | 0.724 | −0.494 | −0.255 | 0.833 | 0.196 | 0.122 | 0.246 | −0.125 | 2485 | 11489675 |
| dimethoate | 20 | 0.942 | 0.487 | −1.273 | 0.139 | 1.036 | 0.837 | −1.431 | −1.309 | −1.363 | 2486 | 11489677 |
| malathion | 20 | 0.419 | 1.406 | −1.414 | 0.397 | 1.064 | −0.938 | −0.209 | −0.397 | 0.287 | 2487 | 11489044 |
| phosalone | 20 | 2.358 | 0.897 | −0.750 | −0.407 | −1.945 | 0.522 | −0.767 | −0.578 | −0.959 | 2488 | 11489678 |
| methamidophos | 20 | 2.972 | 1.038 | −0.710 | −0.473 | 0.289 | 0.635 | −1.162 | −0.894 | −1.435 | 2489 | 11489679 |
| phorate | 20 | 0.649 | 0.220 | −0.526 | −0.116 | 0.740 | −0.612 | 0.282 | 0.133 | 0.562 | 2490 | 11489676 |
| dacthal | 20 | −0.954 | −0.561 | −2.116 | −0.046 | 0.253 | −0.159 | −0.916 | −1.062 | −0.402 | 2491 | 11489692 |
| propazine | 20 | −0.634 | −0.606 | −1.928 | −0.648 | 0.725 | −0.083 | −0.837 | −0.761 | −0.783 | 2492 | 11489693 |
| propanil | 20 | −0.873 | −0.530 | −1.657 | −0.091 | −0.085 | −0.108 | 0.576 | 0.574 | 0.511 | 2493 | 11489694 |
| simazine | 20 | −0.698 | −0.125 | −0.829 | −0.390 | 0.382 | −0.360 | −0.386 | −0.400 | −0.238 | 2494 | 11489695 |
| atrazine | 20 | −0.129 | 0.081 | −0.347 | −0.236 | 0.634 | −0.617 | −0.342 | −0.242 | −0.436 | 2495 | 11489696 |
| diuron | 20 | −0.314 | 0.513 | −1.203 | −0.105 | 1.122 | 0.530 | 0.400 | 0.580 | 0.070 | 2496 | 11489045 |
| tebuthiuron | 20 | −0.844 | 0.546 | −0.407 | −0.317 | 1.164 | 0.051 | −1.404 | −1.187 | −1.523 | 2497 | 11489697 |
| dicamba | 20 | −0.500 | −0.078 | −1.617 | −0.328 | 0.818 | 0.278 | −1.740 | −1.356 | −2.129 | 2498 | 11489698 |
| benfluralin | 20 | −1.241 | 0.210 | −1.927 | −0.527 | 0.989 | −0.193 | −0.791 | −0.809 | −0.558 | 2499 | 11489699 |
| prometon | 20 | −0.783 | −0.867 | −2.162 | −0.905 | 1.142 | −0.259 | −0.744 | −0.632 | −0.790 | 2500 | 11489700 |
| metolachlor | 20 | −1.168 | −0.705 | −2.217 | −0.659 | 0.959 | 0.156 | −0.487 | −0.279 | −0.763 | 2501 | 11489701 |
| dichlobenil | 20 | −0.587 | −0.660 | −1.515 | −0.543 | −0.032 | −0.025 | −0.784 | −0.670 | −0.819 | 2502 | 11489702 |
| prometryn | 20 | −0.336 | −0.095 | −1.458 | −0.891 | 1.172 | 0.093 | −0.288 | −0.130 | −0.514 | 2503 | 11489703 |
| trifluralin | 20 | −0.731 | −0.083 | −1.149 | −0.333 | 0.868 | −0.378 | −0.480 | −0.577 | −0.147 | 2504 | 11489704 |
| bentazon | 20 | −0.857 | −0.173 | −0.852 | −0.531 | 0.539 | −0.324 | −1.017 | −0.977 | −0.850 | 2505 | 11489705 |
| 2,4-dichlorophenoxyacetic acid | 20 | −0.123 | 0.028 | −1.450 | −0.625 | 0.554 | 0.309 | −0.423 | −0.237 | −0.669 | 2506 | 11489671 |
| 2,4-dichlorophenoxybutyric acid | 20 | 0.337 | −0.337 | −1.368 | −0.479 | 0.758 | 0.711 | −0.476 | −0.439 | −0.423 | 2507 | 11489670 |
| 2,4,5-trichlorophenoxyacetic acid | 20 | −0.192 | 0.831 | −1.619 | −0.215 | 1.217 | 0.531 | −0.669 | −0.420 | −0.977 | 2508 | 11489040 |
| alachlor | 20 | −3.839 | −7.372 | −3.820 | −1.048 | −0.917 | −5.053 | −2.717 | −2.562 | −2.460 | 2509 | 11489669 |
| 2,4-dichlorophenoxyacetic acid, methyl ester | 20 | 0.777 | 0.234 | −0.975 | −0.464 | 0.503 | 0.708 | −1.200 | −0.982 | −1.362 | 2510 | 11489667 |
| 2,4-dichlorophenoxybutyric acid, methyl ester | 20 | 0.178 | −0.331 | −0.845 | −0.247 | 0.476 | 0.749 | −0.401 | −0.336 | −0.426 | 2511 | 11489668 |
| 2,4,5-trichlorophenoxyacetic acid, methyl ester | 20 | 0.801 | 0.136 | −0.691 | 0.201 | −0.070 | −0.066 | 1.089 | 1.061 | 0.964 | 2512 | 11489666 |
| glyphosate | 20 | 0.663 | 1.910 | −0.806 | −0.537 | 0.189 | 1.051 | 0.944 | 1.048 | 0.584 | 2513 | 11489663 |
| 2,4-dichlorophenoxyacetic acid, isooctyl ester | 20 | 0.656 | 0.224 | −0.700 | −0.029 | −0.120 | 0.575 | −0.116 | 0.002 | −0.296 | 2514 | 11489664 |
| 2,4,5-trichlorophenoxyacetic acid, isooctyl ester | 20 | 1.258 | 0.541 | −0.607 | 0.066 | 0.118 | 0.997 | −0.759 | −0.689 | −0.713 | 2515 | 11489657 |
| chlorpropham | 20 | 0.050 | 0.553 | −1.623 | −0.832 | 0.158 | 0.110 | 0.316 | 0.598 | −0.384 | 2516 | 11487860 |
| propachlor | 20 | −5.583 | −8.358 | −6.698 | −4.270 | −3.747 | −4.882 | −3.430 | −4.130 | −1.250 | 2517 | 11489658 |
| S,S,S,-tributylphosphorotrithioate | 20 | 4.229 | 1.660 | −0.580 | 0.275 | −0.757 | 0.365 | −1.911 | −1.877 | −1.570 | 2518 | 11489659 |
| triallate | 20 | 0.579 | 0.532 | −1.481 | −0.214 | 0.153 | 0.374 | −0.246 | −0.231 | −0.192 | 2519 | 11489660 |
| paradichlorobenzene | 20 | −0.777 | 0.640 | −1.083 | −0.184 | 1.100 | 0.599 | 0.050 | 0.050 | 0.030 | 2520 | 11489685 |
| pentachlorophenol | 20 | −2.253 | −5.275 | 2.658 | −3.529 | 0.192 | −3.176 | −0.930 | −0.905 | −0.763 | 2521 | 11489686 |
| carbofuran | 20 | 2.257 | 0.452 | −1.613 | −0.481 | 0.504 | 0.315 | −0.925 | −0.770 | −1.020 | 2522 | 11489687 |
| chlorpyrifos | 20 | 0.980 | 0.799 | −1.253 | 0.032 | 0.455 | −0.067 | −0.534 | −0.446 | −0.545 | 2523 | 11489046 |
| acephate | 20 | −0.491 | −0.395 | −1.983 | −0.831 | 0.882 | 0.136 | 0.002 | −0.018 | 0.085 | 2524 | 11489541 |
| temefos | 20 | −0.761 | −0.378 | −1.645 | −0.119 | 0.681 | 0.316 | −1.403 | −1.230 | −1.431 | 2527 | 11489689 |
| bendiocarb | 20 | 0.816 | −0.186 | −1.742 | −0.553 | 1.492 | −0.178 | −0.141 | 0.016 | −0.390 | 2528 | 11489542 |
| fenthion | 20 | 0.594 | 0.527 | −1.111 | 0.015 | 1.063 | 0.845 | −0.270 | −0.234 | −0.224 | 2529 | 11489047 |
| ethoprop | 20 | 0.070 | −0.054 | −2.177 | −0.500 | 0.988 | 0.271 | −0.222 | 0.003 | −0.595 | 2530 | 11489690 |
| propoxur | 20 | 2.120 | 0.743 | −1.580 | −0.736 | 0.679 | −0.200 | −0.473 | −0.266 | −0.729 | 2531 | 11489048 |
| propargite | 20 | −0.582 | 0.065 | −2.401 | −0.388 | 0.208 | 0.130 | −0.985 | −1.134 | −0.455 | 2532 | 11489691 |
| dichlorodiphenyltrichloroethane | 20 | −1.165 | 0.206 | −1.627 | −0.807 | 0.757 | 0.399 | −0.430 | −0.013 | −1.105 | 2533 | 11489049 |
| dichlorodiphenyldichloroethylene | 20 | −0.282 | −0.072 | −1.949 | −0.753 | 3.563 | 0.831 | −0.433 | −0.302 | −0.572 | 2534 | 11489681 |
| toxaphene | 20 | −1.096 | −1.562 | −2.310 | 0.375 | −0.885 | −0.903 | −0.017 | 0.239 | −0.491 | 2535 | 11489683 |
| chlordane | 20 | −0.052 | 0.691 | −0.712 | 0.713 | 1.698 | −0.640 | −0.632 | −0.565 | −0.598 | 2536 | 11489684 |
| methoxychlor | 20 | −0.656 | −0.734 | −0.510 | −0.180 | 0.503 | −0.578 | −1.000 | −0.934 | −0.885 | 2537 | 11489706 |
| heptachlor | 20 | −0.375 | 0.049 | 0.387 | 0.337 | 0.601 | 0.657 | −0.593 | −0.449 | −0.729 | 2538 | 11489707 |
| strobane | 20 | −0.034 | −0.266 | −1.114 | −0.727 | 0.352 | −0.198 | −0.368 | −0.234 | −0.535 | 2539 | 11489708 |
| aldrin | 20 | −0.489 | 0.264 | −1.145 | −0.757 | 0.701 | 0.343 | −1.254 | −1.009 | −1.458 | 2540 | 11489710 |
| endosulfan | 20 | −1.047 | 0.622 | −1.178 | 0.278 | 0.558 | 0.482 | −0.283 | −0.161 | −0.434 | 2541 | 11489709 |
| benzylbutylphthalate | 20 | −1.041 | −0.007 | −1.437 | −0.679 | −0.168 | −0.174 | −0.454 | −0.411 | −0.407 | 2542 | 11489621 |
| 4-nonylphenol | 20 | 0.969 | 1.011 | 0.389 | 0.271 | −0.420 | 0.444 | 0.176 | 0.683 | −0.843 | 2543 | 11489648 |
| acetochlor | 20 | −0.632 | 0.978 | 0.226 | 0.015 | −0.124 | 0.075 | −0.260 | −0.318 | −0.045 | 2544 | 11489731 |
| dimethyl 4,4-o-phenylene-bis | 20 | −0.641 | −1.181 | −1.142 | 0.613 | 0.454 | 0.393 | −0.632 | −0.380 | −1.036 | 2546 | 11488462 |
| sanguinarine | 12.04 | −5.346 | −8.386 | −5.277 | −3.957 | −1.134 | −2.136 | −1.550 | −2.600 | 0.830 | 2549 | 11468135 |
| sanguinarine | 20 | −1.023 | −1.435 | −2.258 | −0.858 | −0.697 | −5.748 | −2.099 | 0.009 | −6.006 | 2549 | 11488540 |
| chloramphenicol | 20 | −0.216 | 1.169 | −0.228 | −0.337 | −0.659 | 0.489 | 0.333 | 0.087 | 0.718 | 2550 | 11487899 |
| primaquine | 15.42 | −5.256 | −8.264 | −6.088 | −3.330 | −3.563 | 1.155 | 0.595 | 0.622 | 0.415 | 2551 | 11467624 |
| primaquine | 20 | 0.984 | 2.199 | −1.333 | 0.152 | 0.792 | −5.704 | 0.743 | 0.674 | 0.708 | 2551 | 11488703 |
| 1,2-dimethylhydrazine | 20 | −1.633 | 0.180 | −1.531 | −0.907 | 1.083 | 0.303 | 0.739 | 0.717 | 0.626 | 2553 | 11488593 |
| conessine | 20 | −0.739 | 0.461 | −1.459 | −0.262 | 0.970 | 1.197 | −1.055 | −1.058 | −0.871 | 2554 | 11488731 |
| diaziquone | 20 | −0.617 | 0.938 | −1.450 | −0.438 | 0.106 | −0.829 | 0.398 | 0.365 | 0.469 | 2555 | 11489002 |
| methylmethane | 20 | −1.159 | 0.234 | −1.330 | −0.924 | 0.909 | 0.221 | −0.516 | −0.362 | −0.754 | 2557 | 11488733 |
| benzo[a]pyrene | 20 | 0.501 | 0.381 | −0.946 | −0.254 | 1.095 | 0.652 | 0.246 | 0.366 | 0.020 | 2558 | 11488897 |
| cadmium acetate | 20 | −1.393 | 0.954 | −1.896 | −1.276 | −1.066 | −2.166 | 1.464 | 1.340 | 1.491 | 2559 | 11488294 |
| 3-methylcholanthrene | 20 | −1.203 | 0.397 | −1.878 | −0.411 | 0.849 | 0.846 | −0.168 | 0.112 | −0.629 | 2560 | 11488910 |
| 2,4-dinitrophenol | 20 | −1.024 | −1.036 | −0.839 | 0.489 | 1.402 | 0.031 | −0.088 | −0.131 | −0.003 | 2561 | 11488489 |
| penicillic acid | 20 | −1.082 | 0.598 | −0.634 | −0.210 | −1.003 | −0.243 | −1.311 | −1.206 | −1.244 | 2565 | 11488407 |
| desmethyldihydrocapsaicin | 20 | 0.046 | 0.130 | −1.248 | −0.672 | 0.636 | 0.605 | −0.576 | −0.497 | −0.563 | 2566 | 11488907 |
| dichlorphenamide | 13.1 | 0.692 | 0.479 | −1.076 | −0.465 | 0.222 | 0.842 | −0.442 | −0.401 | −0.441 | 2570 | 11467957 |
| tubocurarine | 20 | −0.589 | 0.899 | −0.749 | −0.866 | 0.451 | 0.387 | −0.325 | −0.272 | −0.370 | 2572 | 11489151 |
| tinidazole | 16.18 | −1.120 | 0.167 | −1.767 | −0.967 | 0.480 | 0.150 | −0.611 | −0.104 | −1.508 | 2575 | 11467914 |
| tinidazole | 20 | −1.121 | 1.810 | −0.734 | 0.347 | 0.558 | −0.492 | 0.054 | 0.148 | −0.174 | 2575 | 11488464 |
| benzyl isothiocyanate | 20 | −0.764 | −0.015 | −1.178 | −0.141 | −0.803 | −0.443 | −0.932 | −0.755 | −1.123 | 2576 | 11488668 |
| thiodiglycol | 20 | −0.423 | 3.392 | −1.250 | 0.184 | 0.861 | 1.028 | −0.492 | −0.456 | −0.426 | 2579 | 11488223 |
| ticarcillin | 10.4 | 0.216 | 1.089 | −0.127 | 0.047 | 0.415 | 0.002 | 0.183 | 0.315 | −0.126 | 2586 | 11468215 |
| crotamiton | 19.68 | 1.013 | 1.174 | 1.423 | −1.055 | 0.190 | 0.084 | 0.653 | 0.997 | −0.184 | 2660 | 11468099 |
| crotamiton | 20 | −0.270 | 0.010 | −0.870 | −1.322 | 0.638 | 0.073 | −0.044 | 0.319 | −0.742 | 2660 | 11489516 |
| iodipamide | 3.5 | −0.961 | −0.669 | −0.253 | 0.039 | 0.913 | −0.891 | 0.060 | −0.070 | 0.290 | 2685 | 11468087 |
| epirizole | 17.08 | −0.756 | 0.455 | −1.614 | −0.525 | 0.765 | −0.453 | −1.362 | −1.296 | −1.279 | 2702 | 11467180 |
| pyridoxine | 23.64 | −0.182 | −0.160 | −1.535 | −0.421 | 0.937 | 0.286 | 0.246 | 0.149 | 0.398 | 2709 | 11467771 |
| ethynylestradiol 3-methyl ether | 12.88 | −0.071 | −0.281 | −0.915 | −0.824 | 0.867 | 0.724 | −0.938 | −0.690 | −1.272 | 2710 | 11467994 |
| testosterone propionate | 11.62 | 0.371 | 1.906 | −0.649 | −0.434 | −0.576 | 0.346 | −0.784 | −0.579 | −1.063 | 2717 | 11467549 |
| hymecromone | 22.7 | 0.273 | 0.379 | 0.139 | −0.454 | 0.284 | 1.733 | 1.085 | 0.979 | 1.081 | 2732 | 11468049 |
| ozagrel | 17.52 | 0.168 | 0.739 | −0.543 | −0.086 | 1.631 | 0.251 | 0.882 | 0.807 | 0.867 | 2742 | 11468127 |
| metyrapone | 17.68 | −1.204 | −0.176 | −0.539 | −0.268 | −0.087 | 0.372 | 0.354 | 0.375 | 0.234 | 2743 | 11468052 |
| zalcitabine | 18.94 | −0.670 | 0.005 | −0.535 | −0.518 | 0.899 | 0.157 | 0.059 | 0.048 | 0.066 | 2747 | 11468185 |
| methotrimeprazine | 12.18 | −0.002 | −0.906 | −1.154 | −0.140 | 0.033 | −0.199 | 0.223 | 0.177 | 0.274 | 2752 | 11467945 |
| etidronic acid | 19.42 | −0.135 | −0.143 | −1.408 | −0.600 | 1.192 | 0.462 | 0.026 | 0.022 | 0.025 | 2764 | 11468011 |
| felbinac | 18.84 | −0.265 | 1.401 | −0.648 | 0.052 | 2.434 | 0.714 | −0.928 | −0.838 | −0.949 | 2776 | 11468041 |
| clebopride | 10.7 | −0.607 | −0.674 | −0.911 | 0.709 | 1.496 | 0.179 | 0.070 | −0.044 | 0.284 | 2777 | 11467528 |
| clebopride | 20 | −0.658 | 0.232 | −1.789 | −0.237 | 1.468 | 0.602 | 0.323 | 0.406 | 0.078 | 2777 | 11488583 |
| canrenoic acid | 11.16 | 0.484 | −0.790 | −0.668 | −0.667 | 1.092 | −0.361 | 0.474 | 0.407 | 0.485 | 2784 | 11467296 |
| indomethacin | 11.18 | −0.249 | −0.314 | −1.259 | −0.535 | 2.161 | −2.360 | −0.207 | −0.199 | −0.172 | 2797 | 11467420 |
| indomethacin | 20 | 1.222 | 0.596 | −1.394 | 1.943 | 0.846 | 0.107 | 1.771 | 1.318 | 2.396 | 2797 | 11488786 |
| carmofur | 20 | −0.126 | 0.283 | −2.715 | −0.528 | −0.305 | −0.087 | −0.338 | −0.352 | −0.283 | 2801 | 11487842 |
| bemegride | 25.78 | −0.962 | 2.019 | 0.024 | 0.808 | 0.108 | 0.970 | −0.279 | −0.069 | −0.658 | 2819 | 11468030 |
| domperidone | 9.4 | 0.888 | 0.194 | −0.331 | 0.126 | 0.152 | 0.276 | −0.099 | −0.029 | −0.234 | 2830 | 11467609 |
| S(+)-terguride | 11.74 | −0.531 | −0.681 | −0.964 | −0.585 | 0.083 | −0.138 | 0.926 | 0.474 | 1.666 | 2844 | 11468093 |
| moxisylyte | 14.32 | 0.426 | −0.135 | −1.330 | −0.807 | 0.441 | −0.330 | −0.147 | −0.127 | −0.207 | 2847 | 11467190 |
| cilostazol | 20 | −0.200 | 0.623 | −0.215 | −0.908 | 2.587 | 0.481 | 0.157 | −0.113 | 0.736 | 2857 | 11488934 |
| benzbromarone | 9.44 | −0.067 | 0.079 | 0.360 | −1.098 | 0.591 | 0.340 | 0.195 | 0.088 | 0.380 | 2873 | 11467518 |
| glutamine | 20 | −0.030 | 0.880 | −0.888 | −0.851 | 2.225 | 0.452 | 0.213 | 0.208 | 0.182 | 2880 | 11489193 |
| cyclacillin | 11.72 | −0.221 | −0.257 | 1.769 | −0.107 | −0.360 | −0.886 | 0.039 | −0.069 | 0.250 | 2884 | 11468268 |
| meticrane | 14.52 | 0.323 | 1.068 | −1.627 | −0.012 | 0.443 | 0.527 | 0.003 | 0.066 | −0.179 | 2898 | 11467159 |
| trimethadione | 27.94 | −1.300 | 0.162 | −1.825 | −0.503 | 1.082 | 0.085 | −0.930 | −0.741 | −1.139 | 2900 | 11467663 |
| dosulepin | 13.54 | 1.339 | 0.875 | −0.806 | 0.069 | 0.095 | 0.997 | −0.215 | 0.065 | −0.761 | 2911 | 11467636 |
| trapidil | 19.48 | −0.011 | 0.155 | −0.788 | −0.215 | 0.898 | 0.082 | −0.158 | −0.039 | −0.378 | 2920 | 11468160 |
| bromperidol | 9.52 | 1.393 | 0.434 | −0.005 | 0.033 | 1.607 | 0.998 | 0.081 | 0.060 | 0.099 | 2922 | 11467657 |
| iodipamide | 20 | 0.451 | −0.423 | −1.303 | −0.147 | 1.515 | −0.253 | 0.729 | 0.633 | 0.860 | 2935 | 11488886 |
| ioxaglic acid | 3.16 | −0.293 | 1.071 | −0.820 | −0.321 | 0.042 | 0.422 | −0.333 | −0.355 | −0.237 | 2957 | 11468210 |
| dilazep | 6.62 | −1.111 | −0.641 | −1.284 | −0.865 | 0.972 | −0.150 | 0.109 | −0.054 | 0.371 | 2997 | 11467384 |
| diphenidol | 12.92 | −0.622 | 1.158 | −0.364 | −0.471 | 0.308 | 1.413 | 1.217 | 1.221 | 0.975 | 3036 | 11467400 |
| diflorasone diacetate | 8.08 | −0.860 | −0.652 | −1.681 | −0.039 | −0.021 | −0.871 | −0.080 | −0.240 | 0.240 | 3043 | 11467767 |
| alpha-santonin | 16.24 | 0.154 | 0.732 | 0.149 | 0.619 | −0.403 | −1.010 | 0.249 | 0.056 | 0.582 | 3047 | 11468218 |
| santonin | 20 | −1.099 | 0.556 | −1.064 | −0.433 | 0.461 | −0.759 | −0.385 | −0.478 | −0.147 | 3047 | 11488515 |
| guanethidine | 20.18 | 0.543 | 1.295 | −0.740 | −0.504 | 0.048 | 0.290 | 0.135 | 0.031 | 0.319 | 3055 | 11467465 |
| guanethidine | 20 | −0.898 | 0.054 | −1.416 | −0.594 | 0.976 | −0.026 | 0.145 | 0.224 | 0.027 | 3055 | 11488919 |
| panthenol (D) | 19.48 | −0.109 | 0.699 | −1.407 | −0.466 | 0.430 | 0.048 | −1.611 | −1.685 | −1.206 | 3060 | 11467170 |
| cefoperazone | 6.2 | −0.399 | 2.066 | −0.776 | −0.184 | 0.148 | 0.967 | 0.346 | 0.437 | 0.075 | 3063 | 11467475 |
| methimazole | 35.04 | 0.022 | −0.033 | −1.626 | −0.369 | 0.106 | −0.372 | −0.090 | −0.159 | 0.065 | 3092 | 11467934 |
| hydrocotarnine | 18.08 | −0.296 | −0.016 | −2.202 | −0.818 | 0.946 | −0.062 | 0.177 | 0.287 | −0.092 | 3100 | 11467753 |
| hydrocotarnine | 20 | −1.349 | −0.397 | −1.162 | −0.841 | 0.878 | 0.124 | 0.560 | 0.356 | 0.861 | 3100 | 11489209 |
| flavoxate | 10.22 | −1.126 | 2.109 | −0.174 | −0.324 | 0.521 | 1.276 | −0.112 | 0.059 | −0.431 | 3101 | 11467390 |
| benoxinate | 12.96 | −0.753 | −0.177 | −0.975 | −0.410 | 1.135 | −0.489 | 0.348 | 0.588 | −0.254 | 3127 | 11467205 |
| dydrogesterone | 12.8 | 0.425 | −0.222 | −0.997 | −0.554 | 0.729 | 0.873 | −0.141 | −0.090 | −0.220 | 3129 | 11467819 |
| rescinnamin | 6.3 | 1.773 | 2.812 | −0.246 | −0.048 | 1.685 | 0.566 | 0.378 | 0.262 | 0.541 | 3141 | 11467716 |
| piretanide | 11.04 | 0.737 | 2.322 | −0.288 | −0.577 | −0.655 | 0.188 | −0.246 | −0.212 | −0.290 | 3168 | 11468195 |
| lisuride | 11.82 | −0.329 | 1.157 | −2.069 | −0.838 | 0.023 | 0.617 | −0.899 | −0.922 | −0.723 | 3169 | 11467254 |
| cinnarazine | 10.86 | −0.692 | 1.178 | −0.009 | −0.711 | 1.252 | −0.519 | 0.362 | 0.338 | 0.349 | 3172 | 11467426 |
| prothionamide | 20 | 0.236 | 1.276 | −1.091 | −0.056 | 1.300 | 0.156 | −0.310 | −0.318 | −0.282 | 3182 | 11487835 |
| acetohexamide | 12.34 | −1.368 | −0.025 | −1.503 | −1.107 | 1.642 | −0.453 | −0.862 | −0.945 | −0.568 | 3186 | 11467203 |
| procarbazine | 18.08 | 1.082 | 0.133 | 0.595 | 0.240 | −0.880 | −0.008 | 1.248 | 1.288 | 0.922 | 3199 | 11468260 |
| urapidil | 10.32 | −0.651 | −0.283 | −0.597 | −0.538 | 0.411 | 0.487 | −0.066 | −0.021 | −0.161 | 3202 | 11468053 |
| urapidil | 20 | −0.316 | −0.374 | −0.668 | −0.361 | 0.438 | 0.151 | −0.168 | 0.035 | −0.486 | 3202 | 11488988 |
| salsalate | 20 | −0.591 | 0.602 | −1.448 | −0.733 | 0.320 | 0.435 | −1.090 | −0.859 | −1.360 | 3235 | 11488509 |
| batyl alcohol | 20 | −0.197 | 1.347 | 0.138 | 0.201 | −0.324 | 0.278 | −0.371 | −0.686 | 0.366 | 3250 | 11489425 |
| alverine citrate | 14.22 | 1.004 | 0.883 | 0.006 | −0.087 | 1.231 | 1.451 | −0.641 | −0.545 | −0.756 | 3256 | 11467322 |
| mephentermine | 24.5 | 0.521 | 0.503 | −0.784 | −0.591 | −0.319 | 0.554 | −0.097 | 0.018 | −0.319 | 3263 | 11467874 |
| mephentermine | 20 | −0.969 | 0.911 | −0.450 | −1.120 | 0.882 | 1.371 | −0.210 | −0.021 | −0.513 | 3263 | 11488290 |
| cefamandole | 20 | −0.861 | 0.335 | −0.518 | −0.177 | −0.328 | 0.439 | 0.018 | 0.237 | −0.428 | 3264 | 11489279 |
| phenelzine | 29.38 | 0.907 | 0.744 | −0.341 | 0.015 | 0.544 | −0.707 | 0.527 | 0.552 | 0.325 | 3273 | 11467318 |
| phenelzine | 20 | 0.149 | 0.907 | −0.744 | −0.485 | 0.608 | 0.437 | −0.938 | −0.955 | −0.629 | 3273 | 11488825 |
| ketanserin | 20 | −1.051 | −0.727 | −0.987 | −0.439 | 1.244 | 0.509 | −1.015 | −0.935 | −0.952 | 3304 | 11489529 |
| cyproheptadine | 13.92 | −0.558 | 0.745 | −1.321 | 0.430 | 0.366 | 0.064 | −1.166 | −0.919 | −1.483 | 3326 | 11467251 |
| guanfacine | 16.26 | −0.015 | 1.119 | −1.173 | −0.622 | 1.249 | 0.613 | 0.496 | 0.638 | 0.110 | 3368 | 11467487 |
| thiamine | 15.08 | −0.247 | 0.706 | 0.145 | −0.160 | 1.222 | 0.284 | 0.259 | 0.161 | 0.412 | 3382 | 11467779 |
| isocarboxazid | 17.3 | −0.205 | −0.386 | −0.860 | −0.637 | 0.106 | −0.005 | −0.325 | −0.340 | −0.230 | 3383 | 11467943 |
| (−)-levobunolol | 13.72 | −0.923 | 0.019 | −1.716 | −0.772 | 1.403 | 0.049 | −0.595 | −0.647 | −0.372 | 3452 | 11467995 |
| umbelliferone | 20 | 0.137 | 0.726 | −1.479 | −1.303 | 0.545 | −0.272 | −0.788 | −0.661 | −0.935 | 3526 | 11489778 |
| guvacine | 20 | −1.458 | −0.056 | −1.711 | −0.677 | −0.161 | 0.280 | −0.412 | −0.678 | 0.201 | 3684 | 11489290 |
| dimaprit | 24.8 | 0.169 | 0.490 | −0.416 | 2.136 | 0.016 | −0.184 | −0.311 | −0.403 | −0.076 | 3723 | 11468131 |
| decamethonium bromide | 15.48 | 0.213 | 1.342 | 1.618 | 0.049 | 1.665 | 0.967 | 0.938 | 0.940 | 0.744 | 3855 | 11468116 |
| mecamylamine | 23.92 | 1.457 | −0.130 | 0.560 | −0.349 | −0.583 | −0.062 | 0.123 | 0.389 | −0.447 | 3856 | 11468259 |
| ciprofibrate | 13.84 | −0.930 | 0.365 | −0.632 | −0.154 | 0.271 | −0.078 | 0.572 | 0.421 | 0.763 | 3903 | 11468224 |
| carprofen | 20 | −0.827 | 0.752 | −1.838 | −0.481 | 0.724 | 0.138 | −0.857 | −0.801 | −0.729 | 4164 | 11489052 |
| isoetharine | 16.72 | −0.131 | 0.237 | −0.882 | −0.965 | −0.727 | −0.262 | −0.412 | −0.399 | −0.363 | 4338 | 11467897 |
| loxapine | 12.2 | 1.467 | 0.168 | −1.459 | −0.312 | −0.254 | 0.366 | −0.849 | −0.766 | −0.897 | 4362 | 11467280 |
| loxapine | 20 | 1.067 | −0.106 | −0.898 | −0.731 | 1.305 | −0.072 | −0.311 | −0.387 | −0.065 | 4362 | 11489553 |
| megestrol acetate | 10.4 | 0.304 | 0.577 | 0.085 | 0.141 | 0.953 | −0.308 | 1.288 | 1.214 | 1.175 | 4369 | 11468104 |
| meglumine | 20.5 | −1.025 | 1.957 | −0.525 | 0.293 | 0.274 | 0.969 | −0.117 | −0.089 | −0.143 | 4370 | 11468032 |
| mesoridazine | 10.34 | −0.524 | −0.524 | −0.938 | −0.393 | 0.760 | −0.121 | −0.812 | −0.847 | −0.583 | 4379 | 11467677 |
| methantheline | 11.74 | −0.041 | 1.257 | −0.454 | 0.057 | 0.199 | 0.326 | −0.068 | −0.003 | −0.199 | 4382 | 11468214 |
| oxamniquine | 14.32 | −1.195 | −1.036 | −0.401 | −0.392 | 0.581 | −0.104 | 0.181 | −0.060 | 0.630 | 4425 | 11468174 |
| proguanil | 15.76 | −0.921 | −1.029 | −0.493 | 0.189 | −0.929 | −0.064 | −0.480 | −0.557 | −0.238 | 4480 | 11468147 |
| chlorguanide | 20 | 0.105 | 0.500 | −0.742 | −0.718 | 0.276 | −0.543 | −0.250 | −0.129 | −0.379 | 4480 | 11488951 |
| proparacaine | 13.58 | 0.375 | −0.446 | 0.278 | 0.031 | 0.730 | −1.020 | 1.027 | 0.933 | 1.013 | 4481 | 11468107 |
| protriptyline | 15.18 | −0.906 | −0.995 | 0.049 | 0.508 | −0.007 | −1.335 | 0.719 | 0.778 | 0.438 | 4487 | 11468078 |
| trigonelline | 20 | −1.304 | 0.777 | −1.906 | −0.748 | 0.695 | −0.072 | −0.372 | −0.511 | 0.035 | 4895 | 11488412 |
| fluspirilen | 8.42 | 0.640 | −0.546 | −0.239 | 0.538 | −0.191 | −0.063 | −0.910 | −0.789 | −1.002 | 23081 | 11468054 |
| mexamine | 20 | −0.163 | 0.061 | −0.245 | −0.712 | 1.030 | 0.234 | 0.070 | 0.177 | −0.090 | 52159 | 11488927 |
| 5,7-dichlorokynurenic acid | 20 | 0.384 | 0.128 | −0.711 | −0.406 | 1.628 | −0.534 | 1.079 | 0.900 | 1.186 | 89599 | 11489815 |
| harmine | 18.84 | −0.515 | −0.633 | −2.665 | −0.421 | −0.521 | 0.187 | 0.068 | −0.081 | 0.368 | 297849 | 11467761 |
| harmine | 20 | 0.658 | −0.104 | −2.629 | 0.463 | −0.395 | 0.396 | 0.208 | −0.039 | 0.717 | 297849 | 11488384 |
| 5-fluoroindole-2-carboxylic acid | 20 | −0.059 | −0.383 | −0.968 | −0.373 | 0.371 | −0.043 | −0.105 | −0.284 | 0.279 | 348755 | 11489285 |
| 1-(2-methoxyphenyl)piperazine | 20 | −0.289 | 0.326 | −1.047 | −0.272 | −0.114 | −0.744 | −0.878 | −0.743 | −0.933 | 352677 | 11489634 |
| clemizole | 12.28 | 0.147 | −0.736 | 0.264 | −0.615 | 1.477 | 0.226 | 0.561 | 0.593 | 0.349 | 386963 | 11467375 |
| amodiaquin | 11.24 | 0.263 | −1.051 | −1.100 | −0.547 | 0.192 | −0.247 | 0.506 | 0.360 | 0.720 | 467359 | 11467457 |
| ferulic acid | 20 | −1.181 | 0.133 | −1.254 | −0.844 | −0.272 | 0.169 | −0.380 | −0.432 | −0.191 | 802058 | 11489210 |
| glycocholic acid | 8.6 | −0.392 | 0.088 | −0.789 | −0.577 | 0.470 | 0.143 | −0.950 | −0.778 | −1.118 | 821975 | 11467669 |
| isoliquiritigenin | 20 | 0.088 | 0.601 | −0.121 | 0.011 | 0.228 | 0.089 | −0.987 | −0.856 | −1.073 | 831758 | 11488691 |
| succinylacetone | 20 | −0.870 | −0.284 | −1.695 | −0.770 | 0.905 | 0.901 | −0.771 | −0.884 | −0.337 | 832189 | 11488283 |
| aspartame | 20 | −0.475 | 0.544 | −1.203 | −0.659 | 0.815 | 0.554 | 0.146 | 0.172 | 0.103 | 832325 | 11489522 |
| agmatine | 20 | −0.180 | 2.210 | −0.522 | 0.134 | 1.006 | 0.402 | −0.547 | −0.388 | −0.729 | 839435 | 11489424 |
| 5-aminopentanoic acid | 20 | −0.492 | −0.549 | −1.044 | −0.917 | 0.645 | 0.309 | 0.282 | 0.136 | 0.534 | 840551 | 11489226 |
| anabasine | 24.66 | −0.010 | −0.872 | −0.191 | −1.113 | 2.431 | 0.716 | 0.141 | 0.200 | −0.013 | 852250 | 11467817 |
| anabasine | 20 | −0.552 | −0.450 | −0.128 | −0.672 | 0.352 | 0.635 | −1.317 | −1.009 | −1.645 | 852250 | 11489608 |
| nialamide | 13.4 | −0.341 | 0.092 | 0.025 | 0.394 | 0.058 | −0.726 | 1.166 | 1.235 | 0.796 | 865102 | 11468247 |
| 7-chlorokynurenic acid | 20 | 0.449 | 0.838 | −0.636 | 0.461 | 1.279 | −0.539 | 0.185 | 0.139 | 0.247 | 873168 | 11489286 |
| 7-chloroethyltheophylline | 20 | −0.272 | −0.243 | −0.681 | −0.631 | −0.025 | −0.182 | −1.393 | −1.263 | −1.337 | 907089 | 11489633 |
| alaproclate | 20 | −0.052 | −0.288 | −0.621 | −0.073 | 0.591 | 0.372 | −0.717 | −0.426 | −1.124 | 907120 | 11489469 |
| N,N-dimethylamiloride | 20 | −0.932 | 0.150 | −1.283 | −0.618 | 0.309 | 0.249 | −0.096 | 0.113 | −0.468 | 907149 | 11489431 |
| N,N-hexamethyleneamiloride | 20 | 0.387 | −1.805 | −1.069 | −0.285 | 0.428 | −0.256 | −0.015 | −0.128 | 0.273 | 907181 | 11489485 |
| 2-(2,6-dimethoxyphenoxyethyl)aminomethyl-1,4-benzodioxane | 20 | −0.819 | −0.657 | −1.987 | −0.474 | 1.695 | −0.140 | 0.010 | −0.031 | 0.100 | 907188 | 11489391 |
| bretylium | 16.46 | −0.520 | −0.045 | −0.192 | −0.347 | 0.191 | 0.295 | 0.113 | 0.245 | −0.195 | 907192 | 11468090 |
| buflomedil | 13.02 | −0.432 | 1.151 | −0.385 | −0.688 | 0.537 | 0.464 | −0.015 | 0.037 | −0.117 | 907205 | 11467574 |
| clofilium | 11.8 | −2.024 | −5.798 | −4.093 | 0.584 | −2.010 | −3.111 | −0.510 | −0.876 | 0.338 | 907228 | 11467467 |
| GBR 12909 | 8.88 | −1.147 | −0.535 | −0.212 | 0.556 | 1.362 | 0.644 | 0.566 | −0.002 | 1.620 | 907273 | 11467534 |
| debrisoquin sulfate | 22.82 | −0.349 | −0.410 | −1.458 | −0.615 | 0.684 | −0.125 | −0.782 | −0.655 | −0.882 | 907283 | 11467520 |
| dihydroergocristine | 6.54 | −0.726 | 1.259 | −0.970 | 1.322 | 0.624 | 1.286 | −0.183 | −0.292 | 0.077 | 907285 | 11467710 |
| (−)-eseroline | 18.32 | 0.123 | −1.664 | −0.982 | 0.158 | −0.538 | −0.565 | 0.087 | 0.144 | −0.056 | 907302 | 11468230 |
| epigallocatechin | 20 | −1.489 | 0.559 | −1.197 | −2.502 | −0.531 | 0.046 | −0.238 | −0.133 | −0.418 | 907310 | 11488519 |
| famprofazone | 10.6 | 1.369 | −0.725 | −1.707 | −0.633 | 0.919 | 0.525 | 0.265 | 0.223 | 0.297 | 907320 | 11467851 |
| hemicholinium | 9.64 | −0.716 | 0.385 | −1.279 | −0.412 | 1.071 | 0.136 | 0.273 | 0.364 | 0.047 | 907335 | 11467541 |
| lidoflazine | 8.14 | 0.280 | −0.611 | −0.424 | −0.165 | 0.968 | −0.101 | −0.703 | −0.647 | −0.679 | 907366 | 11467529 |
| lorglumide | 8.7 | −0.989 | 0.392 | 0.105 | −0.220 | 0.174 | 0.058 | −0.281 | −0.423 | 0.057 | 907370 | 11468063 |
| dizocilpine | 18.08 | 0.028 | −0.120 | −0.772 | −0.328 | 0.684 | −0.006 | −0.530 | −0.557 | −0.409 | 907387 | 11467257 |
| meprylcaine | 17 | −0.674 | 0.647 | −1.775 | −0.319 | 0.180 | 0.336 | −0.500 | −0.470 | −0.480 | 907413 | 11468212 |
| nisoxetine | 14.74 | 0.290 | −0.479 | 0.123 | 0.268 | −0.042 | −0.937 | 0.422 | 0.277 | 0.636 | 907434 | 11468058 |
| pirenperone | 10.16 | 0.385 | 0.486 | −1.310 | −0.314 | 0.470 | −0.623 | −0.417 | −0.337 | −0.502 | 907463 | 11467679 |
| pirenperone | 20 | −0.167 | 1.471 | 0.205 | 0.099 | 0.890 | 0.319 | 0.181 | 0.148 | 0.254 | 907463 | 11489496 |
| (−)-quinpirole | 18.24 | 0.267 | 0.316 | −0.818 | −0.447 | −0.410 | 0.222 | −0.334 | −0.175 | −0.598 | 907479 | 11468241 |
| tracazolate | 13.14 | 1.005 | 1.151 | −1.205 | 0.090 | 1.591 | 0.440 | 0.363 | 0.356 | 0.295 | 907524 | 11468124 |
| telenzepine | 10.8 | −0.672 | −0.155 | −1.150 | 0.033 | −0.046 | 0.023 | 0.093 | 0.232 | −0.206 | 907526 | 11467451 |
| tremorine | 20.8 | 0.408 | 1.262 | −0.389 | −0.283 | 1.072 | 0.670 | 0.500 | 0.700 | −0.040 | 907527 | 11467479 |
| isotretinoin | 13.32 | −0.793 | 0.181 | −1.870 | −0.022 | −0.287 | −0.301 | 1.064 | 0.989 | 1.007 | 1000009 | 11467404 |
| emetine | 20 | −1.392 | −0.323 | −3.535 | 1.463 | −3.162 | −0.191 | −0.052 | 1.934 | −4.145 | 1000036 | 11487888 |
| amiloride | 17.42 | −0.180 | 2.179 | −0.998 | −0.350 | 0.689 | 0.137 | 0.202 | 0.378 | −0.233 | 1000042 | 11467155 |
| amiloride | 20 | −1.067 | −1.069 | −1.763 | −0.880 | 1.523 | 0.965 | −0.626 | −0.618 | −0.570 | 1000042 | 11487934 |
| paclitaxel | 20 | 0.528 | 0.812 | −0.628 | −0.106 | −1.967 | −1.583 | 1.683 | 1.916 | 0.861 | 1000045 | 11488688 |
| bepridil | 10.92 | −0.391 | −0.405 | −1.164 | −0.821 | 1.695 | 0.541 | 0.316 | 0.226 | 0.450 | 1000048 | 11467516 |
| bepridil | 20 | 0.890 | 0.753 | −0.802 | −0.687 | 1.217 | −0.436 | −1.443 | −1.250 | −1.563 | 1000048 | 11488717 |
| gramicidin | 20 | −3.206 | −4.404 | −3.832 | −3.957 | −2.491 | −3.179 | −1.904 | −1.890 | −1.510 | 1000054 | 11488892 |
| verapamil | 8.8 | 0.905 | 0.254 | −0.022 | −0.040 | 0.124 | 0.128 | −0.024 | 0.178 | −0.465 | 1000056 | 11467289 |
| verapamil | 20 | 0.686 | −0.638 | −1.004 | −0.385 | 1.279 | 0.053 | −0.812 | −1.037 | −0.159 | 1000056 | 11489556 |
| yohimbine | 20 | −0.411 | 0.317 | −1.180 | −0.854 | −0.067 | 0.135 | −0.766 | −0.464 | −1.253 | 1000060 | 11488482 |
| amethopterin | 8.8 | −0.619 | 0.459 | −2.015 | −1.117 | 0.785 | −0.343 | −0.232 | −0.194 | −0.264 | 1000064 | 11467521 |
| cepharanthine | 20 | −1.186 | 0.450 | −0.947 | −0.297 | −0.071 | 0.057 | −1.259 | −1.111 | −1.332 | 1000069 | 11488648 |
| chenodiol | 10.18 | −0.163 | 0.190 | −1.448 | −0.839 | 0.124 | 0.090 | 0.162 | 0.416 | −0.387 | 1000071 | 11467433 |
| ifosfamide | 15.32 | −1.270 | 0.352 | −2.121 | −0.547 | 1.175 | 0.159 | −0.995 | −0.833 | −1.134 | 1000080 | 11467981 |
| rolipram | 14.52 | −0.116 | 0.352 | −0.854 | −0.743 | 0.302 | −0.367 | 0.476 | 0.693 | −0.077 | 1000092 | 11468072 |
| rosiglitazone | 20 | −0.908 | −0.023 | −0.990 | −0.352 | 0.382 | −0.121 | −0.066 | 0.023 | −0.165 | 1000093 | 11489057 |
| simvastatin | 9.56 | −0.166 | −2.986 | −3.288 | 0.101 | −1.761 | −2.713 | −0.039 | −0.322 | 0.550 | 1000094 | 11468013 |
| simvastatin | 20 | −1.051 | −3.923 | −2.644 | 0.240 | −1.276 | −1.750 | −0.310 | −0.448 | 0.062 | 1000094 | 11489487 |
| tetramisole | 19.58 | −0.035 | 0.468 | −0.507 | −0.712 | 1.088 | 0.209 | −0.045 | 0.000 | −0.124 | 1000096 | 11467693 |
| protoporphyrin IX | 20 | −1.308 | 0.421 | −2.485 | −1.307 | 0.321 | −0.180 | −0.457 | −0.215 | −0.792 | 1000104 | 11488832 |
| bezafibrate | 11.06 | −0.864 | −0.822 | −0.802 | −0.364 | 1.462 | 0.802 | 0.408 | 0.337 | 0.472 | 1000105 | 11467526 |
| bezafibrate | 20 | −1.167 | 0.337 | −1.393 | −0.762 | 0.861 | −0.066 | −0.500 | −0.137 | −1.168 | 1000105 | 11488738 |
| praziquantel | 12.8 | 0.677 | 0.696 | 0.160 | 0.110 | 1.420 | −0.166 | 0.689 | 0.665 | 0.615 | 1000106 | 11467408 |
| praziquantel | 20 | −0.918 | 0.739 | −0.219 | 0.901 | 0.518 | −0.447 | −0.134 | −0.249 | 0.130 | 1000106 | 11489104 |
| norethindrone acetate | 20 | −0.119 | 0.055 | −1.400 | −0.734 | 0.393 | 0.140 | 0.510 | 0.640 | 0.260 | 1000107 | 11488879 |
| nadide | 20 | −0.381 | 0.549 | −1.173 | −0.397 | 1.019 | 0.126 | 0.127 | 0.140 | 0.152 | 1000108 | 11488872 |
| vidarabine | 20 | −0.817 | 0.064 | −1.227 | −0.923 | 0.451 | −0.469 | −0.768 | −0.785 | −0.581 | 1000109 | 11489152 |
| isoreserpine | 20 | 1.421 | 0.495 | 0.926 | −0.748 | 0.869 | 0.366 | 0.194 | 0.131 | 0.306 | 1000110 | 11489586 |
| biotin | 20 | −0.621 | 0.477 | −0.488 | 0.035 | 0.518 | −1.123 | 0.491 | 0.376 | 0.636 | 1000111 | 11489326 |
| colforsin | 20 | 0.685 | 0.921 | 0.699 | −0.647 | 0.064 | −0.130 | −1.181 | −0.680 | −1.990 | 1000112 | 11488687 |
| chloroquine | 12.5 | 0.169 | −0.540 | −1.509 | −0.791 | 0.887 | −0.119 | −0.191 | −0.173 | −0.184 | 1000114 | 11467696 |
| chloroquine | 20 | −0.225 | −1.253 | −2.252 | −1.109 | 1.504 | −0.096 | 0.087 | −0.013 | 0.203 | 1000114 | 11487943 |
| rauwolscine | 11.28 | 0.696 | 1.591 | −0.519 | −1.075 | 0.835 | −1.218 | 0.129 | 0.398 | −0.433 | 1000115 | 11467725 |
| rauwolscine | 20 | 0.279 | 0.379 | −0.143 | −0.092 | 0.697 | 0.671 | 0.099 | 0.202 | −0.150 | 1000115 | 11488686 |
| warfarin | 20 | −0.573 | −0.390 | −1.607 | −0.869 | 1.144 | 0.267 | 0.117 | −0.040 | 0.411 | 1000116 | 11488751 |
| progesterone | 20 | −0.149 | −0.703 | 0.211 | 0.803 | 2.274 | 0.069 | −0.741 | −0.876 | −0.330 | 1000117 | 11489115 |
| pseudoephedrine | 20 | −0.852 | 0.262 | −0.875 | −0.687 | 0.210 | 0.554 | −0.075 | −0.171 | 0.130 | 1000118 | 11489119 |
| retinol | 20 | 0.969 | 0.236 | −0.374 | 0.314 | −0.098 | −0.791 | −0.028 | −0.119 | 0.167 | 1000121 | 11489266 |
| cinchonidine | 20 | 0.224 | −0.165 | 0.142 | −0.515 | 1.788 | −0.839 | 0.395 | 0.392 | 0.304 | 1000122 | 11488535 |
| triamcinolone diacetate | 20 | 0.245 | −0.873 | 0.334 | −0.048 | −0.125 | −0.519 | 0.272 | 0.003 | 0.751 | 1000123 | 11488775 |
| atropine sulfate | 13.82 | −1.258 | 1.555 | −1.107 | −0.388 | 0.338 | 0.071 | 0.362 | 0.481 | 0.057 | 1000124 | 11467713 |
| atropine | 20 | 0.144 | −0.142 | −0.973 | −0.261 | 1.677 | 1.709 | −0.046 | −0.072 | −0.036 | 1000124 | 11487910 |
| chenodiol | 20 | −0.576 | 0.331 | −1.535 | −0.682 | 1.400 | 0.378 | 0.112 | −0.055 | 0.470 | 1000126 | 11488430 |
| triamcinolone acetonide | 20 | −1.108 | −0.121 | −0.645 | −0.532 | −0.589 | −0.827 | −1.180 | −1.318 | −0.680 | 1000127 | 11488659 |
| carbenoxolone | 20 | −0.985 | 1.241 | −0.858 | −0.952 | −0.097 | 1.239 | −0.334 | −0.181 | −0.600 | 1000129 | 11488767 |
| testosterone | 20 | −0.683 | 0.074 | −1.060 | 0.259 | 0.727 | −0.787 | −1.401 | −1.296 | −1.306 | 1000133 | 11489615 |
| cytidine | 20 | 0.026 | −0.291 | −0.405 | −0.557 | −0.229 | 0.658 | 0.492 | 0.547 | 0.354 | 1000134 | 11488977 |
| flurbiprofen | 16.38 | −1.607 | 0.523 | −1.110 | −0.349 | 0.266 | −0.058 | −0.151 | −0.215 | 0.004 | 1000135 | 11468065 |
| flurbiprofen | 20 | −0.630 | 0.116 | −1.494 | −1.110 | 0.145 | −0.011 | 0.023 | 0.008 | 0.123 | 1000135 | 11488841 |
| equilin | 20 | −0.475 | 0.291 | −0.585 | −0.681 | 0.633 | 0.464 | 0.464 | 0.474 | 0.341 | 1000136 | 11488562 |
| ibuprofen | 20 | −0.496 | −0.246 | −1.141 | −0.112 | 0.749 | 0.425 | −0.460 | −0.408 | −0.531 | 1000138 | 11487945 |
| moxalactam | 7.68 | 0.045 | 0.978 | −0.992 | −0.516 | 0.917 | 0.101 | −0.167 | −0.104 | −0.274 | 1000139 | 11467967 |
| moxalactam | 20 | −0.223 | 0.469 | −1.248 | −0.553 | 1.021 | 0.395 | 0.344 | 0.392 | 0.257 | 1000139 | 11488883 |
| aesculin | 20 | −0.110 | 0.965 | −1.939 | −0.533 | 1.052 | 0.687 | −0.080 | 0.059 | −0.299 | 1000141 | 11488392 |
| 18alpha-glycyrrhetinic acid | 20 | 0.249 | 0.392 | −1.047 | −0.080 | 1.345 | 0.126 | 0.342 | 0.351 | 0.295 | 1000142 | 11488236 |
| mimosine | 20.18 | −0.944 | −0.168 | −0.913 | −0.593 | 0.789 | 0.344 | −0.354 | −0.293 | −0.415 | 1000143 | 11467527 |
| mimosine | 20 | −0.286 | −0.397 | −1.208 | −0.140 | 0.352 | 0.021 | −0.133 | −0.106 | −0.179 | 1000143 | 11488472 |
| levofloxacin | 20 | −0.073 | 1.037 | −0.557 | −0.435 | 0.757 | −0.071 | 0.303 | 0.352 | 0.186 | 1000155 | 11489492 |
| naproxen | 17.38 | −0.979 | 0.098 | −1.287 | −0.935 | 0.565 | 0.344 | −0.546 | −0.584 | −0.413 | 1000165 | 11467193 |
| tobramycin | 8.56 | −0.704 | −1.234 | −1.211 | −0.868 | 0.653 | 0.335 | −0.251 | −0.278 | −0.154 | 1000177 | 11467692 |
| hyoscyamine | 13.82 | −0.871 | 0.018 | −0.804 | 0.167 | 0.584 | 0.319 | −1.052 | −0.909 | −1.177 | 1000200 | 11467381 |
| (R)-propranolol | 15.42 | −0.913 | −0.657 | −0.854 | −0.406 | −0.010 | 0.018 | −0.292 | −0.450 | 0.073 | 1000206 | 11468223 |
| fusidic acid | 7.74 | −0.475 | −0.781 | −1.231 | −0.238 | 0.899 | 0.047 | −0.160 | −0.276 | 0.109 | 1000211 | 11467538 |
| urosiol | 10.18 | −0.172 | −0.402 | −0.103 | 0.058 | 0.054 | −0.412 | 0.638 | 0.447 | 0.902 | 1000212 | 11468106 |
| thyroxine | 5.14 | 0.900 | 2.161 | −0.689 | −0.714 | −0.304 | 0.694 | −0.769 | −0.424 | −1.324 | 1000219 | 11467551 |
| thyroxine | 20 | 0.805 | 1.712 | −1.486 | −0.646 | 0.259 | 1.161 | −0.838 | −0.847 | −0.628 | 1000219 | 11488389 |
| fluticasone | 8 | −1.133 | −0.825 | −0.911 | 0.243 | 0.938 | −1.313 | 0.412 | 0.220 | 0.717 | 1000221 | 11468145 |
| fludrocortisone acetate | 9.46 | 0.352 | −0.711 | −0.539 | −0.314 | −0.337 | −0.100 | −0.536 | −0.599 | −0.315 | 1000235 | 11467429 |
| flurandrenolide | 9.16 | 0.120 | 0.061 | −0.617 | 0.531 | 0.656 | 0.966 | 0.508 | 0.490 | 0.442 | 1000240 | 11467793 |
| cefotiam | 7.6 | 1.338 | 1.785 | −0.522 | −0.191 | −0.450 | 1.058 | −0.329 | −0.142 | −0.645 | 1000242 | 11467630 |
| dexamethasone acetate | 9.2 | −0.935 | −0.334 | −0.041 | 0.478 | −0.110 | −1.608 | 0.056 | −0.230 | 0.593 | 1000246 | 11467278 |
| aclacinomycin A1 | 20 | −1.848 | 1.204 | −1.542 | −1.920 | 0.586 | 0.186 | −1.653 | −1.759 | −1.166 | 1000247 | 11489750 |
| becanamycin | 20 | 0.295 | −0.371 | 0.002 | −0.339 | 0.623 | 0.195 | 0.068 | 0.170 | −0.120 | 1000253 | 11488456 |
| ethambutol | 19.58 | −0.326 | 1.132 | 0.669 | −0.448 | 1.772 | −1.231 | 0.624 | 0.518 | 0.667 | 1000260 | 11467176 |
| beclomethasone | 7.68 | −0.448 | −0.527 | −1.896 | −0.501 | 0.634 | −0.129 | −0.055 | −0.152 | 0.158 | 1000270 | 11468003 |
| bromocriptine | 6.12 | 2.123 | 0.048 | −0.409 | −0.738 | 0.237 | −0.066 | 0.150 | 0.435 | −0.491 | 1000273 | 11467269 |
| doxorubicin | 7.36 | −3.833 | −4.338 | −4.858 | −2.782 | −3.244 | −4.653 | −0.420 | −1.850 | 2.569 | 1000279 | 11467586 |
| norethindrone | 13.4 | −0.987 | 1.253 | −0.971 | −0.627 | 0.371 | 0.903 | −0.282 | −0.083 | −0.624 | 1000286 | 11467401 |
| ritodrine | 13.92 | 0.892 | −0.401 | −0.658 | −1.012 | 1.731 | −0.319 | 0.439 | 0.459 | 0.315 | 1000292 | 11467497 |
| mometasone | 7.68 | −0.544 | −0.485 | −0.899 | 0.515 | 0.261 | −0.329 | 0.624 | 0.642 | 0.457 | 1000293 | 11467720 |
| cefmetazole | 8.48 | −0.798 | −0.622 | −1.102 | −0.012 | 0.777 | 0.363 | −0.526 | −0.434 | −0.603 | 1000312 | 11467848 |
| benazepril | 20 | −0.214 | 1.249 | −1.063 | 0.132 | −0.701 | 0.877 | −0.488 | −0.246 | −0.856 | 1000322 | 11488298 |
| liothyronine | 6.14 | 0.478 | 0.874 | −1.516 | −0.507 | 1.438 | −0.017 | −0.090 | −0.029 | −0.200 | 1000323 | 11468001 |
| liothyronine | 20 | −0.337 | 0.371 | −0.990 | −0.267 | 0.645 | −0.118 | −1.581 | −1.758 | −0.963 | 1000323 | 11489800 |
| strophantine | 6.84 | 0.897 | 0.392 | 0.489 | −0.432 | −0.336 | 0.295 | −0.500 | −0.170 | −1.073 | 1000325 | 11467619 |
| dibekacin | 20 | 1.367 | 0.050 | −1.282 | 0.289 | 0.027 | 0.464 | −0.603 | −0.522 | −0.645 | 1000338 | 11489344 |
| cephalexin | 11.52 | −1.163 | 0.555 | −0.897 | −0.568 | 1.140 | 0.140 | 0.062 | 0.000 | 0.178 | 1000342 | 11467506 |
| dextromethorphan | 14.74 | −1.339 | 0.408 | −0.580 | −0.583 | 1.381 | 0.106 | 0.216 | 0.202 | 0.209 | 1000343 | 11467507 |
| meropenem | 10.44 | −0.017 | −0.578 | −0.190 | −0.198 | −0.091 | −0.519 | 0.773 | 0.732 | 0.697 | 1000348 | 11468254 |
| rosuvastatin | 20 | −0.298 | 0.668 | −0.858 | −0.713 | 0.573 | −0.198 | −0.017 | −0.175 | 0.377 | 1000377 | 11488906 |
| almotriptan | 20 | 0.044 | 0.944 | −1.755 | −0.188 | 0.388 | 0.905 | −0.888 | −0.674 | −1.113 | 1000393 | 11488314 |
| tegaserod | 20 | −0.414 | 0.226 | −0.521 | −0.148 | 0.945 | 0.003 | −0.339 | −0.278 | −0.321 | 1000411 | 11488916 |
| atovaquone | 20 | 0.141 | −0.954 | −1.839 | −0.627 | −0.475 | −0.263 | 0.775 | 0.478 | 1.282 | 1000656 | 11489481 |
| teniposide | 20 | −3.245 | −5.758 | −4.575 | −3.373 | −1.526 | −3.537 | −1.760 | −2.625 | 0.375 | 1000697 | 11489463 |
| cyclizine | 15.02 | 0.498 | 0.836 | −0.248 | −0.355 | 1.001 | 0.646 | −0.412 | −0.325 | −0.515 | 1000807 | 11467658 |
| cyclizine | 20 | 0.072 | 1.230 | −0.487 | −0.793 | 0.711 | 0.061 | −1.114 | −0.822 | −1.428 | 1000807 | 11488990 |
| miglitol | 20 | −0.485 | 0.607 | −1.538 | −0.529 | 1.288 | 0.148 | −0.187 | −0.315 | 0.158 | 1000878 | 11488323 |
| laudanosine | 11.2 | −0.259 | 0.002 | −0.873 | −0.944 | −0.038 | 0.092 | 0.056 | −0.021 | 0.190 | 1000946 | 11467739 |
| laudanosine | 20 | −0.500 | 0.558 | −0.349 | −0.779 | 0.292 | 0.873 | −1.200 | −0.885 | −1.619 | 1000946 | 11488479 |
| valdecoxib | 20 | 0.658 | 2.260 | −0.932 | 0.408 | 1.164 | −1.335 | −0.348 | −0.381 | −0.168 | 1001030 | 11488324 |
| avobenzone | 20 | −0.230 | 0.492 | −0.339 | −0.129 | 0.116 | 0.098 | −0.816 | −0.637 | −0.979 | 1001204 | 11489479 |
| dactinomycin | 20 | −3.297 | −4.046 | −4.712 | −2.545 | −2.743 | −4.013 | 1.193 | −0.575 | 4.709 | 1001284 | 11488251 |
| diphemanil | 14.36 | −0.579 | −0.453 | −0.489 | −0.112 | 1.826 | 0.139 | 0.376 | 0.332 | 0.361 | 1001312 | 11467227 |
| dirithromycin | 20 | −0.880 | −0.210 | −0.816 | −0.455 | 0.753 | 0.126 | −0.035 | 0.046 | −0.154 | 1001314 | 11489471 |
| trisodium ethylenediamine tetracetate | 20 | −0.946 | 0.328 | −1.123 | 0.474 | −0.436 | 0.392 | −0.539 | −0.367 | −0.829 | 1001324 | 11487819 |
| escitalopram | 20 | 1.301 | 0.837 | 1.059 | −0.425 | −0.377 | 0.752 | −0.372 | −0.037 | −0.946 | 1001332 | 11488367 |
| ezetimibe | 20 | 2.411 | 1.732 | −0.377 | 0.879 | −0.183 | −0.066 | −0.152 | −0.147 | −0.093 | 1001346 | 11488305 |
| gatifloxacin | 20 | 0.995 | 1.542 | −0.531 | −0.248 | 0.452 | 0.765 | 0.305 | 0.357 | 0.188 | 1001366 | 11488303 |
| metaxalone | 20 | 0.239 | 0.421 | −0.973 | −0.359 | 0.377 | −0.068 | 0.167 | 0.107 | 0.298 | 1001451 | 11488364 |
| monobenzone | 19.98 | −0.166 | −0.012 | −0.874 | −0.741 | 0.059 | 0.289 | −0.414 | −0.556 | −0.052 | 1001471 | 11468060 |
| olmesartan medoxomil | 20 | −0.233 | 0.783 | −1.486 | −0.603 | −0.320 | 0.612 | −0.497 | −0.468 | −0.407 | 1001491 | 11488322 |
| oxcarbazepine | 20 | 1.186 | 1.316 | −0.752 | 0.049 | 0.052 | 0.568 | −0.838 | −0.699 | −0.921 | 1001496 | 11488299 |
| perindopril erbumine | 20 | −1.461 | 1.120 | −1.318 | −0.841 | 1.294 | 0.540 | −0.410 | −0.468 | −0.132 | 1001518 | 11488924 |
| fenamisal | 20 | −0.505 | 0.548 | −1.690 | −0.495 | 1.273 | 0.492 | 0.371 | 0.145 | 0.804 | 1001523 | 11488255 |
| podophyllotoxin | 9.66 | −0.077 | −0.405 | −1.865 | −0.902 | −1.401 | −1.416 | 1.189 | 1.598 | 0.118 | 1001531 | 11467930 |
| podofilox | 20 | 0.789 | −0.716 | −1.507 | −0.212 | −1.436 | −0.487 | 2.274 | 2.534 | 1.280 | 1001531 | 11488694 |
| tannic acid | 20 | 0.979 | 1.307 | −1.257 | −4.214 | −1.385 | 0.416 | 0.778 | 0.673 | 0.881 | 1001621 | 11488359 |
| torsemide | 11.48 | −0.063 | −0.620 | −0.196 | 0.218 | −0.076 | 0.826 | −0.148 | −0.282 | 0.151 | 1001638 | 11468178 |
| torsemide | 20 | −0.180 | 0.707 | −0.787 | −0.536 | 0.679 | −0.369 | 0.055 | 0.048 | 0.111 | 1001638 | 11488958 |
| tocopherol | 9.28 | −1.090 | 0.824 | −1.285 | −0.794 | −0.564 | 0.977 | −0.714 | −0.508 | −0.996 | 1001661 | 11467552 |
| (S)-(−)-atenolol | 15.02 | 0.012 | 0.476 | −0.552 | 0.396 | −0.554 | 0.030 | −0.280 | −0.224 | −0.357 | 1001857 | 11468101 |
| (R)-(+)-atenolol | 15.02 | −0.846 | −0.351 | −1.053 | −0.573 | 1.639 | −0.033 | −0.106 | −0.170 | 0.053 | 1001858 | 11467684 |
| acetylcysteine | 20 | −0.303 | −0.172 | −1.437 | 0.809 | 0.885 | 0.402 | 1.142 | 1.107 | 0.920 | 1001897 | 11487902 |
| epicatechin | 20 | −1.657 | 0.915 | −1.727 | −2.362 | 0.513 | −0.087 | −0.903 | −0.887 | −0.771 | 1001923 | 11488491 |
| epiandrosterone | 13.78 | −1.401 | 0.361 | 0.386 | 0.515 | 0.723 | −1.183 | 0.911 | 0.882 | 0.791 | 1001924 | 11467588 |
| flupentixol | 9.2 | 1.078 | 0.634 | 0.369 | 0.364 | 1.219 | 0.933 | 0.320 | 0.188 | 0.521 | 1001939 | 11467488 |
| gelsemine | 12.4 | −0.066 | 0.190 | −1.414 | −0.678 | 1.248 | 0.289 | 0.178 | 0.274 | −0.062 | 1001945 | 11467810 |
| huperzine A | 20 | −1.231 | 0.388 | −1.723 | −0.628 | 0.302 | −0.152 | −0.618 | −0.549 | −0.653 | 1001954 | 11488651 |
| methylprednisolone, 6-alpha | 10.68 | −0.893 | −0.062 | −0.968 | 0.330 | 0.622 | −1.075 | 0.382 | 0.331 | 0.419 | 1001967 | 11467427 |
| oxprenolol | 15.08 | 0.312 | 0.436 | 0.479 | −0.842 | −0.060 | 0.047 | −0.060 | 0.047 | −0.283 | 1001977 | 11468205 |
| 1R,2S-phenylpropylamine | 20 | −0.684 | 0.610 | −1.501 | −1.027 | 0.690 | −0.340 | 1.123 | 1.182 | 0.832 | 1001994 | 11488332 |
| shikimic acid | 20 | −0.210 | −0.054 | −0.775 | −0.574 | 0.752 | −0.575 | 0.390 | 0.358 | 0.393 | 1002002 | 11489324 |
| triamcinolone | 10.14 | −1.198 | −0.030 | 0.109 | −0.005 | 0.391 | −1.483 | 0.019 | −0.199 | 0.424 | 1002008 | 11467268 |
| vigabatrin | 30.96 | 1.247 | 1.232 | −1.673 | −0.961 | 1.259 | 1.011 | −0.999 | −0.819 | −1.173 | 1002022 | 11467649 |
| zimelidine | 12.6 | −0.256 | 0.669 | −1.241 | −0.299 | 0.451 | 0.133 | −0.638 | −0.501 | −0.837 | 1002029 | 11467240 |
| perseitol | 20 | −1.138 | 0.707 | −0.276 | −0.981 | 1.184 | 0.652 | −0.400 | −0.515 | −0.054 | 1002679 | 11489572 |
| hydroxytoluic acid | 20 | −0.105 | 0.802 | 0.548 | −0.525 | 0.821 | 1.179 | 0.651 | 0.570 | 0.627 | 1002775 | 11487967 |
| phenylbutyrate | 20 | −0.302 | 0.550 | −0.567 | −0.133 | 1.253 | −0.262 | −0.397 | −0.361 | −0.359 | 1002855 | 11488326 |
| fenbutyramide | 20 | 0.675 | 1.115 | −0.675 | −0.485 | 0.163 | 0.580 | −1.233 | −1.228 | −0.971 | 1002856 | 11488308 |
| thymoquinone | 20 | −0.503 | 0.856 | −0.212 | −0.361 | 0.641 | −0.765 | −0.569 | −0.501 | −0.617 | 1003215 | 11488516 |
| eudesmic acid | 20 | −0.436 | 0.802 | −0.440 | −0.769 | 0.640 | 0.841 | −0.405 | −0.402 | −0.341 | 1003514 | 11488607 |
| phenylacetohydroxamic acid | 20 | −0.682 | −0.170 | −1.861 | −0.849 | 0.883 | 0.136 | 0.062 | 0.055 | 0.101 | 1003535 | 11489532 |
| larixinic acid | 20 | −0.702 | 0.333 | −1.835 | −0.810 | 0.179 | 0.132 | −1.054 | −1.119 | −0.673 | 1003823 | 11489611 |
| N-methylanthranilic acid | 20 | −0.146 | −0.082 | −0.120 | −1.181 | 0.967 | 0.299 | −0.385 | −0.179 | −0.683 | 1004713 | 11489732 |
| metacetamol | 20 | −1.338 | 0.154 | −2.107 | −0.578 | 0.692 | 0.182 | −0.259 | −0.114 | −0.460 | 1004889 | 11488333 |
| benzanthrone | 20 | 0.277 | 0.108 | −1.435 | −1.288 | 1.609 | −0.308 | 0.690 | 0.880 | 0.155 | 1005991 | 11488582 |
| 5,7-dihydroxy-4-methylcoumarin | 20 | −0.483 | 1.206 | −0.741 | −1.609 | 0.687 | 0.189 | 0.525 | 0.710 | 0.052 | 1006104 | 11489172 |
| purpurin | 20 | −1.205 | −0.402 | −2.248 | −2.389 | 1.584 | 1.010 | 0.375 | 0.336 | 0.331 | 1007083 | 11487853 |
| chrysanthemic acid | 20 | −0.582 | 0.602 | −0.735 | −0.366 | −0.121 | 0.675 | −0.437 | −0.489 | −0.222 | 1007364 | 11489607 |
| thonzonium bromide | 7.82 | −1.864 | −2.454 | −1.328 | −0.236 | −1.390 | −1.152 | 0.868 | 0.997 | 0.413 | 1007994 | 11468073 |
| pentylenetetrazole | 28.94 | 1.091 | 1.642 | −1.013 | −0.228 | 0.069 | 1.064 | −0.462 | −0.422 | −0.495 | 1008060 | 11467310 |
| pentetrazole | 20 | 0.523 | 0.164 | −0.423 | 0.010 | −0.535 | 0.949 | 0.382 | 0.373 | 0.386 | 1008060 | 11488937 |
| diffratic acid | 20 | 0.620 | 1.210 | −2.807 | 0.281 | 1.373 | 0.318 | 0.959 | 0.666 | 1.344 | 1008178 | 11488546 |
| dibenzoylmethane | 20 | −0.350 | −1.193 | −1.719 | −0.031 | 0.846 | −0.484 | −0.553 | −0.956 | 0.330 | 1008492 | 11487854 |
| O-veratraldehyde | 20 | −1.299 | −1.020 | −2.796 | 0.347 | −0.925 | −1.483 | −0.478 | −0.568 | −0.245 | 1008535 | 11489780 |
| mandelic acid, methyl ester | 20 | 0.277 | −0.088 | −0.364 | −0.284 | 0.074 | 0.835 | −0.312 | −0.147 | −0.650 | 1008719 | 11487998 |
| alloxan | 20 | 0.483 | 1.409 | −0.552 | −0.634 | 0.080 | 0.678 | 0.373 | 0.362 | 0.358 | 1009258 | 11488347 |
| alizarin | 20 | −0.562 | 1.648 | −0.981 | −2.407 | 1.154 | 0.211 | 0.600 | 0.667 | 0.401 | 1009294 | 11488213 |
| hematein | 20 | −0.357 | −0.036 | −1.726 | −1.215 | 0.835 | 0.132 | −0.042 | −0.084 | 0.086 | 1009367 | 11488428 |
| veratric acid | 20 | 0.049 | −0.216 | −1.025 | −0.545 | 0.049 | −0.013 | 0.147 | 0.295 | −0.111 | 1009654 | 11488898 |
| anthraquinone | 20 | 0.201 | 0.593 | −0.987 | 0.865 | 0.859 | 0.975 | −0.476 | −0.435 | −0.431 | 1009851 | 11488221 |
| mucic acid | 20 | −0.614 | 0.362 | −1.136 | −0.633 | −0.025 | 0.379 | −0.939 | −1.022 | −0.592 | 1009973 | 11489270 |
| chloranil | 20 | −4.795 | −8.532 | −6.360 | −4.051 | −3.428 | −5.311 | −0.841 | −2.448 | 2.504 | 1010201 | 11487840 |
| diphenylurea | 20 | 1.065 | −1.101 | 0.770 | −0.598 | −0.022 | −0.392 | 0.472 | 0.423 | 0.510 | 1010251 | 11489654 |
| lawsone | 20 | −0.304 | 0.103 | −0.834 | −0.260 | −0.103 | −0.273 | 0.227 | 0.240 | 0.163 | 1010348 | 11489322 |
| brazilin | 20 | −1.426 | 1.655 | −1.218 | −0.602 | −1.481 | −0.848 | −0.672 | 0.001 | −1.884 | 1010376 | 11488198 |
| haematoxylin | 20 | −1.044 | 0.382 | −1.335 | −1.035 | 0.225 | 0.028 | 0.094 | 0.337 | −0.395 | 1010377 | 11488415 |
| coumarin | 20 | −0.167 | 0.494 | −0.805 | −0.518 | 0.740 | 0.232 | −0.132 | −0.217 | 0.011 | 1010471 | 11488119 |
| trichlorfon | 15.54 | 1.232 | 0.239 | −1.804 | −0.493 | −0.740 | 0.081 | −1.085 | −0.845 | −1.404 | 1010605 | 11467199 |
| apiole | 20 | 0.028 | 0.655 | −1.184 | −0.188 | 1.294 | 0.460 | 0.591 | 0.611 | 0.470 | 1010689 | 11488235 |
| 1,4-naphthoquinone | 20 | −5.494 | −8.116 | −6.183 | −3.902 | −3.491 | −4.549 | −2.220 | −2.490 | −1.260 | 1011006 | 11488297 |
| apomorphine | 20 | −0.852 | −1.905 | −2.279 | −1.416 | −0.457 | 0.044 | 0.302 | 0.401 | −0.037 | 1011303 | 11487958 |
| 4-methylesculetin | 20 | −0.301 | 0.901 | −2.063 | −0.362 | 1.255 | 0.262 | −0.300 | −0.190 | −0.430 | 1011559 | 11488433 |
| tryptophan | 20 | −0.209 | 0.143 | −1.175 | −0.878 | 0.517 | 0.200 | 0.239 | 0.190 | 0.360 | 1012497 | 11488918 |
| butylparaben | 20.6 | −0.445 | 1.180 | −0.832 | −0.442 | 0.648 | 0.379 | −1.142 | −1.062 | −1.095 | 1012530 | 11468042 |
| norcantharidin | 20 | −0.657 | −0.697 | −3.125 | 0.478 | 0.510 | −1.419 | −0.399 | −0.196 | −0.699 | 1013144 | 11489499 |
| adenine | 20 | −0.236 | 0.732 | −1.384 | −0.283 | 0.030 | 0.146 | −1.374 | −1.354 | −1.114 | 1013195 | 11488399 |
| xanthone | 20 | 0.474 | −1.238 | −0.928 | −0.078 | 0.112 | 0.526 | −0.303 | −0.066 | −0.774 | 1013706 | 11487868 |
| indole-2-carboxylic acid | 20 | 0.090 | −0.861 | −0.726 | −0.283 | −0.079 | 0.237 | 0.464 | 0.549 | 0.149 | 1014792 | 11487837 |
| D-arabitol | 20 | −1.158 | −0.213 | −0.597 | −0.705 | 0.316 | 0.521 | −1.273 | −1.307 | −0.907 | 1014978 | 11489562 |
| adonitol | 20 | −0.469 | −0.242 | −1.425 | −0.761 | 0.234 | 0.850 | −0.211 | −0.210 | −0.128 | 1014978 | 11489610 |
| gramine | 22.96 | −0.958 | −0.640 | −0.947 | −0.451 | 0.657 | −0.212 | 0.176 | −0.333 | 1.191 | 1014994 | 11467777 |
| xanthoxylin | 20 | −1.210 | 0.423 | −1.069 | −0.651 | 0.420 | 0.269 | 0.110 | 0.131 | 0.085 | 1015281 | 11488279 |
| riboflavin | 10.62 | 0.029 | 0.364 | −1.380 | −0.767 | 1.150 | 0.109 | −0.002 | 0.195 | −0.388 | 1015808 | 11467782 |
| aminolevulinic acid | 20 | −0.207 | 0.354 | −0.846 | −0.065 | 0.035 | 0.213 | −0.105 | 0.030 | −0.308 | 1015899 | 11489478 |
| rhamnetin | 20 | −1.211 | 1.672 | −0.975 | 0.717 | 0.228 | 1.561 | −0.657 | −0.304 | −1.273 | 1016582 | 11488458 |
| gallic acid | 20 | −1.517 | −0.490 | −1.991 | −1.311 | 0.281 | −1.678 | 0.102 | 0.279 | −0.224 | 1016781 | 11488215 |
| diallyl sulfide | 20 | −1.284 | −0.397 | −1.776 | −0.930 | 1.047 | −0.217 | 0.210 | 0.264 | 0.047 | 1017172 | 11488653 |
| 6-aminonicotinamide | 20 | −0.531 | −1.410 | −0.525 | 0.082 | 0.871 | 0.520 | −0.386 | −0.355 | −0.340 | 1017318 | 11489525 |
| osajin | 20 | −1.424 | −2.997 | −2.351 | −2.214 | 2.139 | −2.625 | −1.054 | −0.943 | −1.128 | 1017609 | 11487991 |
| phenformin | 19.48 | 0.372 | −0.240 | −1.696 | −0.517 | −0.545 | 0.241 | 0.616 | 0.870 | −0.059 | 1018627 | 11467327 |
| 2,6-dimethoxyquinone | 20 | −5.784 | −7.615 | −5.944 | −3.903 | −4.010 | −3.043 | −1.600 | −2.620 | 0.860 | 1019365 | 11488597 |
| 2-methyl gramine | 20 | −2.419 | −2.788 | −2.058 | 0.371 | −1.879 | −3.013 | −0.685 | −0.660 | −0.616 | 1019607 | 11488506 |
| methylatropine | 13.14 | 0.416 | 0.651 | 1.098 | 0.249 | 1.762 | −0.661 | 0.508 | 0.407 | 0.603 | 1019709 | 11468048 |
| homochlorcyclizine | 12.7 | −0.348 | −0.769 | −1.521 | −0.629 | 0.579 | −0.789 | −0.644 | −0.711 | −0.387 | 1019722 | 11467431 |
| metameconine | 20 | 0.050 | −0.214 | −1.318 | −0.277 | 0.658 | 0.486 | −0.728 | −0.437 | −1.128 | 1019872 | 11489718 |
| phenacylamine | 20 | −0.497 | 0.572 | −1.279 | −0.234 | 0.384 | 0.088 | −1.272 | −1.327 | −0.965 | 1019888 | 11489809 |
| benzylhydrazine | 20 | −0.105 | 1.031 | −1.343 | −0.316 | 0.768 | 0.786 | −0.681 | −0.342 | −1.160 | 1020088 | 11489039 |
| esculetin | 22.46 | −1.108 | −0.015 | 0.611 | 0.129 | −0.636 | 0.134 | −0.568 | −0.501 | −0.609 | 1020463 | 11468088 |
| esculetin | 20 | −0.918 | 0.401 | −1.955 | 0.086 | 0.194 | −1.251 | −0.984 | −0.793 | −1.149 | 1020463 | 11488402 |
| alpha-mangostin | 20 | 0.984 | 0.277 | 0.526 | −2.090 | 1.261 | −1.053 | −0.012 | −0.097 | 0.197 | 1020994 | 11489436 |
| ethamsylate | 21.04 | −1.122 | −0.260 | −0.708 | −0.824 | 0.970 | 0.142 | 0.117 | 0.229 | −0.159 | 1022844 | 11468163 |
| 3-acetylcoumarin | 21.26 | 0.165 | 1.143 | −0.293 | −0.177 | −0.010 | 0.650 | 0.394 | 0.502 | 0.085 | 1022907 | 11468039 |
| osthol | 20 | 1.353 | 1.846 | −0.371 | 1.293 | 0.745 | 0.452 | −0.599 | −0.248 | −1.221 | 1023016 | 11488539 |
| carbarsone | 15.38 | 0.601 | 0.661 | −1.304 | −0.622 | −0.236 | 0.575 | 0.175 | 0.128 | 0.241 | 1023517 | 11467561 |
| 4-hydroxy-6-methylpyran-2-one | 20 | −0.952 | 0.095 | −1.112 | −0.736 | 0.314 | −0.316 | 0.182 | 0.294 | −0.135 | 1024489 | 11489794 |
| 6,7-dimethoxy-1-methyl-1,2,3,4- | 19.3 | −0.146 | −0.022 | −1.532 | −0.719 | 0.831 | 0.319 | 0.168 | 0.197 | 0.080 | 1024517 | 11467680 |
| tetrahydroisoquinoline | | | | | | | | | | | | |
| salsolidine | 20 | −0.667 | 0.104 | −1.413 | −0.706 | 0.269 | −0.007 | −0.837 | −0.765 | −0.779 | 1024517 | 11489442 |
| (D,L)-tetrahydroberberine | 11.78 | 0.186 | −1.024 | −2.007 | −0.541 | −0.601 | 0.408 | 0.475 | 0.372 | 0.572 | 1025381 | 11467820 |
| 2-aminobenzenesulfonamide | 23.22 | 0.130 | 0.688 | −0.277 | −0.580 | 0.280 | 0.044 | 0.721 | 0.549 | 0.921 | 1025776 | 11468061 |
| chrysophanol | 20 | −0.004 | 0.285 | −2.906 | −1.225 | 0.291 | 0.561 | −1.273 | −1.181 | −1.271 | 1025940 | 11487859 |
| 2-hydroxy-3,4-dimethoxybenzoic acid | 20 | −1.147 | 1.210 | −0.857 | 0.908 | 0.261 | 0.331 | −1.003 | −1.020 | −0.832 | 1026119 | 11489737 |
| 2-acetylpyrrole | 20 | −0.920 | 0.397 | −1.050 | −0.692 | 0.716 | −0.009 | −0.122 | −0.067 | −0.253 | 1029168 | 11489772 |
| safrolglycol | 20 | −0.657 | 0.236 | −1.436 | −0.947 | 0.344 | −0.051 | −0.539 | −0.367 | −0.796 | 1029487 | 11488499 |
| 2,6-dihydroxy-4-methoxytoluene | 20 | −0.805 | 0.068 | −1.048 | −0.565 | 0.139 | 0.267 | −0.918 | −0.887 | −0.752 | 1029490 | 11489619 |
| moroxidine | 23.36 | −0.140 | 0.676 | −0.643 | −0.722 | −0.040 | 1.127 | −0.450 | −0.245 | −0.819 | 1029858 | 11467232 |
| tropine | 28.32 | −1.554 | 0.953 | −0.558 | −0.370 | −0.114 | −0.175 | 0.244 | 0.350 | −0.030 | 1029881 | 11468225 |
| 4-methyldaphnetin | 20 | −1.167 | 0.765 | −1.183 | −0.519 | −1.229 | 0.445 | −0.735 | −0.866 | −0.293 | 1030891 | 11488137 |
| citrulline | 20 | −0.129 | 1.090 | −1.207 | 0.077 | 0.307 | 0.910 | 0.449 | 0.509 | 0.230 | 1031375 | 11489187 |
| 4-acetoxyphenol | 20 | −0.142 | 0.982 | −0.987 | −1.179 | −0.565 | −0.347 | −0.615 | −0.687 | −0.286 | 1031842 | 11488961 |
| cresopyrine | 20 | 0.505 | 0.240 | −0.635 | −0.345 | −0.146 | 0.617 | 0.111 | 0.041 | 0.279 | 1032444 | 11488371 |
| flavanone | 20 | −1.078 | −0.294 | −0.639 | −0.733 | −0.221 | −0.102 | −0.483 | −0.591 | −0.224 | 1032994 | 11488021 |
| tangeritin | 20 | 1.230 | −0.141 | −0.567 | 0.380 | 1.163 | 0.951 | −0.302 | −0.228 | −0.355 | 1034727 | 11489514 |
| harmane | 21.96 | −0.359 | −0.385 | −1.138 | 0.074 | 1.285 | −0.307 | −0.324 | −0.334 | −0.227 | 1035065 | 11467768 |
| phloracetophenone | 20 | −0.384 | −0.673 | −0.760 | −0.710 | 1.330 | 0.059 | −0.393 | −0.613 | 0.087 | 1035250 | 11489805 |
| 3-hydroxyflavone | 20 | −5.146 | −6.482 | −4.941 | −2.910 | −3.665 | −4.110 | −2.484 | −3.582 | 0.181 | 1036721 | 11489208 |
| pseudopelletierine | 26.1 | −0.946 | 0.266 | −1.231 | −0.356 | 0.441 | 0.326 | 0.171 | −0.085 | 0.668 | 1039072 | 11467773 |
| 3-acetamidocoumarin | 19.68 | 0.050 | 2.085 | 0.646 | 0.367 | −0.180 | 0.627 | 0.143 | 0.304 | −0.226 | 1040327 | 11468117 |
| 3-methoxycatechol | 20 | −1.592 | −0.649 | −1.577 | −1.022 | −1.153 | −0.541 | −1.331 | −1.126 | −1.437 | 1040795 | 11489733 |
| orthothymotinic acid | 20 | −1.810 | −0.013 | −1.403 | −0.822 | 1.518 | 0.397 | 0.067 | −0.220 | 0.693 | 1041649 | 11488254 |
| harmol | 20.18 | −0.149 | −0.557 | −2.086 | −0.469 | 0.651 | 1.084 | −0.770 | −0.801 | −0.558 | 1043296 | 11467760 |
| harmol | 20 | −0.412 | 0.713 | −2.550 | 0.020 | −0.017 | 0.409 | −0.121 | −0.506 | 0.738 | 1043296 | 11488320 |
| 3-hydroxycoumarin | 20 | −0.257 | 0.054 | −1.305 | −0.084 | −0.551 | 0.288 | −0.026 | 0.031 | −0.156 | 1044412 | 11488621 |
| 5-chloroindole-2-carboxylic acid | 20 | −1.114 | 0.601 | −1.288 | −0.628 | 0.053 | 0.477 | −0.573 | −0.745 | −0.072 | 1044852 | 11488329 |
| diperodon | 10.06 | 0.447 | −0.347 | −0.253 | −0.279 | 1.342 | −0.532 | −0.681 | −0.538 | −0.832 | 1045066 | 11467448 |
| djenkolic acid | 20 | −0.063 | 0.238 | −0.410 | −0.248 | 0.019 | −0.003 | −0.940 | −0.811 | −0.971 | 1045072 | 11489605 |
| nobiletin | 20 | 1.191 | 0.037 | −1.693 | 0.397 | 0.826 | 0.396 | 1.029 | 1.252 | 0.417 | 1045397 | 11489513 |
| norharman | 20 | 0.277 | 1.571 | −1.235 | −0.056 | 1.683 | −0.972 | 0.359 | 0.502 | 0.055 | 1048361 | 11488404 |
| 6-methoxyharmalan | 18.66 | 0.431 | −0.280 | −0.542 | −0.687 | 0.951 | 0.448 | 0.030 | −0.083 | 0.253 | 1048750 | 11467769 |
| stictic acid | 20 | 0.165 | −0.832 | −1.355 | −0.780 | 1.202 | 0.938 | 0.547 | 0.809 | −0.163 | 1049466 | 11488123 |
| atranorin | 20 | −1.280 | −1.384 | −1.246 | −0.614 | 0.608 | 0.873 | −0.474 | −0.617 | −0.046 | 1049467 | 11489565 |
| asarylaldehyde | 20 | −0.477 | 0.432 | −0.959 | −0.981 | 0.951 | 0.429 | 1.067 | 1.084 | 0.888 | 1050335 | 11488202 |
| ononetin | 20 | 0.642 | 0.382 | −0.196 | −0.079 | 0.130 | −0.250 | 0.367 | 0.547 | −0.089 | 1050602 | 11488624 |
| 1,3,5-trimethoxybenzene | 20 | −0.674 | 0.179 | −2.053 | −0.641 | 2.302 | 0.545 | −0.387 | −0.307 | −0.432 | 1050711 | 11488242 |
| psoromic acid | 20 | 0.147 | −1.096 | −1.499 | −0.734 | −0.599 | 0.397 | −1.114 | −1.069 | −1.037 | 1051460 | 11488030 |
| salsoline | 20 | −0.350 | 0.448 | −0.467 | −0.964 | 0.862 | −0.470 | 0.335 | 0.145 | 0.694 | 1052338 | 11488356 |
| oxalamine | 16.3 | −0.255 | −0.739 | −1.880 | −0.635 | 1.404 | 0.631 | −0.509 | −0.379 | −0.686 | 1052436 | 11467974 |
| visnagin | 20 | −0.893 | −0.777 | −1.716 | −0.509 | 0.098 | −0.163 | 0.231 | 0.116 | 0.463 | 1052459 | 11489612 |
| quercetin tetramethyl ether | 20 | 0.552 | 1.402 | −0.354 | 1.428 | 0.442 | 0.181 | 0.147 | 0.331 | −0.277 | 1053058 | 11488527 |
| 3-hydroxy-3′,4′-dimethoxyflavone | 20 | −0.631 | −0.318 | −2.254 | 2.225 | −0.579 | −0.230 | −0.956 | −0.845 | −0.968 | 1053060 | 11489518 |
| azapropazone | 13.32 | −0.340 | −0.385 | −1.289 | −0.533 | 1.003 | 0.418 | −0.064 | −0.094 | 0.005 | 1053328 | 11468151 |
| eupatorin | 20 | −1.135 | 0.603 | −1.919 | −0.552 | 1.569 | 0.347 | −0.796 | −0.722 | −0.751 | 1054271 | 11488272 |
| evoxine | 11.52 | −0.309 | 0.694 | −1.383 | −1.248 | 1.984 | 0.144 | −0.117 | −0.110 | −0.113 | 1054504 | 11467813 |
| evoxine | 20 | −0.487 | −0.535 | −1.076 | −0.029 | 0.688 | 0.145 | −0.021 | 0.107 | −0.239 | 1054504 | 11489441 |
| skimmianine | 15.42 | −0.273 | 0.728 | −0.365 | −0.046 | 1.278 | −1.263 | −0.279 | −0.279 | −0.221 | 1054505 | 11467816 |
| ornidazole | 18.22 | 0.769 | 0.204 | −0.577 | −0.509 | 0.241 | 0.878 | −0.478 | −0.278 | −0.833 | 1054660 | 11467312 |
| lobelanidine | 11.78 | −0.257 | 0.582 | −1.552 | −1.028 | 0.549 | 0.816 | 0.048 | −0.045 | 0.222 | 1054667 | 11467730 |
| coralyne | 10.98 | 0.005 | 0.814 | −0.101 | −2.821 | 0.173 | 0.283 | −0.968 | −0.630 | −1.490 | 1055132 | 11467579 |
| coralyne | 20 | −1.233 | 0.221 | −2.193 | −2.677 | 0.998 | 0.451 | −0.286 | −0.233 | −0.311 | 1055132 | 11488421 |
| 3-hydroxy-DL-kynurenine | 17.84 | 0.692 | 0.967 | −0.405 | −0.472 | −0.323 | 0.355 | 0.140 | 0.292 | −0.205 | 1055159 | 11467599 |
| pterin-6-carboxylic acid | 20 | 0.712 | 0.141 | −0.742 | −0.307 | 0.146 | 0.484 | 0.124 | 0.349 | −0.316 | 1055442 | 11489637 |
| calycanthine | 11.54 | −0.517 | 0.362 | −1.454 | −1.253 | 0.523 | 0.347 | 0.112 | 0.078 | 0.157 | 1056553 | 11467743 |
| macluroxanthone | 20 | −2.337 | −5.271 | −3.386 | −3.197 | −2.521 | −2.511 | −2.766 | −2.306 | −3.136 | 1057125 | 11488247 |
| cyclopenthiazide | 10.52 | −1.155 | −0.565 | −0.429 | −0.719 | 0.728 | −0.087 | 0.614 | 0.653 | 0.410 | 1057366 | 11468142 |
| 3-desmethyl-5-deshydroxyscleroin | 20 | −0.001 | 0.557 | −0.927 | 0.336 | 1.578 | −0.316 | −0.723 | −0.828 | −0.429 | 1059133 | 11488006 |
| quercetin pentamethyl ether | 20 | −1.273 | −0.637 | −1.039 | −0.734 | 0.307 | −0.112 | −0.981 | −0.814 | −1.088 | 1060118 | 11489620 |
| cephalotaxine | 20 | −0.289 | 0.588 | −1.758 | −0.937 | 0.357 | 0.296 | −0.741 | −0.760 | −0.522 | 1064620 | 11488391 |
| N-acetylaspartic acid | 22.84 | 0.285 | 0.133 | −0.942 | −0.433 | 0.365 | 0.638 | −0.394 | −0.222 | −0.674 | 1064663 | 11467563 |
| albizziine | 20 | −0.570 | −0.117 | −1.129 | −0.196 | 0.966 | 0.396 | −0.230 | −0.333 | 0.075 | 1065857 | 11488275 |
| niridazole | 18.68 | −0.675 | −0.149 | −0.505 | −0.223 | 0.114 | −0.531 | −0.478 | −0.561 | −0.206 | 1067495 | 11467617 |
| orsellinic acid, ethyl ester | 20 | −0.154 | 0.432 | −1.081 | −0.109 | 0.973 | −0.722 | 0.444 | 0.207 | 0.886 | 1071570 | 11488206 |
| kainic acid | 20 | −1.056 | 0.683 | −1.319 | −0.892 | 1.417 | 0.379 | −0.128 | −0.130 | −0.022 | 1072288 | 11489064 |
| denatonium | 12.28 | −0.620 | 1.887 | −0.109 | 0.811 | −0.165 | 1.822 | −0.028 | −0.066 | 0.037 | 1073908 | 11468109 |
| homosalate | 15.24 | −0.562 | 0.298 | 0.370 | 0.753 | −0.480 | −1.151 | 0.436 | 0.372 | 0.469 | 1076027 | 11468238 |
| synephrine | 23.92 | −0.879 | −0.555 | −0.367 | 0.030 | 0.028 | −0.727 | −0.297 | −0.489 | 0.143 | 1076620 | 11468236 |
| tiletamine | 17.92 | −0.944 | −0.269 | −0.859 | −0.227 | 0.199 | 0.143 | 0.380 | 0.315 | 0.437 | 1077199 | 11468170 |
| benperidol | 10.48 | 1.359 | 0.963 | −0.768 | −0.233 | 0.541 | 0.787 | 0.044 | 0.153 | −0.186 | 1077918 | 11467632 |
| azaperone | 12.22 | 0.447 | −0.125 | 0.603 | −0.221 | −0.077 | −0.184 | 1.495 | 1.429 | 1.323 | 1078453 | 11468265 |
| azaperone | 20 | −0.674 | 0.122 | −0.529 | 0.011 | 0.829 | −0.129 | −0.600 | −0.486 | −0.633 | 1078453 | 11489066 |
| 4-hydroxyantipyrine | 19.58 | −0.437 | 0.266 | 0.102 | −0.265 | 0.948 | −0.045 | 0.934 | 0.863 | 0.852 | 1079457 | 11467178 |
| enilconazole | 13.46 | −0.521 | 3.446 | −0.330 | 1.113 | 1.262 | 0.594 | −0.326 | −0.223 | −0.488 | 1081653 | 11468111 |
| betamipron | 20 | −1.385 | −0.653 | −2.165 | −0.788 | 0.587 | 0.275 | −0.165 | −0.296 | 0.173 | 1082254 | 11488250 |
| dehydrorotenone | 20 | −0.381 | 0.836 | −1.046 | 0.884 | 0.804 | −0.674 | 0.452 | 0.554 | 0.118 | 1082584 | 11489746 |
| palmatine | 11.36 | 0.142 | 0.416 | −0.898 | −1.653 | 0.543 | 0.129 | 0.664 | 0.250 | 1.379 | 1084508 | 11467727 |
| palmatine | 20 | −1.219 | 0.461 | −1.002 | −1.380 | 1.352 | 0.145 | 0.104 | −0.242 | 0.838 | 1084508 | 11488424 |
| isopimpinellin | 20 | 0.191 | −0.582 | −0.707 | 0.188 | 0.846 | 0.230 | 0.091 | −0.190 | 0.580 | 1086162 | 11488124 |
| ethyl 1-benzyl-3-hydroxy-2-oxo[5h]pyrrole-4- | 20 | 0.379 | 0.141 | 0.969 | −0.204 | 0.154 | 0.194 | −0.659 | −0.276 | −1.273 | 1087705 | 11489647 |
| carboxylate | | | | | | | | | | | | |
| chloropyramine | 13.8 | 1.140 | 1.585 | −0.445 | −0.970 | −0.336 | 1.386 | 0.070 | 0.212 | −0.235 | 1088922 | 11467955 |
| nimustine | 20 | 0.321 | 0.010 | −0.349 | −0.211 | −0.345 | −0.429 | 0.203 | 0.345 | −0.148 | 1089854 | 11488693 |
| amidopyrine | 17.3 | 0.691 | 0.989 | −0.051 | −0.496 | −0.776 | 0.748 | 0.388 | 0.531 | −0.016 | 1090166 | 11467236 |
| lecanoric acid | 20 | 0.847 | −0.316 | −0.302 | −0.852 | −0.075 | 0.690 | −0.616 | −0.512 | −0.770 | 1090422 | 11488027 |
| physcion | 20 | −0.196 | 1.210 | −1.100 | −0.413 | 0.033 | 0.978 | −0.831 | −0.967 | −0.352 | 1090658 | 11488319 |
| clopidol | 20 | −0.449 | 1.053 | −1.395 | −0.542 | 1.696 | 0.448 | −0.313 | −0.107 | −0.716 | 1090918 | 11487834 |
| aminopterin | 20 | −0.825 | 0.979 | −1.547 | −1.020 | 0.719 | 0.484 | −1.396 | −0.989 | −1.963 | 1091345 | 11488709 |
| rhetsinine | 20 | 0.348 | −0.961 | −0.895 | 0.093 | 0.452 | 0.068 | −0.025 | 0.218 | −0.479 | 1091368 | 11489477 |
| acetopromazine | 12.26 | 0.557 | 0.746 | −1.188 | −0.923 | 0.262 | 0.844 | 0.080 | 0.182 | −0.142 | 1092107 | 11467724 |
| 4-methoxydalbergione | 20 | 0.495 | 0.730 | −0.789 | −0.797 | −0.527 | 0.113 | −0.989 | −0.932 | −0.917 | 1092958 | 11488470 |
| N-acetylproline | 20 | −0.198 | 0.707 | −1.007 | −0.132 | 0.241 | 0.493 | −0.838 | −0.635 | −1.129 | 1094519 | 11487829 |
| methacholine | 24.96 | −1.105 | −0.595 | −0.798 | 0.334 | 0.966 | −0.572 | −0.717 | −0.666 | −0.687 | 1094620 | 11467907 |
| ocadecylphosphocholine | 20 | −0.414 | −0.842 | −0.206 | −0.631 | 0.165 | −1.201 | −0.664 | −0.516 | −0.831 | 1095029 | 11489305 |
| fluoxetine | 12.94 | 0.060 | −0.919 | −1.384 | −0.269 | 0.705 | −3.551 | −0.659 | −0.480 | −0.895 | 1095093 | 11467659 |
| fluoxetine | 20 | −2.466 | −5.742 | −3.524 | 2.634 | −1.363 | −0.156 | −0.258 | −0.302 | −0.082 | 1095093 | 11489474 |
| triadimefon | 20 | −0.420 | 0.051 | −1.650 | −0.530 | 0.592 | 0.286 | −0.142 | −0.198 | 0.047 | 1099044 | 11489523 |
| lonchocarpic acid | 20 | 0.014 | 0.318 | −0.950 | 0.105 | −0.633 | 0.826 | 0.049 | −0.074 | 0.315 | 1099943 | 11489578 |
| bupropion | 16.68 | −0.417 | 1.254 | −0.230 | 0.064 | 0.168 | −0.733 | −0.390 | −0.258 | −0.590 | 1101055 | 11467397 |
| bupropion | 20 | −0.492 | −1.081 | −0.855 | −0.491 | 0.660 | 0.618 | −1.654 | −1.842 | −0.911 | 1101055 | 11489475 |
| eupatoriochromene | 20 | 0.170 | 0.533 | −0.911 | −0.829 | −0.059 | 0.627 | −0.339 | −0.173 | −0.633 | 1107153 | 11488487 |
| cuneatin methyl ether | 20 | 0.399 | 0.893 | −0.485 | 1.153 | −0.034 | 0.951 | −0.796 | −0.785 | −0.619 | 1109169 | 11488217 |
| paeonol | 20 | 0.551 | 0.203 | −0.944 | −0.424 | 0.103 | 0.306 | −0.667 | −0.613 | −0.694 | 1110410 | 11489797 |
| imperatorin | 20 | −0.193 | 0.764 | −0.883 | −0.218 | 0.942 | 0.406 | 0.465 | 0.434 | 0.481 | 1111461 | 11488192 |
| 1-aminocyclobutane carboxylic acid | 20 | −1.102 | −0.120 | −1.107 | −0.706 | 0.502 | −0.335 | −0.522 | −0.578 | −0.287 | 1111718 | 11489292 |
| quinic acid | 19.68 | 0.562 | −0.028 | −0.586 | −0.500 | −0.527 | 0.011 | 1.086 | 1.010 | 1.019 | 1111897 | 11468251 |
| herniarin | 20 | 0.366 | 0.712 | −1.164 | −0.635 | 0.295 | −0.201 | 1.569 | 1.467 | 1.527 | 1112402 | 11488214 |
| pachyrrhizin | 20 | −0.749 | 1.109 | 0.278 | 1.326 | 1.322 | 0.486 | 0.586 | 0.428 | 0.827 | 1112405 | 11488226 |
| chelidonine (+) | 20 | −0.007 | −0.075 | −2.823 | −0.734 | −1.009 | −0.051 | −0.037 | −0.059 | 0.059 | 1113269 | 11488401 |
| ethamivan | 17.92 | 0.143 | 0.416 | −0.306 | 0.296 | 1.121 | −0.159 | −0.441 | −0.077 | −1.089 | 1113307 | 11467648 |
| fisetin | 20 | −0.764 | 0.456 | −0.689 | −0.893 | 0.442 | −0.825 | −0.131 | −0.202 | 0.120 | 1113841 | 11488976 |
| eugenyl benzoate | 20 | −0.673 | −0.145 | −0.867 | −0.793 | 0.752 | 0.427 | −1.399 | −1.553 | −0.770 | 1114909 | 11489721 |
| ricinine | 24.36 | −0.938 | −0.040 | −1.117 | −0.894 | 1.586 | 0.333 | −0.705 | −0.772 | −0.425 | 1115322 | 11467826 |
| perillyl alcohol | 20 | −1.426 | −0.312 | −1.446 | −0.071 | 0.324 | −0.770 | 0.032 | 0.205 | −0.352 | 1117560 | 11488654 |
| fraxetin | 20 | −0.954 | 0.001 | −0.257 | −1.390 | −0.472 | −0.645 | −0.248 | −0.377 | 0.108 | 1119359 | 11489455 |
| 5,7,4′-trimethoxyflavone | 20 | 1.177 | 0.878 | −0.529 | 0.162 | 1.187 | 0.690 | 0.078 | 0.107 | 0.050 | 1129781 | 11488287 |
| pirlindole | 17.68 | 0.693 | 0.741 | −0.508 | 0.845 | 0.273 | 0.113 | −1.618 | −1.645 | −1.269 | 1134931 | 11468121 |
| prenylamine | 12.14 | 0.666 | −0.303 | 0.783 | 0.221 | 0.261 | −0.799 | −0.105 | −0.116 | −0.066 | 1137095 | 11467708 |
| 8-azaguanine | 26.3 | −1.815 | 2.211 | −3.284 | −0.269 | −0.702 | −0.772 | −0.268 | 0.069 | −0.956 | 1164875 | 11467149 |
| graveoline | 14.32 | −0.675 | −0.442 | −1.868 | −1.003 | 0.781 | 0.073 | 0.025 | −0.096 | 0.255 | 1182082 | 11467822 |
| albendazole | 15.08 | −0.402 | 0.854 | −1.894 | −0.296 | −1.434 | −0.280 | 1.472 | 1.575 | 0.963 | 1185085 | 11467395 |
| peucedanin | 20 | −0.128 | 0.947 | −0.535 | 1.283 | 0.772 | 0.025 | 0.457 | 0.393 | 0.474 | 1204574 | 11488545 |
| pyrogallin | 20 | 0.735 | 1.074 | −0.276 | −1.638 | −1.159 | 0.460 | 0.111 | 0.174 | 0.005 | 1210108 | 11488369 |
| doxazosin | 8.86 | 0.032 | −0.438 | −2.021 | −0.510 | 0.525 | −0.349 | −0.173 | 0.130 | −0.738 | 1215118 | 11468006 |
| lomatin | 20 | −0.313 | −0.024 | −1.088 | −0.532 | 0.872 | 0.292 | 1.366 | 1.472 | 0.873 | 1216977 | 11488551 |
| trimethylcolchicinic acid | 11.64 | 0.624 | 0.665 | −0.160 | −0.057 | 2.869 | −0.455 | 0.463 | 0.316 | 0.667 | 1219467 | 11467728 |
| tolfenamic acid | 15.28 | −0.486 | −0.490 | −1.076 | −0.393 | 0.935 | 0.994 | −0.571 | −0.655 | −0.328 | 1258696 | 11467353 |
| tolfenamic acid | 20 | 0.781 | 1.433 | −0.820 | 0.187 | 0.019 | 0.163 | −0.743 | −0.690 | −0.701 | 1258696 | 11489262 |
| moricizine | 9.36 | 4.015 | 1.072 | −0.636 | −0.488 | −0.597 | 0.506 | −1.040 | −1.010 | −0.920 | 1267101 | 11468199 |
| noreleagnine | 20 | −0.319 | 0.384 | −0.992 | −0.406 | 2.215 | 0.363 | 0.483 | 0.459 | 0.440 | 1275107 | 11489195 |
| fenbendazole | 13.36 | −1.112 | −0.672 | −3.590 | −0.395 | −0.919 | −1.263 | −0.247 | −0.449 | 0.176 | 1281686 | 11467358 |
| ursinic acid | 20 | −0.339 | 1.207 | −1.373 | −0.377 | 1.168 | 0.385 | −0.879 | −1.088 | −0.290 | 1296906 | 11488549 |
| carteolol | 13.68 | −0.636 | 0.272 | −1.361 | −0.909 | 0.900 | 0.284 | −0.151 | −0.076 | −0.273 | 1300555 | 11467594 |
| anabasamine | 20 | 0.194 | 2.169 | −0.451 | 0.626 | 1.775 | 0.737 | −0.370 | −0.320 | −0.400 | 1307902 | 11488543 |
| 4′-methoxyflavone | 20 | −0.685 | −0.937 | −2.665 | 0.985 | −0.200 | 0.099 | 0.051 | 0.053 | 0.033 | 1308020 | 11488590 |
| etilefrine | 22.08 | −0.527 | −0.198 | −0.290 | −0.888 | 1.151 | −0.311 | 0.681 | 0.750 | 0.403 | 1326779 | 11468165 |
| gliquidone | 7.58 | 1.017 | −0.244 | −1.418 | −0.322 | 0.255 | 0.274 | 0.110 | −0.021 | 0.348 | 1327636 | 11468139 |
| dubinidine | 14.52 | −0.918 | −0.057 | −0.592 | 0.038 | −0.014 | −0.391 | −0.036 | −0.151 | 0.193 | 1336284 | 11468233 |
| dictamnine | 20 | −0.505 | −0.316 | −1.500 | 0.535 | 0.549 | 0.194 | 0.702 | 0.506 | 1.011 | 1352641 | 11488165 |
| trimetazidine | 15.02 | −0.050 | −1.226 | −0.803 | −0.534 | 0.607 | 0.229 | 0.461 | 0.474 | 0.347 | 1365793 | 11467697 |
| 7,4′-dimethoxyisoflavone | 20 | 1.005 | 0.478 | −1.267 | −0.490 | 0.773 | 0.261 | 0.197 | 0.050 | 0.413 | 1372522 | 11488001 |
| 3,7-dihydroxyflavone | 20 | −0.456 | −0.915 | −0.312 | −2.946 | −2.085 | 0.215 | 0.095 | 0.444 | −0.600 | 1386109 | 11489468 |
| levonordefrin | 21.84 | −0.087 | 1.348 | −0.710 | −1.137 | 0.294 | −0.636 | −0.224 | −0.166 | −0.293 | 1402956 | 11467887 |
| nordefrin | 20 | 0.638 | 0.219 | 0.166 | −0.895 | −0.018 | −0.951 | −0.063 | −0.318 | 0.504 | 1402956 | 11489653 |
| ethotoin | 19.58 | −0.800 | −0.485 | −1.749 | −0.745 | 1.170 | 0.248 | −0.903 | −0.691 | −1.157 | 1424515 | 11467844 |
| timolol | 12.64 | 0.053 | 0.107 | 0.175 | −0.146 | 0.440 | −0.362 | −0.595 | −0.590 | −0.481 | 1428570 | 11468096 |
| 6-benzylaminopurine | 17.76 | −0.302 | 0.316 | −0.254 | −0.549 | 1.260 | 0.518 | −0.201 | −0.258 | −0.087 | 1428839 | 11467337 |
| ondansetron | 13.64 | −0.341 | 1.494 | 0.145 | −0.331 | 0.719 | 0.375 | −0.572 | −0.443 | −0.739 | 1434439 | 11468206 |
| chlorquinaldol | 20 | −0.712 | 0.379 | −1.018 | −0.765 | 0.224 | 0.140 | −1.384 | −1.357 | −1.132 | 1449108 | 11488340 |
| azacyclonol | 14.96 | 0.047 | 0.894 | −0.594 | −0.633 | 0.492 | 0.380 | −0.448 | −0.532 | −0.247 | 1451360 | 11467241 |
| pefloxacine | 20 | −0.589 | 0.022 | −2.114 | −0.877 | −0.100 | 0.113 | −0.326 | −0.406 | −0.146 | 1461079 | 11487850 |
| 1-methylxanthine | 20 | 0.082 | 0.307 | −0.865 | −0.093 | 0.180 | 0.250 | −0.251 | −0.248 | −0.145 | 1464728 | 11489011 |
| trolox | 15.98 | −0.273 | −0.639 | −0.669 | −0.497 | −0.542 | −0.441 | −0.631 | −0.607 | −0.555 | 1470254 | 11467678 |
| N-hydroxymethylnicotinamide | 20 | 0.760 | 0.339 | 0.243 | −0.391 | 0.366 | −0.140 | 0.697 | 0.811 | 0.401 | 1492782 | 11489013 |
| 7,2′-dimethoxyflavone | 20 | −0.904 | 2.404 | −0.909 | 1.076 | 0.306 | 0.916 | −0.346 | −0.288 | −0.366 | 1499608 | 11488140 |
| molindone | 14.48 | −1.104 | −0.132 | −0.423 | −0.313 | 0.474 | −0.177 | 1.206 | 0.870 | 1.664 | 1511611 | 11468183 |
| brompheniramine | 12.52 | −0.319 | −1.424 | −1.034 | −0.479 | −0.401 | −1.195 | −0.303 | −0.163 | −0.536 | 1537011 | 11467623 |
| brompheniramine | 20 | −0.583 | 0.512 | −0.648 | 1.632 | 0.401 | −0.229 | 0.613 | −0.023 | 1.799 | 1537011 | 11489426 |
| 7-hydroxy-2′-methoxyisoflavone | 20 | −0.973 | −0.019 | −1.654 | −0.327 | 0.478 | −0.193 | 0.704 | 0.732 | 0.570 | 1539748 | 11488414 |
| foliosidine | 13.02 | −0.233 | −0.060 | −1.243 | −1.032 | 2.079 | 0.419 | −0.284 | −0.299 | −0.202 | 1540789 | 11467815 |
| 6,4′-dimethoxyflavone | 20 | −0.838 | 2.025 | −0.935 | 1.774 | 0.329 | 0.916 | −0.950 | −0.881 | −0.864 | 1552436 | 11488138 |
| diltiazem | 20 | −0.542 | −0.757 | −2.284 | −1.479 | 1.020 | 0.385 | −0.618 | −0.403 | −0.882 | 1587004 | 11489552 |
| thermopsine | 20 | −0.299 | 0.109 | −0.966 | −1.038 | −0.198 | 0.486 | −0.583 | −0.326 | −0.945 | 1592184 | 11489443 |
| lupanine | 20 | −0.176 | 1.429 | −0.156 | −0.178 | 0.560 | 0.153 | 0.340 | 0.178 | 0.637 | 1592184 | 11489505 |
| losartan | 20 | 1.208 | 0.072 | −0.833 | −0.886 | −0.340 | 0.081 | −0.345 | −0.353 | −0.223 | 1606766 | 11489493 |
| cefamandole | 20 | 0.666 | 0.897 | −0.954 | 1.278 | 1.175 | 0.222 | −0.224 | −0.374 | 0.089 | 1635952 | 11489813 |
| estradiol benzoate | 20 | −0.697 | 0.339 | −1.333 | −0.644 | 1.957 | 0.591 | −0.916 | −0.629 | −1.248 | 1661997 | 11488912 |
| sulfachloropyridazine | 14.04 | −0.675 | 0.023 | −1.776 | −0.809 | 0.928 | −0.066 | 0.119 | −0.046 | 0.438 | 1668673 | 11467863 |
| sulfachlorpyridazine | 20 | −0.951 | −0.005 | −1.163 | −0.362 | 0.611 | 0.188 | 0.033 | 0.003 | 0.038 | 1668673 | 11489758 |
| 3′,4′-dimethoxyflavone | 20 | 0.168 | −1.107 | −1.294 | 1.634 | 0.104 | −1.270 | 0.043 | −0.374 | 0.881 | 1713087 | 11488525 |
| pinocembrin | 20 | 0.098 | 0.289 | −1.277 | 0.000 | 0.845 | −1.513 | −0.178 | −0.265 | 0.076 | 1734308 | 11489486 |
| boldine | 12.22 | −1.288 | −0.637 | −1.026 | −0.529 | 0.060 | 0.505 | −0.367 | 0.105 | −1.253 | 1737132 | 11467748 |
| boldine | 20 | −0.616 | 0.728 | −1.980 | −1.070 | 0.242 | −0.754 | −0.522 | −0.828 | 0.241 | 1737132 | 11488411 |
| biochanin A, dimethyl ether | 20 | 0.667 | 0.376 | −1.779 | 0.402 | 0.251 | 0.227 | −0.814 | −0.749 | −0.753 | 1864491 | 11489519 |
| phenethyl caffeate | 20 | −1.626 | −0.517 | −2.695 | 0.387 | −2.764 | −2.848 | −1.698 | −1.604 | −1.575 | 1907763 | 11488635 |
| 4-naphthalimidobutyric acid | 20 | −0.597 | −0.403 | −0.438 | −0.619 | 1.353 | 0.276 | 0.370 | 0.199 | 0.693 | 1913604 | 11488265 |
| 4′-methoxychalcone | 20 | 0.083 | −0.085 | −0.484 | 0.178 | 0.974 | −1.182 | −0.552 | −0.423 | −0.734 | 1913676 | 11488636 |
| azathioprine | 14.42 | −0.835 | −0.254 | −1.407 | −0.321 | −0.531 | 0.343 | −1.121 | −1.320 | −0.540 | 1921128 | 11467242 |
| nifuroxazide | 14.54 | −0.395 | −0.168 | −0.624 | −0.629 | 0.535 | 0.234 | 0.138 | 0.105 | 0.186 | 1931603 | 11467703 |
| hymecromone | 20 | 0.672 | 0.191 | −0.065 | −0.521 | −0.147 | 0.214 | 0.025 | 0.103 | −0.109 | 1952709 | 11489585 |
| cefoperazone | 20 | 0.097 | 0.164 | −0.793 | −0.908 | −0.295 | 1.001 | 0.130 | 0.013 | 0.367 | 1981222 | 11489580 |
| isosafrole | 20 | −0.798 | 0.369 | −0.913 | −1.060 | 0.397 | 0.462 | −1.283 | −0.860 | −1.917 | 1984013 | 11488488 |
| 11a-acetoxykhivorin | 20 | 0.824 | −0.824 | −1.019 | −0.178 | −0.252 | 0.082 | 0.676 | 0.586 | 0.662 | 2060025 | 11488039 |
| dihydrofissinolide | 20 | 0.139 | −0.079 | −1.189 | 0.196 | 1.029 | −0.122 | −0.253 | −0.350 | 0.041 | 2060026 | 11488155 |
| 3beta-hydroxydeoxodihydrogedunin | 20 | −0.364 | 0.638 | −0.540 | 0.674 | 0.170 | 0.591 | −0.585 | −0.648 | −0.302 | 2060027 | 11488157 |
| bussein | 20 | 0.575 | −0.071 | −1.406 | 0.367 | −0.449 | −1.496 | 1.630 | 1.431 | 1.660 | 2060028 | 11488042 |
| 3beta-acetoxydeoxodihydrogedunin | 20 | 0.311 | 0.077 | −0.528 | 0.254 | 0.055 | 0.759 | −1.016 | −1.055 | −0.694 | 2060029 | 11488158 |
| carapin | 20 | 1.424 | 0.175 | −0.925 | −0.524 | 1.897 | −0.096 | 0.512 | 0.665 | 0.058 | 2060030 | 11488043 |
| cedrelone | 20 | −4.680 | −8.647 | −6.617 | −3.459 | −3.982 | −5.998 | ND | ND | ND | 2060031 | 11488044 |
| totaralolal | 20 | 0.591 | 0.829 | 1.526 | 0.460 | 1.617 | −0.226 | −0.700 | −0.401 | −1.230 | 2060034 | 11488084 |
| deacetylgedunin | 20 | −0.032 | 0.232 | −0.958 | 0.349 | −0.966 | −0.489 | −0.322 | −0.244 | −0.484 | 2060035 | 11488045 |
| ptaeroxylin | 20 | −0.025 | −0.704 | 0.160 | 0.238 | 0.259 | 0.599 | −0.853 | −0.907 | −0.641 | 2060036 | 11488099 |
| 3alpha-acetoxydihydrodeoxygedunin | 20 | 0.781 | −0.586 | −1.653 | 1.157 | 2.371 | 0.179 | −0.987 | −1.123 | −0.584 | 2060037 | 11488075 |
| deoxyandirobin | 20 | 0.894 | 1.237 | 0.232 | 0.381 | −1.077 | 1.196 | 1.352 | 1.240 | 1.246 | 2060038 | 11488048 |
| peucenin | 20 | −0.906 | 0.465 | −1.695 | −0.057 | −0.014 | 0.494 | 0.148 | 0.051 | 0.361 | 2060039 | 11488160 |
| 2-ethoxycarbonyl-2-hydroxy-5,7- | 20 | 0.511 | −1.500 | −0.260 | 0.215 | 0.774 | 0.161 | 0.280 | 0.232 | 0.273 | 2060040 | 11488031 |
| dimethoxyisoflavanone | | | | | | | | | | | | |
| griseofulvic acid | 20 | 0.176 | −1.279 | −0.469 | −0.666 | −0.323 | 0.724 | −0.370 | −0.442 | −0.209 | 2060043 | 11488028 |
| iriginol hexaaceatate | 20 | −0.291 | 0.302 | −0.565 | −0.204 | 0.815 | −0.315 | 0.928 | 0.859 | 0.938 | 2060044 | 11488216 |
| retusoquinone | 20 | −5.371 | −8.276 | −6.075 | −3.628 | −3.565 | −5.303 | ND | ND | ND | 2060045 | 11488435 |
| epoxy (4,5alpha)-4,5-dihydrosantonin | 20 | 0.133 | 0.475 | 0.041 | 0.199 | −1.367 | 0.552 | −0.736 | −0.614 | −0.901 | 2060046 | 11487977 |
| diacetyldideisovaleryl-rhodomyrtoxin | 20 | −0.779 | −0.179 | 0.875 | 1.937 | 0.967 | 0.057 | −0.776 | −0.742 | −0.703 | 2060048 | 11488606 |
| eugenitol | 20 | −0.575 | 0.166 | −1.139 | −0.541 | 1.256 | −0.041 | 0.471 | 0.168 | 0.962 | 2060049 | 11488565 |
| isoeugenitol | 20 | 0.377 | 0.995 | −0.500 | 0.369 | 1.129 | 0.662 | −0.717 | −0.647 | −0.680 | 2060051 | 11488385 |
| norstictic acid | 20 | −0.899 | 2.102 | −0.567 | 0.903 | 0.560 | 0.471 | 0.518 | 0.774 | −0.148 | 2060054 | 11489744 |
| dihydrogedunin | 20 | 0.637 | 0.315 | −0.017 | 1.079 | −0.690 | 0.460 | 0.575 | 0.613 | 0.323 | 2060055 | 11488049 |
| heteropeucenin, methyl ether | 20 | −0.034 | −0.142 | −1.476 | 0.886 | 0.256 | 0.416 | 0.299 | 0.328 | 0.217 | 2060056 | 11488161 |
| fissinolide | 20 | 0.060 | 0.175 | −1.576 | −0.324 | 0.475 | −0.167 | 0.534 | 0.385 | 0.777 | 2060057 | 11488431 |
| oleanoic acid | 20 | 0.492 | 1.083 | −0.720 | −0.910 | −0.144 | 0.754 | −1.379 | −1.408 | −1.014 | 2060058 | 11488167 |
| havanensin triacetate | 20 | 1.042 | 0.189 | −0.499 | 0.898 | 0.140 | −0.315 | 0.716 | 0.370 | 1.210 | 2060059 | 11488051 |
| deacetoxy-7-oxogedunin | 20 | 0.089 | −0.025 | −0.029 | 1.322 | −0.301 | 0.047 | −0.407 | −0.287 | −0.632 | 2060060 | 11488052 |
| dihydrospatheliachromene | 20 | −0.538 | 0.342 | −1.802 | −0.174 | 1.483 | 0.068 | −0.666 | −0.620 | −0.569 | 2060061 | 11488162 |
| 7-deacetoxy-7-oxokhivorin | 20 | 0.053 | −0.644 | −0.095 | 0.258 | 0.070 | −0.184 | 0.777 | 0.690 | 0.738 | 2060062 | 11488053 |
| khayanthone | 20 | 1.156 | 0.345 | −0.382 | 0.962 | 0.015 | −0.669 | 1.491 | 1.510 | 1.095 | 2060063 | 11488054 |
| khayasin | 20 | −0.077 | 0.871 | −0.812 | 0.007 | 0.528 | −0.281 | 0.671 | 0.798 | 0.326 | 2060064 | 11488211 |
| oxonitine | 20 | −1.338 | 0.116 | −1.730 | −1.019 | −0.167 | 0.394 | −0.636 | −0.938 | 0.147 | 2060065 | 11488170 |
| angolensic acid, methyl ester | 20 | 1.019 | −0.097 | 0.322 | 0.163 | 0.465 | −0.204 | 0.827 | 0.729 | 0.813 | 2060066 | 11488055 |
| gedunol | 20 | 0.438 | 0.637 | −1.248 | 0.897 | 0.132 | 0.758 | −1.545 | −1.561 | −1.174 | 2060067 | 11488387 |
| sarmentoside B | 20 | −0.939 | 0.526 | −2.387 | −0.760 | 0.222 | 0.251 | −1.573 | −1.615 | −1.141 | 2060068 | 11488171 |
| irigenin, 7-benzyl ether | 20 | −0.334 | −0.373 | −1.179 | 0.948 | 1.548 | −0.617 | −1.362 | −1.386 | −1.100 | 2060069 | 11488016 |
| iretol | 20 | −0.552 | −0.628 | −0.086 | −0.404 | −0.393 | 0.347 | −0.881 | −0.583 | −1.366 | 2060070 | 11488018 |
| haematommic acid, ethyl ester | 20 | −0.557 | −0.324 | −0.761 | −0.120 | 0.478 | −0.104 | 0.450 | 0.387 | 0.537 | 2060071 | 11488445 |
| rotenonic acid | 20 | 0.206 | 0.763 | −0.731 | 0.050 | 0.173 | 0.365 | −0.064 | −0.010 | −0.108 | 2060078 | 11488212 |
| dihydrogambogic acid | 20 | −5.468 | −8.298 | −6.208 | −3.431 | −2.239 | −5.191 | ND | ND | ND | 2060079 | 11488443 |
| tetrahydrogambogic acid | 20 | 1.434 | −0.668 | −1.090 | −1.438 | 0.460 | −0.932 | −0.478 | −0.409 | −0.490 | 2060080 | 11489622 |
| 2-isoprenyl-3-hydroxy-5-methyl-a-pyrone | 20 | −1.025 | 0.004 | 0.055 | −0.641 | 1.032 | −0.568 | 0.309 | 0.277 | 0.271 | 2060081 | 11489775 |
| methyl 7-desoxypurpurogallin-7-carboxylate | 20 | −0.836 | 0.028 | −2.317 | −0.254 | 0.800 | −0.229 | 0.231 | 0.287 | 0.117 | 2060082 | 11488271 |
| trimethyl ether | | | | | | | | | | | | |
| 6-hydroxyangolensic acid methyl ester | 20 | 1.224 | −0.193 | −0.490 | 0.463 | −0.249 | 0.817 | −1.426 | −1.425 | −1.206 | 2060083 | 11488057 |
| obacunol | 20 | 2.016 | 1.116 | −0.982 | 0.464 | −0.134 | 0.859 | −0.906 | −0.927 | −0.734 | 2060085 | 11488058 |
| entandrophragmin | 20 | 0.672 | 1.006 | −0.737 | 0.113 | 1.351 | −0.703 | 0.467 | 0.149 | 1.061 | 2060086 | 11488156 |
| swietenine | 20 | 1.223 | 0.027 | −0.653 | −0.241 | −0.259 | 0.244 | −1.282 | −1.274 | −1.090 | 2060087 | 11488060 |
| fraxidin methyl ether | 20 | −1.325 | 0.407 | −2.013 | −0.713 | 0.276 | −0.098 | −0.124 | −0.104 | −0.103 | 2060088 | 11488173 |
| utilin | 20 | 1.131 | 1.189 | −1.571 | −0.122 | 0.482 | −0.038 | −0.939 | −0.861 | −0.975 | 2060089 | 11488062 |
| humilin A | 20 | 0.840 | −0.106 | −1.117 | 0.314 | −0.028 | 0.423 | −0.789 | −0.820 | −0.623 | 2060090 | 11488061 |
| niloticin | 20 | 1.304 | 0.356 | −0.481 | 0.461 | 0.817 | 0.136 | 0.543 | 0.648 | 0.151 | 2060091 | 11488063 |
| 3-acetoxypregn-16-en-12,20-dione | 20 | −0.873 | 1.152 | −0.553 | −0.216 | 0.201 | −0.050 | −0.885 | −0.775 | −0.958 | 2060092 | 11488576 |
| odoratone | 20 | 0.542 | 0.474 | −1.060 | 0.367 | 0.083 | 0.707 | −0.994 | −0.988 | −0.873 | 2060093 | 11488064 |
| swietenolide-3-acetate | 20 | −0.503 | 1.431 | −0.776 | 1.006 | 0.237 | 0.668 | −1.057 | −1.093 | −0.838 | 2060094 | 11488065 |
| 1,7-dideacetoxy-1,7-dioxokhivorin | 20 | 0.042 | 0.712 | −0.142 | −0.535 | 0.352 | 0.539 | −0.558 | −0.585 | −0.355 | 2060096 | 11488178 |
| 3beta-chloroandrostanone | 20 | −0.424 | 1.376 | −1.203 | −0.860 | −0.077 | 1.021 | −1.190 | −1.192 | −0.907 | 2060098 | 11488179 |
| 7-deshydroxypyrogallin-4-carboxylic acid | 20 | 0.121 | 0.330 | −0.460 | −1.109 | −0.210 | 0.419 | −0.997 | −1.039 | −0.770 | 2060100 | 11487990 |
| 8-iodocatechin tetramethyl ether | 20 | 0.377 | 0.343 | −1.359 | 0.146 | −0.448 | 0.237 | −1.194 | −0.985 | −1.411 | 2060101 | 11488697 |
| tetramethylhaematoxylone | 20 | 0.837 | −0.771 | −0.703 | −0.921 | −0.215 | −0.056 | −0.273 | −0.365 | −0.093 | 2060102 | 11488005 |
| rotenonic acid, methyl ether | 20 | 0.582 | −4.969 | −3.898 | −3.751 | −3.513 | −3.644 | −2.268 | −1.024 | −4.305 | 2060103 | 11488941 |
| methylnorlichexanthone | 20 | −0.496 | 0.429 | −1.082 | 0.258 | 0.477 | 0.385 | −0.661 | −0.566 | −0.698 | 2060104 | 11489618 |
| irigenin trimethyl ether | 20 | −0.060 | 0.108 | −0.808 | −0.174 | −0.074 | 0.129 | −0.792 | −0.790 | −0.699 | 2060106 | 11488007 |
| 3,4-dimethoxydalbergione | 20 | −4.588 | −5.364 | −6.200 | −3.787 | −1.269 | −5.551 | −3.047 | −2.944 | −2.724 | 2060107 | 11488120 |
| 2′-methoxyformonetin | 20 | 0.477 | 1.998 | −1.138 | 0.388 | 1.137 | −1.318 | −0.484 | −0.561 | −0.296 | 2060110 | 11488004 |
| cearoin | 20 | −1.078 | −1.727 | −2.331 | −1.006 | −2.274 | −1.478 | −1.672 | −1.568 | −1.603 | 2060111 | 11489770 |
| resveratrol 4′-methyl ether | 20 | −0.225 | −0.649 | −1.056 | −0.389 | 0.225 | 0.142 | −0.005 | −0.063 | 0.065 | 2060112 | 11487862 |
| orsellinic acid | 20 | −0.320 | −1.140 | −1.311 | −0.846 | 1.236 | −0.015 | −0.348 | −0.350 | −0.333 | 2060113 | 11488121 |
| cotarnine | 20 | −0.398 | 1.285 | −0.587 | −1.103 | −0.007 | 0.068 | 0.049 | 0.159 | −0.151 | 2060114 | 11488180 |
| prenyletin | 20 | 0.919 | 0.659 | −1.166 | 0.772 | −0.090 | 0.150 | 0.113 | −0.015 | 0.282 | 2060115 | 11488066 |
| khayasin C | 20 | −1.021 | −0.324 | −1.678 | −0.095 | −0.078 | −0.654 | 0.442 | 0.033 | 1.232 | 2060116 | 11488205 |
| 13-methyl-4,4-bisnor-8,11,13-podocarpatrien- | 20 | −0.093 | −0.267 | −2.132 | −0.470 | 0.072 | 0.009 | 0.903 | 0.870 | 0.748 | 2060117 | 11488101 |
| 3-one | | | | | | | | | | | | |
| 3alpha-hydroxy-3-deoxyangolensic acid methyl | 20 | 0.949 | 0.398 | −1.268 | −0.043 | 0.799 | −0.141 | −0.456 | −0.474 | −0.377 | 2060118 | 11488071 |
| ester | | | | | | | | | | | | |
| 3-chloro-8beta-hydroxycarapin, 3,8-hemiacetal | 20 | 0.170 | 0.067 | −1.971 | −0.678 | 2.012 | 0.075 | −1.044 | −0.883 | −1.228 | 2060121 | 11488072 |
| xanthyletin | 20 | −0.674 | 0.349 | −1.741 | −0.220 | 0.217 | −0.029 | 1.029 | 0.924 | 1.085 | 2060122 | 11488181 |
| totarol-19-carboxylic acid, methyl ester | 20 | −0.739 | 2.434 | 1.019 | −0.409 | 1.290 | 0.085 | −0.323 | −0.005 | −0.853 | 2060124 | 11488182 |
| 19-hydroxytotarol | 20 | −0.207 | 0.803 | 1.198 | 0.453 | 1.558 | 1.305 | −0.041 | −0.128 | 0.086 | 2060126 | 11488085 |
| 16-deoxomexicanolide 16-methyl ether | 20 | 0.470 | −0.197 | −2.098 | −0.162 | 0.240 | −0.339 | −0.225 | −0.320 | −0.037 | 2060127 | 11488070 |
| chukrasin methyl ether | 20 | −0.822 | 0.533 | −0.818 | −1.084 | 0.777 | −0.248 | 0.352 | 0.417 | 0.198 | 2060128 | 11488183 |
| khivorin | 20 | 0.807 | −0.294 | −1.421 | −0.145 | 0.187 | 0.229 | −0.144 | −0.022 | −0.423 | 2060129 | 11488078 |
| (R)-angolensin | 20 | −0.164 | 1.320 | −0.743 | −0.444 | 0.225 | −0.063 | 0.403 | 0.703 | −0.245 | 2060130 | 11488210 |
| 2-ethoxycarbonyl-5,7-dihydroxy-8,3′,4′,5′- | 20 | −1.124 | 0.782 | −0.761 | −0.397 | 0.244 | −0.783 | −1.306 | −1.229 | −1.218 | 2060131 | 11488664 |
| tetramethoxyisoflavone | | | | | | | | | | | | |
| solidagenone | 20 | −0.409 | 0.314 | 0.084 | −0.242 | 1.734 | −0.061 | 0.127 | −0.023 | 0.441 | 2060132 | 11489574 |
| iridin | 20 | −0.441 | 0.161 | −1.958 | 0.076 | 0.927 | −0.474 | −0.755 | −0.602 | −0.990 | 2060133 | 11488014 |
| sphondin | 20 | −0.790 | 0.064 | −0.661 | 0.136 | 1.692 | 0.091 | −0.684 | −0.605 | −0.666 | 2060134 | 11489575 |
| euparin | 20 | 0.021 | −1.071 | −0.912 | −0.010 | 0.795 | 0.419 | 1.120 | 1.293 | 0.484 | 2060136 | 11488122 |
| isotectorigenin trimethyl ether | 20 | 0.976 | −0.305 | −1.465 | 0.413 | 0.359 | 0.640 | 0.437 | 0.426 | 0.418 | 2060137 | 11488394 |
| gibberellic acid | 11.54 | −0.171 | 1.388 | −0.432 | 2.622 | 0.246 | 0.714 | 0.021 | 0.066 | −0.090 | 2060138 | 11468113 |
| gibberellic acid | 20 | −0.081 | 0.413 | 0.661 | −0.439 | 0.512 | 0.031 | −0.014 | 0.077 | −0.264 | 2060138 | 11488127 |
| duartin, dimethyl ether | 20 | 1.330 | 0.049 | −0.975 | 0.556 | 0.796 | −0.531 | −1.156 | −1.362 | −0.571 | 2060139 | 11487996 |
| isobergaptene | 20 | 0.375 | −0.303 | −1.025 | −0.112 | 1.329 | 0.486 | 0.514 | 0.338 | 0.715 | 2060140 | 11488129 |
| 4,4′-dimethoxydalbergione | 20 | −0.207 | −0.089 | −2.648 | −0.993 | −0.802 | −1.432 | −0.842 | −0.677 | −0.975 | 2060141 | 11488413 |
| crassin acetate | 20 | −2.757 | 0.769 | −1.220 | 0.374 | −1.394 | −1.840 | −0.720 | −0.685 | −0.703 | 2060142 | 11488130 |
| 4-methoxy-4′-hydroxy-dalbergione | 20 | 1.548 | 1.255 | −0.877 | 0.406 | 0.639 | 0.803 | −1.401 | −1.406 | −1.071 | 2060143 | 11488378 |
| 6,3′-dimethoxyflavone | 20 | 0.413 | −2.332 | −1.534 | 1.915 | −0.882 | 0.245 | 0.072 | 0.087 | −0.021 | 2060144 | 11487847 |
| 7-deacetoxy-7-oxodeoxygedunin | 20 | 0.224 | 0.609 | −1.467 | 0.757 | −0.489 | 0.166 | −1.791 | −1.586 | −1.925 | 2060145 | 11488069 |
| 12-hydroxy-4,4-bisnor-4,8,11,13- | 20 | −0.592 | 0.589 | −0.984 | −0.484 | 0.676 | −0.516 | 0.075 | −0.281 | 0.846 | 2060146 | 11488184 |
| podocarpatetraen-3-one | | | | | | | | | | | | |
| 7-deacetylkhivorin | 20 | 0.893 | 1.348 | 0.608 | −0.049 | −0.618 | 0.079 | 0.477 | 0.543 | 0.188 | 2060147 | 11488047 |
| chrysarobin | 20 | −0.685 | 0.593 | −1.646 | −0.726 | 0.805 | −0.074 | −1.153 | −1.387 | −0.401 | 2060149 | 11488408 |
| deoxyandirobin lactone | 20 | 0.281 | 0.967 | −1.509 | −0.395 | 0.837 | 0.893 | −1.510 | −1.558 | −1.179 | 2060150 | 11488073 |
| 7beta-hydroxy-7-desacetoxykhivorinic acid, | 20 | −0.160 | 0.505 | −0.485 | −0.940 | 0.252 | −0.283 | 0.553 | 0.426 | 0.736 | 2060151 | 11488257 |
| methyl ester | | | | | | | | | | | | |
| isogedunin | 20 | 0.404 | −0.722 | −1.716 | −0.489 | 1.276 | 0.050 | 0.805 | 0.883 | 0.523 | 2060152 | 11489711 |
| 14-methoxy-4,4-bisnor-4,8,11,13- | 20 | −0.577 | −0.516 | −1.530 | −0.506 | 0.673 | −0.293 | −0.465 | −0.372 | −0.626 | 2060153 | 11488103 |
| podocarpatetraen-3-one | | | | | | | | | | | | |
| 3-deoxo-3beta-acetoxydeoxydihydrogedunin | 20 | 1.066 | −0.605 | −1.633 | 1.215 | 1.249 | 0.398 | −0.742 | −0.791 | −0.559 | 2060154 | 11488074 |
| desacetyl (7)khivorinic acid, methyl ester | 20 | 0.474 | 0.731 | −0.427 | −0.579 | 0.177 | 1.004 | −0.376 | −0.485 | −0.039 | 2060155 | 11488197 |
| prieurianin | 20 | −0.894 | 2.125 | −1.263 | 1.108 | −1.358 | −1.620 | 2.020 | 1.896 | 1.931 | 2060156 | 11488296 |
| dihydroxy (3alpha,12alpha)pregnan-20-one | 20 | −0.622 | −0.294 | −1.160 | −0.540 | 0.206 | −0.685 | −0.928 | −0.972 | −0.621 | 2060157 | 11488185 |
| deoxygedunol acetate | 20 | −0.767 | 0.032 | −1.930 | 0.137 | 1.251 | 0.095 | −0.815 | −0.850 | −0.639 | 2060158 | 11488102 |
| dihydrogedunic acid, methyl ester | 20 | 0.103 | 0.122 | −1.062 | −0.320 | 0.411 | −0.443 | 0.244 | 0.141 | 0.438 | 2060159 | 11488186 |
| deoxodeoxydihydrogedunin | 20 | 0.876 | 0.419 | −0.551 | 0.941 | 0.995 | 0.837 | −1.452 | −1.370 | −1.397 | 2060160 | 11488077 |
| obliquin | 20 | 0.539 | 1.040 | −0.268 | 0.088 | 0.977 | −1.172 | −0.313 | −0.459 | 0.105 | 2060161 | 11488164 |
| 3-deoxo-3beta-hydroxymexicanolide 16-enol | 20 | 0.290 | −0.075 | −1.922 | −0.059 | 1.279 | −0.040 | −0.203 | −0.247 | −0.133 | 2060162 | 11488080 |
| ether | | | | | | | | | | | | |
| mundoserone | 20 | −0.425 | −1.275 | −1.681 | 0.837 | −0.608 | 0.418 | −0.606 | −0.643 | −0.452 | 2060163 | 11487999 |
| dehydrodihydrorotenone | 20 | −0.172 | 0.084 | −3.034 | 0.125 | 0.051 | −0.211 | −0.001 | −0.290 | 0.535 | 2060165 | 11488000 |
| 5-hydroxyiminoisocaryophyllene | 20 | 0.181 | 0.874 | 1.325 | 0.413 | 0.922 | 0.117 | −0.023 | −0.273 | 0.421 | 2060166 | 11488136 |
| methyl 7-deshydroxypyrogallin-4-carboxylate | 20 | −0.873 | 1.034 | −1.261 | −1.695 | 0.145 | 0.133 | −1.502 | −1.394 | −1.395 | 2060167 | 11488418 |
| abienol | 20 | −0.199 | 1.677 | 0.338 | 0.334 | 2.467 | 0.211 | 0.498 | 0.091 | 1.214 | 2060168 | 11488614 |
| 2′,2′-bisepigallocatechin digallate | 20 | 0.851 | 0.573 | −0.826 | −1.132 | −0.130 | 0.616 | −0.865 | −0.836 | −0.808 | 2060169 | 11487982 |
| senecrassidiol 6-acetate | 20 | 0.328 | −0.482 | 0.557 | −0.041 | 1.016 | 0.477 | 1.420 | 1.414 | 1.085 | 2060170 | 11488132 |
| bromo-3-hydroxy-4-(succin-2-yl)-caryolane | 20 | −0.498 | −0.346 | −0.892 | −0.084 | 0.604 | 0.551 | −1.029 | −0.612 | −1.624 | 2060172 | 11489717 |
| gamma-lactone | | | | | | | | | | | | |
| cadin-4-en-10-ol | 20 | −0.101 | 0.543 | −0.041 | −0.297 | 0.180 | 0.328 | −0.610 | −0.244 | −1.252 | 2060173 | 11488477 |
| sericetin | 20 | −0.783 | −1.140 | −1.507 | 6.788 | 0.124 | −0.335 | −0.890 | −0.670 | −1.132 | 2060174 | 11489713 |
| epi(13)torulosol | 20 | 0.367 | 1.007 | 0.603 | 0.022 | 1.180 | 0.483 | 0.491 | 0.342 | 0.643 | 2060175 | 11488135 |
| 8-hydroxy-15,16-bisnor-11-labden-13-one | 20 | −1.702 | 0.871 | −0.248 | 0.798 | 0.504 | 0.550 | −0.554 | −0.078 | −1.430 | 2060176 | 11488457 |
| 1,2alpha-epoxydeacetoxydihydrogedunin | 20 | 0.074 | −0.934 | −2.000 | 0.210 | 3.350 | −0.224 | −0.921 | −1.080 | −0.468 | 2060177 | 11488081 |
| sarmentogenin | 20 | 0.694 | 1.794 | −0.191 | −0.354 | −0.151 | 0.614 | 0.508 | 0.691 | 0.077 | 2060178 | 11488208 |
| 14-methoxy-4,4-bisnor-8,11,13-podocarpatrien- | 20 | −0.060 | 2.441 | −1.068 | 1.124 | −0.109 | 1.307 | −0.872 | −0.900 | −0.612 | 2060179 | 11488219 |
| 3-one | | | | | | | | | | | | |
| dihydroptaeroxylin | 20 | 0.605 | 0.530 | −0.526 | −0.192 | 0.151 | 0.403 | −0.581 | −0.410 | −0.787 | 2060184 | 11488187 |
| homopterocarpin | 20 | 0.549 | 0.381 | −0.578 | −0.306 | −0.616 | 0.575 | −0.918 | −0.713 | −1.126 | 2060185 | 11488188 |
| strophanthidinic acid | 20 | −0.745 | 0.463 | −1.651 | −0.875 | −0.059 | 0.166 | −0.553 | −0.400 | −0.726 | 2060186 | 11488189 |
| 2-isopropyl-3-methoxycinnamic acid | 20 | 0.040 | −1.528 | −2.002 | −0.155 | 0.433 | 0.274 | 0.153 | 0.153 | 0.071 | 2060187 | 11488100 |
| hydroxy (3beta)isoallospirost-9 (11)-ene | 20 | 0.035 | −0.703 | −1.944 | −0.423 | 0.244 | −0.048 | −0.457 | −0.619 | −0.081 | 2060188 | 11488091 |
| 11-ketorockogenin acetate | 20 | −0.031 | 0.241 | −0.700 | −0.584 | 0.933 | −0.702 | −1.105 | −1.026 | −0.969 | 2060190 | 11488985 |
| 2-methylene-5-(2,5-dioxotetrahydrofuran-3-yl)- | 20 | −0.749 | −0.614 | −0.968 | −0.249 | 0.012 | 0.345 | −1.109 | −1.009 | −1.046 | 2060191 | 11489720 |
| 6-oxo-10,10-dimethylbicyclo[7:2:0]undecane | | | | | | | | | | | | |
| beta-caryophyllene alcohol | 20 | −0.960 | −0.243 | −1.859 | −0.433 | 1.180 | −1.030 | 0.155 | 0.105 | 0.264 | 2060192 | 11489543 |
| 3-amino-beta-pinene | 20 | −0.108 | 0.438 | −0.906 | −0.519 | 0.421 | 0.460 | −1.376 | −1.341 | −1.138 | 2060193 | 11489719 |
| everninic acid | 20 | −0.592 | 0.947 | −1.123 | −0.942 | 0.423 | 0.115 | 1.491 | 1.517 | 1.116 | 2060194 | 11488501 |
| 3-nor-3-oxopanasinsan-6-ol | 20 | 0.209 | 0.158 | −0.492 | −0.006 | 0.172 | 0.864 | −0.605 | −0.831 | 0.004 | 2060195 | 11489587 |
| beta-toxicarol | 20 | 0.672 | −2.000 | −1.498 | 0.899 | −0.188 | −0.239 | −1.508 | −1.773 | −0.627 | 2060196 | 11488395 |
| 2-methoxy-5 (6)epoxy-tetrahydrocaryophyllene | 20 | −1.011 | 0.358 | −0.794 | −0.699 | 0.120 | 0.933 | −0.497 | −0.466 | −0.439 | 2060197 | 11489588 |
| 12a-hydroxy-5-deoxydehydromunduserone | 20 | −0.940 | −0.089 | −1.976 | −0.179 | 0.823 | −0.179 | 0.211 | 0.193 | 0.239 | 2060198 | 11489533 |
| 3,7-epoxycaryophyllan-6-ol | 20 | −0.644 | −0.499 | −1.302 | −0.461 | 0.255 | 0.604 | −1.018 | −1.203 | −0.407 | 2060199 | 11489590 |
| 2-hydroxy-5 (6)epoxy-tetrahydrocaryophyllene | 20 | −0.772 | −0.149 | −0.836 | −0.739 | 0.074 | 0.236 | −0.347 | −0.447 | −0.043 | 2060200 | 11489591 |
| avocadyne | 20 | 0.816 | −0.151 | −0.369 | −0.266 | 0.322 | 0.738 | 0.405 | 0.457 | 0.242 | 2060201 | 11489537 |
| 3,7-epoxycaryophyllan-6-one | 20 | 0.433 | −0.022 | −0.698 | −1.003 | 0.603 | 0.131 | −0.812 | −0.824 | −0.595 | 2060202 | 11489592 |
| methylorsellinic acid, ethyl ester | 20 | 0.459 | 0.341 | −0.661 | −0.478 | −0.293 | 0.754 | −0.990 | −1.040 | −0.770 | 2060203 | 11487989 |
| clovanediol diacetate | 20 | −0.119 | −0.009 | −0.708 | −0.599 | −0.318 | 0.384 | −0.020 | 0.007 | −0.026 | 2060204 | 11489593 |
| sitosteryl acetate | 20 | −0.827 | 0.613 | −0.775 | −0.192 | 0.930 | −0.443 | −0.233 | −0.389 | 0.180 | 2060206 | 11488195 |
| 1,3-dideacetyl-7-deacetoxy-7-oxokhivorin | 20 | 0.190 | −0.006 | −1.399 | 1.329 | 1.267 | 0.226 | −0.665 | −0.716 | −0.497 | 2060207 | 11488096 |
| 3,16-dideoxymexicanolide-3beta-diol | 20 | −0.384 | −0.547 | −1.091 | 0.154 | 0.587 | −0.130 | 0.150 | 0.282 | −0.202 | 2060208 | 11488022 |
| 12a-hydroxy-9-demethylmunduserone-8- | 20 | 0.509 | 1.728 | −0.976 | −0.494 | −0.738 | 0.670 | −0.404 | −0.162 | −0.773 | 2060209 | 11488209 |
| carboxylic acid | | | | | | | | | | | | |
| 1,7-dideacetoxy-1,7-dioxo-3-deacetylkhivorin | 20 | −0.023 | −0.155 | −1.103 | −0.335 | 0.122 | 0.113 | −0.911 | −0.866 | −0.875 | 2060212 | 11488097 |
| epoxy (1,2alpha)-7-deacetoxy-7-oxo- | 20 | −0.363 | 1.049 | −0.154 | 0.419 | −0.893 | 0.935 | −1.505 | −1.598 | −0.990 | 2060213 | 11488147 |
| deoxydihydrogedunin | | | | | | | | | | | | |
| mundulone | 20 | −1.113 | −4.544 | −2.487 | 3.037 | −0.141 | −0.843 | −1.719 | −1.888 | −1.091 | 2060214 | 11488105 |
| 7-desacetoxy-6,7-dehydrogedunin | 20 | −0.973 | 2.695 | −1.891 | −1.799 | −3.492 | 0.198 | −1.430 | −0.695 | −2.631 | 2060215 | 11488277 |
| isorotenone | 20 | −1.954 | −5.273 | −4.373 | −1.329 | −0.782 | −3.397 | −3.751 | −3.017 | −4.571 | 2060216 | 11488025 |
| dihydromundulone | 20 | 0.816 | −0.792 | 0.189 | −0.530 | 0.537 | −0.210 | 0.414 | 0.513 | 0.168 | 2060217 | 11489716 |
| dihydromunduletone | 20 | 1.580 | −2.030 | −0.805 | 2.250 | −0.049 | −0.146 | −1.331 | −1.276 | −1.248 | 2060218 | 11487985 |
| caryophyllenyl acetate | 20 | −0.394 | −0.165 | −1.220 | −0.241 | 0.228 | 0.671 | 0.235 | 0.117 | 0.457 | 2060219 | 11489594 |
| 3-pinanone oxime | 20 | −0.344 | 0.209 | −0.210 | −0.415 | −0.152 | 0.014 | 0.141 | 0.031 | 0.386 | 2060220 | 11489576 |
| epiafzelechin trimethyl ether | 20 | 0.557 | 0.312 | −0.884 | −0.324 | −0.190 | 1.099 | 0.019 | −0.134 | 0.350 | 2060221 | 11489582 |
| 15-norcaryophyllen-3-one | 20 | 0.557 | 0.833 | −1.582 | 0.034 | −0.667 | 0.656 | −0.016 | −0.076 | 0.137 | 2060222 | 11489577 |
| catechin tetramethylether | 20 | 0.435 | 0.310 | −0.573 | −0.172 | −0.094 | 0.586 | −1.052 | −1.106 | −0.798 | 2060223 | 11487980 |
| epicatechin | 20 | 0.687 | 0.544 | −1.556 | −1.802 | −0.199 | 0.850 | −0.754 | −0.773 | −0.631 | 2060224 | 11487981 |
| theaflavin monogallate | 20 | 0.945 | 0.164 | −0.786 | −0.581 | −0.820 | 1.252 | −0.211 | −0.017 | −0.612 | 2060226 | 11487978 |
| xylocarpus A | 20 | 1.708 | 2.503 | −0.074 | 0.427 | 1.026 | 0.822 | −0.008 | 0.110 | −0.206 | 2060228 | 11488207 |
| epigallocatechin 3,5-digallate | 20 | 0.969 | 1.156 | −1.667 | −2.135 | 0.118 | 1.040 | −1.581 | −1.625 | −1.133 | 2060229 | 11488393 |
| 3-deacetylkhivorin | 20 | 0.398 | −0.078 | −0.317 | 0.274 | 0.619 | −0.600 | −0.661 | −0.823 | −0.272 | 2060230 | 11488046 |
| dihydrodeoxygedunin | 20 | −0.004 | 1.697 | −0.637 | 0.443 | 1.030 | 0.751 | −1.009 | −1.073 | −0.631 | 2060232 | 11488149 |
| 3,16-dideoxymexicanolide-3alpha-diol | 20 | −0.318 | 1.308 | −1.094 | −0.206 | 0.758 | 0.491 | −1.044 | −1.053 | −0.771 | 2060233 | 11488150 |
| gangaleoidin | 20 | 0.889 | −1.768 | −1.213 | −1.233 | 1.356 | −0.876 | −0.512 | −0.587 | −0.310 | 2060234 | 11488110 |
| 1 (2)alpha-epoxydeoxydihydrogedunin | 20 | 0.015 | 0.869 | −1.048 | 0.074 | 2.784 | 0.022 | −0.801 | −0.831 | −0.539 | 2060235 | 11488151 |
| deacetoxy(7)-7-oxokhivorinic acid | 20 | −0.174 | 0.363 | −1.282 | −0.539 | 2.352 | 0.816 | −0.919 | −0.937 | −0.664 | 2060237 | 11488152 |
| gyrophoric acid | 20 | −0.579 | 0.125 | −0.977 | −0.842 | 0.897 | −0.557 | −0.471 | −0.546 | −0.289 | 2060238 | 11488024 |
| merogedunin | 20 | −0.222 | 2.064 | −0.645 | −0.214 | 0.741 | 0.490 | −0.254 | −0.160 | −0.350 | 2060240 | 11488153 |
| dihydro-7-desacetyldeoxygedunin | 20 | −0.527 | 1.060 | −1.403 | −0.170 | 0.608 | 0.599 | −0.961 | −0.719 | −1.218 | 2060241 | 11488154 |
| pectolinarin | 20 | −0.100 | −0.465 | −0.475 | −0.280 | 1.058 | −0.739 | −1.381 | −1.292 | −1.349 | 2060242 | 11488026 |
| tetrahydrotrimethylhispidin | 20 | −1.602 | 0.150 | −0.667 | −0.432 | 0.719 | −0.281 | −0.448 | −0.322 | −0.649 | 2060244 | 11489774 |
| melezitose | 20 | −0.843 | 0.268 | −0.968 | −0.605 | −0.125 | 0.284 | −0.502 | 0.054 | −1.553 | 2060245 | 11488468 |
| andrographolide | 20 | −0.686 | 0.416 | −0.901 | −0.165 | −1.813 | −0.044 | −1.323 | −0.698 | −2.361 | 2060246 | 11488518 |
| 7-hydroxy-8,4′-dimethoxyisoflavone | 20 | −1.022 | 0.309 | −1.073 | −0.417 | 1.404 | 0.363 | −0.962 | −0.994 | −0.667 | 2060249 | 11489570 |
| kynuramine | 20 | 0.319 | 2.131 | −1.264 | −0.127 | 1.180 | 1.161 | 0.070 | 0.093 | 0.045 | 2060251 | 11488383 |
| kynurenine | 20 | −0.110 | 0.528 | −0.912 | −0.058 | 1.941 | 0.162 | −0.039 | −0.088 | 0.069 | 2060253 | 11489196 |
| pelletierine | 20 | −0.418 | 1.086 | −0.341 | −0.120 | −0.071 | 0.349 | 0.036 | 0.077 | −0.077 | 2060254 | 11488467 |
| triacetylresveratrol | 20 | −0.198 | −1.727 | −2.002 | −3.502 | −1.361 | 0.683 | −1.841 | −1.858 | −1.401 | 2060255 | 11489448 |
| chrysanthemyl alcohol | 20 | 0.604 | 0.964 | −0.004 | −0.734 | 0.345 | 0.264 | 0.456 | 0.495 | 0.331 | 2060256 | 11489584 |
| catechin pentaacetate | 20 | 0.652 | 1.098 | −1.126 | −2.799 | −1.649 | 0.425 | 0.269 | 0.314 | 0.161 | 2060258 | 11489583 |
| anhydrobrazilic acid | 20 | −0.072 | −1.204 | −1.007 | −0.353 | 0.388 | 0.579 | 1.730 | 1.559 | 1.678 | 2060259 | 11488012 |
| liquiritigenin | 20 | 1.353 | −0.998 | −0.429 | 1.004 | 0.301 | 0.523 | −0.749 | −0.547 | −1.069 | 2060260 | 11487857 |
| 4,7-dimethoxyflavone | 20 | −0.067 | −0.035 | −0.472 | −0.795 | 1.067 | 0.031 | 1.306 | 1.526 | 0.539 | 2060260 | 11487873 |
| zeorin | 20 | 0.495 | −0.326 | −0.387 | −0.941 | 0.166 | 0.088 | 0.381 | 0.278 | 0.571 | 2060261 | 11489643 |
| 2,3,4′-trihydroxy-4-methoxybenzophenone | 20 | −0.403 | −0.035 | −0.475 | −1.356 | −0.472 | 0.378 | −1.557 | −1.472 | −1.476 | 2060263 | 11488020 |
| dehydro (11,12)ursolic acid lactone | 20 | −0.003 | 0.599 | 0.402 | −0.429 | 0.385 | −0.175 | 0.058 | 0.104 | −0.002 | 2060264 | 11489595 |
| cholic acid, methyl ester | 20 | −0.383 | 2.202 | −1.021 | 0.065 | 0.295 | 0.018 | −0.751 | −0.852 | −0.394 | 2060265 | 11489189 |
| 11-oxoursolic acid acetate | 20 | 1.094 | −1.477 | −1.214 | −0.524 | 0.280 | −1.393 | −0.240 | −0.304 | −0.027 | 2060266 | 11489596 |
| lithocholic acid | 20 | −0.094 | −0.617 | −1.586 | −0.522 | 0.854 | 0.035 | −0.631 | −0.596 | −0.579 | 2060267 | 11489190 |
| 3-oxoursan (28-13)olide | 20 | 0.638 | 0.062 | −1.871 | −0.332 | 1.010 | 0.991 | −1.322 | −1.228 | −1.205 | 2060268 | 11489597 |
| dehydroabietamide | 20 | 0.807 | 0.588 | −0.187 | 1.642 | −0.130 | 0.030 | −0.843 | −0.731 | −0.877 | 2060270 | 11489598 |
| rhodomyrtoxin B | 20 | −1.003 | −3.678 | 0.193 | 4.272 | −3.198 | −1.949 | −1.271 | −0.783 | −1.960 | 2060271 | 11489467 |
| dihydrocaryophyllen-5-one | 20 | 0.219 | 0.364 | −1.453 | 0.090 | 0.483 | 0.620 | −0.496 | −0.513 | −0.317 | 2060272 | 11489599 |
| muurolladie-3-one | 20 | −0.720 | −0.436 | −1.161 | 0.063 | −0.490 | −0.234 | −0.747 | −0.745 | −0.569 | 2060274 | 11489600 |
| ginkgolide A | 20 | 0.380 | −0.205 | −1.019 | −0.271 | 1.848 | 0.541 | −0.247 | −0.286 | −0.123 | 2060276 | 11489194 |
| 3,8-dimethoxyflavone | 20 | 0.930 | 0.242 | −0.252 | −0.103 | 1.553 | 0.361 | 0.399 | 0.315 | 0.492 | 2060277 | 11489174 |
| oleananoic acid | 20 | −0.265 | −0.334 | −1.750 | −0.645 | 0.809 | 0.172 | −0.839 | −0.751 | −0.815 | 2060279 | 11489539 |
| neotigogenin acetate | 20 | −0.705 | −0.797 | −1.611 | −0.822 | −0.437 | −0.049 | −0.890 | −0.925 | −0.693 | 2060280 | 11488088 |
| dihydrocelastrol | 20 | −4.120 | −2.447 | −5.524 | −3.514 | −3.065 | −4.583 | −0.295 | 2.183 | −5.217 | 2060287 | 11488929 |
| chrysanthellin A | 20 | −4.075 | −2.958 | −4.661 | −1.860 | −1.565 | −4.088 | −0.126 | −0.394 | 0.474 | 2060290 | 11489451 |
| 3alpha-hydroxydeoxodihydrogedunin | 20 | 0.065 | 0.835 | −1.072 | −0.061 | 1.207 | 1.091 | −0.892 | −0.832 | −0.857 | 2060291 | 11488588 |
| tridesacetoxykhivorin | 20 | −0.863 | 1.505 | −1.476 | 0.059 | 0.504 | −0.452 | −0.649 | −0.627 | −0.515 | 2060292 | 11488174 |
| cedryl acetate | 20 | −0.311 | 0.493 | −0.176 | −0.042 | 1.566 | 0.388 | 0.067 | 0.184 | −0.149 | 2060293 | 11489728 |
| deoxysappanone B 7,3′-dimethyl ether | 20 | −1.120 | −1.027 | −2.870 | −0.637 | −1.712 | −0.951 | 2.118 | 2.250 | 1.383 | 2060294 | 11488013 |
| dihydro-obliquin | 20 | −0.141 | −0.036 | −0.661 | −0.159 | 1.045 | −0.662 | 0.233 | 0.192 | 0.303 | 2060295 | 11488166 |
| dihydrojasmonic acid | 20 | −1.223 | 0.502 | −0.149 | −0.294 | 0.827 | −0.847 | −0.313 | −0.168 | −0.505 | 2060298 | 11489466 |
| punctaporin B | 20 | 0.554 | −0.771 | −0.596 | −0.174 | 0.731 | 0.175 | −0.242 | −0.049 | −0.555 | 2060299 | 11489638 |
| isoginkgetin | 20 | 2.182 | −0.550 | −1.024 | −4.387 | 1.106 | −0.400 | −0.589 | −0.318 | −1.087 | 2060300 | 11488118 |
| diosmetin | 20 | 0.174 | −0.756 | −1.922 | −2.701 | −0.014 | −0.666 | −0.892 | −1.158 | −0.134 | 2060301 | 11489454 |
| phytol | 20 | −0.520 | −0.426 | −0.544 | −0.233 | 0.940 | 0.020 | −0.177 | −0.098 | −0.270 | 2060302 | 11489725 |
| 2-methyl-3-hydroxyethylenepyran-4-one | 20 | 0.309 | −0.367 | −0.725 | −0.608 | 0.234 | −0.244 | −0.128 | −0.012 | −0.305 | 2060303 | 11489462 |
| cellobiose (D[+]) | 20 | 0.390 | 1.049 | 0.411 | −0.571 | −0.298 | 0.380 | −0.127 | 0.051 | −0.466 | 2060304 | 11489318 |
| nonic acid | 20 | −1.043 | 0.366 | −1.857 | −0.931 | 1.211 | −0.133 | −0.080 | −0.104 | 0.061 | 2060305 | 11488911 |
| rhodinyl acetate | 20 | −0.197 | 0.959 | −0.833 | −0.631 | −0.024 | 0.137 | 0.378 | 0.774 | −0.504 | 2060306 | 11488508 |
| 3-deshydroxysappanol trimethyl ether | 20 | 0.416 | −0.268 | −0.416 | 0.033 | 1.385 | −0.948 | 0.465 | 0.557 | 0.224 | 2060307 | 11489446 |
| abscisic acid | 20 | 0.309 | 0.509 | −1.053 | −0.565 | −0.476 | 0.179 | 0.469 | 0.509 | 0.299 | 2060308 | 11489319 |
| strophanthidin | 20 | −1.016 | 0.044 | −1.573 | −0.481 | 1.036 | 0.687 | −0.789 | −0.525 | −1.094 | 2060309 | 11489070 |
| chol-11-enic acid | 20 | −0.456 | −0.496 | −1.164 | −0.652 | 0.737 | 0.383 | −0.069 | −0.195 | 0.246 | 2060311 | 11488441 |
| humulene | 20 | −0.070 | 0.403 | −1.208 | −0.232 | 0.679 | −0.100 | −0.096 | −0.188 | 0.060 | 2060313 | 11489811 |
| leoidin dimethyl ether | 20 | 0.677 | 0.448 | −0.726 | −0.320 | −0.121 | 1.032 | −1.008 | −0.595 | −1.691 | 2060314 | 11488637 |
| methyl robustone | 20 | 0.848 | −0.638 | −0.344 | 0.715 | −0.347 | 0.373 | 0.033 | 0.075 | −0.063 | 2060316 | 11488642 |
| derrusnin | 20 | 1.067 | 0.050 | −1.630 | 0.025 | 4.060 | 0.511 | 0.039 | −0.129 | 0.417 | 2060317 | 11488231 |
| antheraxanthin | 20 | −0.496 | 0.403 | −0.804 | −0.305 | 0.244 | −0.206 | 0.013 | 0.026 | −0.011 | 2060318 | 11489395 |
| 5beta-12-methoxy-4,4-bisnor-8,11,13- | 20 | −0.071 | 0.458 | −2.008 | −0.173 | 1.860 | 0.380 | −0.632 | −0.582 | −0.652 | 2060320 | 11488082 |
| podocarpatrien-3-one | | | | | | | | | | | | |
| rhoifolin | 20 | 0.089 | 0.053 | −0.683 | −0.213 | 0.471 | 0.515 | −0.838 | −0.774 | −0.768 | 2060321 | 11489457 |
| 3,7-dimethoxyflavone | 20 | −0.730 | 1.027 | −1.089 | 0.817 | 0.652 | 0.982 | −0.945 | −1.084 | −0.434 | 2060322 | 11488143 |
| 5,2′-dimethoxyflavone | 20 | −0.228 | 1.331 | −0.219 | 1.002 | 0.645 | 1.046 | −0.862 | −0.881 | −0.607 | 2060323 | 11488139 |
| carylophyllene oxide | 20 | −0.547 | 0.604 | −0.713 | −0.246 | 0.636 | 0.321 | −0.087 | 0.060 | −0.324 | 2060324 | 11488450 |
| isocorydine | 11.72 | −1.678 | −0.052 | −1.041 | −1.052 | 1.214 | 0.632 | 0.084 | 0.451 | −0.664 | 2060325 | 11467745 |
| isocorydine | 20 | 1.230 | 0.593 | −1.215 | −0.017 | −0.096 | 0.355 | −0.462 | −0.463 | −0.322 | 2060325 | 11488382 |
| bebeerine | 20 | −0.728 | 0.034 | −1.681 | −0.800 | 0.759 | 0.389 | −0.005 | −0.028 | 0.119 | 2060327 | 11489053 |
| rhodomyrtoxin | 20 | −0.636 | −2.765 | 2.413 | 2.834 | 0.397 | −1.200 | 0.357 | 0.412 | 0.206 | 2060328 | 11489445 |
| glucitol-4-gucopyanoside | 20 | −0.508 | −0.172 | −0.795 | −0.454 | 0.351 | 0.305 | −1.143 | −1.087 | −1.006 | 2060330 | 11489429 |
| caryophyllene | 20 | −0.255 | 1.810 | −0.525 | 1.045 | 0.996 | −0.121 | 0.119 | 0.216 | −0.100 | 2060331 | 11489186 |
| coniferyl alcohol | 20 | −0.771 | 0.124 | −1.094 | −0.719 | −0.450 | 0.296 | −0.593 | −0.688 | −0.251 | 2060332 | 11489430 |
| piceid | 20 | −0.341 | 1.229 | −0.826 | 0.088 | 0.862 | 0.272 | 0.110 | 0.088 | 0.124 | 2060333 | 11489184 |
| loganic acid | 20 | 1.214 | −0.276 | −0.538 | −0.193 | −0.346 | −0.485 | −0.463 | −0.274 | −0.800 | 2060334 | 11489787 |
| maackiain | 20 | −1.052 | 1.146 | −0.578 | 0.391 | 0.879 | 0.969 | −0.561 | −0.285 | −1.049 | 2060335 | 11489741 |
| 3,4-didesmethyl-5-deshydroxy-3′- | 20 | −1.198 | 0.234 | −1.279 | −1.362 | 0.189 | 0.255 | 1.215 | 1.496 | 0.371 | 2060336 | 11489773 |
| ethoxyscleroin | | | | | | | | | | | | |
| centaurein | 20 | −0.006 | −0.337 | −1.640 | −0.744 | −0.090 | −0.039 | 0.497 | 0.388 | 0.657 | 2060337 | 11489433 |
| triptophenolide | 20 | −0.599 | −2.142 | −1.067 | 1.886 | −1.072 | −0.593 | −0.110 | 0.038 | −0.350 | 2060338 | 11489434 |
| brucine | 20 | 1.605 | 0.579 | −1.270 | 0.552 | 0.930 | 0.660 | −1.106 | −1.084 | −0.887 | 2060339 | 11488380 |
| 3-benzylidenyl-levulinic acid | 20 | −0.431 | 0.543 | −0.665 | −0.170 | 0.382 | −0.165 | −0.639 | −0.553 | −0.647 | 2060340 | 11489435 |
| dihydrorobinetin | 20 | −1.070 | 0.456 | −1.259 | −3.396 | −0.393 | 0.082 | −1.192 | −1.089 | −1.124 | 2060342 | 11489459 |
| 2-propyl-3-hydroxyethylenepyran-4-one | 20 | 0.162 | 0.499 | −0.906 | −0.741 | 0.195 | −0.100 | 0.682 | 0.870 | 0.202 | 2060343 | 11489461 |
| hederacoside C | 20 | −0.672 | 0.362 | −1.226 | −0.559 | 0.079 | 0.471 | −0.809 | −0.645 | −0.952 | 2060345 | 11489439 |
| dihydrocelastryl diacetate | 20 | −4.472 | −4.966 | −4.098 | −3.309 | −3.182 | −5.617 | −1.199 | 0.241 | −3.815 | 2060346 | 11488982 |
| byssochlamic acid | 20 | −0.241 | −1.035 | −0.488 | −0.092 | 0.253 | −1.053 | −0.230 | −0.069 | −0.458 | 2060349 | 11489645 |
| 3-alpha-hydroxydeoxygedinin | 20 | 0.156 | 0.418 | −0.319 | 1.760 | 0.531 | −0.321 | −1.171 | −1.202 | −0.939 | 2060350 | 11488076 |
| 3beta-acetoxy-23-bromo-isoallospirost-9 (11)- | 20 | 0.710 | −0.670 | −2.231 | −0.260 | 1.188 | 0.565 | −0.560 | −0.585 | −0.446 | 2060351 | 11488090 |
| ene-12-one | | | | | | | | | | | | |
| catechin pentabenzoate | 20 | −1.434 | 0.169 | −1.672 | −1.326 | 0.599 | 1.272 | −0.898 | −0.815 | −0.896 | 2060352 | 11488599 |
| genistein, 8-methyl | 20 | −0.071 | 0.160 | −1.035 | −0.404 | 1.016 | 0.189 | 0.247 | 0.353 | 0.023 | 2060353 | 11489573 |
| biochanin A | 20 | 0.987 | 0.278 | −0.325 | 0.033 | 0.623 | −0.416 | −0.324 | −0.459 | −0.042 | 2060354 | 11487866 |
| bilirubin | 20 | −0.649 | 0.936 | −1.287 | −1.837 | 0.249 | 1.004 | 0.312 | 0.250 | 0.427 | 2060355 | 11488451 |
| 2,3-dihydroisogedunin | 20 | 0.804 | 0.069 | −1.287 | 0.221 | 0.780 | 0.184 | −0.471 | −0.444 | −0.449 | 2060357 | 11488563 |
| lagochilin | 20 | 0.043 | −0.029 | −0.238 | 0.277 | 0.962 | −0.862 | 0.002 | −0.036 | 0.128 | 2060358 | 11489444 |
| robustic acid | 20 | −1.213 | 0.257 | −1.831 | −1.086 | −0.119 | −0.459 | −0.930 | −0.973 | −0.587 | 2060359 | 11488981 |
| myosmine | 27.36 | −0.497 | 2.062 | −1.423 | 0.326 | 1.543 | 1.184 | 0.445 | 0.712 | −0.195 | 2060360 | 11467795 |
| beta-escin | 20 | −5.049 | −5.858 | −5.088 | −3.416 | −3.107 | −5.117 | −0.914 | −0.883 | −0.745 | 2060361 | 11488351 |
| robustic acid methyl ether | 20 | 0.497 | −0.589 | 0.474 | 0.692 | 1.431 | 0.679 | 0.230 | 0.444 | −0.223 | 2060362 | 11489617 |
| deoxykhivorin | 20 | 0.803 | −0.330 | −0.494 | 0.449 | −0.038 | 0.073 | −1.026 | −0.917 | −1.101 | 2060364 | 11488067 |
| epoxygedunin | 20 | 0.052 | 0.227 | −1.657 | 0.274 | 0.409 | 0.248 | −1.146 | −1.130 | −1.005 | 2060365 | 11488079 |
| deacetoxy-7-oxisogedunin | 20 | −0.301 | −0.566 | −0.791 | 0.332 | 0.898 | 0.153 | 0.816 | 0.821 | 0.694 | 2060366 | 11488434 |
| erysolin | 20 | −4.103 | −3.023 | −3.551 | −0.785 | −2.871 | −1.868 | −0.913 | −0.850 | −0.862 | 2060367 | 11489259 |
| abrine | 20 | 0.206 | 0.140 | −1.203 | −0.332 | 1.698 | −0.094 | 0.459 | 0.430 | 0.383 | 2060368 | 11489812 |
| ichthynone | 20 | 0.234 | −0.541 | −0.681 | −0.325 | 1.842 | 0.001 | −0.217 | −0.088 | −0.466 | 2060370 | 11488574 |
| 8beta-hydroxycarapin, 3,8-hemiacetal | 20 | 0.561 | −0.147 | 0.204 | −0.251 | 1.075 | 0.436 | 0.600 | 0.472 | 0.789 | 2060371 | 11488455 |
| carapin-8 (9)-ene | 20 | 1.357 | 0.562 | −1.272 | −0.223 | 0.766 | 0.412 | 0.121 | −0.003 | 0.284 | 2060372 | 11488068 |
| kuhlmannin | 20 | 0.333 | 1.119 | −0.487 | 0.133 | 0.443 | −0.170 | 0.624 | 0.646 | 0.408 | 2060373 | 11489808 |
| heudelottin C | 20 | −0.829 | 0.840 | −0.393 | 0.373 | 0.576 | 0.156 | −0.524 | −0.245 | −1.006 | 2060374 | 11488460 |
| diacerin | 20 | −0.692 | 0.672 | −0.608 | 0.195 | 0.228 | 0.389 | −0.446 | −0.509 | −0.239 | 2060376 | 11488639 |
| dihydro-beta-tubaic acid | 20 | −1.343 | −0.543 | −0.948 | −0.653 | 0.493 | −0.659 | −0.303 | −0.190 | −0.392 | 2060377 | 11488984 |
| 2,3-dihydroxy-6,7-dichloroquinoxaline | 20 | −0.077 | 0.984 | −1.458 | −1.689 | 0.085 | 0.279 | −0.724 | −0.712 | −0.565 | 2060378 | 11488353 |
| retusin dimethyl ether | 20 | 0.317 | 0.241 | −1.263 | 0.378 | 0.069 | −0.199 | −0.135 | −0.033 | −0.331 | 2060379 | 11488631 |
| betamethasone | 20 | −0.984 | 0.559 | −1.063 | 0.849 | 0.541 | 0.130 | 0.740 | 0.633 | 0.853 | 2060380 | 11488222 |
| epoxy (1,11)humulene | 20 | 1.157 | 0.320 | −0.802 | −0.509 | −0.355 | 0.979 | −1.172 | −1.115 | −1.021 | 2060382 | 11489579 |
| deltaline | 20 | −0.735 | 0.619 | −1.231 | −0.496 | 1.654 | 0.271 | −0.220 | −0.062 | −0.422 | 2060386 | 11488933 |
| 3beta,7beta- | 20 | −0.292 | −0.198 | −0.810 | −0.208 | 1.848 | 0.161 | −1.124 | −1.228 | −0.653 | 2060390 | 11488191 |
| diacetoxydeoxodeacetoxydeoxydihydrogedunin | | | | | | | | | | | | |
| 2,3,4-trihydroxy-4′-ethoxybenzophenone | 20 | −0.413 | 1.562 | −2.368 | −1.185 | 0.052 | 0.440 | −0.823 | −0.896 | −0.478 | 2060392 | 11488240 |
| 2′,4′-dihydroxychalcone 4′-glucoside | 20 | 0.556 | 0.137 | −1.358 | −0.532 | 1.144 | −0.225 | −0.189 | −0.247 | 0.004 | 2060396 | 11488258 |
| sappanone A dimethyl ether | 20 | −1.045 | −0.310 | −1.865 | 0.017 | −2.429 | −0.664 | 0.450 | 0.495 | 0.248 | 2060397 | 11488628 |
| cholestan-3beta,5alpha,6beta-triol | 20 | −0.869 | 0.747 | 0.120 | −0.575 | 1.000 | 0.815 | 0.382 | 0.523 | 0.074 | 2060399 | 11488442 |
| 18-aminoabieta-8,11,13-triene sulfate | 20 | −0.143 | 0.999 | −0.762 | −0.519 | 2.176 | 0.507 | −0.727 | −0.771 | −0.462 | 2060400 | 11488230 |
| 5alpha-12-methoxy-4,4-bisnor-8,11,13- | 20 | −1.914 | −0.133 | −1.993 | −0.939 | 0.625 | −0.239 | 0.129 | 0.140 | 0.061 | 2060402 | 11488570 |
| podocarpatrien-3-one | | | | | | | | | | | | |
| mucronulatol | 20 | 1.396 | −0.648 | −0.847 | 0.006 | −0.187 | −0.365 | −0.345 | −0.104 | −0.729 | 2060403 | 11489650 |
| 8-hydroxycarapinic acid | 20 | 0.410 | 1.376 | −0.475 | 1.077 | 0.309 | 0.767 | −1.182 | −1.218 | −0.844 | 2060405 | 11488218 |
| coumarinic acid methyl ether | 20 | −0.844 | 0.822 | −2.099 | −1.070 | 0.917 | 0.170 | 0.143 | 0.031 | 0.383 | 2060406 | 11488269 |
| dimethylcaffeic acid | 20 | 0.147 | 1.552 | −1.206 | −0.061 | −0.142 | 0.804 | −0.924 | −0.838 | −0.885 | 2060407 | 11488228 |
| 3beta-acetoxydeoxyangolensic acid, methyl | 20 | −0.316 | 0.081 | −0.881 | −0.388 | −0.319 | −0.069 | 0.772 | 0.446 | 1.332 | 2060408 | 11488201 |
| ester | | | | | | | | | | | | |
| 1-deacetoxy-1-oxo-3,7-dideacetylkhivorin | 20 | −0.413 | −0.222 | −1.776 | −0.321 | 0.626 | −0.411 | −0.525 | −0.704 | −0.020 | 2060410 | 11488259 |
| fisetinidol | 20 | −1.412 | 0.542 | −1.804 | −1.528 | −0.215 | 0.234 | −0.998 | −1.107 | −0.536 | 2060411 | 11488239 |
| cholestanone | 20 | −0.208 | 1.206 | −2.192 | −1.065 | 1.864 | 0.272 | 0.165 | 0.147 | 0.214 | 2060413 | 11488432 |
| 6,7-dichloro-3-hydroxy-2-quinoxalinecarboxylic | 20 | 0.109 | 0.893 | −0.131 | −0.521 | 0.666 | 0.217 | −0.320 | −0.313 | −0.230 | 2060414 | 11488313 |
| acid | | | | | | | | | | | | |
| 2-mercaptobenzothiazole | 20 | −0.830 | −0.408 | −0.672 | −0.486 | 0.354 | −0.026 | −0.211 | −0.231 | −0.097 | 2060416 | 11489482 |
| tubaic acid | 20 | −0.569 | 0.416 | 0.150 | −0.489 | 0.646 | 0.089 | −0.746 | −1.065 | 0.070 | 2060417 | 11489734 |
| 8,2′-dimethoxyflavone | 20 | −1.096 | 0.456 | −0.556 | 1.282 | 1.219 | 0.864 | −0.593 | −0.414 | −0.811 | 2060418 | 11488142 |
| 3beta-hydroxydeoxodihydrodeoxygedunin | 20 | −1.397 | 0.202 | −0.656 | 1.048 | 1.488 | 0.142 | −0.747 | −0.849 | −0.359 | 2060420 | 11488256 |
| larixol | 20 | 0.085 | 0.051 | −0.221 | −0.141 | 1.068 | 0.944 | −0.507 | −0.466 | −0.552 | 2060421 | 11488133 |
| 2,4-dihydroxy-3,4-dimethoxy-4′- | 20 | −0.048 | 0.418 | −1.130 | 0.089 | −0.264 | 0.559 | −1.530 | −1.393 | −1.568 | 2060422 | 11487979 |
| ethoxybenzophenone | | | | | | | | | | | | |
| 3alpha-hydroxy-4,4-bisnor-8,11,13- | 20 | 0.056 | −0.736 | −0.574 | −0.339 | 0.477 | 0.790 | −0.250 | −0.023 | −0.717 | 2060424 | 11488117 |
| podocarpatriene | | | | | | | | | | | | |
| dihydrogeduninic acid, methyl ester | 20 | 0.488 | 0.519 | −1.004 | −0.571 | 0.820 | 0.117 | 0.739 | 0.881 | 0.289 | 2060425 | 11488577 |
| 2-methoxyresorcinol | 20 | 0.514 | 0.150 | 0.266 | −0.536 | 0.864 | −0.146 | 0.218 | −0.150 | 0.954 | 2060426 | 11489652 |
| 2-ethoxycarbonyl-2- | 20 | −0.703 | 0.391 | −1.268 | −0.404 | 0.483 | 0.521 | 0.003 | 0.155 | −0.356 | 2060428 | 11489748 |
| ethoxyoxaloyloxydihydrochrysin dimethyl ether | | | | | | | | | | | | |
| cymarin | 20 | −1.155 | −0.332 | −1.849 | −0.996 | 0.695 | 0.356 | −0.473 | −0.412 | −0.464 | 2060429 | 11488249 |
| 10-hydroxycamtothecin | 20 | −1.466 | 0.468 | −2.588 | 0.725 | −1.681 | −2.397 | −1.137 | −1.180 | −0.782 | 2060432 | 11489476 |
| buddleoflavonoloside | 20 | 0.169 | 0.643 | 0.006 | 0.050 | 0.265 | 0.736 | 0.073 | 0.114 | 0.017 | 2060433 | 11488447 |
| 6-acetoxyangolensic acid methyl ester | 20 | 0.671 | −0.780 | −0.663 | −0.055 | 1.137 | −0.105 | 1.379 | 1.291 | 1.272 | 2060435 | 11488566 |
| chaulmosulfone | 20 | −0.010 | −0.078 | −0.289 | −0.993 | 0.935 | −0.036 | 0.157 | 0.083 | 0.305 | 2060436 | 11489735 |
| coenzyme Q10 | 20 | 0.166 | 0.525 | −1.758 | 0.920 | 1.179 | 0.636 | 0.366 | 0.391 | 0.226 | 2060439 | 11488542 |
| emodic acid | 20 | 0.262 | −0.639 | −0.976 | −0.966 | 1.403 | 0.505 | −1.336 | −1.210 | −1.286 | 2060441 | 11488237 |
| ethylnorepinephrine | 20 | −0.508 | 0.711 | −1.017 | −2.053 | −0.396 | −0.439 | −0.475 | −0.195 | −0.921 | 2060442 | 11489460 |
| theaflavanin | 20 | −1.349 | 1.006 | −0.665 | −2.074 | −0.040 | −0.166 | −0.695 | −0.764 | −0.333 | 2060444 | 11488983 |
| 3beta-hydroxydeoxydesacetoxy-7-oxogedunin | 20 | 0.688 | 0.198 | −0.615 | −0.064 | 0.662 | 0.306 | −0.864 | −1.027 | −0.319 | 2060446 | 11488190 |
| tetrahydrosappanone A | 20 | −0.222 | −0.371 | 0.642 | 0.053 | 0.274 | −0.187 | −0.421 | −0.737 | 0.337 | 2060448 | 11489736 |
| crustecdysone | 20 | −0.458 | 0.693 | −0.545 | −0.721 | 0.657 | 0.468 | 0.354 | 0.487 | 0.062 | 2060451 | 11488288 |
| quinamide | 20 | −0.397 | 1.161 | −1.034 | −0.518 | 0.405 | 0.359 | −0.139 | −0.529 | 0.735 | 2060452 | 11488238 |
| tetrachloroisophthalonitrile | 20 | −5.297 | −8.366 | −6.675 | −4.269 | −3.975 | −5.292 | −3.510 | −3.990 | −1.780 | 2060453 | 11489465 |
| hieracin | 20 | 1.425 | 0.806 | −0.557 | −1.902 | −0.329 | 0.057 | −0.648 | −0.421 | −1.009 | 2060454 | 11488557 |
| trimedlure | 20 | −0.267 | 1.437 | −0.802 | −0.630 | 0.458 | 0.122 | −0.323 | −0.291 | −0.280 | 2060456 | 11488352 |
| genkwanin | 20 | −0.323 | 0.957 | −2.090 | −0.491 | 3.337 | −0.037 | 0.425 | 0.407 | 0.389 | 2060457 | 11489171 |
| dipyrocetyl | 20 | 0.452 | 1.278 | −1.355 | −0.363 | 1.260 | 0.865 | −0.311 | −0.242 | −0.341 | 2060458 | 11488233 |
| ancitabine | 20 | −0.655 | −0.629 | −2.731 | −0.986 | −0.189 | −0.158 | −1.115 | −1.182 | −0.726 | 2060459 | 11489470 |
| isoduartin methyl ether | 20 | −0.603 | 0.032 | −2.055 | −0.777 | 0.599 | 0.882 | −0.182 | −0.008 | −0.434 | 2060461 | 11488909 |
| isotectorigenin, 7-methyl ether | 20 | 0.633 | −1.101 | −1.606 | 0.574 | 0.644 | −0.477 | −0.592 | −0.674 | −0.360 | 2060463 | 11488033 |
| tetrac | 20 | 1.361 | 1.510 | −0.677 | −1.457 | 2.718 | 0.674 | 0.811 | 0.714 | 0.898 | 2060466 | 11488361 |
| sappanone A trimethyl ether | 20 | −0.259 | −0.015 | −0.980 | −0.290 | 0.120 | −0.471 | 1.090 | 1.458 | 0.094 | 2060467 | 11489793 |
| menthyl benzoate | 20 | −0.151 | 0.906 | −1.026 | 0.016 | 1.375 | −0.724 | −0.115 | 0.047 | −0.336 | 2060468 | 11488966 |
| 6alpha-methylprednisolone acetate | 20 | −0.480 | 0.169 | −1.192 | −0.462 | 0.321 | −0.364 | −0.130 | −0.518 | 0.750 | 2060469 | 11488343 |
| austricine | 20 | −0.394 | 0.158 | −1.268 | −0.711 | 0.665 | 0.842 | 0.165 | 0.081 | 0.349 | 2060471 | 11488292 |
| canrenoic acid | 20 | −0.004 | −0.434 | −0.973 | −0.749 | 0.803 | 0.732 | 0.072 | 0.154 | −0.047 | 2060472 | 11488887 |
| alpinetin methyl ether | 20 | 0.220 | 0.801 | −0.694 | −0.286 | 0.827 | −0.145 | −0.565 | −0.437 | −0.717 | 2060475 | 11489173 |
| chrysin dimethyl ether | 20 | −0.006 | −0.343 | −1.043 | 0.541 | 1.158 | −1.016 | 0.480 | 0.444 | 0.470 | 2060475 | 11489175 |
| leucomisine | 16.24 | −0.439 | −0.558 | −1.034 | 0.151 | 0.368 | −0.568 | −0.450 | −0.405 | −0.470 | 2060476 | 11468232 |
| leucodin | 20 | −0.525 | −1.027 | −1.134 | −0.355 | 0.695 | −0.301 | −0.300 | −0.270 | −0.261 | 2060476 | 11489473 |
| mepartricin | 20 | −0.568 | −0.392 | −1.431 | −0.309 | −0.251 | 0.264 | −0.710 | −0.728 | −0.464 | 2060478 | 11488950 |
| N-acetylaspartic acid | 20 | −0.266 | 0.521 | −0.456 | −0.663 | 0.309 | 0.753 | −0.670 | −0.436 | −1.046 | 2060483 | 11488608 |
| acetylsalicylsalicylic acid | 13.32 | −0.840 | 1.663 | −1.621 | −0.808 | 0.399 | −0.200 | −0.301 | −0.249 | −0.389 | 2060486 | 11467246 |
| diplosalsalate | 20 | −0.204 | −0.137 | −0.860 | 0.008 | 0.306 | 0.787 | −1.001 | −0.994 | −0.766 | 2060486 | 11489480 |
| (+)-linalool | 20 | −0.653 | −0.323 | −1.338 | −0.810 | 0.481 | 0.690 | −0.007 | 0.131 | −0.342 | 2060488 | 11489760 |
| selinidin | 20 | 0.773 | −0.419 | −0.359 | 0.013 | 0.838 | −0.478 | −0.347 | −0.328 | −0.285 | 2060489 | 11488316 |
| pteryxin | 20 | −0.681 | −0.082 | −1.515 | 0.300 | 1.106 | 0.711 | −0.101 | −0.097 | −0.108 | 2060490 | 11488561 |
| dihydrosamidin | 20 | 0.476 | 0.085 | −0.826 | 0.199 | 4.794 | 0.147 | 1.854 | 1.843 | 1.477 | 2060491 | 11488556 |
| deoxysappanone B trimethyl ether | 20 | −0.068 | 1.619 | −1.507 | −0.194 | 1.064 | 0.336 | 0.993 | 1.050 | 0.613 | 2060494 | 11488003 |
| 2′,2′-bisepigallocatechin monogallate | 20 | 0.442 | 0.545 | −1.318 | −2.350 | 0.428 | 0.246 | −0.573 | −0.545 | −0.575 | 2060495 | 11487993 |
| linamarin | 20 | 0.796 | 0.025 | −1.611 | −0.333 | −0.189 | −0.052 | −1.024 | −0.847 | −1.232 | 2060497 | 11489788 |
| apiin | 20 | −0.631 | −0.267 | −1.444 | 0.029 | 0.322 | −0.697 | −0.032 | −0.150 | 0.250 | 2060499 | 11489616 |
| felamidin | 20 | 0.224 | 0.238 | −0.475 | 0.600 | 0.710 | 0.921 | 0.966 | 0.835 | 1.050 | 2060501 | 11488547 |
| acetosyringone | 20 | −0.432 | 0.040 | −1.266 | −0.370 | 0.269 | 0.310 | 0.029 | −0.040 | 0.201 | 2060502 | 11488245 |
| (−)-asarinin | 20 | −0.632 | 0.276 | −0.505 | −0.439 | −0.229 | 0.607 | −1.167 | −0.726 | −1.858 | 2060503 | 11488627 |
| 3-methylorsellinic acid | 20 | −0.494 | 1.932 | −1.253 | −0.030 | 0.860 | 0.321 | −1.005 | −1.318 | −0.108 | 2060504 | 11488229 |
| dihydrolonchocarpenin | 20 | −0.698 | −0.259 | −0.977 | 0.277 | 0.860 | 0.022 | −0.672 | −0.562 | −0.680 | 2069224 | 11488980 |
| theaflavin digallate | 20 | −1.408 | 1.490 | −0.518 | −0.326 | 0.718 | 0.601 | 0.783 | 0.948 | 0.275 | 2069225 | 11488461 |
| dehydrovariabilin | 20 | 0.309 | −0.517 | −1.225 | 0.951 | 0.167 | 0.043 | 0.367 | 0.277 | 0.416 | 2069226 | 11487874 |
| 2-methoxyxanthone | 20 | −0.428 | −0.087 | −1.044 | 0.540 | 1.398 | 1.007 | −0.467 | −0.710 | 0.165 | 2069228 | 11488241 |
| 2-hydroxyxanthone | 20 | −0.402 | −0.916 | −1.863 | −0.245 | 1.660 | 0.238 | −0.364 | −0.173 | −0.652 | 2069229 | 11489538 |
| chlorpheniramine | 20 | 0.292 | −0.163 | −0.875 | −0.443 | 1.228 | 0.766 | 0.089 | 0.043 | 0.103 | 2069230 | 11487972 |
| 3-prenyl-4-hydroxyacetophenone | 20 | −0.696 | 1.387 | −1.066 | −0.010 | 2.418 | 0.220 | 0.607 | 0.477 | 0.804 | 2069231 | 11488405 |
| estriol methyl ether | 20 | 0.054 | −0.115 | −1.611 | −0.046 | 0.299 | 0.241 | −0.432 | −0.309 | −0.643 | 2069233 | 11487827 |
| (+)-bicuculline | 20 | −0.572 | 0.790 | −0.812 | −0.945 | 0.428 | −0.116 | −0.189 | −0.028 | −0.495 | 2069234 | 11488533 |
| 1,4,5,8-tetrahydroxy-2,6- | 20 | −0.853 | −0.346 | −0.344 | −0.662 | 0.772 | 0.005 | −1.123 | −1.178 | −0.739 | 2069239 | 11489566 |
| dimethylanthroquinone | | | | | | | | | | | | |
| tetrahydrocortisone-3,21-diacetate | 20 | −0.030 | 0.067 | −0.664 | 0.074 | 0.456 | −0.397 | −0.320 | −0.558 | 0.269 | 2069240 | 11489642 |
| estradiol methyl ether | 20 | 0.510 | 0.823 | −0.685 | −0.007 | 0.411 | 1.670 | 0.099 | 0.214 | −0.209 | 2069241 | 11487828 |
| 3,5-diprenyl-4-hydroxyacetophenone | 20 | −0.293 | −0.362 | −0.582 | −0.022 | 1.014 | −0.220 | 0.149 | −0.036 | 0.538 | 2069242 | 11488425 |
| 2′,beta-dihydroxychalcone | 20 | −0.801 | −0.920 | −1.791 | −0.044 | 0.194 | −0.260 | 0.097 | −0.188 | 0.600 | 2069243 | 11488032 |
| norstictic acid | 20 | 0.730 | 0.430 | −0.613 | −0.628 | −0.426 | 0.467 | −0.800 | −0.639 | −1.023 | 2069246 | 11487987 |
| retusin 7-methyl ether | 20 | −0.245 | −0.148 | −0.627 | −0.603 | 0.088 | 0.412 | 0.788 | 0.765 | 0.631 | 2069249 | 11487871 |
| avocadyne | 20 | −0.546 | −0.278 | −0.646 | −0.029 | 0.611 | −0.049 | −1.062 | −1.019 | −0.897 | 2069250 | 11488344 |
| 1,3-dideacetylkhivorin | 20 | 0.507 | 0.959 | −0.622 | 0.051 | 0.963 | 0.887 | −0.693 | −0.349 | −1.275 | 2069251 | 11488567 |
| prieuranin acetate | 20 | 1.518 | 1.574 | −0.879 | −0.074 | −0.400 | 0.615 | −1.113 | −0.904 | −1.381 | 2069252 | 11488059 |
| bussein | 20 | −0.052 | 0.214 | −0.599 | −0.807 | 0.656 | 0.622 | −0.809 | −0.683 | −0.868 | 2069253 | 11488438 |
| lobaric acid | 20 | 0.082 | −0.552 | −0.427 | −0.164 | 0.860 | −0.020 | −0.455 | −0.499 | −0.343 | 2069254 | 11488126 |
| irigenol | 20 | 0.249 | 0.611 | −0.810 | −0.374 | −1.001 | 0.410 | −1.274 | −0.838 | −1.938 | 2069255 | 11488617 |
| 6-methoxyprosogerin B diethyl ether | 20 | −0.627 | −0.465 | −1.860 | 0.708 | −1.331 | 0.479 | 1.074 | 0.750 | 1.499 | 2069256 | 11488571 |
| isokobusone | 20 | −0.090 | −0.166 | −0.507 | −0.283 | −0.201 | 0.480 | −0.848 | −0.813 | −0.713 | 2069257 | 11489589 |
| koparin 2′-methyl ether | 20 | 0.024 | −0.071 | −0.532 | −0.442 | 0.126 | 0.007 | −1.151 | −1.461 | −0.309 | 2069258 | 11488629 |
| epiandrostanediol | 20 | 1.143 | 1.132 | −0.207 | −0.156 | 0.113 | −0.201 | −0.069 | −0.203 | 0.190 | 2069259 | 11488625 |
| rutilantinone | 20 | 0.192 | −2.936 | −2.979 | −3.758 | 2.445 | 0.372 | −1.917 | −1.152 | −3.039 | 2069260 | 11488268 |
| alpha-toxicarol | 20 | 0.304 | −2.946 | −1.982 | 0.820 | 0.101 | −2.637 | −0.046 | 0.124 | −0.345 | 2069261 | 11489649 |
| 3,4,5-trimethoxycinnamaldehyde | 20 | 1.061 | 0.145 | 1.043 | 0.647 | −0.228 | −0.808 | −0.523 | −0.724 | 0.025 | 2069262 | 11489656 |
| 5alpha-androstan-3beta,17beta-diol | 20 | −0.204 | 0.359 | −0.454 | −1.116 | 0.607 | 0.280 | 0.123 | 0.059 | 0.270 | 2069264 | 11488427 |
| 5alpha-androstan-3,17-dione | 20 | −0.579 | −0.192 | −0.601 | 0.107 | 0.649 | 0.048 | −0.499 | −0.426 | −0.509 | 2069265 | 11488196 |
| ephedrine | 20 | −0.536 | −1.049 | −2.342 | −0.834 | 1.633 | 0.077 | −0.074 | −0.100 | −0.056 | 2069266 | 11487953 |
| praesterone acetate | 20 | 1.018 | 0.585 | 1.084 | 1.329 | 0.054 | −0.521 | −0.532 | −0.477 | −0.457 | 2069268 | 11488946 |
| allodeoxycholic acid | 20 | −1.161 | −1.212 | −2.126 | 0.404 | −0.164 | −2.903 | −0.905 | −0.903 | −0.773 | 2069269 | 11489768 |
| 5alpha-cholestan-3beta-ol-6-one | 20 | −0.430 | 2.248 | 0.811 | 0.479 | 0.397 | 1.006 | −0.731 | −0.603 | −0.862 | 2069270 | 11488620 |
| 1S,2R-phenylpropanolamine | 20 | 0.296 | −0.204 | −1.507 | −0.137 | 0.721 | 0.280 | −0.539 | −0.484 | −0.598 | 2069271 | 11487832 |
| lycopodine | 20 | −0.466 | −0.283 | −1.139 | −0.239 | 0.500 | −0.093 | −0.150 | −0.028 | −0.411 | 2069272 | 11489810 |
| chondrosine | 20 | −0.800 | 0.150 | −1.759 | −1.001 | 0.741 | 0.040 | −0.743 | −0.782 | −0.476 | 2069273 | 11488422 |
| haematoporphyrin | 20 | −1.933 | −0.917 | −3.312 | −3.662 | 1.666 | 0.324 | 0.309 | 0.251 | 0.416 | 2069274 | 11488252 |
| glycyrrhizic acid | 20 | 0.254 | 1.266 | −0.746 | −0.606 | 0.578 | 0.339 | −0.424 | −0.227 | −0.753 | 2069275 | 11489197 |
| irigenin, dibenzyl ether | 20 | −1.724 | 0.867 | −0.793 | 0.178 | 0.088 | 0.012 | −0.325 | −0.163 | −0.602 | 2069276 | 11488490 |
| haematommic acid | 20 | −0.412 | 2.409 | −0.740 | 0.002 | −1.077 | 0.631 | −0.410 | −0.078 | −1.067 | 2069278 | 11489738 |
| N-methylisoleucine | 20 | 1.026 | −0.463 | 0.372 | −0.210 | 0.300 | −0.451 | 0.772 | 0.676 | 0.850 | 2069279 | 11489655 |
| isopeonol | 20 | −1.180 | 2.649 | −0.831 | 0.271 | −0.219 | 0.734 | −0.109 | 0.134 | −0.635 | 2069280 | 11489740 |
| estrone benzoate | 20 | 0.154 | 0.592 | −0.807 | −0.277 | 0.210 | 0.406 | 0.061 | 0.190 | −0.179 | 2069281 | 11489603 |
| naproxol | 20 | 0.249 | 0.350 | −1.522 | −0.500 | −0.319 | 0.993 | −0.895 | −1.214 | −0.018 | 2069284 | 11488310 |
| arthonioic acid | 20 | −0.413 | −0.458 | −2.597 | −0.493 | 1.167 | −0.019 | −0.668 | −0.590 | −0.657 | 2069285 | 11489540 |
| ergosta-7,22-dien-3-one | 20 | −0.892 | 0.167 | −2.474 | −0.544 | 0.912 | 0.221 | −0.708 | −0.764 | −0.415 | 2069286 | 11489559 |
| dihydrojasmonic acid, methyl ester | 20 | 0.971 | −0.049 | −0.452 | −0.204 | −0.386 | −0.281 | 0.948 | 0.831 | 1.068 | 2069287 | 11488374 |
| 2,3-diacetoxy-7,8-epoxy-24,29-dinor-1,3,5- | 20 | 0.270 | 1.817 | −0.165 | −1.396 | −0.501 | −0.661 | −0.993 | −0.774 | −1.269 | 2069289 | 11488529 |
| friedelatriene-20-carboxylic acid | | | | | | | | | | | | |
| picropodophyllotoxin | 20 | −0.107 | −0.952 | −2.402 | −0.349 | −1.202 | −1.445 | 1.512 | 1.581 | 1.129 | 2069291 | 11488336 |
| bisanhydrorutilantinone | 20 | −0.493 | 0.217 | −1.755 | −2.223 | 0.236 | 0.148 | −0.727 | −0.268 | −1.532 | 2069293 | 11488469 |
| candesartan cilextil | 20 | 1.842 | 0.721 | −1.116 | −0.743 | 1.855 | 1.328 | −0.432 | −0.259 | −0.658 | 2069295 | 11488301 |
| cholesteryl acetate | 20 | −1.471 | 0.018 | −2.138 | −0.657 | 0.524 | 0.097 | 0.228 | 0.273 | 0.142 | 2069297 | 11489613 |
| acetriazoic acid | 20 | 0.205 | −0.024 | −0.938 | −0.059 | 1.158 | −1.416 | −0.710 | −0.870 | −0.320 | 2069298 | 11487844 |
| methyl 3beta,12-dihydroxy-11- | 20 | −0.315 | −0.092 | −1.387 | −0.760 | 0.918 | −0.005 | −0.063 | 0.054 | −0.217 | 2069303 | 11489068 |
| ketoisoallospirostan-3-hemisuccinate | | | | | | | | | | | | |
| androsta-1,4-dien-3,17-dione | 20 | 0.162 | −0.063 | −0.500 | 0.152 | 0.068 | 0.261 | −0.180 | −0.143 | −0.189 | 2069306 | 11489639 |
| derrubone | 20 | −0.568 | −3.786 | −0.499 | −1.272 | 1.419 | −0.126 | −0.409 | −0.626 | 0.066 | 2069308 | 11487864 |
| beta-dihydrorotenone | 20 | −0.184 | −2.497 | −2.771 | 1.079 | 0.122 | −0.408 | −0.818 | −0.828 | −0.695 | 2069309 | 11488002 |
| 5,7-dimethoxyisoflavone | 20 | −0.298 | −0.194 | −1.335 | −0.235 | 0.573 | 0.046 | 0.945 | 0.916 | 0.773 | 2069315 | 11487861 |
| tyrphostin B44 | 20 | −0.628 | −0.184 | −0.858 | −0.075 | −0.132 | −1.355 | −0.751 | −0.794 | −0.481 | 2069318 | 11489626 |
| sinapic acid methyl ether | 20 | −0.934 | 0.600 | −0.736 | −0.716 | 0.722 | 0.207 | −1.103 | −1.058 | −1.015 | 2069324 | 11489771 |
| juarezic acid | 20 | −0.106 | 0.624 | −0.449 | −0.821 | 0.345 | −0.113 | −0.147 | 0.108 | −0.650 | 2069325 | 11488633 |
| obtusaquinone | 20 | −4.658 | −8.654 | −6.409 | −3.829 | −1.119 | −5.381 | ND | ND | ND | 2069327 | 11488111 |
| ginkgetin | 20 | −1.523 | −2.623 | −3.855 | −4.324 | −1.347 | −3.027 | −2.228 | −2.585 | −1.101 | 2069329 | 11487870 |
| flavokawain B | 20 | 0.583 | 0.641 | −0.298 | −0.302 | 0.292 | 0.708 | −1.453 | −1.423 | −1.291 | 2069330 | 11487992 |
| stigmasta-4,22-dien-3-one | 20 | 0.023 | 0.796 | −1.294 | −0.299 | 0.550 | 0.434 | −0.138 | −0.094 | −0.118 | 2069333 | 11488954 |
| suprofen methyl ester | 20 | 0.611 | 0.771 | −1.160 | 0.577 | 0.591 | −0.191 | 0.139 | 0.216 | −0.048 | 2069337 | 11489182 |
| epicatechin | 20 | −1.380 | 0.949 | −1.915 | −2.452 | −0.465 | 0.110 | 0.431 | 0.310 | 0.641 | 2069338 | 11488281 |
| 2-benzoyl-5-methoxybenzoquinone | 20 | −0.284 | 0.353 | −1.489 | −0.229 | −0.542 | 0.206 | −0.699 | −0.511 | −0.985 | 2069342 | 11489747 |
| 1s,9r-hydrastine | 20 | 0.650 | 0.403 | −0.539 | −0.849 | −0.029 | 0.359 | −0.152 | −0.041 | −0.341 | 2069343 | 11489157 |
| prednisone | 11.16 | −1.668 | 0.358 | −1.206 | −0.474 | 0.623 | −0.428 | −0.315 | −0.432 | −0.048 | 2080073 | 11467225 |
| hydrocortisone | 11.04 | −0.976 | 0.175 | −0.928 | −0.266 | 0.501 | −0.579 | −0.787 | −0.823 | −0.552 | 2080102 | 11467595 |
| cyclopiazonic acid | 20 | −1.392 | −0.353 | −1.107 | 0.863 | −0.293 | 0.033 | 0.186 | 0.163 | 0.172 | 2080198 | 11488612 |
| sisomicin | 8.94 | −0.274 | 0.411 | −1.515 | −0.962 | 1.889 | 0.746 | −0.448 | −0.337 | −0.588 | 2080587 | 11467654 |
| cycloheximide | 14.22 | −2.540 | −1.021 | −3.550 | 2.318 | −3.415 | −1.378 | −0.504 | 1.354 | −4.179 | 2080598 | 11467938 |
| cycloheximide | 20 | −2.804 | −0.704 | −3.499 | 2.759 | −2.628 | −2.123 | −1.482 | 0.688 | −5.637 | 2080598 | 11488716 |
| halofantrine | 8 | 0.132 | −0.166 | −0.174 | −0.668 | 0.012 | −0.236 | −0.508 | −0.239 | −0.969 | 2080909 | 11468179 |
| fluorometholone | 10.62 | −0.965 | −0.458 | −1.306 | −0.299 | 2.141 | −0.324 | 0.446 | 0.038 | 1.187 | 2081008 | 11467866 |
| helenine | 20 | −4.599 | −8.064 | −6.498 | −3.676 | −2.409 | −5.312 | −2.156 | −2.240 | −1.624 | 2081025 | 11488113 |
| cimetidine | 20 | −1.404 | 0.336 | −1.571 | −0.482 | 0.193 | 0.794 | −0.252 | −0.460 | 0.208 | 2081029 | 11488598 |
| amphotericin B | 4.32 | 1.210 | 0.880 | −0.790 | 0.308 | 0.005 | −0.982 | −0.046 | 0.181 | −0.507 | 2081486 | 11467558 |
| farnesol | 20 | −1.327 | 0.026 | −1.554 | −0.153 | 0.550 | −0.437 | −0.066 | 0.106 | −0.401 | 2081691 | 11489213 |
| rilmenidine | 22.2 | 0.245 | 0.135 | 0.082 | −0.589 | 0.340 | −0.042 | 0.123 | 0.319 | −0.313 | 2098610 | 11468130 |
| lithocholic acid | 10.62 | 0.256 | −1.002 | −1.419 | −0.283 | 0.353 | −0.030 | 0.002 | 0.248 | −0.491 | 2105063 | 11467944 |
| celastrol | 20 | −4.797 | −8.640 | −6.584 | −3.520 | −2.732 | −5.979 | ND | ND | ND | 2114344 | 11487966 |
| daunorubicin | 7.58 | −3.547 | −4.649 | −5.009 | −2.337 | −2.398 | −3.722 | −1.008 | −2.094 | 1.373 | 2117312 | 11467635 |
| strophanthidin | 9.88 | −0.448 | −0.236 | −0.908 | −0.175 | 0.947 | 0.287 | −0.590 | −0.729 | −0.190 | 2117332 | 11467858 |
| 4-aminoantipyrine | 4.42 | 0.530 | −1.148 | −1.470 | −4.045 | 0.564 | 0.087 | −0.182 | −0.041 | −0.450 | 2117409 | 11467573 |
| pimozide | 8.66 | 0.494 | −0.856 | −0.375 | −0.170 | 2.196 | −0.531 | −0.548 | −0.438 | −0.648 | 2117626 | 11467456 |
| pimozide | 20 | 0.146 | −0.464 | −0.447 | −0.381 | 1.432 | −0.121 | 0.722 | 0.857 | 0.386 | 2117626 | 11488842 |
| anisomycin | 15.08 | −2.835 | −1.426 | −4.623 | −1.537 | −2.958 | −2.841 | −0.353 | 1.256 | −3.545 | 2117676 | 11467560 |
| ergosterol | 20 | −0.792 | −0.762 | −1.864 | −0.489 | −0.198 | 0.386 | 0.231 | 0.420 | −0.240 | 2120750 | 11488017 |
| metixene | 12.92 | 1.221 | −1.165 | −0.786 | −0.713 | −0.834 | 0.097 | −1.190 | −1.058 | −1.236 | 2120971 | 11467639 |
| biperiden | 12.84 | 0.740 | 1.599 | −0.886 | −0.517 | 2.725 | 0.053 | −0.817 | −0.496 | −1.312 | 2121358 | 11467650 |
| iohexol | 4.88 | −0.374 | 0.395 | −1.085 | −0.970 | 0.568 | 0.080 | −0.434 | −0.312 | −0.598 | 2141046 | 11467660 |
| riboflavin | 20 | 0.590 | −0.150 | −0.732 | −0.442 | 4.295 | −0.667 | 0.626 | 0.483 | 0.774 | 2141061 | 11488476 |
| sinomenine | 20 | −0.937 | 0.495 | −1.489 | −0.657 | 0.777 | −0.117 | −0.875 | −0.565 | −1.253 | 2141064 | 11489058 |
| yohimbine | 11.28 | −0.860 | −0.305 | −1.915 | −1.197 | 0.159 | 0.699 | −0.041 | 0.169 | −0.471 | 3000363 | 11467732 |
| dantrolene | 20 | −0.497 | 0.634 | −0.490 | −0.080 | 0.712 | −1.219 | 0.160 | 0.159 | 0.210 | 3000787 | 11488996 |
| (S)-propranolol | 15.42 | 0.307 | 0.430 | −0.351 | −0.340 | −0.185 | 0.349 | 0.857 | 0.623 | 1.161 | 3002368 | 11468229 |
| furazolidone | 17.76 | 0.828 | 1.071 | −1.254 | −0.233 | −0.319 | −0.368 | −0.983 | −0.781 | −1.207 | 3043753 | 11467956 |
| furazolidone | 20 | −0.411 | 1.326 | −1.256 | −1.059 | 0.761 | 0.881 | −0.133 | −0.040 | −0.228 | 3043753 | 11488831 |
| guanabenz | 20 | −0.404 | 1.035 | −0.886 | −1.073 | 1.527 | 0.420 | −0.428 | −0.286 | −0.563 | 3044526 | 11488930 |
| trimeprazine | 8.92 | 0.760 | −0.459 | −2.221 | −0.821 | 1.314 | −2.786 | −0.942 | −0.936 | −0.778 | 3044756 | 11467990 |
| trimeprazine | 20 | −1.472 | −3.762 | −2.227 | 1.905 | −0.430 | −0.086 | −0.013 | −0.130 | 0.208 | 3044756 | 11488774 |
| guanidine carbonate | 20 | −0.835 | −0.171 | −0.683 | −0.252 | 0.401 | −1.206 | −1.142 | −1.112 | −0.993 | 3044782 | 11488665 |
| compactin | 20 | −0.454 | −0.561 | −0.569 | 0.369 | −1.309 | −0.971 | −0.154 | −0.217 | −0.027 | 3045136 | 11489795 |
| ipratropium | 20 | −0.962 | 1.230 | −0.600 | −0.508 | 1.245 | 0.049 | −0.294 | −0.091 | −0.570 | 3045140 | 11489091 |
| amoxicillin | 20 | −1.400 | −0.033 | −1.357 | −0.867 | 1.061 | 0.351 | 0.741 | 0.607 | 0.815 | 3045196 | 11487961 |
| dexamethasone | 20 | −0.392 | 0.221 | −1.719 | 0.240 | −0.512 | −0.235 | −0.339 | −0.318 | −0.250 | 3045298 | 11488942 |
| cyproterone | 20 | 0.014 | 0.856 | 0.079 | −0.962 | 1.155 | 0.548 | −0.490 | −0.461 | −0.446 | 3045655 | 11489413 |
| metaraminol | 20 | 0.107 | 1.865 | −1.268 | −0.772 | 0.313 | 0.327 | −0.589 | −0.456 | −0.745 | 3045662 | 11489358 |
| pentolinium | 10.24 | 0.213 | 1.449 | 0.254 | −0.024 | 3.608 | 0.387 | 0.521 | 0.489 | 0.444 | 3045683 | 11467336 |
| pentolinium | 20 | −0.568 | 0.247 | −0.771 | 0.209 | 0.392 | −0.703 | −1.000 | −0.840 | −1.080 | 3045683 | 11489417 |
| famotidine | 20 | −0.793 | 1.389 | −0.762 | −0.445 | 1.272 | −0.339 | 0.514 | 0.591 | 0.334 | 3045799 | 11488852 |
| halcinonide | 20 | 0.106 | −0.401 | −0.789 | 0.570 | 0.624 | −0.794 | 0.492 | 0.284 | 0.831 | 3046112 | 11489356 |
| avermectin B1 | 20 | 0.129 | −0.108 | −0.284 | −0.336 | 2.442 | 0.116 | 0.198 | 0.170 | 0.295 | 3046211 | 11488895 |
| lindane | 20 | −0.322 | 0.025 | −1.788 | −0.724 | 2.681 | 0.467 | −0.629 | −0.628 | −0.474 | 3046265 | 11489680 |
| testosterone propionate | 20 | −1.758 | 0.781 | −1.449 | −0.365 | 1.034 | −0.996 | −1.608 | −1.410 | −1.619 | 3046390 | 11489062 |
| phentolamine | 14.22 | −0.525 | −0.486 | −0.112 | −0.548 | 0.893 | −0.141 | −0.165 | −0.236 | −0.026 | 3046406 | 11467378 |
| mitomycin C | 20 | −1.520 | 0.878 | −1.986 | −0.282 | −1.187 | −1.740 | −0.139 | 0.005 | −0.430 | 3046992 | 11488736 |
| bisabolol | 20 | 0.129 | −0.136 | −1.329 | 0.164 | 1.461 | 0.068 | −0.205 | −0.275 | 0.008 | 3053888 | 11489601 |
| cedrol | 20 | −0.226 | 0.003 | −0.896 | 0.175 | 0.096 | −0.218 | −0.319 | −0.249 | −0.351 | 3053889 | 11489602 |
| aconitic acid | 20 | 0.322 | 1.106 | −0.544 | −0.030 | 0.839 | −1.099 | −0.886 | −0.951 | −0.538 | 3053891 | 11489604 |
| allopregnanolone | 20 | 0.743 | 0.263 | 0.733 | 0.732 | 1.285 | 1.264 | −0.021 | 0.077 | −0.274 | 3053987 | 11488086 |
| euphol | 20 | 0.693 | −0.327 | −0.521 | 1.283 | −0.234 | −0.662 | −0.661 | −0.754 | −0.405 | 3054028 | 11487986 |
| pinosylvin | 20 | −0.614 | 0.027 | −1.343 | −0.444 | −0.927 | 0.602 | −0.409 | −0.198 | −0.802 | 3054030 | 11488009 |
| violastyrene | 20 | −0.886 | −0.880 | −1.761 | −0.080 | −0.141 | 0.063 | −0.800 | −0.674 | −0.947 | 3054032 | 11488011 |
| nerolidol | 20 | −0.629 | −0.121 | −1.158 | −0.552 | 0.392 | −0.236 | −0.170 | −0.188 | −0.092 | 3054057 | 11489323 |
| chaulmoogric acid | 20 | 0.156 | 0.746 | −1.195 | −0.173 | 0.706 | 0.264 | −0.255 | −0.059 | −0.539 | 3054072 | 11489038 |
| stigmasterol | 20 | −0.336 | −0.213 | −1.330 | −0.621 | −0.227 | 0.442 | −0.522 | −0.467 | −0.488 | 3054095 | 11489449 |
| tigogenin | 20 | −0.452 | −0.530 | −1.580 | −0.277 | 1.151 | −0.049 | −0.118 | −0.105 | −0.177 | 3054097 | 11488093 |
| xanthopterin | 20 | −1.031 | 1.368 | −0.844 | 0.187 | 0.799 | 0.798 | −0.343 | 0.237 | −1.465 | 3054108 | 11488459 |
| anthothecol | 20 | −4.715 | −8.676 | −6.566 | −3.669 | −3.614 | −6.072 | ND | ND | ND | 3054122 | 11488041 |
| crinamine | 20 | −2.147 | 0.593 | −4.403 | 0.043 | −2.125 | −0.619 | 0.628 | 2.270 | −2.895 | 3054128 | 11488098 |
| ambelline | 20 | −0.302 | 0.086 | −1.389 | 0.246 | 0.774 | −0.311 | −0.823 | −1.011 | −0.330 | 3054129 | 11488015 |
| euphol acetate | 20 | −1.099 | 0.149 | −1.087 | 0.066 | 0.660 | −0.739 | −1.280 | −1.298 | −0.951 | 3054130 | 11488176 |
| beta-amyrin | 20 | −0.235 | 0.506 | −1.204 | −1.039 | −0.089 | 0.610 | 0.371 | 0.459 | 0.146 | 3054135 | 11488169 |
| beta-amyrin | 20 | −0.096 | 0.910 | −1.893 | −0.598 | 1.276 | 0.329 | 0.119 | 0.263 | −0.128 | 3054136 | 11489069 |
| corynanthine | 20 | 0.214 | 1.318 | −0.721 | −0.032 | 0.470 | 0.397 | −0.955 | −1.008 | −0.672 | 3054270 | 11489188 |
| ursolic acid | 20 | −2.670 | −0.787 | −2.032 | −0.737 | −0.795 | 0.191 | −0.759 | −0.718 | −0.652 | 3054523 | 11489560 |
| oleanolic acid | 20 | 0.061 | 0.521 | −1.409 | −0.480 | 0.500 | 0.866 | 0.105 | 0.081 | 0.071 | 3054572 | 11488109 |
| scandenin | 20 | −0.337 | −0.093 | −0.897 | −0.508 | 0.767 | 0.268 | −0.079 | 0.284 | −0.745 | 3054634 | 11489722 |
| cholecalciferol | 20 | 0.201 | 0.289 | −0.433 | 0.185 | 1.221 | 0.110 | −0.138 | −0.261 | 0.137 | 3054675 | 11489185 |
| deoxysappanone B 7,3′-dimethyl ether acetate | 20 | 0.301 | 0.598 | −1.919 | −0.107 | −1.894 | −1.602 | 0.097 | −0.156 | 0.544 | 3054809 | 11487995 |
| corticosterone | 11.54 | −0.693 | 0.075 | −1.282 | 0.088 | 0.126 | −0.379 | −0.358 | −0.282 | −0.446 | 3054850 | 11467580 |
| quinic acid | 20 | 0.174 | −0.385 | −0.580 | 0.145 | 1.099 | −0.402 | −0.057 | −0.065 | 0.010 | 3054971 | 11489606 |
| abietic acid | 20 | 0.225 | 0.173 | −0.406 | −0.311 | −0.126 | −0.410 | 0.268 | 0.264 | 0.229 | 3054972 | 11489314 |
| ajmalicine | 11.34 | −0.201 | 0.822 | −1.608 | −0.826 | 0.948 | 0.251 | −0.178 | −0.210 | −0.081 | 3054974 | 11467740 |
| menthone | 20 | 0.344 | 1.509 | −0.587 | −0.519 | −0.339 | 0.423 | −0.611 | −0.169 | −1.418 | 3054976 | 11488507 |
| deoxyadenosine | 20 | 0.279 | 0.471 | −0.133 | −0.458 | −0.575 | 0.271 | 0.250 | 0.366 | −0.033 | 3054980 | 11489317 |
| acacetin | 20 | −0.687 | −0.691 | −0.727 | 0.032 | 0.048 | 0.531 | −1.020 | −0.999 | −0.919 | 3054981 | 11488008 |
| mifepristone | 9.32 | −0.149 | −0.183 | −0.918 | −0.201 | 1.602 | −1.159 | 0.282 | 0.143 | 0.506 | 3055129 | 11467447 |
| pristimerol | 20 | −5.551 | −6.511 | −5.956 | −3.658 | −0.368 | −6.066 | −2.500 | −2.190 | −2.630 | 3055171 | 11488528 |
| pinosylvin methyl ether | 20 | −0.471 | −0.538 | −0.867 | −0.451 | 0.042 | 0.368 | −0.304 | −0.390 | −0.112 | 3055207 | 11488010 |
| ascorbic acid | 20 | 0.198 | 0.527 | −0.653 | 0.212 | −0.048 | −1.661 | 0.027 | −0.014 | 0.055 | 3055218 | 11489764 |
| coniine | 20 | −0.475 | 0.491 | −1.000 | −0.564 | 0.659 | −0.425 | 0.008 | 0.035 | −0.084 | 3055245 | 11489782 |
| dalbergione | 20 | −0.865 | 0.685 | −2.448 | −0.586 | −0.629 | −1.116 | −0.176 | −0.010 | −0.396 | 3055248 | 11488974 |
| acrisorcin | 20 | −4.510 | 0.461 | −2.420 | −0.408 | 0.053 | −0.132 | 0.399 | 0.810 | −0.434 | 3055280 | 11488923 |
| uvaol | 20 | −0.873 | 0.124 | −0.714 | −0.266 | 0.662 | −0.846 | −0.626 | −0.531 | −0.641 | 3055306 | 11489456 |
| loganin | 20 | −0.968 | 0.561 | −1.114 | −0.744 | 0.441 | 0.272 | −0.299 | −0.206 | −0.391 | 3055308 | 11489453 |
| bergenin | 20 | −0.657 | 0.395 | −1.153 | −0.254 | 0.647 | −0.039 | −0.338 | −0.230 | −0.443 | 3055310 | 11488416 |
| triptonide | 20 | −3.627 | −5.120 | −4.144 | −2.417 | −2.071 | −4.153 | 0.149 | −2.183 | 5.015 | 3055313 | 11488293 |
| cholic acid | 20 | −1.325 | −0.370 | −1.806 | −0.579 | 0.950 | 0.514 | −0.250 | −0.272 | −0.115 | 3055321 | 11488420 |
| thioxolone | 20 | −0.135 | −0.107 | −1.399 | −2.030 | 0.060 | −0.199 | −0.012 | −0.179 | 0.374 | 3055336 | 11488266 |
| curcumin | 20 | −0.722 | 1.107 | −2.170 | −2.198 | −0.642 | −0.468 | −0.575 | −0.300 | −0.982 | 3055363 | 11489530 |
| gangleoidin acetate | 20 | −0.001 | −0.292 | −0.705 | −0.785 | 0.114 | −0.136 | −0.602 | −0.560 | −0.509 | 3055370 | 11488979 |
| marmesin | 20 | 0.129 | 0.172 | −1.130 | −0.945 | 0.854 | 0.834 | 0.519 | 0.597 | 0.237 | 3055383 | 11488553 |
| cosmosiin | 20 | −0.513 | 0.932 | −0.677 | −0.907 | 1.120 | 0.144 | −0.613 | −0.648 | −0.382 | 3055391 | 11489568 |
| lupinine | 20 | 0.417 | 0.645 | −0.980 | 0.039 | 0.444 | −0.249 | −0.804 | −0.723 | −0.769 | 3055414 | 11488315 |
| marmesin acetate | 20 | 0.976 | 0.720 | −1.647 | −0.592 | 0.641 | 0.449 | 0.025 | 0.221 | −0.308 | 3055445 | 11489028 |
| epicoprosterol | 20 | −1.124 | 0.257 | −1.568 | −1.063 | 0.312 | −0.025 | 0.086 | 0.230 | −0.186 | 3055472 | 11489452 |
| anethole | 20 | −0.309 | 0.204 | −1.000 | −0.720 | 0.097 | −0.161 | −0.431 | −0.682 | 0.215 | 3055475 | 11488354 |
| nicotinyl tartrate | 20 | −0.406 | 1.071 | −0.727 | −0.611 | 0.550 | −0.048 | −0.218 | −0.313 | 0.057 | 3055569 | 11489546 |
| inosine | 20 | 0.264 | 0.379 | −0.337 | −0.630 | 0.851 | 0.121 | −0.339 | −0.457 | −0.056 | 3055573 | 11489755 |
| sparteine | 20 | −0.501 | 1.073 | −0.554 | 1.223 | 0.700 | 0.518 | −0.203 | −0.149 | −0.295 | 3057756 | 11488537 |
| sparteine | 20 | −0.526 | −0.130 | −1.158 | −0.676 | 1.118 | −0.037 | −1.464 | −1.473 | −1.212 | 3057756 | 11489790 |
| chlorotrianisene | 10.5 | −1.153 | 0.419 | −1.139 | −0.487 | 1.360 | 0.165 | −0.478 | −0.328 | −0.693 | 3057880 | 11467905 |
| chlorotrianisene | 20 | 0.118 | 0.354 | 0.277 | −0.360 | 0.945 | 0.794 | −0.538 | −0.382 | −0.764 | 3057880 | 11488520 |
| sulpiride | 20 | −1.524 | −0.188 | −1.375 | −0.612 | 0.562 | −0.068 | −0.227 | −0.183 | −0.265 | 3058009 | 11489240 |
| methoxamine | 18.94 | −0.871 | −0.387 | −1.537 | −0.655 | 1.380 | 1.668 | 0.022 | 0.262 | −0.467 | 3058468 | 11467683 |
| methoxamine | 20 | −1.522 | −0.205 | −1.209 | −1.270 | 1.719 | −0.125 | −0.825 | −0.756 | −0.827 | 3058468 | 11488592 |
| alpha-hyodeoxycholic acid | 20 | −1.074 | −0.154 | −0.797 | −0.678 | 0.184 | 0.106 | −0.737 | −0.620 | −0.868 | 3058484 | 11489767 |
| (−)-deguelin | 20 | −0.940 | −5.123 | −4.052 | 1.451 | 0.426 | −1.865 | −1.423 | −1.606 | −0.834 | 3058525 | 11488114 |
| estrone | 20 | 0.054 | 0.623 | −0.937 | −0.532 | 0.385 | 0.076 | −0.251 | −0.031 | −0.584 | 3058535 | 11488850 |
| ergonovine | 20 | −0.536 | 1.630 | −0.529 | 0.139 | 0.104 | 0.826 | −0.231 | −0.113 | −0.491 | 3058614 | 11487820 |
| naringin | 20 | 0.198 | 0.672 | −0.963 | 0.746 | 1.074 | 0.127 | 0.054 | 0.120 | −0.097 | 3058615 | 11489181 |
| piscidic acid | 20 | −0.160 | 0.136 | −1.141 | −0.632 | 0.364 | −0.241 | −0.512 | −0.613 | −0.255 | 3058620 | 11488023 |
| solasodine | 20 | −0.392 | 1.605 | −0.747 | −0.407 | 0.797 | 0.280 | −0.330 | −0.296 | −0.293 | 3058621 | 11488163 |
| brazilein | 20 | −1.633 | 0.713 | −0.725 | −0.110 | 1.078 | −1.301 | 1.041 | 1.169 | 0.556 | 3058622 | 11488526 |
| androsterone | 20 | 0.953 | 0.035 | 0.058 | 0.163 | 0.247 | 0.192 | 0.128 | 0.133 | 0.031 | 3058740 | 11487968 |
| spironolactone | 20 | −0.957 | 1.029 | −0.765 | −1.347 | 0.697 | −0.171 | 0.377 | 0.249 | 0.571 | 3059108 | 11489132 |
| cholestan-3-one | 20 | −1.020 | 0.144 | −1.121 | −0.585 | 0.836 | 0.435 | 0.246 | 0.144 | 0.353 | 3059164 | 11489769 |
| dioxybenzone | 16.38 | −0.704 | 0.981 | −0.414 | −0.444 | 1.241 | 0.256 | −0.840 | −0.750 | −0.900 | 3059213 | 11468046 |
| dioxybenzone | 20 | 0.295 | 0.629 | −0.314 | −0.080 | 0.459 | 0.684 | 0.924 | 0.963 | 0.741 | 3059213 | 11488848 |
| estradiol propionate | 20 | −0.359 | 0.733 | −0.310 | −1.076 | 0.717 | 0.651 | 0.042 | 0.036 | 0.052 | 3059431 | 11489252 |
| 7-oxocholesterol | 20 | 0.614 | 0.694 | 0.024 | 0.913 | 0.692 | 1.259 | −0.675 | −0.697 | −0.453 | 3059432 | 11488381 |
| estradiol acetate | 20 | 0.983 | 0.847 | −1.085 | 1.098 | 1.047 | −0.085 | 0.316 | 0.339 | 0.141 | 3059433 | 11487826 |
| kobusone | 20 | 0.754 | 0.341 | −0.669 | −0.231 | 0.715 | 0.388 | 0.879 | 0.721 | 0.952 | 3059434 | 11488131 |
| chloramphenicol | 20 | 0.340 | 0.966 | −1.003 | −0.910 | 0.096 | 0.205 | −0.747 | −1.154 | 0.219 | 3059437 | 11488700 |
| aphyllic acid | 20 | −0.337 | −0.820 | −1.304 | 0.649 | 0.837 | 1.013 | 0.694 | 0.936 | 0.045 | 3059440 | 11488541 |
| orlistat | 20 | 2.525 | 0.960 | 0.847 | 0.306 | 1.029 | −0.233 | 0.056 | −0.005 | 0.199 | 3059445 | 11489494 |
| cefdinir | 20 | 0.174 | −0.201 | −0.929 | −0.343 | 0.833 | 1.025 | −0.047 | −0.008 | −0.091 | 3059465 | 11489501 |
| ceftibuten | 20 | −0.394 | 1.208 | −0.776 | 0.264 | 1.538 | 0.063 | −0.061 | −0.153 | 0.166 | 3059791 | 11489500 |
| valsartan | 20 | −0.540 | 0.559 | 0.346 | −0.198 | 0.816 | 0.174 | −0.818 | −0.894 | −0.435 | 3059817 | 11488936 |
| vidarabine | 14.96 | −1.004 | −0.331 | −1.667 | −0.335 | 0.404 | −0.784 | −0.879 | −0.885 | −0.698 | 3060003 | 11467916 |
| idazoxan | 19.58 | −0.270 | 0.477 | −0.656 | −0.281 | −0.076 | −0.533 | 0.975 | 0.876 | 0.990 | 3060036 | 11468074 |
| estrone | 14.8 | −0.739 | 0.498 | −0.632 | 0.063 | 0.272 | 0.360 | 0.104 | −0.108 | 0.512 | 3060043 | 11468062 |
| guanabenz | 17.32 | 0.167 | 0.774 | −0.950 | −1.001 | 0.878 | 0.726 | 0.197 | −0.043 | 0.591 | 3060090 | 11467244 |
| ketoconazole | 7.52 | −0.490 | −0.467 | −0.448 | 0.304 | 1.572 | 0.406 | 0.527 | 0.701 | 0.073 | 3060108 | 11467537 |
| DO 897/99 | 9.58 | 0.519 | −1.132 | −0.446 | 0.416 | 0.511 | 0.327 | 0.520 | 0.276 | 0.924 | 3060137 | 11467707 |
| budesonide | 9.3 | −1.282 | −0.421 | −1.390 | −1.016 | 0.648 | −0.730 | 0.532 | 0.578 | 0.335 | 3060147 | 11467666 |
| iobenguane sulfate | 14.54 | 1.366 | −1.160 | −0.971 | 0.229 | −1.253 | 0.392 | 0.637 | 0.845 | 0.080 | 3060173 | 11467638 |
| (S)-methoprene | 20 | −1.056 | 0.724 | −1.068 | −0.804 | 0.171 | 0.097 | 0.202 | 0.107 | 0.341 | 3060195 | 11488521 |
| moxifloxacin | 20 | −0.374 | 0.970 | −0.915 | −0.680 | −0.326 | 0.145 | −0.720 | −0.794 | −0.386 | 3060196 | 11488339 |
| cefotaxime | 8.78 | −0.254 | 0.371 | −0.793 | −0.037 | 0.239 | −0.624 | 0.243 | 0.232 | 0.176 | 3060388 | 11467287 |
| doxepin | 14.32 | 0.584 | −0.076 | −1.279 | −0.780 | 0.478 | 1.151 | −0.746 | −0.608 | −0.886 | 3060494 | 11467411 |
| scopolamine | 13.18 | −0.789 | −0.912 | −1.072 | −0.784 | 0.081 | 0.260 | −0.078 | −0.051 | −0.121 | 3060919 | 11468025 |
| lobeline | 11.86 | −0.381 | −0.165 | −1.496 | −1.248 | 1.284 | 0.619 | −0.216 | −0.012 | −0.606 | 3061052 | 11467733 |
| 4-aminocrotonic acid | 20 | −0.667 | 1.062 | −0.862 | −0.614 | −0.398 | 0.446 | −0.542 | −0.579 | −0.360 | 3061087 | 11489289 |
| ketanserin | 7.34 | −0.527 | −0.388 | −0.448 | −0.581 | 0.083 | −0.247 | 0.275 | 0.336 | 0.093 | 3061431 | 11467540 |
| xamoterol | 11.78 | 0.153 | 0.495 | −0.899 | −0.528 | 0.274 | 0.281 | −0.178 | −0.078 | −0.359 | 3061617 | 11468071 |
| cytochalasin E | 20 | −0.980 | 2.747 | −2.467 | −0.864 | −2.109 | −3.335 | 1.353 | 1.449 | 0.973 | 3063989 | 11489056 |
| isoflupredone acetate | 9.52 | −1.329 | −0.025 | −0.188 | 0.397 | 0.011 | 0.267 | 0.181 | 0.341 | −0.221 | 3064163 | 11467154 |
| dantrolene | 12.72 | 0.272 | 0.077 | −1.537 | −0.516 | 0.214 | 0.359 | −0.769 | −0.514 | −1.138 | 3068677 | 11467439 |
| benzydamine | 12.92 | −0.074 | −0.703 | 0.120 | −0.141 | 0.426 | 0.052 | 0.560 | 0.470 | 0.639 | 3068682 | 11467445 |
| norcyclobenzaprine | 15.3 | −0.369 | −1.466 | −2.283 | −0.427 | 0.956 | −0.724 | −0.440 | −0.328 | −0.586 | 3068692 | 11467661 |
| imipenem | 13.36 | −0.973 | 0.250 | −0.777 | −0.461 | 0.790 | −0.330 | −0.495 | −0.319 | −0.755 | 3068724 | 11467667 |
| L-methionine sulfoximine | 22.2 | −0.928 | 0.202 | −1.902 | −0.703 | 0.762 | −0.412 | −0.766 | −0.620 | −0.913 | 3068727 | 11467671 |
| triflusal | 16.12 | −1.375 | −0.004 | −0.432 | −0.592 | 1.275 | −0.003 | −0.390 | −0.401 | −0.292 | 3068731 | 11467676 |
| clorsulon | 10.5 | −0.111 | −0.210 | −0.774 | −0.647 | 1.055 | −0.186 | 0.240 | 0.394 | −0.125 | 3068774 | 11467688 |
| pregnenolone | 12.64 | −0.330 | 0.824 | 0.061 | 0.542 | 1.242 | 0.404 | 0.250 | 0.185 | 0.345 | 3068775 | 11467694 |
| dihydroergotoxine | 7.1 | −0.119 | 1.009 | −0.443 | 0.046 | 0.218 | 0.576 | −0.087 | −0.053 | −0.139 | 3068781 | 11467717 |
| lincomycin | 9.84 | −0.258 | 0.766 | −0.935 | −0.749 | 0.331 | 0.288 | −0.689 | −0.716 | −0.492 | 3068878 | 11467450 |
| phenylpropanolamine | 26.46 | −0.373 | 1.781 | −0.396 | −0.650 | 0.552 | 0.947 | 0.487 | 0.541 | 0.267 | 3068879 | 11467472 |
| ascorbic acid | 22.46 | −0.040 | 0.900 | −1.280 | 0.503 | 0.350 | 0.380 | −0.141 | −0.115 | −0.170 | 3068880 | 11467473 |
| zaprinast | 14.74 | −0.634 | 1.014 | −1.911 | −0.704 | 1.895 | −0.302 | 0.224 | 0.330 | −0.027 | 3068881 | 11467483 |
| chlorprothixene | 12.66 | 0.574 | 0.645 | −0.544 | 0.089 | 1.237 | −1.373 | 0.206 | 0.112 | 0.358 | 3068882 | 11467496 |
| adenosine 5′-monophosphate | 11.52 | −1.097 | 0.061 | −1.214 | −0.915 | 1.276 | 0.104 | −0.137 | −0.195 | 0.005 | 3068883 | 11467504 |
| betamethasone | 10.2 | −0.479 | −0.371 | −1.315 | −0.688 | 0.846 | −0.822 | 0.298 | 0.178 | 0.490 | 3068885 | 11467510 |
| clofazimine | 8.44 | −1.383 | −0.999 | −1.191 | 0.552 | 1.422 | 0.320 | −0.358 | −0.184 | −0.640 | 3068886 | 11467524 |
| amikacin | 6.84 | −1.029 | −0.026 | −0.942 | −0.723 | 1.056 | 0.015 | 0.831 | 0.438 | 1.480 | 3068923 | 11467543 |
| clomiphene | 9.86 | −0.620 | 0.022 | −0.400 | −0.755 | 1.587 | 0.699 | 0.461 | 0.354 | 0.593 | 3068926 | 11467545 |
| sulfaguanidine | 18.68 | 0.216 | 2.181 | 0.059 | 0.511 | 0.379 | 0.509 | 0.738 | 0.787 | 0.437 | 3068956 | 11467158 |
| idoxuridine | 11.3 | 0.295 | 0.995 | −0.131 | −0.167 | 1.086 | 0.916 | −0.548 | −0.456 | −0.662 | 3068957 | 11467166 |
| captopril | 17.3 | 0.347 | 0.600 | −0.646 | −0.783 | 1.363 | 0.551 | 0.773 | 0.799 | 0.522 | 3068958 | 11467167 |
| cimetidine | 15.86 | −1.323 | −0.082 | −1.129 | −0.952 | 1.066 | 0.587 | 0.216 | 0.365 | −0.164 | 3068961 | 11467174 |
| betazole | 35.98 | −0.554 | 0.135 | −1.681 | −0.798 | 1.677 | −0.746 | −1.100 | −1.056 | −1.020 | 3068964 | 11467191 |
| SR-95639A | 12.32 | 0.594 | 0.858 | −1.195 | −0.810 | 0.398 | 0.777 | −0.413 | −0.088 | −0.996 | 3068973 | 11467554 |
| butoconazole | 9.72 | 2.031 | 1.793 | 0.124 | −0.404 | −0.267 | 0.908 | 0.378 | 0.515 | 0.017 | 3068976 | 11467556 |
| homatropine | 14.52 | −0.696 | −0.538 | −1.372 | −0.284 | 0.546 | −0.371 | −0.394 | −0.753 | 0.358 | 3068999 | 11467210 |
| lynestrenol | 14.06 | 0.501 | 1.467 | 0.375 | −0.592 | 0.575 | 0.855 | −0.669 | −0.460 | −1.001 | 3069004 | 11467243 |
| acenocoumarol | 11.32 | −0.433 | 0.530 | −0.108 | −0.353 | 1.383 | −0.329 | 0.099 | −0.004 | 0.254 | 3069006 | 11467258 |
| carcinine | 21.96 | −0.003 | 0.853 | −2.838 | −0.375 | 1.637 | 0.716 | −0.111 | 0.006 | −0.328 | 3069011 | 11467570 |
| metanephrine | 20.28 | −0.010 | −0.156 | −2.109 | −0.722 | −0.114 | −0.413 | −0.180 | −0.322 | 0.095 | 3069040 | 11467270 |
| erythromycin | 5.46 | 0.404 | 0.204 | 0.112 | 0.152 | −0.110 | 0.376 | 0.058 | 0.212 | −0.314 | 3069044 | 11467299 |
| josamycin | 4.84 | 0.504 | −0.793 | −0.582 | −0.902 | 0.157 | −0.322 | −0.299 | −0.561 | 0.248 | 3069045 | 11467302 |
| neomycin | 6.22 | 0.823 | −0.207 | −0.526 | −0.640 | 0.503 | −0.498 | 1.110 | 1.006 | 1.055 | 3069046 | 11467306 |
| dihydrostreptomycin | 6.86 | 0.836 | −1.306 | 0.190 | 0.027 | 0.243 | −0.860 | 1.001 | 0.969 | 0.835 | 3069047 | 11467307 |
| cyclosporine | 3.32 | 0.145 | 1.037 | −1.574 | −0.276 | 0.751 | −0.231 | −0.739 | −0.614 | −0.858 | 3069049 | 11467583 |
| carbimazole | 21.48 | −0.940 | 0.258 | −0.673 | 0.204 | 0.681 | 0.472 | −0.272 | −0.322 | −0.113 | 3069053 | 11467587 |
| carbimazole | 20 | 0.118 | −0.909 | −0.740 | −0.239 | 0.121 | −0.773 | −0.304 | −0.065 | −0.670 | 3069053 | 11489067 |
| tranylcypromine | 30.04 | −0.359 | 1.283 | −1.388 | −0.636 | 0.610 | 0.662 | −0.967 | −0.760 | −1.234 | 3069074 | 11467321 |
| aceclofenac | 11.3 | 0.026 | 0.373 | −1.348 | −1.058 | 0.923 | 0.188 | −0.732 | −0.562 | −0.965 | 3069075 | 11467323 |
| tiratricol, 3,3′,5-triiodothyroacetic acid | 6.44 | 0.899 | 0.450 | −1.189 | −0.933 | 2.224 | 0.133 | −1.203 | −1.019 | −1.379 | 3069077 | 11467350 |
| pyrantel tartrate | 11.22 | 0.330 | 0.280 | −1.214 | −1.028 | 1.579 | −0.306 | −0.292 | −0.094 | −0.684 | 3069079 | 11467360 |
| hydroxytacrine | 19.02 | −0.957 | 0.732 | −1.910 | −0.265 | 0.783 | −0.913 | −1.214 | −1.177 | −1.051 | 3069083 | 11467596 |
| gamma-lumicolchicine | 10.02 | 0.202 | −0.270 | −1.685 | −0.866 | 0.216 | 0.280 | −0.666 | −0.548 | −0.785 | 3069088 | 11467601 |
| indapamide | 11.44 | −0.066 | −0.697 | −0.306 | −0.256 | 1.075 | 0.210 | 0.864 | 0.591 | 1.227 | 3069113 | 11467368 |
| griseofulvin | 11.28 | −0.976 | −0.643 | −1.970 | −0.157 | 0.371 | 2.694 | 0.979 | 0.925 | 0.847 | 3069117 | 11467374 |
| prostaglandin F2a | 8.42 | −0.629 | −0.180 | −0.518 | −0.032 | 0.858 | −0.834 | −0.065 | −0.110 | 0.041 | 3069122 | 11467608 |
| metrizamide | 5.06 | 0.533 | −0.167 | −0.578 | −0.451 | 0.038 | 0.184 | 0.437 | 0.422 | 0.384 | 3069124 | 11467611 |
| scopolamin-N-oxide | 12.52 | −0.769 | 0.095 | −0.263 | −0.602 | 1.578 | −0.112 | 0.551 | 0.511 | 0.474 | 3069144 | 11467380 |
| ceforanide | 7.7 | −1.144 | −0.981 | −0.084 | 0.103 | 0.456 | −1.259 | 0.301 | 0.292 | 0.267 | 3069161 | 11467618 |
| pantothenic acid | 18.24 | 0.674 | 0.577 | −0.424 | −0.292 | −0.524 | 0.587 | 0.065 | 0.048 | 0.088 | 3069162 | 11467620 |
| vincamine | 11.28 | 0.430 | −0.634 | −0.578 | −1.235 | 0.466 | 0.601 | −0.978 | −0.922 | −0.894 | 3069234 | 11467419 |
| convolamine | 13.1 | −1.139 | −0.271 | −0.633 | −0.586 | 0.608 | 0.353 | −0.198 | 0.121 | −0.799 | 3069519 | 11467744 |
| scoulerine | 12.22 | 0.205 | −0.905 | −1.969 | −0.523 | −0.546 | −0.599 | 0.570 | 0.842 | −0.080 | 3069520 | 11467749 |
| ajmaline | 12.26 | 0.361 | −0.375 | −1.763 | −1.166 | 0.605 | −0.215 | −0.018 | −0.022 | −0.002 | 3069521 | 11467750 |
| piperlongumine | 12.6 | −5.322 | −8.479 | −6.177 | −3.952 | −3.072 | −4.976 | −2.267 | −3.189 | 0.033 | 3069522 | 11467752 |
| cinchonine | 13.58 | −1.819 | −0.404 | −1.353 | −0.976 | 1.139 | −0.482 | −0.336 | −0.187 | −0.555 | 3069523 | 11467756 |
| chrysene-1,4-quinone | 15.48 | −5.351 | −6.071 | −3.766 | −3.925 | −2.891 | −3.568 | −1.591 | −1.995 | −0.459 | 3069524 | 11467763 |
| sparteine | 17.06 | −0.903 | −0.294 | −1.355 | −0.426 | 1.035 | −0.096 | −0.182 | −0.071 | −0.373 | 3069525 | 11467766 |
| stachydrine | 27.74 | −0.162 | 0.242 | −0.763 | −0.289 | 0.483 | 0.223 | −0.115 | −0.139 | −0.038 | 3069526 | 11467770 |
| folic acid | 9.06 | −1.015 | −0.238 | −1.467 | −0.636 | −0.087 | 0.136 | −0.215 | −0.223 | −0.167 | 3069527 | 11467775 |
| retrorsine | 11.38 | 0.133 | 0.249 | −1.368 | −0.642 | 0.144 | −0.062 | 0.139 | −0.111 | 0.612 | 3069528 | 11467785 |
| solanine | 4.6 | −1.799 | −0.277 | −0.507 | −0.194 | −0.210 | −0.677 | −0.290 | −0.450 | 0.110 | 3069529 | 11467788 |
| N-acetyl-DL-homocysteine thiolactone | 25.12 | −0.895 | 1.819 | −0.899 | 0.186 | 0.742 | 1.140 | 0.525 | 0.564 | 0.332 | 3069530 | 11467792 |
| betonicine | 24.98 | −0.540 | 2.314 | −1.044 | 0.495 | 1.417 | 1.243 | 0.369 | 0.438 | 0.148 | 3069532 | 11467796 |
| halcinonide | 8.8 | −0.077 | −0.081 | −1.446 | −0.172 | 1.375 | 0.020 | 0.738 | 0.740 | 0.592 | 3069534 | 11467803 |
| 6-furfurylaminopurine | 18.58 | 0.152 | 0.370 | −1.208 | −0.508 | 0.625 | 0.598 | 0.156 | 0.275 | −0.115 | 3069535 | 11467807 |
| vitexin | 9.26 | 0.412 | 0.151 | −1.527 | −0.572 | 1.178 | 0.696 | −0.005 | −0.067 | 0.098 | 3069536 | 11467809 |
| delcorine | 8.34 | −0.164 | −0.303 | −1.152 | −0.587 | 0.794 | 0.539 | −0.350 | −0.184 | −0.626 | 3069538 | 11467812 |
| hippeastrine | 12.68 | −1.004 | 0.183 | −2.293 | −1.307 | 0.272 | 0.165 | 0.635 | 1.137 | −0.496 | 3069539 | 11467823 |
| delsoline | 8.56 | −1.275 | 0.197 | −1.028 | −0.561 | 0.652 | 0.315 | −0.043 | 0.081 | −0.271 | 3069540 | 11467827 |
| austricine | 15.24 | 0.286 | 0.150 | −1.755 | −0.335 | 0.945 | 0.253 | 0.007 | 0.009 | 0.000 | 3069541 | 11467829 |
| heliotrine | 12.76 | −0.856 | 0.023 | −1.575 | −0.855 | 0.383 | 0.203 | −0.482 | −0.535 | −0.292 | 3069542 | 11467832 |
| lycorine | 13.92 | −3.004 | −0.761 | −5.005 | −1.958 | −2.127 | −1.766 | 0.147 | 1.859 | −3.343 | 3069543 | 11467834 |
| ungerine | 12.14 | −0.622 | −0.258 | −1.642 | −0.628 | 1.505 | 0.114 | −0.341 | −0.323 | −0.310 | 3069544 | 11467837 |
| 3-alpha-hydroxy-5-beta-androstan-17-one | 13.78 | −1.063 | 0.314 | −1.534 | −1.010 | 1.400 | 0.285 | 0.078 | 0.502 | −0.786 | 3069570 | 11467845 |
| finasteride | 10.74 | −1.294 | 0.938 | −1.123 | −1.106 | 1.464 | 0.352 | 0.005 | 0.035 | −0.043 | 3069574 | 11467865 |
| hecogenin | 9.28 | 1.171 | 0.661 | −0.341 | −0.048 | −0.406 | −0.480 | −0.354 | −0.368 | −0.262 | 3069577 | 11467878 |
| nadide | 6.02 | 0.517 | 0.796 | −1.254 | −0.741 | 0.435 | 0.922 | −0.630 | −0.545 | −0.688 | 3069579 | 11467889 |
| glycopyrrolate | 12.56 | 0.316 | 0.546 | −1.267 | −0.600 | −0.012 | 0.260 | −0.876 | −0.881 | −0.703 | 3069580 | 11467894 |
| cefamandole | 8.64 | −0.386 | 0.568 | −0.841 | −0.849 | 0.954 | 0.572 | −0.695 | −0.355 | −1.250 | 3069581 | 11467895 |
| mevalonic-DL-acid lactone | 30.74 | −0.251 | 0.302 | −1.044 | −0.183 | 0.874 | −0.252 | −0.456 | −0.483 | −0.317 | 3069582 | 11467898 |
| furaltadone | 12.34 | −1.079 | −0.861 | −1.752 | −0.722 | 0.306 | 0.213 | −0.721 | −0.631 | −0.766 | 3069584 | 11467912 |
| norgestrel | 12.8 | 0.768 | 0.277 | −1.551 | −0.762 | 0.452 | 0.416 | −0.446 | −0.533 | −0.192 | 3069624 | 11467921 |
| clobetasol | 8.56 | −0.047 | −1.143 | 0.131 | 0.360 | −0.090 | 0.025 | 0.670 | 0.396 | 1.103 | 3069627 | 11467929 |
| methazolamide | 16.92 | 1.656 | 1.942 | −1.025 | 0.011 | 0.229 | 0.663 | −0.019 | 0.115 | −0.316 | 3069629 | 11467950 |
| methazolamide | 20 | −0.127 | 0.831 | −1.451 | −0.345 | 0.064 | 1.267 | −0.686 | −0.545 | −0.852 | 3069629 | 11489359 |
| amiprilose | 13.1 | −0.912 | −0.145 | −1.655 | −0.638 | 0.071 | 0.015 | −0.486 | −0.407 | −0.550 | 3069693 | 11467993 |
| rolitetracycline | 7.58 | −1.412 | 0.053 | −1.083 | −0.670 | 0.192 | −0.056 | −0.689 | −0.716 | −0.496 | 3069696 | 11467997 |
| (+)-levobunolol | 13.72 | 0.104 | 0.501 | −1.675 | −0.908 | 1.585 | 0.040 | −0.941 | −0.728 | −1.186 | 3069698 | 11468005 |
| 5-L-methylhydantoin | 35.06 | −0.181 | 0.072 | −0.665 | −0.661 | 0.689 | −0.258 | −0.094 | −0.033 | −0.196 | 3069699 | 11468008 |
| 5-D-methylhydantoin | 35.06 | −0.715 | −0.277 | −1.421 | −1.018 | 1.190 | 0.225 | −0.685 | −0.586 | −0.763 | 3069700 | 11468012 |
| iopamidol | 5.14 | −0.303 | 0.059 | −0.538 | −0.213 | 0.029 | 0.503 | 0.149 | 0.000 | 0.425 | 3069701 | 11468019 |
| diloxanide furoate | 12.18 | −0.260 | 0.233 | −0.622 | 0.686 | 0.417 | 0.122 | −0.539 | −0.468 | −0.588 | 3069737 | 11468051 |
| (+)-isoproterenol | 11.06 | 0.097 | 0.441 | −0.943 | −0.800 | −0.379 | 0.420 | −0.104 | −0.256 | 0.228 | 3069738 | 11468059 |
| (−)-MK 801 | 18.08 | −1.101 | 0.842 | −0.480 | −0.786 | 0.889 | −0.160 | −0.241 | −0.373 | 0.069 | 3069740 | 11468083 |
| dehydroisoandosterone 3-acetate | 12.1 | −0.547 | −0.578 | 0.116 | −0.244 | −0.010 | −0.301 | 0.339 | 0.256 | 0.426 | 3069741 | 11468085 |
| florfenicol | 11.16 | −0.049 | 0.713 | 0.263 | −0.753 | 0.922 | −0.264 | 1.435 | 0.522 | 3.009 | 3069742 | 11468103 |
| deoxycorticosterone | 12.1 | −0.252 | −0.753 | −0.088 | −0.274 | 0.606 | −0.457 | 0.547 | 0.244 | 1.057 | 3069771 | 11468105 |
| reserpinic acid | 9.98 | −0.288 | 0.045 | −0.660 | −0.289 | 1.085 | 0.975 | 0.329 | 0.302 | 0.313 | 3069774 | 11468132 |
| beta-sitosterol | 9.64 | −0.914 | 0.405 | −0.874 | −0.600 | 1.323 | 0.231 | 0.161 | 0.153 | 0.138 | 3069775 | 11468133 |
| harpagoside | 8.08 | −0.166 | 0.841 | 0.109 | 0.652 | 1.731 | −1.270 | −0.257 | −0.311 | −0.108 | 3069776 | 11468136 |
| betulin | 9.04 | −0.544 | −0.470 | −0.058 | 0.806 | 0.085 | 0.044 | 1.205 | 1.094 | 1.194 | 3069777 | 11468138 |
| pizotifen | 9.32 | 0.473 | 0.797 | −0.447 | −0.455 | 0.707 | 0.085 | 0.189 | 0.243 | 0.025 | 3069778 | 11468140 |
| cefalonium | 8.7 | −1.015 | −0.174 | −0.231 | −0.323 | 1.280 | 0.133 | 0.445 | 0.366 | 0.505 | 3069779 | 11468144 |
| zuclopenthixol | 9.98 | −0.709 | −0.654 | 0.634 | 0.549 | 0.770 | 0.195 | −0.953 | −1.065 | −0.558 | 3069780 | 11468146 |
| alfadolone | 10.24 | 0.943 | 0.045 | −0.692 | 0.010 | 0.165 | 0.237 | 1.053 | 0.938 | 1.068 | 3069781 | 11468149 |
| epitiostanol | 13.06 | −0.581 | 1.397 | 0.048 | −0.428 | 0.546 | 0.359 | 0.323 | 0.423 | 0.049 | 3069782 | 11468154 |
| etofenamate | 10.84 | −0.983 | −0.728 | −0.523 | −0.727 | 1.003 | −0.030 | 0.035 | −0.126 | 0.343 | 3069806 | 11468162 |
| isometheptene | 11.38 | −0.117 | −0.517 | −0.666 | −0.648 | 0.842 | −0.285 | 0.520 | 0.158 | 1.154 | 3069807 | 11468171 |
| articaine | 14.06 | −0.810 | −0.193 | −0.378 | −0.606 | 0.151 | −0.079 | 0.362 | 0.297 | 0.403 | 3069809 | 11468180 |
| methyldopate | 16.72 | −0.770 | −0.047 | −0.084 | −1.190 | 0.669 | −0.393 | 1.225 | 0.818 | 1.820 | 3069810 | 11468186 |
| levocabastine | 9.52 | −0.450 | −0.773 | 0.193 | 0.002 | 0.662 | −0.168 | 1.274 | 0.986 | 1.613 | 3069811 | 11468187 |
| etomidate | 16.38 | 0.764 | 0.978 | −0.504 | −0.091 | −1.259 | 1.112 | −0.453 | −0.436 | −0.417 | 3069812 | 11468189 |
| sertaconazole | 9.14 | −0.035 | 0.094 | −0.154 | 1.817 | −0.641 | 0.441 | −0.590 | −0.300 | −1.083 | 3069813 | 11468193 |
| quinethazone | 13.8 | 1.510 | −0.153 | 0.508 | 0.716 | −0.725 | −0.649 | −0.313 | −0.263 | −0.367 | 3069814 | 11468198 |
| trifluridine | 13.5 | 0.052 | 0.174 | −0.816 | −1.232 | 0.119 | 0.151 | −1.701 | −1.587 | −1.618 | 3069815 | 11468204 |
| propoxycaine | 13.58 | 0.868 | −0.056 | 0.039 | −0.150 | 0.295 | −0.699 | −0.235 | −0.114 | −0.444 | 3069816 | 11468207 |
| naftifine | 13.92 | 0.278 | 0.226 | −0.084 | −0.395 | 0.991 | 0.645 | −0.742 | −0.568 | −0.970 | 3069817 | 11468211 |
| imidurea | 10.3 | 0.297 | 0.164 | 0.181 | −0.125 | −0.256 | 0.667 | −0.061 | −0.111 | 0.035 | 3069853 | 11468219 |
| 2-chloropyrazine | 34.92 | −0.139 | −0.153 | −0.798 | −0.280 | −0.218 | −0.257 | −0.258 | −0.482 | 0.245 | 3069855 | 11468235 |
| (−)-adenosine 3′,5′-cyclic monophosphate | 12.16 | 0.242 | 0.438 | −0.410 | −0.235 | 0.098 | 0.005 | −0.499 | −0.260 | −0.902 | 3069858 | 11468240 |
| ramipril | 9.6 | 0.302 | −0.010 | −0.370 | −0.259 | 0.586 | 0.625 | 0.421 | 0.319 | 0.546 | 3069861 | 11468255 |
| parbendazole | 16.18 | 0.211 | −1.119 | −1.477 | 0.418 | −1.879 | −2.234 | 1.379 | 1.344 | 1.181 | 3069862 | 11468258 |
| saquinavir | 5.96 | 0.611 | −0.601 | 0.686 | 0.592 | −0.447 | −0.484 | 0.787 | 0.752 | 0.692 | 3069863 | 11468262 |
| silybin | 20 | 0.319 | 0.512 | −1.445 | −2.599 | 0.594 | 0.150 | −0.560 | −0.811 | 0.093 | 3076175 | 11489510 |
| geneticin | 20 | 0.063 | 0.069 | −1.566 | −1.021 | 0.490 | 0.702 | −0.980 | −0.878 | −1.047 | 3077146 | 11487848 |
| secnidazole | 20 | −0.544 | −0.336 | −0.692 | −0.644 | 0.062 | 0.347 | −0.161 | −0.213 | −0.068 | 3077147 | 11487849 |
| valeryl salycilate | 20 | 0.248 | −0.528 | 0.039 | −0.125 | 1.754 | 0.323 | 0.014 | −0.418 | 0.834 | 3077148 | 11487976 |
| 2,3-dihydroxy-4-methoxy-4′- | 20 | −1.074 | −0.506 | −1.977 | −0.234 | 0.542 | −0.089 | −1.228 | −1.322 | −0.835 | 3077173 | 11488106 |
| ethoxybenzophenone | | | | | | | | | | | | |
| apigenin | 20 | 0.805 | −0.036 | −0.257 | −1.267 | 0.836 | 0.721 | 0.040 | −0.042 | 0.144 | 3077174 | 11488107 |
| sappanone A 7-methyl ether | 20 | −0.105 | −1.089 | −2.259 | −0.358 | −1.493 | −1.126 | 1.427 | 1.337 | 1.267 | 3077175 | 11488108 |
| koparin | 20 | −0.432 | 0.138 | −0.805 | −2.598 | 0.782 | 0.261 | −1.520 | −1.335 | −1.652 | 3077176 | 11488115 |
| avocadynone | 20 | −0.153 | −1.034 | −0.381 | −0.221 | 0.712 | −0.441 | 0.381 | 0.071 | 0.867 | 3077177 | 11488116 |
| agelasine | 20 | −0.071 | −1.101 | −0.493 | −0.534 | 1.060 | 0.411 | 0.091 | 0.225 | −0.257 | 3077178 | 11488125 |
| methyl everninic acid | 20 | −1.028 | 2.351 | −0.529 | 0.643 | 0.181 | 0.429 | −0.325 | −0.291 | −0.297 | 3077346 | 11488220 |
| 4′-demethylepipodophyllotoxin | 20 | −0.436 | 0.522 | −2.699 | −0.756 | −1.107 | −1.030 | 1.666 | 1.806 | 1.098 | 3077356 | 11488261 |
| avocadene | 20 | −0.025 | 0.936 | −0.545 | −0.147 | 0.967 | −0.088 | 0.385 | 0.303 | 0.448 | 3078269 | 11488534 |
| zolmitriptan | 20 | −0.161 | 1.349 | −0.543 | 0.585 | 1.164 | 0.806 | 1.249 | 1.604 | 0.283 | 3078270 | 11488544 |
| 3alpha-hydroxy-3-deoxyangolensic acid methyl | 20 | 1.034 | 1.264 | −0.370 | 0.251 | 1.295 | −1.732 | −0.196 | −0.177 | −0.212 | 3078271 | 11488564 |
| ester | | | | | | | | | | | | |
| mesna | 20 | −0.680 | 0.889 | −1.600 | −0.699 | 0.932 | 0.598 | −0.900 | −0.827 | −0.887 | 3078272 | 11488568 |
| baeomycesic acid | 20 | −0.434 | 0.400 | −0.619 | −1.694 | 0.844 | 0.171 | 0.192 | 0.350 | −0.188 | 3078273 | 11488609 |
| L-phenylalaninol | 20 | −0.379 | 0.911 | −0.205 | −0.043 | 1.158 | −0.858 | −0.124 | −0.071 | −0.233 | 3078274 | 11488646 |
| I-alaninol | 20 | −0.353 | 0.737 | −0.342 | −0.428 | 0.242 | 0.301 | −0.652 | −0.555 | −0.738 | 3078275 | 11488647 |
| carbadox | 20 | −0.905 | −0.297 | −1.334 | −1.394 | −0.079 | −0.250 | −0.802 | −0.599 | −1.076 | 3078276 | 11488649 |
| apramycin | 20 | −1.979 | 1.018 | −0.808 | −0.407 | −0.105 | 0.223 | 0.477 | 0.413 | 0.494 | 3078277 | 11488650 |
| 5-fluoro-5′-deoxyuridine | 20 | 0.242 | 0.603 | −2.176 | −0.828 | 0.337 | 0.338 | −0.336 | −0.284 | −0.387 | 3078281 | 11488713 |
| pyrocatechuic acid | 20 | −1.076 | 1.015 | −1.800 | −1.204 | 1.147 | −0.018 | −0.386 | −0.324 | −0.367 | 3078331 | 11488902 |
| bisabolol | 20 | 0.300 | 0.982 | 0.031 | −0.234 | 0.061 | 0.738 | −0.963 | −0.983 | −0.690 | 3078456 | 11488307 |
| sertraline | 20 | 0.279 | −1.302 | −1.905 | 0.420 | 0.090 | −1.121 | −1.068 | −1.476 | 0.020 | 3078457 | 11488309 |
| ginkgolic acid | 20 | −0.686 | 0.489 | −0.461 | −0.905 | −0.116 | 0.587 | −0.062 | −0.315 | 0.505 | 3078458 | 11488330 |
| alverine citrate | 20 | 0.885 | −2.140 | −0.018 | 0.433 | 1.116 | −0.433 | −0.238 | −0.098 | −0.442 | 3078459 | 11488396 |
| cefditorin pivoxil | 20 | −0.219 | 1.534 | −0.718 | 0.245 | 0.426 | 0.200 | 0.238 | 0.100 | 0.440 | 3078460 | 11488463 |
| 4-aminoethylbenzenesulfonyl fluoride | 20 | −0.304 | 1.073 | −1.406 | −0.356 | 1.081 | −0.485 | −0.016 | −0.045 | 0.018 | 3078461 | 11488474 |
| sodium fluoroacetate | 20 | −0.906 | 0.006 | −1.379 | −0.784 | 0.613 | 0.653 | −0.009 | 0.206 | −0.459 | 3078462 | 11488480 |
| ethyl everninate | 20 | −1.061 | 0.492 | −1.302 | −0.860 | 0.125 | 0.122 | −0.428 | −0.131 | −0.980 | 3078463 | 11488500 |
| 7-oxocallitrisic acid, methyl ester | 20 | 1.266 | −0.302 | 0.111 | 2.079 | 0.162 | −0.923 | −0.876 | −0.457 | −1.555 | 3079211 | 11489276 |
| cadaverine | 20 | −1.003 | 0.066 | −0.915 | −0.730 | 0.160 | −0.135 | −0.553 | −0.667 | −0.205 | 3079212 | 11489303 |
| S-(1,2-dicarboxyethyl)glutathione | 20 | −0.049 | −0.160 | −0.635 | 0.038 | 0.258 | −0.411 | −0.364 | −0.400 | −0.219 | 3079213 | 11489306 |
| glycylleucylphenylalanine | 20 | −0.254 | 0.418 | −0.344 | −0.774 | 0.973 | −0.182 | −0.095 | 0.033 | −0.322 | 3079214 | 11489311 |
| L-leucyl-L-alanine | 20 | −0.600 | 0.475 | −0.320 | −0.307 | 0.301 | −0.861 | 0.365 | 0.465 | 0.093 | 3079215 | 11489315 |
| cosmosiin | 20 | 0.141 | −0.890 | −0.801 | 0.053 | −0.052 | −0.009 | −0.433 | −0.287 | −0.611 | 3079222 | 11489447 |
| mercaptamine | 20 | −0.271 | 0.601 | −0.085 | −0.724 | −0.661 | 0.320 | −1.175 | −0.980 | −1.294 | 3079223 | 11489483 |
| rhodocladonic acid | 20 | −0.416 | 1.670 | −1.057 | 0.068 | 0.733 | 1.150 | 0.294 | 0.393 | 0.072 | 3079224 | 11489503 |
| lupanyl acid | 20 | −0.245 | 0.049 | −0.734 | 0.351 | 0.610 | 0.451 | 0.585 | 0.578 | 0.507 | 3079225 | 11489504 |
| desoxypeganine | 20 | −0.281 | 0.956 | 1.441 | 1.046 | 0.647 | 0.372 | 0.631 | 0.621 | 0.556 | 3079226 | 11489506 |
| imidacloprid | 20 | −0.121 | −0.094 | −0.893 | −0.019 | 0.075 | 0.607 | −0.294 | −0.214 | −0.368 | 3079227 | 11489508 |
| theanine | 20 | −0.613 | 0.157 | −0.851 | −0.557 | 0.615 | 0.244 | −1.153 | −1.209 | −0.778 | 3079228 | 11489509 |
| 3,4-dihydroxycarane | 20 | 0.448 | −0.280 | −0.697 | −0.659 | 0.697 | 0.475 | −1.265 | −0.951 | −1.620 | 3079229 | 11489517 |
| limonin | 20 | −0.818 | −0.078 | −1.564 | −0.668 | 1.165 | 0.042 | −0.197 | −0.317 | 0.127 | 3079231 | 11489544 |
| 7-methoxychromone | 20 | −0.999 | −0.063 | −0.963 | −0.875 | 0.919 | 0.511 | −0.759 | −0.818 | −0.451 | 3079232 | 11489563 |
| methyl orsellinate | 20 | −0.008 | 0.487 | 0.148 | −0.632 | 1.212 | 0.573 | −1.129 | −1.000 | −1.146 | 3079279 | 11489567 |
| (2R,3R)-(−)-epiafzelechin | 20 | −0.719 | 0.986 | −0.486 | −3.118 | 1.019 | 0.166 | −0.497 | −0.559 | −0.225 | 3079280 | 11489571 |
| anhydroglucose | 20 | 0.168 | 0.059 | 0.041 | −0.480 | 0.238 | 0.688 | −0.046 | 0.322 | −0.745 | 3079366 | 11489627 |
| amitraz | 20 | −1.025 | 0.305 | −1.572 | −0.741 | 0.867 | 0.292 | −0.526 | −0.299 | −0.814 | 3079387 | 11489059 |
| 12-methoxy-4,4-bisnor-5alpha-8,11,13- | 20 | −1.078 | 0.068 | −2.017 | −0.840 | 0.559 | −0.087 | −0.375 | −0.350 | −0.270 | 3079388 | 11489071 |
| podocarpatrien-3-ol | | | | | | | | | | | | |
| iriflophenone trimethyl ether | 20 | 0.717 | −0.605 | −0.380 | 0.248 | −0.015 | 0.032 | −1.156 | −1.252 | −0.696 | 3079389 | 11489651 |
| dihydrorobustic acid | 20 | −0.721 | 2.345 | −0.780 | 0.000 | −0.115 | 0.757 | −0.836 | −0.849 | −0.701 | 3080393 | 11489739 |
| 2-methyl-5,7,8-trimethoxyisoflavone | 20 | −0.656 | 1.653 | −0.531 | 0.306 | −0.371 | 1.063 | 0.495 | 0.629 | 0.081 | 3080394 | 11489743 |
| cholestane | 20 | 0.941 | 0.220 | −1.057 | −0.945 | 0.155 | 0.043 | −0.678 | −0.651 | −0.640 | 3080395 | 11489777 |
| diprotin B | 20 | −0.639 | 0.330 | −0.589 | 0.153 | 0.689 | −1.537 | −0.522 | −0.682 | −0.126 | 3080396 | 11489806 |
| benzamil | 12.5 | 0.375 | 0.682 | −1.629 | −1.018 | 0.723 | 0.865 | −0.204 | −0.071 | −0.434 | 3103678 | 11467805 |
| parthenolide | 16.1 | −4.944 | −7.991 | −6.260 | −3.801 | −1.572 | −4.930 | −0.072 | −0.221 | 0.260 | 3103826 | 11467698 |
| protoveratrine B | 20 | 0.708 | 0.029 | 0.904 | 0.942 | 0.244 | −1.085 | 0.018 | 0.058 | −0.096 | 3172708 | 11488626 |
| cefotaxime | 20 | −1.283 | 0.479 | −2.276 | −0.276 | 1.000 | 0.366 | −0.379 | −0.450 | −0.214 | 3172713 | 11487935 |
| pralidoxime | 29.16 | 0.060 | 0.594 | −0.520 | 0.056 | −0.098 | 0.914 | 0.502 | 0.438 | 0.523 | 3172714 | 11468091 |
| pralidoxime | 20 | −1.011 | 0.365 | −0.372 | −0.513 | −0.152 | 0.329 | −1.793 | −1.380 | −2.309 | 3172714 | 11488737 |
| nitrofural | 20.18 | 0.584 | 1.443 | −0.852 | −0.518 | 0.446 | 0.342 | −0.260 | −0.010 | −0.750 | 3172716 | 11467640 |
| nitrofurazone | 20 | 0.544 | 1.199 | −0.617 | −0.185 | 2.173 | 0.785 | −0.199 | 0.038 | −0.643 | 3172716 | 11488473 |
| gentamicin sulfate | 20 | −0.804 | −0.234 | −0.150 | −1.213 | 0.369 | −0.896 | −0.117 | −0.267 | 0.188 | 3172720 | 11488504 |
| cafestol | 20 | 0.025 | 0.332 | 1.124 | 1.283 | 0.832 | 1.374 | −0.833 | −0.385 | −1.543 | 3172723 | 11489427 |
| cantharidin | 20.38 | −4.714 | −6.628 | −5.122 | −2.722 | −2.786 | −5.104 | 0.217 | 0.144 | 0.310 | 3172726 | 11468033 |
| cantharidin | 20 | −4.063 | −6.195 | −5.729 | −2.513 | −2.764 | −5.284 | 1.196 | 0.477 | 2.498 | 3172726 | 11488446 |
| betulin | 20 | −0.184 | 0.187 | −1.669 | −0.008 | 0.633 | 0.150 | 0.570 | 0.598 | 0.442 | 3172727 | 11488436 |
| methomyl | 20 | 1.361 | −0.211 | −1.481 | −0.493 | 0.231 | −0.007 | −0.703 | −0.805 | −0.317 | 3172728 | 11489688 |
| kinetin riboside | 20 | −1.883 | −2.070 | −4.309 | −1.350 | −2.722 | −2.612 | 0.882 | 0.853 | 0.750 | 3172730 | 11489269 |
| clarithromycin | 20 | −0.763 | −0.911 | −1.086 | 0.434 | 0.293 | −1.095 | −2.161 | −2.046 | −1.928 | 3172732 | 11489484 |
| carminic acid | 20 | −0.905 | −0.059 | −1.295 | −0.378 | 0.083 | −0.243 | −0.727 | −0.662 | −0.671 | 3172733 | 11488426 |
| protoveratrine A | 20 | 0.243 | 1.266 | −0.100 | 0.394 | −0.267 | 1.155 | −1.106 | −0.978 | −1.108 | 3172845 | 11488377 |
| ketorolac | 10.62 | −0.773 | −0.629 | −0.563 | 0.136 | 0.482 | 0.315 | 0.096 | −0.072 | 0.412 | 3172846 | 11468077 |
| ketorolac | 20 | −0.363 | −0.259 | −0.053 | −0.825 | 0.939 | −0.707 | −0.344 | −0.468 | −0.033 | 3172846 | 11489414 |
| nicotine | 20 | −0.093 | 0.808 | −1.228 | −0.423 | 0.503 | 0.348 | −1.360 | −1.032 | −1.705 | 3172849 | 11489029 |
| dexamethasone acetate | 20 | −1.518 | 0.307 | 0.058 | −0.439 | 0.067 | 0.174 | 0.461 | 0.516 | 0.330 | 3172851 | 11488847 |
| hederagenin | 20 | 0.310 | 0.064 | −1.259 | −0.901 | 0.385 | 0.945 | −0.654 | −0.630 | −0.540 | 3172852 | 11489437 |
| sapindoside A | 20 | −3.886 | −4.232 | −4.558 | −3.760 | −2.825 | −4.058 | −1.176 | −1.786 | 0.322 | 3172853 | 11489438 |
| lycorine | 20 | −2.922 | 0.198 | −4.612 | −2.570 | −2.639 | −1.288 | −1.119 | 1.103 | −5.387 | 3172856 | 11488234 |
| cytisine | 20 | −0.076 | 0.069 | 0.571 | −0.397 | 1.150 | 0.186 | 0.025 | 0.027 | 0.052 | 3172862 | 11488286 |
| cyclosporine | 20 | 0.161 | 0.951 | −0.887 | −0.126 | 0.791 | −0.632 | −0.627 | −0.631 | −0.496 | 3172968 | 11489300 |
| azadirachtin | 20 | −0.479 | 0.299 | −1.018 | −0.670 | 0.494 | −0.421 | 0.119 | 0.152 | 0.042 | 3172973 | 11489402 |
| ouabain | 20 | −0.631 | 0.320 | −2.101 | −1.093 | 0.466 | 0.262 | −1.278 | −0.986 | −1.547 | 3172974 | 11488900 |
| diosgenin | 20 | −0.044 | −0.057 | −1.597 | −0.353 | 0.048 | −0.679 | −0.245 | −0.445 | 0.146 | 3172979 | 11488034 |
| pristimerin | 20 | −5.473 | −8.234 | −6.164 | −3.651 | −3.693 | −5.896 | ND | ND | ND | 3172984 | 11488362 |
| hetacillin | 20 | −0.642 | 1.231 | −0.663 | −0.787 | 1.132 | 0.731 | −0.200 | −0.094 | −0.302 | 3173079 | 11488932 |
| metoprolol | 9.58 | 0.437 | 0.449 | −1.894 | −0.997 | 0.253 | 0.496 | −0.423 | −0.430 | −0.342 | 3173081 | 11467881 |
| metoprolol | 20 | 0.326 | 1.130 | −1.660 | −0.515 | 0.652 | 0.596 | 0.450 | 0.546 | 0.241 | 3173081 | 11488873 |
| spiramycin | 20 | 0.176 | 0.288 | −0.770 | −0.686 | 0.379 | 0.216 | −0.691 | −0.867 | −0.204 | 3173083 | 11489377 |
| neomycin | 20 | −1.255 | −1.028 | −1.478 | −0.599 | 1.203 | 0.734 | 0.817 | 0.598 | 1.160 | 3173091 | 11488285 |
| dimenhydrinate | 20 | −1.060 | −0.597 | −1.505 | −0.890 | 0.339 | 0.251 | −0.225 | −0.086 | −0.390 | 3173092 | 11488808 |
| leoidin | 20 | 0.035 | −0.082 | 0.994 | −1.352 | 1.481 | 0.341 | −0.611 | −0.523 | −0.638 | 3173106 | 11488437 |
| tomatidine | 20 | 0.490 | 0.445 | −1.376 | −0.425 | 1.323 | 0.913 | −1.375 | −1.473 | −0.857 | 3173115 | 11488248 |
| ceftriaxone | 20 | −0.792 | −0.735 | −1.040 | −0.461 | −0.002 | 0.481 | −0.433 | −0.439 | −0.382 | 3173210 | 11487838 |
| puromycin | 20 | −5.718 | −7.962 | −5.624 | −3.989 | −2.665 | −5.573 | −3.348 | −4.017 | −1.331 | 3173213 | 11488712 |
| oxacillin sodium | 20 | −1.101 | −0.038 | −1.064 | −0.432 | 0.572 | −0.361 | −0.790 | −0.629 | −0.881 | 3173215 | 11488844 |
| aconitine | 20 | 0.132 | 0.350 | −0.584 | −0.073 | 1.038 | 0.751 | 0.169 | 0.356 | −0.198 | 3173217 | 11488453 |
| 3-methylxanthine | 20 | 0.036 | 0.704 | −0.859 | −0.149 | 0.472 | 0.692 | −1.241 | −1.286 | −0.856 | 3173234 | 11488318 |
| pinacidil | 16.3 | −0.477 | 0.891 | −0.889 | −0.614 | 0.383 | 0.027 | −0.146 | 0.038 | −0.498 | 3173239 | 11467394 |
| pinacidil | 20 | −0.062 | −0.566 | −0.142 | −0.949 | 0.655 | 1.030 | −0.515 | −0.301 | −0.814 | 3173239 | 11489555 |
| androsterone | 20 | −0.882 | 0.711 | −0.685 | −0.846 | 0.619 | −0.302 | −0.061 | −0.099 | 0.009 | 3173241 | 11488748 |
| zoxazolamine | 23.72 | −0.415 | 1.480 | −0.303 | −0.068 | 1.630 | 0.552 | 0.447 | 0.517 | 0.203 | 3173242 | 11467476 |
| zoxazolamine | 20 | −0.121 | −0.099 | −0.667 | −0.780 | 0.657 | 1.131 | −0.531 | −0.242 | −1.036 | 3173242 | 11488587 |
| cefuroxime | 20 | −0.846 | 0.476 | −0.536 | −0.358 | 0.366 | −0.495 | 0.816 | 0.790 | 0.699 | 3173342 | 11488522 |
| lasalocid | 20 | −1.090 | −0.906 | −1.771 | −1.178 | −2.993 | −1.690 | −1.524 | 0.274 | −4.908 | 3173346 | 11488680 |
| deoxygedunin | 20 | −0.326 | 1.094 | −0.532 | 1.327 | −0.640 | 1.083 | −1.229 | −1.298 | −0.808 | 3173352 | 11488148 |
| betulinic acid | 20 | 0.850 | −0.271 | −2.381 | −1.220 | 0.202 | 0.481 | 0.145 | 0.069 | 0.259 | 3173357 | 11488632 |
| ursocholanic acid | 20 | 0.050 | 0.349 | −0.246 | −1.738 | 0.978 | 1.303 | −0.920 | −0.946 | −0.657 | 3173360 | 11488440 |
| tomatine | 20 | −4.793 | −5.117 | −4.122 | −2.423 | −2.084 | −3.979 | −2.151 | −2.158 | −1.725 | 3173364 | 11488756 |
| lunarine | 20 | −0.579 | 0.517 | −0.938 | −0.795 | 0.385 | 0.685 | −0.945 | −0.880 | −0.856 | 3173368 | 11488159 |
| totarol acetate | 20 | −0.273 | 0.031 | 0.730 | −0.556 | 0.452 | 1.207 | 0.574 | 0.830 | −0.125 | 3173369 | 11488083 |
| isoxsuprine | 20 | −0.141 | −0.160 | −1.776 | −1.246 | 1.375 | 0.295 | −0.050 | −0.267 | 0.463 | 3173462 | 11488881 |
| nitrofurantoin | 16.8 | 0.779 | −0.264 | −0.586 | 0.358 | −0.261 | 0.122 | −0.224 | −0.031 | −0.618 | 3173466 | 11467316 |
| nitrofurantoin | 20 | 0.233 | 0.261 | −1.489 | 0.956 | 0.728 | 1.050 | 0.224 | 0.250 | 0.202 | 3173466 | 11488862 |
| quinidine | 20 | 0.916 | 0.901 | −0.233 | 1.187 | 1.326 | 1.286 | 0.849 | 0.956 | 0.501 | 3173468 | 11488224 |
| estradiol | 20 | 0.400 | 0.137 | −1.040 | −0.085 | 0.310 | 0.389 | 0.290 | 0.410 | −0.058 | 3173471 | 11487879 |
| troleandomycin | 20 | −1.520 | 0.194 | −1.250 | −0.780 | 0.099 | −0.063 | −1.234 | −1.277 | −0.905 | 3173475 | 11489301 |
| friedelin | 20 | 0.082 | 0.462 | −1.362 | −0.867 | 0.449 | 0.614 | −1.043 | −1.150 | −0.600 | 3173478 | 11488168 |
| sennoside A | 20 | 0.602 | −0.358 | −0.769 | −0.640 | 0.137 | 0.362 | −1.374 | −1.093 | −1.628 | 3173485 | 11489458 |
| colchiceine | 20 | −0.333 | −0.848 | −3.186 | −0.812 | 0.469 | −0.908 | 1.832 | 1.935 | 1.192 | 3173492 | 11488112 |
| formestane | 20 | −0.582 | −0.830 | −0.725 | −0.608 | 1.013 | −0.115 | 0.097 | 0.050 | 0.121 | 3173493 | 11487845 |
| pyrantel pamoate | 20 | −1.934 | −0.426 | −0.189 | −2.829 | 0.592 | 0.640 | −0.468 | −0.500 | −0.327 | 3173593 | 11489120 |
| nystatin | 20 | 0.794 | 0.843 | 0.161 | −0.356 | −0.043 | −0.059 | 0.326 | 0.284 | 0.289 | 3173595 | 11487887 |
| naloxone | 20 | 0.399 | 2.752 | −0.667 | 0.353 | 0.782 | 0.362 | 0.316 | 0.420 | 0.107 | 3173600 | 11488865 |
| gitoxigenin diacetate | 20 | 0.187 | 1.895 | −1.077 | −0.792 | 0.508 | 0.684 | −0.594 | −0.649 | −0.322 | 3173612 | 11488177 |
| beta-sitosterol | 20 | −0.768 | −0.419 | −1.974 | −0.679 | 0.049 | 0.061 | 0.098 | 0.046 | 0.218 | 3173615 | 11488194 |
| deoxyguanosine | 20 | −0.819 | 0.734 | −0.703 | −0.268 | −0.110 | 0.695 | −0.001 | −0.090 | 0.245 | 3173625 | 11488960 |
| ergosterol acetate | 20 | 0.023 | 1.578 | −1.580 | 0.050 | 0.270 | 0.346 | −0.380 | −0.220 | −0.680 | 3173626 | 11489745 |
| pempidine | 13.1 | −1.023 | 0.278 | −2.128 | −0.967 | 1.119 | 0.163 | −0.656 | −0.425 | −0.998 | 3173628 | 11467831 |
| pempidine | 20 | −0.822 | 0.192 | −1.419 | −0.376 | 0.861 | 0.146 | −0.874 | −1.035 | −0.378 | 3173628 | 11489371 |
| cephalexin | 20 | 0.343 | 0.201 | −0.506 | −0.353 | −0.316 | 0.678 | −0.957 | −0.950 | −0.783 | 3173633 | 11489277 |
| amikacin | 20 | 0.034 | −0.527 | −1.688 | −0.116 | 0.301 | −0.090 | 0.332 | 0.268 | 0.338 | 3173722 | 11487918 |
| piperacillin | 20 | −0.990 | 0.634 | −1.188 | −0.150 | 0.645 | 0.544 | −0.548 | −0.339 | −0.857 | 3173725 | 11489102 |
| pasiniazid | 20 | −0.883 | 1.328 | −0.807 | −0.809 | 0.502 | 0.590 | −0.059 | −0.022 | −0.164 | 3173726 | 11489752 |
| dihydrorotenone | 20 | 0.390 | −5.350 | −4.020 | −0.314 | −0.682 | −3.153 | −2.179 | −2.212 | −1.732 | 3173749 | 11487997 |
| cortisone | 20 | 0.644 | 0.319 | −0.146 | −0.232 | 0.527 | −0.078 | −0.428 | −0.165 | −0.830 | 3173755 | 11489640 |
| etoposide | 20 | −1.541 | 0.749 | −2.352 | 0.050 | −1.736 | −0.491 | 0.725 | 0.355 | 1.373 | 3173759 | 11488278 |
| berbamine | 20 | −1.126 | 0.168 | −1.021 | −0.289 | 0.153 | 0.445 | −0.345 | −0.166 | −0.636 | 3173760 | 11489211 |
| cefaclor | 10.88 | 0.762 | 0.678 | −1.329 | −0.759 | 0.387 | −0.764 | 0.331 | 0.535 | −0.161 | 3173761 | 11467633 |
| loracarbef | 10.88 | 0.317 | −0.098 | −0.158 | −0.229 | −0.236 | 0.622 | 0.129 | 0.215 | −0.088 | 3173761 | 11468250 |
| cefaclor | 20 | −0.091 | 0.137 | −0.752 | −0.772 | 0.297 | 0.231 | −0.547 | −0.506 | −0.435 | 3173761 | 11489005 |
| amphotericin B | 20 | −0.453 | 0.979 | −1.466 | −0.292 | 0.428 | −0.174 | −0.423 | −0.355 | −0.499 | 3173762 | 11488634 |
| chlorogenic acid | 11.28 | −0.739 | 0.607 | −0.944 | −0.943 | 0.188 | 0.039 | 0.096 | 0.348 | −0.436 | 3173763 | 11467575 |
| chlorogenic acid | 20 | −0.720 | 0.442 | −1.629 | −0.960 | −0.173 | 0.279 | −0.263 | −0.374 | 0.044 | 3173763 | 11489714 |
| neohesperidin dihydrochalcone | 20 | 0.061 | 2.411 | −0.964 | 0.231 | 0.934 | 0.454 | −0.190 | −0.372 | 0.259 | 3173852 | 11488144 |
| roxithromycin | 20 | 0.513 | 0.595 | −1.163 | −0.352 | 1.120 | 0.218 | −1.024 | −0.962 | −0.958 | 3173855 | 11489367 |
| atractyloside | 5.5 | −0.186 | 1.229 | −0.683 | −0.663 | 1.160 | 0.174 | −0.311 | −0.117 | −0.644 | 3173859 | 11467885 |
| atractyloside | 20 | −1.047 | −0.662 | −1.079 | −1.061 | 1.313 | 0.233 | 0.161 | 0.350 | −0.182 | 3173859 | 11488914 |
| nalbuphine | 20 | −1.422 | 0.390 | −1.511 | −0.919 | 0.042 | 0.276 | −0.210 | −0.124 | −0.342 | 3173860 | 11489219 |
| mexicanolide | 20 | 0.816 | 1.008 | 0.311 | −0.046 | 0.479 | −0.406 | 0.457 | 0.204 | 0.819 | 3173867 | 11488056 |
| sericetin diacetate | 20 | 0.051 | 0.104 | −1.876 | 2.433 | 0.923 | 0.565 | −0.479 | −0.257 | −0.892 | 3173879 | 11487994 |
| bacampicillin | 20 | 1.335 | 1.827 | −0.488 | 0.062 | −0.192 | 0.127 | −1.269 | −1.161 | −1.241 | 3173981 | 11489339 |
| gitoxigenin | 20 | −0.731 | 1.363 | −1.879 | −0.540 | 1.427 | 0.100 | −0.281 | −0.465 | 0.081 | 3173991 | 11488104 |
| totarol | 20 | −0.254 | −0.267 | 2.622 | −0.623 | 0.932 | 0.561 | −0.724 | −0.384 | −1.324 | 3173992 | 11488087 |
| yohimbinic acid | 11.76 | −0.378 | −0.041 | −0.978 | −0.282 | 0.723 | 0.509 | 0.280 | 0.670 | −0.554 | 3173993 | 11467738 |
| yohimbic acid | 20 | −0.310 | 0.412 | −1.098 | −0.825 | 0.706 | −0.364 | −0.564 | −0.534 | −0.479 | 3173993 | 11488267 |
| digitonin | 20 | −2.207 | −1.486 | −3.750 | 0.139 | −0.404 | −1.798 | 0.135 | 0.392 | −0.476 | 3173995 | 11488094 |
| andirobin | 20 | 0.649 | 2.119 | −0.285 | −0.261 | 0.082 | 0.496 | −0.225 | −0.230 | −0.218 | 3173997 | 11488040 |
| grayanotoxin I | 20 | 0.289 | −0.096 | 0.059 | −0.414 | −0.171 | 0.127 | 0.729 | 0.652 | 0.807 | 3173998 | 11488373 |
| hecogenin acetate | 20 | −0.683 | 0.317 | −1.994 | −0.849 | 1.816 | −0.050 | −0.434 | −0.610 | −0.030 | 3173999 | 11488092 |
| smilagenin | 20 | −0.808 | 0.483 | −0.266 | −1.153 | 0.310 | 0.178 | 0.310 | 0.289 | 0.354 | 3174000 | 11488193 |
| pararosaniline | 20 | −1.866 | −6.271 | −4.910 | −3.740 | −2.532 | −2.574 | −3.501 | −2.247 | −5.433 | 3174099 | 11487823 |
| mebhydrolin | 7.08 | −0.133 | −0.958 | −1.229 | −0.707 | 0.498 | −0.530 | −0.637 | −0.506 | −0.778 | 3174101 | 11467603 |
| mebhydrolin | 20 | −1.749 | −0.854 | −0.416 | −0.755 | 0.225 | −0.059 | −1.338 | −1.268 | −1.242 | 3174101 | 11488496 |
| hydroxyzine | 20 | −0.625 | 0.258 | −0.494 | −0.800 | 1.424 | −0.524 | −0.625 | −0.468 | −0.748 | 3174102 | 11488816 |
| parthenolide | 20 | −5.215 | −7.573 | −6.275 | −3.817 | −0.693 | −4.905 | −4.012 | −4.775 | −1.601 | 3174103 | 11489036 |
| pyrvinium pamoate | 20 | −2.225 | −5.052 | −4.560 | −2.938 | −1.987 | −1.309 | −3.051 | −2.082 | −4.397 | 3174105 | 11489123 |
| amiprilose | 20 | 0.222 | 0.110 | 0.819 | 0.027 | 0.174 | −0.501 | 0.441 | 0.317 | 0.622 | 3174106 | 11489335 |
| sisomicin | 20 | −1.118 | 0.313 | −1.365 | −0.820 | 0.032 | 0.158 | −0.360 | −0.407 | −0.193 | 3174107 | 11489130 |
| fucostanol | 20 | −1.432 | −0.453 | −3.092 | −0.990 | −0.122 | 0.205 | −0.411 | −0.385 | −0.339 | 3174115 | 11489450 |
| roccellic acid | 20 | 0.383 | 1.062 | −1.673 | −0.462 | 0.175 | 0.909 | −0.208 | −0.128 | −0.308 | 3174120 | 11488388 |
| nigericin | 20 | −1.744 | −3.165 | −3.329 | 1.324 | −3.323 | −2.497 | −2.367 | −1.736 | −3.122 | 3174220 | 11488991 |
| bretylium | 20 | −0.775 | −0.361 | −0.446 | −1.114 | 0.632 | 0.560 | 0.996 | 1.057 | 0.732 | 3174226 | 11488289 |
| ajmaline | 20 | −0.003 | 0.454 | 0.064 | −0.147 | 1.176 | 0.191 | 0.979 | 0.728 | 1.229 | 3174227 | 11487896 |
| dihydrostreptomycin | 20 | 0.042 | 0.519 | −0.944 | −0.650 | 0.203 | 0.491 | −0.421 | −0.329 | −0.454 | 3174228 | 11488818 |
| theaflavin | 20 | −0.618 | 0.975 | −0.396 | −0.825 | −0.159 | 0.599 | −0.373 | −0.328 | −0.366 | 3174236 | 11489581 |
| arbutin | 20 | −0.752 | 0.483 | −0.695 | −0.536 | 0.188 | 0.067 | 0.044 | 0.163 | −0.219 | 3174242 | 11488478 |
| leucopterin | 20 | −0.307 | −0.202 | −2.409 | −0.179 | 1.106 | 0.494 | 0.752 | 0.995 | 0.150 | 3174244 | 11488403 |
| phloridzin | 20 | 0.317 | 0.210 | −0.331 | −0.473 | −0.176 | 0.286 | 0.052 | −0.036 | 0.209 | 3174245 | 11488517 |
| deoxycholic acid | 20 | −0.892 | 0.037 | −1.242 | −1.081 | 0.761 | 0.185 | −0.302 | −0.333 | −0.139 | 3174249 | 11488172 |
| meclocycline | 5.76 | −0.275 | 0.410 | −1.589 | −1.670 | −0.005 | −0.222 | −0.126 | −0.412 | 0.481 | 3174345 | 11467604 |
| meclocycline | 20 | −0.581 | 0.172 | −1.677 | −1.519 | 0.162 | 0.012 | 0.381 | −0.142 | 1.368 | 3174345 | 11489221 |
| smilagenin acetate | 20 | −0.607 | −0.117 | −1.356 | −0.152 | 0.585 | 0.611 | −1.252 | −1.258 | −1.047 | 3174366 | 11488089 |
| carnosine | 20 | −0.898 | −0.025 | −1.714 | −1.120 | 0.886 | 0.073 | −0.626 | −0.572 | −0.578 | 3174367 | 11488270 |
| gitoxin | 20 | 0.002 | 1.035 | −0.671 | −0.627 | 1.680 | 0.826 | 0.380 | 0.391 | 0.277 | 3174369 | 11489192 |
| vancomycin | 20 | −0.443 | −0.409 | −0.759 | −0.160 | 1.370 | 0.092 | 0.176 | −0.291 | 1.039 | 3174477 | 11487885 |
| adrenaline | 20 | −0.736 | −0.317 | −1.299 | −1.202 | 0.190 | 0.173 | 0.000 | 0.076 | −0.076 | 3174481 | 11488809 |
| epiandrosterone | 20 | −0.177 | −0.525 | 1.264 | 0.291 | 0.791 | 0.256 | −0.340 | −0.569 | 0.240 | 3174484 | 11489724 |
| scopoline | 20 | 0.047 | 0.688 | −0.511 | −0.807 | 0.233 | 0.179 | −1.331 | −0.982 | −1.815 | 3174492 | 11488498 |
| glucosaminic acid | 20 | −0.238 | −0.832 | 0.276 | −0.814 | 0.607 | −0.516 | 0.053 | 0.080 | 0.030 | 3174493 | 11489726 |
| hypoxanthine | 20 | −0.202 | −0.570 | −0.965 | −2.156 | 1.178 | 0.608 | −1.781 | 0.575 | −6.147 | 3174602 | 11489723 |
| quebrachitol | 20 | −1.126 | −0.067 | −1.017 | −0.561 | 0.361 | −0.256 | −0.508 | −0.525 | −0.402 | 3174608 | 11489804 |
| atorvastatin | 20 | 0.043 | −0.945 | −1.529 | −0.744 | −0.940 | −1.023 | 0.764 | 0.618 | 0.924 | 3174609 | 11489401 |
| veratridine | 20 | 0.161 | 0.642 | −0.084 | −0.453 | 0.719 | 0.294 | 0.021 | 0.203 | −0.352 | 3174610 | 11489397 |
| cholest-5-en-3-one | 20 | 0.207 | 1.026 | −0.937 | −0.686 | 0.629 | 0.600 | 0.959 | 0.943 | 0.850 | 3174615 | 11488452 |
| D-cycloserine | 39.18 | −0.586 | 0.002 | −0.453 | −0.166 | 0.042 | −0.620 | 0.242 | 0.146 | 0.384 | 3176921 | 11468234 |
| cortisone | 11.1 | −0.705 | 0.407 | −1.467 | −0.740 | 0.184 | 0.063 | 0.059 | 0.177 | −0.184 | 3176928 | 11467421 |
| cefsulodin | 7.5 | 0.117 | 0.384 | −2.002 | −0.652 | 0.951 | 0.836 | −0.492 | −0.377 | −0.637 | 3176931 | 11467962 |
| 1-benzyloxycarbonylaminophenethyl | 20 | −5.468 | −8.086 | −6.339 | −4.136 | −4.285 | −5.377 | −3.760 | −4.030 | −2.480 | 3176939 | 11489309 |
| fluvoxamine | 12.56 | −0.521 | −0.252 | −0.012 | −0.258 | 0.826 | 0.483 | 0.445 | 0.590 | 0.052 | 3177109 | 11468143 |
| famotidine | 11.86 | −0.118 | 1.643 | −1.496 | −0.409 | −0.094 | 0.528 | −0.974 | −0.963 | −0.847 | 3177110 | 11467252 |
| ifenprodil | 8.42 | 0.882 | 1.184 | 0.999 | 0.021 | 2.016 | 0.674 | −0.614 | −0.356 | −1.023 | 3177204 | 11467459 |
| metergoline | 9.92 | 0.243 | −1.286 | −2.643 | 0.173 | 0.787 | −1.385 | −0.572 | −0.652 | −0.294 | 3177235 | 11467512 |
| betamethasone | 20 | 0.284 | −0.954 | −0.977 | 0.629 | 0.940 | −0.109 | 1.063 | 0.780 | 1.370 | 3177311 | 11487916 |
| lathosterol | 20 | 0.543 | 0.872 | 0.907 | −0.333 | 0.526 | 0.513 | −0.655 | −0.691 | −0.410 | 3177312 | 11488390 |
| garcinolic acid | 20 | −1.005 | 0.053 | −1.674 | −2.471 | 0.651 | −0.290 | −0.570 | −0.474 | −0.615 | 3177314 | 11488423 |
| quercitrin | 20 | 0.140 | 1.903 | −0.628 | −1.249 | −0.769 | 0.555 | −0.402 | −0.644 | 0.216 | 3177315 | 11488145 |
| convallatoxin | 20 | −0.832 | 0.676 | −1.168 | −0.507 | 0.948 | −0.282 | −0.371 | −0.285 | −0.392 | 3177316 | 11489055 |
| hydroxyprogesterone | 20 | 0.895 | 0.883 | −0.258 | −0.099 | 0.872 | 0.560 | −0.869 | −0.820 | −0.726 | 3177322 | 11489035 |
| tetrahydrocortisone | 20 | −0.055 | −0.693 | −0.738 | −0.571 | 0.385 | 0.107 | −0.663 | −0.806 | −0.204 | 3177324 | 11489641 |
| pancuronium | 6.98 | −1.055 | −0.433 | −1.235 | −0.576 | −0.179 | −0.073 | −0.453 | −0.374 | −0.543 | 3177327 | 11468182 |
| alfaxalone | 12.04 | 0.513 | −0.004 | −0.849 | −0.649 | 0.564 | 0.093 | 0.542 | 0.635 | 0.238 | 3177333 | 11468150 |
| larixol acetate | 20 | 0.353 | 0.412 | 0.101 | −0.432 | 1.144 | 0.494 | 0.325 | 0.062 | 0.728 | 3177379 | 11488134 |
| 4′-hydroxychalcone | 20 | −0.880 | 0.046 | −0.477 | 0.065 | 0.829 | 0.147 | −0.950 | −0.964 | −0.776 | 3177381 | 11488019 |
| fluocinolone | 20 | −0.508 | 0.341 | −1.445 | 1.685 | 0.067 | −0.050 | −0.823 | −0.898 | −0.456 | 3177385 | 11488780 |
| kanamycin A | 20 | 0.182 | 0.186 | −1.330 | −0.482 | 1.129 | 0.162 | 0.144 | 0.404 | −0.331 | 3177386 | 11488875 |
| noscapine | 20 | −0.689 | 0.824 | −0.648 | −0.510 | 0.430 | −0.297 | 0.703 | 0.700 | 0.635 | 3177388 | 11488803 |
| tobramycin | 20 | 0.089 | 0.932 | −1.891 | −0.443 | 0.298 | −0.107 | 1.081 | 0.956 | 1.057 | 3177389 | 11487894 |
| quinidine | 12.32 | −0.793 | −0.292 | −0.681 | −0.382 | 0.645 | −1.130 | 0.061 | 0.239 | −0.305 | 3177396 | 11467428 |
| fludrocortisone acetate | 20 | −0.750 | 0.458 | −1.485 | −0.196 | −0.743 | 0.274 | −0.475 | −0.511 | −0.251 | 3177451 | 11488790 |
| fluocinonide | 20 | −0.905 | 0.097 | −0.828 | −0.144 | 0.316 | −0.368 | 0.073 | 0.220 | −0.165 | 3177453 | 11488851 |
| cholesterol | 20 | −0.465 | 1.823 | −1.608 | −0.480 | 0.973 | −0.060 | −1.174 | −1.232 | −0.783 | 3177456 | 11488400 |
| dehydrocholic acid | 20 | 0.134 | 0.643 | −1.560 | −0.858 | 0.931 | 0.257 | 0.457 | 0.463 | 0.346 | 3177457 | 11489191 |
| estrone acetate | 20 | 0.145 | 0.679 | −0.536 | −0.663 | 1.773 | 0.398 | 0.491 | 0.251 | 0.892 | 3177458 | 11489254 |
| anisodamine | 20 | −0.095 | 0.462 | −0.373 | −0.288 | 0.202 | 0.466 | −0.130 | −0.214 | 0.050 | 3177461 | 11488548 |
| betamethasone | 20 | −0.467 | −0.131 | −0.718 | −0.100 | 0.346 | −0.942 | −0.113 | −0.292 | 0.341 | 3177462 | 11489076 |
| cephradine | 20 | −0.745 | −0.188 | −2.067 | −0.807 | 0.177 | 0.431 | −0.562 | −0.484 | −0.539 | 3177463 | 11489050 |
| capreomycin | 20 | 0.882 | −0.979 | 0.181 | −0.639 | −0.197 | 0.523 | −0.163 | −0.284 | 0.071 | 3177464 | 11487877 |
| oleandrin | 20 | 0.691 | 1.192 | −0.702 | −1.297 | 0.501 | 0.175 | −0.262 | 0.148 | −0.979 | 3177465 | 11488989 |
| paromomycin | 20 | −0.604 | 0.853 | −0.913 | −0.507 | 0.620 | −1.032 | 0.573 | 0.546 | 0.508 | 3177517 | 11488675 |
| picropodophyllotoxin acetate | 20 | 0.072 | 1.141 | −2.400 | −0.390 | −1.714 | −0.889 | 0.731 | 0.716 | 0.601 | 3177521 | 11488623 |
| pyrromycin | 20 | −3.145 | −2.578 | −4.056 | −3.412 | −2.008 | −1.138 | −3.027 | −2.886 | −2.670 | 3177523 | 11488510 |
| nateglinide | 20 | −0.543 | 0.143 | −1.183 | 0.146 | −0.581 | −0.517 | −0.828 | −0.933 | −0.420 | 3177524 | 11489490 |
| ornithine alphaketoglutarate | 20 | −1.493 | 0.259 | −2.104 | −0.917 | 0.840 | 0.085 | −0.715 | −0.605 | −0.727 | 3177525 | 11489060 |
| podophyllotoxin acetate | 20 | −0.983 | −3.008 | −3.102 | 1.238 | −1.656 | −0.777 | 1.407 | 1.426 | 1.125 | 3177591 | 11489497 |
| vancomycin | 2.76 | 0.012 | 0.458 | −0.636 | −0.833 | 0.921 | 0.898 | −0.289 | −0.139 | −0.538 | 3177604 | 11467645 |
| seneciphylline | 12 | −0.903 | −0.239 | −0.529 | −0.146 | 0.686 | −0.683 | 0.156 | 0.117 | 0.207 | 3179848 | 11467747 |
| cephaeline | 8.58 | −5.685 | −7.376 | −6.133 | −3.335 | −2.842 | −5.599 | 0.138 | 0.347 | −0.334 | 3180147 | 11467576 |
| hydrastine | 10.44 | 0.263 | 0.516 | −0.863 | −0.212 | −0.017 | 0.525 | 0.063 | 0.032 | 0.113 | 3180327 | 11467729 |
| conessine | 11.22 | 0.176 | −0.350 | −1.669 | −0.471 | 1.082 | −1.296 | 0.380 | 0.265 | 0.533 | 3187609 | 11467786 |
| protoveratrine A | 5.04 | 0.433 | −0.715 | 0.464 | −0.287 | 1.528 | −0.449 | 0.253 | 0.083 | 0.556 | 3187610 | 11467787 |
| sulmazole | 13.92 | −0.386 | 1.451 | −0.531 | 1.306 | 0.713 | 1.129 | −0.572 | −0.514 | −0.582 | 3187611 | 11467789 |
| flunisolide | 9.2 | −0.594 | 2.843 | −0.529 | 1.025 | 0.560 | 0.715 | −0.106 | −0.123 | −0.076 | 3187612 | 11467791 |
| helveticoside | 7.48 | −0.288 | 0.775 | −1.371 | 0.073 | 0.744 | 0.833 | 0.266 | 0.515 | −0.297 | 3187613 | 11467794 |
| butirosin | 7.2 | 0.351 | 1.806 | −0.874 | 0.889 | 0.651 | 0.589 | −0.043 | −0.077 | 0.033 | 3187614 | 11467798 |
| picrotoxinin | 13.68 | 0.091 | 0.839 | −1.834 | −0.355 | 0.724 | 0.990 | −0.116 | 0.095 | −0.523 | 3187615 | 11467800 |
| benfotiamine | 8.58 | −0.050 | 0.589 | −1.356 | −0.899 | 0.881 | 0.432 | −0.230 | −0.075 | −0.505 | 3187616 | 11467802 |
| lanatoside C | 3.94 | 0.288 | 0.484 | −1.387 | −1.272 | 1.913 | 0.987 | −0.227 | −0.136 | −0.358 | 3187617 | 11467804 |
| avermectin B1 | 4.58 | 1.166 | 0.659 | −0.348 | −0.149 | 1.616 | 0.671 | −0.150 | −0.164 | −0.090 | 3187618 | 11467808 |
| solasodine | 9.68 | 0.047 | 0.668 | −1.453 | −0.916 | 1.169 | 0.552 | −0.666 | −0.709 | −0.453 | 3187619 | 11467811 |
| cis-nanophine | 35.34 | −0.528 | −0.212 | −1.551 | −0.963 | 1.222 | 0.531 | −0.115 | −0.009 | −0.302 | 3187620 | 11467814 |
| deltaline | 7.88 | −0.669 | −0.108 | −2.022 | −0.929 | 1.524 | 0.294 | 0.098 | 0.107 | 0.064 | 3187622 | 11467821 |
| beta-escin | 3.54 | −4.210 | −2.955 | −4.299 | −2.682 | −1.103 | −3.298 | −0.967 | −0.781 | −1.155 | 3187623 | 11467824 |
| fluorocurarine | 13.02 | −0.665 | 0.010 | −0.478 | 0.130 | 0.525 | 0.243 | −0.014 | 0.119 | −0.272 | 3187624 | 11467828 |
| beta-belladonnine | 6.66 | −0.094 | −0.245 | −2.121 | −0.640 | 1.001 | 0.178 | −0.451 | −0.292 | −0.668 | 3187625 | 11467830 |
| karakoline | 10.6 | −0.568 | −0.087 | −1.657 | −1.112 | 1.495 | 0.002 | −0.263 | −0.087 | −0.569 | 3187744 | 11467835 |
| estropipate | 11.42 | −0.518 | −0.300 | −0.645 | −1.201 | 1.736 | 0.041 | −0.181 | −0.270 | 0.039 | 3187745 | 11467836 |
| napelline | 11.12 | −0.454 | 0.567 | −0.919 | −0.860 | 0.692 | 0.185 | 0.004 | 0.128 | −0.243 | 3187746 | 11467838 |
| fillalbin | 13.72 | −0.600 | −0.022 | −1.166 | −0.608 | 1.228 | 0.700 | −0.441 | −0.163 | −0.916 | 3187747 | 11467839 |
| tadjakonine | 7.5 | −0.403 | −0.136 | −2.254 | −0.450 | 1.190 | −0.096 | 0.172 | 0.187 | 0.092 | 3187748 | 11467840 |
| cefuroxime | 9.42 | −0.256 | 0.739 | 0.036 | −0.292 | 0.761 | 0.579 | −0.317 | −0.397 | −0.088 | 3187749 | 11467868 |
| ergocryptine-alpha | 6.94 | 2.729 | 1.621 | −1.580 | −0.856 | 0.377 | 1.295 | 0.143 | 0.382 | −0.378 | 3187750 | 11467875 |
| streptozosin | 15.08 | −0.478 | 0.524 | −1.635 | −0.996 | 0.393 | 0.701 | −0.256 | −0.052 | −0.636 | 3187751 | 11467880 |
| flumethasone | 9.74 | −1.279 | −0.148 | −2.146 | −0.541 | −0.131 | 0.569 | −0.930 | −1.042 | −0.538 | 3187752 | 11467882 |
| medrysone | 11.62 | −0.104 | 0.397 | −1.658 | −0.181 | 0.293 | 0.345 | −0.018 | 0.091 | −0.247 | 3187753 | 11467891 |
| flunixin | 8.14 | −0.127 | 0.853 | −1.908 | −0.285 | 0.496 | 0.847 | −0.300 | −0.010 | −0.860 | 3187754 | 11467892 |
| spiramycin | 4.74 | 0.003 | 0.427 | −1.238 | −0.834 | 1.762 | 0.865 | −0.313 | −0.476 | 0.061 | 3187755 | 11467893 |
| monensin | 5.96 | −0.307 | −3.555 | −3.197 | 1.556 | −1.933 | −2.647 | −0.958 | 0.698 | −4.135 | 3187756 | 11467896 |
| ribostamycin | 8.8 | −0.815 | −0.466 | −1.657 | −0.217 | −0.168 | −0.664 | 0.029 | 0.313 | −0.552 | 3187757 | 11467906 |
| guanadrel | 18.76 | −0.871 | 0.589 | −0.889 | −0.629 | 2.273 | −0.062 | −0.912 | −0.898 | −0.766 | 3187758 | 11467915 |
| alclometasone dipropionate | 7.68 | −0.005 | −0.333 | −1.105 | −0.064 | −0.101 | −0.061 | 0.262 | 0.217 | 0.301 | 3187759 | 11467919 |
| fluocinonide | 8.08 | −0.747 | −0.851 | −2.438 | −0.408 | −0.931 | −0.135 | −0.641 | −0.453 | −0.899 | 3187760 | 11467922 |
| hexylcaine | 15.3 | −0.243 | −0.043 | −1.093 | −0.567 | 0.761 | −0.287 | −0.420 | −0.510 | −0.150 | 3187761 | 11467936 |
| eucatropine | 13.72 | 0.195 | −0.577 | −1.193 | −0.394 | 0.182 | 0.279 | −0.104 | −0.052 | −0.193 | 3187762 | 11467942 |
| bucladesine | 8.52 | 0.426 | 1.159 | −2.045 | −0.834 | 0.838 | 0.934 | −0.946 | −0.833 | −1.003 | 3187884 | 11467961 |
| lasalocid | 6.78 | −1.076 | −2.539 | −3.481 | −1.518 | −2.547 | −2.028 | −1.296 | 0.500 | −4.696 | 3187885 | 11467976 |
| novobiocin | 6.52 | −1.306 | −0.242 | −1.973 | −0.828 | 1.037 | −0.043 | −0.582 | −0.600 | −0.427 | 3187886 | 11467982 |
| iocetamic acid | 6.52 | −1.121 | −0.565 | −1.752 | −0.748 | 1.244 | −0.407 | −0.226 | −0.035 | −0.572 | 3187887 | 11467986 |
| securinine | 18.42 | −1.284 | 0.472 | −2.816 | 0.636 | −1.392 | 0.070 | −0.934 | −0.826 | −0.973 | 3187888 | 11467989 |
| nafcillin | 9.66 | −1.063 | −0.049 | −2.074 | −0.851 | 0.785 | −0.118 | −0.589 | −0.629 | −0.392 | 3187889 | 11467991 |
| doxycycline | 9 | −0.338 | −0.232 | −1.655 | −1.288 | 1.003 | −0.089 | −0.415 | −0.712 | 0.270 | 3187892 | 11468000 |
| roxithromycin | 4.78 | 0.271 | 0.042 | −2.130 | −0.519 | 0.795 | 0.424 | −0.703 | −0.378 | −1.216 | 3187893 | 11468002 |
| 5-azacytidine | 16.38 | −1.939 | −0.890 | −3.989 | −0.838 | −0.821 | −2.110 | 0.465 | 0.650 | −0.001 | 3187895 | 11468014 |
| paromomycin | 6.5 | −1.024 | −0.332 | −1.232 | −1.035 | 1.005 | 0.513 | −0.756 | −0.592 | −0.930 | 3187896 | 11468015 |
| digoxigenin | 10.24 | 0.352 | 2.192 | −0.157 | −0.237 | 0.038 | 0.884 | −0.418 | −0.283 | −0.628 | 3187897 | 11468031 |
| esculin | 11.76 | −0.845 | 0.709 | 0.034 | −0.526 | −0.253 | 0.588 | 0.048 | 0.031 | 0.077 | 3187898 | 11468040 |
| adrenosterone | 13.32 | −0.047 | 0.462 | 1.012 | −0.100 | 1.330 | 0.001 | −0.298 | −0.344 | −0.159 | 3187899 | 11468047 |
| ethynodiol diacetate | 10.4 | 0.251 | 1.264 | 0.680 | 0.072 | 1.234 | −1.332 | −1.258 | −1.144 | −1.264 | 3187900 | 11468056 |
| nizatidine | 12.06 | 0.923 | −0.251 | −0.226 | −0.390 | −0.285 | 0.876 | 0.172 | 0.122 | 0.223 | 3188016 | 11468069 |
| thioperamide | 13.68 | 0.134 | 0.955 | −0.938 | −0.530 | 0.031 | −0.025 | −0.481 | −0.535 | −0.290 | 3188017 | 11468070 |
| S(−)-terguride hydrogen | 11.74 | −1.081 | 0.209 | −0.697 | −0.459 | 0.735 | 0.125 | 1.039 | 0.914 | 1.095 | 3188018 | 11468075 |
| S(−)eticlopride | 11.74 | 0.195 | −0.365 | −1.030 | −0.105 | 0.367 | −0.169 | −0.007 | 0.070 | −0.164 | 3188019 | 11468080 |
| bephenium | 9 | −0.206 | −0.401 | −0.544 | −0.384 | 0.936 | −0.043 | 0.013 | −0.191 | 0.433 | 3188020 | 11468084 |
| tyloxapol | 4.02 | 0.048 | 0.309 | −0.123 | 0.014 | 0.665 | −0.039 | 0.169 | 0.067 | 0.332 | 3188022 | 11468102 |
| 6-hydroxytropinone | 25.78 | −0.058 | 2.418 | 0.313 | 0.515 | 0.507 | 0.933 | −0.176 | −0.044 | −0.423 | 3188025 | 11468115 |
| remoxipride | 10.78 | 0.412 | 1.197 | −0.924 | −0.502 | 0.342 | 0.778 | −0.300 | −0.090 | −0.700 | 3188026 | 11468119 |
| nitrocaramiphen | 11.96 | 0.636 | 1.010 | −0.403 | −0.360 | 0.003 | 0.296 | −0.486 | −0.317 | −0.748 | 3188027 | 11468129 |
| proscillaridin A | 7.54 | −0.350 | 0.760 | −0.058 | −0.665 | 1.100 | 0.573 | 0.136 | 0.127 | 0.122 | 3188028 | 11468134 |
| asiaticoside | 4.18 | −0.462 | −0.498 | −0.364 | 0.214 | 0.488 | 0.220 | 0.444 | 0.334 | 0.571 | 3188029 | 11468137 |
| ribavirin | 16.38 | −1.176 | −0.010 | −0.892 | −0.068 | 0.401 | −0.336 | 0.425 | 0.520 | 0.138 | 3188030 | 11468141 |
| lymecycline | 6.64 | −0.275 | −0.827 | 0.322 | 0.235 | −0.269 | −0.056 | 0.156 | 0.037 | 0.364 | 3188032 | 11468148 |
| meptazinol | 17.14 | −0.371 | −0.737 | −0.711 | −0.012 | 1.358 | −0.071 | −0.021 | 0.121 | −0.334 | 3188033 | 11468152 |
| apramycin | 7.42 | −0.376 | 0.061 | −0.744 | −0.663 | 0.886 | −0.064 | 0.414 | 0.193 | 0.781 | 3188034 | 11468153 |
| fursultiamine | 10.04 | −0.297 | 0.781 | 0.198 | −0.739 | 0.489 | 0.477 | 0.675 | 0.704 | 0.489 | 3188035 | 11468155 |
| pivampicillin | 8.62 | −0.907 | −0.731 | −0.242 | −0.195 | 0.452 | 0.326 | 0.215 | 0.111 | 0.372 | 3188145 | 11468157 |
| talampicillin | 8.3 | −0.644 | −0.313 | −0.118 | 0.120 | −0.299 | −0.064 | 0.676 | 0.417 | 1.074 | 3188146 | 11468158 |
| flucloxacillin | 8.82 | 0.149 | 0.368 | −0.995 | −0.072 | 0.823 | 0.272 | −0.387 | −0.177 | −0.755 | 3188147 | 11468159 |
| deptropine | 12 | 0.164 | −0.575 | −0.027 | −0.319 | 0.957 | −0.026 | 0.748 | 0.530 | 1.045 | 3188148 | 11468161 |
| tribenoside | 8.36 | 0.273 | −0.432 | 0.302 | 0.589 | 0.355 | 0.107 | −0.039 | −0.251 | 0.399 | 3188149 | 11468167 |
| rimexolone | 10.8 | −0.123 | −1.896 | 0.560 | 0.058 | −0.447 | −0.350 | −0.005 | −0.176 | 0.342 | 3188150 | 11468168 |
| nifurtimox | 13.92 | −0.554 | −1.006 | −0.144 | −0.499 | 0.669 | 0.004 | 0.156 | 0.144 | 0.127 | 3188151 | 11468172 |
| tocainide | 20.8 | −0.990 | −0.577 | −0.707 | −0.418 | 0.336 | 0.211 | 0.452 | 0.413 | 0.439 | 3188152 | 11468175 |
| benzathine benzylpenicillin | 6.78 | −0.568 | −0.853 | −0.151 | 0.729 | 0.478 | 0.277 | 0.624 | 0.429 | 0.889 | 3188153 | 11468176 |
| nomegestrol acetate | 10.8 | 0.011 | 1.167 | −0.220 | −0.331 | 1.945 | −0.425 | 1.104 | 1.008 | 1.082 | 3188154 | 11468181 |
| alcuronium chloride | 6.02 | −0.672 | 0.112 | −0.520 | −0.463 | 0.312 | 0.391 | −0.152 | −0.149 | −0.123 | 3188155 | 11468184 |
| pyrvinium pamoate | 5.18 | −2.444 | −4.764 | −3.107 | −2.252 | −1.211 | −0.619 | −2.809 | −1.358 | −5.233 | 3188156 | 11468188 |
| tridihexethyl | 12.56 | 0.631 | 1.450 | −0.048 | −0.566 | −1.685 | 0.959 | 0.063 | 0.309 | −0.465 | 3188157 | 11468190 |
| prednicarbate | 8.18 | −0.973 | 1.031 | −0.689 | −0.494 | −0.656 | 0.113 | −0.032 | −0.228 | 0.356 | 3188158 | 11468192 |
| repaglinide | 8.84 | 0.419 | 0.849 | −0.385 | −0.401 | −0.573 | 0.723 | −1.063 | −1.177 | −0.642 | 3188159 | 11468194 |
| piperacetazine | 9.74 | 1.287 | 0.544 | −0.961 | −0.104 | −0.798 | 0.357 | 0.480 | 0.543 | 0.253 | 3188160 | 11468196 |
| pivmecillinam | 9.1 | 0.383 | 0.785 | −0.509 | 2.087 | −0.149 | −0.192 | 0.206 | −0.058 | 0.683 | 3188161 | 11468201 |
| levopropoxyphene | 7.3 | −0.556 | 0.558 | −1.141 | −0.877 | 0.058 | 0.181 | −0.829 | −1.100 | −0.124 | 3188162 | 11468202 |
| phensuximide | 21.14 | 0.776 | 1.060 | 0.316 | 0.153 | −0.510 | 0.760 | −0.944 | −1.050 | −0.550 | 3188163 | 11468209 |
| thiethylperazine | 7.5 | 1.087 | 1.307 | 0.548 | 0.676 | 0.829 | −1.615 | −0.558 | −0.644 | −0.286 | 3188276 | 11468216 |
| cyproterone acetate | 9.6 | −0.234 | −0.262 | −0.638 | −0.339 | 0.871 | 0.055 | −0.301 | −0.342 | −0.160 | 3188277 | 11468222 |
| methiazole | 15.08 | −0.683 | −0.555 | −0.654 | 0.040 | −2.515 | −1.954 | 1.166 | 0.795 | 1.685 | 3188278 | 11468228 |
| condelphine | 8.9 | 0.090 | −0.472 | −0.983 | −0.122 | −0.040 | −0.031 | 0.500 | 0.530 | 0.325 | 3188279 | 11468231 |
| sulfadoxine | 12.88 | 0.265 | −0.471 | −1.186 | −0.176 | −0.172 | −0.293 | −0.275 | −0.323 | −0.148 | 3188280 | 11468242 |
| estriol | 13.88 | −0.462 | 0.043 | −0.799 | −0.415 | −0.143 | −0.136 | −0.169 | −0.210 | −0.052 | 3188281 | 11468244 |
| vitamin K2 | 8.96 | 0.095 | −0.620 | 0.693 | −0.246 | −0.635 | −0.948 | 0.517 | 0.427 | 0.587 | 3188282 | 11468248 |
| natamycin | 6 | −0.005 | −0.227 | −0.063 | −0.106 | 0.170 | −0.163 | 0.161 | 0.105 | 0.232 | 3188283 | 11468252 |
| verteporfin | 2.78 | 0.583 | −1.600 | −0.058 | −2.961 | −0.198 | −0.244 | 0.741 | 0.809 | 0.457 | 3188284 | 11468253 |
| rifabutin | 4.72 | −0.144 | −0.839 | 0.603 | 0.786 | 0.049 | −0.840 | 0.745 | 0.413 | 1.276 | 3188285 | 11468257 |
| viomycin | 5.84 | 0.648 | 0.473 | −0.224 | 0.073 | −0.798 | −0.234 | 1.247 | 1.176 | 1.142 | 3188286 | 11468261 |
| cefepime | 8.3 | 0.811 | −1.403 | −0.041 | 0.260 | −0.739 | −0.735 | 1.443 | 1.218 | 1.609 | 3188288 | 11468266 |
| clocortolone | 8.08 | 0.343 | −0.909 | 0.908 | 1.141 | −0.386 | −1.321 | 1.361 | 0.981 | 1.863 | 3188289 | 11468267 |
| benzonatate | 6.62 | −0.010 | 1.591 | −1.144 | −0.380 | 0.562 | 0.594 | −0.671 | −0.604 | −0.726 | 3188932 | 11467160 |
| norethynodrel | 13.4 | −0.683 | 0.040 | −1.593 | −0.453 | 0.988 | −0.145 | −0.575 | −0.567 | −0.521 | 3188934 | 11467172 |
| chloramphenicol | 12.38 | 0.041 | 0.012 | −1.351 | −1.241 | 0.363 | −0.276 | −0.415 | −0.617 | 0.035 | 3188935 | 11467179 |
| troleandomycin | 4.92 | −1.093 | 0.763 | −1.080 | −0.855 | 1.244 | −0.032 | 0.205 | 0.260 | 0.011 | 3188936 | 11467184 |
| amyleine | 17 | −0.948 | 0.509 | −0.719 | −0.888 | 1.284 | −0.010 | −0.235 | −0.182 | −0.344 | 3188937 | 11467197 |
| morantel | 10.8 | −1.011 | −0.170 | 0.027 | −0.644 | 1.553 | 0.004 | −0.640 | −0.520 | −0.810 | 3188938 | 11467209 |
| sulindac | 11.22 | 0.982 | 0.828 | −1.084 | −0.456 | 1.076 | −0.390 | 0.241 | 0.442 | −0.266 | 3188939 | 11467221 |
| ursolic acid | 8.76 | 1.135 | 2.069 | −0.501 | −0.433 | −0.570 | 0.667 | −0.218 | −0.106 | −0.440 | 3188940 | 11467237 |
| danazol | 11.86 | 0.059 | 1.289 | −0.696 | 0.440 | 0.542 | 0.651 | 0.205 | 0.123 | 0.281 | 3189048 | 11467253 |
| atropine-n-oxide | 13.1 | −0.171 | 0.490 | −0.526 | −0.805 | 0.320 | 0.395 | −0.205 | −0.157 | −0.305 | 3189049 | 11467255 |
| naltrexone | 11.72 | −0.597 | 0.242 | −1.150 | −0.694 | 0.452 | −0.098 | −0.521 | −0.758 | 0.025 | 3189051 | 11467264 |
| dehydrocholic acid | 9.56 | −0.337 | 0.386 | −1.619 | −0.202 | 0.099 | −0.074 | −1.516 | −1.635 | −1.014 | 3189053 | 11467271 |
| spironolactone | 9.6 | −0.906 | 0.240 | −0.480 | −0.546 | 0.249 | −0.651 | −0.105 | 0.044 | −0.411 | 3189054 | 11467276 |
| clindamycin | 9.42 | −0.784 | 0.714 | −1.753 | −0.367 | 0.094 | −0.521 | −1.034 | −1.119 | −0.693 | 3189055 | 11467285 |
| thioproperazine | 8.96 | 0.575 | −0.214 | −0.194 | −0.529 | 0.718 | −0.407 | −0.507 | −0.801 | 0.153 | 3189056 | 11467297 |
| dihydroergotamine | 5.46 | −0.725 | −0.209 | −0.231 | 0.841 | 0.611 | −1.645 | 0.412 | 0.132 | 0.881 | 3189057 | 11467298 |
| oleandomycin | 5.82 | 0.233 | 0.659 | −0.409 | −0.099 | −0.639 | −0.018 | 0.882 | 0.744 | 0.940 | 3189058 | 11467300 |
| midecamycin | 4.92 | 0.343 | 0.896 | −0.097 | −0.840 | −0.304 | 0.023 | 0.150 | −0.103 | 0.585 | 3189059 | 11467301 |
| paclitaxel | 4.68 | −0.352 | −1.276 | −2.576 | 0.116 | −2.010 | −2.115 | 0.949 | 0.708 | 1.198 | 3189060 | 11467303 |
| ivermectin | 4.58 | 1.161 | −0.341 | −0.291 | −0.399 | 1.009 | −0.498 | 0.172 | 0.099 | 0.242 | 3189061 | 11467304 |
| gentamicin sulfate | 2.88 | 0.282 | −0.078 | 1.215 | −0.159 | −0.043 | −0.598 | −0.261 | −0.551 | 0.334 | 3189062 | 11467308 |
| aztreonam | 9.18 | −0.470 | 1.025 | −1.241 | −0.942 | 1.032 | 0.273 | −0.408 | −0.571 | −0.033 | 3189063 | 11467333 |
| metaraminol | 12.6 | −0.545 | 0.255 | −1.192 | −0.885 | 1.545 | −0.275 | 0.171 | 0.082 | 0.274 | 3189174 | 11467345 |
| kawain | 17.38 | −0.826 | 0.038 | −1.416 | −1.000 | 0.871 | −0.681 | −0.157 | −0.280 | 0.089 | 3189175 | 11467355 |
| antimycin A | 7.3 | −0.827 | −2.124 | −1.958 | 1.345 | −0.522 | −0.969 | −1.672 | −1.695 | −1.341 | 3189176 | 11467370 |
| metampicillin | 11.06 | −0.887 | −0.115 | −1.317 | −0.251 | 0.596 | −1.458 | 0.191 | 0.044 | 0.400 | 3189177 | 11467383 |
| ethisterone | 12.8 | 0.267 | 0.413 | −0.499 | −0.358 | 0.302 | 0.300 | −0.791 | −0.715 | −0.796 | 3189178 | 11467409 |
| dimenhydrinate | 8.52 | −0.635 | 0.833 | −0.855 | −1.013 | 1.119 | 0.608 | 0.278 | 0.457 | −0.146 | 3189179 | 11467413 |
| prednisolone | 11.1 | −1.328 | −0.308 | −1.828 | −0.554 | 0.245 | −0.416 | −0.025 | −0.049 | 0.023 | 3189180 | 11467422 |
| enalapril | 10.62 | −0.217 | −0.181 | −1.135 | −0.279 | 0.063 | 0.141 | 0.135 | 0.147 | 0.086 | 3189181 | 11467462 |
| streptomycin | 6.88 | −0.709 | 1.103 | −0.569 | 0.421 | −0.156 | 1.292 | −0.195 | −0.020 | −0.503 | 3189183 | 11467469 |
| zidovudine | 14.92 | −0.277 | 2.049 | −1.301 | −0.832 | 0.693 | 0.611 | 0.221 | 0.245 | 0.133 | 3189185 | 11467481 |
| N6-methyladenosine | 14.22 | 0.521 | 0.971 | −0.288 | −0.239 | 0.965 | 0.596 | 0.803 | 0.957 | 0.337 | 3189186 | 11467486 |
| thioguanosine | 13.36 | −0.142 | 0.430 | −1.708 | −0.267 | 0.834 | 0.005 | 0.780 | 1.042 | 0.105 | 3189187 | 11467495 |
| amoxicillin | 10.94 | −1.094 | 0.476 | −0.685 | −0.693 | 1.552 | 0.139 | 0.314 | 0.298 | 0.294 | 3189189 | 11467505 |
| bambuterol | 10.88 | 0.478 | −0.493 | −0.552 | −0.875 | 0.748 | 0.143 | 0.453 | 0.345 | 0.599 | 3189191 | 11467509 |
| brinzolamide | 10.42 | −0.711 | 0.392 | −1.598 | −0.796 | 1.002 | −0.139 | 0.014 | 0.070 | −0.109 | 3189192 | 11467513 |
| methylergometrine | 11.78 | −1.118 | −0.424 | −1.251 | −0.948 | 1.095 | 0.182 | −0.599 | −0.558 | −0.565 | 3189193 | 11467522 |
| etoposide | 6.8 | −0.733 | −0.245 | −1.910 | −0.448 | −0.516 | −0.260 | 0.735 | 0.469 | 1.126 | 3189304 | 11467544 |
| oxantel | 6.62 | −0.307 | −0.303 | 0.069 | −1.789 | 2.405 | 0.178 | 0.807 | 0.605 | 1.067 | 3189305 | 11467546 |
| hesperidin | 6.56 | −0.109 | 0.592 | 0.329 | −0.156 | 0.911 | 0.353 | 0.459 | 0.197 | 0.892 | 3189306 | 11467548 |
| pepstatin A | 5.84 | 0.272 | 0.859 | −0.763 | −0.700 | 0.397 | 1.091 | −0.305 | −0.067 | −0.728 | 3189307 | 11467553 |
| androsterone | 13.78 | 0.293 | 0.555 | −0.542 | −0.544 | −0.164 | 0.841 | −1.093 | −0.771 | −1.544 | 3189308 | 11467559 |
| bacampicillin | 8.6 | 0.192 | 0.158 | −0.881 | −0.977 | 0.213 | 0.445 | 0.160 | 0.264 | −0.076 | 3189309 | 11467564 |
| calciferol | 10.08 | −0.074 | −0.555 | 0.282 | 0.381 | 0.970 | −1.054 | 0.001 | 0.279 | −0.562 | 3189310 | 11467568 |
| 7-aminocephalosporanic acid | 14.7 | −0.180 | 0.799 | −1.256 | −0.191 | 0.654 | 0.670 | −0.100 | 0.110 | −0.520 | 3189311 | 11467572 |
| cholecalciferol | 10.4 | −0.573 | −0.364 | −0.562 | −0.198 | 0.659 | −0.194 | 0.024 | 0.157 | −0.255 | 3189312 | 11467577 |
| cyanocobalamin | 2.94 | −1.085 | 0.632 | −1.478 | −0.846 | 0.328 | 0.329 | −1.209 | −0.985 | −1.421 | 3189313 | 11467581 |
| digitoxigenin | 10.68 | −0.888 | −0.339 | −1.229 | −0.512 | 0.438 | −0.073 | 0.114 | 0.247 | −0.179 | 3189314 | 11467584 |
| digoxin | 5.12 | −1.317 | 0.912 | −1.162 | −0.870 | 0.627 | 0.169 | 0.511 | 0.657 | 0.121 | 3189315 | 11467585 |
| gabazine | 13.92 | −0.773 | 0.684 | −1.804 | −0.345 | 0.313 | −0.094 | −0.494 | −0.441 | −0.502 | 3189316 | 11467591 |
| ginkgolide A | 9.8 | −0.812 | −0.907 | −1.701 | −0.670 | 0.029 | −0.161 | −0.704 | −0.408 | −1.173 | 3189317 | 11467592 |
| lactobionic acid | 11.16 | −0.062 | −0.424 | −1.181 | −0.327 | 0.417 | 0.269 | −0.083 | 0.142 | −0.540 | 3189433 | 11467600 |
| cefixime | 8.82 | 0.849 | 0.599 | −0.047 | −0.353 | 0.348 | 0.331 | −0.428 | −0.422 | −0.365 | 3189434 | 11467610 |
| N-acetylmuramic acid | 13.64 | 0.270 | −0.262 | −1.311 | −0.425 | −0.130 | −0.122 | −0.073 | 0.112 | −0.435 | 3189435 | 11467612 |
| cefotetan | 6.94 | 0.369 | −0.725 | −0.324 | −0.081 | −0.647 | 0.186 | 0.496 | 0.489 | 0.408 | 3189436 | 11467621 |
| puromycin | 8.48 | −5.640 | −8.529 | −6.198 | −3.529 | −3.921 | −5.832 | −1.590 | −2.800 | 1.240 | 3189437 | 11467628 |
| 6-azathymine | 31.48 | 1.057 | 2.625 | −0.772 | −0.350 | −0.053 | 0.583 | −0.464 | −0.176 | −0.964 | 3189438 | 11467631 |
| colistin | 3.46 | 0.940 | 1.795 | −1.283 | −0.746 | 0.501 | 0.409 | −0.341 | −0.016 | −0.939 | 3189439 | 11467634 |
| ceftazidime | 7.3 | 0.992 | 0.957 | −0.654 | −0.673 | −0.209 | 0.740 | −0.379 | −0.056 | −0.965 | 3189440 | 11467637 |
| terconazole | 7.52 | 0.751 | 0.127 | −3.209 | −0.196 | 1.691 | 0.552 | −1.032 | −0.870 | −1.158 | 3189441 | 11467643 |
| etifenin | 12.4 | 0.228 | 1.009 | −1.084 | −0.738 | 0.798 | 0.092 | −0.666 | −0.698 | −0.477 | 3189442 | 11467652 |
| nystatine | 4.32 | −0.430 | 0.370 | −1.147 | −0.660 | 0.765 | 0.061 | −0.436 | −0.272 | −0.683 | 3189443 | 11467665 |
| thiostrepton | 2.4 | −0.979 | −0.925 | −2.771 | −0.160 | −0.494 | −1.134 | 0.357 | −0.162 | 1.338 | 3189444 | 11467670 |
| rifampicin | 4.86 | −0.591 | −0.681 | −1.866 | −0.918 | −0.224 | 0.319 | −0.270 | −0.071 | −0.620 | 3189445 | 11467673 |
| thiocolchicoside | 7.1 | −0.498 | −0.617 | −0.850 | −0.168 | 1.037 | 0.108 | −0.170 | −0.099 | −0.282 | 3189446 | 11467687 |
| dirithromycin | 4.8 | −1.308 | −0.295 | −0.838 | −0.880 | 0.463 | 0.356 | −0.324 | −0.298 | −0.311 | 3189565 | 11467705 |
| tubocurarine | 6.56 | −1.217 | 2.248 | 0.659 | 0.044 | 0.229 | 1.117 | −0.267 | −0.426 | 0.104 | 3189566 | 11467709 |
| aconitine | 6.2 | −0.184 | 1.375 | −0.538 | −0.393 | 0.623 | 1.609 | 0.389 | 0.414 | 0.249 | 3189567 | 11467715 |
| emetine | 8.32 | −4.480 | 0.048 | −3.433 | 1.294 | −2.661 | −1.644 | −0.023 | 2.313 | −4.790 | 3189569 | 11467718 |
| tomatidine | 9.62 | −0.422 | 0.996 | −0.985 | −0.311 | 0.586 | 1.135 | −0.158 | 0.010 | −0.443 | 3189571 | 11467721 |
| (+)-chelidonine | 11.32 | −1.005 | −0.045 | −1.988 | −1.123 | −1.486 | −1.144 | 0.941 | 0.901 | 0.836 | 3189572 | 11467735 |
| tetrahydroalstonine | 11.34 | 0.055 | −0.199 | −1.505 | −1.309 | 1.058 | 1.439 | −0.219 | −0.294 | −0.016 | 3189573 | 11467741 |
| cinchonidine | 13.58 | −1.586 | −0.657 | −1.384 | −1.067 | 0.411 | −0.164 | −0.385 | −0.183 | −0.717 | 3189574 | 11467754 |
| canavanine | 22.7 | −0.510 | −0.289 | −1.333 | −0.640 | 0.992 | −0.321 | −0.178 | −0.262 | 0.032 | 3189575 | 11467757 |
| demecarium | 7.18 | 0.880 | −0.356 | −1.431 | −0.664 | 0.088 | −0.211 | −0.552 | −0.515 | −0.517 | 3189576 | 11467764 |
| cytisine | 21.02 | −0.534 | −0.789 | −0.118 | −0.704 | 1.404 | 0.095 | 0.025 | 0.181 | −0.295 | 3189578 | 11467772 |
| racecadotril | 10.38 | −0.468 | −0.182 | −1.550 | −0.382 | 0.705 | −0.249 | −0.249 | −0.229 | −0.235 | 3189579 | 11467774 |
| salsolinol | 22.32 | −1.127 | 0.469 | −1.113 | −0.160 | −0.248 | −0.994 | −0.284 | −0.197 | −0.397 | 3189580 | 11467776 |
| dimethisoquin | 14.68 | 0.300 | −0.047 | −0.838 | −0.222 | 1.731 | −1.006 | 0.302 | 0.140 | 0.575 | 3189581 | 11467778 |
| hydroquinine | 12.26 | 0.231 | 0.044 | −1.378 | −0.193 | 1.619 | 0.004 | 0.151 | −0.044 | 0.529 | 3189582 | 11467783 |
| avocatin A | 20 | 0.133 | 0.371 | −0.249 | −0.057 | 0.404 | 0.849 | −1.145 | −1.066 | −1.140 | 3198309 | 11487988 |
| (−)-duartin | 20 | 1.175 | 0.719 | −0.571 | 0.392 | 0.900 | −0.472 | −0.048 | −0.331 | 0.466 | 3198310 | 11488036 |
| fumarprotocetraric acid | 20 | −0.955 | 1.131 | −0.538 | −0.590 | 0.476 | 0.505 | −0.527 | −0.553 | −0.327 | 3198311 | 11488203 |
| canrenone | 20 | 0.025 | 0.952 | −0.644 | −0.307 | −0.063 | 0.816 | −0.663 | −0.842 | −0.123 | 3198312 | 11488300 |
| madecassic acid | 20 | 0.982 | 0.701 | −0.798 | 0.374 | −0.132 | 0.241 | 0.505 | 0.493 | 0.468 | 3198313 | 11488304 |
| telithromycin | 20 | 0.318 | −0.039 | −1.062 | −0.146 | 0.922 | −0.023 | −0.489 | −0.624 | −0.079 | 3198314 | 11488325 |
| deracoxib | 20 | 1.524 | 0.320 | −0.588 | −0.313 | 0.081 | 0.350 | 0.250 | 0.179 | 0.386 | 3198315 | 11488337 |
| troxerutin | 20 | −0.194 | −0.263 | −1.235 | −0.072 | 0.153 | −0.673 | −1.220 | −1.161 | −1.059 | 3198316 | 11488345 |
| trandoapril | 20 | 0.694 | 0.603 | −0.684 | −0.210 | 0.572 | 0.687 | −1.375 | −1.466 | −0.884 | 3198317 | 11488397 |
| zearalenone | 20 | 0.576 | 0.541 | −0.900 | −0.194 | 2.122 | −0.185 | −0.979 | −0.810 | −1.159 | 3198318 | 11488475 |
| securinine | 20 | −1.075 | 0.292 | −0.269 | 0.949 | −1.553 | −0.481 | 0.346 | 0.370 | 0.201 | 3198319 | 11488485 |
| oxiconazole | 20 | −3.228 | −3.585 | −1.535 | 1.299 | −1.503 | −3.509 | −1.477 | −1.697 | −0.747 | 3198320 | 11488514 |
| elaidylphosphocholine | 20 | −0.314 | −0.183 | −0.962 | −0.415 | 0.720 | −0.840 | −0.464 | −0.352 | −0.618 | 3198321 | 11488524 |
| cobalamine | 20 | −0.821 | −0.685 | −1.369 | 0.097 | 2.944 | −0.617 | −0.402 | −0.373 | −0.393 | 3198322 | 11488530 |
| derrustone | 20 | −0.982 | −0.355 | −1.694 | −0.126 | 0.664 | 0.470 | −0.764 | −0.685 | −0.797 | 3198323 | 11488579 |
| rutoside | 20 | −0.964 | 0.370 | −1.155 | −1.012 | 0.172 | −0.012 | −0.338 | −0.575 | 0.188 | 3198324 | 11488595 |
| 5,4′-dimethoxy-7-hydroxyflavone | 20 | −0.171 | −0.900 | −0.913 | −0.043 | 1.747 | 0.173 | 0.083 | −0.073 | 0.369 | 3198325 | 11488611 |
| endecaphyllin X | 20 | 0.158 | 0.525 | −1.007 | 0.142 | −0.292 | −0.219 | −0.660 | −0.566 | −0.741 | 3198326 | 11488622 |
| palmatine | 20 | −1.028 | 0.565 | −1.222 | −0.823 | 0.511 | −0.084 | 1.822 | 1.636 | 1.833 | 3198419 | 11488652 |
| vinblastine | 20 | 1.466 | −0.803 | −2.171 | 0.665 | −1.327 | −1.078 | 2.083 | 2.088 | 1.636 | 3198420 | 11488706 |
| pravastatin | 20 | −0.787 | 0.476 | −1.703 | −0.625 | 1.093 | 0.972 | −0.671 | −0.508 | −0.890 | 3198421 | 11488729 |
| hygromycin B | 20 | −1.526 | 0.370 | −2.041 | −0.752 | 0.707 | −0.478 | 0.558 | 0.800 | −0.054 | 3198422 | 11488734 |
| gemifloxacin | 20 | −0.578 | 0.647 | −1.445 | −1.057 | 0.992 | 0.009 | 0.256 | 0.287 | 0.203 | 3198423 | 11488908 |
| triflupromazine | 20 | −0.120 | −0.080 | −0.255 | −0.897 | 1.928 | 0.141 | 0.433 | 0.224 | 0.845 | 3198424 | 11488935 |
| topiramate | 20 | 0.452 | 1.483 | −0.732 | −0.212 | −0.360 | −0.093 | 0.255 | 0.302 | 0.178 | 3198425 | 11488944 |
| 4-amino-3-(5-chlorothien-2-yl)butanoic acid | 20 | 0.201 | −0.117 | −1.056 | −0.276 | 0.170 | 0.593 | 0.343 | 0.489 | 0.039 | 3198426 | 11488987 |
| nonoxynol-9 | 20 | −0.209 | −0.737 | −1.228 | −1.484 | 0.528 | 0.189 | −0.872 | −1.114 | −0.138 | 3198427 | 11489063 |
| trandolapril | 20 | −1.323 | 0.195 | −0.581 | −0.681 | 0.745 | 0.039 | −0.241 | −0.174 | −0.259 | 3198428 | 11489065 |
| megestrol acetate | 20 | 0.546 | 0.297 | 0.296 | −0.108 | 0.692 | 0.810 | 0.106 | 0.170 | 0.024 | 3198429 | 11489087 |
| TFA-Val-Tyr-Val-OH | 20 | −0.143 | 0.751 | −1.019 | −0.510 | 0.471 | −0.785 | 0.294 | 0.415 | −0.008 | 3198430 | 11489302 |
| valyltryptophan | 20 | −1.481 | −0.053 | −0.468 | −0.438 | 0.388 | −0.626 | −0.534 | −0.551 | −0.381 | 3198431 | 11489304 |
| S-methyl-L-thiocitrulline acetate | 20 | 0.613 | 1.060 | −0.262 | −0.542 | 0.052 | 0.533 | 0.291 | 0.325 | 0.173 | 3198432 | 11489307 |
| N-histidyl-2-aminonaphthalene | 20 | −0.137 | 0.371 | −0.147 | −0.618 | 0.353 | 0.214 | −0.595 | −0.469 | −0.739 | 3198433 | 11489308 |
| lysylphenylalanyltyrosine | 20 | −0.701 | 1.158 | −1.300 | −0.825 | 0.225 | 0.240 | −0.424 | −0.208 | −0.783 | 3198434 | 11489310 |
| phenylalanyltyrosine | 20 | −0.068 | 0.102 | −0.574 | −1.085 | 0.132 | −0.342 | −0.424 | −0.366 | −0.465 | 3198435 | 11489312 |
| selamectin | 20 | 0.300 | 0.856 | −0.348 | −0.467 | 0.641 | 0.343 | −0.112 | 0.126 | −0.565 | 3198436 | 11489399 |
| citicoline | 20 | −0.283 | 0.203 | −0.605 | −0.103 | 1.034 | 0.322 | −0.620 | −0.607 | −0.495 | 3198437 | 11489507 |
| icariin | 20 | −0.001 | 0.072 | −2.441 | −0.784 | 0.775 | 0.073 | 0.578 | 0.656 | 0.331 | 3198531 | 11489511 |
| oxfendazole | 20 | 0.107 | −0.524 | −2.568 | −0.446 | 0.692 | −0.224 | −0.801 | −0.626 | −0.969 | 3198532 | 11489520 |
| chlorophyllide | 20 | −0.178 | 0.439 | 0.307 | −0.783 | 1.042 | −1.157 | 0.282 | 0.303 | 0.224 | 3198533 | 11489524 |
| avocatin B | 20 | −0.869 | −0.292 | −1.119 | 0.024 | 1.229 | 0.710 | −0.935 | −0.863 | −0.852 | 3198534 | 11489534 |
| 1,3-dideacetyldeoxykhivorin | 20 | −0.409 | 0.176 | −0.507 | 0.283 | 1.279 | 0.007 | −0.370 | −0.392 | −0.207 | 3198535 | 11489535 |
| neopine | 20 | −1.240 | 0.172 | −1.068 | −0.640 | 0.784 | 0.421 | −0.701 | −0.866 | −0.183 | 3198536 | 11489561 |
| totarol-19-carboxylic acid | 20 | −0.506 | −1.295 | −1.701 | −0.008 | −0.209 | −1.116 | −0.399 | −0.494 | −0.073 | 3198537 | 11489564 |
| milldurone | 20 | −0.401 | 0.268 | −0.713 | −0.569 | 0.680 | 0.465 | 0.343 | 0.605 | −0.215 | 3198538 | 11489569 |
| gambogic acid | 20 | −5.389 | −8.341 | −6.656 | −4.102 | −4.200 | −5.596 | −3.830 | −4.010 | −2.680 | 3198539 | 11489625 |
| methyl gamboginate | 20 | −2.359 | 0.086 | −2.730 | −0.307 | 0.282 | −0.800 | −0.567 | −0.507 | −0.533 | 3198540 | 11489646 |
| lanosterol | 20 | 0.020 | 0.124 | −1.103 | −0.503 | 0.879 | 0.383 | −0.156 | −0.293 | 0.207 | 3198541 | 11489712 |
| dioonflavone | 20 | −0.003 | 0.062 | −0.744 | 0.208 | 0.650 | 0.605 | −0.471 | −0.204 | −0.969 | 3198542 | 11489742 |
| sodium salicylate | 20 | −0.836 | 0.123 | −0.890 | −0.996 | 0.435 | 0.607 | −0.164 | −0.086 | −0.341 | 3198543 | 11489761 |
| 3beta-hydroxy-23,24-bisnorchol-5-enic acid | 20 | −0.295 | 0.219 | −0.244 | −0.402 | 0.315 | −0.309 | 0.756 | 0.793 | 0.486 | 3198544 | 11489765 |
| p-hydroxycinnamaldehyde | 20 | −1.650 | 0.701 | −1.942 | −0.918 | 0.120 | −0.079 | −1.097 | −1.113 | −0.889 | 3198545 | 11489779 |
| geranyl cinnamate | 20 | −0.653 | 0.124 | −1.080 | −0.515 | 1.250 | −0.107 | −0.697 | −0.668 | −0.652 | 3198546 | 11489791 |
| telmisartan | 20 | 2.326 | −0.755 | −0.800 | −0.028 | 2.637 | −0.044 | −0.648 | −0.802 | −0.253 | 3198547 | 11489792 |
| 7-[2-trifluoromethyl-4-(2-hydroxyphenyl)-1,3- | 20 | −0.413 | 0.276 | −0.548 | −0.087 | 0.713 | −0.437 | 0.344 | 0.169 | 0.594 | 3198548 | 11489816 |
| dioxan-cis-5-yl]-hept-5z-enoic acid | | | | | | | | | | | | |
| diprotin A | 20 | −0.788 | −0.247 | −1.068 | 0.357 | 0.300 | −0.975 | 0.099 | −0.102 | 0.490 | 3198954 | 11489296 |
| bissalicyl | 20 | −0.317 | 1.023 | −1.349 | −0.161 | 0.356 | 0.551 | 0.047 | 0.014 | 0.046 | 3199904 | 11487821 |
| rifaximin | 20 | 1.755 | 0.212 | −0.813 | 0.910 | 1.073 | −0.471 | 0.411 | 0.282 | 0.541 | 3199905 | 11487836 |
| tylosin | 20 | −0.554 | 0.778 | −1.239 | −0.550 | 1.188 | −0.007 | −0.376 | −0.466 | −0.179 | 3199906 | 11487843 |
| sarafloxacin | 20 | −0.337 | −0.345 | −2.298 | −0.519 | 0.763 | 0.290 | 0.312 | 0.416 | −0.017 | 3199907 | 11487852 |
| atracurium | 4.3 | 0.005 | 1.412 | −0.223 | 0.509 | 0.755 | 1.176 | 0.530 | 0.626 | 0.182 | 3221455 | 11467153 |
| bacitracin | 2.82 | −0.938 | 0.616 | −0.310 | −0.214 | 0.105 | −0.806 | −0.592 | −0.736 | −0.189 | 3221506 | 11468067 |
| N-acetylaspartylglutamic acid | 20 | 0.010 | 0.503 | 0.280 | −0.393 | −0.197 | 0.480 | −0.126 | −0.008 | −0.342 | 3471022 | 11489297 |
| cephaloridine | 20 | −0.905 | 0.237 | −1.458 | −0.963 | 0.543 | −0.278 | −0.664 | −0.802 | −0.269 | 3471036 | 11488739 |
| cefsulodin | 20 | −0.348 | −0.481 | 0.132 | −0.784 | 1.553 | −1.078 | 0.455 | 0.516 | 0.204 | 3471107 | 11489766 |
| kanamycin A | 8.26 | −1.015 | −0.738 | −1.149 | −0.812 | 1.383 | −0.131 | −0.270 | −0.218 | −0.323 | 3471415 | 11467542 |
| ipratropium | 12.04 | −0.371 | 1.816 | −1.408 | −0.475 | 0.931 | 0.674 | 0.000 | 0.242 | −0.496 | 3471597 | 11467723 |
| isoxsuprine | 13.28 | −1.004 | −0.293 | −1.833 | −0.947 | 1.652 | −0.148 | 0.888 | 0.827 | 0.800 | 3471598 | 11467216 |
| metampicillin | 20 | 0.609 | 1.047 | −0.182 | −0.390 | −0.119 | 0.821 | −0.247 | −0.019 | −0.623 | 3471725 | 11488357 |
| bergenin | 12.18 | 0.555 | 0.898 | −1.415 | −0.637 | 0.880 | 0.831 | −0.379 | −0.421 | −0.232 | 3472295 | 11467959 |
| dehydroepiandrosterone | 20 | −0.999 | 0.584 | −0.731 | −0.137 | 0.596 | −0.634 | −0.139 | −0.151 | −0.033 | 3472730 | 11488175 |
| atovaquone | 10.9 | −0.567 | −1.627 | −2.368 | 0.488 | 0.773 | −0.124 | −0.505 | −0.281 | −0.870 | 3474094 | 11467682 |
| venlafaxine | 20 | 0.301 | 0.691 | −0.751 | −0.685 | 0.030 | 0.627 | −0.521 | −0.450 | −0.517 | 3474304 | 11488358 |
| gliclazide | 12.36 | −0.452 | −0.234 | −0.090 | −0.608 | 0.915 | 0.147 | −0.779 | −0.812 | −0.565 | 3474312 | 11467706 |
| mepivacaine | 20 | −0.497 | −0.150 | −0.686 | −0.945 | 0.411 | −0.084 | −0.780 | −0.625 | −0.907 | 3474314 | 11489472 |
| isradipine | 10.78 | −0.674 | −0.597 | −0.635 | 0.349 | 0.522 | 0.236 | 0.070 | −0.063 | 0.317 | 3480040 | 11468169 |
| ritodrine | 20 | −0.827 | 0.439 | −0.706 | −1.368 | 0.742 | −0.072 | −0.221 | −0.352 | 0.089 | 3480067 | 11489239 |
| pioglitazone | 20 | 0.749 | −1.224 | −0.352 | −0.421 | 1.512 | −0.742 | 0.579 | 0.500 | 0.657 | 3480074 | 11489495 |
| tolterodine | 20 | −0.490 | 0.228 | −0.729 | −1.032 | 0.472 | −0.406 | 0.049 | 0.113 | −0.050 | 3480078 | 11488342 |
| carvedilol | 20 | 0.453 | −4.716 | −5.765 | −3.375 | −2.321 | −4.745 | −2.460 | −2.817 | −1.246 | 3480079 | 11489489 |
| fexofenadine | 20 | −0.364 | 0.563 | −0.823 | −0.223 | 0.678 | −0.751 | 0.057 | 0.173 | −0.113 | 3480106 | 11488995 |
| sibutramine | 20 | −1.609 | −0.196 | −1.556 | 0.798 | 0.451 | 0.351 | 0.804 | 0.846 | 0.583 | 3480112 | 11489502 |
| procaterol | 20 | 0.002 | 0.084 | −1.049 | −0.363 | 0.795 | −0.168 | 0.094 | 0.298 | −0.336 | 3480197 | 11489366 |
| alfluzocin | 10.28 | 0.072 | 1.978 | −0.139 | −0.124 | −0.245 | −0.145 | −0.458 | −0.212 | −0.858 | 3480229 | 11467470 |
| alfluzocin | 20 | 0.315 | 1.565 | −1.047 | −0.209 | 0.139 | 0.781 | −0.237 | −0.339 | 0.063 | 3480229 | 11488321 |
| amlodipine | 20 | 0.030 | −0.359 | −1.287 | 0.003 | −0.179 | 0.902 | −1.328 | −1.512 | −0.655 | 3480246 | 11488302 |
| felodipine | 10.4 | 1.052 | 0.006 | −1.229 | 0.540 | −0.682 | −0.956 | 0.477 | 0.498 | 0.341 | 3480329 | 11467626 |
| rebamipide | 20 | −0.517 | 0.492 | −1.340 | −0.129 | 0.967 | −0.372 | −0.100 | −0.328 | 0.331 | 3480338 | 11487855 |
| bifonazole | 20 | −0.063 | 0.122 | −1.879 | −0.622 | 0.552 | 0.179 | −0.276 | −0.492 | 0.173 | 3480340 | 11487851 |
| azelastine | 20 | 0.001 | 1.381 | 0.174 | 1.404 | 0.268 | 0.054 | −0.315 | −0.467 | 0.015 | 3480341 | 11488465 |
| betaxolol | 13.02 | −0.889 | −0.479 | −0.648 | −0.507 | 0.759 | 0.064 | −0.317 | −0.309 | −0.273 | 3480396 | 11467530 |
| dobutamine | 13.28 | −0.245 | −0.138 | −1.063 | −1.373 | −0.132 | −0.289 | −0.285 | −0.059 | −0.683 | 3480458 | 11467500 |
| oxybutynin | 11.18 | −0.493 | 0.338 | −0.644 | −0.981 | 0.547 | −0.352 | 0.372 | 0.466 | 0.112 | 3480597 | 11467435 |
| naftopidil | 10.2 | 0.654 | −0.095 | −0.214 | 0.133 | 1.396 | 0.382 | 0.786 | 0.810 | 0.572 | 3480607 | 11468123 |
| sotalol | 14.68 | −0.754 | 0.583 | −0.390 | 0.105 | 0.305 | 0.501 | 0.744 | 0.885 | 0.299 | 3480627 | 11468114 |
| dipivefrin | 11.38 | 0.377 | 0.101 | −0.661 | −0.606 | 0.841 | −0.180 | 0.218 | 0.096 | 0.416 | 3487152 | 11467780 |
| nitrarine | 13.02 | −0.068 | −0.894 | −1.978 | −0.986 | 0.458 | 0.040 | −0.352 | −0.303 | −0.378 | 3487155 | 11467833 |
| proxyphylline | 16.78 | 0.659 | 2.332 | 0.395 | 0.821 | −0.186 | −0.574 | 0.111 | 0.174 | −0.056 | 3487233 | 11468038 |
| iodixanol | 2.58 | −0.580 | 0.017 | −2.238 | −0.651 | 1.280 | −0.092 | −0.422 | −0.534 | −0.114 | 3487254 | 11467996 |
| iopromide | 5.06 | −0.774 | 0.451 | −0.350 | 0.132 | 0.278 | 0.263 | 0.087 | 0.130 | −0.020 | 3487261 | 11468020 |
| ioversol | 4.96 | −0.567 | −1.318 | −0.842 | −0.693 | 0.319 | 0.129 | 0.110 | 0.000 | 0.310 | 3487262 | 11468026 |
| isoconazole | 9.62 | −0.442 | −0.920 | −2.216 | −0.823 | 1.342 | −0.572 | −1.056 | −1.233 | −0.525 | 3487389 | 11467275 |
| hydroxyzine | 10.66 | 0.793 | 1.036 | −1.043 | 0.143 | 0.020 | 0.258 | −1.077 | −0.878 | −1.323 | 3487390 | 11467281 |
| chlorphensin | 16.28 | −1.132 | −0.429 | −0.356 | −0.419 | 0.692 | −0.522 | −0.696 | −0.613 | −0.766 | 3487401 | 11467382 |
| bisoprolol | 12.3 | 0.314 | 1.423 | 0.031 | 0.986 | 0.513 | 0.874 | 0.633 | 0.607 | 0.564 | 3487402 | 11467478 |
| cisapride | 8.58 | 0.305 | 0.764 | 0.523 | 0.763 | 2.245 | −0.495 | −0.329 | −0.231 | −0.468 | 3487430 | 11467578 |
| tiaprofenic acid | 15.36 | −0.148 | 0.041 | −1.167 | −1.012 | 1.746 | 0.644 | 0.111 | 0.379 | −0.452 | 3487464 | 11467644 |
| cetirizine | 10.28 | 0.234 | 1.442 | −1.107 | −0.943 | 0.568 | 0.507 | −0.690 | −0.499 | −0.955 | 3487465 | 11467651 |
| syrosingopine | 6 | −0.660 | 2.649 | −0.077 | 0.479 | 0.586 | 1.051 | −0.123 | −0.010 | −0.336 | 3487467 | 11467712 |
| penbutolol | 13.72 | 0.287 | −0.825 | −1.020 | 0.436 | −0.960 | 0.099 | −1.251 | −1.184 | −1.143 | 3487585 | 11468191 |
| netilmicin | 8.42 | 0.633 | −0.654 | 0.883 | −0.582 | −0.762 | 0.156 | 1.032 | 0.861 | 1.167 | 3487604 | 11468249 |

TABLE 3
Top Compounds identified in screen

| | | | | | | ROS |
|---|---|---|---|---|---|---|
| Plate | Well | Compound Name | ChemBankID | PubChem_CID | GEHTS_Zscore | Zscore |
| 2163 | A04 | podophyllotoxin | 3177591 | 164791 | 3.506 | −1.656 |
| | | acetate | | | | |
| 2160 | P17 | podofilox | 1001531 | 10607 | 3.408 | −1.436 |
| 2160 | B22 | vinblastine sulfate | 3198420 | 6710780 | 3.171 | −1.327 |
| 2162 | I04 | mebendazole | 1350 | 4030 | 2.846 | −1.003 |
| 2161 | H22 | cytochalasin e | 3063989 | 6711190 | 2.827 | −2.109 |
| 2164 | D21 | Nocodazole | 440 | 4122 | 2.675 | −1.099 |
| 2158 | H15 | deoxysappanone b | 2060294 | 4026888 | 2.577 | −1.712 |
| | | 7,3′-dimethyl ether | | | | |
| 2160 | P05 | paclitaxel | 1000045 | 441276 | 2.451 | −1.967 |

TABLE 4
Compounds Clustered According to Structural Similarity

| | | | | | | | GE- | | | | |
|---|---|---|---|---|---|---|---|---|---|---|---|
| Cluster ID | Compound Name | Viability | ATP | MTT | ΔΨₘ | ROS | HTS | nucOX | mitoOX | CpdAnno | AssayAnno |
| A | nigericin | −1.744 | −3.165 | −3.329 | 1.324 | −3.323 | 15.99 | 8.185 | 3.884 | ionophore | low ROS |
| A | salinomycin | −0.247 | −3.542 | −2.918 | 2.976 | −3.111 | 16.48 | 8.804 | 3.303 | antibiotics | low mitoOX |
| A | lasalocid | −1.090 | −0.906 | −1.771 | −1.178 | −2.993 | 16.84 | 9.753 | 2.812 | | |
| A | monensin | −0.176 | −3.394 | −2.104 | 3.603 | −2.989 | 19.54 | 10.821 | 3.782 | | |
| A | lasalocid | −1.076 | −2.539 | −3.481 | −1.518 | −2.547 | 18.63 | 10.778 | 3.022 | | |
| A | monensin | −0.307 | −3.555 | −3.197 | 1.556 | −1.933 | 19.79 | 11.026 | 3.346 | | |
| A | heudelottin C | −0.829 | 0.840 | −0.393 | 0.373 | 0.576 | 18.96 | 9.187 | 5.034 | | |
| B | dihydrorobinetin | −1.070 | 0.456 | −1.259 | −3.396 | −0.393 | 18.80 | 9.311 | 5.214 | flavonoid | low ΔΨm |
| B | epiafzelechin(2r,3r)(−) | −0.719 | 0.986 | −0.486 | −3.118 | 1.019 | 20.49 | 9.928 | 5.722 | antioxidants | |
| B | baicalein | −0.456 | 1.556 | −1.632 | −2.552 | 0.851 | 21.13 | 10.195 | 6.020 | | |
| B | morin | −0.962 | 1.309 | −0.293 | −2.534 | 0.880 | 18.32 | 8.923 | 5.186 | | |
| B | quinalizarin | 0.583 | 1.276 | −0.668 | −2.511 | 0.674 | 20.11 | 9.896 | 5.429 | | |
| B | epigallocatechin | −1.489 | 0.559 | −1.197 | −2.502 | −0.531 | 19.18 | 9.279 | 5.345 | | |
| B | kaempferol | −0.013 | 0.060 | −0.352 | −2.485 | −1.322 | 18.55 | 8.957 | 5.283 | | |
| B | purpurin | −1.205 | −0.402 | −2.248 | −2.389 | 1.584 | 19.79 | 9.618 | 5.366 | | |
| B | epicatechin | −1.657 | 0.915 | −1.727 | −2.362 | 0.513 | 18.24 | 8.400 | 5.134 | | |
| B | epicatechin | 0.077 | 2.069 | −1.037 | −1.966 | −0.231 | 19.50 | 9.695 | 5.538 | | |
| B | catechin hydrate | 0.165 | 1.848 | −1.301 | −1.926 | 0.880 | 19.43 | 9.516 | 5.237 | | |
| B | hieracin | 1.425 | 0.806 | −0.557 | −1.902 | −0.329 | 18.19 | 8.966 | 5.035 | | |
| B | cianidanol | 0.630 | 1.283 | −1.471 | −1.746 | −0.371 | 18.69 | 8.815 | 5.387 | | |
| B | quercetin | 0.816 | 0.716 | −1.595 | −1.691 | 0.374 | 19.48 | 9.572 | 5.157 | | |
| B | fisetinidol | −1.412 | 0.542 | −1.804 | −1.528 | −0.215 | 17.44 | 8.389 | 5.079 | | |
| B | luteolin | −0.627 | −0.637 | −0.209 | −1.473 | 0.247 | 20.55 | 9.757 | 5.734 | | |
| B | quercetin | 0.686 | 0.361 | −0.928 | −1.240 | 0.615 | 19.22 | 9.135 | 5.517 | | |
| B | hematein | −0.357 | −0.036 | −1.726 | −1.215 | 0.835 | 19.45 | 9.571 | 5.445 | | |
| B | haematoxylin | −1.044 | 0.382 | −1.335 | −1.035 | 0.225 | 19.97 | 10.054 | 5.184 | | |
| B | fisetin | −0.764 | 0.456 | −0.689 | −0.893 | 0.442 | 20.40 | 9.947 | 5.701 | | |
| B | 1,4,5,8-tetrahydroxy-2,6- | −0.853 | −0.346 | −0.344 | −0.662 | 0.772 | 19.02 | 9.187 | 5.408 | | |
| | dimethylanthroquinone | | | | | | | | | | |
| B | hesperetin | −0.764 | 0.123 | −1.157 | −0.650 | 0.147 | 20.24 | 10.051 | 5.339 | | |
| B | hesperetin | −0.860 | −0.368 | −1.684 | −0.633 | 0.674 | 18.29 | 9.086 | 5.049 | | |
| B | naringenin | 0.350 | 0.892 | −0.475 | −0.620 | 0.735 | 19.92 | 9.713 | 5.678 | | |
| B | brazilin | −1.426 | 1.655 | −1.218 | −0.602 | −1.481 | 18.48 | 9.653 | 4.329 | | |
| B | brazilein | −1.633 | 0.713 | −0.725 | −0.110 | 1.078 | 21.92 | 10.798 | 5.914 | | |
| B | naringenin | −1.071 | 1.514 | −0.626 | 0.510 | 0.248 | 18.54 | 9.387 | 4.838 | | |
| B | rhamnetin | −1.211 | 1.672 | −0.975 | 0.717 | 0.228 | 18.77 | 9.107 | 4.892 | | |
| C | methiazole | −0.683 | −0.555 | −0.654 | 0.040 | −2.515 | 21.38 | 10.254 | 6.329 | azole | low ROS |
| C | parbendazole | 0.211 | −1.119 | −1.477 | 0.418 | −1.879 | 22.47 | 10.896 | 6.039 | antifungals | high GE- |
| | | | | | | | | | | | HTS |
| C | albendazole | −0.402 | 0.854 | −1.894 | −0.296 | −1.434 | 22.89 | 11.463 | 6.157 | | |
| C | oxibendazole | −1.899 | −0.104 | −3.046 | −0.975 | −1.178 | 22.00 | 10.650 | 6.001 | | |
| C | nocodazole | −0.069 | −0.969 | −0.751 | 0.358 | −1.099 | 24.00 | 11.901 | 6.018 | | |
| C | mebendazole | 0.512 | −0.483 | −2.060 | −0.434 | −1.003 | 23.99 | 11.892 | 6.199 | | |
| C | fenbendazole | −1.112 | −0.672 | −3.590 | −0.395 | −0.919 | 19.84 | 9.743 | 5.662 | | |
| C | fenbendazole | −0.398 | −1.895 | −3.769 | −0.360 | −0.797 | 20.00 | 9.639 | 5.307 | | |
| C | mebendazole | 0.064 | 0.344 | −2.797 | −0.054 | −0.791 | 20.64 | 10.038 | 5.807 | | |
| C | oxfendazole | 0.107 | −0.524 | −2.568 | −0.446 | 0.692 | 19.75 | 9.847 | 5.320 | | |
| D | tetracycline | −0.461 | 0.284 | 0.314 | −1.031 | 0.987 | 21.80 | 10.191 | 6.473 | tetracycline | high mitoOX |
| D | tetracycline | −0.405 | −0.099 | 0.144 | −0.854 | 0.275 | 20.20 | 9.449 | 6.270 | antibiotics | |
| D | oxytetracycline | 0.029 | −0.781 | −1.129 | −1.245 | 0.484 | 20.78 | 9.797 | 6.201 | | |
| D | minocycline | −0.640 | 1.387 | −1.103 | 0.117 | 1.514 | 20.98 | 10.284 | 5.684 | | |
| D | demeclocycline | 0.335 | −0.475 | −0.417 | −0.818 | 0.335 | 21.29 | 10.237 | 6.045 | | |
| D | methacycline | −1.645 | −0.365 | −1.744 | −1.616 | 1.083 | 20.33 | 9.809 | 5.879 | | |
| D | doxycycline | −0.322 | −0.568 | −1.407 | −1.355 | 0.943 | 18.86 | 8.814 | 5.904 | | |
| D | methacycline | 0.323 | 2.538 | −0.031 | −0.517 | 0.261 | 20.94 | 10.237 | 5.945 | | |
| D | oxytetracycline | 0.398 | 1.384 | −1.134 | 0.039 | 0.702 | 20.51 | 9.899 | 5.871 | | |
| D | doxycycline | −0.338 | −0.232 | −1.655 | −1.288 | 1.003 | 20.22 | 9.380 | 5.847 | | |
| D | chlortetracycline | −0.009 | −0.150 | −0.740 | −0.228 | 0.234 | 21.40 | 10.451 | 5.743 | | |
| D | demeclocycline | −0.733 | −0.208 | −2.363 | −1.613 | 0.110 | 19.66 | 9.508 | 5.697 | | |
| D | minocycline | −0.150 | 1.286 | −0.919 | −0.905 | 1.108 | 20.42 | 9.867 | 6.073 | | |
| D | chlortetracycline | 0.391 | 0.875 | 0.026 | 0.339 | −0.390 | 19.78 | 9.695 | 5.244 | | |
| E | dihydrorotenone | 0.390 | −5.350 | −4.020 | −0.314 | −0.682 | 14.76 | 6.664 | 4.180 | rotenone | low ATP |
| E | isorotenone | −1.954 | −5.273 | −4.373 | −1.329 | −0.782 | 12.12 | 5.742 | 2.595 | derivatives | |
| E | deguelin(−) | −0.940 | −5.123 | −4.052 | 1.451 | 0.426 | 16.15 | 7.372 | 4.719 | | |
| E | rotenone | −2.235 | −4.842 | −4.233 | −2.300 | −0.346 | 16.19 | 8.115 | 4.027 | | |
| E | mundulone | −1.113 | −4.544 | −2.487 | 3.037 | −0.141 | 15.32 | 7.065 | 4.571 | | |
| E | alpha-toxicarol | 0.304 | −2.946 | −1.982 | 0.820 | 0.101 | 21.85 | 10.719 | 5.672 | | |
| E | beta-dihydrorotenone | −0.184 | −2.497 | −2.771 | 1.079 | 0.122 | 17.81 | 8.284 | 4.802 | | |
| E | griseofulvin | 0.008 | −2.024 | −1.919 | 0.065 | −1.037 | 19.49 | 9.294 | 5.612 | | |
| E | beta-toxicarol | 0.672 | −2.000 | −1.498 | 0.899 | −0.188 | 16.36 | 7.629 | 5.026 | | |
| E | 2-ethoxycarbonyl-2- | 0.511 | −1.500 | −0.260 | 0.215 | 0.774 | 19.83 | 9.493 | 5.331 | | |
| | hydroxy-5,7- | | | | | | | | | | |
| | dimethoxyisoflavanone | | | | | | | | | | |
| E | griseofulvic acid | 0.176 | −1.279 | −0.469 | −0.666 | −0.323 | 18.50 | 8.707 | 5.064 | | |
| E | mundoserone | −0.425 | −1.275 | −1.681 | 0.837 | −0.608 | 17.96 | 8.461 | 4.891 | | |
| E | picropodophyllotoxin | −0.107 | −0.952 | −2.402 | −0.349 | −1.202 | 23.13 | 11.490 | 6.009 | | |
| E | dihydromundulone | 0.816 | −0.792 | 0.189 | −0.530 | 0.537 | 23.53 | 11.135 | 5.945 | | |
| E | podofilox | 0.789 | −0.716 | −1.507 | −0.212 | −1.436 | 24.99 | 12.347 | 6.311 | | |
| E | methyl robustone | 0.848 | −0.638 | −0.344 | 0.715 | −0.347 | 20.15 | 9.540 | 5.541 | | |
| E | robustic acid methyl ether | 0.497 | −0.589 | 0.474 | 0.692 | 1.431 | 21.93 | 11.080 | 5.749 | | |
| E | ichthynone | 0.234 | −0.541 | −0.681 | −0.325 | 1.842 | 19.22 | 9.346 | 5.345 | | |
| E | podophyllotoxin | −0.077 | −0.405 | −1.865 | −0.902 | −1.401 | 23.69 | 12.039 | 5.764 | | |
| E | 12a-hydroxy-5- | −0.940 | −0.089 | −1.976 | −0.179 | 0.823 | 22.35 | 10.784 | 5.999 | | |
| | deoxydehydromunduserone | | | | | | | | | | |
| E | isoduartin methyl ether | −0.603 | 0.032 | −2.055 | −0.777 | 0.599 | 20.56 | 10.192 | 5.414 | | |
| E | dehydrodihydrorotenone | −0.172 | 0.084 | −3.034 | 0.125 | 0.051 | 19.08 | 8.887 | 5.481 | | |
| E | dihydrosamidin | 0.476 | 0.085 | −0.826 | 0.199 | 4.794 | 23.98 | 11.603 | 6.477 | | |
| E | robustic acid | −1.213 | 0.257 | −1.831 | −1.086 | −0.119 | 18.87 | 9.064 | 5.314 | | |
| E | 8-iodocatechin tetramethyl | 0.377 | 0.343 | −1.359 | 0.146 | −0.448 | 17.34 | 8.295 | 4.791 | | |
| | ether | | | | | | | | | | |
| E | duartin(−) | 1.175 | 0.719 | −0.571 | 0.392 | 0.900 | 18.71 | 8.861 | 5.480 | | |
| E | rotenonic acid | 0.206 | 0.763 | −0.731 | 0.050 | 0.173 | 19.46 | 9.652 | 5.304 | | |
| E | dehydrorotenone | −0.381 | 0.836 | −1.046 | 0.884 | 0.804 | 22.23 | 10.898 | 5.629 | | |
| E | quassin | −0.688 | 0.866 | −0.400 | 0.033 | 0.925 | 20.00 | 9.699 | 5.513 | | |
| E | dihydrorobustic acid | −0.721 | 2.345 | −0.780 | 0.000 | −0.115 | 18.87 | 9.266 | 5.181 | | |

TABLE 5
Microtubule modulators in screened collection

| | | | ChemBank | | | ROS |
|---|---|---|---|---|---|---|
| Plate | Well | Compound Name | ID | PubChem_CID | GEHTS_Zscore | Zscore |
| 2160 | P17 | podofilox | 1001531 | 10607 | 3.408 | −1.436 |
| 2160 | B22 | vinblastine sulfate | 3198420 | 6710780 | 3.171 | −1.327 |
| 2162 | I04 | mebendazole | 1350 | 4030 | 2.846 | −1.003 |
| 2164 | D21 | Nocodazole | 440 | 4122 | 2.675 | −1.099 |
| 2166 | N05 | Podophyllotoxin | 1001531 | 10607 | 2.561 | −1.401 |
| 2160 | P05 | paclitaxel | 1000045 | 441276 | 2.451 | −1.967 |
| 2165 | A15 | Albendazole | 1185085 | 2082 | 2.379 | −1.434 |
| 2159 | H21 | Picropodophyllo- | 2069291 | 72435 | 2.259 | −1.202 |
| | | toxin | | | | |
| 2164 | N14 | Griseofulvin | 3069117 | 6713927 | 2.199 | 0.371 |
| 2164 | P11 | Paclitaxel; taxol | 3189060 | 6713921 | 2.118 | −2.010 |
| 2158 | O11 | Colchicine | 1352 | 6167 | 1.215 | −0.377 |
| 2165 | I08 | Colchicine | 1352 | 6167 | 0.901 | −0.345 |
| 2164 | L16 | Mebendazole | 1350 | 4030 | 0.623 | −0.791 |

TABLE 6
Sappanone derivatives in screened collection

| | | | ChemBank | | | ROS |
|---|---|---|---|---|---|---|
| Plate | Well | Compound Name | ID | PubChem_CID | GEHTS_Zscore | Zscore |
| 2158 | H15 | deoxysappanone | 2060294 | 4026888 | 2.577 | −1.712 |
| | | (B) 7,3′-dimethyl | | | | |
| | | ether | | | | |
| 2164 | K15 | sappanone (A) | 2060467 | 3288218 | 2.376 | 0.1195 |
| | | trimethyl ether | | | | |
| 2163 | E21 | 3- | 2060307 | 6708683 | 1.970 | 1.3852 |
| | | deshydroxysappanol | | | | |
| | | trimethyl ether | | | | |
| 2158 | L06 | sappanone (A) 7- | 3077175 | 6710767 | 1.410 | −1.493 |
| | | methyl ether | | | | |
| 2158 | F15 | deoxysappanone | 2060494 | 4643334 | 0.768 | 1.064 |
| | | (B) trimethyl ether | | | | |
| 2163 | P22 | tetrahydrosappanone | 2060448 | 6708784 | 0.583 | 0.274 |
| | | (A) trimethyl | | | | |
| | | ether | | | | |
| 2160 | D05 | sappanone (A) | 2060397 | 3884104 | 0.524 | −2.429 |
| | | dimethyl ether | | | | |
| 2158 | D19 | deoxysappanone | 3054809 | 6708755 | −0.723 | −1.894 |
| | | (B) 7,3′-dimethyl | | | | |
| | | ether acetate | | | | |

TABLE 7
Differential expression of nuclear and mtDNA OXPHOS genes

| | | | | mito- | |
|---|---|---|---|---|---|
| | | nucOXPHOS | | OXPHOS | |
| Rank | Compound Name | Z | Rank | Z | Rank |

High Nuclear/High Mitochondrial

| | | | | | |
|---|---|---|---|---|---|
| 1 | Prieurianin | 1.90 | 12 | 1.93 | 14 |
| 2 | Palmatine | 1.64 | 17 | 1.83 | 17 |
| 3 | vinblastine sulfate | 2.09 | 8 | 1.64 | 28 |
| 4 | anhydrobrazilic acid | 1.56 | 23 | 1.68 | 21 |
| 5 | Minoxidil | 2.38 | 2 | 1.37 | 44 |
| 6 | deoxysappanone b 7,3′-dimethyl | 2.25 | 6 | 1.38 | 42 |
| | ether | | | | |
| 7 | Dihydrosamidin | 1.84 | 14 | 1.48 | 37 |
| 8 | Indomethacin | 1.32 | 46 | 2.40 | 10 |
| 9 | Podofilox | 2.53 | 1 | 1.28 | 57 |
| 10 | Fluorouracil | 1.50 | 28 | 1.55 | 32 |

High Nuclear/Low Mitochondrial

| | | | | | |
|---|---|---|---|---|---|
| 1 | Emetine | 2.31 | 3 | −4.79 | 12 |
| 2 | Dihydrocelastrol | 2.18 | 7 | −5.22 | 9 |
| 3 | Emetine | 1.93 | 10 | −4.15 | 19 |
| 4 | Crinamine | 2.27 | 5 | −2.89 | 34 |
| 5 | Lycorine | 1.86 | 13 | −3.34 | 27 |
| 6 | Cycloheximide | 1.35 | 41 | −4.18 | 17 |
| 7 | Anisomycin | 1.26 | 52 | −3.55 | 23 |
| 8 | Lycorine | 1.10 | 74 | −5.39 | 5 |
| 9 | Cycloheximide | 0.69 | 238 | −5.64 | 3 |
| 10 | Monensin | 0.70 | 231 | −4.13 | 20 |

Low Nuclear/High Mitochondrial

| | | | | | |
|---|---|---|---|---|---|
| 1 | Perphenazine | −4.32 | 2 | 3.42 | 4 |
| 2 | Menadione | −3.40 | 17 | 2.13 | 12 |
| 3 | Chloranil | −2.45 | 37 | 2.50 | 8 |
| 4 | Triptonide | −2.18 | 44 | 5.02 | 2 |
| 5 | cetrimonium bromide | −2.70 | 29 | 1.66 | 26 |
| 6 | Doxorubicin | −1.85 | 57 | 2.57 | 7 |
| 7 | Puromycin | −2.80 | 26 | 1.24 | 67 |
| 8 | Daunorubicin | −2.09 | 49 | 1.37 | 45 |
| 9 | Disulfiram | −4.10 | 6 | 0.93 | 152 |
| 10 | Thimerosal | −1.20 | 178 | 10.41 | 1 |

Low Nuclear/Low Mitochondrial

| | | | | | |
|---|---|---|---|---|---|
| 1 | Isorotenone | −3.02 | 21 | −4.57 | 14 |
| 2 | Neostigmine | −3.54 | 15 | −3.45 | 24 |
| 3 | Pararosaniline | −2.25 | 40 | −5.43 | 4 |
| 4 | gambogic acid amide | −4.01 | 10 | −2.68 | 38 |
| 5 | 1- | −4.03 | 8 | −2.48 | 44 |
| | benzyloxycarbonylaminophenethyl | | | | |
| | chloromethyl ketone | | | | |
| 6 | 3,4-dimethoxydalbergione | −2.94 | 22 | −2.72 | 36 |
| 7 | Pyrromycin | −2.89 | 23 | −2.67 | 39 |
| 8 | Perphenazine | −3.10 | 19 | −2.46 | 45 |
| 9 | Quinacrine | −2.65 | 30 | −2.81 | 35 |
| 10 | pyrvinium pamoate | −2.08 | 50 | −4.40 | 15 |

TABLE 8
Summary of glucose uptake after paclitaxel treatment

| Treatment Duration | 1 nM paclitaxel | p-value | 1 μM paclitaxel | p-value |
|---|---|---|---|---|
| 30 minutes | 1.11 ± 0.03 | 0.009 | 1.19 ± 0.05 | 0.027 |
| 3 hours | 1.18 ± 0.05 | 0.035 | 1.23 ± 0.19 | 0.065 |

Fold change in basal glucose uptake rate compared to DMSO control.
Experiments performed in C2C12 myotubes.
P-values correspond to T-test (two-tailed).

TABLE 9
Tag Sequences and Universal Primers

| | | |
|---|---|---|
| Tag ID | Tag Sequence | SEQ ID |

| | | |
|---|---|---|
| 1 | CTTTAATCTCAATCAATACAAATC | SEQ ID NO: 71 |
| 2 | CTTTATCAATACATACTACAATCA | SEQ ID NO: 72 |
| 3 | TACACTTTATCAAATCTTACAATC | SEQ ID NO: 73 |
| 4 | TACATTACCAATAATCTTCAAATC | SEQ ID NO: 74 |
| 6 | TCAACAATCTTTTACAATCAAATC | SEQ ID NO: 75 |
| 7 | CAATTCATTTACCAATTTACCAAT | SEQ ID NO 76 |
| 8 | AATCCTTTTACATTCATTACTTAC | SEQ ID NO: 77 |
| 9 | TAATCTTCTATATCAACATCTTAC | SEQ ID NO 78 |
| 10 | ATCATACATACATACAAATCTACA | SEQ ID NO. 79 |
| 11 | TACAAATCATCAATCACTTTAATC | SEQ ID NO: 80 |
| 15 | ATACTTCATTCATTCATCAATTCA | SEQ ID NO: 81 |
| 18 | TCAAAATCTCAAATACTCAAATCA | SEQ ID NO: 82 |
| 19 | TCAATCAATTAC1TACTCAAATAC | SEQ ID NO: 83 |
| 31 | TTCACTTTTCAATCAACTTTAATC | SEQ ID NO: 84 |
| 32 | ATTATTCACTTCAAACTAATCTAC | SEQ ID NO: 85 |
| 33 | TCAATTACTTCACTTTAATCCTTT | SEQ ID NO: 86 |
| 34 | TCATTCATATACATACCAATTCAT | SEQ ID NO: 87 |
| 35 | CAATTTCATCATTCATTCATTTCA | SEQ ID NO: 88 |
| 36 | CAATTCATTTCATTCACAATCAAT | SEQ ID NO: 89 |
| 37 | CTTTTCATCTTTTCATCTTTCAAT | SEQ ID NO: 90 |
| 38 | TCAATCATTACACTTTTCAACAAT | SEQ ID NO: 91 |
| 40 | CTTTCTACATTATTCACAACATTA | SEQ ID NO: 92 |
| 42 | CTATCTTCATATTTCACTATAAAC | SEQ ID NO: 93 |
| 43 | CTTTCAATTACAATACTCATTACA | SEQ ID NO: 94 |
| 44 | TCATTTACCAATCTTTCTTTATAC | SEQ ID NO: 95 |
| 45 | TCATTTCACAATTCAATTACTCAA | SEQ ID NO: 96 |
| 46 | TACATCAACAATTCATTCAATACA | SEQ ID NO: 97 |
| 47 | CTTCTCATTAACTTACTTCATAAT | SEQ ID NO: 98 |
| 48 | AAACAAACTTCACATCTCAATAAT | SEQ ID NO: 99 |
| 49 | TCATCAATCTTTCAATTTACTTAC | SEQ ID NO: 100 |
| 50 | CAATATACCAATATCATCATTTAC | SEQ ID NO: 101 |
| 51 | TCATTTCAATCAATCATCAACAAT | SEQ ID NO: 102 |
| 52 | TCAATCATCTTTATACTTCACAAT | SEQ ID NO: 103 |
| 64 | CTACATATTCAAATTACTACTTAC | SEQ ID NO: 104 |
| 65 | CTTTTCATCAATAATCTTACCTTT | SEQ ID NO: 105 |
| ID | Universal Primers | |
| T7 | TAATACGACTCACTATAGGG | SEQ ID NO: 106 |
| T3 | TCCCTTTAGTGAGGGTTAAT | SEQ ID NO: 107 |

TABLE 10
Probes used in GE-HTS

| | |
|---|---|
| Gene Name | SEQ ID NO |

| | | |
|---|---|---|
| | Upstream primer full sequence (5′-3′) | |
| | [T7sequence-Tag-UpstreamTargetSequence] | |
| Mt-Atp6 | TAATACGACTCACTATAGGG-CAATTCATTTACCAATTTACCAAT-TTCAAGCCTACGTATTCACC | SEQ ID NO: 108 |
| Mt-Atp8 | TAATACGACTCACTATAGGG-TCAATCATTACACTTTTCAACAAT-TCACCAAAATCACTAACAAC | SEQ ID NO: 109 |
| Mt-Co1 | TAATACGACTCACTATAGGG-AATCCTTTTACATTCATTACTTAC-CACGACGCTACTCAGACTAC | SEQ ID NO: 110 |
| Mt-Co2 | TAATACGACTCACTATAGGG-CTTTCTACATTATTCACAACATTA-AACAAACGACCTAAAACCTG | SEQ ID NO: 111 |
| Mt-Co3 | TAATACGACTCACTATAGGG-CTATCTTCATATTTCACTATAAAC-TAGGACTTTACTTCACCATC | SEQ ID NO: 112 |
| Mt-Cytb | TAATACGACTCACTATAGGG-TAATCTTCTATATCAACATCTTAC-CTAATACCTTTCCTTCATAC | SEQ ID NO: 113 |
| Mt-Nd1 | TAATACGACTCACTATAGGG-ATCATACATACATACAAATCTACA-TACTACTATCATCAACATTC | SEQ ID NO: 114 |
| Mt-Nd2 | TAATACGACTCACTATAGGG-CTTTCAATTACAATACTCATTACA-TTCTTCCTTACAACCCATCC | SEQ ID NO: 115 |
| Mt-Nd3 | TAATACGACTCACTATAGGG-TCATTTACCAATCTTTGTTTATAC-TTACATTTCTATTATTTGAC | SEQ ID NO: 116 |
| Mt-Nd4 | TAATACGACTCACTATAGGG-TCATTTCACAATTCAATTACTCAA-ACTACGAACGGATCCACAGC | SEQ ID NO: 117 |
| Mt-Nd4I | TAATACGACTCACTATAGGG-TACATCAACAATTCATTCAATACA-ATTATAACTTCAGTAACTTC | SEQ ID NO: 118 |
| Mt-Nd5 | TAATACGACTCACTATAGGG-CTTCTCATTAACTTAGTTCATAAT-CCTACTAATTACACTAATCG | SEQ ID NO: 119 |
| Mt-Nd6 | TAATACGACTCACTATAGGG-TACAAATCATCAATCACTTTAATC-GAGATTGGTTGATGTATGAG | SEQ ID NO: 120 |
| Atp5a1 | TAATACGACTCACTATAGGG-CTTTAATCTCAATCAATACAAATC-AAAGGGTTACTCTTGTATTC | SEQ ID NO: 121 |
| Atp5c1 | TAATACGACTCACTATAGGG-CTTTATCAATACATACTACAATCA-CTTGACTTTCAACCGCACCC | SEQ ID NO: 122 |
| Atp5o | TAATACGACTCACTATAGGG-TTCACTTTTCAATCAACTTTAATC-GCTGAAGAGCTTCCTGAGTC | SEQ ID NO: 123 |
| Cox5b | TAATACGACTCACTATAGGG-TACACTTTATCAAATCTTACAATC-CCAAAGGCAGCTTCAGGCAC | SEQ ID NO: 124 |
| Cox7a2 | TAATACGACTCACTATAGGG-TCAATTACTTCAGTTTAATCCTTT-CCAATAAAGCAATCCTTAAC | SEQ ID NO: 125 |
| Cyc1 | TAATACGACTCAGTATAGGG-ATTATTCACTTCAAACTAATCTAC-TTTCCCGGCCAGGCCATTGG | SEQ ID NO: 126 |
| Hspc051 | TAATACGACTCACTATAGGG-TCATTCATATACATACCAATTCAT-TAAGGATGAGTTTCAAGTTG | SEQ ID NO: 127 |
| Ndufa5 | TAATACGAGTCACTATAGGG-TACATTACCAATAATCTTCAAATC-TGATATTCTGAAGCACTTTC | SEQ ID NO: 128 |
| Ndufb5 | TAATACGACTCACTATAGGG-CAATTTCATCATTCATTCATTTCA-CTGTGCAAGAACAGTGTGTC | SEQ ID NO: 129 |
| Sdhd | TAATACGACTCACTATAGGG-CAATTCATTTCATTCACAATCAAT-TTTAGACAAGTTCAATTTAG | SEQ ID NO: 130 |
| Uqcrb | TAATACGACTCACTATAGGG-CTTTTCATCTTTTCATGTTTCAAT-CTGGATGGTTTTCGAAAGTG | SEQ ID NO: 131 |
| Uqorc1 | TAATACGACTCACTATAGGG-TCAACAATCTTTTACAATCAAATC-TCCCAGACTACAACCGGATC | SEQ ID NO: 132 |
| Actb | TAATACGACTCACTATAGGG-AAACAAACTTCACATCTCAATAAT-TAAGTGGTTACAGGAAGTCC | SEQ ID NO: 133 |
| Aamp | TAATACGACTCACTATAGGG-CAATATACCAATATCATCATTTAC-GGGTGCGTCTTTGTATGTTG | SEQ ID NO: 134 |
| Cenpb | TAATACGACTCACTATAGGG-ATACTTCATTCATTCATCAATTCA-GTCCAGCCACCCAGGTGCTC | SEQ ID NO: 135 |
| Eef1a1 | TAATACGACTCACTATAGGG-TCAATCAATTACTTACTCAAATAG-ATAACAATGCATCGTAAAAC | SEQ ID NO: 136 |
| Jund | TAATACGACTCACTATAGGG-TCAATCATCTTTATACTTGACAAT-CCGCCTCTCTACCCGGAGTC | SEQ ID NO: 137 |
| Lsp1 | TAATACGACTCACTATAGGG-TCATTTCAATCAATCATCAACAAT-TGACCAACGCTCCAACTCTG | SEQ ID NO: 138 |
| Rps2 | TAATACGACTCACTATAGGG-TCAAAATCTCAAATACTCAAATCA-ACGGATCATCTTGTGAAAAC | SEQ ID NO: 139 |
| Rps27a | TAATACGACTCACTATAGGG-TCATCAATCTTTGAATTTACTTAC-TGGTAAGCAGCTGGAAGATG | SEQ ID NO: 140 |
| Cyb5r3 | TAATACGACTCACTATAGGG-CTTTTCATCAATAATGTTACCTTT-ACTCCATGCAGTCTTGAGTG | SEQ ID NO: 141 |
| EN1 | TAATACGACTCACTATAGGG-CTACATATTCAAATTACTACTTAC-TTCTCTGAAACGCAGGATTG | SEQ ID NO: 142 |
| | Downstream primer full sequence (5′-3′) | |
| | [DownstreamTargetSequence-T3-sequence] | |
| Mt-Atp6 | /5Phos/CTCCTAGTAAGCCTATATCT-TCCCTTTAGTGAGGGTTAAT | SEQ ID NO: 143 |
| Mt-Atp8 | /5Phos/CATAAAAGTAAAAACCCCTT-TCCCTTTAGTGAGGGTTAAT | SEQ ID NO: 144 |
| Mt-Co1 | /5Phos/CCAGATGCTTACACCACATG-TCCCTTTAGTGAGGGTTAAT | SEQ ID NO: 145 |
| Mt-Co2 | /5Phos/GTGAACTACGACTGCTAGAA-TCCCTTTAGTGAGGGTTAAT | SEQ ID NO: 146 |
| Mt-Co3 | /5Phos/CTCCAAGCTTCAGAATACTT-TCCCTTTAGTGAGGGTTAAT | SEQ ID NO: 147 |
| Mt-Cytb | /5Phos/CTCAAAGCAACGAAGCCTAA-TCCCTTTAGTGAGGGTTAAT | SEQ ID NO: 148 |
| Mt-Nd1 | /5Phos/CTATGGATCCGAGCATCTTA-TCCCTTTAGTGAGGGTTAAT | SEQ ID NO: 149 |
| Mt-Nd2 | /5Phos/CTCACTCTACTCAACCTCAT-TCCCTTTAGTGAGGGTTAAT | SEQ ID NO: 150 |
| Mt-Nd3 | /5Phos/CTAGAAATTGCTCTTCTACT-TCCCTTTAGTGAGGGTTAAT | SEQ ID NO: 151 |
| Mt-Nd4 | /5Phos/CGTACTATAATCATGGCCGG-TCCCTTTAGTGAGGGTTAAT | SEQ ID NO: 152 |
| Mt-Nd4I | /5Phos/GGTAAACTCCAACTCCATAA-TCCCTTTAGTGAGGGTTAAT | SEQ ID NO: 153 |
| Mt-Nd5 | /5Phos/CCACTTCTATAACAGCTATG-TCCCTTTAGTGAGGGTTAAT | SEQ ID NO: 153 |
| Mt-Nd6 | /5Phos/GTTGATGATGTTGGAGTTAT-TGCCTTTAGTGAGGGTTAAT | SEQ ID NO: 154 |
| Atp5a1 | /5Phos/CTGATGTACAGAAATCACAT-TCCCTTTAGTGAGGGTTAAT | SEQ ID NO: 155 |
| Atp5c1 | /5Phos/GCCAGGCTGTCATCACAAAG-TCCCTTTAGTGAGGGTTAAT | SEQ ID NO: 156 |
| Atp5o | /5Phos/CAAACCAAATACTGAAACTG-TCCCTTTAGTGAGGGTTAAT | SEQ ID NO: 157 |
| Cox5b | /5Phos/CAAGGAAGACCCTAATCTAG-TCCCTTTAGTGAGGGTTAAT | SEQ ID NO: 158 |
| | Upstream primer full sequence (5′-3′) | |
| | [T7sequence-Tag-UpstreamTargetSequence] | |
| Cox7a2 | /5Phos/CATTTTGTGTCTCCCTTTTC-TCCCTTTAGTGAGGGTTAAT | SEQ ID NO: 159 |
| Cyc1 | /5Phos/CATGGCTCCTCCCATCTACA-TCCCTTTAGTGAGGGTTAAT | SEQ ID NO: 160 |
| Hspc051 | /5Phos/CCGTTCACCGACCGCCAGTG-TCCCTTTAGTGAGGGTTAAT | SEQ ID NO: 161 |
| Ndufa5 | /5Phos/CTAAACATGCAGCCTATAGA-TCCCTTTAGTGAGGGTTAAT | SEQ ID NO: 162 |
| Ndufb5 | /5Phos/CCTCTAGTGGGAAGAAATGATCCCTTTAGTGAGGGTTAAT | SEQ ID NO: 163 |
| Sdhd | /5Phos/GGAGTTCTCCTTGTTTGTGGTCCCTTTAGTGAGGGTTAAT | SEQ ID NO: 164 |
| Uqcrb | /5Phos/GTATTATAATGCTGCAGGATTCCCTTTAGTGAGGGTTAAT | SEQ ID NO: 165 |
| Uqrc1 | /5Phos/CGCAGTGGCATGTTCTGGCTTCCCTTTAGTGAGGGTTAAT | SEQ ID NO: 166 |
| Actb | /5Phos/CTCACCCTCCCAAAAGCCACTCCCTTTAGTGAGGGTTAAT | SEQ ID NO: 167 |
| Aamp | /5Phos/GGGTTAGGTCTTTGAGGTTCTCCCTTTAGTGAGGGTTAAT | SEQ ID NO: 168 |
| Cenpb | /5Phos/CTTTCCCAGCTTGAATTCAATCCCTTTAGTGAGGGTTAAT | SEQ ID NO: 169 |
| Eef1a1 | /5Phos/CTTCAGAAGGAAAGAATGTTTCCCTTTAGTGAGGGTTAAT | SEQ ID NO: 170 |
| Jund | /5Phos/CTGCGCGTGGCTGCCCCTTTTCCCTTTAGTGAGGGTTAAT | SEQ ID NO: 171 |
| Lsp1 | /5Phos/CTTCTCACCATCAGCTAAAGTCCCTTTAGTGAGGGTTAAT | SEQ ID NO: 172 |
| Rps2 | /5Phos/CCACACCAGAGTCTCTGTTCTCCCTTTAGTGAGGGTTAAT | SEQ ID NO: 173 |
| Rps27a | /5Phos/GCCGGACTTTGTCTGACTACTCCCTTTAGTGAGGGTTAAT | SEQ ID NO: 174 |
| Cyb5r3 | /5Phos/CCCTAAGTTGTCAGCCCAACTCCCTTTAGTGAGGGTTAAT | SEQ ID NO: 175 |
| Fhl1 | /5Phos/CCTCCTTAACTGTACTCTCCTCCCTTTAGTGAGGGTTAAT | SEQ ID NO: 176 |

We claim:

1. A method of treating or preventing a disorder characterized by mitochondrial dysfunction in a subject, the method comprising administering to the subject a therapeutically effective amount of a cytoskeleton modulator.

2. The method of claim 1, wherein the cytoskeleton modulator is a microtubule modulator.

3. The method of claim 2, wherein the microtubule modulator is a microtubule inhibitor.

4. The method of claim 1, wherein the cytoskeleton modulator is a compound of Formula (I): 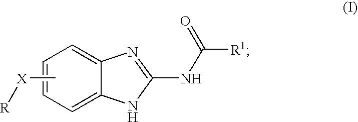
wherein R is selected from (C₁-C₄)alkyl, cycloalkyl having 3 to 6 carbon atoms, phenyl, halo-substituted phenyl in which halo in each occurrence is selected from Br, Cl, or F, (lower alkyl)-substituted phenyl, ((C₁-C₄)alkoxy)-substituted phenyl, and 2-thienyl; R¹ is selected from methyl and ethyl, X is selected from —S—, —C(O)—, —O—, —CH₂— and —S(O)— and the R—X— substituent is located at the 5(6)-position, or a salt thereof.

5. The method of claim 4, wherein the compound is mebendazole, a derivative, metabolite, or analog thereof.

6. The method of claim 5, wherein the subject is not afflicted with a worm infection.

7. The method of claim 5, wherein the subject is not afflicted with diabetes.

8. The method of claim 4, wherein the compound is nocodazole, a derivative, metabolite, or analog thereof.

9. The method of claim 4, wherein the compound is one of the following: albendazole, fenbendazole, oxfendazole, oxibendazole, methiazole, parbendazole, and any derivatives, metabolites, or analogs of the compounds listed.

10. The method of claim 1, wherein the cytoskeleton modulator is cytochalasin, a derivative, metabolite, or analog thereof.

11. The method of claim 10, wherein the cytochalasin is selected from cytochalasin A, cytochalasin B, cytochalasin C, cytochalasin D, cytochalasin E, cytochalasin F, cytochalasin H, cytochalasin J, cytochalasin K, cytochalasin Q, cytochalasin R, epoxycytochalasin H and epoxycytochalasin J.

12. The method of claim 11, wherein the cytochalasin is selected from cytochalasin E.

13. The method of claim 1, wherein the cytoskeleton modulator is a compound of Formula (II): 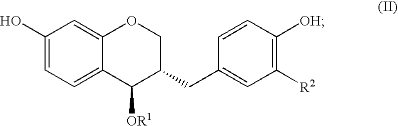
wherein R¹ is selected from H or methyl and R² is selected from H or hydroxy.

14. The method of claim 1, wherein the cytoskeleton modulator is a compound selected from Formulas (III)-(VI):

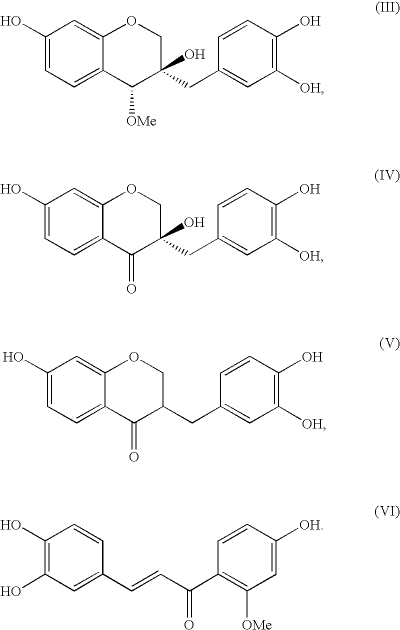

15. The method of claim 14, wherein the compound is deoxysappanone B, or a metabolite, or an analog thereof.

16. The method of claim 15, wherein the deoxysappanone is selected from deoxysappanone (B) 7,3′-dimethyl ether, sappanone (A) trimethyl ether, or 3-deshydroxysappanol trimethyl ether.

17. The method of claim 15, wherein the subject is not afflicted with diabetes.

18. The method of claim 1, wherein the cytoskeleton modulator is a compound of Formula (VII): 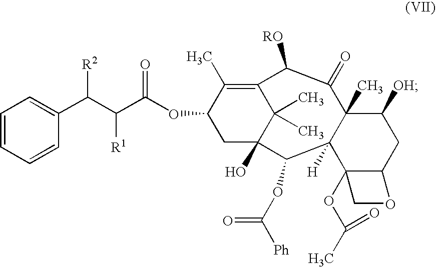
wherein, R is nitrogen or acetyl and one of R¹ and R² is hydroxy and the other is selected from t-butylcarbonylamino or benzoylamino.

19. The method of claim 18, wherein the compound is paclitaxel or a metabolite or analog thereof.

20. The method of claim 1, wherein the compound is podofilox, a metabolite, analog, or salt thereof.

21. The method of claim 20, wherein the compound is podophyllotoxin acetate.

22. The method of claim 1, wherein the cytoskeleton modulator is a compound of Formula (VIII): 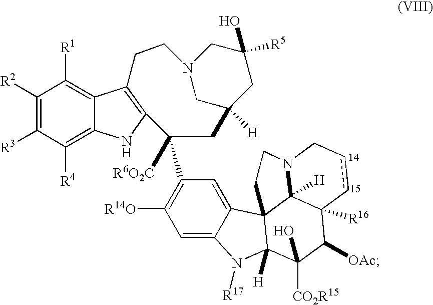
wherein R¹, R², R³ and R⁴ are independently selected from H, lower alkyl group, lower alkoxy group, halogen, lower perfluoroalkyl group, lower alkylthio group, hydroxy group, amino group, mono- or di-alkyl or acylamino group, lower alkyl or arylsulfonyloxy group, R⁵ is H, or a lower alkyl group or a substituted or non-substituted aryl group, R⁶ is an alkyl group of carbon number 4 or less, R¹⁴, R¹⁵ and R¹⁶ are an alkyl group of carbon number 4 or less, R¹⁷ is H or an alkyl group of carbon number 4 or less, and in between carbon 14 and carbon 15 is an unsaturated double bond or saturated bond.

23. The method of claim 22, wherein the compound is vinblastine or a metabolite or analog thereof.

24. The method of claim 1, wherein the mitochondrial dysfunction is characterized by reduced oxidative phosphorylation or increased generation of reactive oxygen species or both.

25. The method of claim 1, wherein the disorder is, obesity, cardiac myopathy, premature aging, coronary atherosclerotic heart disease, diabetes mellitus, Alzheimer's Disease, Parkinson's Disease, Huntington's disease, dystonia, Leber's hereditary optic neuropathy (LHON), schizophrenia, myodegenerative disorders such as “mitochondrial encephalopathy, lactic acidosis, and stroke” (MELAS) and “myoclonic epilepsy ragged red fiber syndrome” (MERRF), NARP (Neuropathy; Ataxia; Retinitis Pigmentosa), MNGIE (Myopathy and external opthalmoplegia, neuropathy; gastro-intestinal encephalopathy, Kearns-Sayre disease, Pearson's Syndrome, PEO (Progressive External Opthalmoplegia), congenital muscular dystrophy with mitochondrial structural abnormalities, Wolfram syndrome, Diabetes Insipidus, Diabetes Mellitus, Optic Atrophy Deafness, Leigh's Syndrome, fatal infantile myopathy with severe mitochondrial DNA (mtDNA) depletion, benign “later-onset” myopathy with moderate reduction in mtDNA, dystonia, medium chain acyl-CoA dehydrogenase deficiency, arthritis, and maternally inherited diabetes with deafness (MIDD), mitochondrial DNA depletion syndrome.

26. The method of claim 1, wherein the subject is not afflicted with cancer.

27. The method of claim 1, wherein the disorder is obesity.

28. The method of claim 1, wherein the disorder is diabetes.

29. The method of claim 28, wherein the diabetes is type 2 diabetes mellitus.

30. The method of claim 1, wherein the disorder is glucose intolerance.

31. The method of claim 1, wherein the subject has elevated gluconeogenesis.

32. The method of claim 1, wherein the disorder is premature aging.

33. The method of claim 1, wherein the disorder is a neurodegenerative disorder.

34. The method of claim 1, wherein the disorder is an mtDNA-associated disease.

35. The method of claim 1, wherein the disorder is a mitochondrial encephalomyopathy due to nuclear gene mutations.

36. The method of claim 1, wherein the disorder is a congenital mitochondrial disorder.

37. The method of claim 1, wherein the disorder is cardiovascular disease.

38. The method of claim 1, wherein the disorder is cardiomyopathy.

39. The method of claim 1, further comprising administering to the subject one or more agents selected from sulfonylureas, non-sulfonylurea secretagogues, insulin, insulin analogs, glucagon-like peptides, exendin-4 polypeptides, beta 3 adrenoceptor agonists, PPAR agonists, dipeptidyl peptidase IV inhibitors, biguanides, alpha-glucosidase inhibitors, immunomodulators, statins and statin-containing combinations, angiotensin converting enzyme inhibitors, adeno sine A1 receptor agonists, adenosine A2 receptor agonists, aldosterone antagonists, alpha 1 adrenoceptor antagonists, alpha 2 adrenoceptor agonists, alpha 2 adrenoceptor agonists, angiotensin receptor antagonists, antioxidants, ATPase inhibitors, atrial peptide agonists, beta adrenoceptor antagonists, calcium channel agonists, calcium channel antagonists, diuretics, dopamine D1 receptor agonists, endopeptidase inhibitors, endothelin receptor antagonists, guanylate cyclase stimulants, phosphodiesterase V inhibitors, protein kinase inhibitors, Cdc2 kinase inhibitors, renin inhibitors, thromboxane synthase inhibitors, vasopeptidase inhibitors, vasopressin I antagonists, vasopressin 2 antagonists, angiogenesis inhibitors, advanced glycation end product inhibitors, bile acid binding agents, bile acid transport inhibitors, bone formation stimulants, apolipoprotein A1 agonists, DNA topoisomerase inhibitors, cholesterol absorption inhibitors, cholesterol antagonists, cholesteryl ester transfer protein antagonists, cytokine synthesis inhibitors, DNA polymerase inhibitors, dopamine D2 receptor agonists, endothelin receptor antagonists, growth hormone antagonists, insulin sensitizers, lipase inhibitors, lipid peroxidation inhibitors, lipoprotein A antagonists, microsomal transport protein inhibitors, microsomal triglyceride transfer protein inhibitors, nitric oxide synthase inhibitors, oxidizing agents, phospholipase A2 inhibitors, radical formation agonists, platelet aggregation antagonists, prostaglandin synthase stimulants, reverse cholesterol transport activators, rho kinase inhibitors, selective estrogen receptor modulators, squalene epoxidase inhibitors, squalene synthase inhibitors, thromboxane A2 antagonists, amylin agonists, cannabinoid receptor antagonists, cholecystokinin A agonists, corticotropin-releasing factor agonists, dopamine uptake inhibitors, G protein-coupled receptor modulators, glutamate antagonists, glucagon-like peptide-1 agonists, insulin sensitizers, lipase inhibitors, melanin-concentrating hormone receptor antagonists, nerve growth factor agonists, neuropeptide Y agonists, neuropeptide Y antagonists, SNRIs, protein tyrosine phosphatase inhibitors, serotonin 2C receptor agonists, bezafibrate, diflunisal, or cinnamic acid.

40. A method for identifying compounds that enhance mitochondrial function comprising (i) assaying for the effect of one or more compounds on (a) OXPHOS gene expression and (b) mitochondrial function; and (ii) correlating the effect with a compound's enhancement of mitochondrial function, wherein an increase in OXPHOS gene expression and an increase in mitochondrial function is indicative of a compound that enhances mitochondrial function.

41. A method for identifying compounds for treating a disorder characterized by mitochondrial dysfunction in a subject comprising (i) assaying for the effect of one or more compounds on (a) OXPHOS gene expression and (b) mitochondrial function; and (ii) correlating the effect with a compound's ability to treat said disorder, wherein an increase in OXPHOS gene expression and an increase in mitochondrial function is indicative of a compound useful for treating said disorder.

42. A method for determining compounds that are contraindicated in a subject, comprising (i) assaying for the effect of one or more compounds on (a) cellular dehydrogenase activity and (b) cell viability; and (ii) correlating the effect with contraindication of a compound, wherein a decrease in cellular dehydrogenase activity absent a decrease in cell viability indicates that the compound is contraindicated for said subjects.

43. A method for determining two or more compounds that are contraindicated for joint administration to a subject comprising (i) assaying for the effect of two or more compounds on (a) cellular dehydrogenase activity and (b) cell viability; and (ii) correlating the effect with contraindication of joint administration, wherein two or more compounds that each decrease cellular dehydrogenase activity absent a decrease in cell viability indicates that the two or more compounds are contraindicated when jointly administered to a subject.

44. A kit comprising a plurality of primer pairs wherein each primer pair comprises a first nucleic acid sequence and a second nucleic acid sequence which first nucleic acid sequence hybridizes under stringent conditions to a first strand of a target sequence, and which second nucleic acid sequence hybridizes under stringent conditions to a second strand of a target sequence, wherein the target sequence is selected from a group consisting of the following: (a) Mt-Atp6, (b) Mt-Atp8, (c) Mt-Co1, (d) Mt-Co2, (e) Mt-Co3, (f) Mt-Cytb, (g) Mt-Nd1, (h) Mt-Nd2, (i) Mt-Nd3, (j) Mt-Nd4, (k) Mt-Nd41, (l) Mt-Nd5, (m) Mt-Nd61, (n) Atp5a1, (o) Atp5c1, (p) Atp5o, (q) Cox5b, (r) Cox7a2, (s) Cyc1, (t) Hspc051, (u) Ndufa5, (v) Ndufb5, (w) Sdhd, (x) Uqcrb, and (y) Uqcrc1.

45. The kit of claim 44, wherein each first nucleic acid and/or the second nucleic acid further comprises a tag sequence.

46. The kit of claim 45, wherein said tag sequence does not hybridize to the target sequence.

47. The kit of claim 45, wherein said tag sequence is selected from the following: (a) SEQ ID NO:71, (b) SEQ ID NO:72, (c) SEQ ID NO:73, (d) SEQ ID NO:74, (e) SEQ ID NO:75, (f) SEQ ID NO:76, (g) SEQ ID NO:77, (h) SEQ ID NO:78, (i) SEQ ID NO:79, (j) SEQ ID NO:80, (k) SEQ ID NO:81, (l) SEQ ID NO:82, (m) SEQ ID NO:83, (n) SEQ ID NO:84, (o) SEQ ID NO:85, (p) SEQ ID NO:86, (q) SEQ ID NO:87, (r) SEQ ID NO:88, (s) SEQ ID NO:89, (t) SEQ ID NO:90, (u) SEQ ID NO:91, (v) SEQ ID NO:92, (w) SEQ ID NO:93, (x) SEQ ID NO:94, (y) SEQ ID NO:95, (z) SEQ ID NO:96, (aa) SEQ ID NO:97, (bb) SEQ ID NO:98, (cc) SEQ ID NO:99, (dd) SEQ ID NO:100, (ee) SEQ ID NO:101, (ff) SEQ ID NO:102, (gg) SEQ ID NO:103, (hh) SEQ ID NO:104, (ii) SEQ ID NO:105.

48. A method of detecting levels of at least 2 OXPHOS genes, comprising:
(1) providing one or more target sequences selected from the following: (a) Mt-Atp6, (b) Mt-Atp8, (c) Mt-Co1, (d) Mt-Co2, (e) Mt-Co3, (f) Mt-Cytb, (g) Mt-Nd1, (h) Mt-Nd2, (i) Mt-Nd3, (j) Mt-Nd4, (k) Mt-Nd41, (l) Mt-Nd5, (m) Mt-Nd61, (n) Atp5a1, (o) Atp5c1, (p) Atp5o, (q) Cox5b, (r) Cox7a2, (s) Cyc1, (t) Hspc051, (u) Ndufa5, (v) Ndufb5, (w) Sdhd, (x) Uqcrb, and (y) Uqcrc1,
(2) providing the plurality of primers that hybridize under stringent conditions to a target sequence from step (1)
(3) amplifying target sequences using primers,
(4) amplifying the sequences of step (3) using 2 nucleic acid sequences that are complementary to at least 1 portion of the primers of step (2), wherein one nucleic acid sequence is linked to a binding moiety, and one nucleic acid sequence is phosphorylated,
(5) identifying the amplification products of step (4) by hybridization to a nucleic acid sequence that is complementary to a portion of the amplification product, wherein nucleic acid sequence is covalently linked to a detectable moiety.

49. The method of claim 48, wherein said amplification products are quantified by binding a second detectable moiety to said binding moiety.

50. The method of claim 51, wherein said binding moiety is biotin and said second binding moiety is avidin or streptavidin.

51. The method of claim 51, wherein said detectable moiety is a microsphere.

52. The method of claim 51, wherein steps (1)-(4) are performed in a microtiter plate.


SEQUENCE LISTING (177 sequences)

SEQ ID NO: 1 - DNA, 20 bp, Artificial Sequence
  Description of Artificial Sequence Synthetic probe
ttcaagcctacgtattcacc

SEQ ID NO: 2 - DNA, 20 bp, Artificial Sequence
  Description of Artificial Sequence Synthetic probe
ctcctagtaagcctatatct

SEQ ID NO: 3 - DNA, 20 bp, Artificial Sequence
  Description of Artificial Sequence Synthetic probe
tcaccaaaatcactaacaac

SEQ ID NO: 4 - DNA, 20 bp, Artificial Sequence
  Description of Artificial Sequence Synthetic probe
cataaaagtaaaaacccctt

SEQ ID NO: 5 - DNA, 20 bp, Artificial Sequence
  Description of Artificial Sequence Synthetic probe
cacgacgctactcagactac

SEQ ID NO: 6 - DNA, 20 bp, Artificial Sequence
  Description of Artificial Sequence Synthetic probe
ccagatgcttacaccacatg

SEQ ID NO: 7 - DNA, 20 bp, Artificial Sequence
  Description of Artificial Sequence Synthetic probe
aacaaacgacctaaaacctg

SEQ ID NO: 8 - DNA, 20 bp, Artificial Sequence
  Description of Artificial Sequence Synthetic probe
gtgaactacgactgctagaa

SEQ ID NO: 9 - DNA, 20 bp, Artificial Sequence
  Description of Artificial Sequence Synthetic probe
taggactttacttcaccatc

SEQ ID NO: 10 - DNA, 20 bp, Artificial Sequence
  Description of Artificial Sequence Synthetic probe
ctccaagcttcagaatactt

SEQ ID NO: 11 - DNA, 20 bp, Artificial Sequence
  Description of Artificial Sequence Synthetic probe
ctaatacctttccttcatac

SEQ ID NO: 12 - DNA, 20 bp, Artificial Sequence
  Description of Artificial Sequence Synthetic probe
ctcaaagcaacgaagcctaa

SEQ ID NO: 13 - DNA, 20 bp, Artificial Sequence
  Description of Artificial Sequence Synthetic probe
tactactatcatcaacattc

SEQ ID NO: 14 - DNA, 20 bp, Artificial Sequence
  Description of Artificial Sequence Synthetic probe
ctatggatccgagcatctta

SEQ ID NO: 15 - DNA, 20 bp, Artificial Sequence
  Description of Artificial Sequence Synthetic probe
ttcttccttacaacccatcc

SEQ ID NO: 16 - DNA, 20 bp, Artificial Sequence
  Description of Artificial Sequence Synthetic probe
ctcactctactcaacctcat

SEQ ID NO: 17 - DNA, 20 bp, Artificial Sequence
  Description of Artificial Sequence Synthetic probe
ttacatttctattatttgac

SEQ ID NO: 18 - DNA, 20 bp, Artificial Sequence
  Description of Artificial Sequence Synthetic probe
ctagaaattgctcttctact

SEQ ID NO: 19 - DNA, 20 bp, Artificial Sequence
  Description of Artificial Sequence Synthetic probe
actacgaacggatccacagc

SEQ ID NO: 20 - DNA, 20 bp, Artificial Sequence
  Description of Artificial Sequence Synthetic probe
cgtactataatcatggcccg

SEQ ID NO: 21 - DNA, 20 bp, Artificial Sequence
  Description of Artificial Sequence Synthetic probe
attataacttcagtaacttc

SEQ ID NO: 22 - DNA, 20 bp, Artificial Sequence
  Description of Artificial Sequence Synthetic probe
cctaaactccaactccataa

SEQ ID NO: 23 - DNA, 20 bp, Artificial Sequence
  Description of Artificial Sequence Synthetic probe
cctactaattacactaatcg

SEQ ID NO: 24 - DNA, 20 bp, Artificial Sequence
  Description of Artificial Sequence Synthetic probe
ccacttctataacagctatg

SEQ ID NO: 25 - DNA, 20 bp, Artificial Sequence
  Description of Artificial Sequence Synthetic probe
gagattggttgatgtatgag

SEQ ID NO: 26 - DNA, 20 bp, Artificial Sequence
  Description of Artificial Sequence Synthetic probe
gttgatgatgttggagttat

SEQ ID NO: 27 - DNA, 20 bp, Artificial Sequence
  Description of Artificial Sequence Synthetic probe
aaagggttactcttgtattc

SEQ ID NO: 28 - DNA, 20 bp, Artificial Sequence
  Description of Artificial Sequence Synthetic probe
ctgatgtacagaaatcacat

SEQ ID NO: 29 - DNA, 20 bp, Artificial Sequence
  Description of Artificial Sequence Synthetic probe
cttgactttcaaccgcaccc

SEQ ID NO: 30 - DNA, 20 bp, Artificial Sequence
  Description of Artificial Sequence Synthetic probe
gccaggctgtcatcacaaag

SEQ ID NO: 31 - DNA, 20 bp, Artificial Sequence
  Description of Artificial Sequence Synthetic probe
gctgaagagcttcctgagtc

SEQ ID NO: 32 - DNA, 20 bp, Artificial Sequence
  Description of Artificial Sequence Synthetic probe
caaaccaaatactgaaactg

SEQ ID NO: 33 - DNA, 20 bp, Artificial Sequence
  Description of Artificial Sequence Synthetic probe
ccaaaggcagcttcaggcac

SEQ ID NO: 34 - DNA, 20 bp, Artificial Sequence
  Description of Artificial Sequence Synthetic probe
caaggaagaccctaatctag

SEQ ID NO: 35 - DNA, 20 bp, Artificial Sequence
  Description of Artificial Sequence Synthetic probe
ccaataaagcaatccttaac

SEQ ID NO: 36 - DNA, 20 bp, Artificial Sequence
  Description of Artificial Sequence Synthetic probe
cattttgtgtctcccttttc

SEQ ID NO: 37 - DNA, 20 bp, Artificial Sequence
  Description of Artificial Sequence Synthetic probe
tttcccggccaggccattgg

SEQ ID NO: 38 - DNA, 20 bp, Artificial Sequence
  Description of Artificial Sequence Synthetic probe
catggctcctcccatctaca

SEQ ID NO: 39 - DNA, 20 bp, Artificial Sequence
  Description of Artificial Sequence Synthetic probe
taaggatgagtttcaagttg

SEQ ID NO: 40 - DNA, 20 bp, Artificial Sequence
  Description of Artificial Sequence Synthetic probe
ccgttcaccgaccgccagtg

SEQ ID NO: 41 - DNA, 20 bp, Artificial Sequence
  Description of Artificial Sequence Synthetic probe
tgatattctgaagcactttc

SEQ ID NO: 42 - DNA, 20 bp, Artificial Sequence
  Description of Artificial Sequence Synthetic probe
ctaaacatgcagcctataga

SEQ ID NO: 43 - DNA, 20 bp, Artificial Sequence
  Description of Artificial Sequence Synthetic probe
ctgtccaagaacagtgtgtc

SEQ ID NO: 44 - DNA, 20 bp, Artificial Sequence
  Description of Artificial Sequence Synthetic probe
cctctagtgggaagaaatga

SEQ ID NO: 45 - DNA, 20 bp, Artificial Sequence
  Description of Artificial Sequence Synthetic probe
tttagacaagttcaatttag

SEQ ID NO: 46 - DNA, 20 bp, Artificial Sequence
  Description of Artificial Sequence Synthetic probe
ggagttctccttgtttgtgg

SEQ ID NO: 47 - DNA, 20 bp, Artificial Sequence
  Description of Artificial Sequence Synthetic probe
ctggatggttttcgaaagtg

SEQ ID NO: 48 - DNA, 20 bp, Artificial Sequence
  Description of Artificial Sequence Synthetic probe
gtattataatgctgcaggat

SEQ ID NO: 49 - DNA, 20 bp, Artificial Sequence
  Description of Artificial Sequence Synthetic probe
tcccagactacaaccggatc

SEQ ID NO: 50 - DNA, 20 bp, Artificial Sequence
  Description of Artificial Sequence Synthetic probe
cgcagtggcatgttctggct

SEQ ID NO: 51 - DNA, 20 bp, Artificial Sequence
  Description of Artificial Sequence Synthetic probe
taagtggttacaggaagtcc

SEQ ID NO: 52 - DNA, 20 bp, Artificial Sequence
  Description of Artificial Sequence Synthetic probe
ctcaccctcccaaaagccac

SEQ ID NO: 53 - DNA, 20 bp, Artificial Sequence
  Description of Artificial Sequence Synthetic probe
gggtgcgtctttgtatgttg

SEQ ID NO: 54 - DNA, 20 bp, Artificial Sequence
  Description of Artificial Sequence Synthetic probe
gggttaggtctttgaggttc

SEQ ID NO: 55 - DNA, 20 bp, Artificial Sequence
  Description of Artificial Sequence Synthetic probe
gtccagccacccaggtgctc

SEQ ID NO: 56 - DNA, 20 bp, Artificial Sequence
  Description of Artificial Sequence Synthetic probe
ctttcccagcttgaattcaa

SEQ ID NO: 57 - DNA, 20 bp, Artificial Sequence
  Description of Artificial Sequence Synthetic probe
ataacaatgcatcgtaaaac

SEQ ID NO: 58 - DNA, 20 bp, Artificial Sequence
  Description of Artificial Sequence Synthetic probe
cttcagaaggaaagaatgtt

SEQ ID NO: 59 - DNA, 20 bp, Artificial Sequence
  Description of Artificial Sequence Synthetic probe
ccgcctctctacccccagtc

SEQ ID NO: 60 - DNA, 20 bp, Artificial Sequence
  Description of Artificial Sequence Synthetic probe
ctgcgcgtggctgccccttt

SEQ ID NO: 61 - DNA, 20 bp, Artificial Sequence
  Description of Artificial Sequence Synthetic probe
tgaccaaccctccaactctg

SEQ ID NO: 62 - DNA, 20 bp, Artificial Sequence
  Description of Artificial Sequence Synthetic probe
cttctcaccatcagctaaag

SEQ ID NO: 63 - DNA, 20 bp, Artificial Sequence
  Description of Artificial Sequence Synthetic probe
acggatcatcttgtgaaaac

SEQ ID NO: 64 - DNA, 20 bp, Artificial Sequence
  Description of Artificial Sequence Synthetic probe
ccacaccagagtctctgttc

SEQ ID NO: 65 - DNA, 20 bp, Artificial Sequence
  Description of Artificial Sequence Synthetic probe
tggtaagcagctggaagatg

SEQ ID NO: 66 - DNA, 20 bp, Artificial Sequence
  Description of Artificial Sequence Synthetic probe
gccggactttgtctgactac

SEQ ID NO: 67 - DNA, 20 bp, Artificial Sequence
  Description of Artificial Sequence Synthetic probe
actccatgcagtcttgagtg

SEQ ID NO: 68 - DNA, 20 bp, Artificial Sequence
  Description of Artificial Sequence Synthetic probe
ccctaagttgtcagcccaac

SEQ ID NO: 69 - DNA, 20 bp, Artificial Sequence
  Description of Artificial Sequence Synthetic probe
ttctctgaaacgcaggattg

SEQ ID NO: 70 - DNA, 20 bp, Artificial Sequence
  Description of Artificial Sequence Synthetic probe
cctccttaactgtactctcc

SEQ ID NO: 71 - DNA, 24 bp, Artificial Sequence
  Description of Artificial Sequence Synthetic oligonucleotide
ctttaatctcaatcaatacaaatc

SEQ ID NO: 72 - DNA, 24 bp, Artificial Sequence
  Description of Artificial Sequence Synthetic oligonucleotide
ctttatcaatacatactacaatca

SEQ ID NO: 73 - DNA, 24 bp, Artificial Sequence
  Description of Artificial Sequence Synthetic oligonucleotide
tacactttatcaaatcttacaatc

SEQ ID NO: 74 - DNA, 24 bp, Artificial Sequence
  Description of Artificial Sequence Synthetic oligonucleotide
tacattaccaataatcttcaaatc

SEQ ID NO: 75 - DNA, 24 bp, Artificial Sequence
  Description of Artificial Sequence Synthetic oligonucleotide
tcaacaatcttttacaatcaaatc

SEQ ID NO: 76 - DNA, 24 bp, Artificial Sequence
  Description of Artificial Sequence Synthetic oligonucleotide
caattcatttaccaatttaccaat

SEQ ID NO: 77 - DNA, 24 bp, Artificial Sequence
  Description of Artificial Sequence Synthetic oligonucleotide
aatccttttacattcattacttac

SEQ ID NO: 78 - DNA, 24 bp, Artificial Sequence
  Description of Artificial Sequence Synthetic oligonucleotide
taatcttctatatcaacatcttac

SEQ ID NO: 79 - DNA, 24 bp, Artificial Sequence
  Description of Artificial Sequence Synthetic oligonucleotide
atcatacatacatacaaatctaca

SEQ ID NO: 80 - DNA, 24 bp, Artificial Sequence
  Description of Artificial Sequence Synthetic oligonucleotide
tacaaatcatcaatcactttaatc

SEQ ID NO: 81 - DNA, 24 bp, Artificial Sequence
  Description of Artificial Sequence Synthetic oligonucleotide
atacttcattcattcatcaattca

SEQ ID NO: 82 - DNA, 24 bp, Artificial Sequence
  Description of Artificial Sequence Synthetic oligonucleotide
tcaaaatctcaaatactcaaatca

SEQ ID NO: 83 - DNA, 24 bp, Artificial Sequence
  Description of Artificial Sequence Synthetic oligonucleotide
tcaatcaattacttactcaaatac

SEQ ID NO: 84 - DNA, 24 bp, Artificial Sequence
  Description of Artificial Sequence Synthetic oligonucleotide
ttcacttttcaatcaactttaatc

SEQ ID NO: 85 - DNA, 24 bp, Artificial Sequence
  Description of Artificial Sequence Synthetic oligonucleotide
attattcacttcaaactaatctac

SEQ ID NO: 86 - DNA, 24 bp, Artificial Sequence
  Description of Artificial Sequence Synthetic oligonucleotide
tcaattacttcactttaatccttt

SEQ ID NO: 87 - DNA, 24 bp, Artificial Sequence
  Description of Artificial Sequence Synthetic oligonucleotide
tcattcatatacataccaattcat

SEQ ID NO: 88 - DNA, 24 bp, Artificial Sequence
  Description of Artificial Sequence Synthetic oligonucleotide
caatttcatcattcattcatttca

SEQ ID NO: 89 - DNA, 24 bp, Artificial Sequence
  Description of Artificial Sequence Synthetic oligonucleotide
caattcatttcattcacaatcaat

SEQ ID NO: 90 - DNA, 24 bp, Artificial Sequence
  Description of Artificial Sequence Synthetic oligonucleotide
cttttcatcttttcatctttcaat

SEQ ID NO: 91 - DNA, 24 bp, Artificial Sequence
  Description of Artificial Sequence Synthetic oligonucleotide
tcaatcattacacttttcaacaat

SEQ ID NO: 92 - DNA, 24 bp, Artificial Sequence
  Description of Artificial Sequence Synthetic oligonucleotide
ctttctacattattcacaacatta

SEQ ID NO: 93 - DNA, 24 bp, Artificial Sequence
  Description of Artificial Sequence Synthetic oligonucleotide
ctatcttcatatttcactataaac

SEQ ID NO: 94 - DNA, 24 bp, Artificial Sequence
  Description of Artificial Sequence Synthetic oligonucleotide
ctttcaattacaatactcattaca

SEQ ID NO: 95 - DNA, 24 bp, Artificial Sequence
  Description of Artificial Sequence Synthetic oligonucleotide
tcatttaccaatctttctttatac

SEQ ID NO: 96 - DNA, 24 bp, Artificial Sequence
  Description of Artificial Sequence Synthetic oligonucleotide
tcatttcacaattcaattactcaa

SEQ ID NO: 97 - DNA, 24 bp, Artificial Sequence
  Description of Artificial Sequence Synthetic oligonucleotide
tacatcaacaattcattcaataca

SEQ ID NO: 98 - DNA, 24 bp, Artificial Sequence
  Description of Artificial Sequence Synthetic oligonucleotide
cttctcattaacttacttcataat

SEQ ID NO: 99 - DNA, 24 bp, Artificial Sequence
  Description of Artificial Sequence Synthetic oligonucleotide
aaacaaacttcacatctcaataat

SEQ ID NO: 100 - DNA, 24 bp, Artificial Sequence
  Description of Artificial Sequence Synthetic oligonucleotide
tcatcaatctttcaatttacttac

SEQ ID NO: 101 - DNA, 24 bp, Artificial Sequence
  Description of Artificial Sequence Synthetic oligonucleotide
caatataccaatatcatcatttac

SEQ ID NO: 102 - DNA, 24 bp, Artificial Sequence
  Description of Artificial Sequence Synthetic oligonucleotide
tcatttcaatcaatcatcaacaat

SEQ ID NO: 103 - DNA, 24 bp, Artificial Sequence
  Description of Artificial Sequence Synthetic oligonucleotide
tcaatcatctttatacttcacaat

SEQ ID NO: 104 - DNA, 24 bp, Artificial Sequence
  Description of Artificial Sequence Synthetic oligonucleotide
ctacatattcaaattactacttac

SEQ ID NO: 105 - DNA, 24 bp, Artificial Sequence
  Description of Artificial Sequence Synthetic oligonucleotide
cttttcatcaataatcttaccttt

SEQ ID NO: 106 - DNA, 20 bp, Artificial Sequence
  Description of Artificial Sequence Synthetic primer
taatacgactcactataggg

SEQ ID NO: 107 - DNA, 20 bp, Artificial Sequence
  Description of Artificial Sequence Synthetic primer
tccctttagtgagggttaat

SEQ ID NO: 108 - DNA, 64 bp, Artificial Sequence
  Description of Artificial Sequence Synthetic probe
taatacgactcactatagggcaattcatttaccaatttaccaatttcaagcctacgtattcacc

SEQ ID NO: 109 - DNA, 64 bp, Artificial Sequence
  Description of Artificial Sequence Synthetic probe
taatacgactcactatagggtcaatcattacacttttcaacaattcaccaaaatcactaacaac

SEQ ID NO: 110 - DNA, 64 bp, Artificial Sequence
  Description of Artificial Sequence Synthetic probe
taatacgactcactatagggaatccttttacattcattacttaccacgacgctactcagactac

SEQ ID NO: 111 - DNA, 64 bp, Artificial Sequence
  Description of Artificial Sequence Synthetic probe
taatacgactcactatagggctttctacattattcacaacattaaacaaacgacctaaaacctg

SEQ ID NO: 112 - DNA, 64 bp, Artificial Sequence
  Description of Artificial Sequence Synthetic probe
taatacgactcactatagggctatcttcatatttcactataaactaggactttacttcaccatc

SEQ ID NO: 113 - DNA, 64 bp, Artificial Sequence
  Description of Artificial Sequence Synthetic probe
taatacgactcactatagggtaatcttctatatcaacatcttacctaatacctttccttcatac

SEQ ID NO: 114 - DNA, 64 bp, Artificial Sequence
  Description of Artificial Sequence Synthetic probe
taatacgactcactatagggatcatacatacatacaaatctacatactactatcatcaacattc

SEQ ID NO: 115 - DNA, 64 bp, Artificial Sequence
  Description of Artificial Sequence Synthetic probe
taatacgactcactatagggctttcaattacaatactcattacattcttccttacaacccatcc

SEQ ID NO: 116 - DNA, 64 bp, Artificial Sequence
  Description of Artificial Sequence Synthetic probe
taatacgactcactatagggtcatttaccaatctttctttatacttacatttctattatttgac

SEQ ID NO: 117 - DNA, 64 bp, Artificial Sequence
  Description of Artificial Sequence Synthetic probe
taatacgactcactatagggtcatttcacaattcaattactcaaactacgaacggatccacagc

SEQ ID NO: 118 - DNA, 64 bp, Artificial Sequence
  Description of Artificial Sequence Synthetic probe
taatacgactcactatagggtacatcaacaattcattcaatacaattataacttcagtaacttc

SEQ ID NO: 119 - DNA, 64 bp, Artificial Sequence
  Description of Artificial Sequence Synthetic probe
taatacgactcactatagggcttctcattaacttacttcataatcctactaattacactaatcg

SEQ ID NO: 120 - DNA, 64 bp, Artificial Sequence
  Description of Artificial Sequence Synthetic probe
taatacgactcactatagggtacaaatcatcaatcactttaatcgagattggttgatgtatgag

SEQ ID NO: 121 - DNA, 64 bp, Artificial Sequence
  Description of Artificial Sequence Synthetic probe
taatacgactcactatagggctttaatctcaatcaatacaaatcaaagggttactcttgtattc

SEQ ID NO: 122 - DNA, 64 bp, Artificial Sequence
  Description of Artificial Sequence Synthetic probe
taatacgactcactatagggctttatcaatacatactacaatcacttgactttcaaccgcaccc

SEQ ID NO: 123 - DNA, 64 bp, Artificial Sequence
  Description of Artificial Sequence Synthetic probe
taatacgactcactatagggttcacttttcaatcaactttaatcgctgaagagcttcctgagtc

SEQ ID NO: 124 - DNA, 64 bp, Artificial Sequence
  Description of Artificial Sequence Synthetic probe
taatacgactcactatagggtacactttatcaaatcttacaatcccaaaggcagcttcaggcac

SEQ ID NO: 125 - DNA, 64 bp, Artificial Sequence
  Description of Artificial Sequence Synthetic probe
taatacgactcactatagggtcaattacttcactttaatcctttccaataaagcaatccttaac

SEQ ID NO: 126 - DNA, 64 bp, Artificial Sequence
  Description of Artificial Sequence Synthetic probe
taatacgactcactatagggattattcacttcaaactaatctactttcccggccaggccattgg

SEQ ID NO: 127 - DNA, 64 bp, Artificial Sequence
  Description of Artificial Sequence Synthetic probe
taatacgactcactatagggtcattcatatacataccaattcattaaggatgagtttcaagttg

SEQ ID NO: 128 - DNA, 64 bp, Artificial Sequence
  Description of Artificial Sequence Synthetic probe
taatacgactcactatagggtacattaccaataatcttcaaatctgatattctgaagcactttc

SEQ ID NO: 129 - DNA, 64 bp, Artificial Sequence
  Description of Artificial Sequence Synthetic probe
taatacgactcactatagggcaatttcatcattcattcatttcactgtccaagaacagtgtgtc

SEQ ID NO: 130 - DNA, 64 bp, Artificial Sequence
  Description of Artificial Sequence Synthetic probe
taatacgactcactatagggcaattcatttcattcacaatcaattttagacaagttcaatttag

SEQ ID NO: 131 - DNA, 64 bp, Artificial Sequence
  Description of Artificial Sequence Synthetic probe
taatacgactcactatagggcttttcatcttttcatctttcaatctggatggttttcgaaagtg

SEQ ID NO: 132 - DNA, 64 bp, Artificial Sequence
  Description of Artificial Sequence Synthetic probe
taatacgactcactatagggtcaacaatcttttacaatcaaatctcccagactacaaccggatc

SEQ ID NO: 133 - DNA, 64 bp, Artificial Sequence
  Description of Artificial Sequence Synthetic probe
taatacgactcactatagggaaacaaacttcacatctcaataattaagtggttacaggaagtcc

SEQ ID NO: 134 - DNA, 64 bp, Artificial Sequence
  Description of Artificial Sequence Synthetic probe
taatacgactcactatagggcaatataccaatatcatcatttacgggtgcgtctttgtatgttg

SEQ ID NO: 135 - DNA, 64 bp, Artificial Sequence
  Description of Artificial Sequence Synthetic probe
taatacgactcactatagggatacttcattcattcatcaattcagtccagccacccaggtgctc

SEQ ID NO: 136 - DNA, 64 bp, Artificial Sequence
  Description of Artificial Sequence Synthetic probe
taatacgactcactatagggtcaatcaattacttactcaaatacataacaatgcatcgtaaaac

SEQ ID NO: 137 - DNA, 64 bp, Artificial Sequence
  Description of Artificial Sequence Synthetic probe
taatacgactcactatagggtcaatcatctttatacttcacaatccgcctctctacccccagtc

SEQ ID NO: 138 - DNA, 64 bp, Artificial Sequence
  Description of Artificial Sequence Synthetic probe
taatacgactcactatagggtcatttcaatcaatcatcaacaattgaccaaccctccaactctg

SEQ ID NO: 139 - DNA, 64 bp, Artificial Sequence
  Description of Artificial Sequence Synthetic probe
taatacgactcactatagggtcaaaatctcaaatactcaaatcaacggatcatcttgtgaaaac

SEQ ID NO: 140 - DNA, 64 bp, Artificial Sequence
  Description of Artificial Sequence Synthetic probe
taatacgactcactatagggtcatcaatctttcaatttacttactggtaagcagctggaagatg

SEQ ID NO: 141 - DNA, 64 bp, Artificial Sequence
  Description of Artificial Sequence Synthetic probe
taatacgactcactatagggcttttcatcaataatcttacctttactccatgcagtcttgagtg

SEQ ID NO: 142 - DNA, 64 bp, Artificial Sequence
  Description of Artificial Sequence Synthetic probe
taatacgactcactatagggctacatattcaaattactacttacttctctgaaacgcaggattg

SEQ ID NO: 143 - DNA, 40 bp, Artificial Sequence
  Description of Artificial Sequence Synthetic probe
ctcctagtaagcctatatcttccctttagtgagggttaat

SEQ ID NO: 144 - DNA, 40 bp, Artificial Sequence
  Description of Artificial Sequence Synthetic probe
cataaaagtaaaaacccctttccctttagtgagggttaat

SEQ ID NO: 145 - DNA, 40 bp, Artificial Sequence
  Description of Artificial Sequence Synthetic probe
ccagatgcttacaccacatgtccctttagtgagggttaat

SEQ ID NO: 146 - DNA, 40 bp, Artificial Sequence
  Description of Artificial Sequence Synthetic probe
gtgaactacgactgctagaatccctttagtgagggttaat

SEQ ID NO: 147 - DNA, 40 bp, Artificial Sequence
  Description of Artificial Sequence Synthetic probe
ctccaagcttcagaatactttccctttagtgagggttaat

SEQ ID NO: 148 - DNA, 40 bp, Artificial Sequence
  Description of Artificial Sequence Synthetic probe
ctcaaagcaacgaagcctaatccctttagtgagggttaat

SEQ ID NO: 149 - DNA, 40 bp, Artificial Sequence
  Description of Artificial Sequence Synthetic probe
ctatggatccgagcatcttatccctttagtgagggttaat

SEQ ID NO: 150 - DNA, 40 bp, Artificial Sequence
  Description of Artificial Sequence Synthetic probe
ctcactctactcaacctcattccctttagtgagggttaat

SEQ ID NO: 151 - DNA, 40 bp, Artificial Sequence
  Description of Artificial Sequence Synthetic probe
ctagaaattgctcttctacttccctttagtgagggttaat

SEQ ID NO: 152 - DNA, 40 bp, Artificial Sequence
  Description of Artificial Sequence Synthetic probe
cgtactataatcatggcccgtccctttagtgagggttaat

SEQ ID NO: 153 - DNA, 40 bp, Artificial Sequence
  Description of Artificial Sequence Synthetic probe
cctaaactccaactccataatccctttagtgagggttaat

SEQ ID NO: 154 - DNA, 40 bp, Artificial Sequence
  Description of Artificial Sequence Synthetic probe
ccacttctataacagctatgtccctttagtgagggttaat

SEQ ID NO: 155 - DNA, 40 bp, Artificial Sequence
  Description of Artificial Sequence Synthetic probe
gttgatgatgttggagttattccctttagtgagggttaat

SEQ ID NO: 156 - DNA, 40 bp, Artificial Sequence
  Description of Artificial Sequence Synthetic probe
ctgatgtacagaaatcacattccctttagtgagggttaat

SEQ ID NO: 157 - DNA, 40 bp, Artificial Sequence
  Description of Artificial Sequence Synthetic probe
gccaggctgtcatcacaaagtccctttagtgagggttaat

SEQ ID NO: 158 - DNA, 40 bp, Artificial Sequence
  Description of Artificial Sequence Synthetic probe
caaaccaaatactgaaactgtccctttagtgagggttaat

SEQ ID NO: 159 - DNA, 40 bp, Artificial Sequence
  Description of Artificial Sequence Synthetic probe
caaggaagaccctaatctagtccctttagtgagggttaat

SEQ ID NO: 160 - DNA, 40 bp, Artificial Sequence
  Description of Artificial Sequence Synthetic probe
cattttgtgtctcccttttctccctttagtgagggttaat

SEQ ID NO: 161 - DNA, 40 bp, Artificial Sequence
  Description of Artificial Sequence Synthetic probe
catggctcctcccatctacatccctttagtgagggttaat

SEQ ID NO: 162 - DNA, 40 bp, Artificial Sequence
  Description of Artificial Sequence Synthetic probe
ccgttcaccgaccgccagtgtccctttagtgagggttaat

SEQ ID NO: 163 - DNA, 40 bp, Artificial Sequence
  Description of Artificial Sequence Synthetic probe
ctaaacatgcagcctatagatccctttagtgagggttaat

SEQ ID NO: 164 - DNA, 40 bp, Artificial Sequence
  Description of Artificial Sequence Synthetic probe
cctctagtgggaagaaatgatccctttagtgagggttaat

SEQ ID NO: 165 - DNA, 40 bp, Artificial Sequence
  Description of Artificial Sequence Synthetic probe
ggagttctccttgtttgtggtccctttagtgagggttaat

SEQ ID NO: 166 - DNA, 40 bp, Artificial Sequence
  Description of Artificial Sequence Synthetic probe
gtattataatgctgcaggattccctttagtgagggttaat

SEQ ID NO: 167 - DNA, 40 bp, Artificial Sequence
  Description of Artificial Sequence Synthetic probe
cgcagtggcatgttctggcttccctttagtgagggttaat

SEQ ID NO: 168 - DNA, 40 bp, Artificial Sequence
  Description of Artificial Sequence Synthetic probe
ctcaccctcccaaaagccactccctttagtgagggttaat

SEQ ID NO: 169 - DNA, 40 bp, Artificial Sequence
  Description of Artificial Sequence Synthetic probe
gggttaggtctttgaggttctccctttagtgagggttaat

SEQ ID NO: 170 - DNA, 40 bp, Artificial Sequence
  Description of Artificial Sequence Synthetic probe
ctttcccagcttgaattcaatccctttagtgagggttaat

SEQ ID NO: 171 - DNA, 40 bp, Artificial Sequence
  Description of Artificial Sequence Synthetic probe
cttcagaaggaaagaatgtttccctttagtgagggttaat

SEQ ID NO: 172 - DNA, 40 bp, Artificial Sequence
  Description of Artificial Sequence Synthetic probe
ctgcgcgtggctgccccttttccctttagtgagggttaat

SEQ ID NO: 173 - DNA, 40 bp, Artificial Sequence
  Description of Artificial Sequence Synthetic probe
cttctcaccatcagctaaagtccctttagtgagggttaat

SEQ ID NO: 174 - DNA, 40 bp, Artificial Sequence
  Description of Artificial Sequence Synthetic probe
ccacaccagagtctctgttctccctttagtgagggttaat

SEQ ID NO: 175 - DNA, 40 bp, Artificial Sequence
  Description of Artificial Sequence Synthetic probe
gccggactttgtctgactactccctttagtgagggttaat

SEQ ID NO: 176 - DNA, 40 bp, Artificial Sequence
  Description of Artificial Sequence Synthetic probe
ccctaagttgtcagcccaactccctttagtgagggttaat

SEQ ID NO: 177 - DNA, 40 bp, Artificial Sequence
  Description of Artificial Sequence Synthetic probe
cctccttaactgtactctcctccctttagtgagggttaat